(19) United States
(12) Patent Application Publication
(10) Pub. No.: US 2026/0317423 A1
(43) Pub. Date: Oct. 15, 2026

(54) COMPOSITIONS COMPRISING CARs AND METHODS OF USING THE SAME

(71) Applicant: Verismo Therapeutics, Inc., Philadelphia, PA (US)

(72) Inventors: Laura Alexandra JOHNSON, Philadelphia, PA (US); Nora YUCEL, Philadelphia, PA (US); Jun XU, Philadelphia, PA (US); Megan BLAIR, Philadelphia, PA (US)

(21) Appl. No.: 19/633,803

(22) Filed: Mar. 30, 2026

Related U.S. Application Data
(60) Provisional application No. 63/781,223, filed on Mar. 31, 2025.

(51) Int. Cl. C07K 16/28; A61K 35/17; C07K 14/705; C12N 15/11

(52) U.S. Cl. CPC C07K 16/2803; A61K 35/17; C07K 14/70503; C12N 15/111; C07K 2317/51; C07K 2317/565; C07K 2319/03

(57) ABSTRACT

The disclosure relates to compositions, nucleic acids, proteins, methods of making, and methods of treating concerning at least two KIR-CAR.

### CROSS-REFERENCE TO RELATED APPLICATION

The present application claims priority to U.S. Provisional Patent Application Ser. No. 63/781,223, filed on Mar. 31, 2025. The contents of the above-referenced applications are herein expressly incorporated by reference in their entirety, including any drawings.

### INCORPORATION OF THE SEQUENCE LISTING

This application contains a Sequence Listing, which is hereby incorporated herein by reference in its entirety. The accompanying Sequence Listing XML file, named “066340-504001WO.xml” was created on Mar. 30, 2026 and is 970,752 bytes.

### FIELD

This disclosure relates to nucleic acids encoding at least two CAR proteins, the CAR proteins, and cells and compositions comprising at least two CAR proteins disclosed herein. Additionally, methods of making the compositions and treating subjects using the compositions concerning at least two KIR-CAR are disclosed herein.

### BACKGROUND

Chimeric antigen receptor (CAR)-engineered T cells have demonstrated remarkable success in treating B-cell malignancies, with recent clinical data also highlighting their potential in addressing other diseases, including autoimmune disorders. However, many patients either relapse or fail to achieve a durable response with single tumor antigen-targeted CD19 CAR therapy due to various resistance and relapse mechanisms. See FIGS. 1A and 1B.

A primary mechanism for CAR therapy failure is antigen escape, which occurs through spontaneous antigen mutation, antigen splicing, antigen downregulation, or antigen epitope masking or CAR-mediated trogocytosis. Notably, a substantial number of patients with B-cell leukemia or lymphoma have experienced CD19-negative disease relapse following commercial CAR T-cell therapy. This underscores the urgent need for novel CAR T-cell approaches to overcome antigen escape and enhance therapeutic efficacy.

Both CD19 and CD20 are expressed on normal and malignant B cells, including in acute lymphoblastic leukemia, chronic lymphocytic leukemia, and Non-Hodgkin lymphomas. Targeting both tumor antigens simultaneously can enhance antigen recognition, mitigate the impact of antigen escape, and improve cytotoxicity against tumor cells with heterogeneous antigen expression levels.

Novel immunotherapies targeting two different antigens often require two separate vectors, each expressing a distinct CAR construct. This approach increases the complexity and cost of CAR T-cell therapy. Tandem CARs using a single-chain design are currently being tested; however, differences in scFv binding affinities from two different antigen-binding domains can lead to suboptimal signaling, and the spatial proximity of antigen-binding domains can interfere with effective antigen binding.

### SUMMARY

Aspects of the disclosure relate to nucleic acids, proteins, cells, and compositions comprising the foregoing that employ KIR amino acid domains within a CAR to target malignant cells expressing CD19 or CD20 or both, and to induce cytotoxic responses in autologous T cells comprising the KIR amino acid domains. Additional aspects of the disclosure relate to a single nucleic acid molecule comprising a nucleic acid sequence encoding a CAR that targets cells expressing CD20 and a CAR targeting cells encoding CD19, such that expression of the nucleic acid molecule in a cell, results in the cell expressing two CAR amino acid sequences. Additional aspects of the disclosure provide methods of making cells comprising one or more CAR amino acid sequences, and methods of treatment of hyperproliferative disorders or autoimmune diseases by administering compositions comprising cells expressing the KIR amino acid domains. Without being limited to any particular theory, it was unexpectedly discovered that certain aspects of embodiments disclosed herein can provide enhanced efficacy in reducing and/or eliminating certain cancers, while also reducing the likelihood of T cell exhaustion and tonicity. Aspects of the disclosure relate to reducing disease relapse rates, and/or providing improved treatments for alleviating, reducing, mitigating, and/or treating certain hyperproliferative disorders. Additionally, it was discovered that certain embodiments disclosed herein include cells having a reduced T cell exhaustion rate while also providing one or more of the foregoing benefits.

In accordance with some aspects of the disclosure, provided are compositions comprising a nucleic acid encoding a protein and/or compositions comprising a protein and or compositions comprising a protein, wherein the protein comprises a first and second KIR domain. In accordance with some aspects, the composition comprises a first nucleic acid sequence encoding a first fusion protein. In some embodiments, the first fusion protein may include an antigen-binding domain comprising an anti-CD19 amino acid sequence, a transmembrane domain comprising a KIR polypeptide, and a cytoplasmic domain comprising a KIR polypeptide. The composition may also comprise a second nucleic acid sequence encoding a second fusion protein, the second fusion protein may include an antigen-binding domain comprising an anti-CD20 amino acid sequence, a transmembrane domain comprising a KIR polypeptide, and a cytoplasmic domain comprising a KIR polypeptide. In some embodiments, the first fusion protein includes an antigen-binding domain comprising a CD19-targeting amino acid sequence, a transmembrane domain comprising a KIR polypeptide, and a cytoplasmic domain comprising a KIR polypeptide; and the second fusion protein may include an antigen-binding domain comprising a CD20 targeting-domain, a transmembrane domain comprising a KIR polypeptide, and a cytoplasmic domain comprising a KIR polypeptide. In some embodiments, the disclosure relates to a single nucleic acid molecule comprising a first and second expressible nucleic acid sequence, the first nucleic acid sequence encoding a first fusion protein comprising an anti-CD19 amino acid sequence and the second nucleic acid sequence encoding a second fusion protein comprising an anti-CD20 amino acid sequence, wherein the first and second nucleic acid sequences are positioned within the nucleic acid molecule on the same open reading frame and are bicistronic. In some embodiments, the single nucleic acid molecule further comprises a third nucleic acid sequence encoding DAP12, wherein the first and second nucleic acid sequences and the third nucleic acid sequence are positioned within the nucleic acid molecule on the same open reading frame and are tricistronic.

In some embodiments, the compositions disclosed herein further comprise a third nucleic acid sequence encoding a DAP12 polypeptide. In some embodiments, the first nucleic acid sequence and/or the second nucleic acid sequence are free of a nucleic acid sequence encoding one or more of a 41BB polypeptide, an anti-CD22 binding sequence, and a CD28 domain. In some embodiments, the first and second nucleic acid sequences each further comprise a nucleic acid sequence encoding a KIR2DS2 domain. The antigen-binding domain of the first nucleic acid sequence and the second nucleic acid sequence may comprise at least a portion of a KIR polypeptide independently chosen from KIR2DS2, KIR2DS1, KIR2DS3, KIR2DS4, KIR2DS5, KIR3DS1, a combination thereof, and/or functional variants thereof. In some embodiments, the transmembrane domain of the first nucleic acid sequence and the second nucleic acid sequence comprises at least a portion of a KIR polypeptide independently chosen from KIR2DS2, KIR2DS1, KIR2DS3, KIR2DS4, KIR2DS5, KIR3DS1, and/or a combination thereof. A KIR polypeptide may comprise SEQ ID NO: 668, SEQ ID NO: 669, SEQ ID NO: 670, SEQ ID NO: 671, SEQ ID NO: 672, SEQ ID NO: 673, or functional variants thereof comprising at least about 75%, 80%, 85%, 90%, 91%, 92%, 93%, 94%, 95%, 96%, 97%, 98% or about 99% sequence identity to SEQ ID NO: 668, SEQ ID NO: 669, SEQ ID NO: 670, SEQ ID NO: 671, SEQ ID NO: 672, SEQ ID NO: 673.

The disclosure also relates to a composition comprising a cell, wherein the cell comprises a CD19-targeting domain comprising an amino acid sequence chosen from SEQ ID NOS: 87 through SEQ ID NO: 247 or functional variants thereof that comprise at least about 75% sequence identity to SEQ ID NOS: 87 through 247, respectively. In some embodiments, the CD19-targeting domain comprises the amino acid sequence of one of GNS, AND, ANI, SDG, GSS, GNT, GYN, ADS, GSH, KDT, SSS, LVS, QVS, RDT, SVS, NND, SNK, and EAS.

The disclosure further relates to a composition comprising a cell, wherein the cell comprises a first and second fusion protein; wherein the first fusion protein comprises a CD20-targeting domain comprising an amino acid sequence chosen from one of SEQ ID NOS: 504 through 526 or a functional variant thereof, that comprises at least about 75% sequence identity to the respective amino acid sequence of SEQ ID NOS: 504 through 526.

The disclosure relates to a composition comprising a cell, wherein the cell comprises any nucleic acid molecule disclosed herein. In some embodiments, the cell comprises a nucleic acid molecule comprising a nucleic acid sequence encoding a CD19-targeting domain, wherein the CD19-targeting domain is chosen from SEQ ID NOS: 87 through SEQ ID NO: 247 or functional variants thereof that comprise at least about 75% sequence identity to SEQ ID NOS: 87 through 247, respectively.

The disclosure further relates to a composition comprising a cell comprising a nucleic acid molecule comprising a nucleic acid sequence encoding a CD20-targeting domain, wherein the CD20-targeting domain is chosen from one of SEQ ID NOS: 504 through 526 or a functional variant thereof, wherein the functional variant thereof comprises at least about 75% sequence identity to the respective amino acid sequence of SEQ ID NOS: 504 through 526.

The disclosure also relates to a composition comprising a cell, wherein the cell comprises a nucleic acid molecule comprising a nucleic acid sequence encoding a CD19-targeting domain and a nucleic acid sequence encoding a CD20-targeting domain. In some embodiments, the nucleic acid molecule further comprises a nucleic acid sequence encoding a transmembrane domain and a cytoplasmic domain, wherein each of the transmembrane domain and the cytoplasmic domain comprise a fusion protein that is a KIR2DS2 domain. The transmembrane and the cytoplasmic domain may include one or more DAP12 association sequences. Each of the first fusion protein and the second fusion protein may comprise a DAP12 association sequence. In some embodiments, the DAP12 association sequence of one or both of the first fusion protein and the second fusion protein comprises an amino acid sequence of SEQ ID NO: 753 or a functional variant thereof that comprises at least about 75% sequence identity to one of SEQ ID NO: 753.

Compositions of the disclosure may also include a nucleic acid molecule comprising a nucleic acid sequence encoding a CD19-targeting domain, a CD20 targeting domain, and a transmembrane domain, wherein the transmembrane domain is configured to associate with DAP12 when the CD19-targeting domain is associated with CD19. Additionally or alternatively, the transmembrane domain is configured to associate with DAP12 when the CD20 targeting domain is associated with CD20. Additionally or alternatively, the transmembrane domain is configured to associate with DAP12 when the CD19 and the CD20 targeting domain is associated with CD19 and CD20.

The composition may, in certain embodiments, further comprise a first nucleic acid molecule comprising a first expressible nucleic acid sequence and a second expressible nucleic acid sequence, where the first nucleic acid sequence and the second nucleic acid sequence are non-contiguous. For instance, the first nucleic acid and the second nucleic acid may be linked by a nucleic acid encoding a cleavable linker sequence. In at least one embodiment, the cleavable linker sequence is a P2A linker or a furin sequence. In some embodiments, the first nucleic acid molecule comprises, in the 5′ to 3′ orientation, a regulatory sequence for the first nucleic acid sequence, a P2A linker encoding sequence, and the second nucleic acid sequence. In further embodiments, the first nucleic acid molecule comprises, in the 5′ to 3′ orientation, a regulatory sequence for the first nucleic acid sequence, a furin linker encoding sequence, and the second nucleic acid sequence.

Compositions of the disclosure also relate to a nucleic acid molecule comprising a nucleic acid sequence encoding a scFv domain comprising a CD19-targeting domain comprising an amino acid sequence of one of SEQ ID NOS: 271 through 356, or a functional variant thereof that comprises at least about 75% sequence identity to the SEQ ID NOS: 271 through 356. The anti-CD20 amino acid sequence may, additionally or alternatively, comprise an scFv domain comprising the amino acid sequence of one of SEQ ID NOS: 459 through 503 or a functional variant thereof that comprises at least about 75% sequence identity to the respective one of SEQ ID NOS: 459 through 503.

Additional aspects of the disclosure relate to fusion proteins. For instance, the composition comprises an antigen-binding domain comprising a CD19 targeting domain, a transmembrane domain comprising a KIR polypeptide, and a cytoplasmic domain comprising a KIR polypeptide, wherein the KIR polypeptide associates with DAP12. In some embodiments, the anti-CD19 amino acid sequence comprises the amino acid sequence of one of SEQ ID NOS: 87-247 or a functional variant thereof that comprises at least about 75% sequence identity to the respective amino acid sequence of SEQ ID NOS: 87-247. In certain embodiments, the anti-CD19 amino acid sequence comprises the amino acid sequence of one of GNS, AND, ANI, SDG, GSS, GNT, GYN, ADS, GSH, KDT, SSS, LVS, QVS, RDT, SVS, NND, SNK, and/or EAS.

The fusion protein may be adapted such that, in some embodiments, each of the transmembrane domain and cytoplasmic domain comprise a KIR2DS2 domain. In some embodiments, the DAP12 association sequence of a KIR2DS2 polypeptide comprises SEQ ID NO: 753 or a functional variant thereof that comprises at least about 75% sequence identity to SEQ ID NO: 753.

In some embodiments, the transmembrane domain of the first nucleic acid sequence is configured to associate with DAP12 when the antigen-binding domain is associated with CD19. Additionally or alternatively, in some cases, at least a portion of the cytoplasmic domain is in an active state when CD19 is bound to the antigen-binding domain.

The fusion protein may further comprise a second antigen binding domain comprising an anti-CD20 amino acid sequence comprising the amino acid sequence of one of SEQ ID NOS: 459 through 503 or a functional variant thereof that comprises at least about 75% sequence identity to the respective amino acid sequence of SEQ ID NOS: 459 through 503. The disclosure also relates to a fusion protein comprising a first antigen binding domain and a second antigen binding domain that are non-contiguous and, e.g., linked by a cleavable linker sequence. Examples of cleavable linker sequences include a P2A linker or a furin sequence.

In some embodiments, the fusion protein comprises, in an amino to carboxy orientation, an IgK, CD8a, or GMCSFR leader sequence, a first antigen binding domain, a first transmembrane domain, a first cytoplasmic domain, a P2A linker, and a second antigen binding domain, a second transmembrane domain and a second cytoplasmic domain, wherein the first antigen binding domain comprises a CD19-targeting domain and the second antigen binding domain comprises a CD20-targeting domain. In some embodiments, the fusion protein comprises, in an amino to carboxy orientation: an IgK leader sequence, a first antigen binding domain, a transmembrane domain, a first cytoplasmic domain, a P2A, T2A, F2A or E2A linker, and a second antigen binding domain, a second transmembrane domain, and a second cytoplasmic domain, wherein the first antigen binding domain comprises a CD19-targeting domain and the second antigen binding domain comprises a CD20-targeting domain.

The anti-CD19 amino acid sequence of the fusion protein may comprise an scFv domain comprising an amino acid sequence of one of SEQ ID NOS: 271-356 or a functional variant thereof that comprises at least about 75% sequence identity to the respective one of SEQ ID NOS: 271-356.

In some embodiments, the fusion protein comprises, in an amino to carboxy orientation: an IgK leader sequence, an antigen-binding domain comprising anti-CD19 amino acid sequence, a transmembrane domain, and a cytoplasmic domain, a furin linker, a second antigen binding domain comprising an anti-CD20 amino acid sequence, a second transmembrane domain, and a second cytoplasmic domain. The transmembrane domain may comprise a KIR polypeptide while the cytoplasmic domain comprises a KIR polypeptide in certain embodiments of the fusion protein. The second transmembrane domain may comprise a KIR polypeptide while the second cytoplasmic domain comprises a KIR polypeptide in certain embodiments of the fusion protein. The first and second transmembrane domains may separately comprise a KIR polypeptide while the first and second cytoplasmic domains separately comprise a KIR polypeptide in certain embodiments of the fusion protein.

The fusion protein may further comprise a first extracellular linker domain between the first antigen binding domain and the transmembrane domain. In some instances, the fusion protein further includes a second extracellular linker domain between the second antigen binding domain and the second transmembrane domain. In some embodiments, the fusion protein is no more than about 22,000 kD in weight.

Additional aspects of the disclosure relate to a cell. For example, aspects of the disclosure relate to a cell comprising a single nucleic acid molecule comprising a first nucleic acid sequence and a second nucleic acid molecule comprising a second nucleic acid sequence, wherein the first nucleic acid sequence encodes a first CAR and the second nucleic acid sequence encodes a second CAR, such as those described herein. Alternatively, the cell may comprise a single, multicistronic nucleic acid molecule comprising the first and the second nucleic acid sequence, wherein the first nucleic acid sequence encodes a first CAR and the second nucleic acid sequence encodes a second CAR, each CAR comprising an antigen-binding domain, transmembrane domain and a cytoplasmic domain; wherein the first CAR comprises a CD19 targeting domain and the second CAR comprises a CD20 targeting domain.

The cell may, additionally or alternatively, include a fusion protein having an antigen-binding domain comprising an anti-CD19 amino acid sequence, a transmembrane domain comprising a KIR polypeptide, and a cytoplasmic domain comprising a KIR polypeptide targeting DAP12.

The fusion protein of the cell may comprise, in an amino to carboxy orientation: an IgK leader sequence, an antigen-binding domain comprising anti-CD19 amino acid sequence, a transmembrane domain, and a cytoplasmic domain, a P2A linker, a second antigen binding domain comprising an anti-CD20 amino acid sequence, a second transmembrane domain, and a second cytoplasmic domain. The transmembrane domain may comprise a KIR polypeptide while the cytoplasmic domain comprises a KIR polypeptide in certain embodiments of the fusion protein. The second transmembrane domain may comprise a KIR polypeptide while the second cytoplasmic domain comprises a KIR polypeptide in certain embodiments of the fusion protein. The first and second transmembrane domains may separately comprise a KIR polypeptide while the first and second cytoplasmic domains separately comprise a KIR polypeptide in certain embodiments of the fusion protein.

In some embodiments, the fusion protein of the cell may comprise, in an amino to carboxy orientation: an IgK leader sequence, an antigen-binding domain comprising anti-CD19 amino acid sequence, a transmembrane domain, and a cytoplasmic domain, a furin linker, a second antigen binding domain comprising an anti-CD20 amino acid sequence, a second transmembrane domain, and a second cytoplasmic domain. The transmembrane domain may comprise a KIR polypeptide while the cytoplasmic domain comprises a KIR polypeptide targeting DAP12 in certain embodiments of the fusion protein. The second transmembrane domain may comprise a KIR polypeptide while the second cytoplasmic domain comprises a KIR polypeptide targeting DAP12 in certain embodiments of the fusion protein. The first and second transmembrane domains may separately comprise a KIR polypeptide while the first and second cytoplasmic domains separately comprise a KIR polypeptide targeting DAP12 in certain embodiments of the fusion protein.

In some embodiments, the cell is a T cell, B cell, NK cell, monocyte, dendritic cell, or macrophage. In some embodiments, the cell is a tumor infiltrating T cell. In some embodiments, the cell is an autologous T cell modified in vitro. In some embodiments, the cell has a surface expression of the first and second antigen binding domains from about 1,000 to about 10,000 copies after about 20 days in vivo. In some embodiments, the cell has a surface expression of the first and second antigen binding domains are from about 1,000 to about 100,000 copies after about 20 days in vivo. In some embodiments, the cell has a surface expression of the first and second antigen binding domains are from about 100 to about 1,000,000 copies after about 20 days in vivo.

In some embodiments, the disclosure relates to a cell comprising a first and a second exogenous protein. The first exogenous protein may comprise a first antigen-binding domain comprising an anti-CD19 amino acid sequence, a first transmembrane domain comprising a KIR polypeptide configured to associate with DAP12, and a cytoplasmic domain comprising a KIR polypeptide configured to associate with DAP12. The second exogenous protein may comprise a second antigen-binding domain comprising an anti-CD20 amino acid sequence, a second transmembrane domain comprising a KIR polypeptide configured to associate with DAP12, and a second cytoplasmic domain comprising a KIR polypeptide configured to associate with DAP12. Additionally, in some embodiments, the cell further comprises a DAP12 polypeptide. The anti-CD19 amino acid sequence may comprise an amino acid sequence of one of SEQ ID NOS: 87-247 or a functional variant thereof, which has a sequence identify of about 75% or more to the respective amino acid sequence of SEQ ID NOS: 87-247. In some instances, the anti-CD19 amino acid sequence comprises the amino acid sequence of one of GNS, AND, ANI, SDG, GSS, GNT, GYN, ADS, GSH, KDT, SSS, LVS, QVS, RDT, SVS, NND, SNK, and/or EAS. In some embodiments, the anti-CD19 amino acid sequence comprises an scFv domain comprising the amino acid sequence of one of SEQ ID NOS: 271-356 or a functional variant thereof, which has a sequence identify of about 75% or more to the respective one of SEQ ID NOS: 271-356.

The anti-CD20 amino acid sequence of the second exogenous protein may comprise an amino acid sequence of one of SEQ ID NOS: 504-526 or a functional variant thereof, which has a sequence identify of about 75% or more to the respective amino acid sequence of SEQ ID NOS: 504-526. In some embodiments, the anti-CD20 amino acid sequence comprises the amino acid sequence of one of 504-526. In some embodiments, the anti-CD20 amino acid sequence is an scFv domain comprising the amino acid sequence of one of SEQ ID NOS: 459-503 or a functional variant thereof that comprises at least about 75% sequence identity to the respective one of SEQ ID NOS: 459-503. In some embodiments, the first and second transmembrane domains are identical. In some embodiments, the first and second cytoplasmic domains are identical. In some embodiments, the first and second transmembrane domains associate with DAP12 in the presence of CD19 and/or CD20. The cell comprising the first and the second exogenous protein may be a T cell, B cell, NK cell, monocyte, dendritic cell, or macrophage such as, e.g., a tumor infiltrating T cell. In some embodiments, the cell is an autologous T cell modified in vitro. The first and/or second exogenous protein may further comprise a co-stimulatory domain. In some embodiments, the co-stimulatory domain comprises a polypeptide comprising a domain from 4-1BB, OX40, CD2, CD27, or CD28. In some embodiments, the surface expression of the first and second antigen binding domains are from about 1,000 to about 10,000 copies after about 20 days in vivo. In some embodiments, the surface expression of the first and second antigen binding domains are from about 1,000 to about 100,000 copies after about 20 days in vivo. In some embodiments, the surface expression of the first and second antigen binding domains are from about 100 to about 1,000,000 copies after about 20 days in vivo.

The disclosure relates to a pharmaceutical composition. In some embodiments, the pharmaceutical composition comprises a therapeutically effective amount of one or more cells disclosed herein and a pharmaceutically acceptable carrier. In some embodiments, the therapeutically effective amount is from about 1×10⁶ to about 100×10⁶ cells. In some embodiments, the therapeutically effective amount is from about 1×10⁶ to about 3×10⁸ cells. In some embodiments, the therapeutically effective amount is from about 1×10⁶ to about 3×10¹⁰ cells. In some embodiments, the therapeutically effective amount is from about 10×10⁶ to about 50×10⁶ cells. In some embodiments, the pharmaceutically acceptable carrier comprises phosphate-buffered saline at a pH of about from about 6.5 to about 8.0.

Further aspects of the disclosure relate to methods of treatment of a B cell-mediated disorder in a subject in need thereof. In some embodiments, the B cell-mediated disorder is selected from one described in Alaggio, R. et al. “The 5th edition of the World Health Organization Classification of Haematolymphoid Tumors: Lymphoid Neoplasms” (2022) Leukemia 36(7): 1720-1748, which is incorporated herein by reference in its entirety. In some embodiments, the method of treatment is for a B cell lymphoma, B-cell leukemia, acute lymphoblastic leukemia, and chronic lymphocytic leukemia, small lymphoblastic leukemia, B-cell lymphoma or Burkitt lymphoma. Additional aspects of the disclosure relate to methods of treating B cell lymphoma in a subject need thereof, the method comprising administering to the subject a pharmaceutical composition comprising a therapeutically effective amount of cells disclosed herein comprising a nucleic acid molecule comprising a nucleic acid sequence encoding a first CAR and a second CAR.

In some embodiments, the method of treatment is for an autoimmune disorder or disease, the method comprising administering to the subject a pharmaceutical composition comprising a therapeutically effective amount of cells disclosed herein comprising a nucleic acid molecule comprising a nucleic acid sequence encoding a first CAR and a second CAR. In some embodiments, the autoimmune disorder is antibody mediated. In some embodiments, administration of the modified immune cell depletes autoimmune B cells. In some embodiments, the autoimmune disorder is rheumatoid arthritis, systemic lupus erythematosus (SLE), lupus nephritis (LN), idiopathic/autoimmune thrombocytopenia purpura (ITP), idiopathic thrombotic thrombocytopenia purpura (TTP), pemphigus-related disorders, diabetes, scleroderma, myasthenia gravis, multiple sclerosis, vasculitis, or autoimmune hemolytic anemia.

In some embodiments, the method of treatment is for an alloantibody-mediated disorder or disease, the method comprising administering to the subject a pharmaceutical composition comprising a therapeutically effective amount of cells disclosed herein comprising a nucleic acid molecule comprising a nucleic acid sequence encoding a first CAR and a second CAR. In some embodiments, the administration of the modified immune cell depletes B cells that produce alloantibodies. In some embodiments, the alloantibody-mediated disorder is selected from the group consisting of solid organ transplant immune incompatibility, resistance to enzyme replacement therapy or acute or delayed hemolytic reactions to transfusion, and chronic alloantibody mediated rejection (CAMR), organ transplant immune incompatibility hemophilia (hemophilia A or B), lysosomal storage diseases (LSD), urea cycle disorder, adenosine deaminase deficiency, neuronal ceroid lipofuscinoses (NCL), hyperammonemia, and chronic graft-versus-host disease (cGvHD). In certain embodiments, the lysosomal storage disease is selected from the group consisting of glycogen storage disease, Gaucher's disease, Niemann-Pick disease, Fabry's disease, and mucopolysaccharidosis (MPS) I, MPS II, and MPS VI.

The methods of treatment typically comprise administering to the subject a pharmaceutical composition, such as one of the pharmaceutical compositions disclosed herein and/or a pharmaceutical composition having one or one or more nucleic acids, one or more proteins, and/or one or more cells disclosed herein. The method may further include the step of administering a chemotherapeutic agent simultaneously with, before, or after administration of the pharmaceutical composition.

The method of treatment may further comprise a step of allowing a sufficient period of time for the therapeutically effective amount of cells to bind and to kill a target cancer cell. In some embodiments, the method of treatment is free of off-target binding from about 3% to about 10% of the total association between the therapeutically effective amount of cells and a subject's cells. In some embodiments, the method is free of CD28 stimulation. In some embodiments, the method comprises CD28 stimulation.

The method of treatment may include administering the pharmaceutical composition by intrapleural administration, intravenous administration, intraarterial administration, locoregional administration at the site of a surgical resection or combinations thereof. In some embodiments, the therapeutically effective amount of cells is from about 1×10⁶ to about 100×10⁶ cells. In some embodiments, the therapeutically effective amount is from about 1×10⁶ to about 3×10⁸ cells. In some embodiments, the therapeutically effective amount is from about 1×10⁶ to about 3×10¹⁰ cells. In some embodiments, the therapeutically effective amount is from about 10×10⁶ to about 50×10⁶ cells.

Further aspects of the disclosure relate to methods of inducing antigen-specific T cell activation against a cell expressing CD19 and CD20. In some embodiments, the method of inducing comprises exposing a cell disclosed herein to a cell expressing CD19 and CD20 or an epitope of CD19 and CD20. The step of exposing may be performed in vivo. In some embodiments, the method further comprises transducing a cell with a composition herein before the step of exposing. The method may also comprise extracting a sample of lymphocytes from a subject and isolating T cells from the sample prior to the step of exposing.

The disclosure further provides methods of reducing a relapse rate of cancer in a subject in need thereof, wherein, in some embodiments, the subject is a mammal, such as a human. In some embodiments, the method comprises administering to the subject a pharmaceutical composition, such as one of the pharmaceutical compositions comprising a cell disclosed herein and/or a pharmaceutical composition having one or one or more nucleic acid molecules and/or one or more proteins disclosed herein. The method of reducing a relapse rate of cancer may include administering the pharmaceutical composition by intrapleural administration, intravenous administration, intraarterial administration, locoregional administration at the site of a surgical resection or combinations thereof. The pharmaceutical composition may be administered in a therapeutically effective amount of cells from about 1×10⁶ to about 100×10⁶ cells. The pharmaceutical composition may be administered in a therapeutically effective amount of cells from about 1×10⁶ to about 3×10⁸ cells. The pharmaceutical composition may be administered in a therapeutically effective amount of cells from about 1×10⁶ to about 3×10¹⁰ cells. In some embodiments, the therapeutically effective amount is from about 10×10⁶ to about 50×10⁶ cells. In some embodiments, the method further comprises a step of administering a chemotherapeutic agent simultaneously with, before or after administration of the pharmaceutical composition. In some embodiments, the methods of treatment are free of CD28 stimulation. In some embodiments, the cancer is B cell lymphoma.

In embodiments, the disclosure relates to a method of treatment for an autoimmune disease in a subject in need thereof. In some embodiments, the method comprises administering to the subject a pharmaceutical composition herein. In some embodiments, method is free of CD28 stimulation. In some embodiments, the method further comprises a step of administering a chemotherapeutic agent simultaneously with, before, or after administration of the pharmaceutical composition. In some embodiments, the autoimmune disease is rheumatoid arthritis, systemic lupus erythematosus (SLE), lupus nephritis (LN), idiopathic/autoimmune thrombocytopenia purpura (ITP), idiopathic thrombotic thrombocytopenia purpura (TTP), pemphigus-related disorders, diabetes, scleroderma, myasthenia gravis, multiple sclerosis, vasculitis, or autoimmune hemolytic anemia.

In some embodiments, the disclosure relates to a method of treatment for an alloantibody-mediated disorder or disease in a subject in need thereof. The method comprises administering to the subject a pharmaceutical composition herein. In some embodiments, the method is free of CD28 stimulation. In some embodiments, the method further comprises a step of administering a chemotherapeutic agent simultaneously with, before, or after the administration of the pharmaceutical composition. In some embodiments, the alloantibody-mediated disorder or disease is solid organ transplant immune incompatibility, resistance to enzyme replacement therapy, hemolytic reactions to transfusion, acute hemolytic reactions to transfusion, delayed hemolytic reactions to transfusion, chronic alloantibody mediated rejection (CAMR), organ transplant immune incompatibility hemophilia, organ transplant immune incompatibility hemophilia A, organ transplant immune incompatibility hemophilia B, lysosomal storage diseases (LSD), urea cycle disorder, adenosine deaminase deficiency, neuronal ceroid lipofuscinoses (NCL), hyperammonemia, chronic graft-versus-host disease (cGvHD), glycogen storage disease, Gaucher's disease, Niemann-Pick disease, Fabry's disease, and mucopolysaccharidosis (MPS) I, MPS II, and/or MPS VI.

Also provided herein is a composition comprising one or more recombinant nucleic acid molecules comprising a. a first nucleic acid sequence encoding a first fusion protein comprising: (i) an antigen-binding domain comprising an anti-CD19 amino acid sequence; (ii) a transmembrane domain comprising a KIR polypeptide; and (iii) a cytoplasmic domain comprising a KIR polypeptide; and b. a second nucleic acid sequence encoding a second fusion protein comprising: (iii) an antigen-binding domain comprising an anti-CD20 amino acid sequence; (iv) a transmembrane domain comprising a KIR polypeptide; and (v)
a cytoplasmic domain comprising a KIR polypeptide.

In some embodiments, the composition further comprises a third nucleic acid sequence encoding a DAP12 polypeptide.

In some embodiments, the first and second nucleic acid sequences are free of a nucleic acid sequence encoding a 41BB polypeptide, an anti-CD22 binding sequence, a CD28 domain.

In some embodiments, the first and second nucleic acid sequences each comprise a nucleic acid sequence encoding a KIR2DS2 domain.

In some embodiments, one or both of the transmembrane domains of the first and second nucleic acid sequences comprises at least a portion of a KIR polypeptide independently selected from the group consisting of KIR2DS2, KIR2DS1, KIR2DS3, KIR2DS4, KIR2DS5, and KIR3DS1.

In some embodiments, the transmembrane domain of the first and second nucleic acid sequences comprises at least a portion of a KIR polypeptide independently selected from the group consisting of KIR2DS2, KIR2DS1, KIR2DS3, KIR2DS4, KIR2DS5, and KIR3DS1.

In some embodiments, the portion of a KIR polypeptide comprises at least about 75% sequence identity to SEQ ID NO: 266 or 744.

In some embodiments, one or both of the transmembrane domains of the first and second nucleic acid sequences comprises at least a portion of a KIR polypeptide independently selected from KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3 and two pseudogenes, KIR2DP1 and KIR3DP1.

In some embodiments, the anti-CD19 amino acid sequence comprises the amino acid sequence of any one of SEQ ID NOS: 87-247 or a functional variant thereof comprising at least about 75% sequence identity to the respective amino acid sequence of any one of SEQ ID NOS: 87-247, or wherein the anti-CD19 amino acid sequence comprises the amino acid sequence of one of GNS, AND, ANI, SDG, GSS, GNT, GYN, ADS, GSH, KDT, SSS, LVS, QVS, RDT, SVS, NND, SNK, and EAS.

In some embodiments, the antigen-binding domain comprising an anti-CD19 amino acid sequence comprises an anti-CD19 antibody or an antigen-binding fragment thereof.

In some embodiments, the antigen-binding domain comprising an anti-CD19 amino acid sequence is an antigen-binding fragment of an anti-CD19 antibody.

In some embodiments, the antigen-binding domain comprising an anti-CD19 amino acid sequence is a single chain variable fragment (scFv).

In some embodiments, the antigen-binding domain comprising an anti-CD19 amino acid sequence comprises a heavy chain variable region (VH) that comprises a first heavy chain complementarity determining region (HCDR1) having the sequence GFTLSNYG (SEQ ID NO: 163), a second heavy chain complementarity determining region (HCDR2) having the sequence IRSGGNTY (SEQ ID NO: 164), and a third heavy chain complementarity determining region (HCDR3) having the sequence SQWGDWGHFEY (SEQ ID NO: 123).

In some embodiments, the antigen-binding domain comprising an anti-CD19 amino acid sequence comprises a light chain variable region (VL) that comprises a first light chain complementarity determining region (LCDR1) having the sequence RSNIGANY (SEQ ID NO: 124), a second light chain complementarity determining region (LCDR2) having the sequence GYN, and a third light chain complementarity determining region (LCDR3) having the sequence STWDSSLNAYV (SEQ ID NO: 165).

In some embodiments, the antigen-binding domain comprising an anti-CD19 amino acid sequence comprises a VH comprising a sequence having at least 90%, 95%, 96%, 97%, 98%, 99%, or 100% sequence identity to the sequence EVPLVESGGDLVKPGGSLRLSCVASGFTLSNYGMSWVRQSPGKGLQWVAAIRSGGNT YYADAVKGRFTISRDNAKNTLYLQMNSLRAEDTAVYYCASQWGDWGHFEYWGQGTL VTVSS (SEQ ID NO: 331) and a VL comprising a sequence having at least 90%, 95%, 96%, 97%, 98%, 99%, or 100% sequence identity to the sequence QSALTQTASVSGSLGQKVTISCTGSRSNIGANYVGWYQQVPGIGPRTVIFGYNYRPSGV PDRFSSSKSGNSATLTISGLQAEDEAEYYCSTWDSSLNAYVFGSGTQLTVL (SEQ ID NO: 288).

In some embodiments, the antigen-binding domain comprising an anti-CD19 amino acid sequence comprises a VH having the sequence EVPLVESGGDLVKPGGSLRLSCVASGFTLSNYGMSWVRQSPGKGLQWVAAIRSGGNT YYADAVKGRFTISRDNAKNTLYLQMNSLRAEDTAVYYCASQWGDWGHFEYWGQGTL VTVSS (SEQ ID NO: 331) and a VL having the sequence QSALTQTASVSGSLGQKVTISCTGSRSNIGANYVGWYQQVPGIGPRTVIFGYNYRPSGV PDRFSSSKSGNSATLTISGLQAEDEAEYYCSTWDSSLNAYVFGSGTQLTVL (SEQ ID NO: 288).

In some embodiments, the VH and VL are linked by a linker comprising the sequence GGGSSRSSSSGGGGSGGGG (SEQ ID NO: 413).

In some embodiments, the VH and VL are linked by a linker comprising the sequence GSTSGGGSGGGSGGGGSS (SEQ ID NO: 806).

In some embodiments, the antigen-binding domain comprising an anti-CD20 amino acid sequence comprises a sequence having at least 90%, 95%, 96%, 97%, 98%, 99%, or 100% sequence identity to the sequence QSALTQTASVSGSLGQKVTISCTGSRSNIGANYVGWYQQVPGIGPRTVIFGYNYRPSGV PDRFSSSKSGNSATLTISGLQAEDEAEYYCSTWDSSLNAYVFGSGTQLTVLGGGSSRSSS SGGGGSGGGGEVPLVESGGDLVKPGGSLRLSCVASGFTLSNYGMSWVRQSPGKGLQW VAAIRSGGNTYYADAVKGRFTISRDNAKNTLYLQMNSLRAEDTAVYYCASQWGDWG HFEYWGQGTLVTVSS (SEQ ID NO: 61).

In some embodiments, the anti-CD20 amino acid sequence comprises the amino acid sequence of one of SEQ ID NOS: 504-526 or a functional variant thereof that comprises at least about 75% sequence identity to the respective amino acid sequence of SEQ ID NOS: 504-526.

In some embodiments, the antigen-binding domain comprising an anti-CD20 amino acid sequence comprises an anti-CD20 antibody or an antigen-binding fragment thereof.

In some embodiments, the antigen-binding domain comprising an anti-CD20 amino acid sequence is an antigen-binding fragment of an anti-CD20 antibody.

In some embodiments, the antigen-binding domain comprising an anti-CD20 amino acid sequence is a single chain variable fragment (scFv).

In some embodiments, the antigen-binding domain comprising an anti-CD20 amino acid sequence comprises a heavy chain variable region (VH) that comprises a first heavy chain complementarity determining region (HCDR1) having the sequence SYNMH (SEQ ID NO: 803), a second heavy chain complementarity determining region (HCDR2) having the sequence AIYPGNGDTSYNQKFKG (SEQ ID NO: 804), and a third heavy chain complementarity determining region (HCDR3) having the sequence SNYYGSSYWFFDV (SEQ ID NO: 805).

In some embodiments, the antigen-binding domain comprising an anti-CD20 amino acid sequence comprises a light chain variable region (VL) that comprises a first light chain complementarity determining region (LCDR1) having the sequence RASSSVNYMD (SEQ ID NO: 800), a second light chain complementarity determining region (LCDR2) having the sequence ATSNLAS (SEQ ID NO: 801), and a third light chain complementarity determining region (LCDR3) having the sequence QQWSFNPPT (SEQ ID NO: 802).

In some embodiments, the antigen-binding domain comprising an anti-CD20 amino acid sequence comprises a VH comprising a sequence having at least 90%, 95%, 96%, 97%, 98%, 99%, or 100% sequence identity to the sequence EVQLQQSGAELVKPGASVKMSCKASGYTFTSYNMHWVKQTPGQGLEWIGAIYPGNG DTSYNQKFKGKATLTADKSSSTAYMQLSSLTSEDSADYYCARSNYYGSSYWFFDVWG AGTTVTVSS (SEQ ID NO: 585) and a VL comprising a sequence having at least 90%, 95%, 96%, 97%, 98%, 99%, or 100% sequence identity to the sequence DIVLTQSPAILSASPGEKVTMTCRASSSVNYMDWYQKKPGSSPKPWIYATSNLASGVPA RFSGSGSGTSYSLTISRVEAEDAATYYCQQWSFNPPTFGGGTKLEIK (SEQ ID NO: 537).

In some embodiments, the antigen-binding domain comprising an anti-CD20 amino acid sequence comprises a VH having the sequence EVQLQQSGAELVKPGASVKMSCKASGYTFTSYNMHWVKQTPGQGLEWIGAIYPGNG DTSYNQKFKGKATLTADKSSSTAYMQLSSLTSEDSADYYCARSNYYGSSYWFFDVWG AGTTVTVSS (SEQ ID NO: 585) and a VL having the sequence DIVLTQSPAILSASPGEKVTMTCRASSSVNYMDWYQKKPGSSPKPWIYATSNLASGVPA RFSGSGSGTSYSLTISRVEAEDAATYYCQQWSFNPPTFGGGTKLEIK (SEQ ID NO: 537).

In some embodiments, the VH and VL are linked by a linker comprising the sequence GSTSGGGSGGGSGGGGSS (SEQ ID NO: 806).

In some embodiments, the antigen-binding domain comprising an anti-CD20 amino acid sequence comprises a sequence having at least 90%, 95%, 96%, 97%, 98%, 99%, or 100% sequence identity to the sequence DIVLTQSPAILSASPGEKVTMTCRASSSVNYMDWYQKKPGSSPKPWIYATSNLASGVPA RFSGSGSGTSYSLTISRVEAEDAATYYCQQWSFNPPTFGGGTKLEIKGGGSSRSSSSGGG GSGGGGEVQLQQSGAELVKPGASVKMSCKASGYTFTSYNMHWVKQTPGQGLEWIGA IYPGNGDTSYNQKFKGKATLTADKSSSTAYMQLSSLTSEDSADYYCARSNYYGSSYWF FDVWGAGTTVTVSS (SEQ ID NO: 467).

In some embodiments, the antigen-binding domain comprising an anti-CD20 amino acid sequence comprises a sequence having at least 90%, 95%, 96%, 97%, 98%, 99%, or 100% sequence identity to the sequence EVQLQQSGAELVKPGASVKMSCKASGYTFTSYNMHWVKQTPGQGLEWIGAIYPGNG DTSYNQKFKGKATLTADKSSSTAYMQLSSLTSEDSADYYCARSNYYGSSYWFFDVWG AGTTVTVSSGSTSGGGSGGGSGGGSSDIVLTQSPAILSASPGEKVTMTCRASSSVNYMD WYQKKPGSSPKPWIYATSNLASGVPARFSGSGSGTSYSLTISRVEAEDAATYYCQQWSF NPPTFGGGTKLEIK (SEQ ID NO: 819)

In some embodiments, each of the transmembrane domains and the cytoplasmic domain of the first and second fusion proteins comprise a KIR2DS2 domain. In some embodiments, the nucleic acid sequence encoding the KIR2DS2 domain is codon optimized.

In some embodiments, each of the first fusion protein and the second fusion protein comprise a DAP12 association sequence, the DAP12 association sequence of the first fusion protein comprises the amino acid sequence of SEQ ID NOS: 753 or a functional variant thereof that comprises at least about 75% sequence identity SEQ ID NOS: 753, and the DAP12 association sequence of the second fusion protein comprises the amino acid sequence of SEQ ID NO: 753 or a functional variant thereof that comprises at least about 75% sequence identity to a respective one of SEQ ID NOS: 753.

In some embodiments, the transmembrane domain of the first nucleic acid sequence associates with DAP12 if the antigen-binding domain is associated with CD19.

In some embodiments, the transmembrane domain of the second nucleic acid sequence associates with DAP12 if the antigen-binding domain is associated with CD20.

In some embodiments, the first fusion protein and second fusion protein comprise a signal peptide. In some embodiments, the signal peptide is from CD8α. In some embodiments, the nucleic acid sequence encoding the signal peptide is codon optimized. In some embodiments, the signal peptide is encoded by nucleic acid sequence comprising SEQ ID NO: 699.

In some embodiments, the first nucleic acid sequence and the second nucleic sequence are encoded on the same nucleic acid molecule.

In some embodiments, the first nucleic acid sequence and the second nucleic acid sequence are separated by a sequence encoding a P2A peptide, E2A peptide, T2A peptide, or a furin sequence.

In some embodiments, the first nucleic acid sequence, the second nucleic sequence, and the third nucleic acid sequence are encoded on the same nucleic acid molecule.

In some embodiments, the first nucleic acid sequence, the second nucleic acid sequence, and the third nucleic acid sequence are each separated by a sequence encoding a P2A peptide, E2A peptide, T2A peptide, or a furin sequence.

In some embodiments, the single recombinant nucleic acid molecule comprises, from 5′ to 3′, a sequence encoding DAP12; a sequence encoding a T2A ribosome skipping peptide; a sequence encoding the first fusion protein; a sequence encoding a P2A ribosome skipping peptide; and a sequence encoding the second fusion protein.

In some embodiments, the single recombinant nucleic acid molecule comprises a sequence encoding the amino acid sequence of SEQ ID NO: 720.

Also provided herein is a composition comprising one or more polypeptides encoded by the one or more recombinant nucleic acid molecules of the composition of the present disclosure.

Also provided herein is a cell comprising the one or more recombinant nucleic acid molecules of the composition of the present disclosure or the one or more polypeptides of the present disclosure.

In some embodiments, the cell is an immune cell. In some embodiments, the immune cell is a T cell, a B cell, a natural killer (NK) cell, a macrophage, a dendritic cell, a monocyte, or a granulocyte.

In some embodiments, the cell is a T cell. In some embodiments, the T cell is a CD8-positive T cell, a CD4-positive T cell, a regulatory T cell (Treg), a cytotoxic T cell (CTL), a central memory T cell (TCM), an effector memory T cell (TEM), a tissue-resident memory T cell (TRM), a stem cell-like memory T cell (TSCM) or a tumor infiltrating lymphocyte (TIL).

Also provided herein is a population of cells comprising the one or more recombinant nucleic acid molecules of the composition of the present disclosure or the one or more polypeptides of the present disclosure.

Also provided herein is a pharmaceutical composition comprising the one or more recombinant nucleic acid molecules of the present disclosure and a pharmaceutically acceptable carrier.

Also provided herein is a pharmaceutical composition comprising the one or more polypeptides of the present disclosure and a pharmaceutically acceptable carrier.

Also provided herein is a pharmaceutical composition comprising the cell of the present disclosure and a pharmaceutically acceptable carrier.

Also provided herein is a pharmaceutical composition comprising the population of cells of the present disclosure and a pharmaceutically acceptable carrier.

Also provided herein is a method of treatment of a B cell-mediated disorder in a subject in need thereof comprising administering to the subject a pharmaceutical composition of the present disclosure. In some embodiments, the method is free of CD28 stimulation.

In some embodiments, the method further comprises a step administering a chemotherapeutic agent simultaneously with, before, or after administration of the pharmaceutical composition.

In some embodiments, the B cell-mediated disorder is a hyperproliferative disorder.

In some embodiments, the B cell-mediated disorder is a B cell lymphoma.

Also provided herein is a recombinant nucleic acid molecule comprising from 5′ to 3′: a. a nucleic acid sequence encoding a DAP12 polypeptide, including an extracellular, transmembrane, and cytoplasmic domain; b. a nucleic acid sequence encoding a T2A ribosomal skip peptide; c. a nucleic acid sequence encoding an anti-CD19 antigen binding domain operably linked to a KIR2DS2-derived transmembrane, short extracellular domain, and intracellular domain d. a nucleic acid sequence encoding a P2A ribosomal skip peptide; and e. a nucleic acid sequence encoding an anti-CD20 antigen binding domain operably linked to a KIR2DS2-derived transmembrane, long extracellular domain, and intracellular domain.

Also provided herein is a polypeptide comprising, in order from the amino terminus to the carboxy terminus: (a) a DAP12 polypeptide, including an extracellular, transmembrane, and cytoplasmic domain; (b) a T2A ribosomal skip peptide; (c) an anti CD19 antigen binding domain operably linked to a KIR2DS2 derived transmembrane, short extracellular domain, and intracellular domain; (d) a P2A ribosomal skip peptide; and (e) an anti CD20 antigen binding domain operably linked to a KIR2DS2 derived transmembrane, long extracellular domain, and intracellular domain.

### BRIEF DESCRIPTION OF THE DRAWINGS

The following detailed description of embodiments will be better understood when read in conjunction with the appended drawings. For the purpose of illustrating the invention, there are shown in the drawings certain embodiments. It is understood, however, that the embodiments herein are not limited to the precise arrangements and instrumentalities shown. In the drawings:

FIGS. 1A and 1B illustrate that despite the success of CAR T therapy against CD19-positive hematological malignancies there is still high relapse rate. FIG. 1A, illustrates CAR constructs (Nature Reviews Clinical Oncology volume 20, pages 359-371 (2023)). FIG. 1B illustrates a PFS-Kaplan-Meier curve (Transplantation and Cellular Therapy 30 (2024) 77.e1 77.e15).

FIG. 2 illustrates an exemplary dual binder concept.

FIG. 3 shows that all dual binder candidates are effective at killing CD19+ target cells. 40,000 target cells (K562-CD19 GFP) at a 3:1 E:T ratio.

FIG. 4 shows limited efficacy of dual binder candidates at killing CD20+ target cells when mRNA electroporation is used to induce expression. 40,000 target cells (K562-CD20 GFP) at a 3:1 E:T ratio.

FIGS. 5A-5B shows overall killing of CD19 and CD20 target cells with mRNA electroporation of dual binders into T cells.

FIGS. 6A-6F show cytokine secretion after 24 hour co-culture with CD19/20 dual-positive, CD19+/CD20− or CD19−/CD20+ Raji target cells.

FIG. 7 shows surface expression of binders in T cells after lentivirus infection (Donor 1).

FIG. 8 shows surface expression of binders in T cells after lentivirus infection (Donor 2).

FIG. 9 illustrates donor 1 killing. Dual binder candidates effectively kill both CD19+ and CD20+ target cells. 20,000 target cells at a 3:1 E:T ratio.

FIG. 10 illustrates donor 1 killing. Dual binder candidates effectively kill both CD19+ and CD20+ target cells. 20,000 target cells at a 3:1 E:T ratio.

FIGS. 11A-11B show that dual-KIR effectively kills both CD19+ and CD20+ target cells (calculated by normalized target cell total number after 48 hours of co-culture).

FIG. 12 shows target cell kill calculated by specific lysis with CD19+ or CD20+ targets.

FIG. 13A-13L shows the elimination of sequence deletions due to lentiviral template switching and cryptic splicing. FIG. 13A shows an outline of constructs with the Leu16-KIRshort design, and spurious constructs generated from template switching. Identical regions (CD8α SP and KIR2DS2) in the initial dual binder designs (1) result in unintentional generation of single-KIR constructs (2,3) during lentiviral packaging. Construct (4) shows a codon optimized Leu16-KiIRshort design. FIG. 13B shows flow cytometry expression of DS191 and Leu16 in T cells electroporated with mRNA encoded by construct (1). Only one expressing population is observed. FIG. 13C shows flow cytometry expression of DS191 and Leu16 in T cells infected with lentivirus encoded with construct (1). Three populations are observed corresponding to construct (1) and template switching products (2) and (3). FIG. 13D shows flow cytometry expression of DS191 and Leu16 in T cells infected with lentivirus encoded with codon-optimized construct (4). FIG. 13E shows an outline of single-KIR constructs with the Leu16-KIRLong design, and spurious constructs generated from cryptic splicing. Construct (7) is cryptic splice product of (6). Also shown are the Leu16 KIRsShort design (5) and the codon optimized Leu16-KIRLong design (8). FIG. 13F shows flow cytometry expression of DAP12 and Leu16 in Leu16-KIRShort construct (5). FIG. 13G shows PCR amplification of genomic DNA from T cells infected with constructs (6) or (8) show presence of a truncated product corresponding to a cryptic splice event in Leu16-KIRLong, but not in Leu16-KIRLong-Opt. FIG. 13H shows flow cytometry expression of DAP12 and Leu16 in T cells infected with Leu16-KIRLong. Populations corresponding to full length (6) and cryptic splicing products (7) are indicated. FIG. 13I shows flow cytometry expression of DAP12 and Leu16 in T cells infected with Leu16-KIRLong-CodOpt showing presence of only full length construct (8). FIG. 13J shows an outline of dual-KIR constructs with the Leu16-KIRLong (9) or Leu16-KIRLong-Opt designs (11). Construct (10) is generated from cryptic splicing of construct (9). FIG. 13K shows flow cytometry expression of DS191 and Leu16 in T cells infected with DS191-Leu16-KIRLong lentivirus. Full length (9) and cryptic splicing (10) products highlighted. FIG. 13L shows flow cytometry expression of DS191 and Leu16 in T cells infected with DS191-Leu16-KIRLong-CodOpt lentivirus (11), with no spurious products observed.

FIGS. 14A-14D show increasing the linker length between Leu16 and the KIR2DS2 transmembrane domain improves anti-CD20 tumor lysis in vitro and in vivo. In FIG. 14A, data represents results from an in vitro luciferase killing assay (Promega, E1501). Original Leu16-KIRShort, a longer Leu16-(G4S)5-KIRShort with additional G4S linkers, a longer Leu16-KIRLong with added full length KIR2DS2 stem domain, negative control untransduced (UTD), DS191-KIRShort, and DAP12-T2A-DS191-KIRShort-P2A-Leu16-KIRShort-CodOpt T cells were co-cultured at a 1:1 Effector:Target (E:T) ratio with Raji wild-type (WT, CD19+CD20+), Raji CD19 KO cells, and Raji CD20 KO cells for 18-24 hours. After incubation, a luciferase killing assay was performed and cells were read out on a plate reader (Varioskan lux, Thermo Fisher Scientific). Two technical replicates are shown for each group. FIG. 14B shows immunocompromised NOD-SCID-IL2Rγc^{-/-} (NSG) mice were first engrafted with 5×10⁵ mixed Raji cells (75% WT (CD19+CD20+) cells and 25% CD19 KO (CD20+) cells) expressing click beetle green luciferase-GFP (Raji-CBG-GFP) by IV injection on Day 0. On Day 4, a baseline tumor measurement was collected. On Day 5, mice were treated IV with 1×10⁶ CAR⁺ DS191-KIRShort (n=8), Leu16-KIRShort (n=8), Leu16-KIRLong (n=8), and DAP12-T2A-DS191-KIRShort-P2A-Leu16-KIRShortCodOpt (n=8) T cells, plus a negative control untransduced (UTD, n=8) group derived from healthy donor, EN01000657. Burkitt's lymphoma progression was monitored by bioluminescent imaging (BLI) of ventral areas twice per week for 21 days. BLI was assessed by imaging anesthetized mice using an Ami HT and Aura imaging software v4.5.0, following intraperitoneal injection of 150 mg/kg D-luciferin in groups of up to 5 mice at the same relative time point after luciferin injection. FIG. 14C shows the area under the curve (AUC) that was calculated from BLI data to represent overall tumor control. FIG. 14D shows photon emission from mixed Raji-CBG-GFP tumors in individual mice in each treatment group.

One-way analysis of variance (ANOVA) was conducted, and between-group comparisons were performed by a post hoc Tukey's HSD test. Significant differences were indicated as *p<0.05, **p<0.01, ***p<0.001, ****p<0.0001.

FIGS. 15A-15I show DAP12-T2A-DS191-KIRShort-P2A-Leu16-KIRLong-CodOpt shows superior performance compared to DAP12-T2A-DS191-KIRShort-P2A-Leu16-KIRShort-CodOpt, and reduced cytokines compared to Hu19-CD28z-huCD20-41BBz (Lam, et al. Mol Ther Oncolytics. 2023 Jul. 19; 30:132-149) in vitro. FIG. 15A shows T-cells overexpressing Leu16-KIRLong and Leu16-KIRLong-CodOpt single binders and DAP12-T2A-DS191-KIRShort-P2A-Leu16-KIRLong-CodOpt dual binder were co-cultured with Raji target cells (WT, CD19KO, CD20KO) at an E:T ratio of 3:1 for 24 h. Specific lysis was assessed by bioluminescence, with normalization to UTD+Target cell control wells. FIG. 15B shows co-culture of T cell transduced with single KIR (DS191-KIRShort, Leu16-KIRLong), dual KIR (DAP12-T2A-DS191-KIRShort-P2A-Leu16-KIRLong-CodOpt; DAP12-T2A-DS191-KIRShort-P2A-Leu16-KIRShort-CodOpt) or the CD3z-based bicistronic CAR huCD19-CD28z-huCD20-41BBz were co-culture with Raji target v cells (WT, CD19KO, CD20KO) at an E:T ratio of 3:1 for 24 h and analyzed by bioluminescence. FIGS. 15C-15H show T-cells expressing single KIR (DS191-KIRShort, Leu16-KIRLong), dual KIR (DAP12-T2A-DS191-KIRShort-P2A-Leu16-KIRLong-CodOpt; DAP12-T2A-DS191-KIRShort-P2A-Leu16-KIRShort-CodOpt) or the CD3z-based bicistronic CAR huCD19-CD28z-huCD20-41BBz were co-cultured with K562 target cells (WT, CD200E, CD19OE) at an E:T ratio of 3:1 for 72 h.) Incucyte target cell #curves for K562 WT is shown in FIG. 15C. Incucyte target cell #curves for CD200E is shown in FIG. 15D. Incucyte target cell #curves for CD19OE is shown in FIG. 15E, normalized to time 0. Area under the curves over 72 hours was assessed for each T cell construct co-cultured with K562 WT (FIG. 15F). K562 CD200E (FIG. 15G) or K562 CD19OE (FIG. 15H) target cells. FIG. 15I shows cytokines (IL2, TNFα, IFNγ) were assessed for the co-cultures evaluated in FIGS. 15G-15I. All results show n=3 technical replicates for one healthy donor.

FIGS. 16A-16G show DAP12-T2A-DS191-KIRShort-P2A-Leu16-KIRLong-CodOpt shows superior performance compared to DAP12-T2A-DS191-KIRShort-P2A-Leu16-KIRShort-CodOpt, and reduced cytokines compared to Hu19-CD28z-huCD20-41BBz in vivo. FIG. 16A shows immunocompromised NOD-SCID-IL2Rγc−/− (NSG) mice were first engrafted with 5×10⁵ mixed Raji cells (75% WT (CD19+CD20+) cells and 25% CD19 KO (CD20+) cells) expressing click beetle green luciferase-GFP (Raji-CBG-GFP) by IV injection on Day 0. On Day 4, a baseline tumor measurement was collected. On Day 5, mice were treated IV with 1×10⁶ CAR+ DS191-KIRShort (n=8), Leu16-KIRLong-CodOpt (n=8), DAP12-T2A-DS191-KIRShort-P2A-Leu16-KIRLongCodOpt (n=8), DAP12-T2A-DS191-KIRShort-P2A-Leu16-KIRShortCodOpt (n=8), and Hu19-CD28z-huCD20-41BBz (n=8) T cells, plus a negative control untransduced (UTD, n=8) group derived from healthy donor, ND727. Burkitt's lymphoma progression was monitored by bioluminescent imaging (BLI) of ventral areas twice per week for 18 days. BLI was assessed by imaging anesthetized mice using an Ami HT and Aura imaging software v4.5.0, following intraperitoneal injection of 150 mg/kg D-luciferin in groups of up to 5 mice at the same relative time point after luciferin injection. Blood was taken and spun for plasma in order to measure cytokine levels at Day 8. Whole blood collected on Day 8 was also used to measure circulating peripheral T cells prior to spinning for plasma. FIG. 16B shows mouse body weights were tracked twice weekly throughout the duration of the experiment. FIG. 16C shows photon emission from mixed Raji-CBG-GFP tumors. FIG. 16D shows the area under the curve (AUC) that was calculated from BLI data to represent overall tumor control. FIG. 16E shows photon emission from mixed Raji-CBG-GFP tumors in individual mice in each treatment group. FIG. 16F shows plasma cytokine levels were measured by Meso Scale Discovery at Day 8 post-tumor injection. FIG. 16G shows circulating peripheral T cells were measured on Day 8 post-tumor injection by collecting whole blood retroorbitally and adding 20 μL to CountBright™ Plus Ready Tubes (Invitrogen). The whole blood was blocked with TruStain FcX™ (anti-mouse CD16/32) (BioLegend) then stained with PE anti-human CD3 (BioLegend) and Alexa Fluor 700 anti-mouse CD45 (BioLegend), followed by red blood cell (RBC) lysis eBioscience™ 1×RBC Lysis Buffer (Invitrogen) and run on the Novocyte Penteon (Agilent). One-way analysis of variance (ANOVA) was conducted, and between-group comparisons were performed by a post hoc Tukey's HSD test. Significant differences were indicated as **p<0.01, ***p<0.001, ****p<0.0001.

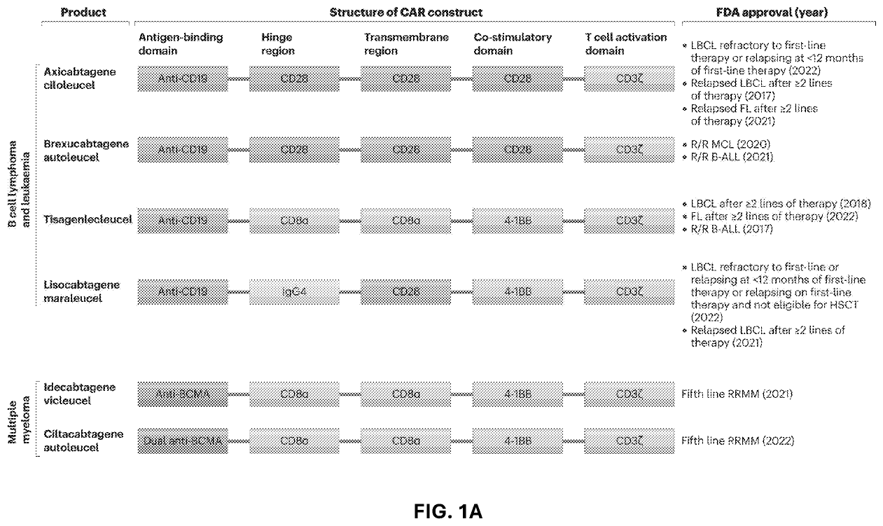

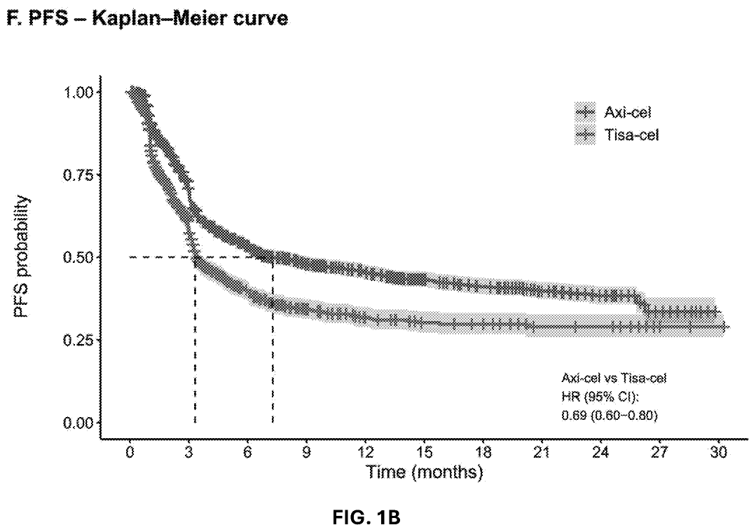

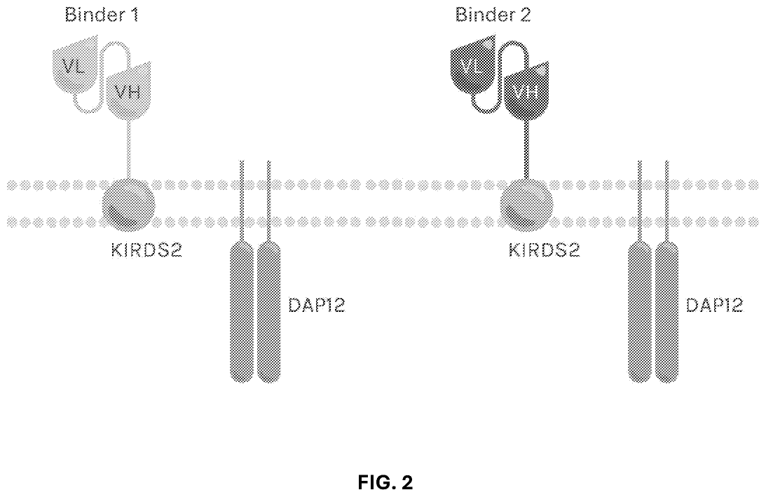

### DETAILED DESCRIPTION

The present disclosure provides compositions and methods that overcome key limitations of existing CAR T and CAR NK cell therapies by employing dual KIR based chimeric antigen receptors capable of independently targeting CD19 and CD20 while leveraging native DAP12 mediated signaling. As demonstrated herein, the disclosed constructs deliver multiple unexpected advantages, including the elimination of lentiviral template switching artifacts through codon optimization strategies, the removal of cryptic splice sites to ensure exclusive full-length expression, and the incorporation of optimized linker architectures that dramatically enhance CD20 directed cytotoxicity. Moreover, the dual KIR designs described in this application achieve potent and durable anti-tumor activity against heterogeneous and antigen escape B cell populations while simultaneously producing significantly reduced pro inflammatory cytokine release relative to conventional bicistronic CD3 (based CAR platforms. Collectively, these innovations provide anti-CD19/anti-CD20 dual targeted safety enhanced immune effector cell therapies with improved manufacturing robustness, superior functional performance, and a reduced risk of cytokine mediated toxicities in vivo.

Certain terminology is used in the following description for convenience only and is not limiting. The words “right,” “left,” “top,” and “bottom” designate directions in the drawings to which reference is made.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” as used herein when referring to a measurable value such as an amount, a temporal duration, and the like, is meant to encompass variations of 20% from the specified value, as such variations are appropriate to perform the disclosed methods. In some embodiments, a specified value encompasses ±10%, ±5%, ±1%, or ±0.1% from the specified value, as such variations are appropriate to perform the disclosed methods. For recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6, 9, and 7.0 are explicitly contemplated.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

The term “KIR polypeptide” refers to killer-cell immunoglobulin-like receptors and functional variants thereof.

KIR-CAR, as that term is used herein, refers to a CAR which shares functional and structural properties with a KIR.

The term “Chimeric Antigen Receptor,” a “CAR,” or a “CAR molecule” refers to a set of polypeptides, two in some embodiments, which when in an immune effector cell, provides the cell with specificity for a target cell and with intracellular signal generation. In some embodiments, a CAR comprises at least an extracellular antigen binding domain and a transmembrane domain. In some embodiments, a CAR comprises a cytoplasmic signaling domain (also referred to herein as “an intracellular signaling domain”) comprising a functional signaling domain derived from a stimulatory molecule and/or costimulatory molecule as defined below. In some embodiments, the set of polypeptides are contiguous with each other, e.g., are in the same polypeptide chain, e.g., comprise a chimeric fusion protein. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. A CAR, in some embodiments, is a chimeric polypeptide that shares structural and functional properties with a cell immune-function receptor or adaptor molecule, from e.g., a T cell or an NK cell. Upon binding to cognate antigen, a CAR can activate or inactivate the cytotoxic cell in which it is disposed or modulate the cell's antitumor activity or otherwise modulate the cells immune response.

As used herein, the term “CAR” comprises an antigen binding domain, an intracellular signaling domain, and optionally one or more costimulatory domains.

“Intracellular signaling domain”, as used herein, refers to a polypeptide sequence that is a component of a larger integral membrane protein. This polypeptide sequence, through regulated interactions with other cellular proteins, is capable of stimulating or inhibiting immune cell functions such as lytic granule release, cytokine production, or proliferation.

In some embodiments, the stimulatory molecule is the zeta chain associated with the T cell receptor complex. In some embodiments, the stimulatory molecule is a DAP12 polypeptide.

In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-IBB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

In some embodiments, a CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain, and an intracellular signaling domain comprising at least two functional signaling domains derived from one or more costimulatory molecule(s) and a functional signaling domain derived from a stimulatory molecule.

In some embodiments, a CAR comprises a leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., an scFv) during cellular processing and localization of the CAR to the cellular membrane.

The term “signaling domain” refers to the functional portion of a protein which acts by transmitting information within the cell to regulate cellular activity via defined signaling pathways.

As used herein, the terms “B cell-mediated disorder” refers to mammalian disorders in which pathologies are caused by dysfunctional or hyperfunctional B cells. In some embodiments, the B cell-mediated disorder is a B cell-mediated hyperproliferative disorder, such as non-Hodgkin's lymphoma (NHL) and Hodgkin's lymphoma (HL), B cell leukemia, Burkitt's lymphoma or diffuse large B-cell lymphoma.

As used herein, the term “binding domain” refers to a protein, e.g., an immunoglobulin chain or fragment thereof, comprising at least one domain that associates to or binds to a target sequence. In some embodiments, the binding domain is an immunoglobulin variable domain sequence. The term “binding domain” (also referred to herein as “antibody molecule”) encompasses antibodies and antibody fragments. In some embodiments, if we use the terms “anti-CD19” or “anti-CD20,” the terms are used to designate a binding domain that associates with or binds to its target, in the aforementioned example, CD19 or CD20, respectively. In some embodiments an antibody molecule or antibody fragment is a multi-specific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multi-specific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the binding domain comprises a CDR or a variable light chain, a variable heavy chain, or both a variable light and heavy chain, whereby, if the binding domain comprises a variable light and/or heavy chain the light and/or heavy chains comprise a CDR sequence that associates with or binds to the target sequence. In some embodiments, the amino acids of the disclosure bind to either CD19 or CD20 and comprise at least one or a combination of the CDR sequences identified in the disclosed Tables.

As used herein, a cleavable linker sequence refers to a ribosomal skip site or an amino acid sequence that is subject to post-translational cleavage.

The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and one or more isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein, an scFv may have the VL and VH variable regions in either order, e.g., with respect to the N-terminal and C-terminal ends of the polypeptide, the scFv may comprise VL-linker-VH or may comprise VH-linker-VL.

The term “complementarity determining region” or “CDR,” as used herein, refers to the sequences of amino acids within antibody variable regions which confer antigen specificity and binding affinity. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “IMGT”). Under IMGT, the CDR regions of an antibody can be determined using the program IMGT/DomainGap Align.

A portion of a KIR CAR herein may comprise an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further embodiment, the CAR comprises an antibody fragment that comprises an scFv.

“Fully human” refers to an immunoglobulin, such as an antibody, where the whole molecule is of human origin or consists of an amino acid sequence identical to a human form of the antibody.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (λ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleic acid sequence or a partial nucleic acid sequence encoding a protein that elicits an immune response therefore encodes an “antigen.” Furthermore, the skilled artisan will understand that an antigen need not be encoded solely by a full length nucleic acid sequence of a gene. It is readily apparent that the present disclosure includes, but is not limited to, the use of partial nucleic acid sequences of more than one gene and that these nucleic acid sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a tumor sample, a cell or a fluid with other biological components.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

As used herein, the term “autologous” is meant to refer to any material derived from the same individual to which it is later to be re-introduced into the individual.

The term “xenogeneic” refers to a graft derived from an animal of a different species.

The term “auto-antigen” means any self-antigen that is recognized by the immune system as if it were foreign. Auto-antigens comprise, but are not limited to, cellular proteins, phosphoproteins, cellular surface proteins, cellular lipids, nucleic acids, glycoproteins, including cell surface receptors.

The term “combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative; e.g. synergistic, effect. The single components may be packaged in a kit or separately. One or both of the components (e.g., powders or liquids) may be reconstituted or diluted to a desired dose prior to administration. The terms “co-administration” or “combined administration” or the like are meant to encompass administration of the selected combination partner to a single subject in need thereof (e.g. a patient). In some embodiments, “co-administration” or “combined administration” or the like includes administration at one or both of by the same route or at the same time. In some embodiments, “co-administration” or “combined administration” or the like are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present disclosure and a combination partner, are both administered to a patient simultaneously in the form of a single entity or dosage. The term “non-fixed combination” means that the therapeutic agents, e.g. a compound of the present disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “hyperproliferative disorder” refers to a disease or disorder characterized by abnormal proliferation, abnormal growth, abnormal senescence, abnormal quiescence, or abnormal removal of cells in an organism, and includes all forms of hyperplasias, neoplasias, and cancer. In some embodiments, a hyperproliferative disease is a cancer. In some embodiments, a hyperproliferative disease is a cancer derived from the gastrointestinal tract or urinary system. In some embodiments, a hyperproliferative disease is a cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus. In some embodiments, a hyperproliferative disease is a cancer chosen from: lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), neoplasms of the central nervous system (e.g., primary CNS lymphoma, spinal axis tumors, brain stem gliomas or pituitary adenomas), non-Hodgkin Lymphoma, chronic lymphocytic leukemia, Burkitt lymphoma, chronic lymphocytic leukemia (CLL), acute lymphoblastic leukemia (ALL), B-cell lymphoma, and B-cell leukemia. In some embodiments, hyperproliferative disorder refers to those in Alaggio, R. et al., cited above and incorporated herein by reference.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid tumors. As used herein, the term “cancer” or “tumor” includes premalignant, as well as malignant cancers and tumors.

The term “autoimmune disease” as used herein is as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, unless otherwise specified, the terms “prevent,” “preventing” and “prevention” refer to an action that occurs before the subject begins to suffer from the condition, or relapse of the condition. Prevention need not result in a complete prevention of the condition; partial prevention or reduction of the condition or a symptom of the condition, or reduction of the risk of developing the condition, is encompassed by this term.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

“Derived from,” as that term is used herein, indicates a relationship between a first and a second molecule. It generally refers to structural similarity between the first molecule and a second molecule and does not connote or include a process or source limitation on a first molecule that is derived from a second molecule.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within polypeptide herein can be replaced with other amino acid residues from the same side chain family and the altered polypeptide can be tested using the functional assays described herein.

The term “stimulation” refers to a primary response induced by binding of a stimulatory molecule with its cognate ligand thereby mediating a signal transduction event. A non-limiting example of stimulation includes signal transduction via a transmembrane KIR domain complexing with a transmembrane DAP12 domain. Stimulation can mediate altered expression of certain molecules.

The term “stimulatory molecule,” refers to a molecule expressed by an immune cell, e.g., T cell, NK cell, or B cell, that provides the cytoplasmic signaling sequence(s) that regulates activation of the immune cell in a stimulatory way for at least some embodiment of the immune cell signaling pathway. In some embodiments, the signal is a primary signal that is initiated by, for instance, binding of a KIR-CAR with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloid-derived phagocytes.

“Immune effector function or immune effector response,” as that term is used herein, refers to function or response, e.g., of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and co-stimulation are examples of immune effector function or response.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Accession No. AAA62478.2, or the equivalent residues from a non-human species, e.g., mouse, rodent, monkey, ape and the like. In some embodiments, the “4-1BB costimulatory domain” is KRGRKKLLYIFKQPFMRPVQTTQEEDGCSCRFPEEEEGGCEL (SEQ ID NO: 665) or the equivalent residues from a non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleic acid sequence of which is identical to the mRNA sequence and is usually provided in sequence listings, and the non-coding strand, used as the template for transcription of a gene or cDNA, can be referred to as encoding the protein or other product of that gene or cDNA.

A “nucleic acid molecule”, as referred to herein, may be DNA or RNA, naturally-occurring or synthetic, or any combination thereof, constructed in such a way that it comprises a nucleic acid sequence to be translated by the machinery of the cells of a host organism. Thus, natural nucleic acids may be modified, for example to increase the stability of an expressible nucleic acid sequence comprising DNA and/or RNA. DNA or RNA may be modified in order to improve nuclease resistance. For example, known modifications for ribonucleotides include 2′-0-methyl, 2′-fluoro, 2′-NH2, and 2 —O-allyl. Modified nucleic acids may comprise chemical modifications which have been made in order to increase the in vivo stability of the nucleic acid, enhance or mediate the delivery of a nucleic acid sequence, or reduce the clearance rate from the body. Examples of such modifications include chemical substitutions at the ribose and/or phosphate and/or base positions of a given RNA sequence. See, for example, WO 92/03568; U.S. Pat. No. 5,118,672; Hobbs et al., (1973) Biochemistry 12:5138; Schauer et al., (1977) Nucleic Acids Res. 4: 1933; Schibaharu et al., (1987) Nucleic Acids Res. 15:4403; Pieken et al., (1991) Science 253:314.

Unless otherwise specified, a “nucleic acid sequence encoding an amino acid sequence” includes all nucleic acid sequences that are degenerate versions of each other and that encode the same amino acid sequence. The phrase nucleic acid sequence that encodes a protein or an RNA may also include introns to the extent that the nucleic acid sequence encoding the protein may in some versions contain one or more introns.

An “effective amount” as used herein, means an amount that is therapeutically effective, prophylactically effective, or both.

The term “therapeutic” as used herein means a treatment. The term “prophylactic” as used herein means prevention. To be “therapeutically effective” means suppression, diminution, remission, or eradication of at least one sign or symptom of a disease or disordered state. To be prophylactically effective means preventing a disease or disordered state.

The term “therapeutically effective amount” refers to the amount of the subject compound or composition that will elicit the biological or medical response of a tissue, system, or subject that is being sought by the researcher, veterinarian, medical doctor or other clinician. The term “therapeutically effective amount” includes that amount of a compound or composition that, when administered, is sufficient to alleviate to some extent, one or more of the signs or symptoms of the disease or disordered state being treated. The therapeutically effective amount will vary depending on the compound or composition, the disease and its severity and the age, weight, etc., of the subject to be treated.

The term “prophylactically effective amount” refers to the amount of the subject compound or composition that will elicit the biological or medical response of a tissue, system, or subject that is being sought by the researcher, veterinarian, medical doctor or other clinician in preventing a disease or disordered state. The term “prophylactically effective amount” includes that amount of a compound or composition that, when administered, is sufficient to prevent development of to some extent, one or more of the signs or symptoms of a disease or disordered state. The prophylactically effective amount will vary depending on the compound, the disease and its severity and the age, weight, etc., of the subject to be treated.

The term “endogenous” refers to any material from or produced inside an organism, cell, tissue or system.

The term “exogenous” refers to any material introduced from or produced outside an organism, cell, tissue or system.

The term “expression” refers to the transcription of a particular nucleic acid sequence driven by a promoter and/or translation of the particular nucleic acid sequence or transcripts thereof.

The term “transfer vector” refers to a composition of matter that comprises an isolated nucleic acid and which can be used to deliver the isolated nucleic acid to the interior of a cell.

Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, LNPs, mRNA LNPs, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleic acid sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The “percent identity” of two polynucleotide or two polypeptide sequences is determined by comparing the sequences using the GAP computer program (a part of the GCG Wisconsin Package, version 10.3 (Accelrys, San Diego, Calif)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences or amino acid sequences would occur by chance. For example, a nucleic acid is considered similar to another if the smallest sum probability in comparison of the test nucleic acid to the other nucleic acid is less than about 1, less than about 0.1, less than about 0.01, or less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “hybridizable” to another polynucleotide if the two polynucleotides can hybridize to one another under moderately stringent conditions. Thus, a polynucleotide can be hybridizable to another polynucleotide without being its complement. In some embodiments, polynucleotides are hybridizable under high stringency conditions.

The term “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of I 0), are matched or homologous, the two sequences are 90% homologous.

“Murine” refers to mice or rats. For example, a murine antibody or fragment thereof contains the sequence of an antibody or fragment thereof that is isolated from a murine animal, e.g., mouse or rat.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The terms “functional fragment” means any portion of a polypeptide or nucleic acid sequence from which the respective full-length polypeptide or nucleic acid relates that is of a sufficient length and has a sufficient structure to confer a biological effect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based (such wild-type or full-length sequences “reference sequences” or each individually a “reference sequence”). In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type human sequence upon which the sequence is derived. In some embodiments, the functional fragment may retain about 85%, 80%, 75%, 70%, 65%, or 60% sequence identity to the wild-type sequence upon which the sequence is derived.

By “fragment” is meant a portion of a polypeptide or nucleic acid molecule. This portion contains, in some embodiments, at least about 5%, 10%, 20%, 30%, 40%, 50%, 60%, 70%, 80%, or about 90% of the entire length of the reference nucleic acid molecule or polypeptide.

The term “functional variant thereof” refers to a nucleic acid or peptide varying from a reference sequence but retaining biological activity compared to a nucleic acid or peptide having the reference sequence. In some embodiments, the biological activity is measured by a functional assay disclosed herein. In some embodiments, a “functional variant thereof” has at least about 70%, 75%, 80%, 85%, 90%, 91%, 92%, 93%, 94%, 95%, 96%, 97%, 98%, 99% or more sequence identity to a reference sequence. In some embodiments, a “functional variant thereof” has a sequence identity to a reference sequence between any two of about 70%, about 75%, about 80%, about 85%, about 90%, about 91%, about 92%, about 93%, about 94%, about 95%, about 96%, about 97%, about 98%, about 99%, or about 100% sequence identity. In some embodiments, a “functional variant thereof” has a sequence identity of about 70%, 75%, 80%, 85%, 90%, 91%, 92%, 93%, 94%, 95%, 96%, 97%, 98%, or 99% to a stated reference sequence.

“Variant” as the term is used herein, is a nucleic acid sequence or a peptide sequence that differs in sequence from a reference nucleic acid sequence or peptide sequence, respectively. In some embodiments, a variant is a functional variant in that it retains essential biological properties of the reference nucleic acid molecule or amino acid sequence. Changes in the sequence of a nucleic acid variant may not alter the amino acid sequence of a peptide encoded by the reference nucleic acid, or may result in amino acid substitutions, additions, deletions, fusions and truncations. A functional variant includes a functional fragment. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring, such as an allelic variant, or can be a variant that is not known to occur naturally. Non-naturally occurring variants of nucleic acids and peptides may be made by mutagenesis techniques or by direct synthesis. In some embodiments, a nucleic acid sequence variant has at least about 70%, about 75%, about 80%, about 85%, about 86%, about 87%, about 88%, about 89%, about 90%, about 91%, about 92%, about 93%, about 94%, about 95%, about 96%, about 97%, about 98%, or about 99% sequence identity to a reference nucleic acid molecule herein. In some embodiments, an amino acid sequence variant has at least about 70%, about 75%, about 80%, about 85%, about 86%, about 87%, about 88%, about 89%, about 90%, about 91%, about 92%, about 93%, about 94%, about 95%, about 96%, about 97%, about 98%, or about 99% sequence identity to a reference amino acid sequence herein.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence that is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one that expresses the gene product in a tissue specific manner.

The term “constitutive” promoter refers to a nucleic acid sequence that, when operably linked with a polynucleotide that encodes or specifies a gene product, causes the gene product to be produced in a cell under most or all physiological conditions of the cell.

The term “inducible” promoter refers to a nucleic acid sequence that, when operably linked with a polynucleotide that encodes or specifies a gene product, causes the gene product to 10 be produced in a cell substantially only when an inducer that corresponds to the promoter is present in the cell.

The term “tissue-specific” promoter refers to a nucleic acid sequence that, when operably linked with a polynucleotide that encodes or specifies a gene product, causes the gene product to be produced in a cell substantially only if the cell is a cell of the tissue type corresponding to the promoter.

The term “flexible polypeptide linker” or “linker” as used in the context of a scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser) (SEQ ID NO: 289), repeated n times where n is a positive integer equal to or greater than 1. For example, n=1, n=2, n=3. N=4, n=5 and n=6, n=7, n=8, n=9 and n=10. In some embodiments, the flexible polypeptide linker is (Gly4 Ser)3 (SEQ ID NO: 282). In some embodiments, the linkers include multiple repeats of (Gly2Ser), and (GlySer). In some embodiments, the polypeptide does not include a linker, e.g., (n=0). Also included within the scope of the invention are linkers described in WO2012/138475, incorporated herein by reference).

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNAses. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In some embodiments, the terms “treat”, “treatment” and “treating”-refer to the inhibition of the progression of a proliferative disorder, either physically by, e.g., stabilization of a discernible symptom, physiologically by, e.g., stabilization of a physical parameter, or both. In some embodiments, the terms “treat”, “treatment” and “treating” refer to the reduction or stabilization viral infection, number of cells infected with virus, or viral disorder symptoms.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a stimulatory tumor antigen) protein present in a sample, but which antibody or ligand does not substantially recognize or bind other molecules in the sample.

A subject “responds” to treatment if a parameter of a viral in the subject is retarded or reduced by a detectable amount, e.g., about 5%, 10%, 20%, 30%, 40%, 50%, 60%, 70%, 80%, 90% or more as determined by any appropriate measure, e.g., by symptoms. In one example, a subject responds to treatment if the subject experiences a life expectancy extended by about 5%, 10%, 20%, 30%, 40%, 50% or more beyond the life expectancy predicted if no treatment is administered. In another example, a subject responds to treatment, if the subject has an increased disease-free survival, overall survival or increased time to progression. Several methods can be used to determine if a patient responds to a treatment including, for example, criteria provided by NCCN Clinical Practice Guidelines in Oncology (NCCN Guidelines®). For example, in the context of BALL, a complete response or complete responder, may involve one or more of: <5% BM blast, >1000 neutrophil/ANC (/μL). >100,000 platelets (/μL) with no circulating blasts or extramedullary disease (no lymphadenopathy, splenomegaly, skin/gum infiltration/testicular mass/CNS involvement), Trilineage hematopoiesis, and no recurrence for 4 weeks. A partial responder may involve one or more of >50% reduction in BM blast, >1000 neutrophil/ANC (/μL). >100,000 platelets (/μL). A non-responder can show disease progression, e.g., >25% in BM blasts. In some embodiments, a complete responder is defined as having 7% or greater CD27+CD45RO− cells in the CD8+ population. In some embodiments, the percent of CAR+ cells at pre-harvest levels distinguish responders (e.g., complete responders and partial responders) from non-responders (NR).

The term “relapse” as used herein refers to reappearance of a viral disorder after an initial period of responsiveness. The initial period of responsiveness may involve the level of symptoms or viral load falling below a certain threshold, e.g., below 20%, 1%, 10%, 5%, 4%, 3%, 2%, or 1%. The reappearance may involve the level symptoms or viral load rising above a certain threshold, e.g., above 20%, 1%, 10%, 5%, 4%, 3%, 2%, or 1%. In some embodiments, the initial period of 30 responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

“Regulatable chimeric antigen receptor (RCAR),” as that term is used herein, refers to a set of polypeptides, typically two in the simplest embodiments, which when in a RCARX cell, provides the RCARX cell with specificity for a target cell, typically a virus infected cell, and with regulatable intracellular signal generation or proliferation, which can optimize an immune effector property of the RCARX cell. An RCARX cell relies at least in part, on an antigen binding domain to provide specificity to a target cell that comprises the antigen bound by the antigen binding domain. In some embodiments, an RCAR includes a dimerization switch that, upon the presence of a dimerization molecule, can couple an intracellular signaling domain to the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In some embodiments, the first and second switch domains are the same as one another, e.g., they are polypeptides having the same primary amino acid sequence and are referred to collectively as a homodimerization switch. In some embodiments, the first and second switch domains are different from one another, e.g., they are polypeptides having different primary amino acid sequences and are referred to collectively as a heterodimerization switch. In some embodiments, the switch is intracellular. In some embodiments, the switch is extracellular. In some embodiments, the switch domain is a polypeptide-based entity, e.g., FKBP or FRB-based, and the dimerization molecule is small molecule, e.g., a rapalogue. In some embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In some embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In some embodiments, the dimerization molecule does not naturally occur in the subject or does not occur in concentrations that would result in significant dimerization. In some embodiments, the dimerization molecule is a small molecule, e.g., rapamycin or a rapalogue, e.g., RAD001.

The term “bioequivalent” refers to an amount of an agent other than the reference compound (e.g., RAD00I), required to produce an effect equivalent to the effect produced by the reference dose or reference amount of the reference compound (e.g., RAD001). In some embodiments the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In some embodiments, the effect is alteration of the ratio of PD-I positive/PD-I negative T cells, as measured by cell sorting. In some embodiments a bioequivalent amount or dose of an mTOR inhibitor is the amount or dose that achieves the same level of P70 S6 kinase inhibition as does the reference dose or reference amount of a reference compound.

In some embodiments, a bioequivalent amount or dose of an mTOR inhibitor is the amount or dose that achieves the same level of alteration in the ratio of PD-I positive/PD-I negative T cells as does the reference dose or reference amount of a reference compound.

The term “low, immune enhancing, dose” when used in conjunction with an mTOR inhibitor, e.g., an allosteric mTOR inhibitor, e.g., RAD00I or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In some embodiments, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In some embodiments, the low, immune enhancing, dose of mTOR inhibitor results in an increase in the number of naive T cells. In some embodiments, the low, immune enhancing, dose of mTOR inhibitor results in one or more of the following: an increase in the expression of one or more of the following markers: CD62L^{high} CD127^{high}, CD27+, and BCL2+, e.g., on memory T cells, e.g., memory T cell precursors; a decrease in the expression of KLRG1, e.g., on memory T cells, e.g., memory T cell precursors; and an increase in the number of memory T cell precursors, e.g., cells with any one or combination of the following characteristics: increased CD62L^{high} increased CD127^{high} increased CD27+, decreased KLRG1, and increased BCL2; wherein any of the changes described above occurs, e.g., at least transiently, e.g., as compared to a non-treated subject.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically 20 disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% sequence identity, and includes subranges such as 96-99%, 96-98%, 96-97%, 97-99%, 97-98% and 98-99% sequence identity. This applies regardless of the breadth of the range.

Adaptor molecule, as that term is used herein, refers to a polypeptide with a sequence that permits interaction with two or more molecules, and in embodiments, promotes activation or inactivation of a cytotoxic cell. E.g., in the case of DAP12, this comprises interactions with an activating KIR via charge interactions within the transmembrane domain and interactions with signaling molecules like ZAP70 or Syk via a phosphorylated ITAM sequence within the cytoplasmic domain.

Cytoplasmic and intracellular, as applied to adaptor molecules and signaling domains are used interchangeably herein.

Some embodiments herein comprise a novel CAR construct featuring two separate functional KIR-CARs. In some embodiments, a CAR construct herein reduces risk of mutual interference in target antigen binding.

In some embodiments, the disclosure provides a multicistronic CD19−CD20 KIR-CAR protein construct and corresponding nucleic acids encoding this construct are provided. In some embodiments, novel multicistronic CD19−CD20 KIR-CAR herein can target CD19+CD20−, CD19+/CD20low, CD19−CD20+, CD19lowCD20+, CD19+CD20+ B cell malignancies. In some embodiments, this dual-targeting strategy overcomes antigen escape, and improves the potency and durability of CAR T-cell therapy for treating B cell-mediated malignancies and autoimmune diseases.

### KIR-CAR General Description

Some embodiments herein include one or more aspect of KIR-CARs as discussed herein.

NK cell immune-function receptor (or NKR) refers to an endogenous naturally occurring transmembrane protein expressed in NK cells, which can engage with a ligand on an antigen presenting cell and modulate an NK cell immune-function response, e.g., it can modulate the cytolytic activity or cytokine secretion of the NK cell.

NK cells are mononuclear cells that develop in the bone marrow from lymphoid progenitors, and morphological features and biological properties typically include the expression of the cluster determinants (CDs) CD 16, CD56, and/or CD57; the absence of the alpha/beta or gamma/delta TCR complex on the cell surface; the ability to bind to and kill target cells that fail to express “self major histocompatibility complex (MHC)/human leukocyte antigen (HLA) proteins; and the ability to kill tumor cells or other diseased cells that express ligands for activating NK receptors. NK cells are characterized by their ability to bind and kill several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Upon activation by interferons and/or cytokines, NK cells mediate the lysis of tumor cells and of cells infected with intracellular pathogens by mechanisms that require direct, physical contacts between the NK cell and the target cell. Lysis of target cells involves the release of cytotoxic granules from the NK cell onto the surface of the bound target, and effector proteins such as perforin and granzyme B that penetrate the target plasma membrane and induce apoptosis or programmed cell death. Normal, healthy cells are protected from lysis by NK cells. NK cell activity is regulated by a complex mechanism that involves both stimulating and inhibitory signals.

Briefly, the lytic activity of NK cells is regulated by various cell surface receptors that transduce either positive or negative intracellular signals upon interaction with ligands on the target cell. The balance between positive and negative signals transmitted via these receptors determines whether or not a target cell is lysed (killed) by a NK cell. NK cell stimulatory signals can be mediated by Natural Cytotoxicity Receptors (NCR) such as NKp30, NKp44, and NKp46; as well as NKG2C receptors, NKG2D receptors, certain activating killer cell immunoglobulin-like receptors (KIRs), and other activating NK receptors (Lanier, Annual Review of Immunology 2005; 23:225-74). NK cell inhibitory signals can be mediated by receptors like Ly49, CD94/NKG2A, as well as certain inhibitory KIRs, which recognize major histocompatibility complex (MHC) class I molecules (Karre et al., Nature 1986; 319:675-8; Ohlen et al, Science 1989; 246:666-8).

These inhibitory receptors bind to polymorphic determinants of MHC class I molecules (including HLA class I) present on other cells and inhibit NK cell-mediated lysis.

### KIR-CARs

KIRs, referred to as killer cell immunoglobulin-like receptors, have been characterized in humans and non-human primates, and are polymorphic type 1 transmembrane molecules present on certain subsets of lymphocytes, including NK cells and some T cells. KIRs interact with determinants in the alpha 1 and 2 domains of the MHC class I molecules and, as described elsewhere herein, distinct KIRs are either stimulatory or inhibitory for NK cells.

KIRs are a family of cell surface proteins found on NK cells. They regulate the killing function of these cells by interacting with MHC class I molecules, which are expressed on all cell types. This interaction allows them to detect virally infected cells or tumor cells. Most KIRs are inhibitory, meaning that their recognition of MHC suppresses the cytotoxic activity of the NK cell that expresses them. Only a limited number of KIRs have the ability to activate cells.

The KIR gene family has at least 15 gene loci (KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3 and two pseudogenes, KIR2DP1 and KIR3DP1) encoded within a 100-200 Kb region of the Leukocyte Receptor Complex (LRC) located on chromosome 19 (19q13.4). The LRC constitutes a large, 1 Mb, and dense cluster of rapidly evolving immune genes which contains genes encoding other cell surface molecules with distinctive Ig-like extra-cellular domains. In addition, the extended LRC contains genes encoding the transmembrane adaptor molecules DAP 10 and DAP12. KIR genes vary in length from 4 to 16 Kb (full genomic sequence) and can contain four to nine exons. KIR genes are classified as belonging to one of three groups according to their structural features: (1) Type I KIR2D genes, which encode two extracellular domain proteins with a D1 and D2 conformation; (2) The structurally divergent Type II KIR2D genes which encode two extra-cellular domain proteins with a DO and D2 conformation; and finally (3) KIR3D genes encoding proteins with three extra-cellular Ig-like domains (DO, D1 and D2). Type I KIR2D genes, which include the pseudogene KIR2DP1 as well as KIR2DL1-3 and KIR2DS1-5 genes, possess eight exons as well as a pseudoexon 3 sequence. This pseudoexon is inactivated in Type I KIR2D. In some cases, this is due to a nucleotide substitution located on the intron 2-exon 3 splice-site where its nucleotide sequence exhibits a high-degree of identity to KIR3D exon 3 sequences and possesses a characteristic three base pair deletion. In other cases, a premature stop codon initiates differential splicing of exon 3. Within the Type I KIR2D group of genes, KIR2DL1 and KIR2DL2 share a common deletion in exon 7 distinguishing them from all other KIR in this exon, which results in a shorter exon 7 sequence. Similarly, within Type I KIR2D, KIR2DL1-3 differ from KIR2DS1-5 only in the length of their cytoplasmic tail encoding region in exon 9. The KIR2DP1 pseudogene structure differs from that of KIR2DL1-3 in that the former has a shorter exon 4 sequence, due to a single base pair deletion.

Type II KIR2D genes include KIR2DL4 and KIR2DL5. Unlike KIR3D and Type I KIR2D, Type II KIR2D characteristically have deleted the region corresponding to exon 4 in all other KIR. Additionally, Type IIKIR2D genes differ from Type IKIR2D genes in that the former possess a translated exon 3, while Type I KIR2D genes have an untranslated pseudoexon 3 sequence in its place. Within the Type II KIR2D genes, KIR2DL4 is further differentiated from KIR2DL5 (as well as from other KIR genes) by the length of its exon 1 sequence. In KIR2DL4, exon 1 was found to be six nucleotides longer and to possess an initiation codon different from those present in the other KIR genes. This initiation codon is in better agreement with the ‘Kozak transcription initiation consensus sequence’ than the second potential initiation codon in KIR2DL4 that corresponds to the initiation codon present in other KIR genes. KIR3D genes possess nine exons and include the structurally related KIR3DL1, KIR3DS1, KIR3DL2 and KIR3DL3 genes. KIR3DL2 nucleotide sequences are the longest of all KIR genes and span 16,256 bp in full genomic sequences and 1,368 bp in cDNA. Within the KIR3D group, the four KIR genes differ in the length of the region encoding the cytoplasmic tail in exon 9. The length of the cytoplasmic tail of KIR proteins can vary from 14 amino acid residues long (in some KIR3DS1 alleles) to 108 amino acid residues long (in KIR2DL4 proteins). Additionally, KIR3DS1 differs from KIR3DL1 or KIR3DL2 in that the former has a shorter exon 8 sequence. KIR3DL3 differs from other KIR sequences in that it completely lacks exon 6. The most extreme KIR gene structure difference observed was that of KIR3DP1. This gene fragment completely lacks exons 6 through 9, and occasionally also exon 2. The remaining portions of the gene which are present (exon 1, 3, 4 and 5) share a high level of sequence identity to other KIR3D sequences, in particular to KIR3DL3 sequences.

KIR proteins possess Ig-like domains on their extracellular regions, which in some KIR proteins are involved in HLA class I ligand binding. They also possess transmembrane and cytoplasmic regions which are functionally relevant as they define the type of signal which is transduced to the NK cell. KIR proteins can have two or three Ig-like domains (hence KIR2D or KIR3D) as well as short or long cytoplasmic tails (represented as KIR2DS or KIR2DL). Two domain KIR proteins are subdivided into two groups depending on the origin of the membrane distal Ig-like domains present. Type I KIR2D proteins (KIR2DL1, KIR2DL2, KIR2DL3, KIR2DS1, KIR2DS2, KIR2DS3, KIR2DS4 and KIR2DS5) possess a membrane-distal Ig-like domain similar in origin to the KIR3D D1 Ig-like domain but lack a DO domain. This D1 Ig-like domain is encoded mainly by the fourth exon of the corresponding KIR genes. The Type II KIR2D proteins, KIR2DL4 and KIR2DL5, possess a membrane-distal Ig-like domain of similar sequence to the DO domain present in KIR3D proteins, however, Type II KIR2D lack a D1 domain. Long cytoplasmic tails usually contain two Immune Tyrosine-based Inhibitory Motifs (ITIM) which transduce inhibitory signals to the NK cell. Short cytoplasmic tails possess a positively charged amino acid residue in their transmembrane region which allows them to associate with a DAP 12 signaling molecule capable of generating an activation signal Exceptions to this is KIR2DL4, which contains only one N-terminus ITIM. In addition, KIR2DL4 also possesses a charged residue (arginine) in its transmembrane domain, a feature which allows this receptor to elicit both inhibitory and activating signals. KIR control the response of human NK cells by delivering inhibitory or activating signals upon recognition of MHC class I ligands on the surface of potential target cells.

KIR proteins vary in length from 306 to 456 amino acid residues. Although the differences in protein length are mostly the consequence of the number of Ig-like domains present, cytoplasmic region length diversity is also an influencing factor. The leader peptide of most KIR proteins is about 21 amino acid residues long. However, the presence of a different initiation codon generates a correspondingly longer leader peptide in KIR2DL4 proteins.

The Ig-like domain present in Type II KIR2D proteins and KIR3D proteins is approximately 96 amino acid residues in length. The D1 domain of Type I KIR2D and of KIR3D proteins is 102 amino acid residues long, while the D2 domain of all KIR proteins is 98 amino acid residues long. The length of the stem region varies from the 24 amino acid residues present in most KIR proteins, to only seven amino acid residues in the divergent KIR3DL3 protein. The transmembrane region is 20 amino acid residues long for most KIR proteins, but one residue shorter on KIR2DL1 and KIR2DL2 proteins as a result of a three base pair deletion in exon 7. Finally, the cytoplasmic region of KIR proteins exhibits greater length variations, ranging from about 23 amino acid residues in some KIR3DS1 alleles to about 96 amino acid residues present in KIR3DL2 proteins. In some embodiments, the expressible nucleic acid sequences disclosed herein are free of an Ig-like linker.

Amino acid sequences for human KIR polypeptides (Homo sapiens) are available in the NCBI database, see e.g., accession number NP_037421.2 (GI: 134268644), NP_703144.2 (GL46488946), NP_001229796.1 (GL338968852), NP_001229796.1 (GL338968852), NP_006728.2 (GI: 134268642), NP_065396.1 (GI: 11968154), NP_001018091.1 (GL66267727), NP_001077008.1 (GI: 134133244), NP_036444.1 (GL6912472), NP_055327.1 (GL7657277), NP_056952.2 (GI:71143139), NP_036446.3 (GI: 116517309), NP_001074239.1 (GI: 124107610), NP_002246.5 (GI: 124107606), NP_001074241.1 (GI: 124107604), NP_036445.1 (GL6912474), all of which are incorporated by reference in their entireties.

The nomenclature for KIRs is based upon the number of extracellular domains (KIR2D and KIR3D having two and three extracellular Ig-domains, respectively) and whether the cytoplasmic tail is long (KIR2DL or KIR3DL) or short (KIR2DS or KIR3DS). The presence or absence of a given KIR is variable from one NK cell to another within the NK population present in a single individual. Among humans, there is also a relatively high level of polymorphism of KIR genes, with certain KIR genes being present in some, but not all individuals. The expression of KIR alleles on NK cells is stochastically regulated, meaning that, in a given individual, a given lymphocyte may express one, two, or more different KIRs, depending on the genotype of the individual. The NK cells of a single individual typically express different combinations of KIRs, providing a repertoire of NK cells with different specificities for MHC class I molecules. In some embodiments, the nucleic acid sequences of the disclosure encode proteins that comprise short cytoplasmic tails having no more than about 30, 25, 20, 15, 10, 9, 8, 7, 6, 5, 4, 3, 2 or about 1 amino acid in the cytoplasmic portion of the molecule. In some embodiments, the nucleic acid sequences of the disclosure encode proteins that are activating KIRs comprising one antigen-binding domain that associates with CD19 and one antigen-binding domain that associates with CD20.

Certain KIR gene products cause stimulation of lymphocyte activity when bound to an appropriate ligand. Some activating KIR embodiments have a short cytoplasmic tail with a charged trans-membrane residue that associates with an adapter molecule having an Immunoreceptor Tyrosine-based Activation Motifs (ITAMs) which transduce stimulatory signals to the NK cell. By contrast, inhibitory KIRs have a long cytoplasmic tail containing Immunoreceptor Tyrosine-based Inhibitory Motif (ITIM), which transduce inhibitory signals to the NK cell upon engagement of their MHC class I ligands. The known inhibitory KIRs include members of the KIR2DL and KIR3DL subfamilies. Inhibitory KIRs having two IgK domains (KIR2DL) recognize HLA-C allotypes: KIR2DL2 (formerly designated p58.2) and the closely related, allelic gene product KIR2DL3 both recognize “group 1” HLA-C allotypes (including HLA-Cw1, -3, -7, and -8), whereas KIR2DL1 (p58.1) recognizes “group 2” HLA-C allotypes (such as HLA-Cw2, -4, -5, and -6). The recognition by KIR2DL1 is dictated by the presence of a Lys residue at position 80 of HLA-C alleles. KIR2DL2 and KIR2DL3 recognition is dictated by the presence of an Asn residue at position 80 in HLA-C. Importantly, the great majority of HLA-C alleles have either an Asn or a Lys residue at position 80. Therefore, KIR2DL1, -2, and -3 collectively recognize essentially all HLA-C allotypes found in humans. One KIR with three IgK domains, KIR3DL1 (p70), recognizes an epitope shared by HLA-Bw4 alleles. Finally, KIR3DL2 (p140), a homodimer of molecules with three IgK domains, recognizes HLA-A3 and -A11.

Some embodiments comprise at least two KIR-CAR comprising an extracellular domain that comprises a target-specific binding element otherwise referred to as an antigen binding domain fused to a KIR or functional variant thereof. In some embodiments, the KIR is an activating KIR that comprises a short cytoplasmic tail (that associates with an adapter molecule having an Immunoreceptor Tyrosine-based Activation Motifs (ITAMs) which transduce stimulatory signals to the NK cell (referred elsewhere herein as actKIR-CAR). In some embodiments, the KIR is an inhibitory KIR that comprises a long cytoplasmic tail containing Immunoreceptor Tyrosine-based Inhibitory Motif (ITIM), which transduce inhibitory signals (referred elsewhere herein as inhKIR-CAR). In some instances, it is desirable to remove the hinge region for the activating KIRs when construction an actKIR-CAR. An activating KIR CAR in which the KIR2DS2 hinge was removed, to generate the KIR2S CAR, exhibited enhanced cytolytic activity compared to an actKIR-CAR comprising a full-length wildtype KIR2DS2.

Nucleic acid sequences encoding desired molecules of some embodiments can be obtained using recombinant methods known in the art, such as, e.g., by screening libraries from cells expressing the gene, by deriving the gene from a vector known to include the same, or by isolating directly from cells and tissues containing the same, using standard techniques. Alternatively, the gene of interest can be produced synthetically, rather than cloned.

Some embodiments comprise retroviral or lentiviral vector constructs expressing at least two KIR-CAR that can be directly transduced into a cell. Some embodiments comprise an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the gene to be expressed, and a polyAtail, typically 50-2000 bases in length. RNA so produced can efficiently transfect different kinds of cells. In some embodiments, the mRNA encodes at least two KIR-CAR.

Some embodiments herein comprise a chimeric antigen receptor (CAR) molecule comprising an antigen binding moiety and a killer cell immunoglobulin-like receptor (KIR-CAR). In some embodiments, the KIR-CAR is expressed on the surface of a T cell. Some embodiments herein comprise at least two CAR molecules, each comprising an antigen binding moiety and a killer cell immunoglobulin-like receptor (KIR-CAR). In some embodiments, at least two KIR-CAR are expressed on the surface of a T cell.

### Compositions

### Nucleic Acids

The disclosure relates to compositions comprising a first nucleic acid sequence encoding a first fusion protein. In some embodiments, the first fusion protein comprises an antigen-binding domain. In some embodiments, the antigen-binding domain of the first fusion protein comprises an anti-CD19 amino acid sequence. In some embodiments, the first fusion protein comprises an extracellular domain comprising at least a portion of a KIR polypeptide. In some embodiments, the first fusion protein comprises a transmembrane domain comprising at least a portion of a KIR polypeptide. In some embodiments, the first fusion protein further comprises a cytoplasmic domain comprising at least a portion of a KIR polypeptide. In some embodiments, the composition further comprises a second nucleic acid sequence encoding a second fusion protein. In some embodiments, the second fusion protein comprises an antigen-binding domain. In some embodiments, the antigen binding domain of the second fusion protein comprises an anti-CD20 amino acid sequence. In some embodiments, the second fusion protein further comprises an extracellular domain comprising at least a portion of a KIR polypeptide. In some embodiments, the second fusion protein further comprises a transmembrane domain comprising at least a portion of a KIR polypeptide. In some embodiments, the second fusion protein further comprises a cytoplasmic domain comprising at least a portion of a KIR polypeptide. In some embodiments, the composition comprises a third nucleic acid sequence encoding a DAP12 polypeptide. In some embodiments, one or more of the nucleic acid sequence(s) herein is a DNA sequence. In some embodiments, one or more of the nucleic acid sequence(s) herein is an RNA sequence. In some embodiments, the first and second nucleic acid sequences are positioned on a single nucleic acid molecule, such as a plasmid.

In some embodiments, the first and second nucleic acid sequences are free of a nucleic acid sequence encoding one or more of a 41BB polypeptide, an anti-CD22 binding sequence, and a CD28 domain.

### Anti-CD19 Binding Domains

The anti-CD19 binding domain can be any molecule that binds to CD19 with sufficient affinity and specificity, and is often an antibody or an antibody derivative, such as an scFv, single domain antibody (sdAb), Fab′ fragment, (Fab′)2 fragment, nanobody, diabody, or the like. As used herein, “sufficient affinity and specificity” can refer to the property that, under designated conditions, binding domain binds preferentially to its particular target protein (i.e., CD19) and does not bind in a significant amount to other proteins in a sample or subject. In certain embodiments, the anti-CD19 binding domain will have a sufficiently high binding affinity for CD19, for example, the antibody may bind CD19 with a Kd value of between 1E-07 to 1E-10 M. Antibody affinities may be determined, e.g., by a surface plasmon resonance based assay (such as the BIAcore® assay as described in PCT Application Publication No. WO2005/012359); enzyme-linked immunoabsorbent assay (ELISA); and competition assays (e.g. radioimmunoassays (RIAs)). Alternatively, the anti-CD19 binding domain can be a receptor or a receptor fragment that binds specifically to CD19. The anti-CD19 binding domain can be attached to the rest of the receptor directly (covalently) or indirectly (for example, through the noncovalent binding of two or more binding partners). Antibody derivatives are molecules that resemble antibodies in their mechanism of ligand binding, and include, for example, nanobodies, duobodies, diabodies, triabodies, minibodies, F(ab′)2 fragments, Fab fragments, single chain variable fragments (scFv), single domain antibodies (sdAb), and functional fragments thereof. See for example, D. L. Porter et al., N Engl J Med (2011) 365(8):725-33 (scFv); E. L. Smith et al, Mol Ther (2018) 26(6): 1447-56 (scFv); S. R. Banihashemi et al., Iran J Basic Med Sci (2018) 21(5):455-64 (CD19 nanobody); F. Rahbarizadeh et al Adv Drug Deliv Rev (2019) 141:41-46 (sdAb); S. M. Kipriyanov et al., Int J Cancer (1998) 77(5):763-72 (diabody); F. Le Gall et al., FEBS Lett (1999) 453(1-2): 164-68 (triabody); M. A. Ghetie et al., Blood (1994) 83(5): 1329-36 (F(ab′)2); and M. A. Ghetie et al., Clin Cancer Res (1999) 5(12):3920-27 (F(ab′)2 and Fab′). Antibody derivatives can also be prepared from therapeutic antibodies, for example without limitation, by preparing a nanobody, duobody, diabody, triabody, minibody, F(ab′)2 fragment, Fab fragment, single chain variable fragment (scFv), or single domain antibody (sdAb) based on a therapeutic antibody. Antibody derivatives can also be designed using phage display techniques (see, e.g., E. Romao et al., Curr Pharm Des (2016) 22(43):6500-18).

In some embodiments, the anti-CD19 binding domain is an scFv.

In some embodiments the anti-CD19 amino acid sequence is encoded by a first nucleic acid sequence comprising the nucleic acid sequence of one of SEQ ID NOS: 1-43 from Table 1A. In some embodiments, the anti-CD19 amino acid sequence encoded by a first nucleic acid sequence comprises a functional variant of the nucleic acid sequence of one SEQ ID NOS: 1-43 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 1-43.

In some embodiments, anti-CD19 amino acid sequence encoded by the first nucleic acid sequence comprises a nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, to the amino acid sequence of SEQ ID NO: 18. In some embodiments, anti-CD19 amino acid sequence encoded by the first nucleic acid sequence comprises the nucleic acid sequence of SEQ ID NO: 18.

In some embodiments, the anti-CD19 amino acid sequence encoded by the first nucleic acid sequence comprises the amino acid sequence of one of SEQ ID NOS: 44-86 from Table 1A. In some embodiments the anti-CD19 amino acid sequence encoded by the first nucleic acid sequence comprises the amino acid sequence of a functional variant of one of SEQ ID NOS: 44-86 having at least about 70%, about 75%, about 80%, about 85%, about 90%, about 91%, about 92%, about 93%, about 94%, about 95%, about 96%, about 97%, about 98%, about 99% or more sequence identity to one SEQ ID NOS: 44-86 from Table 1A.

In some embodiments, anti-CD19 amino acid sequence encoded by the first nucleic acid sequence comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, to the amino acid sequence of SEQ ID NO: 61. In some embodiments, anti-CD19 amino acid sequence encoded by the first nucleic acid sequence comprises the amino acid sequence of SEQ ID NO: 61.

TABLE 1A
Exemplary anti-CD19 scFv nucleotide and amino acid sequences
(VL-linker-VH)

| SEQ | | | |
|---|---|---|---|
| ID | | | |
| NO: | Name | Type | Sequence |
| 1 | scFv 1 | DNA | CAGGCTGTGCTGAATCAGCCGGCCTCTGTGTCTGCAGCCCTGGGGCAG |
| | | | AGGGTCACCATCTCCTGTAACACCAACATCGGCAGTCCTTATGATGTAC |
| | | | AATGGTACCAGCAGCTCCCAGGAAAGTCCCCTAAAACTATCATTTATGG |
| | | | TAATAGCAATCGACCCTCGGGGGTCCCGGTTCGATTCTCTGGCTCCAAG |
| | | | TCAGGCAGCACAGCCACCCTGACCATCGCTGGGATCCAGGCTGAGGAT |
| | | | GAGGCTGATTATTACTGCCAGTCCTATGATGACAGCCTCGATGGTCGTG |
| | | | TTGTGTTCGGCGGAGGGACCCAACTGACCGTCCTCGGTGGTGGTTCCT |
| | | | CTAGATCTTCCTCCTCTGGTGGCGGTGGCTCGGGCGGTGGTGGGGAGG |
| | | | TGCAGCTGGTGGAGTCTGGGGGAGACCTGGTGAAGCCTGGGGGGTCC |
| | | | TTGAGACTCTCCTGTGTGGCCTCTGGATTCACCTTCAGTAACCACGAA |
| | | | ATGTACTGGGTCCGCCAGGCTCCAGGGAAAGGGCTGGAGTGGGTCGC |
| | | | AAGGATTTATGATAGTGGAAGTAGAACAAGGTATGCAGACGCTGTGAA |
| | | | GGGCCGATTCACCATCTCCAGAGACAACGCCAAGAACACGCTGTATCT |
| | | | TCAGATGAACAGCCTGACAGCCGAGGACACGGCCGTTTATTTCTGTGC |
| | | | GGGGGGGAATTCTCGTGGTTGGACCGAGTTTGGTATGGACTACTGGGG |
| | | | CCCGGGAACCCTGGTCACCGTCTCCTCA |
| 44 | scFv 1 | AA | QAVLNQPASVSAALGQRVTISCNTNIGSPYDVQWYQQLPGKSPKTIIYGNS |
| | | | NRPSGVPVRFSGSKSGSTATLTIAGIQAEDEADYYCQSYDDSLDGRVVFG |
| | | | GGTQLTVLGGGSSRSSSSGGGGSGGGGEVQLVESGGDLVKPGGSLRLSCV |
| | | | ASGFTFSNHEMYWVRQAPGKGLEWVARIYDSGSRTRYADAVKGRFTISR |
| | | | DNAKNTLYLQMNSLTAEDTAVYFCAGGNSRGWTEFGMDYWGPGTLVTV |
| | | | SS |
| 2 | scFv 2 | DNA | TCCTATGTGCTGACTCAGTCACCCTCACTGTCTGGGTCCCTGGGCCAG |
| | | | AAGGTCACCATCTCCTGCACTGGAAGTCGTTCCAATCTCGGTGGAAAT |
| | | | TATGTGGGCTGGTACCAACAAGTCCCAGGAATGGCCCCTAGAAATGTC |
| | | | ATATATGCTAATGATTTTCGACCTTCACGGATTCCCGCTCGATTCTCCGC |
| | | | CTCCAAGTCAGGCACTTCAGCCACCCTGACCATCTCTGGGCTCCAGGC |
| | | | TGAGGACGAGGCTGACTATTTTTGTTCATCATGGGATGATGATCTCGGG |
| | | | AGTCACGTGTTCGGCTCAGGGACCCAGCTGACCATCCTCGGTGGTGGT |
| | | | TCCTCTAGATCTTCCTCCTCTGGTGGCGGTGGCTCGGGCGGTGGTGGG |
| | | | GAGGAGCAACTGGTGGAGTTTGGGGGAGACTTGGTGAAGCCTGGGGG |
| | | | GTCCCTGAGACTCTCCTGTGTAGCCTCTGGATTCAGTTTCAGTAGCTAC |
| | | | GACATGAGCTGGGTCCGCCAGGCTCCTGGGAAGGGGCTGCAATGGGT |
| | | | CGCATCTACTAACTTTGATGGAAGTCGCACATACTACACTGACGCTGTG |
| | | | GAGGGCCGATTCACCATCTCCAGAGACAACGCCAGGAACACAGTGTAT |
| | | | CTGCAGATGAACAGCCTGAGAGCCGAGGACACGGCTGTGTATTACTGT |
| | | | GCGAGGGGGATGGGTAGTTGGGGCGCTTCGACCTTTGGGTACTGGGGC |
| | | | CAGGGAACCCTGGTCACCGTCTCCTCA |
| 45 | scFv 2 | AA | SYVLTQSPSLSGSLGQKVTISCTGSRSNLGGNYVGWYQQVPGMAPRNVI |
| | | | YANDFRPSRIPARFSASKSGTSATLTISGLQAEDEADYFCSSWDDDLGSHV |
| | | | FGSGTQLTILGGGSSRSSSSGGGGSGGGGEEQLVEFGGDLVKPGGSLRLSC |
| | | | VASGFSFSSYDMSWVRQAPGKGLQWVASTNEDGSRTYYTDAVEGRFTIS |
| | | | RDNARNTVYLQMNSLRAEDTAVYYCARGMGSWGASTFGYWGQGTLVT |
| | | | VSS |
| 3 | scFv 3 | DNA | CAGTCTGTGCTGAATCAGCCACCTTCTGTGTCTGGAGCCCTGGGGCAG |
| | | | ACCGTCACCATCTCCTGCTCTGGAAGTCACACCAACATCGGCAGTGGA |
| | | | TTTGATGTACAGTGGTACCAACAGCTCCCAGGAAAGTCTCCTCAAACT |
| | | | CTCGTCTATGCTAATATCAATCGACCCTCGGGGGTCCCGGCTCGATTCT |
| | | | CTGGCTCCAAGTCAGGCAGCACAGCCACCCTGACCATCACTGGGGTCC |
| | | | AGGCTGAAGATGAGGCTGATTATTACTGCCAGTCGTATGATGACAACTT |
| | | | CGATGGGCACGTGTTCGGCTCAGGAACCCAACTGACCGTCCTCGGCG |
| | | | GTGGTTCCTCTAGATCTTCCTCCTCTGGTGGCGGTGGCTCGGGCGGTG |
| | | | GTGGGGAGGTCCAGCTGGTGCAGTCTGGGGGAGACCTGGTGAAACCT |
| | | | GGGGGATCCCTGAGACTGTCCTGTGTGGCCTCTGGATTCACCTTCAGT |
| | | | AACTACACCATGAGCTGGGTCCGCCAGGCTCCAGGGCAGGGGCTGCA |
| | | | GTGGGTCGCATACATTAACTATGATGGGAGTATCACATACTACGCAGAG |
| | | | GCTGTGAAGGGCCGGTTCACCGTCTCCAGAGACAACGCCAAGAACAC |
| | | | GCTGTATCTGCAGATGAACAGTCTGAGAGCCGAGGACACGGCCGTGTA |
| | | | TTTCTGTGTGAAGGAAACTTTCATGATACCAACTGAGTGGGGCCAGGG |
| | | | AACCCTTATCACCGTCTCCTCA |
| 46 | scFv 3 | AA | QSVLNQPPSVSGALGQTVTISCSGSHTNIGSGFDVQWYQQLPGKSPQTLV |
| | | | YANINRPSGVPARFSGSKSGSTATLTITGVQAEDEADYYCQSYDDNFDGH |
| | | | VFGSGTQLTVLGGGSSRSSSSGGGGSGGGGEVQLVQSGGDLVKPGGSLRL |
| | | | SCVASGFTFSNYTMSWVRQAPGQGLQWVAYINYDGSITYYAEAVKGRFT |
| | | | VSRDNAKNTLYLQMNSLRAEDTAVYFCVKETFMIPTEWGQGTLITVSS |
| 4 | scFv 4 | DNA | CAGTCTGTGCTGAATCAGCCGCCTTCTGTGTCTGGAGCCCTGGGGCAG |
| | | | ACCGTCACCATCTCCTGTTCTGGAAGTCACACCAACATCGGCAGTGGA |
| | | | TTTGATGTACAGTGGTACCAACAGCTCCCAGGAAAGTCCCCTCAAACT |
| | | | ATCATCTATGCTAATATCAATCGACCCTCGGGGGTCCCGGCTCGATTCTC |
| | | | TGGCTCCAAGTCAGGCAGCACAGCCACCCTGACCATCACTGGGGTCC |
| | | | AGGCTGAAGATGAGGCTGATTATTACTGCCAGTCGTATGATGACAATTT |
| | | | CGATTACGTGTTCGGCTCAGGGACCCAACTGACCGTCCTTGGTGGTGG |
| | | | TTCCTCTAGATCTTCCTCCTCTGGTGGCGGTGGCTCGGGCGGTGGTGG |
| | | | GGAGGAGCAACTGGTGGAGTTTGGGGGAGACCTGGTGAAGCCTGGAG |
| | | | GGTCCTTGAGACTGTCCTGTGTGGTCTCTGGATTCGATTTCAGTCGCTA |
| | | | CGGCATGTCCTGGGTCCGTCAGTCTCCAGGGAAGGGTCTCCAGTGGGT |
| | | | CGCAGATATCAGTTACAGGGGAACTACATTTTACGCAGACGATGTGAA |
| | | | GGGCCGATTTACCGTCTCCAGAGACAGTGCCAAGAACACACTCTACCT |
| | | | CGAAATGACCAGCCTGACAGTCGAGGACACGGCCGTATATTATTGTGC |
| | | | GAATAGTGCCAACTGGTATGTCTCCCCTTTTGACCACTGGGGCCAGGG |
| | | | AACCCTGGTCACCGTCTCCTCA |
| 47 | scFv 4 | AA | QSVLNQPPSVSGALGQTVTISCSGSHTNIGSGFDVQWYQQLPGKSPQTIIY |
| | | | ANINRPSGVPARFSGSKSGSTATLTITGVQAEDEADYYCQSYDDNFDYVF |
| | | | GSGTQLTVLGGGSSRSSSSGGGGSGGGGEEQLVEFGGDLVKPGGSLRLSC |
| | | | VVSGFDFSRYGMSWVRQSPGKGLQWVADISYRGTTFYADDVKGRFTVS |
| | | | RDSAKNTLYLEMTSLTVEDTAVYYCANSANWYVSPFDHWGQGTLVTVSS |
| 5 | scFv 5 | DNA | CAGCCTGTGCTGACCCAGCCACCCTCAGTGTCGGGGTCCCTTGGCCAG |
| | | | AGGGTCACCATCTCCTGTTCTGGAAGCACGAACACCATCGGTGTTGTT |
| | | | GGTGCGAACTGGTACCAACAGCTCCCAGGAAAGTCCCCTAAACTCCTC |
| | | | GTGTACAGTGATGGGGTTCGACCGTCAGGGGTCCCTGACCGGTTTTCC |
| | | | GGCTCCAGGTCTGCCAACTCAGACACCCTGACCATCACTGGGCTTCAG |
| | | | GCTGAGGACGAGGCTGATTATTACTGCCAGTCCTTTGATTCCACACATA |
| | | | ATGCCATTGTATTCGGCGGAGGCACCCATCTGACCGTCCTCGGTGGTGG |
| | | | TTCCTCTAGATCTTCCTCCTCTGGTGGCGGTGGCTCGGGCGGTGGTGG |
| | | | GGAGGTGCAGCTGGTGGAGACTGGGGGAGACCTGGTGAAGCCTGCGG |
| | | | GGTCCCTGAGACTGTCGTGTGCGGCCTCTGGATTCCCCTTCAGTGGCT |
| | | | ACAGTATGACCTGGGTCCGCCAGGCCCCTGAAAAGGGTCTGCAATTGG |
| | | | TCGCAGGTATTAACAGCGATGGAAGTCACACATACTACACAGACGATG |
| | | | TGAAGGGCCGATTCACCATCTCCAGAGACAACACCAAATACATACTGT |
| | | | ATCTGCAAATGAACAGTCTGAGAGCCGAGGACACGGCCATGTATTATT |
| | | | GTGGCTCTCATAGTTGGGGCAATTTTCACTACTGGGGCCAGGGAACCC |
| | | | TGGTCACCGTCTCCTCA |
| 48 | scFv 5 | AA | QPVLTQPPSVSGSLGQRVTISCSGSTNTIGVVGANWYQQLPGKSPKLLVYS |
| | | | DGVRPSGVPDRFSGSRSANSDTLTITGLQAEDEADYYCQSFDSTHNAIVF |
| | | | GGGTHLTVLGGGSSRSSSSGGGGSGGGGEVQLVETGGDLVKPAGSLRLSC |
| | | | AASGFPFSGYSMTWVRQAPEKGLQLVAGINSDGSHTYYTDDVKGRFTISR |
| | | | DNTKYILYLQMNSLRAEDTAMYYCGSHSWGNFHYWGQGTLVTVSS |
| 6 | scFv 6 | DNA | CAGCCTGTGCTCACTCAGCCGCCCTCAGTGTCTGGGTCCCTGGGCCAG |
| | | | AGGGTCACCATCTCCTGCACCGGAACTAATACCAACATCGGCAGTGGT |
| | | | TATAGTGTACAATGGTACCAGCAGCTCCCAGGAGAGTCCCCAAAACCT |
| | | | ATCATTTATGGTAGTAGTAATCGACCCTCGGGGGTCCCGGCTCGATTCT |
| | | | CTGGCTCCAAGTCAGGCAGCACAGGCACCCTGACCATCACTGGGATCC |
| | | | AGGCTGAGGATGAGGCTGATTACCACTGCCAGTCCTATGATGACAGCC |
| | | | TCGATGGTCATGCTGTATTCGGCGGAGGGACCCAGCTGACCATCCTCG |
| | | | GTGGTGGTTCCTCTAGATCTTCCTCCTCTGGTGGCGGTGGCTCGGGCG |
| | | | GTGGTGGGGAGGTCCAGCTGGTGCAGTCTGGGGGAGACTTGGTGAAG |
| | | | CCTGGGGGGTCCTTGAGACTGTCCTGTGTGGCCTCTGGATTCACCTTC |
| | | | AGTGACAATTACATGAGCTGGATCCGTCAGGTTCCAGGGAAGGGCCTG |
| | | | CAGTGGGTCTCACGAATTAGCACTGATGGAAGAGGCACAAGCTACGC |
| | | | AGACGCTGTGAAGGGCCGATTCACCATCTCCAGAGACAATGCCAAGA |
| | | | ACACGCTGTATCTGCAGATGAACAGCCTGAGAGACGAGGACACGGCC |
| | | | GTGTATTACTGTGCAAGGCGGTCTGGTATTTGGGGGTTTGACTACTGGG |
| | | | GCCAGGGCGCCCTGGTCACCGTCTCCTCA |
| 49 | scFv 6 | AA | QPVLTQPPSVSGSLGQRVTISCTGTNTNIGSGYSVQWYQQLPGESPKPIIY |
| | | | GSSNRPSGVPARFSGSKSGSTGTLTITGIQAEDEADYHCQSYDDSLDGHAV |
| | | | FGGGTQLTILGGGSSRSSSSGGGGSGGGGEVQLVQSGGDLVKPGGSLRLS |
| | | | CVASGFTFSDNYMSWIRQVPGKGLQWVSRISTDGRGTSYADAVKGRFTIS |
| | | | RDNAKNTLYLQMNSLRDEDTAVYYCARRSGIWGFDYWGQGALVTVSS |
| 7 | scFv 7 | DNA | TCCTATGAGCTGACTCAGCCACCATCTATGTCTGCAGCCCTGGGGCAG |
| | | | AGGGTCACCATCTCCTGCACTGGAAGTAACACCAACATCGGCAGTGGT |
| | | | TATGATGTACAATGGTACCAACAGGTCCCAGGAAAGTCCCCTACACCT |
| | | | CTGATTTATGGTAATACCAATCGACCCTCGGGGGTCCCGGTGCGATTCT |
| | | | CTGGCTCCAAGTCAGGCAACACAGCCACCCTGACCATCACTGGGATCC |
| | | | AGGCTGAGGATGAGGCTGATTATTACTGCCAGTCCTATGATGACAATTT |
| | | | CGATGGTCATCACGTGTTCGGCTCAGGGACCCAGCTGACCGTCCTCGG |
| | | | TGGTGGTTCCTCTAGATCTTCCTCCTCTGGTGGCGGTGGCTCGGGCGGT |
| | | | GGTGGGGAGGTACAGCTGGTGGAATCTGGGGGAGACCTGGTGAAGCC |
| | | | TGCGGGGTCCCTGAGACTGTCCTGTGTGGCCTCTGGATTCACCTTCAG |
| | | | TAACTATGGCATGAATTGGGTCCGCCAGGCTCCCGGGAAGGGGCTGCA |
| | | | GTGGGTCGCAGGTATTAACAGTGGTGGAAGTACCACAACCTACGCAGA |
| | | | CGTTGTGAAGGGTCGATTCACCATCTCCAGAGACAACGCCAAGAACA |
| | | | CACTGTATCTGCAGATGAACAGCCTGGGAGCCGAGGACACGGCCGTGT |
| | | | ATTACTGTGCGAGGGTGGATTATAGTAGCAGCTGGTTGGTTGACTACTG |
| | | | GGGCCAGGGAACCCTGGTCACCGTCTCCTCA |
| 50 | scFv 7 | AA | SYELTQPPSMSAALGQRVTISCTGSNTNIGSGYDVQWYQQVPGKSPTPLI |
| | | | YGNTNRPSGVPVRFSGSKSGNTATLTITGIQAEDEADYYCQSYDDNFDGH |
| | | | HVFGSGTQLTVLGGGSSRSSSSGGGGSGGGGEVQLVESGGDLVKPAGSLR |
| | | | LSCVASGFTFSNYGMNWVRQAPGKGLQWVAGINSGGSTTTYADVVKGR |
| | | | FTISRDNAKNTLYLQMNSLGAEDTAVYYCARVDYSSSWLVDYWGQGTLV |
| | | | TVSS |
| 8 | scFv 8 | DNA | CAGTCTGTGTTGACTCAACCGGCCTCAGTGTCTGGGTCCCTGGGCCAG |
| | | | AAGGTCACCATCTCCTGCACTGGAAGCAGGTCCAACATCGGTGCAAAT |
| | | | TATGTGGGCTGGTATCAACAGGTCCCAGGAATAGGCCCTAGAACCGTC |
| | | | ATTTTTGGTTATAATTATCGACCTTCAGGGGTCCCCGATCGATTCTCTAG |
| | | | CTCCAAGTCAGGCAATTCAGCCACCCTGACCATCTCTGGGCTCCAGGC |
| | | | TGAGGACGAGGCTGAATATTATTGTTCATCCTGGGATGATAGTCTCAGA |
| | | | GGTACCGTGTTCGGCTCAGGGACCCAACTGACCGTCCTTGGCGGTGGT |
| | | | TCCTCTAGATCTTCCTCCTCTGGTGACGGTGGCTCGGGCGGTGGTGGG |
| | | | GAGGTACAGCTGGTGGAATCTGGGGGAGACCTGGTGAAGCCTGGGGA |
| | | | ATCCCTGAGACTGTCCTGTGTGGCCTCTGGATTCACCTTCAGTAGCTAT |
| | | | GGCATGAGCTGGGTCCGACAGTCTCCAGGGAAGGGGTTGCAGTGGGT |
| | | | CGCAGATATTAGTTATGGAGGAGGTACATACTACGCAGACGCTGTGAA |
| | | | GGGCCGATTCACCATCTCCAGAGACAACGCCAGGAACACAGTATATCT |
| | | | GCAGATGAACAGCCTGAGAGCCGAGGACACGGCCGTGTATTACTGTGC |
| | | | GTCCCAATGGGGTGATTGGGGCCACTTTGAGTACTGGGGCCAGGGCAC |
| | | | CCTGGTCACCGTCTCCTCA |
| 51 | scFv 8 | AA | QSVLTQPASVSGSLGQKVTISCTGSRSNIGANYVGWYQQVPGIGPRTVIFG |
| | | | YNYRPSGVPDRFSSSKSGNSATLTISGLQAEDEAEYYCSSWDDSLRGTVF |
| | | | GSGTQLTVLGGGSSRSSSSGDGGSGGGGEVQLVESGGDLVKPGESLRLSC |
| | | | VASGFTFSSYGMSWVRQSPGKGLQWVADISYGGGTYYADAVKGRFTISR |
| | | | DNARNTVYLQMNSLRAEDTAVYYCASQWGDWGHFEYWGQGTLVTVSS |
| 9 | scFv 9 | DNA | CAGCCTGTGCTGACCCAGCCACCCTCTGTGTCTGCAGCCCTGGGACAG |
| | | | AGGGTCACCCTCTCCTGCACTGGAAGTAAGACCAACATCGGCAGTGGT |
| | | | TATGATGTACAATGGTACCAGCAGTTCCCAGGAAAGTCCCCTAAAACC |
| | | | ATCATCTATGGTAATAGCAATCGATTTTCGGGGGTCCCGGCTCGATTCTC |
| | | | TGGCTCCAAGTCAGGCAGCATCGCCACCCTGACCATCACTGGGATCCA |
| | | | GGCTGAGGATGAGGCTGATTATTACTGCCAGTCCTTTGATGACAATGTC |
| | | | GATGGTTACGTGTTCGGCTCAGGGACCCAGCTGACCATCCTCGGTGGT |
| | | | GGTTCCTCTAGATCTTCCTCCTCTGGTGGCGGTGGCTCGGGCGGTGGT |
| | | | GGGGAGGTACAGCTGGTGGAGTCTGGGGGAGACCTGGTGAAGCCTGG |
| | | | GGGGTCCCTGAGACTCTCCTGTGTGGCCTCTGGATTCACCTTCAGTAG |
| | | | CGACAGCATACATTGGGTCCGTCAGGCTCCAGGAAAGGGGCTGCAGT |
| | | | GGGTCGCATACATTAACAGTGGTGGAAGTACCACATACTACGCAGACG |
| | | | CTGTGAAGGGCCGATTCACCATCTCCAGAGACAACGCCAGGAACACA |
| | | | GTGTATCTGAAGATGAACAGCCTGAGAGCCGAGGACACGGCTGTGTAT |
| | | | TACTGTGCGAAAGATTCAGGCTTTGGCAGCTGGTACCTTGAATCCTGG |
| | | | GGCCAGGGAACCCTGGTCACCGTCTCCCCA |
| 52 | scFv 9 | AA | QPVLTQPPSVSAALGQRVTLSCTGSKTNIGSGYDVQWYQQFPGKSPKTIIY |
| | | | GNSNRFSGVPARFSGSKSGSIATLTITGIQAEDEADYYCQSFDDNVDGYVF |
| | | | GSGTQLTILGGGSSRSSSSGGGGSGGGGEVQLVESGGDLVKPGGSLRLSC |
| | | | VASGFTFSSDSIHWVRQAPGKGLQWVAYINSGGSTTYYADAVKGRFTISR |
| | | | DNARNTVYLKMNSLRAEDTAVYYCAKDSGFGSWYLESWGQGTLVTVSP |
| 10 | scFv 10 | DNA | CAGGCTGTGCTGAATCAGCCGGCCTCTGTGTCTGGAGCCCTGGGGCAG |
| | | | ACCGTCACCATCTCCTGTTCTGGAAGTCACACCAACATCGGCAGTGGA |
| | | | TTTGATGTACAGTGGTACCAACAGCTCCCAGGAAAGTCCCCTCAAACT |
| | | | ATCATCTATGCTAATATCAATCGACCCTCGGGGGTCCCGGCTCGATTCTC |
| | | | TGGCTCCAAGTCAGGCAGCACAGCCACCCTGACCATCACTGGGGTCC |
| | | | AGGCTGAAGATGAGGCTGATTATCACTGCCAGTCCTTTGATGACGACC |
| | | | TCGATGGTCCTGTGTTCGGCGGAGGCACCCATCTGACCGTCCTCGGTG |
| | | | GTGGTTCCTCTAGATCTTCCTCCTCTGGTGGCGGTGGCTCGGGCGGTG |
| | | | GTGGGGAGGTCCAGCTGGTGCAGTCTGGAGGAGACCTGGTGAAGCCT |
| | | | GGGGGGTCCCTGAAAATTTCCTGTGTGGCCTCTGGACTCACCTTCAAT |
| | | | AGAAACTACATGACCTGGATCCGCCAGGCTCCAGGGAAGGGGCTGCA |
| | | | GTGGGTCTCAGAAATTAACCCTGATGGAAGTACCACGAGTTACACAGA |
| | | | CGCTGTGAGGGGCCGATTCACCATCTCCAGAGACAATGCCAAGAACAC |
| | | | GTTGTATCTGCAGATGAATAGTATGAGAGATGAGGACACGGCAGTGTAT |
| | | | TACTGCGCAAGGAGTAGTTGGGGTTCTTTTGACTATTGGGGCCAGGGA |
| | | | ACCCTGGTCACCGTCTCCTCA |
| 53 | scFv 10 | AA | QAVLNQPASVSGALGQTVTISCSGSHTNIGSGFDVQWYQQLPGKSPQTIIY |
| | | | ANINRPSGVPARFSGSKSGSTATLTITGVQAEDEADYHCQSFDDDLDGPVF |
| | | | GGGTHLTVLGGGSSRSSSSGGGGSGGGGEVQLVQSGGDLVKPGGSLKISC |
| | | | VASGLTFNRNYMTWIRQAPGKGLQWVSEINPDGSTTSYTDAVRGRFTISR |
| | | | DNAKNTLYLQMNSMRDEDTAVYYCARSSWGSFDYWGQGTLVTVSS |
| 11 | scFv 11 | DNA | CAGCCTGTGCTGACCCAGCCACCCTCTGTGTCTGGAGCCCTGGGGCAG |
| | | | ACCGTCACCATCTCCTGCTCTGGAAGTCACACCAACATCGGCAGTGGA |
| | | | TTTGATGTACAGTGGTACCAACAGCTCCCAGGAAAGTCCCCTCAAACT |
| | | | CTCGTCTATGCTAATATCAATCGACCCTCGGGGGTCCCGGCTCGATTCT |
| | | | CCGGCTCCAAGTCAGGCAGCACAGCCACCCTGACCATCACTGGGGTC |
| | | | CAGGCTGAAGATGAGGCTGATTATTACTGCCAGTCGTATGATGACAATT |
| | | | TCGATTACGTGTTCGGCTCAGGGACCCAACTGACCGTCCTTGGTGGTG |
| | | | GTTCCTCTAGATCTTCCTCCTCTGGTGGCGGTGGCTCGGGCGGTGGTG |
| | | | GGGAGGTACAGCTGGTGGAATCTGGGGGAGACCTGGTGAAGCCTGGG |
| | | | GGGTCCTTGAGACTGTCCTGTGTGGCCTCTGGATTCACCTTCAGTGAC |
| | | | TATAGCATGAGCTGGGTCCGTCAGTCTCCAGGGAAGGGGCTGCAGTGG |
| | | | GTCGCAGCTATTATGACTAGTGGAAGCACATACTACGCAGACGCTGTG |
| | | | AAGGGCCGATTCACCATCTCCAGAGACAACGCCAAGAATACGCTGTAT |
| | | | CTGCAGATGGACAGCCTGAGAGCCGAGGACACGGCCGTTTATTACTGT |
| | | | GCGCACGACTACTGGTTTGACTTCTGGGGCCAGGGCACCCTGGTCACC |
| | | | GTCTCCTCA |
| 54 | scFv 11 | AA | QPVLTQPPSVSGALGQTVTISCSGSHTNIGSGFDVQWYQQLPGKSPQTLV |
| | | | YANINRPSGVPARFSGSKSGSTATLTITGVQAEDEADYYCQSYDDNFDYV |
| | | | FGSGTQLTVLGGGSSRSSSSGGGGSGGGGEVQLVESGGDLVKPGGSLRLS |
| | | | CVASGFTFSDYSMSWVRQSPGKGLQWVAAIMTSGSTYYADAVKGRFTIS |
| | | | RDNAKNTLYLQMDSLRAEDTAVYYCAHDYWFDFWGQGTLVTVSS |
| 12 | scFv 12 | DNA | TCCTATGAGCTGACTCAGCCACCATCAGCGACTGTGACCCTGAGGCAG |
| | | | ACGGCCCGCCTCACCTGTGGGGGAGAGAACATTGGAAGAAAAAGTGT |
| | | | TCAATGGTACCAACAGAAGCCGGGCCAGTCCCCCCTACTCATTATCTAT |
| | | | GCTGATTCCAGCAGGCCGTCAGGGATCCCTGAGCGATTCTCTGGCGCC |
| | | | AATTCGGGGAACACGGCCAGCCTGACCATCAGCGGGGCCCTGGCCGA |
| | | | GGACGAGGCTGACTATTACTGCCAGATTTGGGACCGCAATATTAATGTG |
| | | | TTCGGCTCCGGGACCCAGCTGACCGTCCTTGGCGGTGGTTCCTCTAGA |
| | | | TCTTCCTCCTCTGGTGGCGGTGGCTCGGGCGGTGGTGGGGAGGAGCA |
| | | | ACTGGTGGAGTTTGGGGGAGACCTGGTGAAGCCTGCGGGGTCCCTGA |
| | | | GACTGTCCTGTGTGGCCTCGGGATTCACCTTCAGTAGCTACGACATGA |
| | | | GCTGGGTCCGCCAGGCTCCTGGGAAGGGGCTGCAGTGGGTCGCGACT |
| | | | ATTAGTTATGATGGAAGTAGTACATACTACACTGACGCTGTGAAGGGCC |
| | | | GATTCACCGTCTCCAGAGACAACGCCAAGAACACGTTGTATCTGCAGA |
| | | | TGAACAGCCTGAGAGCCGAGGACACGGCTCTGTATTACTGTGCAAAG |
| | | | GATTCTGGCAGTTGGTCCCATTTCTATGGTTTGGACACCTGGGGCCATG |
| | | | GCACCTCAGTCTTCGTGTCTTCC |
| 55 | scFv 12 | AA | SYELTQPPSATVTLRQTARLTCGGENIGRKSVQWYQQKPGQSPLLIIYADS |
| | | | SRPSGIPERFSGANSGNTASLTISGALAEDEADYYCQIWDRNINVFGSGTQ |
| | | | LTVLGGGSSRSSSSGGGGSGGGGEEQLVEFGGDLVKPAGSLRLSCVASGF |
| | | | TFSSYDMSWVRQAPGKGLQWVATISYDGSSTYYTDAVKGRFTVSRDNAK |
| | | | NTLYLQMNSLRAEDTALYYCAKDSGSWSHFYGLDTWGHGTSVFVSS |
| 13 | scFv 13 | DNA | CAGCCTGTGCTGACCCAGCCACCCTCTGTGTCTGGAGCCCTGGGGCAG |
| | | | ACCGTCACCATCTCCTGCTCTGGAAGTCACACCAACATCGGCAGTGGA |
| | | | TTTGATGTACAGTGGTACCAACAGCTCCCAGGAAAGTCCCCTCAAACT |
| | | | CTCGTCTATGCTAATATCAATCGACCCTCGGGGGTCCCGGCTCGATTCT |
| | | | CTGGCTCCAAGTCAGGCAGCACAGCCACCCTGACCATCACTGGGGTCC |
| | | | AGGCTGAAGATGAGGCTGATTATTACTGCCAGTCGTATGATGACAATTT |
| | | | CGATTACGTGTTCGGCTCAGGGACCCAGCTGACCGTCCTCGGCGGTGG |
| | | | TTCCTCTAGATCTTCCTCCTCTGGTGACGGTGGCTCGGGCGGTGGTGG |
| | | | GGAGGTACAGCTGGTGGAGTCTGGGGGAGGCCTGATGAAGCCTGGGG |
| | | | GGTCCCTGAAACTCTCCTGTTTGGCCTCTGGTTTCACCTTCAGTCGCTA |
| | | | CTATATGTATTGGGTCCGCCAGGCTCCAGGGAAGGGACTTCAGTGGGT |
| | | | CGCACAAATTAGCAGTGATGGCAGAAGAACATACTACGCAGACGCTGT |
| | | | GCAGGGCCGATTCACCATCTCCAGAGACAATGTCAAGAACACGGTATA |
| | | | TCTGCAGATGAACAGCCTGAGCGACGAGGACACGGCCGTGTATTATTG |
| | | | TGTCCGAGAGGACTACTTCGACTTTGACTACTGGGGCCAGGGCACCCT |
| | | | GGTCACCGTCTCCTCA |
| 56 | scFv 13 | AA | QPVLTQPPSVSGALGQTVTISCSGSHTNIGSGFDVQWYQQLPGKSPQTLV |
| | | | YANINRPSGVPARFSGSKSGSTATLTITGVQAEDEADYYCQSYDDNFDYV |
| | | | FGSGTQLTVLGGGSSRSSSSGDGGSGGGGEVQLVESGGGLMKPGGSLKL |
| | | | SCLASGFTFSRYYMYWVRQAPGKGLQWVAQISSDGRRTYYADAVQGRFT |
| | | | ISRDNVKNTVYLQMNSLSDEDTAVYYCVREDYFDFDYWGQGTLVTVSS |
| 14 | scFv 14 | DNA | CAGTCTGTGCTGAATCAGCCACCTTCTGTGTCTGCGGCCCTGGGGCAG |
| | | | AGGATCACCATCTCCTGCACTGGAAGTCACACCAACATCGGCAGTGGT |
| | | | TATGATGTACAATGGTACCAGCAGCTCCCCGGAAAGTCCCCTAGAACT |
| | | | ATCATTTATGGTAATAGCGATCGGCCCTCTGGGGTCCCGGTTCGATTCTC |
| | | | TGGCTCCAAGTCAGGCAACACAGCCACCCTGACCATCACTGGGATCCA |
| | | | GGCTGAGGATGAGGCTGATTATTATTGCCAGTCCTATGATGACAACCTC |
| | | | AATGATCCTTACGTGTTCGGCTCAGGGACCCAACTGACCGTCCTTGGT |
| | | | GGTGGTTCCTCTAGATCTTCCTCCTCTGGTGGCGGTGGCTCGGGCGGT |
| | | | GGTGGGGAGGTCCAGCTGGTGCAGTCTGGGGGAGACCTGGTGAAGCC |
| | | | TGGGGGGTCCCTGAGACTCTCCTGTGTGGCCTCTGGATTCGCCTTCAG |
| | | | TGGCAAAACAATGCAATGGGTCCGTCAGGCTCCAGGGAAGGGGCTAC |
| | | | AGTGGGTCGCATACATTCAACATGATGGGAGTAGAACATCCTACGCAG |
| | | | ACGCTGTGAAGGGCCGATTCACCATCTCCAGAGACAACGCCAAGAAC |
| | | | ACGCTCTATCTCCAGATGGACAGCCTGAGAGCCGAGGACACGGCCGTG |
| | | | TATTACTGTGCGGATCATCCCTGGTACGGGCCTGAGTATTGGGGCCAGG |
| | | | GAACCCCGGTCACCGTCTCCTCA |
| 57 | scFv 14 | AA | QSVLNQPPSVSAALGQRITISCTGSHTNIGSGYDVQWYQQLPGKSPRTIIY |
| | | | GNSDRPSGVPVRFSGSKSGNTATLTITGIQAEDEADYYCQSYDDNLNDPY |
| | | | VFGSGTQLTVLGGGSSRSSSSGGGGSGGGGEVQLVQSGGDLVKPGGSLRL |
| | | | SCVASGFAFSGKTMQWVRQAPGKGLQWVAYIQHDGSRTSYADAVKGRFT |
| | | | ISRDNAKNTLYLQMDSLRAEDTAVYYCADHPWYGPEYWGQGTPVTVSS |
| 15 | scFv 15 | DNA | TCCTATGAGCTGACTCAGCCACCATCGGTGTCTGCAGCCCTGGGGCAG |
| | | | GGGGTCACCATCTCCTGCATCGGCAGTGACACCAACATCGGCAATCAT |
| | | | TATGATGTACAATGGTACCAGCAGCTCCCAGGAAAGTCCCCTAGAACT |
| | | | ATTATTTATGGTAATAGCGATCGACCCTCGGGGGTCCCGGGTCGATTCT |
| | | | CTGGCTCCAAGTCAGGCAACACAGCCACCCTGACCATCACTGGGATCC |
| | | | AGGCTGAGGATGAGGCTGATTATCACTGCCAGTCCTATGATGACGACCT |
| | | | CGATGGCCATATTGTGTTCGGCGGAGGCACCCATCTGACCGTCCTCGGT |
| | | | GGTGGTTCCTCTAGATCTTCCTCCTCTGGTGGCGGTGGCTCGGGCGGT |
| | | | GGTGGGGAGGTCCAGCTGGTGCAGTCTGGGGGAGACCTGGTGAAGCC |
| | | | TGGGGGTTCCCTGAGACTGTCCTGTGTGGCATCTGGATTCACCTTCAAT |
| | | | AAGTACCACATGAGTTGGGTCCGCCAGGCTCCAGGGAAGGGGCTGCA |
| | | | GTGGGTCGGAAAGATAACCAGTGATGGAAGTGCCACAGGGTATGCAG |
| | | | ACGCTGTAAAGGGCCGATTCACCATCTCCAGAGACAATGCCAAGAACA |
| | | | CGTTCTATCTACAGATGGACAGCCTGAGAGCCGAGGACACGGCTATGT |
| | | | ATTACTGTGCAAGGGATCAGAGTAGTTGGGGAACGGGTAATTTTGACT |
| | | | ACTGGGGCCAGGGAACCCTGGTCACCGTCTCCTCA |
| 58 | scFv 15 | AA | SYELTQPPSVSAALGQGVTISCIGSDTNIGNHYDVQWYQQLPGKSPRTIIY |
| | | | GNSDRPSGVPGRFSGSKSGNTATLTITGIQAEDEADYHCQSYDDDLDGHI |
| | | | VFGGGTHLTVLGGGSSRSSSSGGGGSGGGGEVQLVQSGGDLVKPGGSLR |
| | | | LSCVASGFTFNKYHMSWVRQAPGKGLQWVGKITSDGSATGYADAVKGR |
| | | | FTISRDNAKNTFYLQMDSLRAEDTAMYYCARDQSSWGTGNFDYWGQGT |
| | | | LVTVSS |
| 16 | scFv 16 | DNA | CAGTCTGTGCTGACTCAGCCGACCTCAGTGTCTGGGTCCCTGGGCCAG |
| | | | AGGGTCACCATCTCCTGCACTGGAAGCAGCTCCAATGTTGGTTATGGC |
| | | | AATTATGTGGGCTGGTACCAGCAGCTCCCAGGAACAGGCCCCAGAACC |
| | | | CTCATCTATGGTAGTCATTACCGACCCTCGGGGGTCCCTGATCGATTCT |
| | | | CTGGCTCCAGTTCAGGCAGCTCAGCCACACTGACCATCTCTGGGCTCC |
| | | | AGGCTGAGGATGAGGCTGATTATTACTGCTCATCCTACGACAGCACTCT |
| | | | CAGTGGTTACGTGTTTGGCTCAGGGACCCAGCTGACCATCCTCGGCGG |
| | | | TGGTTCCTCTAGATCTTCCTCCTCTGGTGGCGGTGGCTCGGGCGGTGGT |
| | | | GGGGAGGTACAGCTGGTGGAATCTGGGGGAGACCTGGTGAAGCCTGG |
| | | | GGGGTCCCTGAGACTCTCCTGTGTGGGCTCTGGATTCACCTTGAGTAG |
| | | | TTATTGGATGAGCTGGGTCCGCCAGGCTCCAGGGAAGGGGCTACAGTG |
| | | | GATTGCAGAAATCAGCGGCAGTGGAAATACCATATATTATGGGGACGCT |
| | | | GTGAAGGGCCGATTCACCATCTCCAGAGACAATGCCAAGAACACGCT |
| | | | GTATCTGCAGATGAACAGCCTGAGAGATGAGGACACGGCAGTGTATTA |
| | | | CTGTGCAACGGAATACTACTGGAATATTGACAACTGGGGCCAGGGAAC |
| | | | CCTGGTCACCGTCTCCTCA |
| 59 | scFv 16 | AA | QSVLTQPTSVSGSLGQRVTISCTGSSSNVGYGNYVGWYQQLPGTGPRTLI |
| | | | YGSHYRPSGVPDRFSGSSSGSSATLTISGLQAEDEADYYCSSYDSTLSGYV |
| | | | FGSGTQLTILGGGSSRSSSSGGGGSGGGGEVQLVESGGDLVKPGGSLRLSC |
| | | | VGSGFTLSSYWMSWVRQAPGKGLQWIAEISGSGNTIYYGDAVKGRFTIS |
| | | | RDNAKNTLYLQMNSLRDEDTAVYYCATEYYWNIDNWGQGTLVTVSS |
| 17 | scFv 17 | DNA | CAGCCTGTGCTGACCCAGCCACCCTCTGTGTCTGCAGCCCTGGGGCAG |
| | | | AGGGTCACCATCTCCTGCACTGGAAGTAAGACCAACATCGGCAGTGGT |
| | | | TATGATGTACAATGGTACCAGCAGCTCCCAGGAAAGTCCCCTAAAACC |
| | | | ATCATTTATGGTAATAGCAATCGACCCTCGGGGGTCCCGGCTCGATTCT |
| | | | CTGGCTCCAAGTCAGGCAGCATCGCCACCCTGACCATCACTGGGATCC |
| | | | AGGCTGAGGATGAGGCTGATTATTACTGCCAGTCCTTTGATGACAATGT |
| | | | CGATGGTTACGTGTTCGGCTCAGGGACCCAGCTGACCATCCTCGGTGG |
| | | | TGGTTCCTCTAGATCTTCCTCCTCTGGTGGCGGTGGCTCGGGCGGTGGT |
| | | | GGGGAGGTGCCACTGGTGGAATCTGGGGGAGACCTGGTGAAGCCTGG |
| | | | GGGATCCCTGAGACTGTCCTGTGTGGCCTCTGGATTCACCTTCAGTAGC |
| | | | TACGGCATGAGCTGGGTCCGCCAGGCTCCAGGGAAGGGACTACAGTG |
| | | | GGTCGCATACATTAATGGTGGTGGAAGTAGTACATCCTACGCAGACGCT |
| | | | GTGAGGGGCCGATTCACCATCTCCAGAGACAACGCCGAGAACACGCT |
| | | | GTATCTGCAGATGAACAGCCTGAGAGCCGACGATACGGCTGTATATTAC |
| | | | TGTTCGAATTACTACGGTATCTACTGGGATTCTGACTACTGGGGCCAGG |
| | | | GAACCCTGGTCACCGTCTCCTCA |
| 60 | scFv 17 | AA | QPVLTQPPSVSAALGQRVTISCTGSKTNIGSGYDVQWYQQLPGKSPKTIIY |
| | | | GNSNRPSGVPARFSGSKSGSIATLTITGIQAEDEADYYCQSFDDNVDGYVF |
| | | | GSGTQLTILGGGSSRSSSSGGGGSGGGGEVPLVESGGDLVKPGGSLRLSCV |
| | | | ASGFTFSSYGMSWVRQAPGKGLQWVAYINGGGSSTSYADAVRGRFTISR |
| | | | DNAENTLYLQMNSLRADDTAVYYCSNYYGIYWDSDYWGQGTLVTVSS |
| 18 | scFv 18 | DNA | CAGTCTGCGCTGACTCAAACGGCCTCAGTGTCTGGGTCCCTGGGCCAG |
| | | | AAGGTCACCATCTCCTGCACTGGAAGCAGGTCCAACATCGGTGCAAAT |
| | | | TATGTGGGCTGGTATCAACAGGTCCCAGGAATAGGCCCTAGAACCGTC |
| | | | ATTTTTGGTTATAATTATCGACCTTCAGGGGTCCCCGATCGATTCTCTAG |
| | | | CTCCAAGTCAGGCAATTCAGCCACCCTGACCATCTCTGGGCTCCAGGC |
| | | | TGAGGACGAGGCTGAGTATTATTGTTCAACATGGGACAGCAGTCTCAA |
| | | | CGCTTACGTGTTCGGCTCAGGGACCCAGCTGACCGTCCTCGGCGGTGG |
| | | | TTCCTCTAGATCTTCCTCCTCTGGTGGCGGTGGCTCGGGCGGTGGTGG |
| | | | GGAGGTGCCACTGGTGGAATCTGGGGGAGACCTGGTGAAGCCTGGGG |
| | | | GGTCCTTGAGACTGTCCTGTGTGGCCTCTGGATTCACCCTCAGTAACTA |
| | | | TGGCATGAGCTGGGTCCGTCAGTCTCCAGGGAAGGGGCTGCAGTGGG |
| | | | TCGCAGCTATTAGGAGTGGTGGAAACACATACTACGCAGACGCTGTGA |
| | | | AGGGCCGATTCACCATCTCCAGAGACAACGCCAAGAACACGCTGTATC |
| | | | TGCAGATGAACAGCCTGAGAGCCGAGGACACGGCCGTGTATTACTGTG |
| | | | CGTCCCAATGGGGTGATTGGGGCCACTTTGAGTACTGGGGCCAGGGCA |
| | | | CCCTGGTCACCGTCTCCTCA |
| 61 | scFv 18 | AA | QSALTQTASVSGSLGQKVTISCTGSRSNIGANYVGWYQQVPGIGPRTVIFG |
| | | | YNYRPSGVPDRFSSSKSGNSATLTISGLQAEDEAEYYCSTWDSSLNAYVF |
| | | | GSGTQLTVLGGGSSRSSSSGGGGSGGGGEVPLVESGGDLVKPGGSLRLSC |
| | | | VASGFTLSNYGMSWVRQSPGKGLQWVAAIRSGGNTYYADAVKGRFTISR |
| | | | DNAKNTLYLQMNSLRAEDTAVYYCASQWGDWGHFEYWGQGTLVTVSS |
| 19 | scFv 19 | DNA | CAGGCTGTGCTGACTCAGCCGGCCTCAGTGTCGGGGCCCCTTGGCCAG |
| | | | AGGGTCACCATTTCCTGCTCTGGAAGCACGAACAACATCGGTATTGTT |
| | | | GGTGCGGCCTGGTACCAACAGTTTCCAGGAAAGGCCCCTAAACTCCTC |
| | | | GTATACAGTGATGGGAGTCGACCGTCAGGGGTCCCTGACCGGTTTTCC |
| | | | GGCTCCAACTCTGGCAACTCAGCCACCCTGACCATCAGTGGACTTCAG |
| | | | GCTGAGGACGAGGCTGATTATTACTGTCAGTCTGTTGATGCCACGCATG |
| | | | GTACTATTCTGTTCGGCGGAGGGACCCAGCTGACCGTCCTCGGTGGTG |
| | | | GTTCCTCTAGATCTTCCTCCTCTGGTGGCGGTGGCTCGGGCGGTGGTG |
| | | | GGGAGGTGCAGCTGGTGGAGACTGGGGGAGACCTGGTGAAGCCTGCG |
| | | | GGGTCCCTGAGACTGTCATGTGCGGCCTCTGGATTCCCCTTCAGTGGC |
| | | | TACAGTATGACCTGGGTCCGCCAGGCCCCTGAAAAGGGTCTGCAATTG |
| | | | GTCGCAGGTATTAACAGCGATGGAAGTCACACATACTACACAGACGAT |
| | | | GTGAAGGGCCGATTCACCGTCTCCAGAGACAACACCAAATACATACTG |
| | | | TATCTGCAGATGAACAGTCTGAGAGTCGAGGACACGGCCATATATTATT |
| | | | GTGGCTCTCATAGTTGGGGCAATTTTCACTACTGGGGCCAGGGAACCC |
| | | | TGGTCACCGTCTCCTCA |
| 62 | scFv 19 | AA | QAVLTQPASVSGPLGQRVTISCSGSTNNIGIVGAAWYQQFPGKAPKLLVYS |
| | | | DGSRPSGVPDRFSGSNSGNSATLTISGLQAEDEADYYCQSVDATHGTILFG |
| | | | GGTQLTVLGGGSSRSSSSGGGGSGGGGEVQLVETGGDLVKPAGSLRLSCA |
| | | | ASGFPFSGYSMTWVRQAPEKGLQLVAGINSDGSHTYYTDDVKGRFTVSR |
| | | | DNTKYILYLQMNSLRVEDTAIYYCGSHSWGNFHYWGQGTLVTVSS |
| 20 | scFv 20 | DNA | CAGTCTGTGCTGAATCAGCCACCTTCAGTATCAGTCTCTCTGGGACAG |
| | | | ACAGCAACCATCTCCTGCTCTGGAGAGAGTCTGAGTAAATATTATGCAC |
| | | | AATGGTTCCAGCAGAAGGCAGGCCAAGTCCCTGTGTTGGTCATATATA |
| | | | AGGACACTGAGCGGCCCTCTGGGATCCCTGACCGATTCTCCGGCTCCA |
| | | | GTTCAGGGAACACACACACCCTGACCATCAGCGGGGCTCGGGCCGAG |
| | | | GACGAGGCTGACTATTACTGCGAGTCAGAAGTCAGTCCTGATACTATTG |
| | | | TGTTCGGCGGAGGCACCCACCTGACCGTCCTCGGTGGTGGTTCCTCTA |
| | | | GATCTTCCTCCTCTGGTGGCGGTGGCTCGGGCGGTGGTGGGGAGGTAC |
| | | | AGCTGGTGGAATCTGGGGGAGACCTGGTGAAGCCTGGGGGATCCCTG |
| | | | AGACTGTCCTGTGTGGCCTCTGGATTCACCTTCAGTAGCTATGCCATGA |
| | | | GCTGGGTCCGCCAGGCTCCAGGGGAGGGGCTGCAGTGGGTCGCATAC |
| | | | ATTAACAGTGCTGGAAGTACCACATACTACGCAGACGCTGTGAAGGGC |
| | | | CGATTCACCATCTCCAGAGACAACGCCAGGAACACAGTGTATCTGCAG |
| | | | ATGAACAGCCTGAGAGCCGAGGACACAGCCGTGTATTACTGTGGCCGA |
| | | | TATTATTATGGTCTGGACTACTGGGGTCCTGGCACCTCAGTCTTCGTGT |
| | | | CCTCA |
| 63 | scFv 20 | AA | QSVLNQPPSVSVSLGQTATISCSGESLSKYYAQWFQQKAGQVPVLVIYKD |
| | | | TERPSGIPDRFSGSSSGNTHTLTISGARAEDEADYYCESEVSPDTIVFGGGT |
| | | | HLTVLGGGSSRSSSSGGGGSGGGGEVQLVESGGDLVKPGGSLRLSCVASG |
| | | | FTFSSYAMSWVRQAPGEGLQWVAYINSAGSTTYYADAVKGRFTISRDNA |
| | | | RNTVYLQMNSLRAEDTAVYYCGRYYYGLDYWGPGTSVFVSS |
| 21 | scFv 21 | DNA | CAGCCTGTGCTGACCCAGCCACCCTCGGTGTCAGTGTCTCTGGGACAG |
| | | | ACAGCAACCATCTCCTGCTCTGGAGAGAGTCTGAGTAAATATTATGCAC |
| | | | AATGGTTCCAGCAGAAGGCAGGCCAAGCCCCTGTGTTGGTCATATATA |
| | | | AGGACACTGAGCGGCCCTCTGGGATCCCTGACCGATTCTCTGGCTCCA |
| | | | GTTCAGGGAACACACACACCCTGACCATCAGCGGGGCTCGGGCCGAG |
| | | | GACGAGGCTGACTATTACTGCGAGTCAGCAGTCAGTAGTGATACTGCA |
| | | | GTGTTCGGCTCAGGGACCCAACTGACCGTCCTCGGCGGTGGTTCCTCT |
| | | | AGATCTTCCTCCTCTGGTGGCGGTGGCTCGGGCGGTGGTGGGGAGGTA |
| | | | CAGCTGGTGGAGTCTGGGGGAGATATGGTGAAGCCTGGGGGGTCCTTG |
| | | | AGACTGTCCTGTGTGGCCTCTGGATTCACCTTCAGTAGTTTCTGGATGT |
| | | | ACTGGGTCCGCCAGGCTCCTGGGAAGGGGCTGCAGTGGGTCGGAGGT |
| | | | ATTAATAGTGGTGGAAGTATTACAAGTTACGCAGACGCTGTGAAGGGC |
| | | | CGATTCACCATCTCCAGAGACAACGCCAGGAACACACTGTATCTGCAG |
| | | | ATGGACAGTCTGAGAGATGAAGACACAGCCGTCTACTACTGTGCGAAG |
| | | | CCGGATATATATGGGAGTTTTGACCACTGGGGCCAGGGCACCCTGGTC |
| | | | ACCGTCTCCTCA |
| 64 | scFv 21 | AA | QPVLTQPPSVSVSLGQTATISCSGESLSKYYAQWFQQKAGQAPVLVIYKDT |
| | | | ERPSGIPDRFSGSSSGNTHTLTISGARAEDEADYYCESAVSSDTAVFGSGTQ |
| | | | LTVLGGGSSRSSSSGGGGSGGGGEVQLVESGGDMVKPGGSLRLSCVASGF |
| | | | TFSSFWMYWVRQAPGKGLQWVGGINSGGSITSYADAVKGRFTISRDNAR |
| | | | NTLYLQMDSLRDEDTAVYYCAKPDIYGSFDHWGQGTLVTVSS |
| 22 | scFv 22 | DNA | CAGCCTGTGCTCACTCAGCCGCCCTCAGTGTCTGGGTCCCTGGGCCAG |
| | | | AGGGTCACCATCTCCTGCACTGGAAGCAGCTCCAATGTTGGTTATGGC |
| | | | GATTATGTGGGCTGGTACCAGCAGCTCCCAGGAACAAGCCCCAGAACC |
| | | | CTCATTTATTCTAGTAGTAGCCGACCCTCGGGAGTCCCCGATCGATTCT |
| | | | CTGGCTCCAGGTCAGGCAGCACAGCAACCCTGACCATCTCTGGGCTCC |
| | | | AGGCTGAAGATGAAGCCGATTATCACTGCTCATCCTATGACACCAGTCT |
| | | | CAATGGTTTCGTGTTCGGCTCAGGGACCCAGCTGACCATCCTCGGTGG |
| | | | TGGTTCCTCTAGATCTTCCTCCTCTGGTGGCGGTGGCTCGGGCGGTGGT |
| | | | GGGGAACTCACACTGCAGGAGTCAGGGCCAGGACTGGTGAAGCCCGC |
| | | | ACAGACCCTCTCTCTCACCTGTACTGTGTCCGGAGACTCCGTCACCAG |
| | | | CAGTCACTTCTGGCACTGGATCCGCCAGCGCCCTGGGAGGGGACTGG |
| | | | AATGGATGGGATACTGGTCAGACATTACGATTTACAGTCCAACATTCCA |
| | | | AGGACGCATCTCCATCACTGTTGACACGGCCAGGAACCAGTTCTCCCT |
| | | | GCATTTGAACTCCGTGACGACCGAGGACTCGGCCGTTTATTTCTGTGC |
| | | | AAGATATAATGGATACGTCGATTATTTAGACGTCTGGGGCCAGGGCACC |
| | | | CTGATCACCGTCTCCTCA |
| 65 | scFv 22 | AA | QPVLTQPPSVSGSLGQRVTISCTGSSSNVGYGDYVGWYQQLPGTSPRTLIY |
| | | | SSSSRPSGVPDRFSGSRSGSTATLTISGLQAEDEADYHCSSYDTSLNGFVFG |
| | | | SGTQLTILGGGSSRSSSSGGGGSGGGGELTLQESGPGLVKPAQTLSLTCTV |
| | | | SGDSVTSSHFWHWIRQRPGRGLEWMGYWSDITIYSPTFQGRISITVDTAR |
| | | | NQFSLHLNSVTTEDSAVYFCARYNGYVDYLDVWGQGTLITVSS |
| 23 | scFv 23 | DNA | GATATTGTCATGACACAGACGCCACTGTCCCTGTCCGTCAGTCCTGGA |
| | | | GAGCCGGCCTCCATCTCCTGCAAGGCCAGTCAGAGCCTCCTGCACAGT |
| | | | AACGGGAACACCTATTTGTATTGGTTCCGACAGAAGCCAGGCCAGTCT |
| | | | CCACAGCGCCTGATCTACTTGGTTTCCAACAGAGACGCTGGGGTCCCA |
| | | | GACAGGTTCAGTGGCAGCGGGTCAGGGACAGATTTCACCCTGAGAAT |
| | | | CAGCAGAGTAGAGGCTGATGATTCTGGAATTTATTACTGCGGCCAAGG |
| | | | TAAACAAGATCCTATCACCTTTGGCGAAGGGACACATCTGGAGATTAA |
| | | | AGGTGGTTCCTCTAGATCTTCCTCCTCTGGTGGCGGTGGCTCGGGCGG |
| | | | TGGTGGGGAACTCACACTGCAGGAGTCAGGGCCAGGACTGGTGAAGC |
| | | | CCTCACAGACCCTCTCTCTCACCTGTGTTGTGTCCGGAGGCTCCGTCA |
| | | | CCACCAGTGACTACTGGAACTGGCTCCGCCAGCGCCCTGGGAGGGGA |
| | | | CTGGAATACATGGGATACTGGAAAGGTCGGACTGAGTACAACCCGGCA |
| | | | TTCCAGGGACGCATCTCCATCACTGCTGACACGGCCAAGAACCAGTTC |
| | | | TCCCTGCAGTTAAACTCCGTGACCGCCGACGACACGGCCATTTATTATT |
| | | | GTGCACATTGGGGGTCAGGCAATCTTGACTATTGGGGCCCGGGAACCC |
| | | | TGGTCACCGTCTCCTCA |
| 66 | scFv 23 | AA | DIVMTQTPLSLSVSPGEPASISCKASQSLLHSNGNTYLYWFRQKPGQSPQR |
| | | | LIYLVSNRDAGVPDRFSGSGSGTDFTLRISRVEADDSGIYYCGQGKQDPIT |
| | | | FGEGTHLEIKGGSSRSSSSGGGGSGGGGELTLQESGPGLVKPSQTLSLTCV |
| | | | VSGGSVTTSDYWNWLRQRPGRGLEYMGYWKGRTEYNPAFQGRISITAD |
| | | | TAKNQFSLQLNSVTADDTAIYYCAHWGSGNLDYWGPGTLVTVSS |
| 24 | scFv 24 | DNA | CAGTCTGTGCTGAATCAGCCACCTTCTGTGTCTGGAGCCCTGGGGCAG |
| | | | ACCGTCACCATCTCCTGCTCTGGAAGTCACACCAACATCGGCAGTGGA |
| | | | TTTGATGTACAGTGGTACCAACAGCTCCCAGGAAAGTCCCCTCAAACT |
| | | | CTCGTCTATGCTAATATCAATCGACCCTCGGGGGTCCCGGTTCGATTCT |
| | | | CTGGCTCCAAGTCAGGCACCACAGCCACCCTGACCATCACTGGGATCC |
| | | | AGGCTGAGGATGAGGCTGATTATTACTGCCAGTCCTATGATGACAACGT |
| | | | CGATGGTTACGTGTTCGGCTCAGGGACCCAGCTGACCGTCCTTGGTGG |
| | | | TGGTTCCTCTAGATCTTCCTCCTCTGGTGGCGGTGGCTCGGGCGGTGGT |
| | | | GGGGAGGTCCAGCTGGTGCAGTCTGGGGGARACCTGGTGAAGCCTGG |
| | | | GGGGTCCCTGAGACTCTCCTGTGTGGCCTCTGGATTCACCCTCAGTAG |
| | | | TAACTATATGGACTGGGTCCGCCAGGCTCCAGGGAAGGGGCCTCAGTG |
| | | | GGTCGCACAAATTAGCACTGATGGAAGTAGGACATATTACGCAGACGC |
| | | | TGTGAAGGGCCGATTCACCATTTCCAGAGACACCGCCAAGAACACACT |
| | | | GTATCTGCAGATGAACAGTCTGAGAGCCGAGGACACGGCTATGTATTA |
| | | | CTGTGCAAAGAACGAATACTTGGCCCCTACGTACTGGGGCCAGGGCAC |
| | | | CCTGGTCACCGTCTCCTCA |
| 67 | scFv 24 | AA | QSVLNQPPSVSGALGQTVTISCSGSHTNIGSGFDVQWYQQLPGKSPQTLV |
| | | | YANINRPSGVPVRFSGSKSGTTATLTITGIQAEDEADYYCQSYDDNVDGY |
| | | | VFGSGTQLTVLGGGSSRSSSSGGGGSGGGGEVQLVQSGGXLVKPGGSLRL |
| | | | SCVASGFTLSSNYMDWVRQAPGKGPQWVAQISTDGSRTYYADAVKGRFT |
| | | | ISRDTAKNTLYLQMNSLRAEDTAMYYCAKNEYLAPTYWGQGTLVTVSS |
| 25 | scFv 25 | DNA | GATATCGTCATGACACAGACTCCGCTGTCCCTGTCCGTCAGCCCTGGA |
| | | | GAGACGGCCTCCATCTCCTGCAAGGCCAGTCAGAGCCTCCTGCACAGT |
| | | | AACGGGAACACCTATTTGAATTGGTTCCGACAGAAGCCAGGCCAGTCT |
| | | | CCACAGGTCCTGATCTATCAGGTCTCCAACAGAGACACTGGGGTCCCA |
| | | | GACAGGTTCAGTGGCAGCGGGTCAGGGACAGATTTCACCCTGAGAAT |
| | | | CAGTAGAGTGGAGGCTGACGATGCTGGAGTTTATTATTGCGGGCAAGG |
| | | | TATACAATATCCTCCGACGTTCGGAGCAGGAACCAAGGTGGAGCTCAA |
| | | | AGGTGGTTCCTCTAGATCTTCCTCCTCTGGTGGCGGTGGCTCGGGCGG |
| | | | TGGTGGGGAGGAGCAACTGGTGGAGTTTGGGGGAGACCTGGTGAAGC |
| | | | CTGGGGGGTCCTTGAGACTCTCCTGTGTGGCCTCTGGATTCACCTTCA |
| | | | GTAGTCTTTCCATGAACTGGATCCGCCAGGCTCCAGGGAAGGGGCTGC |
| | | | AGTGGGTCTCACAAATTAGCCCTGATGGAACTATCACAGGCTACACAG |
| | | | ACGCTGTGAAGGGCCGATTCACCATCTCCAGAGACAATGCCAAGAAC |
| | | | ACGCTGTATCTTCAGATGAACAGCCTGAGAGATGAAGACACGGCAATA |
| | | | TATTACTGTGCGAAGAGTCGATATACGGATACGTACCCCTTTGACTACT |
| | | | GGGGCCCGGGAACCCTGGTCACCGTCTCCTCA |
| 68 | scFv 25 | AA | DIVMTQTPLSLSVSPGETASISCKASQSLLHSNGNTYLNWFRQKPGQSPQ |
| | | | VLIYQVSNRDTGVPDRFSGSGSGTDFTLRISRVEADDAGVYYCGQGIQYP |
| | | | PTFGAGTKVELKGGSSRSSSSGGGGSGGGGEEQLVEFGGDLVKPGGSLRL |
| | | | SCVASGFTFSSLSMNWIRQAPGKGLQWVSQISPDGTITGYTDAVKGRFTIS |
| | | | RDNAKNTLYLQMNSLRDEDTAIYYCAKSRYTDTYPFDYWGPGTLVTVSS |
| 26 | scFv 26 | DNA | TCCAGTGTGCTGACTCAGCCTCCCTCGGTATCAGTGTCTCTGGGACAG |
| | | | ACAGCAACCATCTCCTGCTCTGGAGAGAGTCTGAGTAAATATTATACTC |
| | | | AATGGTTCCAGCAGAAGGCAGGCCAAGCCCCTATCTTGGTCATTTATAA |
| | | | GGACACTGAGCGGCCCTCTGGGATCCCTGACCGGTTCTCTGGCTCCAG |
| | | | TTCAGGCAACACACACACCCTGACCATCAGCGGGGCTCGGGCCGAGG |
| | | | ACGAGGCTGACTATTATTGCGAGTCATTAGTCGATTCTGATGCTTACATG |
| | | | TTCGGCTCTGGAACCCAGCTGACCGTCCTTGGCGGTGGTTCCTCTAGA |
| | | | TCTTCCTCCTCTGGTGGCGGTGGCTCGGGCGGTGGTGGGGAGGTGCAG |
| | | | CTGGTGGAGACTGGGGGAGACCTGGTGAAGCCTGGGGGGTCCCTGAG |
| | | | ACTCTCCTGTGTGGGCTCTGGATTCACCTTCAGTAGCTACTGGATGAGC |
| | | | TGGGTCCGCCAGGCTCCAGGGAAGGGGCTACAGTGGATTGCAGATATT |
| | | | AGCGATAGTGGAAGTAGCACAGACTATGCAGACGCTGTGAAGGGCCG |
| | | | ATTCACCATCTCCAGAGACAATGCCAAGAACACACTGTATCTGCAGAT |
| | | | GAACAGCCTGAGAGCCGAGGACACGGCCATGTATTACTGTGCAAGGG |
| | | | GTCTGGGTATGGACTACTGGGGCCATGGCACCTCACTCTTCGTGTCCTC |
| | | | A |
| 69 | scFv 26 | AA | SSVLTQPPSVSVSLGQTATISCSGESLSKYYTQWFQQKAGQAPILVIYKDT |
| | | | ERPSGIPDRFSGSSSGNTHTLTISGARAEDEADYYCESLVDSDAYMFGSGT |
| | | | QLTVLGGGSSRSSSSGGGGSGGGGEVQLVETGGDLVKPGGSLRLSCVGSG |
| | | | FTFSSYWMSWVRQAPGKGLQWIADISDSGSSTDYADAVKGRFTISRDNA |
| | | | KNTLYLQMNSLRAEDTAMYYCARGLGMDYWGHGTSLFVSS |
| 27 | scFv 27 | DNA | CAGCCTGTGCTGACCCAGCCACCCTCGGTATCAGTGTCTCTGGGACAG |
| | | | ACAGCAACCATCTCCTGCTCTGGAGAGAGTCTGAGTAAATATTATGCAC |
| | | | AATGGTTCCAGCAGAAGGCAGGCCAAGCCCCTGTGTTGGTCATATATA |
| | | | AGGACACTGAGCGGCCCTCTGGGATCCCTGACCGATTCTCTGGCTCCA |
| | | | GTTCAGGGAACACACACACCCTGACCATCACTGGGATCCAGGCTGAG |
| | | | GATGAGGGTGATTATCACTGCCACTCCTATGATGAGAACCTCGATGGTT |
| | | | ACGTGTTCGGCTCAGGAACCCAACTGACCGTCCTTGGCGGTGGTTCCT |
| | | | CTAGATCTTCCTCCTCTGGTGGCGGTGGCTCGGGCGGTGGTGGGGAGG |
| | | | TGCAGCTGGTGGAGTCTGGGGGAGACCTGGTGAAGCCTGGGGGGTCT |
| | | | CTGAGACTCTCCTGTGTGGGCTCTGGATTCACCTTCAGTAGCTACTGGA |
| | | | TGAGCTGGGTCCGCCAGGCTCCAGGGAAGGGGCTACAGTGGATTGCA |
| | | | GATATTAGCGATAGTGGAAGTAGCACAGACTATGCAGACGCTGTGAAG |
| | | | GGCCGATTCACCATCTCCAGAGACAATGCCAAGAACACACTGTATCTG |
| | | | CAGATGAACAGCCTGAGAGCCGAGGACACGGCCATGTATTACTGTGCA |
| | | | AGGGGTCTGGGTATGGACTACTGGGGCCATGGCACCTCACTCTTCGTG |
| | | | TCCTCA |
| 70 | scFv 27 | AA | QPVLTQPPSVSVSLGQTATISCSGESLSKYYAQWFQQKAGQAPVLVIYKDT |
| | | | ERPSGIPDRFSGSSSGNTHTLTITGIQAEDEGDYHCHSYDENLDGYVFGSG |
| | | | TQLTVLGGGSSRSSSSGGGGSGGGGEVQLVESGGDLVKPGGSLRLSCVGS |
| | | | GFTFSSYWMSWVRQAPGKGLQWIADISDSGSSTDYADAVKGRFTISRDN |
| | | | AKNTLYLQMNSLRAEDTAMYYCARGLGMDYWGHGTSLFVSS |
| 28 | scFv 28 | DNA | CAGTCTGCCCTGACTCAACCTTCCTCGGTATCAGTGTCTCTGGGACAG |
| | | | ACAGCAACCATCTCCTGCTCTGGAGAGAGTCTGAATAAATATTATGTAC |
| | | | AGTGGTTCCAGCAGAAGGCAGGCCAAGTCCCTATATTGGTCATATATAG |
| | | | GGACACTGAGCGGCCCTCTGGGATCCCTGACCGATTCTCTGGCTCCAG |
| | | | TTCAGGGAACACACACACCCTGACCATCACCGGGACTCGGGCCGAGG |
| | | | ACGAGGCTGACTATTACTGCGAGTCACTAGTCAATGATAATACTTACGT |
| | | | GTTCGGCTCAGGGACCCAACTGACCGTCCTTGGCGGTGGTTCCTCTAG |
| | | | ATCTTCCTCCTCTGGTGGCGGTGGCTCGGGCGGTGGTGGGGAGGTGCA |
| | | | GCTGGTGGAGACTGGGGGAGACCTGGTGAAGCCTGGGGGGTCCCTGA |
| | | | GACTCTCCTGTGTGGGCTCTGGATTCACCTTCAGTAGCTACTGGATGAG |
| | | | CTGGGTCCGCCAGGCTCCAGGGAAGGGGCTACAGTGGATTGCAGATAT |
| | | | TAGCGATAGTGGAAGTAGCACAGACTATGCAGACGCTGTGAAGGGCCG |
| | | | ATTCACCATCTCCAGAGACAATGCCAAGAACACACTGTATCTGCAGAT |
| | | | GAACAGCCTGAGAGCCGAGGACACGGCCATGTATTACTGTGCAAGGG |
| | | | GTCTGGGTATGGACTACTGGGGCCATGGCACCTCACTCTTCGTGTCCTC |
| | | | A |
| 71 | scFv 28 | AA | QSALTQPSSVSVSLGQTATISCSGESLNKYYVQWFQQKAGQVPILVIYRDT |
| | | | ERPSGIPDRFSGSSSGNTHTLTITGTRAEDEADYYCESLVNDNTYVFGSGT |
| | | | QLTVLGGGSSRSSSSGGGGSGGGGEVQLVETGGDLVKPGGSLRLSCVGSG |
| | | | FTFSSYWMSWVRQAPGKGLQWIADISDSGSSTDYADAVKGRFTISRDNA |
| | | | KNTLYLQMNSLRAEDTAMYYCARGLGMDYWGHGTSLFVSS |
| 29 | scFv 29 | DNA | CAGTCTGTGCTGAATCAGCCGCCTTCAGTATCAGTGTCTCTGGGACAG |
| | | | ACAGCAACCATCTCCTGCTCTGGAGAGAGTCTGAATAAATATTATACAC |
| | | | AGTGGTTCCAGCAGAAGGCAGGCCAAGCCCCTGTGTTGGTCATATATA |
| | | | AGGACACTGAGCGGCCCTCTGGGATCCCTGACCGATTCTCCGGATCCA |
| | | | GTTCAGGGAACACACACACCCTGACCATCAGCGGGGCTCGGGCCGAG |
| | | | GACGAGGCTGACTATTACTGCGAGTCAGTAGACAGTACTGATACTTAC |
| | | | GTGTTCGGCTCAGGGACCCAGCTGACCATCCTCGGTGGTGGTTCCTCT |
| | | | AGATCTTCCTCCTCTGGTGGCGGTGGCTCGGGCGGTGGTGGGGAGGTA |
| | | | CATCTGGTGGAATCTGGGGGAGACTTGGTGAAGCCTGGGGGGTCCCTG |
| | | | AGACTCTCCTGTGTGGGCTCTGGATTCACCTTCAGTAGCTACTGGATGA |
| | | | GCTGGGTCCGCCAGGCTCCAGGGAAGGGGCTACAGTGGATTGCAGAT |
| | | | ATTAGCGATAGTGGAAGTAGCACAGACTATGCAGACGCTGTGAAGGGC |
| | | | CGATTCACCATCTCCAGAGACAATGCCAAGAACACACTGTATCTGCAG |
| | | | ATGAACAGCCTGAGAGCCGAGGACACGGCCATGTATTACTGTGCAAGG |
| | | | GGTCTGGGTATGGACTACTGGGGCCATGGCACCTCACTCTTCGTGTCCT |
| | | | CA |
| 72 | scFv 29 | AA | QSVLNQPPSVSVSLGQTATISCSGESLNKYYTQWFQQKAGQAPVLVIYKD |
| | | | TERPSGIPDRFSGSSSGNTHTLTISGARAEDEADYYCESVDSTDTYVFGSG |
| | | | TQLTILGGGSSRSSSSGGGGSGGGGEVHLVESGGDLVKPGGSLRLSCVGS |
| | | | GFTFSSYWMSWVRQAPGKGLQWIADISDSGSSTDYADAVKGRFTISRDN |
| | | | AKNTLYLQMNSLRAEDTAMYYCARGLGMDYWGHGTSLFVSS |
| 30 | scFv 30 | DNA | CAGTCTGTCTTGACTCAGCCAGCCTCGGTATCAGTGTCTCTGGGACAG |
| | | | ACAGCAACCATCTCCTGCTCTGGAGAGAGTCTGAGTAAATATTATACAC |
| | | | AGTGGTTCCAGCAGAAGGCAGGCCAGGCCCCTGTGTTGGTCATCTATA |
| | | | AGGACACTGAGCGGCCCTCTGGGATCCCTGACCGATTCTCTGGCTCCA |
| | | | GCTCAGGGAACACGCACACCCTGACCATCAGCGGGGCTCAGGCCGAG |
| | | | GATGAGGCTGATTATTACTGCCAGTCCTATGATGACAACGTCGATGGTT |
| | | | ACGTGTTCGGCTCTGGGACCCAACTGACCGTCCTCGGTGGTGGTTCCT |
| | | | CTAGATCTTCCTCCTCTGGTGGCGGTGGCTCGGGCGGTGGTGGGGAGG |
| | | | TACAGCTGGTGGAATCTGGGGGAGACCTGGTGAAGCCTGGGGGGTCC |
| | | | CTGAGACTCTCCTGTGTGGGCTCTGGATTCACCTTCAGTAGCTACTGGA |
| | | | TGAGCTGGGTCCGCCAGGCTCCAGGGAAGGGGCTACAGTGGATTGCA |
| | | | GATATTAGCGATAGTGGAAGTAGCACAGACTATGCAGACGCTGTGAAG |
| | | | GGCCGATTCACCATCTCCAGAGACAATGCCAAGAACACACTGTATCTG |
| | | | CAGATGAACAGCCTGAGAGCCGAGGACACGGCCATGTATTACTGTGCA |
| | | | AGGGGTCTGGGTATGGACTACTGGGGCCATGGCACCTCACTCTTCGTG |
| | | | TCCTCA |
| 73 | scFv 30 | AA | QSVLTQPASVSVSLGQTATISCSGESLSKYYTQWFQQKAGQAPVLVIYKD |
| | | | TERPSGIPDRFSGSSSGNTHTLTISGAQAEDEADYYCQSYDDNVDGYVFG |
| | | | SGTQLTVLGGGSSRSSSSGGGGSGGGGEVQLVESGGDLVKPGGSLRLSCV |
| | | | GSGFTFSSYWMSWVRQAPGKGLQWIADISDSGSSTDYADAVKGRFTISR |
| | | | DNAKNTLYLQMNSLRAEDTAMYYCARGLGMDYWGHGTSLFVSS |
| 31 | scFv 31 | DNA | CAGTCTGCCCTGACTCAACCTTCCTCGGTATCAGTGTCTCTGGGACAG |
| | | | ACAGCAACCATCTCCTGCTCTGGAGAGAGTCTGACTAAATATTATGTAC |
| | | | AATGGTTCCAACAGAAGGCAGGCCAAGCCCCTGTATTGGTCATATATAG |
| | | | GGACACTGAGCGGCCCTCTGGGATCCCTGACCGATTCTCTGGCTCCAG |
| | | | TTCAGGTAATACACACACCCTGACCATCAGTGGGGCTCGGGCCGAGGA |
| | | | CGAGGCTGACTATTATTGCGAGTCAATGGACGGTCGTGAGGCTTACGT |
| | | | GTTCGGCTCAGGGACCCAACTGACCGTCCTCGGCGGTGGTTCCTCTAG |
| | | | ATCTTCCTCCTCTGGTGGCGGTGGCTCGGGCGGTGGTGGGGAGGTACA |
| | | | GCTGGTGGAGTCTGGGGGAGACCTGGTGAAGCCGGGGGGGTCCCTGA |
| | | | GACTGTCCTGTGTGGCCTCTGGATTCACCTTCAGTAGCTACTGGATGAG |
| | | | CTGGGTCCGCCAGGCTCCAGGGAAGGGGCTACAGTGGATTGCAGATAT |
| | | | TAGCGATAGTGGAAGTAGCACAGACTATGCAGACGCTGTGAAGGGCCG |
| | | | ATTCACCATCTCCAGAGACAATGCCAAGAACACACTGTATCTGCAGAT |
| | | | GAACAGCCTGAGAGCCGAGGACACGGCCATGTATTACTGTGCAAGGG |
| | | | GTCTGGGTATGGACTACTGGGGCCATGGCACCTCACTCTTCGTGTCCTC |
| | | | A |
| 74 | scFv 31 | AA | QSALTQPSSVSVSLGQTATISCSGESLTKYYVQWFQQKAGQAPVLVIYRDT |
| | | | ERPSGIPDRFSGSSSGNTHTLTISGARAEDEADYYCESMDGREAYVFGSGT |
| | | | QLTVLGGGSSRSSSSGGGGSGGGGEVQLVESGGDLVKPGGSLRLSCVASG |
| | | | FTFSSYWMSWVRQAPGKGLQWIADISDSGSSTDYADAVKGRFTISRDNA |
| | | | KNTLYLQMNSLRAEDTAMYYCARGLGMDYWGHGTSLFVSS |
| 32 | scFv 32 | DNA | CAGTCTGTGTTGACTCAACCAGCCTCGGTATCAGTGTCTCTGGGACAG |
| | | | ACAGCAACCATCTCCTGCTCTGGAGAGAGTCTGAGTAAATATTATGCAC |
| | | | AATGGTTCCAGCAGAAGGCAGGCCAAGCCCCTGTGTTGGTCATATATA |
| | | | AGGACACTGAGCGGCCCTCTGGGATCCCTGACCGATTCTCTGGCTCCA |
| | | | GTTCAGGGAACACACACACCCTGACCATCACTGGGATCCAGGCTGCG |
| | | | GATGAGGCTGATTATTACTGCCAGTCCTATGATGACAACCTCGATGGTT |
| | | | ACGTGTTCGGCTCAGGAACCCAGCTGACCGTCCTTGGTGGTGGTTCCT |
| | | | CTAGATCTTCCTCCTCTGGTGGCGGTGGCTCGGGCGGTGGTGGGGAGG |
| | | | TACAGCTGGTGGAGTCTGGGGGAGACCTGGTGAAGCCTGGGGGGTCC |
| | | | CTGAGACTCTCCTGTGTGGGCTCTGGATTCACCTTCAGTAGCTACTGGA |
| | | | TGAGCTGGGTCCGCCAGGCTCCAGGGAAGGGGCTACAGTGGATTGCA |
| | | | GATATTAGCGATAGTGGAAGTAGCACAGACTATGCAGACGCTGTGAAG |
| | | | GGCCGATTCACCATCTCCAGAGACAATGCCAAGAACACACTGTATCTG |
| | | | CAGATGAACAGCCTGAGAGCCGAGGACACGGCCATGTATTACTGTGCA |
| | | | AGGGGTCTGGGTATGGACTACTGGGGCCATGGCACCTCACTCTTCGTG |
| | | | TCCTCA |
| 75 | scFv 32 | AA | QSVLTQPASVSVSLGQTATISCSGESLSKYYAQWFQQKAGQAPVLVIYKD |
| | | | TERPSGIPDRFSGSSSGNTHTLTITGIQAADEADYYCQSYDDNLDGYVFGS |
| | | | GTQLTVLGGGSSRSSSSGGGGSGGGGEVQLVESGGDLVKPGGSLRLSCVG |
| | | | SGFTFSSYWMSWVRQAPGKGLQWIADISDSGSSTDYADAVKGRFTISRD |
| | | | NAKNTLYLQMNSLRAEDTAMYYCARGLGMDYWGHGTSLFVSS |
| 33 | scFv 33 | DNA | GATATTGTCATGACACAGACGCCACTGTCCCTGTCCGTCAGCCCTGGA |
| | | | GAGCCGGCCTCCATCTCCTGCAAGGCCAGTCAGAGCCTCCTGCACAGT |
| | | | GACGGGAACACCTATTTGCATTGGTACCTGCAAAAGCCAGGCCAGTCT |
| | | | CCACAGCTTCTGATCTACTCGGTTTCCAACCGCTTCACTGGCGTGTCAG |
| | | | ACAGGTTCAGTGGCAGCGGGTCAGGGACAGATTTCACCCTGAGAATC |
| | | | AGCAGAGTGGAGGCTGACGATACTGGAGTTTATTACTGCGGGCAAGGT |
| | | | ACACACCTCCCGTTCACTTTTGGCCAAGGGACCAAACTGGAGATCAAA |
| | | | GGTGGTTCCTCTAGATCTTCCTCCTCTGGTGGCGGTGGCTCGGGCGGT |
| | | | GGTGGGGAGGTACAGCTGGTGGAGTCTGGGGGAGACCTGGTGAAGCC |
| | | | TGGGGGGTCCCTGAGACTCTCCTGTGCAGCCTCTGGATTCACCTTCAG |
| | | | TCACTACGACATGAACTGGGTCCGCCAGGCTCCTGGGAAGGGGCTGC |
| | | | AGTGGGTCGCATCTATTGCCTATGATGGATCTAACACATACTACACTGA |
| | | | CGCTGTGAAGGGCCGATTCACCATCTCCAGAGACAACGCCAAGAATAT |
| | | | ACTGTTTTTGCTGGTGAATAGCCTGAGGGCCGAGGACACGGCCATATAT |
| | | | TATTGTGCGCGGGGGCTGGTCTACTGGGGCCAGGGAACTTTGGTCACC |
| | | | GTCTCCTCA |
| 76 | scFv 33 | AA | DIVMTQTPLSLSVSPGEPASISCKASQSLLHSDGNTYLHWYLQKPGQSPQL |
| | | | LIYSVSNRFTGVSDRFSGSGSGTDFTLRISRVEADDTGVYYCGQGTHLPFT |
| | | | FGQGTKLEIKGGSSRSSSSGGGGSGGGGEVQLVESGGDLVKPGGSLRLSC |
| | | | AASGFTFSHYDMNWVRQAPGKGLQWVASIAYDGSNTYYTDAVKGRFTIS |
| | | | RDNAKNILFLLVNSLRAEDTAIYYCARGLVYWGQGTLVTVSS |
| 34 | scFv 34 | DNA | TCCTATGAGCTGACTCAGCCACCATCTGTGTCTGCGGCCCTGGGACAG |
| | | | AGGGTCACCATCTCCTGCACTGGAACTAATACCAACATCGGCAGTGGT |
| | | | TATAGTGTACAATGGTACCAGCAGCTCCCAGGAGAGTCCCCAAAACCT |
| | | | ATCATTTATGGTAGTAGTAATCGACCCTCGGGGGTCCCGGCTCGATTCT |
| | | | CTGGCTCCAAGTCAGGCAGCACAGGCACCCTGACCATCACTGGGATCC |
| | | | AGGCTGAGGATGAGGCTGATTACCACTGCCAGTCCTATGATGACAGCC |
| | | | TCGATGGCCATGCTGTATTCGGCGGAGGGACCCAGCTGACCATCCTCG |
| | | | GCGGTGGTTCCTCTAGATCTTCCTCCTCTGGTGGCGGTGGCTCGGGCG |
| | | | GTGGTGGGGAGGTACAGCTGGTGGAGTCTGGGGGAGACCTGGTGAAG |
| | | | CCTGGGGGGTCCCTGAGACTCTCCTGTGTGGCCTCTGGATTAACCTTC |
| | | | AGTGACAATTACATGTCCTGGATCCGTCAGGTTCCAGGGAAGGGCCTG |
| | | | CAGTGGGTCTCACGAATTAGCACTGATGGAAGAGGCACAAGTTACGCA |
| | | | GACGCTGTGAAGGGCCGATTCACCATCTCCAGAGACAATGCCAAGAA |
| | | | CACGCTGTATCTGCAGATGAACAGCCTGAGAGACGAGGACACGGCCG |
| | | | TGTATTACTGTGCAAGGCGGTCTGGTATTTGGGGGTTTGACTACTGGGG |
| | | | CCAGGGCACCCTGGTCACCGTCTCCTCT |
| 77 | scFv 34 | AA | SYELTQPPSVSAALGQRVTISCTGTNTNIGSGYSVQWYQQLPGESPKPIIY |
| | | | GSSNRPSGVPARFSGSKSGSTGTLTITGIQAEDEADYHCQSYDDSLDGHAV |
| | | | FGGGTQLTILGGGSSRSSSSGGGGSGGGGEVQLVESGGDLVKPGGSLRLS |
| | | | CVASGLTFSDNYMSWIRQVPGKGLQWVSRISTDGRGTSYADAVKGRFTIS |
| | | | RDNAKNTLYLQMNSLRDEDTAVYYCARRSGIWGFDYWGQGTLVTVSS |
| 35 | scFv 35 | DNA | TCCAGTGTGCTGACTCAGCCTCCTTCGGTATCAGTGTCTCTGGGACAG |
| | | | ACAGCAACCATCTCCTGCTCTGGAGAGAGTCTGAGTGAATATTATGCA |
| | | | CAATGGTTCCAGCAGAAGGCAGGCCAAGCCCCTGTATTGGTCATATATA |
| | | | AGGACACTGAGCGGGCCTCTGGAATCCCTGACCGATTCTCTGGCTCCA |
| | | | GTTCGGGGAACACACACACCCTGACCATCAGCGGGGCTCGGGCCGAG |
| | | | GACGAGGCTGACTATTACTGCGAGTCAGCAGTCAATAGTGATGGTATTG |
| | | | TGTTCGGCGGAGGCACCCATCTGACCGTCCTCGGCGGTGGTTCCTCTA |
| | | | GATCTTCCTCCTCTGGTGGCGGTGGCTCGGGCGGTGGTGGGGAGGTAC |
| | | | AGCTGGTGGAGTCTGGGGGAGACCTGGTGAAGCCTGCGGGGTCCCTG |
| | | | AGACTGTCCTGTGTGACCTCTGGATTCACCTTCAGCAGCTATGCCATGA |
| | | | GCTGGGTCCGCCAGGCTCCTGGGAAGGGGCTGCAGTGGGTCGGAAGT |
| | | | ATTAACAGTGGTGGAAGTACCACAAGCTACGCAGACGCTGTGAAGGG |
| | | | CCGATTCACCATCTCCAGAGACAACGCCAAGAACACAGTGTATCTGCA |
| | | | GATGAACAGCCTGAGAGCCGAGGACACGGCCGTGTATTACTGTGCCCG |
| | | | GTGGTGGGACTTTGAGTACTGGGGCCAGGGCACCCTGGTCACCGTCTC |
| | | | CTCA |
| 78 | scFv 35 | AA | SSVLTQPPSVSVSLGQTATISCSGESLSEYYAQWFQQKAGQAPVLVIYKDT |
| | | | ERASGIPDRFSGSSSGNTHTLTISGARAEDEADYYCESAVNSDGIVFGGGT |
| | | | HLTVLGGGSSRSSSSGGGGSGGGGEVQLVESGGDLVKPAGSLRLSCVTSG |
| | | | FTFSSYAMSWVRQAPGKGLQWVGSINSGGSTTSYADAVKGRFTISRDNA |
| | | | KNTVYLQMNSLRAEDTAVYYCARWWDFEYWGQGTLVTVSS |
| 36 | scFv 36 | DNA | CAGTCTGTGCTGACTCAGCCGACCTCAGTGTCGGGGTCCCTTGGCCAG |
| | | | AGGGTCACCATCTCCTGCTCTGGAAGCACGAACAACATCGGTATTGTT |
| | | | GGTGCGAGCTGGTACCAACAGCTCCCAGGAAAGGCCCCTAAACTCCT |
| | | | CGTGTACAGTGATGGGGATCGACCGTCAGGGGTCCCTGACCGGTTTTC |
| | | | CGGCTCCAACTCTGGCAACTCAGACACCCTGACCATCACTGGGCTTCA |
| | | | GGCTGAGGACGAGGCTGATTATTACTGCCAGTCCTTTGATACCACGCTT |
| | | | GATGCTTACGTGTTCGGCTCAGGAACCCAGCTGACCGTCCTTGGTGGT |
| | | | GGTTCCTCTAGATCTTCCTCCTCTGGTGGCGGTGGCTCGGGCGGTGGT |
| | | | GGGGAGGAGCAACTGGTGGAGTTTGGGGGAGGCCTGGTGAGGCCTGG |
| | | | GGGATCCCTGAGACTGTCCTGTGTGGCCTCTGGATTCACCTTCAGTAGC |
| | | | TATGGCATGACCTGGGTCCGTCAGTCTCCAGGGAAGGGGCTGCAGTGG |
| | | | GTCGCTACTATTAGCGATACCGGAAGCACATACTACGCAGACACTGTGA |
| | | | AGGGCCGATTCAGCATCTCCAGAGACAACGCCAAGAACACGCTGTATC |
| | | | TGCAGATGAACAGTCTGAGAGCCGACGACACGGCCCTGTATTATTGTA |
| | | | CGAAGGACCTCGGGGTGTGGGGTATGGACAACTGGGGTCCTGGCACC |
| | | | TCACTCTTCGTGTCCTCA |
| 79 | scFv 36 | AA | QSVLTQPTSVSGSLGQRVTISCSGSTNNIGIVGASWYQQLPGKAPKLLVYS |
| | | | DGDRPSGVPDRFSGSNSGNSDTLTITGLQAEDEADYYCQSFDTTLDAYVF |
| | | | GSGTQLTVLGGGSSRSSSSGGGGGGGGEEQLVEFGGGLVRPGGSLRLSC |
| | | | VASGFTFSSYGMTWVRQSPGKGLQWVATISDTGSTYYADTVKGRFSISRD |
| | | | NAKNTLYLQMNSLRADDTALYYCTKDLGVWGMDNWGPGTSLFVSS |
| 37 | scFv 37 | DNA | TCCAGTGTGCTGACTCAGCCTCCCTCAGTATCAGTGTCTCTGGGACAG |
| | | | ACAGCAACCATCTCCTGCTCTGGAGAGAGTCTGAATGACTATTATGCAC |
| | | | AATGGTTCCAGCAGAAGGCAGGCCAAGTCCCTGTGTTGGTCATATATC |
| | | | GGGACACTGAGCGGCCCTCTGGGATCCCTGACCGATTCTCCGGCTCCA |
| | | | GTTCAGGGAACACACACACCCTGACCATCAGCGGGGCTCGGGCCGAG |
| | | | GACGAGGCTGACTATTACTGCGAGTCAGAAGTCAGTGCTGGTGGTGCT |
| | | | GTATTCGGCGAAGGCACCCACCTGACCGTCCTCGGTGGTGGTTCCTCT |
| | | | AGATCTTCCTCCTCTGGTGGCGGTGGCTCGGGCGGTGGTGGGGAGGTG |
| | | | CAGCTGGTGGAGACTGGGGGAGACCTGGTGAAGCCGGGGGGGTCCCT |
| | | | GAGACTGTCCTGTGTGGCCTCTGGATTCACCCTCAGAGACTACAGCAT |
| | | | GACCTGGGTCCGCCAGACTCCTGAGAAGGGGCTGCAGTTGGTCGCAG |
| | | | GTATTAGCACCGGTGGAAGTACCACATACTACATAGACGCTGTGAAGG |
| | | | GCCGATTCACCATCTCCAGAGACAACGCCAAGAACACAGTGTATCTGC |
| | | | AGATGAACAGCCTGAGAGCCGAGGACACGGCCATGTATCACTGTACAA |
| | | | CCACGGCCTTAGGTTACTGGGGCCAGGGCACCCTGGTCACTGTCTCCT |
| | | | CA |
| 80 | scFv 37 | AA | SSVLTQPPSVSVSLGQTATISCSGESLNDYYAQWFQQKAGQVPVLVIYRDT |
| | | | ERPSGIPDRFSGSSSGNTHTLTISGARAEDEADYYCESEVSAGGAVFGEGT |
| | | | HLTVLGGGSSRSSSSGGGGSGGGGEVQLVETGGDLVKPGGSLRLSCVASG |
| | | | FTLRDYSMTWVRQTPEKGLQLVAGISTGGSTTYYIDAVKGRFTISRDNAK |
| | | | NTVYLQMNSLRAEDTAMYHCTTTALGYWGQGTLVTVSS |
| 38 | scFv 38 | DNA | TCCAGTGTGCTGACTCAGCCTCCCTCAGTATCAGTCTCTCTGGGACAG |
| | | | ACAGCAACCATCTCCTGCTCTGGAGAGAGTCTGAGTGAGTATTATGCA |
| | | | CAATGGTTCCAGCAGAAGGCAGGCCAAGCCCCTGTGTTGGTCATATAT |
| | | | AAGGACACTGAGCGGCCCTCTGGGATCCCTGACCGATTCTCCGGCTCC |
| | | | AGTTCAGGGAACACACACACCCTGACCATCAGCGGGGCTCGGGCCGA |
| | | | GGACGAGGCTGACTATTACTGCGCGTCAGCAGACAGTAGTGCTATTCC |
| | | | TGTGTTCGGCGGAGGCACCCATCTGACCGTCCTCGGCGGTGGTTCCTC |
| | | | TAGATCTTCCTCCTCTGGTGGCGGTGGCTCGGGCGGTGGTGGGGAGGT |
| | | | GCAGCTGGTGGAGTCTGGGGGAGACCTGGTGAAGCCTGGGGGGTCCC |
| | | | TGAGACTCTCCTGTGTGGCCTCAGGATTCAATTTCGGAAACTATGCCGT |
| | | | GAGCTGGGTCCGCCAGGCTCCTGGGAAGGGACTGCAGTGGGTCGCAG |
| | | | GTATCAGTAACGATGGAACTATAACAGGCTACACAGATGTTGTGGAGG |
| | | | GCCGTTTCATCATCTCCAGAGACAATGCCAAGAACACGCTGTATCTACA |
| | | | GATGAACAGCCTGAGAGCCGAGGACATGGCTATATATTATTGTGTGGCT |
| | | | GGGGGCTTTCTTGACTACTGGGGCCAGGGAACCCTGGTCACCGTCTCC |
| | | | TCA |
| 81 | scFv 38 | AA | SSVLTQPPSVSVSLGQTATISCSGESLSEYYAQWFQQKAGQAPVLVIYKDT |
| | | | ERPSGIPDRFSGSSSGNTHTLTISGARAEDEADYYCASADSSAIPVFGGGT |
| | | | HLTVLGGGSSRSSSSGGGGSGGGGEVQLVESGGDLVKPGGSLRLSCVASG |
| | | | FNFGNYAVSWVRQAPGKGLQWVAGISNDGTITGYTDVVEGRFIISRDNAK |
| | | | NTLYLQMNSLRAEDMAIYYCVAGGFLDYWGQGTLVTVSS |
| 39 | scFv 39 | DNA | CAGTCTGTGCTGACTCAGCCGCCCTCAGTGTCGGGGTCCCCTGGCCAG |
| | | | AGGGTCACCATCTCCTGCTCTGGAACCACGAAGAACATCGGTATAGTC |
| | | | GGTGCGAGTTGGTACCAACAACTCCCAGGAAAGGCCCCTAAACTCCTC |
| | | | GTTCACAGTGATGGGTTTCGACCGTCAGGGGTCCCTGACCGGTTTTCC |
| | | | GCCTCCAACTCTGGCGACTCAGACATTCTGACCATCACTGGGCTTCAG |
| | | | GCTGAGGACGAGGCTGATTATTACTGTCAGTCCTTTGATACCACGCTTG |
| | | | ATGCTCACGTCTTCGGCTCAGGGACCCAGCTGACCGTCCTTGGCGGTG |
| | | | GTTCCTCTAGATCTTCCTCCTCTGGTGGCGGTGGCTCGGGCGGTGGTG |
| | | | GGGAGGTGCCACTGGTGGAATCTGGGGGAGACCTGGTGAAGCCTGGG |
| | | | GGGTCCTTGAGACTGTCCTGTGTGGCCTCTGGATTCACCTTTAGTAGTT |
| | | | ATGGCATGAGCTGGGTCCGTCAGTCTCCAGGGAAGGGGCTGCAGTGG |
| | | | GTCGCAGGTATTACCTATGATGGAAGTAATACATACTACACTGACACTG |
| | | | TGAGGGGCCGATTCACCATCTCCAGAGACAACGCCAGGAACACAGTG |
| | | | TATCTGCAGATGAACAGCCTGAGAGCCGAGGACACAGCTGTGTATTAC |
| | | | TGTGTGGCCAGCCCCCTCTACGGAACTTATGGTTTCGACTACTGGGGC |
| | | | CATGGCACCTCAGTCTTCGTGTCCTCA |
| 82 | scFv 39 | AA | QSVLTQPPSVSGSPGQRVTISCSGTTKNIGIVGASWYQQLPGKAPKLLVHS |
| | | | DGFRPSGVPDRFSASNSGDSDILTITGLQAEDEADYYCQSFDTTLDAHVF |
| | | | GSGTQLTVLGGGSSRSSSSGGGGSGGGGEVPLVESGGDLVKPGGSLRLSC |
| | | | VASGFTFSSYGMSWVRQSPGKGLQWVAGITYDGSNTYYTDTVRGRFTIS |
| | | | RDNARNTVYLQMNSLRAEDTAVYYCVASPLYGTYGFDYWGHGTSVFVS |
| | | | S |
| 40 | scFv 40 | DNA | CAGTCTGTGCTAACTCAGCCGCCCTCAGTGTCTGGGTCCCTGGGCCAG |
| | | | AAGGTCACCATCTCCTGCACTGGAAGCAGTTCCAGCGTCGGTACTCAT |
| | | | TATGTGGGCTGGTACCAAATACCCCCAGGAATAGGCCCTAGAACCGTC |
| | | | ATCTATAATAATGATTACCGACCTTCAGGGGTCCCCGATCGATTCTCTGG |
| | | | CTCCAAGTCAGGCAATTCAGCCACCCTGACCATCTCTGGGCTCCAGGC |
| | | | TGAGGACGAGGCTGATTATTACTGCTCATCATGGGATGACAGTCTCGGA |
| | | | GCTTACGTGCTCGGCTCAGGGACCCAGCTGACCATCCTCGGTGGTGGT |
| | | | TCCTCTAGATCTTCCTCCTCTGGTGGCGGTGGCTCGGGCGGTGGTGGG |
| | | | GAACTCACACTGCAGGAGTCAGGGCCAGGACTGGTGAAGCCCTCACA |
| | | | GACCCTCTCTCTCACCTGCGCTGTGTCCGGAGGCTCCGTCATCAGAGG |
| | | | TTACTACTGGACCTGGATCCGCCAGCGCCCTGGGAGGGGACTGGAATG |
| | | | GATGGGTTACTGGGCAGGGAGTACAGATTATAACCCGGCATTCCAGGG |
| | | | ACGCATCTTCATCACTGCTGGCACGGCCAAGAATCAGTTCTCCCTGGA |
| | | | GATGACTTCCATGACCACGGACGACACGGCCATATATTACTGTGTCGGA |
| | | | TCCCTTCGGGGCGGTTGGCTCTTTGACAACTGGGGCCAGGGAACCCTA |
| | | | GTCACCGTCTCCTCC |
| 83 | scFv 40 | AA | QSVLTQPPSVSGSLGQKVTISCTGSSSSVGTHYVGWYQIPPGIGPRTVIYN |
| | | | NDYRPSGVPDRFSGSKSGNSATLTISGLQAEDEADYYCSSWDDSLGAYVL |
| | | | GSGTQLTILGGGSSRSSSSGGGGSGGGGELTLQESGPGLVKPSQTLSLTCA |
| | | | VSGGSVIRGYYWTWIRQRPGRGLEWMGYWAGSTDYNPAFQGRIFITAGT |
| | | | AKNQFSLEMTSMTTDDTAIYYCVGSLRGGWLFDNWGQGTLVTVSS |
| 41 | scFv 41 | DNA | CAGCCTGTGCTCACTCAGCCGCCCTCGGTCTCAGTGTCTCTGGGACAG |
| | | | ACAGCAACCATCTCCTGCTCTGGAGAGAGTCTGAGTAAATATTATGCAC |
| | | | AGTGGTTCCAGCAGAAGGCAGGCCAAGCCCCTGTCTTGGTCATATATA |
| | | | AGGACACTGAGCGGCCCTCTGGGATCCCTGACCGATTCTCTGGCTCCA |
| | | | GTTCAGGGAACACACACACCCTGACCATCAGCGGGGCTCGGGCCGAG |
| | | | GACGAGGCTGACTATTACTGCGAGTCAGCAGTCAGTACTGAGACTGTT |
| | | | GTGTTCGGCGGAGGCACCCATCTGACCGTCCTCGGCGGTGGTTCCTCT |
| | | | AGATCTTCCTCCTCTGGTGGCGGTGGCTCGGGCGGTGGTGGGGAGGTG |
| | | | CAGCTGGTGGAGACTGGGGGAGACCTGGTGATGCCTGCAGGGTCCCT |
| | | | GAGACTGTCTTGTGTGGCCTCTGGATTCACCTTCAGTAGTTCTGTCATG |
| | | | AGCTGGGTCCGTCAGGCTCCTGGGAAGGGGCTGGAGTGGGTCGCAGG |
| | | | TATTAATGGTGGCGGAAGTTTTGCGGCCTACGGAGACGCTGTGAAGGG |
| | | | CCGATTCACCATATCCAGAGACAACGCCAAGAACACGCTCTATCTGCA |
| | | | GATGAACAGCCTGAGAGCCGAGGACACGGCCATGTATTACTGTGTGGG |
| | | | GGGCGGTTATATGGACTTCTGGGGTCCTGGCACCTCACTCTTCGTGTCC |
| | | | TCA |
| 84 | scFv 41 | AA | QPVLTQPPSVSVSLGQTATISCSGESLSKYYAQWFQQKAGQAPVLVIYKDT |
| | | | ERPSGIPDRFSGSSSGNTHTLTISGARAEDEADYYCESAVSTETVVFGGGT |
| | | | HLTVLGGGSSRSSSSGGGGSGGGGEVQLVETGGDLVMPAGSLRLSCVASG |
| | | | FTFSSSVMSWVRQAPGKGLEWVAGINGGGSFAAYGDAVKGRFTISRDNA |
| | | | KNTLYLQMNSLRAEDTAMYYCVGGGYMDFWGPGTSLFVSS |
| 42 | scFv 42 | DNA | CAGTCTGTGCTGACTCAGCCGACCTCAGTGTCGGGGTCCCTTGGCCAG |
| | | | AGGGTCACCATTTCCTGCTCTGGAAGCACGAACGACATCGGTATTATTG |
| | | | GTGCGGCCTGGTACCAACAGCTCCCAGGGAAGGCCCCTAAACTCCTCC |
| | | | TGTACAGTAATAAGAATCGACCGTCAGGAGTCCCTGACCGATTTTCCG |
| | | | GCTCCAACTCTGGCATCTCAGCCACCCTGACCATCACTGGGCTTCAGG |
| | | | CTGAGGACGAGGCTGATTATTACTGTCAGTCTGTTGATCCCGCGCTTCA |
| | | | TACTTACGTGTTTGGCTCAGGGACCCAGCTGACCATCCTCGGCGGTGG |
| | | | TTCCTCTAGATCTTCCTCCTCTGGTGGCGGTGGCTCGGGCGGTGGTGG |
| | | | GGAGGTGCAGCTGGTGGAGATTGGGGGAGACCTTGTGAAACCTGAGG |
| | | | GGTCCCTGAGACTCTCCTGTGTGGTCTCTGGCTTCACCTTCAGTGGCTA |
| | | | CGACATGAACTGGGTCCGCCAGGCTCCAGGGAAGGGGCTTCAGTGGG |
| | | | TCGCATACATTAGCAGTGATGGGAGGAGAACAGTTTACACAGACGCTG |
| | | | TGAAGGGCCGATTCACCATCTCCAGAGACAATGCCAAGAACACGCTGT |
| | | | ATCTGCAGATGAACAGCCTGAAATCTGAGGACACAGCCGTATATTACT |
| | | | GTGTGAAGGGGGGTTGGTTGGACTTCTGGGGCCAGGGAACCCTGGTC |
| | | | ACCGTCTCCTCA |
| 85 | scFv 42 | AA | QSVLTQPTSVSGSLGQRVTISCSGSTNDIGIIGAAWYQQLPGKAPKLLLYS |
| | | | NKNRPSGVPDRFSGSNSGISATLTITGLQAEDEADYYCQSVDPALHTYVF |
| | | | GSGTQLTILGGGSSRSSSSGGGGSGGGGEVQLVEIGGDLVKPEGSLRLSCV |
| | | | VSGFTFSGYDMNWVRQAPGKGLQWVAYISSDGRRTVYTDAVKGRFTISR |
| | | | DNAKNTLYLQMNSLKSEDTAVYYCVKGGWLDFWGQGTLVTVSS |
| 43 | scFv 43 | DNA | GATATTGTCATGACACAGAACCCACTGTCCCTGGCCGTCACCCCTGGA |
| | | | GAGCTGGCCACTATCTCCTGCAGGGCCAGTCAGAGTCTCCTGCACAGT |
| | | | GATGGAAAATCCTATTTGAATTGGTACCTGCAGAAGCCAGGCCAGACT |
| | | | CCTCGGCCGCTGATTTATGAGGCTTCCAGGCGTTTCTCTGGGGTCTCAG |
| | | | ACAGGTTCAATGGCAGCGGGTCAGGGACAGATTTCACCCTTAAAATCA |
| | | | GTAGGGTGGAGGCTGAGGATGTTGGAGTTTATTACTGTCAGCAAGGTC |
| | | | TACATTTTCCTCCGACGTTCGGAGCAGGAACCAAGGTGGAGCTCAAAG |
| | | | GTGGTTCCTCTAGATCTTCCTCCTCTGGTGGCGGTGGCTCGGGCGGTG |
| | | | GTGGGGAGGTACAGCTGGTGGAATCTGGGGGAGACCTGGTGAAGCCT |
| | | | GGGGGGTCCCTGAGACTCTCCTGTGCAGCCTCTGGATTCACCTTCACT |
| | | | GACTACGACATGAGTTGGGTCCGCCAGGCTCCTGGGAAGGGGCTGCA |
| | | | GTGGGTCGCAGCTATTAGCTATGATGGAAGTAGCACATACTACACTGAC |
| | | | GCTGTGAAGGGCCGATTCACCATCTCCAGAGACAACGCCAGGAACAC |
| | | | ACTGTATCTGCAGATGAGCAGCCTGAGAGCCGAGGACACGGCCATGTA |
| | | | TTACTGTGTAGTCTCGAATATGAACCTCTGGGGCCATGGCACCTCAGTC |
| | | | TTCGTGTCCTCT |
| 86 | scFv 43 | AA | DIVMTQNPLSLAVTPGELATISCRASQSLLHSDGKSYLNWYLQKPGQTPR |
| | | | PLIYEASRRFSGVSDRFNGSGSGTDFTLKISRVEAEDVGVYYCQQGLHFPP |
| | | | TFGAGTKVELKGGSSRSSSSGGGGSGGGGEVQLVESGGDLVKPGGSLRLS |
| | | | CAASGFTFTDYDMSWVRQAPGKGLQWVAAISYDGSSTYYTDAVKGRFTI |
| | | | SRDNARNTLYLQMSSLRAEDTAMYYCVVSNMNLWGHGTSVFVSS |

In some embodiments, the anti-CD19 amino acid sequence encoded by the first nucleic acid sequence encodes a polypeptide comprising one or more anti-CD19 CDRs capable of binding or specifically binding to CD19. As used herein, “specifically binds” means that the binding domain preferentially binds the corresponding target over other proteins. In some embodiments, “specifically binds” means that the binding domains have a higher affinity for the target than for other proteins.

In some embodiments, the one or more anti-CD19 CDR amino acid sequence encoded by the first nucleic acid sequence are selected from the CDR amino acid sequences in Table 1B. In some embodiments, the one or more anti-CD19 CDR amino acid sequence encoded by the first nucleic acid sequence are selected from one or more CDR amino acid sequence of SEQ ID NOS: 87-247 in Table 1B. In some embodiments, the one or more anti-CD19 CDR amino acid sequence encoded by the first nucleic acid sequence are selected from one or more functional variant of one or more CDR amino acid sequences of SEQ ID NOS: 87-247 having at least about 70%, about 75%, about 80%, about 85%, about 90%, about 91%, about 92%, about 93%, about 94%, about 95%, about 96%, about 97%, about 98%, about 99% or more sequence identity to the amino acid sequence(s) of SEQ ID NOS: 87-247. In some embodiments, the one or more anti-CD19 CDR amino acid sequence comprises at least three to six anti-CD19 CDR amino acid sequences. In some embodiments, the at least three to six anti-CD19 CDR amino acid sequence comprise, for a VL, a set of VL 1, 2, and 3 sequences from Table 1B, or a functional variant of the each having at least about 70%, about 75%, about 80%, about 85%, about 90%, about 91%, about 92%, about 93%, about 94%, about 95%, about 96%, about 97%, about 98%, about 99% or more sequence identity to the respective amino acid sequences of the set. In some embodiments, the at least three to six anti-CD19 CDR amino acid sequence comprise, for a VH, a set of VH 1, 2, and 3 sequences from Table 1B, or a functional variant of the each having at least about 70%, about 75%, about 80%, about 85%, about 90%, about 91%, about 92%, about 93%, about 94%, about 95%, about 96%, about 97%, about 98%, about 99% or more sequence identity to the respective amino acid sequences of the set.

In some embodiments, the anti-CD19 amino acid sequence encoded by the first nucleic acid sequence encodes a polypeptide comprising an HCDR1, HCDR2, and HCDR3 of SEQ ID NOs: 163, 164, and 123, respectively.

In some embodiments, the anti-CD19 amino acid sequence encoded by the first nucleic acid sequence encodes a polypeptide comprising an LCDR1, LCDR2, and LCDR3 of SEQ ID NOs: 124, GYN, and 165, respectively.

The skilled artisan recognizes a CDR can be at about amino acid residues 24-34 (L1), 50-56 (L2) and 89-97 (L3) in the light chain variable domain and at about amino acid residues 31-35 (H1), 50-65 (H2) and 95-102 (H3) in the heavy chain variable domain as defined by Kabat et al. (Kabat et al. Sequences of Proteins of Immunological Interest, 5th Ed. Public Health Service, National Institutes of Health, Bethesda, Md. (1991)). Alternative definitions of the CDRs are also recognized in the art, for example, according to the scheme of Chothia (Chothia and Lesk (1987) J. Mol. Biol. 196: 901-917) in which case the CDRs are found at about amino acid residues 26-32 (L1), 50-52 (L2) and 91-96 (L3) in the light chain, and at about amino acid residues 26-32 (H1), 53-55 (H2) and 96-101 (H3) in the heavy chain. In some embodiments, the one or more anti-CD19 CDR are selected from an anti-CD19 VL or VH herein at the foregoing positions. In some embodiments, the one or more anti-CD19 CDR comprises from about three to about six anti-CD19 CDR selected from an anti-CD19 VL or VH herein at the foregoing positions.

TABLE 1B
Exemplary CDRs of Anti-CD19 scFvs

| SEQ | | | |
|---|---|---|---|
| ID | | | |
| NO: | Name: | Type: | Sequence: |
| 87 | scFv_01_H CDR1 | AA | GFTFSNHE |
| 88 | scFv_01_H CDR2 | AA | IYDSGSRT |
| 89 | scFv_01_H CDR3 | AA | AGGNSRGWTEFGMDY |
| 90 | scFv_01_L CDR1 | AA | NTNIGSPYD |
| | scFv_01_L CDR2 | AA | GNS |
| 91 | scFv_01_L CDR3 | AA | QSYDDSLDGRVV |
| 92 | scFv_02_H CDR1 | AA | GFSFSSYD |
| 93 | scFv_02_H CDR2 | AA | TNFDGSRT |
| 94 | scFv_02_H CDR3 | AA | ARGMGSWGASTFGY |
| 95 | scFv_02_L CDR1 | AA | RSNLGGNY |
| | scFv_02_L CDR2 | AA | AND |
| 96 | scFv_02_L CDR3 | AA | SSWDDDLGSHV |
| 97 | scFv_03_H CDR1 | AA | GFTFSNYT |
| 98 | scFv_03_H CDR2 | AA | INYDGSIT |
| 99 | scFv_03_H CDR3 | AA | VKETFMIPTE |
| 100 | scFv_03_L CDR1 | AA | HTNIGSGFD |
| | scFv_03_L CDR2 | AA | ANI |
| 101 | scFv_03_L CDR3 | AA | QSYDDNFDGHV |
| 102 | scFv_04_H CDR1 | AA | GFDFSRYG |
| 103 | scFv_04_H CDR2 | AA | ISYRGTTF |
| 104 | scFv_04_H CDR3 | AA | NSANWYVSPFDH |
| 100 | scFv_04_L CDR1 | AA | HTNIGSGFD |
| | scFv_04_L CDR2 | AA | ANI |
| 105 | scFv_04_L CDR3 | AA | QSYDDNFDYV |
| 106 | scFv_05_H CDR1 | AA | GFPFSGYS |
| 107 | scFv_05_H CDR2 | AA | INSDGSHT |
| 108 | scFv_05_H CDR3 | AA | GSHSWGNFHY |
| 109 | scFv_05_L CDR1 | AA | TNTIGVVG |
| | scFv_05_L CDR2 | AA | SDG |
| 110 | scFv_05_L CDR3 | AA | QSFDSTHNAIV |
| 111 | scFv_06_H CDR1 | AA | GFTFSDNY |
| 112 | scFv_06_H CDR2 | AA | ISTDGRGT |
| 113 | scFv_06_H CDR3 | AA | ARRSGIWGFDY |
| 114 | scFv_06_L CDR1 | AA | NTNIGSGYS |
| | scFv_06_L CDR2 | AA | GSS |
| 115 | scFv_06_L CDR3 | AA | QSYDDSLDGHAV |
| 116 | scFv_07_H CDR1 | AA | GFTFSNYG |
| 117 | scFv_07_H CDR2 | AA | INSGGSTT |
| 118 | scFv_07_H CDR3 | AA | ARVDYSSSWLVDY |
| 119 | scFv_07_L CDR1 | AA | NTNIGSGYD |
| | scFv_07_L CDR2 | AA | GNT |
| 120 | scFv_07_L CDR3 | AA | QSYDDNFDGHHV |
| 121 | scFv_08_H CDR1 | AA | GFTFSSYG |
| 122 | scFv_08_H CDR2 | AA | ISYGGGTY |
| 123 | scFv_08_H CDR3 | AA | SQWGDWGHFEY |
| 124 | scFv_08_L CDR1 | AA | RSNIGANY |
| | scFv_08_L CDR2 | AA | GYN |
| 125 | scFv_08_L CDR3 | AA | SSWDDSLRGTV |
| 126 | scFv_09_H CDR1 | AA | GFTFSSDS |
| 117 | scFv_09_H CDR2 | AA | INSGGSTT |
| 127 | scFv_09_H CDR3 | AA | AKDSGFGSWYLES |
| 128 | scFv_09_L CDR1 | AA | KTNIGSGYD |
| | scFv_09_L CDR2 | AA | GNS |
| 129 | scFv_09_L CDR3 | AA | QSFDDNVDGYV |
| 130 | scFv_10_H CDR1 | AA | GLTFNRNY |
| 131 | scFv_10_H CDR2 | AA | INPDGSTT |
| 132 | scFv_10_H CDR3 | AA | ARSSWGSFDY |
| 100 | scFv_10_L CDR1 | AA | HTNIGSGFD |
| | scFv_10_L CDR2 | AA | ANI |
| 133 | scFv_10_L CDR3 | AA | QSFDDDLDGPV |
| 134 | scFv_11_H CDR1 | AA | GFTFSDYS |
| 135 | scFv_11_H CDR2 | AA | IMTSGSTY |
| 136 | scFv_11_H CDR3 | AA | HDYWFDF |
| 100 | scFv_11_L CDR1 | AA | HTNIGSGFD |
| | scFv_11_L CDR2 | AA | ANI |
| 105 | scFv_11_L CDR3 | AA | QSYDDNFDYV |
| 137 | scFv_12_H CDR1 | AA | GFTFSSYD |
| 138 | scFv_12_H CDR2 | AA | ISYDGSST |
| 139 | scFv_12_H CDR3 | AA | AKDSGSWSHFYGLDT |
| 140 | scFv_12_L CDR1 | AA | NIGRKS |
| | scFv_12_L CDR2 | AA | ADS |
| 141 | scFv_12_L CDR3 | AA | QIWDRNINV |
| 142 | scFv_13_H CDR1 | AA | GFTFSRYY |
| 143 | scFv_13_H CDR2 | AA | ISSDGRRT |
| 144 | scFv_13_H CDR3 | AA | VREDYFDFDY |
| 100 | scFv_13_L CDR1 | AA | HTNIGSGFD |
| | scFv_13_L CDR2 | AA | ANI |
| 105 | scFv_13_L CDR3 | AA | QSYDDNFDYV |
| 145 | scFv_14_H CDR1 | AA | GFAFSGKT |
| 146 | scFv_14_H CDR2 | AA | IQHDGSRT |
| 147 | scFv_14_H CDR3 | AA | ADHPWYGPEY |
| 148 | scFv_14_L CDR1 | AA | HTNIGSGYD |
| | scFv_14_L CDR2 | AA | GNS |
| 149 | scFv_14_L CDR3 | AA | QSYDDNLNDPYV |
| 150 | scFv_15_H CDR1 | AA | GFTFNKYH |
| 151 | scFv_15_H CDR2 | AA | ITSDGSAT |
| 152 | scFv_15_H CDR3 | AA | ARDQSSWGTGNFDY |
| 153 | scFv_15_L CDR1 | AA | DTNIGNHYD |
| | scFv_15_L CDR2 | AA | GNS |
| 154 | scFv_15_L CDR3 | AA | QSYDDDLDGHIV |
| 155 | scFv_16_H CDR1 | AA | GFTLSSYW |
| 156 | scFv_16_H CDR2 | AA | ISGSGNTI |
| 157 | scFv_16_H CDR3 | AA | ATEYYWNIDN |
| 158 | scFv_16_L CDR1 | AA | SSNVGYGNY |
| | scFv_16_L CDR2 | AA | GSH |
| 159 | scFv_16_L CDR3 | AA | SSYDSTLSGYV |
| 121 | scFv_17_H CDR1 | AA | GFTFSSYG |
| 160 | scFv_17_H CDR2 | AA | INGGGSST |
| 161 | scFv_17_H CDR3 | AA | SNYYGIYWDSDY |
| 128 | scFv_17_L CDR1 | AA | KTNIGSGYD |
| | scFv_17_L CDR2 | AA | GNS |
| 162 | scFv_17_L CDR3 | AA | QSFDDNVDGYV |
| 163 | scFv_18_H CDR1 | AA | GFTLSNYG |
| 164 | scFv_18_H CDR2 | AA | IRSGGNTY |
| 123 | scFv_18_H CDR3 | AA | SQWGDWGHFEY |
| 124 | scFv_18_L CDR1 | AA | RSNIGANY |
| | scFv_18_L CDR2 | AA | GYN |
| 165 | scFv_18_L CDR3 | AA | STWDSSLNAYV |
| 106 | scFv_19_H CDR1 | AA | GFPFSGYS |
| 107 | scFv_19_H CDR2 | AA | INSDGSHT |
| 108 | scFv_19_H CDR3 | AA | GSHSWGNFHY |
| 166 | scFv_19_L CDR1 | AA | TNNIGIVG |
| | scFv_19_L CDR2 | AA | SDG |
| 167 | scFv_19_L CDR3 | AA | QSVDATHGTIL |
| 168 | scFv_20_H CDR1 | AA | GFTFSSYA |
| 169 | scFv_20_H CDR2 | AA | INSAGSTT |
| 170 | scFv_20_H CDR3 | AA | GRYYYGLDY |
| 171 | scFv_20_L CDR1 | AA | SLSKYY |
| | scFv_20_L CDR2 | AA | KDT |
| 172 | scFv_20_L CDR3 | AA | ESEVSPDTIV |
| 173 | scFv_21_H CDR1 | AA | GFTFSSFW |
| 174 | scFv_21_H CDR2 | AA | INSGGSIT |
| 175 | scFv_21_H CDR3 | AA | AKPDIYGSFDH |
| 171 | scFv_21_L CDR1 | AA | SLSKYY |
| | scFv_21_L CDR2 | AA | KDT |
| 176 | scFv_21_L CDR3 | AA | ESAVSSDTAV |
| 177 | scFv_22_H CDR1 | AA | GDSVTSSHF |
| 178 | scFv_22_H CDR2 | AA | WSDIT |
| 179 | scFv_22_H CDR3 | AA | ARYNGYVDYLDV |
| 180 | scFv_22_L CDR1 | AA | SSNVGYGDY |
| | scFv_22_L CDR2 | AA | SSS |
| 181 | scFv_22_L CDR3 | AA | SSYDTSLNGFV |
| 182 | scFv_23_H CDR1 | AA | GGSVTTSDY |
| 183 | scFv_23_H CDR2 | AA | WKGRT |
| 184 | scFv_23_H CDR3 | AA | AHWGSGNLDY |
| 185 | scFv_23_L CDR1 | AA | QSLLHSNGNTY |
| | scFv_23_L CDR2 | AA | LVS |
| 186 | scFv_23_L CDR3 | AA | GQGKQDPIT |
| 187 | scFv_24_H CDR1 | AA | GFTLSSNY |
| 188 | scFv_24_H CDR2 | AA | ISTDGSRT |
| 189 | scFv_24_H CDR3 | AA | AKNEYLAPTY |
| 100 | scFv_24_L CDR1 | AA | HTNIGSGFD |
| | scFv_24_L CDR2 | AA | ANI |
| 190 | scFv_24_L CDR3 | AA | QSYDDNVDGYV |
| 191 | scFv_25_H CDR1 | AA | GFTFSSLS |
| 192 | scFv_25_H CDR2 | AA | ISPDGTIT |
| 193 | scFv_25_H CDR3 | AA | AKSRYTDTYPFDY |
| 185 | scFv_25_L CDR1 | AA | QSLLHSNGNTY |
| | scFv_25_L CDR2 | AA | QVS |
| 194 | scFv_25_L CDR3 | AA | GQGIQYPPT |
| 195 | scFv_26_H CDR1 | AA | GFTFSSYW |
| 196 | scFv_26_H CDR2 | AA | ISDSGSST |
| 197 | scFv_26_H CDR3 | AA | ARGLGMDY |
| 171 | scFv_26_L CDR1 | AA | SLSKYY |
| | scFv_26_L CDR2 | AA | KDT |
| 198 | scFv_26_L CDR3 | AA | ESLVDSDAYM |
| 195 | scFv_27_H CDR1 | AA | GFTFSSYW |
| 196 | scFv_27_H CDR2 | AA | ISDSGSST |
| 197 | scFv_27_H CDR3 | AA | ARGLGMDY |
| 171 | scFv_27_L CDR1 | AA | SLSKYY |
| | scFv_27_L CDR2 | AA | KDT |
| 199 | scFv_27_L CDR3 | AA | HSYDENLDGYV |
| 195 | scFv_28_H CDR1 | AA | GFTFSSYW |
| 196 | scFv_28_H CDR2 | AA | ISDSGSST |
| 197 | scFv_28_H CDR3 | AA | ARGLGMDY |
| 200 | scFv_28_L CDR1 | AA | SLNKYY |
| | scFv_28_L CDR2 | AA | RDT |
| 201 | scFv_28_L CDR3 | AA | ESLVNDNTYV |
| 195 | scFv_29_H CDR1 | AA | GFTFSSYW |
| 196 | scFv_29_H CDR2 | AA | ISDSGSST |
| 197 | scFv_29_H CDR3 | AA | ARGLGMDY |
| 200 | scFv_29_L CDR1 | AA | SLNKYY |
| | scFv_29_L CDR2 | AA | KDT |
| 202 | scFv_29_L CDR3 | AA | ESVDSTDTYV |
| 195 | scFv_30_H CDR1 | AA | GFTFSSYW |
| 196 | scFv_30_H CDR2 | AA | ISDSGSST |
| 197 | scFv_30_H CDR3 | AA | ARGLGMDY |
| 171 | scFv_30_L CDR1 | AA | SLSKYY |
| | scFv_30_L CDR2 | AA | KDT |
| 190 | scFv_30_L CDR3 | AA | QSYDDNVDGYV |
| 195 | scFv_31_H CDR1 | AA | GFTFSSYW |
| 196 | scFv_31_H CDR2 | AA | ISDSGSST |
| 197 | scFv_31_H CDR3 | AA | ARGLGMDY |
| 203 | scFv_31_L CDR1 | AA | SLTKYY |
| | scFv_31_L CDR2 | AA | RDT |
| 204 | scFv_31_L CDR3 | AA | ESMDGREAYV |
| 195 | scFv_32_H CDR1 | AA | GFTFSSYW |
| 196 | scFv_32_H CDR2 | AA | ISDSGSST |
| 197 | scFv_32_H CDR3 | AA | ARGLGMDY |
| 171 | scFv_32_L CDR1 | AA | SLSKYY |
| | scFv_32_L CDR2 | AA | KDT |
| 205 | scFv_32_L CDR3 | AA | QSYDDNLDGYV |
| 206 | scFv_33_H CDR1 | AA | GFTFSHYD |
| 207 | scFv_33_H CDR2 | AA | IAYDGSNT |
| 208 | scFv_33_H CDR3 | AA | ARGLVY |
| 209 | scFv_33_L CDR1 | AA | QSLLHSDGNTY |
| | scFv_33_L CDR2 | AA | SVS |
| 210 | scFv_33_L CDR3 | AA | GQGTHLPFT |
| 211 | scFv_34_H CDR1 | AA | GLTFSDNY |
| 112 | scFv_34_H CDR2 | AA | ISTDGRGT |
| 113 | scFv_34_H CDR3 | AA | ARRSGIWGFDY |
| 114 | scFv_34_L CDR1 | AA | NTNIGSGYS |
| | scFv_34_L CDR2 | AA | GSS |
| 115 | scFv_34_L CDR3 | AA | QSYDDSLDGHAV |
| 168 | scFv_35_H CDR1 | AA | GFTFSSYA |
| 117 | scFv_35_H CDR2 | AA | INSGGSTT |
| 212 | scFv_35_H CDR3 | AA | ARWWDFEY |
| 213 | scFv_35_L CDR1 | AA | SLSEYY |
| | scFv_35_L CDR2 | AA | KDT |
| 214 | scFv_35_L CDR3 | AA | ESAVNSDGIV |
| 121 | scFv_36_H CDR1 | AA | GFTFSSYG |
| 215 | scFv_36_H CDR2 | AA | ISDTGSTY |
| 216 | scFv_36_H CDR3 | AA | KDLGVWGMDN |
| 166 | scFv_36_L CDR1 | AA | TNNIGIVG |
| | scFv_36_L CDR2 | AA | SDG |
| 217 | scFv_36_L CDR3 | AA | QSFDTTLDAYV |
| 218 | scFv_37_H CDR1 | AA | GFTLRDYS |
| 219 | scFv_37_H CDR2 | AA | ISTGGSTT |
| 220 | scFv_37_H CDR3 | AA | TTTALGY |
| 221 | scFv_37_L CDR1 | AA | SLNDYY |
| | scFv_37_L CDR2 | AA | RDT |
| 222 | scFv_37_L CDR3 | AA | ESEVSAGGAV |
| 223 | scFv_38_H CDR1 | AA | GFNFGNYA |
| 224 | scFv_38_H CDR2 | AA | ISNDGTIT |
| 225 | scFv_38_H CDR3 | AA | VAGGFLDY |
| 213 | scFv_38_L CDR1 | AA | SLSEYY |
| | scFv_38_L CDR2 | AA | KDT |
| 226 | scFv_38_L CDR3 | AA | ASADSSAIPV |
| 121 | scFv_39_H CDR1 | AA | GFTFSSYG |
| 227 | scFv_39_H CDR2 | AA | ITYDGSNT |
| 228 | scFv_39_H CDR3 | AA | VASPLYGTYGFDY |
| 229 | scFv_39_L CDR1 | AA | TKNIGIVG |
| | scFv_39_L CDR2 | AA | SDG |
| 230 | scFv_39_L CDR3 | AA | QSFDTTLDAHV |
| 231 | scFv_40_H CDR1 | AA | GGSVIRGYY |
| 232 | scFv_40_H CDR2 | AA | WAGST |
| 233 | scFv_40_H CDR3 | AA | VGSLRGGWLFDN |
| 234 | scFv_40_L CDR1 | AA | SSSVGTHY |
| | scFv_40_L CDR2 | AA | NND |
| 235 | scFv_40_L CDR3 | AA | SSWDDSLGAYV |
| 236 | scFv_41_H CDR1 | AA | GFTFSSSV |
| 237 | scFv_41_H CDR2 | AA | INGGGSFA |
| 238 | scFv_41_H CDR3 | AA | VGGGYMDF |
| 171 | scFv_41_L CDR1 | AA | SLSKYY |
| | scFv_41_L CDR2 | AA | KDT |
| 239 | scFv_41_L CDR3 | AA | ESAVSTETVV |
| 240 | scFv_42_H CDR1 | AA | GFTFSGYD |
| 143 | scFv_42_H CDR2 | AA | ISSDGRRT |
| 241 | scFv_42_H CDR3 | AA | VKGGWLDF |
| 242 | scFv_42_L CDR1 | AA | TNDIGIIG |
| | scFv_42_L CDR2 | AA | SNK |
| 243 | scFv_42_L CDR3 | AA | QSVDPALHTYV |
| 244 | scFv_43_H CDR1 | AA | GFTFTDYD |
| 138 | scFv_43_H CDR2 | AA | ISYDGSST |
| 245 | scFv_43_H CDR3 | AA | VVSNMNL |
| 246 | scFv_43_L CDR1 | AA | QSLLHSDGKSY |
| | scFv_43_L CDR2 | AA | EAS |
| 247 | scFv_43_L CDR3 | AA | QQGLHFPPT |

In some embodiments, the anti-CD19 amino acid sequence encoded by a first nucleic acid sequence comprises an anti-CD19 light chain (VL). In some embodiments, the anti-CD19 light chain comprises the amino acid sequence of one of SEQ ID NOS: 271-313. See Table 1C. In some embodiments, the anti-CD19 light chain comprise the amino acid sequence of a functional variant of one of SEQ ID NOS: 271-313 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 271-313.

In some embodiments, the anti-CD19 amino acid sequence encoded by a first nucleic acid sequence comprises an anti-CD19 light chain (VL) having at least about 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 288. In some embodiments, the anti-CD19 amino acid sequence encoded by a first nucleic acid sequence comprises an anti-CD19 light chain (VL) of SEQ ID NO: 288.

In some embodiments, the anti-CD19 amino acid sequence encoded by a first nucleic acid sequence comprises an anti-CD19 heavy chain (VH). In some embodiments, the anti-CD19 heavy chain comprises the amino acid sequence of one of SEQ ID NOS: 314-356. See Table 1C. In some embodiments, the anti-CD19 heavy chain comprises the amino acid sequence of a functional variant of one of SEQ ID NOS: 314-356 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 314-356.

In some embodiments, the anti-CD19 amino acid sequence encoded by a first nucleic acid sequence comprises an anti-CD19 heavy chain (VH) having at least about 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 331. In some embodiments, the anti-CD19 amino acid sequence encoded by a first nucleic acid sequence comprises an anti-CD19 heavy chain (VH) of SEQ ID NO: 331.

In some embodiments, the anti-CD19 antigen binding domain comprises a light chain and a heavy chain. In some embodiments, the light chain comprises the amino acid sequence of one of SEQ ID NOS: 271-313. See Table 1C. In some embodiments, the light chain comprise the amino acid sequence of a functional variant of one of SEQ ID NOS: 271-313 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 271-313. In some embodiments, the heavy chain comprises the amino acid sequence of one of SEQ ID NOS: 314-356. See Table 1C. In some embodiments, the heavy chain comprises the amino acid sequence of a functional variant of one of SEQ ID NOS: 314-356 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 314-356. In some embodiments, the anti-CD20 binding domain comprises a VH of SEQ ID NO: 331 and a VL of SEQ ID NO: 288.

In some embodiments, the light chain precedes, that is, it is N-terminal to, the heavy chain. In some embodiments, the heavy chain precedes, that is, it is N-terminal to, the light chain. In some embodiments, an LH linker interposes between the light chain and the heavy chain. In some embodiments, the LH linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in length. In some embodiments, the linker is 19 amino acids in length. In some embodiments, the linker comprises the amino acid sequence GGGSSRSSSSGGGGSGGGG (SEQ ID NO: 413) or a function variant of the amino acid sequence GGGSSRSSSSGGGGSGGGG (SEQ ID NO: 413) having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 413. In some embodiments, the LH linker is encoded by a nucleic acid sequence comprising the sequence of one of SEQ ID NOS: 821-829, 831, or 833, or a functional variant of one of SEQ ID NOS: 821-829, 831, or 833 having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 821-829, 831, or 833. In some embodiments, the LH linker comprises an amino acid sequence comprising the sequence of SEQ ID NO: 830 or 832, or a function variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 830 or 832.

TABLE 1C
Exemplary Amino Acid Sequences of Anti-CD19 Light and Heavy Chains

| SEQ | | |
|---|---|---|
| ID | | |
| NO: | Name: | Sequence: |
| 271 | scFv_01 VL | QAVLNQPASVSAALGQRVTISCNTNIGSPYDVQWYQQLPGKSPKTIIYGNSNRP |
| | | SGVPVRFSGSKSGSTATLTIAGIQAEDEADYYCQSYDDSLDGRVVFGGGTQLTV |
| | | L |
| 272 | scFv_02 VL | SYVLTQSPSLSGSLGQKVTISCTGSRSNLGGNYVGWYQQVPGMAPRNVIYAND |
| | | FRPSRIPARFSASKSGTSATLTISGLQAEDEADYFCSSWDDDLGSHVFGSGTQLTI |
| | | L |
| 273 | scFv_03 VL | QSVLNQPPSVSGALGQTVTISCSGSHTNIGSGFDVQWYQQLPGKSPQTLVYANI |
| | | NRPSGVPARFSGSKSGSTATLTITGVQAEDEADYYCQSYDDNFDGHVFGSGTQ |
| | | LTVL |
| 274 | scFv_04 VL | QSVLNQPPSVSGALGQTVTISCSGSHTNIGSGFDVQWYQQLPGKSPQTIIYANIN |
| | | RPSGVPARFSGSKSGSTATLTITGVQAEDEADYYCQSYDDNFDYVFGSGTQLTV |
| | | L |
| 275 | scFv_05 VL | QPVLTQPPSVSGSLGQRVTISCSGSTNTIGVVGANWYQQLPGKSPKLLVYSDGV |
| | | RPSGVPDRFSGSRSANSDTLTITGLQAEDEADYYCQSFDSTHNAIVFGGGTHLT |
| | | VL |
| 276 | scFv_06 VL | QPVLTQPPSVSGSLGQRVTISCTGTNTNIGSGYSVQWYQQLPGESPKPIIYGSSN |
| | | RPSGVPARFSGSKSGSTGTLTITGIQAEDEADYHCQSYDDSLDGHAVFGGGTQL |
| | | TIL |
| 277 | scFv_07 VL | SYELTQPPSMSAALGQRVTISCTGSNTNIGSGYDVQWYQQVPGKSPTPLIYGNT |
| | | NRPSGVPVRFSGSKSGNTATLTITGIQAEDEADYYCQSYDDNFDGHHVFGSGTQ |
| | | LTVL |
| 278 | scFv_08 VL | QSVLTQPASVSGSLGQKVTISCTGSRSNIGANYVGWYQQVPGIGPRTVIFGYNY |
| | | RPSGVPDRESSSKSGNSATLTISGLQAEDEAEYYCSSWDDSLRGTVFGSGTQLT |
| | | VL |
| 279 | scFv_09 VL | QPVLTQPPSVSAALGQRVTLSCTGSKTNIGSGYDVQWYQQFPGKSPKTIIYGNS |
| | | NRFSGVPARFSGSKSGSIATLTITGIQAEDEADYYCQSFDDNVDGYVFGSGTQLT |
| | | IL |
| 280 | scFv_10 VL | QAVLNQPASVSGALGQTVTISCSGSHTNIGSGFDVQWYQQLPGKSPQTIIYANIN |
| | | RPSGVPARFSGSKSGSTATLTITGVQAEDEADYHCQSFDDDLDGPVFGGGTHLT |
| | | VL |
| 281 | scFv_11 VL | QPVLTQPPSVSGALGQTVTISCSGSHTNIGSGFDVQWYQQLPGKSPQTLVYANI |
| | | NRPSGVPARFSGSKSGSTATLTITGVQAEDEADYYCQSYDDNFDYVFGSGTQLT |
| | | VL |
| 282 | scFv_12 VL | SYELTQPPSATVTLRQTARLTCGGENIGRKSVQWYQQKPGQSPLLIIYADSSRPS |
| | | GIPERFSGANSGNTASLTISGALAEDEADYYCQIWDRNINVFGSGTQLTVL |
| 283 | scFv_13 VL | QPVLTQPPSVSGALGQTVTISCSGSHTNIGSGFDVQWYQQLPGKSPQTLVYANI |
| | | NRPSGVPARFSGSKSGSTATLTITGVQAEDEADYYCQSYDDNFDYVFGSGTQLT |
| | | VL |
| 284 | scFv_14 VL | QSVLNQPPSVSAALGQRITISCTGSHTNIGSGYDVQWYQQLPGKSPRTIIYGNSD |
| | | RPSGVPVRFSGSKSGNTATLTITGIQAEDEADYYCQSYDDNLNDPYVFGSGTQL |
| | | TVL |
| 285 | scFv_15 VL | SYELTQPPSVSAALGQGVTISCIGSDTNIGNHYDVQWYQQLPGKSPRTIIYGNSD |
| | | RPSGVPGRFSGSKSGNTATLTITGIQAEDEADYHCQSYDDDLDGHIVFGGGTHL |
| | | TVL |
| 286 | scFv_16 VL | QSVLTQPTSVSGSLGQRVTISCTGSSSNVGYGNYVGWYQQLPGTGPRTLIYGSH |
| | | YRPSGVPDRFSGSSSGSSATLTISGLQAEDEADYYCSSYDSTLSGYVFGSGTQLT |
| | | IL |
| 287 | scFv_17 VL | QPVLTQPPSVSAALGQRVTISCTGSKTNIGSGYDVQWYQQLPGKSPKTIIYGNS |
| | | NRPSGVPARFSGSKSGSIATLTITGIQAEDEADYYCQSFDDNVDGYVFGSGTQLT |
| | | IL |
| 288 | scFv_18 VL | QSALTQTASVSGSLGQKVTISCTGSRSNIGANYVGWYQQVPGIGPRTVIFGYNY |
| | | RPSGVPDRFSSSKSGNSATLTISGLQAEDEAEYYCSTWDSSLNAYVFGSGTQLT |
| | | VL |
| 289 | scFv_19 VL | QAVLTQPASVSGPLGQRVTISCSGSTNNIGIVGAAWYQQFPGKAPKLLVYSDGS |
| | | RPSGVPDRFSGSNSGNSATLTISGLQAEDEADYYCQSVDATHGTILFGGGTQLT |
| | | VL |
| 290 | scFv_20 VL | QSVLNQPPSVSVSLGQTATISCSGESLSKYYAQWFQQKAGQVPVLVIYKDTERP |
| | | SGIPDRFSGSSSGNTHTLTISGARAEDEADYYCESEVSPDTIVFGGGTHLTVL |
| 291 | scFv_21 VL | QPVLTQPPSVSVSLGQTATISCSGESLSKYYAQWFQQKAGQAPVLVIYKDTERP |
| | | SGIPDRFSGSSSGNTHTLTISGARAEDEADYYCESAVSSDTAVFGSGTQLTVL |
| 292 | scFv_22 VL | QPVLTQPPSVSGSLGQRVTISCTGSSSNVGYGDYVGWYQQLPGTSPRTLIYSSSS |
| | | RPSGVPDRFSGSRSGSTATLTISGLQAEDEADYHCSSYDTSLNGFVFGSGTQLTI |
| | | L |
| 293 | scFv_23 VL | DIVMTQTPLSLSVSPGEPASISCKASQSLLHSNGNTYLYWFRQKPGQSPQRLIYL |
| | | VSNRDAGVPDRFSGSGSGTDFTLRISRVEADDSGIYYCGQGKQDPITFGEGTHL |
| | | EIK |
| 294 | scFv_24 VL | QSVLNQPPSVSGALGQTVTISCSGSHTNIGSGFDVQWYQQLPGKSPQTLVYANI |
| | | NRPSGVPVRFSGSKSGTTATLTITGIQAEDEADYYCQSYDDNVDGYVFGSGTQL |
| | | TVL |
| 295 | scFv_25 VL | DIVMTQTPLSLSVSPGETASISCKASQSLLHSNGNTYLNWFRQKPGQSPQVLIY |
| | | QVSNRDTGVPDRFSGSGSGTDFTLRISRVEADDAGVYYCGQGIQYPPTFGAGT |
| | | KVELK |
| 296 | scFv_26 VL | SSVLTQPPSVSVSLGQTATISCSGESLSKYYTQWFQQKAGQAPILVIYKDTERPS |
| | | GIPDRFSGSSSGNTHTLTISGARAEDEADYYCESLVDSDAYMFGSGTQLTVL |
| 297 | scFv_27 VL | QPVLTQPPSVSVSLGQTATISCSGESLSKYYAQWFQQKAGQAPVLVIYKDTERP |
| | | SGIPDRFSGSSSGNTHTLTITGIQAEDEGDYHCHSYDENLDGYVFGSGTQLTVL |
| 298 | scFv_28 VL | QSALTQPSSVSVSLGQTATISCSGESLNKYYVQWFQQKAGQVPILVIYRDTERPS |
| | | GIPDRFSGSSSGNTHTLTITGTRAEDEADYYCESLVNDNTYVFGSGTQLTVL |
| 299 | scFv_29 VL | QSVLNQPPSVSVSLGQTATISCSGESLNKYYTQWFQQKAGQAPVLVIYKDTERP |
| | | SGIPDRFSGSSSGNTHTLTISGARAEDEADYYCESVDSTDTYVFGSGTQLTIL |
| 300 | scFv_30 VL | QSVLTQPASVSVSLGQTATISCSGESLSKYYTQWFQQKAGQAPVLVIYKDTERP |
| | | SGIPDRFSGSSSGNTHTLTISGAQAEDEADYYCQSYDDNVDGYVFGSGTQLTVL |
| 301 | scFv_31 VL | QSALTQPSSVSVSLGQTATISCSGESLTKYYVQWFQQKAGQAPVLVIYRDTERP |
| | | SGIPDRFSGSSSGNTHTLTISGARAEDEADYYCESMDGREAYVFGSGTQLTVL |
| 302 | scFv_32 VL | QSVLTQPASVSVSLGQTATISCSGESLSKYYAQWFQQKAGQAPVLVIYKDTERP |
| | | SGIPDRFSGSSSGNTHTLTITGIQAADEADYYCQSYDDNLDGYVFGSGTQLTVL |
| 303 | scFv_33 VL | DIVMTQTPLSLSVSPGEPASISCKASQSLLHSDGNTYLHWYLQKPGQSPQLLIYS |
| | | VSNRFTGVSDRFSGSGSGTDFTLRISRVEADDTGVYYCGQGTHLPFTFGQGTKL |
| | | EIK |
| 304 | scFv_34 VL | SYELTQPPSVSAALGQRVTISCTGTNTNIGSGYSVQWYQQLPGESPKPIIYGSSN |
| | | RPSGVPARFSGSKSGSTGTLTITGIQAEDEADYHCQSYDDSLDGHAVFGGGTQL |
| | | TIL |
| 305 | scFv_35 VL | SSVLTQPPSVSVSLGQTATISCSGESLSEYYAQWFQQKAGQAPVLVIYKDTERAS |
| | | GIPDRFSGSSSGNTHTLTISGARAEDEADYYCESAVNSDGIVFGGGTHLTVL |
| 306 | scFv_36 VL | QSVLTQPTSVSGSLGQRVTISCSGSTNNIGIVGASWYQQLPGKAPKLLVYSDGD |
| | | RPSGVPDRFSGSNSGNSDTLTITGLQAEDEADYYCQSFDTTLDAYVFGSGTQLT |
| | | VL |
| 307 | scFv_37 VL | SSVLTQPPSVSVSLGQTATISCSGESLNDYYAQWFQQKAGQVPVLVIYRDTERP |
| | | SGIPDRFSGSSSGNTHTLTISGARAEDEADYYCESEVSAGGAVFGEGTHLTVL |
| 308 | scFv_38 VL | SSVLTQPPSVSVSLGQTATISCSGESLSEYYAQWFQQKAGQAPVLVIYKDTERPS |
| | | GIPDRFSGSSSGNTHTLTISGARAEDEADYYCASADSSAIPVFGGGTHLTVL |
| 309 | scFv_39 VL | QSVLTQPPSVSGSPGQRVTISCSGTTKNIGIVGASWYQQLPGKAPKLLVHSDGF |
| | | RPSGVPDRFSASNSGDSDILTITGLQAEDEADYYCQSFDTTLDAHVFGSGTQLT |
| | | VL |
| 310 | scFv_40 VL | QSVLTQPPSVSGSLGQKVTISCTGSSSSVGTHYVGWYQIPPGIGPRTVIYNNDYR |
| | | PSGVPDRFSGSKSGNSATLTISGLQAEDEADYYCSSWDDSLGAYVLGSGTQLTI |
| | | L |
| 311 | scFv_41 VL | QPVLTQPPSVSVSLGQTATISCSGESLSKYYAQWFQQKAGQAPVLVIYKDTERP |
| | | SGIPDRFSGSSSGNTHTLTISGARAEDEADYYCESAVSTETVVFGGGTHLTVL |
| 312 | scFv_42 VL | QSVLTQPTSVSGSLGQRVTISCSGSTNDIGIIGAAWYQQLPGKAPKLLLYSNKNR |
| | | PSGVPDRFSGSNSGISATLTITGLQAEDEADYYCQSVDPALHTYVFGSGTQLTIL |
| 313 | scFv_43 VL | DIVMTQNPLSLAVTPGELATISCRASQSLLHSDGKSYLNWYLQKPGQTPRPLIY |
| | | EASRRFSGVSDRFNGSGSGTDFTLKISRVEAEDVGVYYCQQGLHFPPTFGAGT |
| | | KVELK |
| 314 | scFv_01 VH | EVQLVESGGDLVKPGGSLRLSCVASGFTFSNHEMYWVRQAPGKGLEWVARIY |
| | | DSGSRTRYADAVKGRFTISRDNAKNTLYLQMNSLTAEDTAVYFCAGGNSRGWT |
| | | EFGMDYWGPGTLVTVSS |
| 315 | scFv_02 VH | EEQLVEFGGDLVKPGGSLRLSCVASGFSFSSYDMSWVRQAPGKGLQWVASTNF |
| | | DGSRTYYTDAVEGRFTISRDNARNTVYLQMNSLRAEDTAVYYCARGMGSWG |
| | | ASTFGYWGQGTLVTVSS |
| 316 | scFv_03 VH | EVQLVQSGGDLVKPGGSLRLSCVASGFTFSNYTMSWVRQAPGQGLQWVAYIN |
| | | YDGSITYYAEAVKGRFTVSRDNAKNTLYLQMNSLRAEDTAVYFCVKETFMIPT |
| | | EWGQGTLITVSS |
| 317 | scFv_04 VH | EEQLVEFGGDLVKPGGSLRLSCVVSGFDFSRYGMSWVRQSPGKGLQWVADISY |
| | | RGTTFYADDVKGRFTVSRDSAKNTLYLEMTSLTVEDTAVYYCANSANWYVSP |
| | | FDHWGQGTLVTVSS |
| 318 | scFv_05 VH | EVQLVETGGDLVKPAGSLRLSCAASGFPFSGYSMTWVRQAPEKGLQLVAGINS |
| | | DGSHTYYTDDVKGRFTISRDNTKYILYLQMNSLRAEDTAMYYCGSHSWGNFH |
| | | YWGQGTLVTVSS |
| 319 | scFv_06 VH | EVQLVQSGGDLVKPGGSLRLSCVASGFTFSDNYMSWIRQVPGKGLQWVSRIST |
| | | DGRGTSYADAVKGRFTISRDNAKNTLYLQMNSLRDEDTAVYYCARRSGIWGFD |
| | | YWGQGALVTVSS |
| 320 | scFv_07 VH | EVQLVESGGDLVKPAGSLRLSCVASGFTFSNYGMNWVRQAPGKGLQWVAGIN |
| | | SGGSTTTYADVVKGRFTISRDNAKNTLYLQMNSLGAEDTAVYYCARVDYSSS |
| | | WLVDYWGQGTLVTVSS |
| 321 | scFv_08 VH | EVQLVESGGDLVKPGESLRLSCVASGFTFSSYGMSWVRQSPGKGLQWVADISY |
| | | GGGTYYADAVKGRFTISRDNARNTVYLQMNSLRAEDTAVYYCASQWGDWGH |
| | | FEYWGQGTLVTVSS |
| 322 | scFv_09 VH | EVQLVESGGDLVKPGGSLRLSCVASGFTFSSDSIHWVRQAPGKGLQWVAYINS |
| | | GGSTTYYADAVKGRFTISRDNARNTVYLKMNSLRAEDTAVYYCAKDSGFGSW |
| | | YLESWGQGTLVTVSP |
| 323 | scFv_10 VH | EVQLVQSGGDLVKPGGSLKISCVASGLTFNRNYMTWIRQAPGKGLQWVSEINP |
| | | DGSTTSYTDAVRGRFTISRDNAKNTLYLQMNSMRDEDTAVYYCARSSWGSFD |
| | | YWGQGTLVTVSS |
| 324 | scFv_11 VH | EVQLVESGGDLVKPGGSLRLSCVASGFTFSDYSMSWVRQSPGKGLQWVAAIMT |
| | | SGSTYYADAVKGRFTISRDNAKNTLYLQMDSLRAEDTAVYYCAHDYWFDFWG |
| | | QGTLVTVSS |
| 325 | scFv_12 VH | EEQLVEFGGDLVKPAGSLRLSCVASGFTFSSYDMSWVRQAPGKGLQWVATISY |
| | | DGSSTYYTDAVKGRFTVSRDNAKNTLYLQMNSLRAEDTALYYCAKDSGSWSH |
| | | FYGLDTWGHGTSVFVSS |
| 326 | scFv_13 VH | EVQLVESGGGLMKPGGSLKLSCLASGFTFSRYYMYWVRQAPGKGLQWVAQIS |
| | | SDGRRTYYADAVQGRFTISRDNVKNTVYLQMNSLSDEDTAVYYCVREDYFDF |
| | | DYWGQGTLVTVSS |
| 327 | scFv_14 VH | EVQLVQSGGDLVKPGGSLRLSCVASGFAFSGKTMQWVRQAPGKGLQWVAYIQ |
| | | HDGSRTSYADAVKGRFTISRDNAKNTLYLQMDSLRAEDTAVYYCADHPWYGP |
| | | EYWGQGTPVTVSS |
| 328 | scFv_15 VH | EVQLVQSGGDLVKPGGSLRLSCVASGFTFNKYHMSWVRQAPGKGLQWVGKIT |
| | | SDGSATGYADAVKGRFTISRDNAKNTFYLQMDSLRAEDTAMYYCARDQSSWG |
| | | TGNFDYWGQGTLVTVSS |
| 329 | scFv_16 VH | EVQLVESGGDLVKPGGSLRLSCVGSGFTLSSYWMSWVRQAPGKGLQWIAEISG |
| | | SGNTIYYGDAVKGRFTISRDNAKNTLYLQMNSLRDEDTAVYYCATEYYWNIDN |
| | | WGQGTLVTVSS |
| 330 | scFv_17 VH | EVPLVESGGDLVKPGGSLRLSCVASGFTFSSYGMSWVRQAPGKGLQWVAYING |
| | | GGSSTSYADAVRGRFTISRDNAENTLYLQMNSLRADDTAVYYCSNYYGIYWDS |
| | | DYWGQGTLVTVSS |
| 331 | scFv_18 VH | EVPLVESGGDLVKPGGSLRLSCVASGFTLSNYGMSWVRQSPGKGLQWVAAIRS |
| | | GGNTYYADAVKGRFTISRDNAKNTLYLQMNSLRAEDTAVYYCASQWGDWGH |
| | | FEYWGQGTLVTVSS |
| 332 | scFv_19 VH | EVQLVETGGDLVKPAGSLRLSCAASGFPFSGYSMTWVRQAPEKGLQLVAGINS |
| | | DGSHTYYTDDVKGRFTVSRDNTKYILYLQMNSLRVEDTAIYYCGSHSWGNFH |
| | | YWGQGTLVTVSS |
| 333 | scFv_20 VH | EVQLVESGGDLVKPGGSLRLSCVASGFTFSSYAMSWVRQAPGEGLQWVAYINS |
| | | AGSTTYYADAVKGRFTISRDNARNTVYLQMNSLRAEDTAVYYCGRYYYGLDY |
| | | WGPGTSVFVSS |
| 334 | scFv_21 VH | EVQLVESGGDMVKPGGSLRLSCVASGFTFSSFWMYWVRQAPGKGLQWVGGI |
| | | NSGGSITSYADAVKGRFTISRDNARNTLYLQMDSLRDEDTAVYYCAKPDIYGSF |
| | | DHWGQGTLVTVSS |
| 335 | scFv_22 VH | ELTLQESGPGLVKPAQTLSLTCTVSGDSVTSSHFWHWIRQRPGRGLEWMGYWS |
| | | DITIYSPTFQGRISITVDTARNQFSLHLNSVTTEDSAVYFCARYNGYVDYLDVW |
| | | GQGTLITVSS |
| 336 | scFv_23 VH | ELTLQESGPGLVKPSQTLSLTCVVSGGSVTTSDYWNWLRQRPGRGLEYMGYW |
| | | KGRTEYNPAFQGRISITADTAKNQFSLQLNSVTADDTAIYYCAHWGSGNLDYW |
| | | GPGTLVTVSS |
| 337 | scFv_24 VH | EVQLVQSGGXLVKPGGSLRLSCVASGFTLSSNYMDWVRQAPGKGPQWVAQIS |
| | | TDGSRTYYADAVKGRFTISRDTAKNTLYLQMNSLRAEDTAMYYCAKNEYLAP |
| | | TYWGQGTLVTVSS |
| 338 | scFv_25 VH | EEQLVEFGGDLVKPGGSLRLSCVASGFTFSSLSMNWIRQAPGKGLQWVSQISPD |
| | | GTITGYTDAVKGRFTISRDNAKNTLYLQMNSLRDEDTAIYYCAKSRYTDTYPFD |
| | | YWGPGTLVTVSS |
| 339 | scFv_26 VH | EVQLVETGGDLVKPGGSLRLSCVGSGFTFSSYWMSWVRQAPGKGLQWIADIS |
| | | DSGSSTDYADAVKGRFTISRDNAKNTLYLQMNSLRAEDTAMYYCARGLGMDY |
| | | WGHGTSLFVSS |
| 340 | scFv_27 VH | EVQLVESGGDLVKPGGSLRLSCVGSGFTFSSYWMSWVRQAPGKGLQWIADIS |
| | | DSGSSTDYADAVKGRFTISRDNAKNTLYLQMNSLRAEDTAMYYCARGLGMDY |
| | | WGHGTSLFVSS |
| 341 | scFv_28 VH | EVQLVETGGDLVKPGGSLRLSCVGSGFTFSSYWMSWVRQAPGKGLQWIADIS |
| | | DSGSSTDYADAVKGRFTISRDNAKNTLYLQMNSLRAEDTAMYYCARGLGMDY |
| | | WGHGTSLFVSS |
| 342 | scFv_29 VH | EVHLVESGGDLVKPGGSLRLSCVGSGFTFSSYWMSWVRQAPGKGLQWIADIS |
| | | DSGSSTDYADAVKGRFTISRDNAKNTLYLQMNSLRAEDTAMYYCARGLGMDY |
| | | WGHGTSLFVSS |
| 343 | scFv_30 VH | EVQLVESGGDLVKPGGSLRLSCVGSGFTFSSYWMSWVRQAPGKGLQWIADIS |
| | | DSGSSTDYADAVKGRFTISRDNAKNTLYLQMNSLRAEDTAMYYCARGLGMDY |
| | | WGHGTSLFVSS |
| 344 | scFv_31 VH | EVQLVESGGDLVKPGGSLRLSCVASGFTFSSYWMSWVRQAPGKGLQWIADISD |
| | | SGSSTDYADAVKGRFTISRDNAKNTLYLQMNSLRAEDTAMYYCARGLGMDY |
| | | WGHGTSLFVSS |
| 345 | scFv_32 VH | EVQLVESGGDLVKPGGSLRLSCVGSGFTFSSYWMSWVRQAPGKGLQWIADIS |
| | | DSGSSTDYADAVKGRFTISRDNAKNTLYLQMNSLRAEDTAMYYCARGLGMDY |
| | | WGHGTSLFVSS |
| 346 | scFv_33 VH | EVQLVESGGDLVKPGGSLRLSCAASGFTFSHYDMNWVRQAPGKGLQWVASIA |
| | | YDGSNTYYTDAVKGRFTISRDNAKNILFLLVNSLRAEDTAIYYCARGLVYWGQ |
| | | GTLVTVSS |
| 347 | scFv_34 VH | EVQLVESGGDLVKPGGSLRLSCVASGLTFSDNYMSWIRQVPGKGLQWVSRIST |
| | | DGRGTSYADAVKGRFTISRDNAKNTLYLQMNSLRDEDTAVYYCARRSGIWGFD |
| | | YWGQGTLVTVSS |
| 348 | scFv_35 VH | EVQLVESGGDLVKPAGSLRLSCVTSGFTFSSYAMSWVRQAPGKGLQWVGSINS |
| | | GGSTTSYADAVKGRFTISRDNAKNTVYLQMNSLRAEDTAVYYCARWWDFEY |
| | | WGQGTLVTVSS |
| 349 | scFv_36 VH | EEQLVEFGGGLVRPGGSLRLSCVASGFTFSSYGMTWVRQSPGKGLQWVATISD |
| | | TGSTYYADTVKGRFSISRDNAKNTLYLQMNSLRADDTALYYCTKDLGVWGMD |
| | | NWGPGTSLFVSS |
| 350 | scFv_37 VH | EVQLVETGGDLVKPGGSLRLSCVASGFTLRDYSMTWVRQTPEKGLQLVAGIST |
| | | GGSTTYYIDAVKGRFTISRDNAKNTVYLQMNSLRAEDTAMYHCTTTALGYWG |
| | | QGTLVTVSS |
| 351 | scFv_38 VH | EVQLVESGGDLVKPGGSLRLSCVASGFNFGNYAVSWVRQAPGKGLQWVAGISN |
| | | DGTITGYTDVVEGRFIISRDNAKNTLYLQMNSLRAEDMAIYYCVAGGFLDYWG |
| | | QGTLVTVSS |
| 352 | scFv_39 VH | EVPLVESGGDLVKPGGSLRLSCVASGFTFSSYGMSWVRQSPGKGLQWVAGITY |
| | | DGSNTYYTDTVRGRFTISRDNARNTVYLQMNSLRAEDTAVYYCVASPLYGTYG |
| | | FDYWGHGTSVFVSS |
| 353 | scFv_40 VH | ELTLQESGPGLVKPSQTLSLTCAVSGGSVIRGYYWTWIRQRPGRGLEWMGYWA |
| | | GSTDYNPAFQGRIFITAGTAKNQFSLEMTSMTTDDTAIYYCVGSLRGGWLFDN |
| | | WGQGTLVTVSS |
| 354 | scFv_41 VH | EVQLVETGGDLVMPAGSLRLSCVASGFTFSSSVMSWVRQAPGKGLEWVAGING |
| | | GGSFAAYGDAVKGRFTISRDNAKNTLYLQMNSLRAEDTAMYYCVGGGYMDF |
| | | WGPGTSLFVSS |
| 355 | scFv_42 VH | EVQLVEIGGDLVKPEGSLRLSCVVSGFTFSGYDMNWVRQAPGKGLQWVAYISS |
| | | DGRRTVYTDAVKGRFTISRDNAKNTLYLQMNSLKSEDTAVYYCVKGGWLDFW |
| | | GQGTLVTVSS |
| 356 | scFv_43 VH | EVQLVESGGDLVKPGGSLRLSCAASGFTFTDYDMSWVRQAPGKGLQWVAAIS |
| | | YDGSSTYYTD AVKGRFTISRDNARNTLYLQMSSLRAEDTAMYYCVVSNMNLW |
| | | GHGTSVFVSS |

### Anti-CD20 Binding Domains

The anti-CD20 binding domain can be any molecule that binds to CD20 with sufficient affinity and specificity, and is often an antibody or an antibody derivative, such as an scFv, single domain antibody (sdAb), Fab′ fragment, (Fab′)2 fragment, nanobody, diabody, or the like. As used herein, “sufficient affinity and specificity” can refer to the property that, under designated conditions, binding domain binds preferentially to its particular target protein (i.e., CD20) and does not bind in a significant amount to other proteins in a sample or subject. In certain embodiments, the anti-CD20 binding domain will have a sufficiently high binding affinity for CD20, for example, the antibody may bind CD20 with a Kd value of between 1E-07 to 1E-10 M. Antibody affinities may be determined, e.g., by a surface plasmon resonance based assay (such as the BIAcore® assay as described in PCT Application Publication No. WO2005/012359); enzyme-linked immunoabsorbent assay (ELISA); and competition assays (e.g. radioimmunoassays (RIAs)). Alternatively, the anti-CD20 binding domain can be a receptor or a receptor fragment that binds specifically to CD20. The anti-CD20 binding domain can be attached to the rest of the receptor directly (covalently) or indirectly (for example, through the noncovalent binding of two or more binding partners). Antibody derivatives are molecules that resemble antibodies in their mechanism of ligand binding, and include, for example, nanobodies, duobodies, diabodies, triabodies, minibodies, F(ab′)2 fragments, Fab fragments, single chain variable fragments (scFv), single domain antibodies (sdAb), synthetic binders, and functional fragments thereof. See for example, D. L. Porter et al., N Engl J Med (2011) 365(8):725-33 (scFv); E. L. Smith et al, Mol Ther (2018) 26(6): 1447-56 (scFv); S. R. Banihashemi et al., Iran J Basic Med Sci (2018) 21(5):455-64 (CD19 nanobody); F. Rahbarizadeh et al Adv Drug Deliv Rev (2019) 141:41-46 (sdAb); S. M. Kipriyanov et al., Int J Cancer (1998) 77(5):763-72 (diabody); F. Le Gall et al., FEBS Lett (1999) 453(1-2): 164-68 (triabody); M. A. Ghetie et al., Blood (1994) 83(5): 1329-36 (F(ab′)2); and M. A. Ghetie et al., Clin Cancer Res (1999) 5(12):3920-27 (F(ab′)2 and Fab′). Antibody derivatives can also be prepared from therapeutic antibodies, for example without limitation, by preparing a nanobody, duobody, diabody, triabody, minibody, F(ab′)2 fragment, Fab fragment, single chain variable fragment (scFv), or single domain antibody (sdAb) based on a therapeutic antibody. Antibody derivatives can also be designed using phage display techniques (see, e.g., E. Romao et al., Curr Pharm Des (2016) 22(43):6500-18).

In some embodiments, the anti-CD20 binding domain is an scFv.

Exemplary, non-limiting anti-CD20 sequences of some embodiments herein, including full anti-CD20 sequences, light chain sequences, heavy chain sequences, and CDR sequences can be found in U.S. Pat. No. 5,736,136, U.S. Pre-Grant Publication No. 2004/0167319, U.S. Pat. Nos. 9,539,251, 8,147,832, and WO 2006/130458, each of which is incorporated herein by reference in their entireties.

In some embodiments, the anti-CD20 amino acid sequence is encoded by a second nucleic acid sequence comprising a nucleic acid sequence comprising of one of SEQ ID NOS: 414-458 from Table 2A, or a functional variant of the nucleic acid sequence of one SEQ ID NOS: 414-458 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 414-458.

In some embodiments, the anti-CD20 amino acid sequence encoded by the second nucleic acid sequence comprises the amino acid sequence of one of SEQ ID NOS: 459-503 from Table 2A. In some embodiments the anti-CD20 amino acid sequence encoded by the second nucleic acid sequence comprises the amino acid sequence of a functional variant of one of SEQ ID NOS: 459-503 having at least about 70%, about 75%, about 80%, about 85%, about 90%, about 91%, about 92%, about 93%, about 94%, about 95%, about 96%, about 97%, about 98%, about 99% or more sequence identity to one SEQ ID NOS: 459-503 from Table 2A.

In some embodiments, the disclosure provides an optimized anti-CD20 binding domain comprising a codon optimized Leu16 sequence engineered to eliminate undesired recombination and cryptic splice products while preserving the native Leu16 amino acid sequence.

In some embodiments, the anti-CD20 amino acid sequence is encoded by a second nucleic acid sequence comprising a sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 799. In some embodiments, the anti-CD20 amino acid sequence is encoded by a second nucleic acid sequence comprising SEQ ID NO: 799.

TABLE 2A
Exemplary anti-CD20 scFv_nucleotide and amino acid sequences
(VL-linker-VH or VH-linker-VL)

| SEQ ID | | | |
|---|---|---|---|
| NO: | Name | Type | Sequence |
| 414 | scFv_44 | DNA | ATGGACTTCCAGGTGCAGATCATCAGCTTCCTGCTGATCAGCGCCAGC |
| | | | GTGATCATGAGCCGGGGCCAGATCGTGCTGAGCCAGAGCCCCGCCATC |
| | | | CTGAGCGCCAGCCCCGGCGAGAAGGTGACCATGACCTGCCGGGCCAG |
| | | | CAGCAGCGTGAGCTACATCCACTGGTTCCAGCAGAAGCCCGGCAGCA |
| | | | GCCCCAAGCCCTGGATCTACGCCACCAGCAACCTGGCCAGCGGCGTGC |
| | | | CCGTGCGGTTCAGCGGCAGCGGCAGCGGCACCAGCTACAGCCTGACC |
| | | | ATCAGCCGGGTGGAGGCCGAGGACGCCGCCACCTACTACTGCCAGCA |
| | | | GTGGACCAGCAACCCCCCCACCTTCGGCGGCGGCACCAAGCTGGAGA |
| | | | TCAAGGGCGGCGGCAGCAGCCGGAGCAGCAGCAGCGGCGGCGGCGG |
| | | | CAGCGGCGGCGGCGGCATGGGCTGGAGCCTGATCCTGCTGTTCCTGGT |
| | | | GGCCGTGGCCACCCGGGTGCTGAGCCAGGTGCAGCTGCAGCAGCCCG |
| | | | GCGCCGAGCTGGTGAAGCCCGGCGCCAGCGTGAAGATGAGCTGCAAG |
| | | | GCCAGCGGCTACACCTTCACCAGCTACAACATGCACTGGGTGAAGCAG |
| | | | ACCCCCGGCCGGGGCCTGGAGTGGATCGGCGCCATCTACCCCGGCAAC |
| | | | GGCGACACCAGCTACAACCAGAAGTTCAAGGGCAAGGCCACCCTGAC |
| | | | CGCCGACAAGAGCAGCAGCACCGCCTACATGCAGCTGAGCAGCCTGA |
| | | | CCAGCGAGGACAGCGCCGTGTACTACTGCGCCCGGAGCACCTACTACG |
| | | | GCGGCGACTGGTACTTCAACGTGTGGGGCGCCGGCACCACCGTGACC |
| | | | GTGAGCGCC |
| 459 | scFv_44 | AA | MDFQVQIISFLLISASVIMSRGQIVLSQSPAILSASPGEKVTMTCRASSSVSY |
| | | | IHWFQQKPGSSPKPWIYATSNLASGVPVRFSGSGSGTSYSLTISRVEAEDA |
| | | | ATYYCQQWTSNPPTFGGGTKLEIKGGGSSRSSSSGGGGSGGGGMGWSLI |
| | | | LLFLVAVATRVLSQVQLQQPGAELVKPGASVKMSCKASGYTFTSYNMHW |
| | | | VKQTPGRGLEWIGAIYPGNGDTSYNQKFKGKATLTADKSSSTAYMQLSSL |
| | | | TSEDSAVYYCARSTYYGGDWYFNVWGAGTTVTVSA |
| 415 | scFv_45 | DNA | GAGATCGTGCTGACCCAGAGCCCCGCCACCCTGAGCCTGAGCCCCGG |
| | | | CGAGCGGGCCACCCTGAGCTGCCGGGCCAGCCAGAGCGTGAGCAGCT |
| | | | ACCTGGCCTGGTACCAGCAGAAGCCCGGCCAGGCCCCCCGGCTGCTG |
| | | | ATCTACGACGCCAGCAACCGGGCCACCGGCATCCCCGCCCGGTTCAGC |
| | | | GGCAGCGGCAGCGGCACCGACTTCACCCTGACCATCAGCAGCCTGGA |
| | | | GCCCGAGGACTTCGCCGTGTACTACTGCCAGCAGCGGAGCAACTGGC |
| | | | CCATCACCTTCGGCCAGGGCACCCGGCTGGAGATCAAGGGCGGCGGC |
| | | | AGCAGCCGGAGCAGCAGCAGCGGCGGCGGCGGCAGCGGCGGCGGCG |
| | | | GCGAGGTGCAGCTGGTGGAGAGCGGCGGCGGCCTGGTGCAGCCCGGC |
| | | | CGGAGCCTGCGGCTGAGCTGCGCCGCCAGCGGCTTCACCTTCAACGA |
| | | | CTACGCCATGCACTGGGTGCGGCAGGCCCCCGGCAAGGGCCTGGAGT |
| | | | GGGTGAGCACCATCAGCTGGAACAGCGGCAGCATCGGCTACGCCGAC |
| | | | AGCGTGAAGGGCCGGTTCACCATCAGCCGGGACAACGCCAAGAAGAG |
| | | | CCTGTACCTGCAGATGAACAGCCTGCGGGCCGAGGACACCGCCCTGTA |
| | | | CTACTGCGCCAAGGACATCCAGTACG |
| 460 | scFv_45 | AA | EIVLTQSPATLSLSPGERATLSCRASQSVSSYLAWYQQKPGQAPRLLIYDA |
| | | | SNRATGIPARFSGSGSGTDFTLTISSLEPEDFAVYYCQQRSNWPITFGQGTR |
| | | | LEIKGGGSSRSSSSGGGGSGGGGEVQLVESGGGLVQPGRSLRLSCAASGF |
| | | | TFNDYAMHWVRQAPGKGLEWVSTISWNSGSIGYADSVKGRFTISRDNAK |
| | | | KSLYLQMNSLRAEDTALYYCAKDIQYGNYYYGMDVWGQGTTVTVSS |
| 416 | scFv_46 | DNA | GAGATCGTGCTGACCCAGAGCCCCGCCACCCTGAGCCTGAGCCCCGG |
| | | | CGAGCGGGCCACCCTGAGCTGCCGGGCCAGCCAGAGCGTGAGCAGCT |
| | | | ACCTGGCCTGGTACCAGCAGAAGCCCGGCCAGGCCCCCCGGCTGCTG |
| | | | ATCTACGACGCCAGCAACCGGGCCACCGGCATCCCCGCCCGGTTCAGC |
| | | | GGCAGCGGCAGCGGCACCGACTTCACCCTGACCATCAGCAGCCTGGA |
| | | | GCCCGAGGACTTCGCCGTGTACTACTGCCAGCAGCGGAGCAACTGGC |
| | | | CCATCACCTTCGGCCAGGGCACCCGGCTGGAGATCAAGGGCGGCGGC |
| | | | AGCAGCCGGAGCAGCAGCAGCGGCGGCGGCGGCAGCGGCGGCGGCG |
| | | | GCGAGGTGCAGCTGGTGGAGAGCGGCGGCGGCCTGGTGCAGCCCGAC |
| | | | CGGAGCCTGCGGCTGAGCTGCGCCGCCAGCGGCTTCACCTTCCACGA |
| | | | CTACGCCATGCACTGGGTGCGGCAGGCCCCCGGCAAGGGCCTGGAGT |
| | | | GGGTGAGCACCATCAGCTGGAACAGCGGCACCATCGGCTACGCCGAC |
| | | | AGCGTGAAGGGCCGGTTCACCATCAGCCGGGACAACGCCAAGAACAG |
| | | | CCTGTACCTGCAGATGAACAGCCTGCGGGCCGAGGACACCGCCCTGTA |
| | | | CTACTGCGCCAAGGACATCCAGTACGGCAACTACTACTACGGCATGGA |
| | | | CGTGTGGGGCCAGGGCACCACCGTGACCGTGAGCAGC |
| 461 | scFv_46 | AA | EIVLTQSPATLSLSPGERATLSCRASQSVSSYLAWYQQKPGQAPRLLIYDA |
| | | | SNRATGIPARFSGSGSGTDFTLTISSLEPEDFAVYYCQQRSNWPITFGQGTR |
| | | | LEIKGGGSSRSSSSGGGGSGGGGEVQLVESGGGLVQPDRSLRLSCAASGF |
| | | | TFHDYAMHWVRQAPGKGLEWVSTISWNSGTIGYADSVKGRFTISRDNAK |
| | | | NSLYLQMNSLRAEDTALYYCAKDIQYGNYYYGMDVWGQGTTVTVSS |
| 417 | scFv_47 | DNA | GAGATCGTGCTGACCCAGAGCCCCGCCACCCTGAGCCTGAGCCCCGG |
| | | | CGAGCGGGCCACCCTGAGCTGCCGGGCCAGCCAGAGCGTGAGCAGCT |
| | | | ACCTGGCCTGGTACCAGCAGAAGCCCGGCCAGGCCCCCCGGCTGCTG |
| | | | ATCTACGACGCCAGCAACCGGGCCACCGGCATCCCCGCCCGGTTCAGC |
| | | | GGCAGCGGCAGCGGCACCGACTTCACCCTGACCATCAGCAGCCTGGA |
| | | | GCCCGAGGACTTCGCCGTGTACTACTGCCAGCAGCGGAGCGACTGGC |
| | | | CCCTGACCTTCGGCGGCGGCACCAAGGTGGAGATCAAGGGCGGCGGC |
| | | | AGCAGCCGGAGCAGCAGCAGCGGCGGCGGCGGCAGCGGCGGCGGCG |
| | | | GCGAGGTGCAGCTGGTGCAGAGCGGCGGCGGCCTGGTGCACCCCGGC |
| | | | GGCAGCCTGCGGCTGAGCTGCACCGGCAGCGGCTTCACCTTCAGCTAC |
| | | | CACGCCATGCACTGGGTGCGGCAGGCCCCCGGCAAGGGCCTGGAGTG |
| | | | GGTGAGCATCATCGGCACCGGCGGCGTGACCTACTACGCCGACAGCGT |
| | | | GAAGGGCCGGTTCACCATCAGCCGGGACAACGTGAAGAACAGCCTGT |
| | | | ACCTGCAGATGAACAGCCTGCGGGCCGAGGACATGGCCGTGTACTACT |
| | | | GCGCCCGGGACTACTACGGCGCCGGCAGCTTCTACGACGGCCTGTACG |
| | | | GCATGGACGTGTGGGGCCAGGGCACCACCGTGACCGTGAGCAGC |
| 462 | scFv_47 | AA | EIVLTQSPATLSLSPGERATLSCRASQSVSSYLAWYQQKPGQAPRLLIYDA |
| | | | SNRATGIPARFSGSGSGTDFTLTISSLEPEDFAVYYCQQRSDWPLTFGGGTK |
| | | | VEIKGGGSSRSSSSGGGGSGGGGEVQLVQSGGGLVHPGGSLRLSCTGSGF |
| | | | TFSYHAMHWVRQAPGKGLEWVSIIGTGGVTYYADSVKGRFTISRDNVKN |
| | | | SLYLQMNSLRAEDMAVYYCARDYYGAGSFYDGLYGMDVWGQGTTVTV |
| | | | SS |
| 418 | scFv_48 | DNA | GACATCGTGATGACCCAGACCCCCCTGAGCCTGCCCGTGACCCCCGGC |
| | | | GAGCCCGCCAGCATCAGCTGCCGGAGCAGCAAGAGCCTGCTGCACAG |
| | | | CAACGGCATCACCTACCTGTACTGGTACCTGCAGAAGCCCGGCCAGAG |
| | | | CCCCCAGCTGCTGATCTACCAGATGAGCAACCTGGTGAGCGGCGTGCC |
| | | | CGACCGGTTCAGCGGCAGCGGCAGCGGCACCGACTTCACCCTGAAGA |
| | | | TCAGCCGGGTGGAGGCCGAGGACGTGGGCGTGTACTACTGCGCCCAG |
| | | | AACCTGGAGCTGCCCTACACCTTCGGCGGCGGCACCAAGGTGGAGAT |
| | | | CAAGCGGACCGTGGCCGCCCCCAGCGTGTTCATCTTCCCCCCCAGCGA |
| | | | CGAGCAGCTGAAGAGCGGCACCGCCAGCGTGGTGTGCCTGCTGAACA |
| | | | ACTTCTACCCCCGGGAGGCCAAGGTGCAGTGGAAGGTGGACAACGCC |
| | | | CTGCAGAGCGGCAACAGCCAGGAGAGCGTGACCGAGCAGGACAGCA |
| | | | AGGACAGCACCTACAGCCTGAGCAGCACCCTGACCCTGAGCAAGGCC |
| | | | GACTACGAGAAGCACAAGGTGTACGCCTGCGAGGTGACCCACCAGGG |
| | | | CCTGAGCAGCCCCGTGACCAAGAGCTTCAACCGGGGCGAGTGCGGCG |
| | | | GCGGCAGCAGCCGGAGCAGCAGCAGCGGCGGCGGCGGCAGCGGCGG |
| | | | CGGCGGCCAGGTGCAGCTGGTGCAGAGCGGCGCCGAGGTGAAGAAG |
| | | | CCCGGCAGCAGCGTGAAGGTGAGCTGCAAGGCCAGCGGCTACGCCTT |
| | | | CAGCTACAGCTGGATCAACTGGGTGCGGCAGGCCCCCGGCCAGGGCC |
| | | | TGGAGTGGATGGGCCGGATCTTCCCCGGCGACGGCGACACCGACTACA |
| | | | ACGGCAAGTTCAAGGGCCGGGTGACCATCACCGCCGACAAGAGCACC |
| | | | AGCACCGCCTACATGGAGCTGAGCAGCCTGCGGAGCGAGGACACCGC |
| | | | CGTGTACTACTGCGCCCGGAACGTGTTCGACGGCTACTGGCTGGTGTA |
| | | | CTGGGGCCAGGGCACCCTGGTGACCGTGAGCAGCGCCAGCACCAAGG |
| | | | GCCCCAGCGTGTTCCCCCTGGCCCCCAGCAGCAAGAGCACCAGCGGC |
| | | | GGCACCGCCGCCCTGGGCTGCCTGGTGAAGGACTACTTCCCCGAGCCC |
| | | | GTGACCGTGAGCTGGAACAGCGGCGCCCTGACCAGCGGCGTGCACAC |
| | | | CTTCCCCGCCGTGCTGCAGAGCAGCGGCCTGTACAGCCTGAGCAGCGT |
| | | | GGTGACCGTGCCCAGCAGCAGCCTGGGCACCCAGACCTACATCTGCA |
| | | | ACGTGAACCACAAGCCCAGCAACACCAAGGTGGACAAGAAGGTGGA |
| | | | GCCCAAGAGCTGCGACAAGACCCACACCTGCCCCCCCTGCCCCGCCC |
| | | | CCGAGCTGCTGGGCGGCCCCAGCGTGTTCCTGTTCCCCCCCAAGCCCA |
| | | | AGGACACCCTGATGATCAGCCGGACCCCCGAGGTGACCTGCGTGGTG |
| | | | GTGGACGTGAGCCACGAGGACCCCGAGGTGAAGTTCAACTGGTACGT |
| | | | GGACGGCGTGGAGGTGCACAACGCCAAGACCAAGCCCCGGGAGGAG |
| | | | CAGTACAACAGCACCTACCGGGTGGTGAGCGTGCTGACCGTGCTGCA |
| | | | CCAGGACTGGCTGAACGGCAAGGAGTACAAGTGCAAGGTGAGCAACA |
| | | | AGGCCCTGCCCGCCCCCATCGAGAAGACCATCAGCAAGGCCAAGGGC |
| | | | CAGCCCCGGGAGCCCCAGGTGTACACCCTGCCCCCCAGCCGGGACGA |
| | | | GCTGACCAAGAACCAGGTGAGCCTGACCTGCCTGGTGAAGGGCTTCT |
| | | | ACCCCAGCGACATCGCCGTGGAGTGGGAGAGCAACGGCCAGCCCGAG |
| | | | AACAACTACAAGACCACCCCCCCCGTGCTGGACAGCGACGGCAGCTT |
| | | | CTTCCTGTACAGCAAGCTGACCGTGGACAAGAGCCGGTGGCAGCAGG |
| | | | GCAACGTGTTCAGCTGCAGCGTGATGCACGAGGCCCTGCACAACCAC |
| | | | TACACCCAGAAGAGCCTGAGCCTGAGCCCCGGC |
| 463 | scFv_48 | AA | DIVMTQTPLSLPVTPGEPASISCRSSKSLLHSNGITYLYWYLQKPGQSPQLL |
| | | | IYQMSNLVSGVPDRFSGSGSGTDFTLKISRVEAEDVGVYYCAQNLELPYT |
| | | | FGGGTKVEIKRTVAAPSVFIFPPSDEQLKSGTASVVCLLNNFYPREAKVQ |
| | | | WKVDNALQSGNSQESVTEQDSKDSTYSLSSTLTLSKADYEKHKVYACEV |
| | | | THQGLSSPVTKSFNRGECGGGSSRSSSSGGGGSGGGGQVQLVQSGAEVK |
| | | | KPGSSVKVSCKASGYAFSYSWINWVRQAPGQGLEWMGRIFPGDGDTDY |
| | | | NGKFKGRVTITADKSTSTAYMELSSLRSEDTAVYYCARNVFDGYWLVYW |
| | | | GQGTLVTVSSASTKGPSVFPLAPSSKSTSGGTAALGCLVKDYFPEPVTVS |
| | | | WNSGALTSGVHTFPAVLQSSGLYSLSSVVTVPSSSLGTQTYICNVNHKPSN |
| | | | TKVDKKVEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVSVLT |
| | | | VLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPG |
| 419 | scFv_49 | DNA | CAGATCGTGCTGAGCCAGAGCCCCGCCATCCTGAGCGCCAGCCCCGGC |
| | | | GAGAAGGTGACCATGACCTGCCGGGCCAGCAGCAGCGTGAGCTACAT |
| | | | CCACTGGTTCCAGCAGAAGCCCGGCAGCAGCCCCAAGCCCTGGATCTA |
| | | | CGCCACCAGCAACCTGGCCAGCGGCGTGCCCGTGCGGTTCAGCGGCA |
| | | | GCGGCAGCGGCACCAGCTACAGCCTGACCATCAGCCGGGTGGAGGCC |
| | | | GAGGACGCCGCCACCTACTACTGCCAGCAGTGGACCAGCAACCCCCC |
| | | | CACCTTCGGCGGCGGCACCAAGCTGGAGATCAAGGGCGGCGGCAGCA |
| | | | GCCGGAGCAGCAGCAGCGGCGGCGGCGGCAGCGGCGGCGGCGGCCA |
| | | | GGTGCAGCTGCAGCAGCCCGGCGCCGAGCTGGTGAAGGCCGGCGCCA |
| | | | GCGTGAAGATGAGCTGCAAGGCCAGCGGCTACACCTTCACCAGCTAC |
| | | | AACATGCACTGGGTGAAGCAGACCCCCGGCCGGGGCCTGGAGTGGAT |
| | | | CGGCGCCATCTACCCCGGCAACGGCGACACCAGCTACAACCAGAAGT |
| | | | TCAAGGGCAAGGCCACCCTGACCGCCGACAAGAGCAGCAGCACCGCC |
| | | | TACATGCAGCTGAGCAGCCTGACCAGCGAGGACAGCGCCGTGTACTAC |
| | | | TGCGCCCGGAGCACCTACTACGGCGGCGACACCTACTTCAACGTGTGG |
| | | | GGCGCCGGCACCACCGTGACCGTGAGCGCC |
| 464 | scFv_49 | AA | QIVLSQSPAILSASPGEKVTMTCRASSSVSYIHWFQQKPGSSPKPWIYATSN |
| | | | LASGVPVRFSGSGSGTSYSLTISRVEAEDAATYYCQQWTSNPPTFGGGTK |
| | | | LEIKGGGSSRSSSSGGGGSGGGGQVQLQQPGAELVKAGASVKMSCKASG |
| | | | YTFTSYNMHWVKQTPGRGLEWIGAIYPGNGDTSYNQKFKGKATLTADKS |
| | | | SSTAYMQLSSLTSEDSAVYYCARSTYYGGDTYFNVWGAGTTVTVSA |
| 420 | scFv_50.1 | DNA | CAGATCGTGCTGAGCCAGAGCCCCGCCATCATCACCGCCAGCCCCGGC |
| | | | GAGAAGGTGACCATGACCTGCCGGGCCAGCACCAGCGCCAGCTACAT |
| | | | CCACTGGTTCCAGCAGAAGCCCACCAGCAGCCCCAAGCCCTGGATCTA |
| | | | CGCCACCAGCAACCTGGCCAGCGGCGTGCCCAGCCGGTTCAGCGGCA |
| | | | GCGGCAGCGGCACCACCTACAGCATGACCATCAGCAGCCTGGAGGCC |
| | | | GAGGACGCCGCCACCTACTACTGCCAGCAGTGGACCAGCAACCCCCC |
| | | | CACCTTCGGCGGCGGCACCAAGCTGGAGATCAAGGGCGGCGGCAGCA |
| | | | GCCGGAGCAGCAGCAGCGGCGGCGGCGGCAGCGGCGGCGGCGGCCA |
| | | | GGTGCAGCTGCAGCAGCCCGGCGCCGAGCTGAAGAAGCCCGGCGCCA |
| | | | GCGTGAAGGTGAGCTGCAAGGCCAGCGGCTACACCTTCACCAGCTAC |
| | | | AACATGCACTGGGTGAAGCAGACCCCCGGCCGGGGCCTGGAGTGGAC |
| | | | CGGCGCCATCTACCCCGGCAACGGCGACACCAGCTACAACCAGAAGT |
| | | | TCAAGGGCAAGACCACCCTGACCGCCGACAAGAGCAGCAGCACCGCC |
| | | | TACATGGAGCTGAGCAGCCTGCGGAGCGAGGACACCGCCGTGTACTA |
| | | | CTGCGCCCGGAGCACCTACTACGGCGGCGACTGGTACTTCAACGTGTG |
| | | | GGGCGCCGGCACCACCGTGACCGTGAGCGCC |
| 465 | scFv_50.1 | AA | QIVLSQSPAIITASPGEKVTMTCRASTSASYIHWFQQKPTSSPKPWIYATSN |
| | | | LASGVPSRFSGSGSGTTYSMTISSLEAEDAATYYCQQWTSNPPTFGGGTK |
| | | | LEIKGGGSSRSSSSGGGGSGGGGQVQLQQPGAELKKPGASVKVSCKASG |
| | | | YTFTSYNMHWVKQTPGRGLEWTGAIYPGNGDTSYNQKFKGKTTLTADK |
| | | | SSSTAYMELSSLRSEDTAVYYCARSTYYGGDWYFNVWGAGTTVTVSA |
| 421 | scFv_50.2 | DNA | CAGATCGTGCTGAGCCAGAGCCCCGCCATCATCACCGCCAGCCCCGGC |
| | | | GAGAAGGTGACCATGACCTGCCGGGCCAGCACCAGCGTGAGCTACAT |
| | | | CCACTGGTTCCAGCAGAAGCCCACCAGCAGCCCCAAGCCCTGGATCTA |
| | | | CGCCACCAGCAACCTGGCCAGCGGCGTGCCCAGCCGGTTCAGCGGCA |
| | | | GCGGCAGCGGCACCACCTACAGCATGACCATCAGCAGCCTGGAGGCC |
| | | | GAGGACGCCGCCACCTACTACTGCCAGCAGTGGACCAGCAACCCCCC |
| | | | CACCTTCGGCGGCGGCACCAAGCTGGAGATCAAGGGCGGCGGCAGCA |
| | | | GCCGGAGCAGCAGCAGCGGCGGCGGCGGCAGCGGCGGCGGCGGCCA |
| | | | GGTGCAGCTGCAGCAGCCCGGCGCCGAGCTGAAGAAGCCCGGCGCCA |
| | | | GCGTGAAGGTGAGCTGCAAGGCCAGCGGCTACACCTTCACCAGCTAC |
| | | | AACATGCACTGGGTGAAGCAGACCCCCGGCCGGGGCCTGGAGTGGAT |
| | | | CGGCGCCATCTACCCCGGCAACGGCGACACCAGCTACAACCAGAAGT |
| | | | TCAAGGGCAAGACCACCCTGACCGCCGACAAGAGCAGCAGCACCGCC |
| | | | TACATGGAGCTGAGCAGCCTGCGGAGCGAGGACACCGCCGTGTACTA |
| | | | CTGCGCCCGGAGCACCTACTACGGCGGCGACTGGTACTTCAACGTGTG |
| | | | GGGCGCCGGCACCACCGTGACCGTGAGCGCC |
| 466 | scFv_50.2 | AA | QIVLSQSPAIITASPGEKVTMTCRASTSVSYIHWFQQKPTSSPKPWIYATSN |
| | | | LASGVPSRFSGSGSGTTYSMTISSLEAEDAATYYCQQWTSNPPTFGGGTK |
| | | | LEIKGGGSSRSSSSGGGGSGGGGQVQLQQPGAELKKPGASVKVSCKASG |
| | | | YTFTSYNMHWVKQTPGRGLEWIGAIYPGNGDTSYNQKFKGKTTLTADKS |
| | | | SSTAYMELSSLRSEDTAVYYCARSTYYGGDWYFNVWGAGTTVTVSA |
| 422 | scFv_51 | DNA | GACATCGTGCTGACCCAGAGCCCCGCCATCCTGAGCGCCAGCCCCGGC |
| | | | GAGAAGGTGACCATGACCTGCCGGGCCAGCAGCAGCGTGAACTACAT |
| | | | GGACTGGTACCAGAAGAAGCCCGGCAGCAGCCCCAAGCCCTGGATCT |
| | | | ACGCCACCAGCAACCTGGCCAGCGGCGTGCCCGCCCGGTTCAGCGGC |
| | | | AGCGGCAGCGGCACCAGCTACAGCCTGACCATCAGCCGGGTGGAGGC |
| | | | CGAGGACGCCGCCACCTACTACTGCCAGCAGTGGAGCTTCAACCCCCC |
| | | | CACCTTCGGCGGCGGCACCAAGCTGGAGATCAAGGGCGGCGGCAGCA |
| | | | GCCGGAGCAGCAGCAGCGGCGGCGGCGGCAGCGGCGGCGGCGGCGA |
| | | | GGTGCAGCTGCAGCAGAGCGGCGCCGAGCTGGTGAAGCCCGGCGCCA |
| | | | GCGTGAAGATGAGCTGCAAGGCCAGCGGCTACACCTTCACCAGCTAC |
| | | | AACATGCACTGGGTGAAGCAGACCCCCGGCCAGGGCCTGGAGTGGAT |
| | | | CGGCGCCATCTACCCCGGCAACGGCGACACCAGCTACAACCAGAAGT |
| | | | TCAAGGGCAAGGCCACCCTGACCGCCGACAAGAGCAGCAGCACCGCC |
| | | | TACATGCAGCTGAGCAGCCTGACCAGCGAGGACAGCGCCGACTACTA |
| | | | CTGCGCCCGGAGCAACTACTACGGCAGCAGCTACTGGTTCTTCGACGT |
| | | | GTGGGGCGCCGGCACCACCGTGACCGTGAGCAGC |
| 467 | scFv_51 | AA | DIVLTQSPAILSASPGEKVTMTCRASSSVNYMDWYQKKPGSSPKPWIYAT |
| | | | SNLASGVPARFSGSGSGTSYSLTISRVEAEDAATYYCQQWSFNPPTFGGGT |
| | | | KLEIKGGGSSRSSSSGGGGSGGGGEVQLQQSGAELVKPGASVKMSCKAS |
| | | | GYTFTSYNMHWVKQTPGQGLEWIGAIYPGNGDTSYNQKFKGKATLTAD |
| | | | KSSSTAYMQLSSLTSEDSADYYCARSNYYGSSYWFFDVWGAGTTVTVSS |
| 819 | Leu16 | AA | EVQLQQSGAELVKPGASVKMSCKASGYTFTSYNMHWVKQTPGQGLEWI |
| | VH- | | GAIYPGNGDTSYNQKFKGKATLTADKSSSTAYMQLSSLTSEDSADYYCAR |
| | VL | | SNYYGSSYWFFDVWGAGTTVTVSSGSTSGGGSGGGSGGGSSDIVLTQSP |
| | | | AILSASPGEKVTMTCRASSSVNYMDWYQKKPGSSPKPWIYATSNLASGV |
| | | | PARFSGSGSGTSYSLTISRVEAEDAATYYCQQWSFNPPTFGGGTKLEIK |
| 820 | Leu-16 | DNA | GAGGTGCAGCTGCAGCAGTCTGGGGCTGAGCTGGTGAAGCCTGGGGC |
| | VH- | | CTCAGTGAAGATGTCCTGCAAGGCTTCTGGCTACACATTTACCAGTTAC |
| | VL | | AATATGCACTGGGTAAAGCAGACACCTGGACAGGGCCTGGAATGGATT |
| | | | GGAGCTATTTATCCAGGAAATGGTGATACTTCCTACAATCAGAAGTTCA |
| | | | AAGGCAAGGCCACATTGACTGCAGACAAATCCTCCAGCACAGCCTAC |
| | | | ATGCAGCTCAGCAGCCTGACATCTGAGGACTCTGCGGACTATTACTGT |
| | | | GCAAGATCTAATTATTACGGTAGTAGCTACTGGTTCTTCGATGTCTGGG |
| | | | GCGCAGGGACCACGGTCACCGTCTCCTCAGGCAGTACTAGCGGTGGT |
| | | | GGCTCCGGGGGCGGTTCCGGTGGGGGCGGCAGCAGCGACATTGTGCT |
| | | | GACCCAATCTCCAGCTATCCTGTCTGCATCTCCAGGGGAGAAGGTCAC |
| | | | AATGACTTGCAGGGCCAGCTCAAGTGTAAATTACATGGACTGGTACCA |
| | | | GAAGAAGCCAGGTTCCTCCCCCAAACCCTGGATTTATGCCACATCCAA |
| | | | CCTGGCTTCTGGAGTCCCTGCTCGCTTCAGTGGCAGTGGGTCTGGGAC |
| | | | CTCTTACTCTCTCACAATCAGCAGAGTGGAGGCTGAAGATGCTGCCAC |
| | | | TTATTACTGCCAGCAGTGGAGTTTTAATCCACCCACGTTCGGAGGGGG |
| | | | GACCAAGCTGGAAATAAAA |
| 799 | CODON | DNA | GAGGTGCAGCTGCAGCAGTCTGGGGCTGAGCTGGTGAAGCCTGGGGC |
| | OPTIMIZED | | CTCAGTGAAGATGTCCTGCAAGGCTTCTGGCTACACATTTACCAGTTAC |
| | VH- | | AATATGCACTGGGTAAAGCAGACACCTGGACAGGGCCTGGAATGGATT |
| | -VL | | GGAGCTATTTATCCAGGAAATGGTGATACTTCCTACAATCAGAAGTTCA |
| | | | AAGGCAAGGCCACATTGACTGCAGACAAATCCTCCAGCACAGCCTAC |
| | | | ATGCAGCTCAGCAGCCTGACATCTGAGGACAGCGCGGATTACTACTGC |
| | | | GCTAGGTCCAATTATTATGGCTCCTCATACTGGTTCTTCGATGTCTGGGG |
| | | | CGCAGGGACCACGGTCACCGTCTCCTCAGGCAGTACTAGCGGTGGTG |
| | | | GCTCCGGGGGCGGTTCCGGTGGGGGGGGCAGCAGCGACATTGTGCTG |
| | | | ACCCAATCTCCAGCTATCCTGTCTGCATCTCCAGGGGAGAAGGTCACA |
| | | | ATGACTTGCAGGGCCAGCTCAAGTGTAAATTACATGGACTGGTACCAG |
| | | | AAGAAGCCAGGATCCTCCCCCAAACCCTGGATTTATGCCACATCCAAC |
| | | | CTGGCATCTGGAGTCCCTGCTCGCTTCAGTGGCAGTGGGTCTGGGACC |
| | | | TCTTACTCTCTCACAATCAGCAGAGTGGAGGCTGAAGATGCTGCCACT |
| | | | TATTACTGCCAGCAGTGGAGTTTTAATCCACCCACGTTCGGAGGGGGG |
| | | | ACCAAGCTGGAAATAAAA |
| 423 | scFv_51.1 | DNA | GACATCGTGCTGACCCAGAGCCCCGCCATCATCACCGCCAGCCCCGGC |
| | | | GAGAAGGTGACCATGACCTGCCGGGCCAGCAGCAGCGTGAACTACAT |
| | | | GGACTGGTACCAGAAGAAGCCCGGCAGCAGCCCCAAGCCCTGGATCT |
| | | | ACGCCACCAGCAACCTGGCCAGCGGCGTGCCCAGCCGGTTCAGCGGC |
| | | | AGCGGCAGCGGCACCACCTACAGCATGACCATCAGCAGCCTGGAGGC |
| | | | CGAGGACGCCGCCACCTACTACTGCCAGCAGTGGAGCTTCAACCCCCC |
| | | | CACCTTCGGCGGCGGCACCAAGCTGGAGATCAAGGGCGGCGGCAGCA |
| | | | GCCGGAGCAGCAGCAGCGGCGGCGGCGGCAGCGGCGGCGGCGGCGA |
| | | | GGTGCAGCTGCAGCAGAGCGGCGCCGAGCTGAAGAAGCCCGGCGCC |
| | | | AGCGTGAAGGTGAGCTGCAAGGCCAGCGGCTACACCTTCACCAGCTA |
| | | | CAACATGCACTGGGTGAAGCAGACCCCCGGCCAGGGCCTGGAGTGGA |
| | | | TCGGCGCCATCTACCCCGGCAACGGCGACACCAGCTACAACCAGAAG |
| | | | TTCAAGGGCAAGACCACCCTGACCGCCGACAAGAGCAGCAGCACCGC |
| | | | CTACATGGAGCTGAGCAGCCTGCGGAGCGAGGACACCGCCGTGTACT |
| | | | ACTGCGCCCGGAGCAACTACTACGGCAGCAGCTACTGGTTCTTCGACG |
| | | | TGTGGGGCACCGGCACCACCGTGACCGTGAGCAGC |
| 468 | scFv_51.1 | AA | DIVLTQSPAIITASPGEKVTMTCRASSSVNYMDWYQKKPGSSPKPWIYATS |
| | | | NLASGVPSRFSGSGSGTTYSMTISSLEAEDAATYYCQQWSFNPPTFGGGT |
| | | | KLEIKGGGSSRSSSSGGGGSGGGGEVQLQQSGAELKKPGASVKVSCKAS |
| | | | GYTFTSYNMHWVKQTPGQGLEWIGAIYPGNGDTSYNQKFKGKTTLTAD |
| | | | KSSSTAYMELSSLRSEDTAVYYCARSNYYGSSYWFFDVWGTGTTVTVSS |
| 424 | scFv_52 | DNA | CAGATCGTGCTGAGCCAGAGCCCCACCATCCTGAGCGCCAGCCCCGGC |
| | | | GAGAAGGTGACCATGACCTGCCGGGCCAGCAGCAGCGTGAGCTACAT |
| | | | GGACTGGTACCAGCAGAAGCCCGGCAGCAGCCCCAAGCCCTGGATCT |
| | | | ACGCCACCAGCAACCTGGCCAGCGGCGTGCCCGCCCGGTTCAGCGGC |
| | | | AGCGGCAGCGGCACCAGCTACAGCCTGACCATCAGCCGGGTGGAGGC |
| | | | CGAGGACGCCGCCACCTACTACTGCCAGCAGTGGATCAGCAACCCCCC |
| | | | CACCTTCGGCGCCGGCACCAAGCTGGAGCTGGGCGGCGGCAGCAGCC |
| | | | GGAGCAGCAGCAGCGGCGGCGGCGGCAGCGGCGGCGGCGGCCAGGT |
| | | | GCAGCTGCAGCAGCCCGGCGCCGAGCTGGTGAAGCCCGGCGCCAGCG |
| | | | TGAAGATGAGCTGCAAGACCAGCGGCTACACCTTCACCAGCTACAAC |
| | | | GTGCACTGGGTGAAGCAGACCCCCGGCCAGGGCCTGGAGTGGATCGG |
| | | | CGCCATCTACCCCGGCAACGGCGACACCAGCTTCAACCAGAAGTTCAA |
| | | | GGGCAAGGCCACCCTGACCGCCGACAAGAGCAGCAGCACCGTGTACA |
| | | | TGCAGCTGAGCAGCCTGACCAGCGAGGACAGCGCCGTGTACTACTGC |
| | | | GCCCGGAGCAACTACTACGGCAGCAGCTACGTGTGGTTCTTCGACGTG |
| | | | TGGGGCGCCGGCACCACCGGCACCGGCAGCAGC |
| 469 | scFv_52 | AA | QIVLSQSPTILSASPGEKVTMTCRASSSVSYMDWYQQKPGSSPKPWIYATS |
| | | | NLASGVPARFSGSGSGTSYSLTISRVEAEDAATYYCQQWISNPPTFGAGTK |
| | | | LELGGGSSRSSSSGGGGSGGGGQVQLQQPGAELVKPGASVKMSCKTSGY |
| | | | TFTSYNVHWVKQTPGQGLEWIGAIYPGNGDTSFNQKFKGKATLTADKSS |
| | | | STVYMQLSSLTSEDSAVYYCARSNYYGSSYVWFFDVWGAGTTGTGSS |
| 425 | scFv_53 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGTACAG |
| | | | CGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCACCCGGCAGGTGTTCGAGCCCGCCCTGGAGACCGACTTCGTG |
| | | | GGCGGCGGCGTGGGCTACACCTACAAGTGCGTGGTGGAGCAGATCGC |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | CGGTACCGGCCCAGCTTCCAGGGCCAGGTGACCATCAGCGCCGACAA |
| | | | GAGCATCACCACCGCCTACCTGCAGTGGAGCAGCCTGAAGGCCAGCG |
| | | | ACACCGCCATGTACTACTGCGCCCGGCACCCCAGCTACGGCAGCGGCA |
| | | | GCCCCAACTTCGACTACTGGGGCCAGGGCACCCTGGTGACCGTGAGC |
| | | | AGC |
| 470 | scFv_53 | AA | DIVMTQTPLSSPVTLGQPASISCRSSQSLVYSDGNTYLSWLQQRPGQPPRL |
| | | | LIYKISNRFSGVPDRFSGSGAGTDFTLKISTRQVFEPALETDFVGGGVGYT |
| | | | YKCVVEQIAKGGGSSRSSSSGGGGSGGGGEVQLVQSGAEVKKPGESLKIS |
| | | | CKGSGYSFTSYWIGWVRQMPGKGLEWMGIIYPGDSDTRYRPSFQGQVTI |
| | | | SADKSITTAYLQWSSLKASDTAMYYCARHPSYGSGSPNFDYWGQGTLVT |
| | | | VSS |
| 426 | scFv_54 | DNA | GAGATCGTGATGACCCAGAGCCCCGCCACCCTGAGCGTGAGCCCCGG |
| | | | CGAGCGGGCCACCCTGAGCTGCCGGGCCAGCCAGAGCGTGAGCAGCA |
| | | | ACCTGGCCTGGTACCAGCAGAAGCCCGGCCAGGCCCCCCGGCTGCTG |
| | | | ATCAGCGGCGCCAGCACCCGGGCCACCGGCATCCCCGCCCGGTTCAGC |
| | | | GGCAGCGGCAGCGGCACCGAGTTCACCCTGACCATCAGCAGCCTGCA |
| | | | GAGCGAGGACTTCGCCGTGTACTACTGCCACCAGTACAACGACTGGA |
| | | | GCCTGACCTTCGGCGGCGGCACCAAGGTGGAGATCAAGGGCGGCGGC |
| | | | AGCAGCCGGAGCAGCAGCAGCGGCGGCGGCGGCAGCGGCGGCGGCG |
| | | | GCAGCTGCAAGGGCAGCGGCTACAGCTTCACCAGCTACTGGATCGGCT |
| | | | GGGTGCGGCAGATGCCCGGCAAGGGCCTGGAGTGGATGGGCATCATCT |
| | | | ACCCCGGCGACAGCGACACCCGGTACAGCCCCAGCTTCCAGGGCCAG |
| | | | GTGACCATCAGCGCCGACAAGAGCATCAGCACCGCCTACCTGCAGTGG |
| | | | AGCAGCCTGAAGGCCAGCGACACCGCCATGTACTACTGCGCCCGGATG |
| | | | GGCGACTTCTGGAGCGGCTACTACACCCGGGGCATGGACGTGTGGGG |
| | | | CCAGGGCACCACCGTGACCGTGAGCAGC |
| 471 | scFv_54 | AA | EIVMTQSPATLSVSPGERATLSCRASQSVSSNLAWYQQKPGQAPRLLISGA |
| | | | STRATGIPARFSGSGSGTEFTLTISSLQSEDFAVYYCHQYNDWSLTFGGGTK |
| | | | VEIKGGGSSRSSSSGGGGSGGGGSCKGSGYSFTSYWIGWVRQMPGKGLE |
| | | | WMGIIYPGDSDTRYSPSFQGQVTISADKSISTAYLQWSSLKASDTAMYYC |
| | | | ARMGDFWSGYYTRGMDVWGQGTTVTVSS |
| 427 | scFv_55 | DNA | GACATCGTGATGACCCAGACCCCCCTGAGCAGCCCCGTGACCCTGGGC |
| | | | CAGCCCGCCAGCATCAGCTGCCGGAGCAGCCAGCGGCTGGTGCACAG |
| | | | CGACGGCCACACCTACCTGAGCTGGCTGCAGCAGCGGCCCGGCCAGC |
| | | | CCCCCCGGCTGCTGATCAGCAAGGTGAGCAACCGGTTCAGCGGCGTG |
| | | | CCCGACCGGTTCAGCGGCAGCGGCGCCGGCACCGACTTCACCCTGAA |
| | | | GATCAGCCGGGTGGAGGCCGAGGACGTGGGCATCTACTACTGCATGCA |
| | | | GGCCACCCAGTTCCCCCTGACCTTCGGCGGCGGCACCAAGGTGGAGA |
| | | | TCAAGGGCGGCGGCAGCAGCCGGAGCAGCAGCAGCGGCGGCGGCGG |
| | | | CAGCGGCGGCGGCGGCGAGGTGCAGCTGGTGCAGAGCGGCGCCGAG |
| | | | GTGAAGAAGCCCGGCGAGAGCCTGAAGATCAGCTGCAAGGGCAGCG |
| | | | GCTACAGCTTCACCAACTACTGGATCGTGTGGGTGCGGCAGATGCCCG |
| | | | GCAAGGGCCTGGAGTGGATGGGCATCATCTACCCCGGCGACAGCGAC |
| | | | ACCCGGTACAGCCCCAGCTTCCAGGGCCAGGTGACCATCAGCGCCGA |
| | | | CAAGAGCATCAGCACCGCCTACCTGCAGTGGAGCAGCCTGAAGGCCA |
| | | | GCGACACCGCCATGTACTACTGCGCCCGGCACGGCGACTACTACTACT |
| | | | ACTACGGCATGGACGTGTGGGGCCAGGGCACCACCGTGACCGTGAGC |
| | | | AGC |
| 472 | scFv_55 | AA | DIVMTQTPLSSPVTLGQPASISCRSSQRLVHSDGHTYLSWLQQRPGQPPRL |
| | | | LISKVSNRFSGVPDRFSGSGAGTDFTLKISRVEAEDVGIYYCMQATQFPLT |
| | | | FGGGTKVEIKGGGSSRSSSSGGGGSGGGGEVQLVQSGAEVKKPGESLKIS |
| | | | CKGSGYSFTNYWIVWVRQMPGKGLEWMGIIYPGDSDTRYSPSFQGQVTI |
| | | | SADKSISTAYLQWSSLKASDTAMYYCARHGDYYYYYGMDVWGQGTTV |
| | | | TVSS |
| 428 | scFv_56 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGTACAG |
| | | | CGACGGCAACACCTACCTGAGCTGGCTGCAGCAGCGGCCCGGCCAGC |
| | | | CCCCCCGGCTGCTGTTCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCGTGCA |
| | | | GGCCACCCAGTTCCCCCTGACCTTCGGCGGCGGCACCAAGGTGGAGA |
| | | | TCAAGGGCGGCGGCAGCAGCCGGAGCAGCAGCAGCGGCGGCGGCGG |
| | | | CAGCGGCGGCGGCGGCGAGGTGCAGCTGGTGCAGAGCGGCGCCGAG |
| | | | GTGAAGAAGCCCGGCGAGAGCCTGAAGATCAGCTGCAAGGGCAGCG |
| | | | GCTACAGCTTCACCAGCTACTGGATCGGCTGGGTGCGGCAGATGAGCG |
| | | | GCAAGGGCCTGGAGTGGATGGGCATCATCTACCCCGGCGACAGCGAC |
| | | | ACCCGGTACAGCCCCAGCTTCCAGGGCCAGGTGACCATCAGCGCCGA |
| | | | CAAGAGCATCAGCACCGCCTACCTGCAGTGGAGCAGCCTGAAGGCCA |
| | | | GCGACACCGCCATCTACTACTGCGCCCGGCACCCCAGCTACGGCAGCG |
| | | | GCAGCCCCAACTTCGACTACTGGGGCCAGGGCACCCTGGTGACCGTG |
| | | | AGCAGC |
| 473 | scFv_56 | AA | DIVMTQTPLSSPVTLGQPASISCRSSQSLVYSDGNTYLSWLQQRPGQPPRL |
| | | | LFYKISNRFSGVPDRFSGSGAGTDFTLKISRVEAEDVGVYYCVQATQFPLT |
| | | | FGGGTKVEIKGGGSSRSSSSGGGGSGGGGEVQLVQSGAEVKKPGESLKIS |
| | | | CKGSGYSFTSYWIGWVRQMSGKGLEWMGIIYPGDSDTRYSPSFQGQVTI |
| | | | SADKSISTAYLQWSSLKASDTAIYYCARHPSYGSGSPNFDYWGQGTLVTV |
| | | | SS |
| 429 | scFv_57 | DNA | GACGTGGTGATGACCCAGAGCCCCCTGAGCCTGAGCGTGACCCTGGG |
| | | | CCAGCCCGCCAGCATCAGCTGCCGGAGCAGCCAGAGCCTGGTGTACA |
| | | | ACGACGGCGACACCTACCTGAACTGGTTCCAGCAGCGGCCCGGCCAG |
| | | | AGCCCCCGGCTGCTGATCTACAAGGTGAGCAACTGGGACAGCGGCGT |
| | | | GCCCGACCGGTTCAGCGGCAGCGGCAGCGGCACCGACTTCACCCTGA |
| | | | AGGTGAGCCGGGTGGAGGCCGAGGACGTGGGCGTGTACTACTGCATG |
| | | | CAGGGCACCCACTGGCCCCTGACCTTCGGCGGCGGCACCAAGGTGGA |
| | | | GATCAAGGGCGGCGGCAGCAGCCGGAGCAGCAGCAGCGGCGGCGGC |
| | | | GGCAGCGGCGGCGGCGGCGAGGTGCAGCTGGTGCAGAGCGGCGCCG |
| | | | AGGTGAAGAAGCCCGGCGAGAGCCTGAAGATCAGCTGCAAGGGCAG |
| | | | CGGCTACAGCTTCACCAACTACTGGATCGGCTGGGTGCGGCAGATGCC |
| | | | CGGCAAGGGCCTGGAGTGGATGGGCATCATCTACCCCGGCGACAGCG |
| | | | ACACCCGGTACAGCCCCAGCTTCCAGGGCCAGGTGACCATCAGCGCC |
| | | | GACAAGAGCATCAGCACCGCCTACCTGCAGTGGAGCAGCCTGAAGGC |
| | | | CAGCGACACCGCCATGTACTACTGCGCCCGGCAGGGCGACTACTACCA |
| | | | CTACAGCGGCATGGACGTGTGGGGCCAGGGCACCACCGTGACCGTGA |
| | | | GCAGC |
| 474 | scFv_57 | AA | DVVMTQSPLSLSVTLGQPASISCRSSQSLVYNDGDTYLNWFQQRPGQSPR |
| | | | LLIYKVSNWDSGVPDRFSGSGSGTDFTLKVSRVEAEDVGVYYCMQGTH |
| | | | WPLTFGGGTKVEIKGGGSSRSSSSGGGGSGGGGEVQLVQSGAEVKKPGE |
| | | | SLKISCKGSGYSFTNYWIGWVRQMPGKGLEWMGIIYPGDSDTRYSPSFQ |
| | | | GQVTISADKSISTAYLQWSSLKASDTAMYYCARQGDYYHYSGMDVWGQ |
| | | | GTTVTVSS |
| 430 | scFv_58 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAAGACCTACCTGAGCTGGCTGCAGCAGCGGCCCGGCCAGC |
| | | | CCCCCCGGCTGCTGATCTACAAGAGCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCGTGCA |
| | | | GGCCACCCAGTTCCCCATCACCTTCGGCCAGGGCACCCGGCTGGAGAT |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCAGCACCGCCTACCTGCAGTGGAGCAGCCTGAAGGCCAGCG |
| | | | ACACCGCCATGTACTACTGCGCCCGGCTGGGCGACTACTACAACTACT |
| | | | ACGGCATGGACGTGTGGGGCCAGGGCACCACCGTGACCGTGAGCAGC |
| 475 | scFv_58 | AA | DIVMTQTPLSSPVTLGQPASISCRSSQSLVHSDGKTYLSWLQQRPGQPPRL |
| | | | LIYKSSNRFSGVPDRFSGSGAGTDFTLKISRVEAEDVGVYYCVQATQFPIT |
| | | | FGQGTRLEIKGGGSSRSSSSGGGGSGGGGEVQLVQSGAEVKKPGESLKIS |
| | | | CKGSGYSFTSYWIGWVRQMPGKGLEWMGIIYPGDSDTRYSPSFQGQVTI |
| | | | SADKSISTAYLQWSSLKASDTAMYYCARLGDYYNYYGMDVWGQGTTV |
| | | | TVSS |
| 431 | scFv_59 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GGCCATCCAGTTCCCCATCACCTTCGGCCAGGGCACCCGGCTGGAGAT |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCACCACCGCCTACCTGCAGTGGAGCAGCCTGAAGGCCAGCG |
| | | | ACACCGCCATGTACTACTGCGCCCGGCACCCCAGCTACGGCAGCGGCA |
| | | | GCCCCAACTTCGACTACTGGGGCCAGGGCACCCTGGTGACCGTGAGC |
| | | | AGC |
| 476 | scFv_59 | AA | DIVMTQTPLSSPVTLGQPASISCRSSQSLVHSDGNTYLSWLQQRPGQPPRL |
| | | | LIYKISNRFSGVPDRFSGSGAGTDFTLKISRVEAEDVGVYYCMQAIQFPITF |
| | | | GQGTRLEIKGGGSSRSSSSGGGGSGGGGEVQLVQSGAEVKKPGESLKISC |
| | | | KGSGYSFTSYWIGWVRQMPGKGLEWMGIIYPGDSDTRYSPSFQGQVTIS |
| | | | ADKSITTAYLQWSSLKASDTAMYYCARHPSYGSGSPNFDYWGQGTLVTV |
| | | | SS |
| 432 | scFv_60 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGTACAG |
| | | | CGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCACCCGGCAGGTGTTCGAGCCCGCCCTGGAGACCGACTTCGTG |
| | | | GGCGGCGGCGTGGGCTACACCTACAAGTGCGTGGTGGAGCAGATCGC |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCACCACCGCCTACCTGCAGTGGAGCAGCCTGAAGGCCAGCG |
| | | | ACACCGCCATGTACTACTGCGCCCGGCACCCCAGCTACGGCAGCGGCA |
| | | | GCCCCAACTTCGACTACTGGGGCCAGGGCACCCTGGTGACCGTGAGC |
| | | | AGC |
| 477 | scFv_60 | AA | DIVMTQTPLSSPVTLGQPASISCRSSQSLVYSDGNTYLSWLQQRPGQPPRL |
| | | | LIYKISNRFSGVPDRFSGSGAGTDFTLKISTRQVFEPALETDFVGGGVGYT |
| | | | YKCVVEQIAKGGGSSRSSSSGGGGSGGGGEVQLVQSGAEVKKPGESLKIS |
| | | | CKGSGYSFTSYWIGWVRQMPGKGLEWMGIIYPGDSDTRYSPSFQGQVTI |
| | | | SADKSITTAYLQWSSLKASDTAMYYCARHPSYGSGSPNFDYWGQGTLVT |
| | | | VSS |
| 433 | scFv_61 | DNA | GACGTGGTGATGACCCAGAGCCCCCTGAGCCTGCCCGTGACCCTGGG |
| | | | CCAGCCCGCCAGCATCAGCTGCCGGAGCAGCCAGAGCCTGGTGTACA |
| | | | GCGACGGCAACGCCTACCTGAACTGGTTCCAGCAGCGGCCCGGCCAG |
| | | | AGCCCCCGGCGGCTGATCTACAAGGTGAGCAACTGGGACAGCGGCGT |
| | | | GCCCGACCGGTTCAGCGGCAGCGGCAGCGGCACCGACTTCACCCTGA |
| | | | AGATCCGGCGGGTGGAGGCCGAGGACGTGGGCGCCTACTACTGCATG |
| | | | CAGGGCATCCACTGCACCTTCGGCCCCGGCACCAAGGTGCACATCAAG |
| | | | GGCGGCGGCAGCAGCCGGAGCAGCAGCAGCGGCGGCGGCGGCAGCG |
| | | | GCGGCGGCGGCGAGGTGCAGCTGGTGCAGAGCGGCGCCGAGGTGAA |
| | | | GAAGCCCGGCGAGAGCCTGAAGATCAGCTGCAAGGGCAGCGGCTACA |
| | | | GCTTCACCAGCTACTGGATCGGCTGGGTGCGGCAGATGCCCGGCAAGG |
| | | | GCCTGGAGTGGATGGGCATCATCTACCCCGGCGACAGCGACACCCGGT |
| | | | ACAGCCCCAGCTTCCAGGGCCAGGTGACCATCAGCGCCGACAAGAGC |
| | | | ATCAGCACCGCCTACCTGCAGTGGAGCAGCCTGAAGGCCAGCGACAC |
| | | | CGCCATGTACTACTGCGCCCGGCACGGCGACTACAGCAACATCGACGC |
| | | | CTTCGACATCTGGGGCCAGGGCACCATGGTGACCGTGAGCAGC |
| 478 | scFv_61 | AA | DVVMTQSPLSLPVTLGQPASISCRSSQSLVYSDGNAYLNWFQQRPGQSPR |
| | | | RLIYKVSNWDSGVPDRFSGSGSGTDFTLKIRRVEAEDVGAYYCMQGIHCT |
| | | | FGPGTKVHIKGGGSSRSSSSGGGGSGGGGEVQLVQSGAEVKKPGESLKIS |
| | | | CKGSGYSFTSYWIGWVRQMPGKGLEWMGIIYPGDSDTRYSPSFQGQVTI |
| | | | SADKSISTAYLQWSSLKASDTAMYYCARHGDYSNIDAFDIWGQGTMVTV |
| | | | SS |
| 434 | scFv_62 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTTCCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GCTGACCCAGTTCCCCCTGACCTTCGGCGGCGGCACCAAGGTGGAGAT |
| | | | CCGGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCAGCACCGCCTACCTGCAGTGGAGCAGCCTGAAGGCCAGCG |
| | | | ACACCGCCATGTACTACTGCGCCCGGACCGGCACCACCGACTACTACA |
| | | | GCGGCATGGACGTGTGGGGCCAGGGCACCACCGTGACCGTGAGCAGC |
| 479 | scFv_62 | AA | DIVMTQTPLSSPVTLGQPASISCRSSQSLVHSDGNTFLSWLQQRPGQPPRL |
| | | | LIYKISNRFSGVPDRFSGSGAGTDFTLKISRVEAEDVGVYYCMQLTQFPLT |
| | | | FGGGTKVEIRGGGSSRSSSSGGGGSGGGGEVQLVQSGAEVKKPGESLKIS |
| | | | CKGSGYSFTSYWIGWVRQMPGKGLEWMGIIYPGDSDTRYSPSFQGQVTI |
| | | | SADKSISTAYLQWSSLKASDTAMYYCARTGTTDYYSGMDVWGQGTTVT |
| | | | VSS |
| 435 | scFv_63 | DNA | GACGTGGTGATGACCCAGAGCCCCCTGAGCCTGCCCGTGACCCTGGG |
| | | | CCAGCCCGCCAGCATCAGCTGCCGGAGCAGCCAGAGCCTGGTGTACA |
| | | | GCGACGGCAACACCTACCTGAACTGGTTCCAGCAGCGGCCCGGCCAG |
| | | | AGCCCCCGGCGGCTGATCTACAAGGTGAGCAACTGGGACAGCGGCGT |
| | | | GCCCGACCGGTTCAGCGGCAGCGGCAGCGGCACCGACTTCACCCTGA |
| | | | AGATCAGCCGGGTGGAGGCCGAGGACGTGGGCGTGTACTACTGCATG |
| | | | CAGGGCACCCACTGGCCCAGCATCACCTTCGGCCAGGGCACCCGGCT |
| | | | GGAGATCAAGGGCGGCGGCAGCAGCCGGAGCAGCAGCAGCGGCGGC |
| | | | GGCGGCAGCGGCGGCGGCGGCGAGGTGCAGCTGGTGCAGAGCGGCG |
| | | | CCGAGGTGAAGAAGCCCGGCGAGAGCCTGAAGATCAGCTGCAAGGGC |
| | | | AGCGGCTACAGCTTCACCAGCTACTGGATCGGCTGGGTGCGGCAGATG |
| | | | CCCGGCAAGGGCCTGGAGTGGATGGGCATCATCTACCCCGGCGACAGC |
| | | | GACACCCGGTACAGCCCCAGCTTCCAGGGCCAGGTGACCATCAGCGC |
| | | | CGACAAGAGCATCAGCACCGCCTACCTGCAGTGGAGCAGCCTGAAGG |
| | | | CCAGCGACACCGCCATGTACTACTGCGCCCGGCACGGCGACTTCTGGA |
| | | | GCGGCTACTACAGCTTCGACTACTGGGGCCAGGGCACCCTGGTGACCG |
| | | | TGAGCAGC |
| 480 | scFv_63 | AA | DVVMTQSPLSLPVTLGQPASISCRSSQSLVYSDGNTYLNWFQQRPGQSPR |
| | | | RLIYKVSNWDSGVPDRFSGSGSGTDFTLKISRVEAEDVGVYYCMQGTHW |
| | | | PSITFGQGTRLEIKGGGSSRSSSSGGGGSGGGGEVQLVQSGAEVKKPGESL |
| | | | KISCKGSGYSFTSYWIGWVRQMPGKGLEWMGIIYPGDSDTRYSPSFQGQ |
| | | | VTISADKSISTAYLQWSSLKASDTAMYYCARHGDFWSGYYSFDYWGQGT |
| | | | LVTVSS |
| 436 | scFv_64 | DNA | GACATCGTGATGACCCAGACCCCCCACAGCAGCCCCGTGACCCTGGGC |
| | | | CAGCCCGCCAGCATCAGCTGCCGGAGCAGCCAGAGCCTGGTGAGCCG |
| | | | GGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCAACCGGTTCAGCGGCAGCGGCGCCGGCACCGACTTCACCCTGAAG |
| | | | ATCAGCCGGGTGAAGGCCGAGGACGTGGGCGTGTACTACTGCATGCA |
| | | | GGCCACCCAGTTCCCCCTGACCTTCGGCCAGGGCACCCGGCTGGAGAT |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCAGCACCGCCTACCTGCAGTGGAGCAGCCTGAAGGCCAGCG |
| | | | ACACCGCCATGTACTACTGCGCCCGGCAGGGCGACTTCTGGAGCGGCT |
| | | | ACGGCGGCATGGACGTGTGGGGCCAGGGCACCACCGTGACCGTGAGC |
| | | | AGC |
| 481 | scFv_64 | AA | DIVMTQTPHSSPVTLGQPASISCRSSQSLVSRDGNTYLSWLQQRPGQPPRL |
| | | | LIYKISNRFSGVPNRFSGSGAGTDFTLKISRVKAEDVGVYYCMQATQFPLT |
| | | | FGQGTRLEIKGGGSSRSSSSGGGGSGGGGEVQLVQSGAEVKKPGESLKIS |
| | | | CKGSGYSFTSYWIGWVRQMPGKGLEWMGIIYPGDSDTRYSPSFQGQVTI |
| | | | SADKSISTAYLQWSSLKASDTAMYYCARQGDFWSGYGGMDVWGQGTT |
| | | | VTVSS |
| 437 | scFv_65 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGAGCCG |
| | | | GGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGTGGAGGACGTGGGCGTGTACTACTGCATGCA |
| | | | GGCCACCCAGTTCCCCATCACCTTCGGCCAGGGCACCCGGCTGGAGAT |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCGGC |
| | | | TACAGCTTCACCAGCTACTGGATCGGCTGGGTGCGGCAGATGCCCGGC |
| | | | AAGGGCCTGGAGTGGATGGGCATCATCTACCCCGGCGACAGCGACACC |
| | | | AAGTACAGCCCCAGCTTCCAGGGCCAGGTGACCATCAGCGCCGACAA |
| | | | GAGCATCAGCACCGCCTACCTGCAGTGGAGCAGCCTGAAGGCCAGCG |
| | | | ACACCGCCATGTACTACTGCGCCCGGCAGGGCGACTTCTGGAGCGGCA |
| | | | ACGGCGGCATGGACGTGTGGGGCCAGGGCACCACCGTGACCGTGAGC |
| | | | AGC |
| 482 | scFv_65 | AA | DIVMTQTPLSSPVTLGQPASISCRSSQSLVSRDGNTYLSWLQQRPGQPPRL |
| | | | LIYKISNRFSGVPDRFSGSGAGTDFTLKISRVEVEDVGVYYCMQATQFPIT |
| | | | FGQGTRLEIKGGGSSRSSSSGGGGSGGGGEVQLVQSGAEVKKPGESLKIS |
| | | | CKGSGYSFTSYWIGWVRQMPGKGLEWMGIIYPGDSDTKYSPSFQGQVTI |
| | | | SADKSISTAYLQWSSLKASDTAMYYCARQGDFWSGNGGMDVWGQGTT |
| | | | VTVSS |
| 438 | scFv_66 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTTCCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GGGCACCCAGTTCCCCCTGACCTTCGGCGGCGGCACCAAGGTGGAGA |
| | | | TCAAGGGCGGCGGCAGCAGCCGGAGCAGCAGCAGCGGCGGCGGCGG |
| | | | CAGCGGCGGCGGCGGCGAGGTGCAGCTGGTGCAGAGCGGCGCCGAG |
| | | | GTGAAGAAGCCCGGCGAGAGCCTGAAGATCAGCTGCAAGGGCAGCG |
| | | | GCTACAGCTTCACCAACTACTGGATCGGCTGGGTGCGGCAGATGCCCG |
| | | | GCAAGGGCCTGGAGTGGATGGGCATCATCTACCCCGGCGACAGCGAC |
| | | | ACCCGGTACAGCCCCAGCTTCCAGGGCCAGGTGACCATCAGCGCCGA |
| | | | CAAGAGCATCAGCACCGCCTACCTGCAGTGGAGCAGCCTGAAGGCCA |
| | | | GCGACACCGCCATGTACTACTGCGCCCGGCACGGCGACTACTACGCCA |
| | | | GCGAGAGCAGCGCCTTCGACATCTGGGGCCAGGGCACCATGGTGACC |
| | | | GTGAGCAGC |
| 483 | scFv_66 | AA | DIVMTQTPLSSPVTLGQPASISCRSSQSLVHSDGNTFLSWLQQRPGQPPRL |
| | | | LIYKISNRFSGVPDRFSGSGAGTDFTLKISRVEAEDVGVYYCMQGTQFPLT |
| | | | FGGGTKVEIKGGGSSRSSSSGGGGSGGGGEVQLVQSGAEVKKPGESLKIS |
| | | | CKGSGYSFTNYWIGWVRQMPGKGLEWMGIIYPGDSDTRYSPSFQGQVTI |
| | | | SADKSISTAYLQWSSLKASDTAMYYCARHGDYYASESSAFDIWGQGTMV |
| | | | TVSS |
| 439 | scFv_67 | DNA | GACATCGTGATGACCCAGAGCCCCCTGAGCAGCCCCGTGACCCTGGGC |
| | | | CAGCCCGCCAGCATCAGCTGCCGGTACAGCCAGAGCCTGGTGCACCG |
| | | | GGACGGCAACACCTACCTGAGCTGGCTGCAGCAGCGGCCCGGCCAGC |
| | | | CCCCCCGGCTGGTGATCCACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCTTCTACTACTGCATGCAG |
| | | | ACCACCCAGTTCCCCCTGACCTTCGGCGGCGGCACCAAGGTGGAGATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCAGCTACTGGATCGACTGGGTGCGGCAGATGCCCGGCA |
| | | | AGGGCCTGGAGTGGATGGGCAGCATCTACCCCGGCGACAGCGACACC |
| | | | CGGTACAGCCCCAGCTTCCAGGGCCAGGTGACCATCAGCGCCGACAA |
| | | | GAGCATCAGCACCGCCTACCTGCAGTGGAGCAGCCTGAAGGCCAGCG |
| | | | ACACCGCCATGTACTACTGCGCCCGGCAGGGCGCCAGCGGCTACTACT |
| | | | ACGGCATGGACGTGTGGGGCCAGGGCACCACCGTGACCGTGAGCAGC |
| 484 | scFv_67 | AA | DIVMTQSPLSSPVTLGQPASISCRYSQSLVHRDGNTYLSWLQQRPGQPPRL |
| | | | VIHKISNRFSGVPDRFSGSGAGTDFTLKISRVEAEDVGFYYCMQTTQFPLT |
| | | | FGGGTKVEIKGGGSSRSSSSGGGGSGGGGEVQLVQSGAEVKKPGESLKIS |
| | | | CKGSGYSFTSYWIDWVRQMPGKGLEWMGSIYPGDSDTRYSPSFQGQVTI |
| | | | SADKSISTAYLQWSSLKASDTAMYYCARQGASGYYYGMDVWGQGTTVT |
| | | | VSS |
| 440 | scFv_68 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GGCCACCCAGTTCCCCCTGACCTTCGGCGGCGGCACCAAGGTGGAGA |
| | | | TCAAGGGCGGCGGCAGCAGCCGGAGCAGCAGCAGCGGCGGCGGCGG |
| | | | CAGCGGCGGCGGCGGCCAGGTGCAGCTGGTGCAGAGCGGCGCCGAG |
| | | | GTGAAGAAGCCCGGCGCCAGCGTGAAGGTGAGCTGCACCGCCAGCGG |
| | | | CTACAGCTTCAGCAGCTACGGCATCAGCTGGGTGCGGCAGGCCCCCGG |
| | | | CCAGGGCCTGGAGTGGATGGGCTGGATCAGCGCCTACAACGGCCACA |
| | | | CCCGGTACGCCCAGAAGCTGCAGGGCCGGGTGACCATGACCAGCGAC |
| | | | ACCAGCACCAGCACCGCCTACATGGAGCTGCGGAGCCTGCGGAGCGA |
| | | | CGACACCGCCGTGTACTACTGCGCCCGGGCCAGCAGCTGGTACTTCGA |
| | | | CTGCTGGGGCCAGGGCACCCTGGTGACCGTGAGCAGC |
| 485 | scFv_68 | AA | DIVMTQTPLSSPVTLGQPASISCRSSQSLVHSDGNTYLSWLQQRPGQPPRL |
| | | | LIYKISNRFSGVPDRFSGSGAGTDFTLKISRVEAEDVGVYYCMQATQFPLT |
| | | | FGGGTKVEIKGGGSSRSSSSGGGGSGGGGQVQLVQSGAEVKKPGASVKV |
| | | | SCTASGYSFSSYGISWVRQAPGQGLEWMGWISAYNGHTRYAQKLQGRVT |
| | | | MTSDTSTSTAYMELRSLRSDDTAVYYCARASSWYFDCWGQGTLVTVSS |
| 441 | scFv_69 | DNA | GAGATCGTGATGACCCAGACCCCCCTGAGCAGCCCCGTGACCCTGGGC |
| | | | CAGCCCGCCAGCATCAGCTGCAAGAGCAGCCAGAGCCTGGTGCACAG |
| | | | CGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GACCACCTACTTCCCCCTGACCTTCGGCGGCGGCACCAAGGTGGAGAT |
| | | | CAAGGGCGGCGGCAGCAGCCGGAGCAGCAGCAGCGGCGGCGGCGGC |
| | | | AGCGGCGGCGGCGGCCAGGTGCAGCTGGTGCAGAGCGGCGCCGAGG |
| | | | TGAAGAAGCCCGGCGCCAGCGTGAAGGTGAGCTGCAAGGCCAGCGG |
| | | | CTACACCTTCACCAGCTACGGCATCAGCTGGGTGCGGCAGGCCCCCGG |
| | | | CCAGGGCCTGGAGTGGATGGGCTGGATCAACGCCTACAACGGCAACA |
| | | | CCAACTACGCCCAGAAGCTGCGGGGCCGGGTGACCATGACCACCGAC |
| | | | ACCTTCACCAGCACCGCCGACATGGAGCTGCGGAGCCTGCGGAGCGA |
| | | | CGACACCGCCGTGTACTACTGCGCCCGGGCCAGCACCGCCATGGGCGA |
| | | | CTACTGGGGCCAGGGCACCCTGGTGACCGTGAGCAGC |
| 486 | scFv_69 | AA | EIVMTQTPLSSPVTLGQPASISCKSSQSLVHSDGNTYLSWLQQRPGQPPRL |
| | | | LIYKISNRFSGVPDRFSGSGAGTDFTLKISRVEAEDVGVYYCMQTTYFPLT |
| | | | FGGGTKVEIKGGGSSRSSSSGGGGSGGGGQVQLVQSGAEVKKPGASVKV |
| | | | SCKASGYTFTSYGISWVRQAPGQGLEWMGWINAYNGNTNYAQKLRGRV |
| | | | TMTTDTFTSTADMELRSLRSDDTAVYYCARASTAMGDYWGQGTLVTVSS |
| 442 | scFv_70 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGTACAG |
| | | | CGACGGCAACACCTACCTGAGCTGGCTGCACCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCCTGTACTACTGCATGCAG |
| | | | ACCACCCAGTTCCCCCTGACCTTCGGCGGCGGCACCAAGGTGAAGATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCAACTACTGGATCGCCTGGGTGCGGCAGATGCCCGGCA |
| | | | AGGGCCTGGAGTGGATGGGCATCATCTTCCCCGGCGACAGCGACACCC |
| | | | GGTACAGCCCCGCCTTCCAGGGCCAGGTGACCATCAGCGCCGACAAG |
| | | | AGCATCAGCACCGCCTACCTGCACTGGAGCAGCCTGAAGGCCAGCGA |
| | | | CACCGCCATGTACTACTGCGCCCGGCACCGGGACTACACCAGCGGCGG |
| | | | CCCCGACGCCTTCGACGTGTGGGGCCAGGGCACCATGGTGACCGTGA |
| | | | GCAGC |
| 487 | scFv_70 | AA | DIVMTQTPLSSPVTLGQPASISCRSSQSLVYSDGNTYLSWLHQRPGQPPRL |
| | | | LIYKISNRFSGVPDRFSGSGAGTDFTLKISRVEAEDVGLYYCMQTTQFPLT |
| | | | FGGGTKVKIKGGGSSRSSSSGGGGSGGGGEVQLVQSGAEVKKPGESLKIS |
| | | | CKGSGYSFTNYWIAWVRQMPGKGLEWMGIIFPGDSDTRYSPAFQGQVTI |
| | | | SADKSISTAYLHWSSLKASDTAMYYCARHRDYTSGGPDAFDVWGQGTM |
| | | | VTVSS |
| 443 | scFv_71 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTACCTGAGCTGGCTGCAGCAGCGGCCCGGCCAGC |
| | | | CCCCCCGGCTGCTGATCTACAAGCTGAGCAACCGGTTCAGCGGCATCC |
| | | | CCGACAGCCGGCGGTTCGTGAGCGGCGAGGCCAGCGAGGGCGACGCC |
| | | | GTGGGCGGCACCGTGGACTACTTCTACACCTGCCTGAAGATGCAGATC |
| | | | GCCACCCAGTTCCCCCTGACCTTCGGCGGCGGCACCAAGGTGGAGATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCAGCTACTGGATCGCCTGGGTGCGGCAGATGCCCGGCA |
| | | | AGGGCCTGGAGTGGATGGGCATCATCTACCCCGGCGACAGCGACACCC |
| | | | GGTACAGCCCCAGCTTCCAGGGCCAGGTGACCATCAGCGCCGACAAG |
| | | | AGCATCAGCACCGCCTACCTGCAGTGGAGCAACCTGAAGGCCAGCGA |
| | | | CACCGCCATGTACTACTGCGCCCGGACCGGCAGCTACTACAACTACTG |
| | | | CGGCATGGACGTGTGGGGCCAGGGCACCACCGTGACCGTGAGCAGC |
| 488 | scFv_71 | AA | DIVMTQTPLSSPVTLGQPASISCRSSQSLVHSDGNTYLSWLQQRPGQPPRL |
| | | | LIYKLSNRFSGIPDSRRFVSGEASEGDAVGGTVDYFYTCLKMQIATQFPLT |
| | | | FGGGTKVEIKGGGSSRSSSSGGGGSGGGGEVQLVQSGAEVKKPGESLKIS |
| | | | CKGSGYSFTSYWIAWVRQMPGKGLEWMGIIYPGDSDTRYSPSFQGQVTIS |
| | | | ADKSISTAYLQWSNLKASDTAMYYCARTGSYYNYCGMDVWGQGTTVT |
| | | | VSS |
| 444 | scFv_72 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGTACCG |
| | | | GGACGGCAACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GGCCACCCAGTTCCCCCTGACCTTCGGCGGCGGCACCAAGGTGGAGA |
| | | | TCAAGGGCGGCGGCAGCAGCCGGAGCAGCAGCAGCGGCGGCGGCGG |
| | | | CAGCGGCGGCGGCGGCGAGGTGCAGCTGGTGCAGAGCGGCGCCGAG |
| | | | GTGAAGAAGCCCGGCGAGAGCCTGAAGATCAGCTGCAAGGGCAGCG |
| | | | GCTACAGCTTCACCAACTACTGGATCGCCTGGGTGCGGCAGATGCCCG |
| | | | GCAAGGGCCTGGAGTGGATGGGCATCATCTACCCCGGCGACAGCGAC |
| | | | ACCCGGTACAGCCCCAGCTTCCAGGGCCAGGTGACCATCAGCGCCGA |
| | | | CAAGAGCATCAGCACCGCCTACCTGCAGTGGAGCAGCCTGAAGGCCA |
| | | | GCGACACCGCCATGTACTACTGCACCCGGCAGGGCGACTACTACGACA |
| | | | GCAGCGGCCCCGACTACTGGGGCCAGGGCACCCTGGTGACCGTGAGC |
| | | | AGC |
| 489 | scFv_72 | AA | DIVMTQTPLSSPVTLGQPASISCRSSQSLVYRDGNTYLSWLQQRPGQPPRL |
| | | | LIYKISNRFSGVPDRFSGSGAGTDFTLKISRVEAEDVGVYYCMQATQFPLT |
| | | | FGGGTKVEIKGGGSSRSSSSGGGGSGGGGEVQLVQSGAEVKKPGESLKIS |
| | | | CKGSGYSFTNYWIAWVRQMPGKGLEWMGIIYPGDSDTRYSPSFQGQVTI |
| | | | SADKSISTAYLQWSSLKASDTAMYYCTRQGDYYDSSGPDYWGQGTLVT |
| | | | VSS |
| 445 | scFv_73 | DNA | AAGATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTACCTGAGCTGGTTCCAGCAGCGGCCCGGCCAGC |
| | | | CCCCCCGGCTGCTGATCAACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GGCCACCCAGTTCCCCCTGACCTTCGGCGGCGGCACCAAGGTGGAGA |
| | | | TCGGCGGCGGCAGCAGCCGGAGCAGCAGCAGCGGCGGCGGCGGCAG |
| | | | CGGCGGCGGCGGCCAGGTGCAGCTGGTGGAGAGCGCCGGCGGCGTG |
| | | | GTGCAGCCCGGCCGGAGCCTGCGGCTGAGCTGCGCCGCCAGCGGCTT |
| | | | CACCTTCAGCAGCTACGGCATGCACTGGGTGCGGCAGGCCCCCGGCA |
| | | | AGGGCCTGGAGTGGGTGGCCGTGATCTGGCACGACGGCAGCAAGAAG |
| | | | TACTACGAGGACAGCGTGAAGGGCCGGTTCACCATCAGCCGGGACAA |
| | | | CAGCAAGAACACCCTGTACCTGCAGATGAACAGCCTGCGGGCCGAGG |
| | | | ACACCGCCGTGTACTACTGCGCCCGGAACTGGTTCTTCGACTACTGGG |
| | | | GCCAGGGCACCCTGGTGACCGTGAGCAGC |
| 490 | scFv_73 | AA | KIVMTQTPLSSPVTLGQPASISCRSSQSLVHSDGNTYLSWFQQRPGQPPRL |
| | | | LINKISNRFSGVPDRFSGSGAGTDFTLKISRVEAEDVGVYYCMQATQFPLT |
| | | | FGGGTKVEIGGGSSRSSSSGGGGSGGGGQVQLVESAGGVVQPGRSLRLSC |
| | | | AASGFTFSSYGMHWVRQAPGKGLEWVAVIWHDGSKKYYEDSVKGRFTI |
| | | | SRDNSKNTLYLQMNSLRAEDTAVYYCARNWFFDYWGQGTLVTVSS |
| 446 | scFv_74 | DNA | AGCAGCGAGCTGACCCAGGACCCCGCCGTGAGCGTGGCCCTGGGCCA |
| | | | GACCGTGCGGATCACCTGCCAGGGCGACAGCCTGCGGAGCTACTACG |
| | | | CCAGCTGGTACCAGCAGAAGCCCGGCCAGGCCCCCGTGCTGGTGATCT |
| | | | ACGGCAAGAACAACCGGCCCAGCGGCATCCCCGCCCGGTTCAGCGGC |
| | | | AGCGACAGCGGCAACACCGCCAGCCTGACCATCACCGGCGCCCAGGC |
| | | | CGAGGACGAGGCCGACTACTACTGCAACAGCCGGGACAGCAGCGGCA |
| | | | ACCACGTGGTGTTCGGCGGCGGCACCAAGCTGACCGTGCTGGGCGGC |
| | | | GGCAGCAGCCGGAGCAGCAGCAGCGGCGGCGGCGGCAGCGGCGGCG |
| | | | GCGGCCAGGTGCAGCTGGTGGAGAGCGGCGGCGGCCTGGTGAAGCCC |
| | | | GGCGGCAGCCTGCGGCTGAGCTGCGCCGCCAGCGGCTTCACCTTCAG |
| | | | CGACTACTACATGAGCTGGATCCGGCAGGCCCCCGGCAAGGGCCTGGA |
| | | | GTGGGTGAGCTACATCAGCAGCAGCGGCACCACCATCTACTACGCCGA |
| | | | CAGCGTGAAGGGCCGGTTCACCATCAGCCGGGACAACGCCAAGAACA |
| | | | GCCTGTACCTGCAGATGAACAGCCTGCGGGCCGAGGACACCGCCGTG |
| | | | TACTACTGCGCCCGGGACCTGTACTACGGCGGCAACAGCTACTACTTC |
| | | | GACTACTGGGGCCAGGGCACCCTGGTGACCGTGAGCAGC |
| 491 | scFv_74 | AA | SSELTQDPAVSVALGQTVRITCQGDSLRSYYASWYQQKPGQAPVLVIYGK |
| | | | NNRPSGIPARFSGSDSGNTASLTITGAQAEDEADYYCNSRDSSGNHVVFG |
| | | | GGTKLTVLGGGSSRSSSSGGGGSGGGGQVQLVESGGGLVKPGGSLRLSC |
| | | | AASGFTFSDYYMSWIRQAPGKGLEWVSYISSSGTTIYYADSVKGRFTISR |
| | | | DNAKNSLYLQMNSLRAEDTAVYYCARDLYYGGNSYYFDYWGQGTLVTV |
| | | | SS |
| 447 | scFv_75 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTACCTGAGCTGGCTGCAGCAGCGGCCCGGCCAGC |
| | | | CCCCCCGGCTGCTGCTGTACAAGAACAGCAACCGGTTCAGCGGCGTG |
| | | | CCCGACCGGTTCAGCGGCAGCGGCGCCGGCACCGACTTCACCCTGAA |
| | | | GATCAGCCGGGTGGAGGCCGAGGACGTGGGCGTGTACTACTGCATGC |
| | | | AGGCCACCCAGTTCCCCCTGACCTTCGGCGGCGGCACCAAGGTGGAG |
| | | | ATCAAGGGCGGCGGCAGCAGCCGGAGCAGCAGCAGCGGCGGCGGCG |
| | | | GCAGCGGCGGCGGCGGCGAGGTGCAGCTGGTGCAGAGCGGCGCCGA |
| | | | GGTGAAGAAGCCCGGCGAGAGCCTGAAGATCAGCTGCAAGGGCAGC |
| | | | GGCTACAGCTTCACCAGCTACTGGGGCATCATCGGCATCTGGTACGTG |
| | | | CCCCGGGGCCAGGACATGAGCCCCGACGGCACCAAGCGGGGCTACCT |
| | | | GAGCGAGCCCTGGAGCATGTTCCAGGGCCAGGTGACCATCAGCGCCG |
| | | | ACAAGAGCATCCGGACCGCCTACCTGCAGTGGAGCAGCCTGAAGGCC |
| | | | AGCGACACCGCCATGTACTACTGCGCCCGGATCGGCGACCACTACCAC |
| | | | TACAACGGCATGGACGTGTGGGGCCAGGGCACCACCGTGACCGTGAG |
| | | | CAGC |
| 492 | scFv_75 | AA | DIVMTQTPLSSPVTLGQPASISCRSSQSLVHSDGNTYLSWLQQRPGQPPRL |
| | | | LLYKNSNRFSGVPDRFSGSGAGTDFTLKISRVEAEDVGVYYCMQATQFPL |
| | | | TFGGGTKVEIKGGGSSRSSSSGGGGSGGGGEVQLVQSGAEVKKPGESLKI |
| | | | SCKGSGYSFTSYWGIIGIWYVPRGQDMSPDGTKRGYLSEPWSMFQGQVT |
| | | | ISADKSIRTAYLQWSSLKASDTAMYYCARIGDHYHYNGMDVWGQGTTV |
| | | | TVSS |
| 448 | scFv_76 | DNA | GACATCGTGATGACCCAGACCCCCCCCAGCAGCCCCGTGACCCGGGG |
| | | | CCAGCCCGCCAGCATCAGCTGCCGGAGCAGCCAGAGCCTGGTGCACA |
| | | | GCGACGGCAAGACCTACCTGAGCTGGCTGCAGCAGCGGCCCGGCCAG |
| | | | CCCCCCCGGCTGCTGATCTACAAGATCAGCAACCGGTTCAGCGGCGTG |
| | | | CCCGGCCGGTTCAGCGGCAGCGGCGCCGGCACCGACTTCACCCTGAA |
| | | | GATCAGCCGGGTGGAGGCCGAGGACGTGGGCGTGTACTACTGCATGC |
| | | | AGGCCACCCAGTTCCCCCTGACCTTCGGCGGCGGCACCAAGGTGGAG |
| | | | ATCAAGGGCGGCGGCAGCAGCCGGAGCAGCAGCAGCGGCGGCGGCG |
| | | | GCAGCGGCGGCGGCGGCGAGGTGCAGCTGGTGCAGAGCGGCGCCGA |
| | | | GGTGAAGAAGCCCGGCGAGAGCCTGAAGATCAGCTGCAAGGGCAGC |
| | | | GGCTACAGCTTCAGCAGCTACTGGATCAACTGGGTGCGGCAGATGCCC |
| | | | GGCAAGGGCCTGGAGTGGATGGGCATCATCTACCCCGGCGACAGCGA |
| | | | CACCCGGTACAGCCCCAGCTTCCAGGGCCAGGTGACCATCAGCGCCG |
| | | | ACAAGAGCATCAGCACCGCCTACCTGCAGTGGCGGAGCCTGAAGGCC |
| | | | AGCGACACCGCCATCTACTACTGCGCCCGGGTGGGCGACTACTACAGC |
| | | | TACTACGGCATGGACGTGTGGGGCCAGGGCACCACCGTGACCGTGAG |
| | | | CAGC |
| 493 | scFv_76 | AA | DIVMTQTPPSSPVTRGQPASISCRSSQSLVHSDGKTYLSWLQQRPGQPPRL |
| | | | LIYKISNRFSGVPGRFSGSGAGTDFTLKISRVEAEDVGVYYCMQATQFPLT |
| | | | FGGGTKVEIKGGGSSRSSSSGGGGSGGGGEVQLVQSGAEVKKPGESLKIS |
| | | | CKGSGYSFSSYWINWVRQMPGKGLEWMGIIYPGDSDTRYSPSFQGQVTIS |
| | | | ADKSISTAYLQWRSLKASDTAIYYCARVGDYYSYYGMDVWGQGTTVTV |
| | | | SS |
| 449 | scFv_77 | DNA | GACATCGTGATGACCCAGACCCCCCTGAGCAGCCCCGTGACCCTGGGC |
| | | | CAGCCCGCCAGCATCAGCTGCCGGAGCCGGCAGAGCCTGCTGCACAG |
| | | | CGACGGCAACACCTACCTGAGCTGGCTGCAGCAGCGGCCCGGCCAGC |
| | | | CCCCCCGGCTGCTGATCTACAAGCTGAGCAACCGGGTGAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GAGCACCCAGTTCCCCCTGACCTTCGGCGGCGGCACCAAGGTGAAGA |
| | | | TCAAGGGCGGCGGCAGCAGCCGGAGCAGCAGCAGCGGCGGCGGCGG |
| | | | CAGCGGCGGCGGCGGCGAGGTGCAGCTGGTGCAGAGCGGCGCCGAG |
| | | | GTGAAGAAGCCCGGCGAGAGCCTGAAGATCAGCTGCAAGGGCAGCG |
| | | | GCTACAGCTTCACCAACTACTGGATCAACTGGGTGCGGCAGATGCCCG |
| | | | GCAAGGGCCTGGAGTGGATGGGCATCATCTACCCCGGCGACAGCGAC |
| | | | ACCCGGTACAGCCCCAGCTTCCAGGGCCAGGTGACCATCAGCGCCGA |
| | | | CAAGAGCATCAACACCGCCTACCTGCAGTGGAGCAGCCTGAAGGCCA |
| | | | GCGACACCGCCATGTACTACTGCGCCCGGCAGGGCGGCCACTACTACT |
| | | | ACAGCGGCATGGACGTGTGGGGCCAGGGCACCACCGTGACCGTGAGC |
| | | | AGC |
| 494 | scFv_77 | AA | DIVMTQTPLSSPVTLGQPASISCRSRQSLLHSDGNTYLSWLQQRPGQPPRL |
| | | | LIYKLSNRVSGVPDRFSGSGAGTDFTLKISRVEAEDVGVYYCMQSTQFPL |
| | | | TFGGGTKVKIKGGGSSRSSSSGGGGSGGGGEVQLVQSGAEVKKPGESLKI |
| | | | SCKGSGYSFTNYWINWVRQMPGKGLEWMGIIYPGDSDTRYSPSFQGQVT |
| | | | ISADKSINTAYLQWSSLKASDTAMYYCARQGGHYYYSGMDVWGQGTTV |
| | | | TVSS |
| 450 | scFv_78 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACCGGAACACCTACCTGAGCTGGCTGCAGCAGCGGCCCGGCCAGC |
| | | | CCCCCCGGCTGCTGATCTACAAGGTGAGCAACCGGTTCAGCGGCGTGC |
| | | | CCGAGCGGTTCAGCGGCAGCGGCACCGGCACCGACTTCACCCTGAAG |
| | | | ATCAGCCGGGTGGAGGCCGAGGACGTGGGCGTGTACTACTGCGTGCA |
| | | | GGAGACCCTGTTCCCCATCACCATCGGCCAGGGCACCCGGCTGGAGAT |
| | | | CAAGGGCGGCGGCAGCAGCCGGAGCAGCAGCAGCGGCGGCGGCGGC |
| | | | AGCGGCGGCGGCGGCGAGGTGCAGCTGGTGCAGAGCGGCGCCGAGG |
| | | | TGAAGAAGCCCGGCGAGAGCCTGAAGATCAGCTGCAAGGGCAGCCGG |
| | | | AACAGCTTCACCAACTACTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCAGCACCGCCTACCTGCAGTGGAGCAGCCTGAAGGCCAGCG |
| | | | ACACCGCCATGTACTACTGCGCCCGGACCGGCAGCTACAGCTACTACT |
| | | | ACGGCATGGACGTGTGGGGCCAGGGCACCACCGTGACCGTGAGCAGC |
| 495 | scFv_78 | AA | DIVMTQTPLSSPVTLGQPASISCRSSQSLVHSDRNTYLSWLQQRPGQPPRL |
| | | | LIYKVSNRFSGVPERFSGSGTGTDFTLKISRVEAEDVGVYYCVQETLFPITI |
| | | | GQGTRLEIKGGGSSRSSSSGGGGSGGGGEVQLVQSGAEVKKPGESLKISC |
| | | | KGSRNSFTNYWIGWVRQMPGKGLEWMGIIYPGDSDTRYSPSFQGQVTIS |
| | | | ADKSISTAYLQWSSLKASDTAMYYCARTGSYSYYYGMDVWGQGTTVTV |
| | | | SS |
| 451 | scFv_79 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTTCCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCATCTACTACTGCATGCAG |
| | | | GCCACCCAGTTCCCCCTGACCTTCGGCGGCGGCACCAAGGTGGAGATC |
| | | | AAGGGCGGCGGCAGCAGCCGGAGCAGCAGCAGCGGCGGCGGCGGCA |
| | | | GCGGCGGCGGCGGCGGCGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCAGCTACTGGATCGGCTGGGTGCGGCAGATGCCCGGCA |
| | | | AGGGCCTGGAGTGGATGGGCATCATCTACCCCGGCGACAGCGACACCC |
| | | | GGTACAGCCCCAGCTTCCAGGGCCAGGTGATCATCAGCGCCGACAAG |
| | | | AGCATCAGCACCGCCTACCTGCAGTGGAGCAGCCTGAAGGCCAGCGA |
| | | | CACCGCCATGTACTACTGCGCCCGGACCGGCGACTACTACAGCTACCA |
| | | | CGGCATGGACGTGTGGGGCCAGGGCACCACCGTGACCGTGAGCAGC |
| 496 | scFv_79 | AA | DIVMTQTPLSSPVTLGQPASISCRSSQSLVHSDGNTFLSWLQQRPGQPPRL |
| | | | LIYKISNRFSGVPDRFSGSGAGTDFTLKISRVEAEDVGIYYCMQATQFPLTF |
| | | | GGGTKVEIKGGGSSRSSSSGGGGSGGGGGVQLVQSGAEVKKPGESLKISC |
| | | | KGSGYSFTSYWIGWVRQMPGKGLEWMGIIYPGDSDTRYSPSFQGQVIISA |
| | | | DKSISTAYLQWSSLKASDTAMYYCARTGDYYSYHGMDVWGQGTTVTVS |
| | | | S |
| 452 | scFv_80 | DNA | GACATCGTGCTGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCCACACCTACCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGGCGCCGAGGACGTGGGCGTGTACTACTGCATGCAG |
| | | | GCCACCCAGTTCCCCCTGACCTTCGGCGGCGGCACCAAGGTGGAGATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGAGCGGCGAGAGCCTGAAGATCAGCTGCAAGGGCAGCGGC |
| | | | TACAGCTTCACCAACTTCTGGATCGGCTGGGTGCGGCAGATGCCCGGC |
| | | | AAGGGCCTGGAGTGGATGGGCATCATCTACCCCGGCGACAGCGACACC |
| | | | CGGTACAGCCCCAGCTTCCAGGGCCAGGTGACCATCAGCGCCGACAA |
| | | | GAGCATCAGCACCACCTACCTGCAGTGGAGCAGCCTGAAGGCCAGCG |
| | | | ACACCGCCATGTACTACTGCGCCCGGCACCCCCCCTACAGCGGCAGCT |
| | | | ACTACGCCGACGCCTTCGACATCTGGGGCCAGGGCACCATGGTGACCG |
| | | | TGAGCAGC |
| 497 | scFv_80 | AA | DIVLTQTPLSSPVTLGQPASISCRSSQSLVHSDGHTYLSWLQQRPGQPPRLL |
| | | | IYKISNRFSGVPDRFSGSGAGTDFTLKISRVGAEDVGVYYCMQATQFPLTF |
| | | | GGGTKVEIKGGGSSRSSSSGGGGSGGGGEVQLVQSGAEVKKSGESLKISC |
| | | | KGSGYSFTNFWIGWVRQMPGKGLEWMGIIYPGDSDTRYSPSFQGQVTIS |
| | | | ADKSISTTYLQWSSLKASDTAMYYCARHPPYSGSYYADAFDIWGQGTMV |
| | | | TVSS |
| 453 | scFv_81 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGAGCAG |
| | | | CGACGGCAACACCTACCTGAGCTGGCTGCAGCAGCGGCCCGGCCAGC |
| | | | CCCCCCGGCTGCTGATCTACAAGATCAGCAACCTGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCCTGTACTACTGCATGCAG |
| | | | GCCACCCAGTTCCCCCTGACCTTCGGCGGCGGCACCAAGGTGGAGATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCAGCTTCTGGATCGGCTGGGTGCGGCAGATGCCCGGCA |
| | | | AGGGCCTGGAGTGGATGGGCATCATCTACCCCGGCGACAGCGACACCC |
| | | | GGTACAGCCCCAGCTTCCGGGGCCTGGTGACCATCAGCGCCGACAAG |
| | | | AGCATCAGCACCGCCTACCTGCACTGGAGCAGCCTGAAGGCCAGCGA |
| | | | CACCGCCATCTTCTACTGCGCCCGGCACCCCCCCTACAGCGGCAGCTA |
| | | | CTACGCCGACGCCTTCGACATCTGGGGCCAGGGCACCATGGTGACCGT |
| | | | GAGCAGC |
| 498 | scFv_81 | AA | DIVMTQTPLSSPVTLGQPASISCRSSQSLVSSDGNTYLSWLQQRPGQPPRL |
| | | | LIYKISNLFSGVPDRFSGSGAGTDFTLKISRVEAEDVGLYYCMQATQFPLT |
| | | | FGGGTKVEIKGGGSSRSSSSGGGGSGGGGEVQLVQSGAEVKKPGESLKIS |
| | | | CKGSGYSFTSFWIGWVRQMPGKGLEWMGIIYPGDSDTRYSPSFRGLVTIS |
| | | | ADKSISTAYLHWSSLKASDTAIFYCARHPPYSGSYYADAFDIWGQGTMVT |
| | | | VSS |
| 454 | scFv_82 | DNA | GACCAGATCCCCGTGGCCATGAGCACCATCCAGAGCAGCTGCCCCCGG |
| | | | CTGAGCAGCAGCAGCCAGCCCAGCGTGCTGACCGTGCTGCACGGCAG |
| | | | CGACGGCAACACCTTCCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATCCAG |
| | | | GCCACCCAGTTCCCCCTGACCTTCGGCGGCGGCACCAAGGTGGAGATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCAGCTACTGGATCGGCTGGGTGCGGCAGATGCCCGGCA |
| | | | AGGGCCTGGAGTGGATGGGCATCATCTACCCCGGCGACAGCGACACCC |
| | | | GGTACAGCCCCAGCTTCCAGGGCCAGGTGACCATCAGCGCCGACAAG |
| | | | AGCATCAGCACCGCCTACCTGCAGTGGAGCAGCCTGAAGGCCAGCGA |
| | | | CACCGCCATGTACTACTGCGCCCGGACCGGCGACTACCACAACTACTA |
| | | | CGGCATGGACGTGTGGGGCCAGGGCACCACCGTGACCGTGAGCAGC |
| 499 | scFv_82 | AA | DQIPVAMSTIQSSCPRLSSSSQPSVLTVLHGSDGNTFLSWLQQRPGQPPRL |
| | | | LIYKISNRFSGVPDRFSGSGAGTDFTLKISRVEAEDVGVYYCIQATQFPLTF |
| | | | GGGTKVEIKGGGSSRSSSSGGGGSGGGGEVQLVQSGAEVKKPGESLKISC |
| | | | KGSGYSFTSYWIGWVRQMPGKGLEWMGIIYPGDSDTRYSPSFQGQVTIS |
| | | | ADKSISTAYLQWSSLKASDTAMYYCARTGDYHNYYGMDVWGQGTTVT |
| | | | VSS |
| 455 | scFv_83 | DNA | GACGTGGTGATGACCCAGAGCCCCCTGAGCCTGCCCGTGACCCTGGG |
| | | | CCAGCCCGCCAGCATCAGCTGCCGGAGCCGGCAGAGCCTGGTGTACA |
| | | | GCGACGGCAGCACCTACCTGAACTGGTTCCAGCAGCGGCCCGGCCAG |
| | | | AGCCCCCGGCGGCTGATCTACAAGGTGAGCAACTGGGACAGCGGCGT |
| | | | GCCCGACCGGTTCAGCGCCAGCGGCAGCGGCACCGACTTCACCCTGA |
| | | | AGATCAGCCGGGTGGAGGCCGAGGACGACGGCGTGTACCACTGCATG |
| | | | CAGGGCACCCACTGGCCCCTGCTGGCCTTCGGCGGCGGCACCAAGGT |
| | | | GGAGATCGGCGGCGGCAGCAGCCGGAGCAGCAGCAGCGGCGGCGGC |
| | | | GGCAGCGGCGGCGGCGGCGAGGTGCAGCTGGTGCAGAGCGGCGCCG |
| | | | AGGTGAAGAAGCCCGGCGAGAGCCTGAAGATCAGCTGCAAGGGCAG |
| | | | CGGCTACAGCTTCACCAGCTACTGGATCGGCTGGGTGCGGCAGATGCC |
| | | | CGGCAAGGGCCTGGAGTGGATGGGCATCATCTACCCCGCCGACAGCGA |
| | | | CACCCGGTACAGCCCCAGCTTCCAGGGCCAGGTGACCATCAGCGCCG |
| | | | ACAAGAGCCTGAACACCGCCTACCTGCAGTGGCGGAGCCTGAAGGCC |
| | | | AGCGACACCGCCATGTACTACTGCGCCCGGATCGGCGACTTCTACTACT |
| | | | ACAGCGGCATGGACGTGTGGGGCCAGGGCACCACCGTGACCGTGAGC |
| | | | AGC |
| 500 | scFv_83 | AA | DVVMTQSPLSLPVTLGQPASISCRSRQSLVYSDGSTYLNWFQQRPGQSPR |
| | | | RLIYKVSNWDSGVPDRFSASGSGTDFTLKISRVEAEDDGVYHCMQGTHW |
| | | | PLLAFGGGTKVEIGGGSSRSSSSGGGGSGGGGEVQLVQSGAEVKKPGESL |
| | | | KISCKGSGYSFTSYWIGWVRQMPGKGLEWMGIIYPADSDTRYSPSFQGQ |
| | | | VTISADKSLNTAYLQWRSLKASDTAMYYCARIGDFYYYSGMDVWGQGT |
| | | | TVTVSS |
| 456 | scFv_84 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCAACACCTTCCTGAGCTGGCTGCAGCAGCGGCCCGGCCAGC |
| | | | CCCCCCGGCTGCTGATCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCGTGTACTACTGCATGCA |
| | | | GGCCACCCAGTTCCCCCTGACCTTCGGCGGCGGCACCAAGGTGGAGA |
| | | | TCAAGGGCGGCGGCAGCAGCCGGAGCAGCAGCAGCGGCGGCGGCGG |
| | | | CAGCGGCGGCGGCGGCGAGGTGCAGCTGGTGCAGAGCGGCGCCGAG |
| | | | GTGAAGAAGCCCGGCGAGAGCCTGAAGATCAGCTGCAAGGGCAGCG |
| | | | GCTACATCTTCACCAGCTACTGGATCGCCTGGGTGCGGCAGATGCCCG |
| | | | GCAAGGGCCTGGAGTGGATGGGCATCATCTACCCCGGCGACAGCGAC |
| | | | ACCCGGTACAGCCCCAGCTTCCAGGGCCAGATCACCATCAGCGCCGAC |
| | | | AAGAGCATCAGCACCGCCTACCTGCAGTGGAGCAGCCTGAAGGCCAG |
| | | | CGACACCGCCATGTACTACTGCGCCCGGGTGGGCACCACCAACTACTA |
| | | | CTACGGCATGGACGTGTGGGGCCAGGGCACCAGCGTGACCGTGAGCA |
| | | | GC |
| 501 | scFv_84 | AA | DIVMTQTPLSSPVTLGQPASISCRSSQSLVHSDGNTFLSWLQQRPGQPPRL |
| | | | LIYKISNRFSGVPDRFSGSGAGTDFTLKISRVEAEDVGVYYCMQATQFPLT |
| | | | FGGGTKVEIKGGGSSRSSSSGGGGSGGGGEVQLVQSGAEVKKPGESLKIS |
| | | | CKGSGYIFTSYWIAWVRQMPGKGLEWMGIIYPGDSDTRYSPSFQGQITIS |
| | | | ADKSISTAYLQWSSLKASDTAMYYCARVGTTNYYYGMDVWGQGTSVTV |
| | | | SS |
| 457 | scFv_85 | DNA | GACATCGTGATGACCCAGACCCCCCTGAGCAGCCCCGTGACCCTGGGC |
| | | | CAGCCCGCCAGCATCAGCTGCCGGAGCAGCCAGAGCCTGGTGCACAG |
| | | | CGACGGCCACACCTACCTGAGCTGGCTGCAGCAGCGGCCCGGCCAGC |
| | | | CCCCCCGGCTGCTGTTCTACAAGATCAGCAACCGGTTCAGCGGCGTGC |
| | | | CCGACCGGTTCAGCGGCAGCGGCGCCGGCACCGACTTCACCCTGAAG |
| | | | ATCAGCCGGGTGGAGGCCGAGGACGTGGGCATCTACTACTGCATGCAG |
| | | | GCCACCCAGTTCCCCCTGACCTTCGGCGGCGGCACCAAGGTGGACATC |
| | | | AAGGGCGGCGGCAGCAGCCGGAGCAGCAGCAGCGGCGGCGGCGGCA |
| | | | GCGGCGGCGGCGGCGAGGTGCAGCTGGTGCAGAGCGGCGCCGAGGT |
| | | | GAAGAAGCCCGGCGAGAGCCTGAAGATCAGCTGCAAGGGCAGCGGCT |
| | | | ACAGCTTCACCACCTACTGGATCGGCTGGGTGCGGCAGATGCCCGGCA |
| | | | AGGGCCTGGAGTGGATGGGCATCATCTACCCCGGCGACAGCGACACCC |
| | | | GGTACAGCCCCAGCTTCCAGGGCCAGGTGACCATCAGCGCCGACAAG |
| | | | AGCATCAGCACCGCCTACCTGCAGTGGAGCAGCCTGAAGGCCAGCGA |
| | | | CACCGCCATGTACTACTGCGCCCGGATCGGCGACTACTACAGCTACAG |
| | | | CGGCCTGGACGTGTGGGGCCAGGGCACCACCGTGACCGTGAGCAGC |
| 502 | scFv_85 | AA | DIVMTQTPLSSPVTLGQPASISCRSSQSLVHSDGHTYLSWLQQRPGQPPRL |
| | | | LFYKISNRFSGVPDRFSGSGAGTDFTLKISRVEAEDVGIYYCMQATQFPLT |
| | | | FGGGTKVDIKGGGSSRSSSSGGGGSGGGGEVQLVQSGAEVKKPGESLKIS |
| | | | CKGSGYSFTTYWIGWVRQMPGKGLEWMGIIYPGDSDTRYSPSFQGQVTI |
| | | | SADKSISTAYLQWSSLKASDTAMYYCARIGDYYSYSGLDVWGQGTTVTV |
| | | | SS |
| 458 | scFv_86 | DNA | CAGCCCGTGCTGACCCAGCCCACCAGCCTGAGCGCCAGCCCCGGCGC |
| | | | CAGCGCCCGGCTGACCTGCACCCTGCGGAGCGGCATCAACCTGGGCA |
| | | | GCTACCGGATCTTCTGGTACCAGCAGAAGCCCGAGAGCCCCCCCCGGT |
| | | | ACCTGCTGAGCTACTACAGCGACAGCCGGAAGCACCAGGGCAGCGGC |
| | | | GTGCCCAGCCGGTTCAGCGGCAGCAAGGACGCCAGCAGCAACGCCGG |
| | | | CATCCTGGTGATCAGCGGCCTGCAGAGCGAGGACGAGGCCGACTACTA |
| | | | CTGCATCTTCTGGCACAGCAGCGCCTGGGTGTTCGGCGGCGGCACCAA |
| | | | GCTGACCGTGGGCGGCGGCAGCAGCCGGAGCAGCAGCAGCGGCGGC |
| | | | GGCGGCAGCGGCGGCGGCGGCCAGGTGCAGCTGCAGCAGAGCGGCC |
| | | | CCGGCCTGATGAAGCCCAGCCAGACCCTGAGCCTGACCTGCGCCATCA |
| | | | GCGGCGACAGCGTGAGCAGCAACAGCGTGAGCTGGAACTGGATCCGG |
| | | | CAGAGCCCCAGCCGGGGCCTGGAGTGGCTGGGCCGGACCTACTACCG |
| | | | GTTCAAGTGGTTCAACGACTACGCCGTGAGCGTGAAGAGCCGGATCA |
| | | | CCATCAACCCCGACACCAGCAAGAACCAGTTCAGCCTGCGGCTGAAC |
| | | | AGCGTGACCCCCGAGGACACCGCCCTGTACTACTGCGCCCGGATCGAC |
| | | | ATCTGGAACGACGTGTTCGACTACTGGGGCCAGGGCACCCTGGTGACC |
| | | | GTGAGCAGC |
| 503 | scFv_86 | AA | QPVLTQPTSLSASPGASARLTCTLRSGINLGSYRIFWYQQKPESPPRYLLSY |
| | | | YSDSRKHQGSGVPSRFSGSKDASSNAGILVISGLQSEDEADYYCIFWHSSA |
| | | | WVFGGGTKLTVGGGSSRSSSSGGGGSGGGGQVQLQQSGPGLMKPSQTLS |
| | | | LTCAISGDSVSSNSVSWNWIRQSPSRGLEWLGRTYYRFKWFNDYAVSVKS |
| | | | RITINPDTSKNQFSLRLNSVTPEDTALYYCARIDIWNDVFDYWGQGTLVT |
| | | | VSS |

In some embodiments, the anti-CD20 amino acid sequence encoded by the second nucleic acid sequence encodes a polypeptide comprising one or more anti-CD20 CDRs capable of binding CD20. Various anti-CD20 amino acid sequences and their CDRs are known in the art and include, without limitation, those described in U.S. Pat. Nos. 5,736,137; 8,529,902; and 9,538,251. In some embodiments, the one or more anti-CD20 CDR amino acid sequences encoded by the second nucleic acid sequence are selected from the CDR amino acid sequences in Table 2B. In some embodiments, the one or more anti-CD20 CDR amino acid sequences encoded by the second nucleic acid sequence are selected from one or more CDR amino acid sequence of SEQ ID NOS: 504-526, 800-805, and 812-818 in Table 2B. In some embodiments, the one or more anti-CD20 CDR amino acid sequences encoded by the second nucleic acid sequence are selected from one or more functional variant of the CDR amino acid sequences of SEQ ID NOS: 504-526, 800-805, and 812-818 having at least about 70%, about 75%, about 80%, about 85%, about 90%, about 91%, about 92%, about 93%, about 94%, about 95%, about 96%, about 97%, about 98%, about 99% or more sequence identity to the respective amino acid sequence(s) of SEQ ID NOS: 504-526, 804-805, and 812-818. In some embodiments, the one or more anti-CD20 CDR amino acid sequences comprise at least three to six anti-CD20 CDR amino acid sequences. In some embodiments, the at least three to six anti-CDR amino acid sequence comprise, for a VL, a set of VL 1, 2, and 3 sequences from Table 2B, or a functional variant of the each having at least about 70%, about 75%, about 80%, about 85%, about 90%, about 91%, about 92%, about 93%, about 94%, about 95%, about 96%, about 97%, about 98%, about 99% or more sequence identity to the respective amino acid sequences of the set. In some embodiments, the at least three to six anti-CD20 CDR amino acid sequence comprise, for a VH, a set of VH 1, 2, and 3 sequences from Table 2B, or a functional variant of the each having at least about 70%, about 75%, about 80%, about 85%, about 90%, about 91%, about 92%, about 93%, about 94%, about 95%, about 96%, about 97%, about 98%, about 99% or more sequence identity to the respective amino acid sequences of the set.

The skilled artisan recognizes a CDR can be at about amino acid residues 24-34 (L1), 50-56 (L2) and 89-97 (L3) in the light chain variable domain and at about amino acid residues 31-35 (H1), 50-65 (H2) and 95-102 (H3 in the heavy chain variable domain as defined by Kabat et al. (Kabat et al. Sequences of Proteins of Immunological Interest, 5th Ed. Public Health Service, National Institutes of Health, Bethesda, Md. (1991)). Alternative definitions of the CDRs are also recognized in the art for example according to the scheme of Chothia (Chothia and Lesk (1987) J. Mol. Biol. 196: 901-917) in which case the CDRs are found at about amino acid residues 26-32 (L1), 50-52 (L2) and 91-96 (L3) in the light chain, and at about amino acid residues 26-32 (H1), 53-55 (H2) and 96-101 (H3) in the heavy chain. In some embodiments, the one or more anti-CD20 CDR are selected from an anti-CD20 VL or VH herein at the foregoing positions. In some embodiments, the one or more anti-CD20 CDR comprise about three to about six anti-CD20 CDR selected from an anti-CD20 VL or VH herein at the foregoing positions.

In some embodiments, the anti-CD20 CDRs comprise SEQ ID NOs: 800-805.

TABLE 2B
Exemplary CDRs of Anti-CD20 scFvs

| SEQ ID | | | |
|---|---|---|---|
| NO: | Name: | Type: | Sequence: |
| 504 | 2B8 VL CDR1 | AA | RASSSVSYIH |
| 505 | 2B8 VL CDR2 | AA | ATSNLAS |
| 506 | 2B8 VL CDR3 | AA | QQWTSNPPT |
| 507 | 2B8 VH CDR1 | AA | SYNMH |
| 508 | 2B8 VH CDR2 | AA | AIYPGNGDTSY |
| | | | NQKFKG |
| 509 | 2B8 VH CDR3 | AA | STYYGGDWYFNV |
| 510 | 2F2 VL CDR1 (human) | AA | RASQSVSSYLA |
| 511 | 2F2 VL CDR2 (human) | AA | DASNRAT |
| 512 | 2F2 VL CDR3 (human) | AA | QQRSNWPIT |
| 513 | 2F2 VH CDR1 (human) | AA | NDYAMH |
| 514 | 2F2 VH CDR2 (human) | AA | TISWNSGSIG |
| | | | YADSVKG |
| 515 | 2F2 VH CDR3 (human) | AA | DIQYGNYYYGMDV |
| 516 | VL CDR3 (human) | AA | QQRSDWPLT |
| 517 | VH CDR2 (human) | AA | TISWNSGTIG |
| | | | YADSVKG |
| 518 | VH CDR1 (human) | AA | YHAMH |
| 519 | VH CDR2 (human) | AA | IIGTGGVTYY |
| | | | ADSVKG |
| 520 | VH CDR3 (human) | AA | DYYGAGSFYD |
| | | | GLYGMDV |
| 812 | 7D8 VL CDR1 (human) | AA | RASQSVSSYLA |
| 813 | 7D8 VL CDR2 (human) | AA | DASNRAT |
| 814 | 7D8 VL CDR3 (human) | AA | QQRSNWPIT |
| 815 | 7D8 VH CDR1 (human) | AA | HDYAMH |
| 816 | 7D8 VH CDR2 (human) | AA | TISWNSGTIGY |
| | | | ADSVKG |
| 817 | 7D8 VH CDR3 (human) | AA | DIQYGNYYYGMDV |
| 521 | GA101 HVR-L1 | AA | RSSKSLLHSNGIT |
| | | | YLY |
| 522 | GA101 HVR-L2 | AA | QMSNLVS |
| 818 | GA101 HVR-L2 | AA | GMSNLVS |
| 523 | GA101 HVR-L3 | AA | AQNLELPYT |
| 524 | GA101 HVR-H1 | AA | GYAFSY |
| 525 | GA101 HVR-H2 | AA | FPGDGDTD |
| 526 | GA101 HVR-H3 | AA | NVFDGYWLVY |
| 800 | Leu16 VL CDR 1 | AA | RASSSVNYMD |
| 801 | Leu16 VL CDR 2 | AA | ATSNLAS |
| 802 | Leu16 VL CDR 3 | AA | QQWSFNPPT |
| 803 | Leu16 VH CDR 1 | AA | SYNMH |
| 804 | Leu16 VH CDR 2 | AA | AIYPGNGDTSYN |
| | | | QKFKG |
| 805 | Leu16 VH CDR 3 | AA | SNYYGSSYWFFDV |

In some embodiments, the anti-CD20 amino acid sequence encoded by a second nucleic acid sequence comprises an anti-CD20 light chain (VL). In some embodiments, the anti-CD20 light chain comprises the amino acid sequence of one of SEQ ID NOS: 527-576. See Table 2C. In some embodiments, the anti-CD20 light chain comprise the amino acid sequence of a functional variant of one of SEQ ID NOS: 527-576 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 527-576. In some embodiments, the anti-CD20 light chain comprises an amino acid sequence having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 537. In some embodiments, the anti-CD20 light chain comprises the amino acid sequence of SEQ ID NO: 537.

In some embodiments, the anti-CD20 amino acid sequence encoded by a second nucleic acid sequence comprises an anti-CD20 heavy chain (VH). In some embodiments, the anti-CD20 heavy chain comprises the amino acid sequence of one of SEQ ID NOS: 577-619. See Table 2C. In some embodiments, the anti-CD20 heavy chain comprises the amino acid sequence of a functional variant of one of SEQ ID NOS: 577-619 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 577-619. In some embodiments, the anti-CD20 heavy chain comprises an amino acid sequence having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 585. In some embodiments, the anti-CD20 heavy chain comprises the amino acid sequence of SEQ ID NO: 585.

In some embodiments, the anti-CD20 antigen binding domain comprises a light chain and a heavy chain. In some embodiments, the light chain comprises the amino acid sequence of one of SEQ ID NOS: 527-576. See Table 2C. In some embodiments, the light chain comprise the amino acid sequence of a functional variant of one of SEQ ID NOS: 527-576 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 527-576. In some embodiments, the heavy chain comprises the amino acid sequence of one of SEQ ID NOS: 577-619. See Table 2C. In some embodiments, the heavy chain comprises the amino acid sequence of a functional variant of one of SEQ ID NOS: 577-619 having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 577-619.

In some embodiments, the light chain precedes, that is, it is N-terminal to, the heavy chain. In some embodiments, the heavy chain precedes, that is, it is N-terminal to, the light chain. In some embodiments, an LH linker interposes between the light chain and the heavy chain. In some embodiments, the LH linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in length. In some embodiments, the linker is 19 amino acids in length. In some embodiments, the linker comprises the amino acid sequence GGGSSRSSSSGGGGSGGGG (SEQ ID NO: 413) or a function variant of the amino acid sequence GGGSSRSSSSGGGGSGGGG (SEQ ID NO: 413) having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 413. In some embodiments, the linker is 18 amino acids in length. In some embodiments, the linker comprises the amino acid sequence GSTSGGGSGGGSGGGGSS (SEQ ID NO: 806) or a functional variant of the amino acid sequence GSTSGGGSGGGSGGGGSS (SEQ ID NO: 806) having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 806. In some embodiments, the LH linker is encoded by a nucleic acid sequence comprising the sequence of one of SEQ ID NOS: 821-829, 831, or 833, or a functional variant of one of SEQ ID NOS: 821-829, 831, or 833 having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 821-829, 831, or 833. In some embodiments, the LH linker comprises an amino acid sequence comprising the sequence of SEQ ID NO: 830 or 832, or a function variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 830 or 832.

TABLE 2C
Exemplary Amino Acid Sequences of Anti-CD20 Light and Heavy Chains

| SEQ | | |
|---|---|---|
| ID | | |
| NO: | Name: | Sequence: |
| 527 | scFv_44 VL | MDFQVQIISFLLISASVIMSRGQIVLSQSPAILSASPGEKVTMTCRASSSVSYIHWF |
| | | QQKPGSSPKPWIYATSNLASGVPVRFSGSGSGTSYSLTISRVEAEDAATYYCQQW |
| | | TSNPPTFGGGTKLEIK |
| 577 | scFv_44 VH | MGWSLILLFLVAVATRVLSQVQLQQPGAELVKPGASVKMSCKASGYTFTSYNMH |
| | | WVKQTPGRGLEWIGAIYPGNGDTSYNQKFKGKATLTADKSSSTAYMQLSSLTSE |
| | | DSAVYYCARSTYYGGDWYFNVWGAGTTVTVSA |
| 528 | scFv_45 VL | EIVLTQSPATLSLSPGERATLSCRASQSVSSYLAWYQQKPGQAPRLLIYDASNRAT |
| | | GIPARFSGSGSGTDFTLTISSLEPEDFAVYYCQQRSNWPITFGQGTRLEIK |
| 578 | scFv_45 VH | EVQLVESGGGLVQPGRSLRLSCAASGFTFNDYAMHWVRQAPGKGLEWVSTISW |
| | | NSGSIGYADSVKGRFTISRDNAKKSLYLQMNSLRAEDTALYYCAKDIQYGNYYY |
| | | GMDVWGQGTTVTVSS |
| 529 | scFv_46 VL | EIVLTQSPATLSLSPGERATLSCRASQSVSSYLAWYQQKPGQAPRLLIYDASNRAT |
| | | GIPARFSGSGSGTDFTLTISSLEPEDFAVYYCQQRSNWPITFGQGTRLEIK |
| 579 | scFv_46 VH | EVQLVESGGGLVQPDRSLRLSCAASGFTFHDYAMHWVRQAPGKGLEWVSTISW |
| | | NSGTIGYADSVKGRFTISRDNAKNSLYLQMNSLRAEDTALYYCAKDIQYGNYYY |
| | | GMDVWGQGTTVTVSS |
| 530 | scFv_47 VL | EIVLTQSPATLSLSPGERATLSCRASQSVSSYLAWYQQKPGQAPRLLIYDASNRAT |
| | | GIPARFSGSGSGTDFTLTISSLEPEDFAVYYCQQRSDWPLTFGGGTKVEIK |
| 580 | scFv_47 VH | EVQLVQSGGGLVHPGGSLRLSCTGSGFTFSYHAMHWVRQAPGKGLEWVSIIGTG |
| | | GVTYYADSVKGRFTISRDNVKNSLYLQMNSLRAEDMAVYYCARDYYGAGSFY |
| | | DGLYGMDVWGQGTTVTVSS |
| 531 | scFv_48 VL | DIVMTQTPLSLPVTPGEPASISCRSSKSLLHSNGITYLYWYLQKPGQSPQLLIYQM |
| | | SNLVSGVPDRFSGSGSGTDFTLKISRVEAEDVGVYYCAQNLELPYTFGGGTKVEI |
| | | KRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| 581 | scFv_48 VH | QVQLVQSGAEVKKPGSSVKVSCKASGYAFSYSWINWVRQAPGQGLEWMGRIFP |
| | | GDGDTDYNGKFKGRVTITADKSTSTAYMELSSLRSEDTAVYYCARNVFDGYWL |
| | | VYWGQGTLVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFSCSVMHE |
| | | ALHNHYTQKSLSLSPG |
| 532 | scFv_49 VL | QIVLSQSPAILSASPGEKVTMTCRASSSVSYIHWFQQKPGSSPKPWIYATSNLASG |
| | | VPVRFSGSGSGTSYSLTISRVEAEDAATYYCQQWTSNPPTFGGGTKLEIK |
| 582 | scFv_49 VH | QVQLQQPGAELVKAGASVKMSCKASGYTFTSYNMHWVKQTPGRGLEWIGAIY |
| | | PGNGDTSYNQKFKGKATLTADKSSSTAYMQLSSLTSEDSAVYYCARSTYYGGDT |
| | | YFNVWGAGTTVTVSA |
| 533 | scFv_50.1 | QIVLSQSPAIITASPGEKVTMTCRASTSASYIHWFQQKPTSSPKPWIYATSNLASGV |
| | VL | PSRFSGSGSGTTYSMTISSLEAEDAATYYCQQWTSNPPTFGGGTKLEIK |
| 534 | scFv_50.2 | QIVLSQSPAIITASPGEKVTMTCRASTSVSYIHWFQQKPTSSPKPWIYATSNLASGV |
| | VL | PSRFSGSGSGTTYSMTISSLEAEDAATYYCQQWTSNPPTFGGGTKLEIK |
| 535 | scFv_50.3 | QIVLSQSPAIITASPGEKVTMTCRASTSVSYIHWFQQKPGSSPKPWIYATSNLASG |
| | VL | VPSRFSGSGSGTTYSMTISSLEAEDAATYYCQQWTSNPPTFGGGTKLEIK |
| 536 | scFv_50.4 | QIVLSQSPAIITASPGEKVTMTCRASSSVSYIHWFQQKPGSSPKPWIYATSNLASGV |
| | VL | PSRFSGSGSGTTYSMTISSLEAEDAATYYCQQWTSNPPTFGGGTKLEIK |
| 583 | scFv_50.1 | QVQLQQPGAELKKPGASVKVSCKASGYTFTSYNMHWVKQTPGRGLEWTGAIY |
| | VH | PGNGDTSYNQKFKGKTTLTADKSSSTAYMELSSLRSEDTAVYYCARSTYYGGDW |
| | | YFNVWGAGTTVTVSA |
| 584 | scFv_50.2 | QVQLQQPGAELKKPGASVKVSCKASGYTFTSYNMHWVKQTPGRGLEWIGAIYP |
| | VH | GNGDTSYNQKFKGKTTLTADKSSSTAYMELSSLRSEDTAVYYCARSTYYGGDW |
| | | YFNVWGAGTTVTVSA |
| 537 | scFv_51 VL | DIVLTQSPAILSASPGEKVTMTCRASSSVNYMDWYQKKPGSSPKPWIYATSNLAS |
| | | GVPARFSGSGSGTSYSLTISRVEAEDAATYYCQQWSFNPPTFGGGTKLEIK |
| 585 | scFv_51 VH | EVQLQQSGAELVKPGASVKMSCKASGYTFTSYNMHWVKQTPGQGLEWIGAIYP |
| | | GNGDTSYNQKFKGKATLTADKSSSTAYMQLSSLTSEDSADYYCARSNYYGSSYW |
| | | FFDVWGAGTTVTVSS |
| 538 | scFv_51.1 | DIVLTQSPAIITASPGEKVTMTCRASSSVNYMDWYQKKPGSSPKPWIYATSNLAS |
| | VL | GVPSRFSGSGSGTTYSMTISSLEAEDAATYYCQQWSFNPPTFGGGTKLEIK |
| 586 | scFv_51.1 | EVQLQQSGAELKKPGASVKVSCKASGYTFTSYNMHWVKQTPGQGLEWIGAIYP |
| | VH | GNGDTSYNQKFKGKTTLTADKSSSTAYMELSSLRSEDTAVYYCARSNYYGSSYW |
| | | FFDVWGTGTTVTVSS |
| 539 | scFv_52 VL | QIVLSQSPTILSASPGEKVTMTCRASSSVSYMDWYQQKPGSSPKPWIYATSNLAS |
| | | GVPARFSGSGSGTSYSLTISRVEAEDAATYYCQQWISNPPTFGAGTKLEL |
| 587 | scFv_52 VH | QVQLQQPGAELVKPGASVKMSCKTSGYTFTSYNVHWVKQTPGQGLEWIGAIYP |
| | | GNGDTSFNQKFKGKATLTADKSSSTVYMQLSSLTSEDSAVYYCARSNYYGSSYV |
| | | WFFDVWGAGTTGTGSS |
| 540 | scFv_53 VL | DIVMTQTPLSSPVTLGQPASISCRSSQSLVYSDGNTYLSWLQQRPGQPPRLLIYKIS |
| | | NRFSGVPDRFSGSGAGTDFTLKISTRQVFEPALETDFVGGGVGYTYKCVVEQIAK |
| 588 | scFv_53 VH | EVQLVQSGAEVKKPGESLKISCKGSGYSFTSYWIGWVRQMPGKGLEWMGIIYPG |
| | | DSDTRYRPSFQGQVTISADKSITTAYLQWSSLKASDTAMYYCARHPSYGSGSPNF |
| | | DYWGQGTLVTVSS |
| 541 | scFv_54 VL | EIVMTQSPATLSVSPGERATLSCRASQSVSSNLAWYQQKPGQAPRLLISGASTRAT |
| | | GIPARFSGSGSGTEFTLTISSLQSEDFAVYYCHQYNDWSLTFGGGTKVEIK |
| 589 | scFv_54 VH | SCKGSGYSFTSYWIGWVRQMPGKGLEWMGIIYPGDSDTRYSPSFQGQVTISADK |
| | | SISTAYLQWSSLKASDTAMYYCARMGDFWSGYYTRGMDVWGQGTTVTVSS |
| 542 | scFv_55 VL | DIVMTQTPLSSPVTLGQPASISCRSSQRLVHSDGHTYLSWLQQRPGQPPRLLISKV |
| | | SNRFSGVPDRFSGSGAGTDFTLKISRVEAEDVGIYYCMQATQFPLTFGGGTKVEI |
| | | K |
| 590 | scFv_55 VH | EVQLVQSGAEVKKPGESLKISCKGSGYSFTNYWIVWVRQMPGKGLEWMGIIYP |
| | | GDSDTRYSPSFQGQVTISADKSISTAYLQWSSLKASDTAMYYCARHGDYYYYYG |
| | | MDVWGQGTTVTVSS |
| 543 | scFv_56 VL | DIVMTQTPLSSPVTLGQPASISCRSSQSLVYSDGNTYLSWLQQRPGQPPRLLFYKI |
| | | SNRFSGVPDRFSGSGAGTDFTLKISRVEAEDVGVYYCVQATQFPLTFGGGTKVEI |
| | | K |
| 591 | scFv_56 VH | EVQLVQSGAEVKKPGESLKISCKGSGYSFTSYWIGWVRQMSGKGLEWMGIIYPG |
| | | DSDTRYSPSFQGQVTISADKSISTAYLQWSSLKASDTAIYYCARHPSYGSGSPNFD |
| | | YWGQGTLVTVSS |
| 544 | scFv_57 VL | DVVMTQSPLSLSVTLGQPASISCRSSQSLVYNDGDTYLNWFQQRPGQSPRLLIYK |
| | | VSNWDSGVPDRFSGSGSGTDFTLKVSRVEAEDVGVYYCMQGTHWPLTFGGGTK |
| | | VEIK |
| 592 | scFv_57 VH | EVQLVQSGAEVKKPGESLKISCKGSGYSFTNYWIGWVRQMPGKGLEWMGIIYP |
| | | GDSDTRYSPSFQGQVTISADKSISTAYLQWSSLKASDTAMYYCARQGDYYHYSG |
| | | MDVWGQGTTVTVSS |
| 545 | scFv_58 VL | DIVMTQTPLSSPVTLGQPASISCRSSQSLVHSDGKTYLSWLQQRPGQPPRLLIYKS |
| | | SNRFSGVPDRFSGSGAGTDFTLKISRVEAEDVGVYYCVQATQFPITFGQGTRLEIK |
| 593 | scFv_58 VH | EVQLVQSGAEVKKPGESLKISCKGSGYSFTSYWIGWVRQMPGKGLEWMGIIYPG |
| | | DSDTRYSPSFQGQVTISADKSISTAYLQWSSLKASDTAMYYCARLGDYYNYYGM |
| | | DVWGQGTTVTVSS |
| 546 | scFv_59 VL | DIVMTQTPLSSPVTLGQPASISCRSSQSLVHSDGNTYLSWLQQRPGQPPRLLIYKIS |
| | | NRFSGVPDRFSGSGAGTDFTLKISRVEAEDVGVYYCMQAIQFPITFGQGTRLEIK |
| 594 | scFv_59 VH | EVQLVQSGAEVKKPGESLKISCKGSGYSFTSYWIGWVRQMPGKGLEWMGIIYPG |
| | | DSDTRYSPSFQGQVTISADKSITTAYLQWSSLKASDTAMYYCARHPSYGSGSPNF |
| | | DYWGQGTLVTVSS |
| 547 | scFv_60 VL | DIVMTQTPLSSPVTLGQPASISCRSSQSLVYSDGNTYLSWLQQRPGQPPRLLIYKIS |
| | | NRFSGVPDRFSGSGAGTDFTLKISTRQVFEPALETDFVGGGVGYTYKCVVEQIAK |
| 595 | scFv_60 VH | EVQLVQSGAEVKKPGESLKISCKGSGYSFTSYWIGWVRQMPGKGLEWMGIIYPG |
| | | DSDTRYSPSFQGQVTISADKSITTAYLQWSSLKASDTAMYYCARHPSYGSGSPNF |
| | | DYWGQGTLVTVSS |
| 548 | scFv_61 VL | DVVMTQSPLSLPVTLGQPASISCRSSQSLVYSDGNAYLNWFQQRPGQSPRRLIYK |
| | | VSNWDSGVPDRFSGSGSGTDFTLKIRRVEAEDVGAYYCMQGIHCTFGPGTKVHI |
| | | K |
| 596 | scFv_61 VH | EVQLVQSGAEVKKPGESLKISCKGSGYSFTSYWIGWVRQMPGKGLEWMGIIYPG |
| | | DSDTRYSPSFQGQVTISADKSISTAYLQWSSLKASDTAMYYCARHGDYSNIDAFD |
| | | IWGQGTMVTVSS |
| 549 | scFv_62 VL | DIVMTQTPLSSPVTLGQPASISCRSSQSLVHSDGNTFLSWLQQRPGQPPRLLIYKIS |
| | | NRFSGVPDRFSGSGAGTDFTLKISRVEAEDVGVYYCMQLTQFPLTFGGGTKVEIR |
| 597 | scFv_62 VH | EVQLVQSGAEVKKPGESLKISCKGSGYSFTSYWIGWVRQMPGKGLEWMGIIYPG |
| | | DSDTRYSPSFQGQVTISADKSISTAYLQWSSLKASDTAMYYCARTGTTDYYSGM |
| | | DVWGQGTTVTVSS |
| 550 | scFv_63 VL | DVVMTQSPLSLPVTLGQPASISCRSSQSLVYSDGNTYLNWFQQRPGQSPRRLIYK |
| | | VSNWDSGVPDRFSGSGSGTDFTLKISRVEAEDVGVYYCMQGTHWPSITFGQGTR |
| | | LEIK |
| 598 | scFv_63 VH | EVQLVQSGAEVKKPGESLKISCKGSGYSFTSYWIGWVRQMPGKGLEWMGIIYPG |
| | | DSDTRYSPSFQGQVTISADKSISTAYLQWSSLKASDTAMYYCARHGDFWSGYYS |
| | | FDYWGQGTLVTVSS |
| 551 | scFv_64 VL | DIVMTQTPHSSPVTLGQPASISCRSSQSLVSRDGNTYLSWLQQRPGQPPRLLIYKI |
| | | SNRFSGVPNRFSGSGAGTDFTLKISRVKAEDVGVYYCMQATQFPLTFGQGTRLEI |
| | | K |
| 599 | scFv_64 VH | EVQLVQSGAEVKKPGESLKISCKGSGYSFTSYWIGWVRQMPGKGLEWMGIIYPG |
| | | DSDTRYSPSFQGQVTISADKSISTAYLQWSSLKASDTAMYYCARQGDFWSGYGG |
| | | MDVWGQGTTVTVSS |
| 552 | scFv_65 VL | DIVMTQTPLSSPVTLGQPASISCRSSQSLVSRDGNTYLSWLQQRPGQPPRLLIYKIS |
| | | NRFSGVPDRFSGSGAGTDFTLKISRVEVEDVGVYYCMQATQFPITFGQGTRLEIK |
| 600 | scFv_65 VH | EVQLVQSGAEVKKPGESLKISCKGSGYSFTSYWIGWVRQMPGKGLEWMGIIYPG |
| | | DSDTKYSPSFQGQVTISADKSISTAYLQWSSLKASDTAMYYCARQGDFWSGNGG |
| | | MDVWGQGTTVTVSS |
| 553 | scFv_66 VL | DIVMTQTPLSSPVTLGQPASISCRSSQSLVHSDGNTFLSWLQQRPGQPPRLLIYKIS |
| | | NRFSGVPDRFSGSGAGTDFTLKISRVEAEDVGVYYCMQGTQFPLTFGGGTKVEI |
| | | K |
| 601 | scFv_66 VH | EVQLVQSGAEVKKPGESLKISCKGSGYSFTNYWIGWVRQMPGKGLEWMGIIYP |
| | | GDSDTRYSPSFQGQVTISADKSISTAYLQWSSLKASDTAMYYCARHGDYYASESS |
| | | AFDIWGQGTMVTVSS |
| 554 | scFv_67 VL | DIVMTQSPLSSPVTLGQPASISCRYSQSLVHRDGNTYLSWLQQRPGQPPRLVIHKI |
| | | SNRFSGVPDRFSGSGAGTDFTLKISRVEAEDVGFYYCMQTTQFPLTFGGGTKVEI |
| | | K |
| 602 | scFv_67 VH | EVQLVQSGAEVKKPGESLKISCKGSGYSFTSYWIDWVRQMPGKGLEWMGSIYP |
| | | GDSDTRYSPSFQGQVTISADKSISTAYLQWSSLKASDTAMYYCARQGASGYYYG |
| | | MDVWGQGTTVTVSS |
| 555 | scFv_68 VL | DIVMTQTPLSSPVTLGQPASISCRSSQSLVHSDGNTYLSWLQQRPGQPPRLLIYKIS |
| | | NRFSGVPDRFSGSGAGTDFTLKISRVEAEDVGVYYCMQATQFPLTFGGGTKVEIK |
| 603 | scFv_68 VH | QVQLVQSGAEVKKPGASVKVSCTASGYSFSSYGISWVRQAPGQGLEWMGWISA |
| | | YNGHTRYAQKLQGRVTMTSDTSTSTAYMELRSLRSDDTAVYYCARASSWYFDC |
| | | WGQGTLVTVSS |
| 556 | scFv_69 VL | EIVMTQTPLSSPVTLGQPASISCKSSQSLVHSDGNTYLSWLQQRPGQPPRLLIYKIS |
| | | NRFSGVPDRFSGSGAGTDFTLKISRVEAEDVGVYYCMQTTYFPLTFGGGTKVEIK |
| 604 | scFv_69 VH | QVQLVQSGAEVKKPGASVKVSCKASGYTFTSYGISWVRQAPGQGLEWMGWIN |
| | | AYNGNTNYAQKLRGRVTMTTDTFTSTADMELRSLRSDDTAVYYCARASTAMGD |
| | | YWGQGTLVTVSS |
| 557 | scFv_70 VL | DIVMTQTPLSSPVTLGQPASISCRSSQSLVYSDGNTYLSWLHQRPGQPPRLLIYKIS |
| | | NRFSGVPDRFSGSGAGTDFTLKISRVEAEDVGLYYCMQTTQFPLTFGGGTKVKIK |
| 605 | scFv_70 VH | EVQLVQSGAEVKKPGESLKISCKGSGYSFTNYWIAWVRQMPGKGLEWMGIIFPG |
| | | DSDTRYSPAFQGQVTISADKSISTAYLHWSSLKASDTAMYYCARHRDYTSGGPD |
| | | AFDVWGQGTMVTVSS |
| 558 | scFv_71 VL | DIVMTQTPLSSPVTLGQPASISCRSSQSLVHSDGNTYLSWLQQRPGQPPRLLIYKL |
| | | SNRFSGIPDSRRFVSGEASEGDAVGGTVDYFYTCLKMQIATQFPLTFGGGTKVEI |
| | | K |
| 606 | scFv_71 VH | EVQLVQSGAEVKKPGESLKISCKGSGYSFTSYWIAWVRQMPGKGLEWMGIIYPG |
| | | DSDTRYSPSFQGQVTISADKSISTAYLQWSNLKASDTAMYYCARTGSYYNYCGM |
| | | DVWGQGTTVTVSS |
| 559 | scFv_72 VL | DIVMTQTPLSSPVTLGQPASISCRSSQSLVYRDGNTYLSWLQQRPGQPPRLLIYKI |
| | | SNRFSGVPDRFSGSGAGTDFTLKISRVEAEDVGVYYCMQATQFPLTFGGGTKVEI |
| | | K |
| 607 | scFv_72 VH | EVQLVQSGAEVKKPGESLKISCKGSGYSFTNYWIAWVRQMPGKGLEWMGIIYP |
| | | GDSDTRYSPSFQGQVTISADKSISTAYLQWSSLKASDTAMYYCTRQGDYYDSSGP |
| | | DYWGQGTLVTVSS |
| 560 | scFv_73 VL | KIVMTQTPLSSPVTLGQPASISCRSSQSLVHSDGNTYLSWFQQRPGQPPRLLINKIS |
| | | NRFSGVPDRFSGSGAGTDFTLKISRVEAEDVGVYYCMQATQFPLTFGGGTKVEI |
| 608 | scFv_73 VH | QVQLVESAGGVVQPGRSLRLSCAASGFTFSSYGMHWVRQAPGKGLEWVAVIWH |
| | | DGSKKYYEDSVKGRFTISRDNSKNTLYLQMNSLRAEDTAVYYCARNWFFDYWG |
| | | QGTLVTVSS |
| 561 | scFv_74 VL | SSELTQDPAVSVALGQTVRITCQGDSLRSYYASWYQQKPGQAPVLVIYGKNNRPS |
| | | GIPARFSGSDSGNTASLTITGAQAEDEADYYCNSRDSSGNHVVFGGGTKLTVL |
| 609 | scFv_74 VH | QVQLVESGGGLVKPGGSLRLSCAASGFTFSDYYMSWIRQAPGKGLEWVSYISSS |
| | | GTTIYYADSVKGRFTISRDNAKNSLYLQMNSLRAEDTAVYYCARDLYYGGNSYY |
| | | FDYWGQGTLVTVSS |
| 562 | scFv_75 VL | DIVMTQTPLSSPVTLGQPASISCRSSQSLVHSDGNTYLSWLQQRPGQPPRLLLYKN |
| | | SNRFSGVPDRFSGSGAGTDFTLKISRVEAEDVGVYYCMQATQFPLTFGGGTKVEI |
| | | K |
| 610 | scFv_75 VH | EVQLVQSGAEVKKPGESLKISCKGSGYSFTSYWGIIGIWYVPRGQDMSPDGTKR |
| | | GYLSEPWSMFQGQVTISADKSIRTAYLQWSSLKASDTAMYYCARIGDHYHYNG |
| | | MDVWGQGTTVTVSS |
| 563 | scFv_76 VL | DIVMTQTPPSSPVTRGQPASISCRSSQSLVHSDGKTYLSWLQQRPGQPPRLLIYKIS |
| | | NRFSGVPGRFSGSGAGTDFTLKISRVEAEDVGVYYCMQATQFPLTFGGGTKVEIK |
| 611 | scFv_76 VH | EVQLVQSGAEVKKPGESLKISCKGSGYSFSSYWINWVRQMPGKGLEWMGIIYPG |
| | | DSDTRYSPSFQGQVTISADKSISTAYLQWRSLKASDTAIYYCARVGDYYSYYGM |
| | | DVWGQGTTVTVSS |
| 564 | scFv_77 VL | DIVMTQTPLSSPVTLGQPASISCRSRQSLLHSDGNTYLSWLQQRPGQPPRLLIYKL |
| | | SNRVSGVPDRFSGSGAGTDFTLKISRVEAEDVGVYYCMQSTQFPLTFGGGTKVKI |
| | | K |
| 565 | scFv_77 VH | EVQLVQSGAEVKKPGESLKISCKGSGYSFTNYWINWVRQMPGKGLEWMGIIYP |
| | | GDSDTRYSPSFQGQVTISADKSINTAYLQWSSLKASDTAMYYCARQGGHYYYSG |
| | | MDVWGQGTTVTVSS |
| 566 | scFv_78 VL | DIVMTQTPLSSPVTLGQPASISCRSSQSLVHSDRNTYLSWLQQRPGQPPRLLIYKV |
| | | SNRFSGVPERFSGSGTGTDFTLKISRVEAEDVGVYYCVQETLFPITIGQGTRLEIK |
| 567 | scFv_78 VH | EVQLVQSGAEVKKPGESLKISCKGSRNSFTNYWIGWVRQMPGKGLEWMGIIYP |
| | | GDSDTRYSPSFQGQVTISADKSISTAYLQWSSLKASDTAMYYCARTGSYSYYYG |
| | | MDVWGQGTTVTVSS |
| 568 | scFv_79 VL | DIVMTQTPLSSPVTLGQPASISCRSSQSLVHSDGNTFLSWLQQRPGQPPRLLIYKIS |
| | | NRFSGVPDRFSGSGAGTDFTLKISRVEAEDVGIYYCMQATQFPLTFGGGTKVEIK |
| 612 | scFv_79 VH | GVQLVQSGAEVKKPGESLKISCKGSGYSFTSYWIGWVRQMPGKGLEWMGIIYP |
| | | GDSDTRYSPSFQGQVIISADKSISTAYLQWSSLKASDTAMYYCARTGDYYSYHG |
| | | MDVWGQGTTVTVSS |
| 569 | scFv_80 VL | DIVLTQTPLSSPVTLGQPASISCRSSQSLVHSDGHTYLSWLQQRPGQPPRLLIYKIS |
| | | NRFSGVPDRFSGSGAGTDFTLKISRVGAEDVGVYYCMQATQFPLTFGGGTKVEI |
| | | K |
| 613 | scFv_80 VH | EVQLVQSGAEVKKSGESLKISCKGSGYSFTNFWIGWVRQMPGKGLEWMGIIYPG |
| | | DSDTRYSPSFQGQVTISADKSISTTYLQWSSLKASDTAMYYCARHPPYSGSYYAD |
| | | AFDIWGQGTMVTVSS |
| 570 | scFv_81 VL | DIVMTQTPLSSPVTLGQPASISCRSSQSLVSSDGNTYLSWLQQRPGQPPRLLIYKIS |
| | | NLFSGVPDRFSGSGAGTDFTLKISRVEAEDVGLYYCMQATQFPLTFGGGTKVEIK |
| 614 | scFv_81 VH | EVQLVQSGAEVKKPGESLKISCKGSGYSFTSFWIGWVRQMPGKGLEWMGIIYPG |
| | | DSDTRYSPSFRGLVTISADKSISTAYLHWSSLKASDTAIFYCARHPPYSGSYYADA |
| | | FDIWGQGTMVTVSS |
| 571 | scFv_82 VL | DQIPVAMSTIQSSCPRLSSSSQPSVLTVLHGSDGNTFLSWLQQRPGQPPRLLIYKIS |
| | | NRFSGVPDRFSGSGAGTDFTLKISRVEAEDVGVYYCIQATQFPLTFGGGTKVEIK |
| 615 | scFv_82 VH | EVQLVQSGAEVKKPGESLKISCKGSGYSFTSYWIGWVRQMPGKGLEWMGIIYPG |
| | | DSDTRYSPSFQGQVTISADKSISTAYLQWSSLKASDTAMYYCARTGDYHNYYGM |
| | | DVWGQGTTVTVSS |
| 572 | scFv_83 VL | DVVMTQSPLSLPVTLGQPASISCRSRQSLVYSDGSTYLNWFQQRPGQSPRRLIYK |
| | | VSNWDSGVPDRFSASGSGTDFTLKISRVEAEDDGVYHCMQGTHWPLLAFGGGT |
| | | KVEI |
| 616 | scFv_83 VH | EVQLVQSGAEVKKPGESLKISCKGSGYSFTSYWIGWVRQMPGKGLEWMGIIYPA |
| | | DSDTRYSPSFQGQVTISADKSLNTAYLQWRSLKASDTAMYYCARIGDFYYYSGM |
| | | DVWGQGTTVTVSS |
| 573 | scFv_84 VL | DIVMTQTPLSSPVTLGQPASISCRSSQSLVHSDGNTFLSWLQQRPGQPPRLLIYKIS |
| | | NRFSGVPDRFSGSGAGTDFTLKISRVEAEDVGVYYCMQATQFPLTFGGGTKVEIK |
| 617 | scFv_84 VH | EVQLVQSGAEVKKPGESLKISCKGSGYIFTSYWIAWVRQMPGKGLEWMGIIYPG |
| | | DSDTRYSPSFQGQITISADKSISTAYLQWSSLKASDTAMYYCARVGTTNYYYGM |
| | | DVWGQGTSVTVSS |
| 574 | scFv_85 VL | DIVMTQTPLSSPVTLGQPASISCRSSQSLVHSDGHTYLSWLQQRPGQPPRLLFYKI |
| | | SNRFSGVPDRFSGSGAGTDFTLKISRVEAEDVGIYYCMQATQFPLTFGGGTKVDI |
| | | K |
| 618 | scFv_85 VH | EVQLVQSGAEVKKPGESLKISCKGSGYSFTTYWIGWVRQMPGKGLEWMGIIYPG |
| | | DSDTRYSPSFQGQVTISADKSISTAYLQWSSLKASDTAMYYCARIGDYYSYSGLD |
| | | VWGQGTTVTVSS |
| 575 | scFv_86 VL | QPVLTQPTSLSASPGASARLTCTLRSGINLGSYRIFWYQQKPESPPRYLLSYYSDS |
| | | RKHQGSGVPSRFSGSKDASSNAGILVISGLQSEDEADYYCIFWHSSAWVFGGGT |
| | | KLTV |
| 619 | scFv_86 VH | QVQLQQSGPGLMKPSQTLSLTCAISGDSVSSNSVSWNWIRQSPSRGLEWLGRTY |
| | | YRFKWFNDYAVSVKSRITINPDTSKNQFSLRLNSVTPEDTALYYCARIDIWNDVF |
| | | DYWGQGTLVTVSS |

Anti-CD19 and anti-CD20 KIR-CARs

The first fusion protein and the second fusion protein encoded by the one or more nucleic acid molecules described herein are in the form of KIR-CARs. KIR-CARs are described generally above.

In some embodiments, the first fusion protein (i.e., anti-CD19 KIR-CAR) encoded by a first nucleic acid sequence comprises an anti-CD19 antigen binding domain and a transmembrane domain. In some embodiments, the anti-CD19 antigen binding domain encoded by a first nucleic acid sequence further comprises one or more of a leader sequence, a tag, and a linker. In some embodiments, the leader precedes; that is, is N-terminal to, the anti-CD19 antigen binding domain. In some embodiments, the tag follows; that is, is C-terminal to, the anti-CD19 antigen binding domain. In some embodiments, the linker follows the anti-CD19 antigen binding domain. In some embodiments, the linker follows the tag, which follows the anti-CD19 antigen binding domain. In some embodiments, the transmembrane domain follows the anti-CD19 antigen binding domain. In some embodiments, the transmembrane domain follows the linker, which follows the tag, which follows the anti-CD19 antigen binding domain. In some embodiments, the first fusion protein encoded by a first nucleic acid sequence comprises, in the N-terminal to C-terminal direction, a leader sequence, an anti-CD19 antigen binding domain, a tag, a linker, and a transmembrane domain.

In some embodiments, the second fusion protein (i.e., anti-CD20 KIR-CAR) encoded by a second nucleic acid sequence comprises an anti-CD20 antigen binding domain and a transmembrane domain. In some embodiments, the anti-CD20 antigen binding domain encoded by a second nucleic acid sequence further comprises one or more of a leader sequence, a tag, and a linker. In some embodiments, the leader precedes; that is, is N-terminal to, the anti-CD20 antigen binding domain. In some embodiments, the tag follows; that is, is C-terminal to, the anti-CD20 antigen binding domain. In some embodiments, the linker follows the anti-CD20 antigen binding domain. In some embodiments, the linker follows the tag, which follows the anti-CD20 antigen binding domain. In some embodiments, the transmembrane domain follows the anti-CD20 antigen binding domain. In some embodiments, the transmembrane domain follows the linker, which follows the tag, which follows the anti-CD20 antigen binding domain. In some embodiments, the second fusion protein encoded by a second nucleic acid sequence comprises, in the N-terminal to C-terminal direction, a leader sequence, an anti-CD20 antigen binding domain, a tag, a linker, and a transmembrane domain.

In some embodiments, the first and second nucleic acid sequences are free of a nucleic acid sequence encoding a 41BB polypeptide, an anti-CD22 binding sequence, and/or a CD28 domain.

### Leader Sequence

As described above, the KIR-CARs of the present disclosure can include a leader sequence.

In some embodiments, the leader sequence can be a CD8α leader sequence. In some embodiments, the CD8α leader sequence comprises the amino acid sequence MALPVTALLLPLALLLHAARP ((SEQ ID NO: 357), Table 7). In some embodiments, the leader sequence comprises a functional variant of the amino acid sequence of SEQ ID NO: 357 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 357. In some embodiments, the leader sequence comprises the amino acid sequence of SEQ ID NOS: 674, 675, 676, or 754 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the amino acid sequence of 674, 675, 676, or 754. In some embodiments, the leader sequence comprises an IgK leader. In some embodiments, the IgK leader comprises the amino acid sequence METPAQLLFLLLLWLPDTTG ((SEQ ID NO: 754), Table 7). In some embodiments, the IgK leader comprises the amino acid sequence of a functional variant of the amino acid sequence of SEQ ID NO: 754 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 754.

In some embodiments, the leader sequence comprises a codon optimized nucleic acid sequence.

In some embodiments, leader sequence comprises a codon-optimized CD8α sequence that preserves the native amino acid sequence of the CD8α leader while substantially reducing nucleotide-level homology to other CD8α-derived regions within a multicistronic construct. As described in the Examples herein, initial dual-CAR and dual-KIR expression cassettes incorporating more than one copy of the CD8α signal peptide exhibited high levels of lentiviral template switching, resulting in undesired deletions and the generation of spurious single-binder constructs. Codon optimization of one copy of the CD8α signal peptide reduced shared nucleotide identity to below approximately 50%, thereby eliminating recombination hotspots while maintaining the encoded MALPVTALLLPLALLLHAARP (SEQ ID NO: 357) signal peptide sequence and its function in directing nascent polypeptides into the secretory pathway. The codon-optimized CD8α sequence additionally removes cryptic splice donor and acceptor motifs, improves GC balance, and enhances expression efficiency in mammalian cells, enabling stable and reliable production of full-length dual-KIR constructs in lentiviral systems. In some embodiments, the optimized CD8α comprises the nucleic acid sequence of SEQ ID NO: 699.

### Tags

As described above, the KIR-CARs of the present disclosure can also include a tag sequence.

In some embodiments, the tag is a Myc tag. In some embodiments, the Myc tag comprises the amino acid sequence EQKLISEEDL (SEQ ID NO: 746), Table 7). In some embodiments, the Myc tag comprises the amino acid sequence of a functional variant of the amino acid sequence of SEQ ID NO: 746 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 746. In some embodiments, the tag is a 6×His tag comprising HHHHHH (SEQ ID NO: 755). In some embodiments, the tag is a flag tag. In some embodiments, the flag tag comprises the amino acid sequence DYKDDDDK ((SEQ ID NO: 756), Table 7). In some embodiments, the flag tag comprises the amino acid sequence of a functional variant of the amino acid sequence of SEQ ID NO: 756 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 756. In some embodiments, the tag is a Strep tag II. In some embodiments, the Strep tag II comprises the amino acid sequence WSHPQFEK ((SEQ ID NO: 757), Table 7). In some embodiments, the Strep tag II comprises the amino acid sequence of a functional variant of the amino acid sequence of SEQ ID NO: 757 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 757. In some embodiments, the tag is an SBP tag. In some embodiments, the SBP tag comprises the amino acid sequence MDEKTTGWRGGHVVEGLAGELEQLRARLEHHPQGQREP ((SEQ ID NO: 758), Table 7). In some embodiments, the SBP tag comprises the amino acid sequence of a functional variant of the amino acid sequence of SEQ ID NO: 758 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 758. In some embodiments, the tag is an HA tag. In some embodiments, the HAtag comprises the amino acid sequence YPYDVPDYA (SEQ ID NO: 759), Table 7). In some embodiments, the HA tag comprises the amino acid sequence of a functional variant of the amino acid sequence of SEQ ID NO: 759 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 759.

In some embodiments, the linker comprises the amino acid sequence SGGGGSGGGGSS (SEQ ID NO: 260), Table 7). In some embodiments, the linker comprises the amino acid sequence of a functional variant of the amino acid sequence of SEQ ID NO: 260 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 260. In some embodiments, the linker is encoded by a nucleic acid sequence comprising the sequence of one of SEQ ID NOS: 821-829, 831, or 833, or a functional variant of one of SEQ ID NOS: 821-829, 831, or 833 having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 821-829, 831, or 833. In some embodiments, the linker comprises an amino acid sequence comprising the sequence of SEQ ID NO: 830 or 832, or a function variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 830 or 832.

### KIR Domains

As described herein, the first and second fusion proteins include domains from a KIR polypeptide.

In some embodiments, one or both of the first and second nucleic acid sequences described herein each comprise a nucleic acid sequence encoding one or more KIR2DS2 domain. In some embodiments, extracellular amino acid sequences encoded by one or both of the first and second nucleic acid sequences comprises at least a portion of an extracellular portion (e.g. a KIR stem domain) of a KIR polypeptide independently selected from KIR2DS2, KIR2DS1, KIR2DS3, KIR2DS4, KIR2DS5, KIR3DS1 or a functional fragment thereof.

In some embodiments, at least a portion of an extracellular portion (e.g. a KIR stem domain) of a KIR polypeptide is from a KIR2DS2 domain. In some embodiments, the KIR stem domain is a short stem domain. In some embodiments, the KIR stem domain is a long stem domain. A “short KIR stem domain” (also referred to as KIRShort) includes a minimal extracellular portion and lacks the full-length KIR2D stem sequence, resulting in a more compact receptor architecture. By contrast, a “long KIR stem domain” (also referred to as KIRLong) incorporates the full-length KIR2D stem region—for example, the extended stem domain from KIR2DS2.

In some embodiments, the first fusion protein can include a short KIR stem domain. In some embodiments, the second fusion protein can include a long KIR stem domain.

In some embodiments, the disclosure provides a codon-optimized KIRLong sequence (“KIRLong-CodOpt” in the Examples herein) comprising the full-length KIR2DS2 extracellular stem domain together with the native KIR2DS2 transmembrane and cytoplasmic signaling regions. As described in the Examples herein, the codon-optimized KIR-Long sequence is engineered to eliminate cryptic splice donor and acceptor motifs present in the non-optimized KIR-Long construct, including the splice event that previously generated a 561-base-pair truncated isoform, and to reduce nucleotide homology within the CD8α signal peptide and KIR2DS2 coding regions that otherwise promoted lentiviral template switching. As demonstrated in the application, codon optimization yields exclusive full-length expression of the KIR-Long fusion protein, prevents formation of unintended single-binder products, and significantly improves transgene expression and stability of lentiviral vectors. In some embodiments, the second fusion protein can include a long KIR stem domain that is encoded by a codon optimized nucleic acid sequence. In some embodiments, the codon optimized nucleic acid sequence comprises the nucleic acid sequence of SEQ ID NO: 707.

In some embodiments, the transmembrane domain of one or both of the first and second nucleic acid sequences comprises at least a portion of a transmembrane domain of a KIR polypeptide independently selected from KIR2DS2, KIR2DS1, KIR2DS3, KIR2DS4, KIR2DS5, KIR3DS1 or a functional variant thereof. In some embodiments, the transmembrane domain of one or both of the first and second nucleic acid sequences comprises at least a portion of a transmembrane domain of KIR2DS2. In some embodiments, the portion of a transmembrane domain of a KIR polypeptide comprises the amino acid sequence of SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753.

In some embodiments, the transmembrane domain of one or both of the first and second nucleic acid sequences comprises the KIR2DS2 transmembrane domain of SEQ ID NO: 742 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742.

In some embodiments, each of the transmembrane and cytoplasmic regions of the first and second fusion proteins comprise a KIR2DS2 polypeptide.

In some embodiments, the first nucleic acid sequence (encoding the anti-CD19 KIR-CAR) comprises an extracellular portion (e.g., stem) and transmembrane domain from KIR2DS2. In some embodiments, the extracellular portion (e.g., stem) and transmembrane domain from KIR2DS2 comprises the amino acid sequence of SEQ ID NO: 266.

In some embodiments, the second nucleic acid sequence (encoding the anti-CD20 KIR-CAR) comprises an extracellular portion (e.g., stem) and transmembrane domain from KIR2DS2. In some embodiments, the extracellular portion (e.g., stem) and transmembrane domain from KIR2DS2 comprises the amino acid sequence of SEQ ID NO: 705.

In some embodiments, one or both of the first nucleic acid sequence or the second nucleic acid sequence encode an intracellular domain. In some embodiments, the intracellular domain comprises an intracellular domain of a KIR polypeptide independently selected from KIR2DS2, KIR2DS1, KIR2DS3, KIR2DS4, KIR2DS5, KIR3DS1 or a functional variant thereof. See Table 3. In some embodiments, the intracellular domain comprises an intracellular domain of KIR2DS2.

In some embodiments, KIR2DS2, KIR2DS1, KIR2DS3, KIR2DS4, KIR2DS5, KIR3DS1 comprises the amino acid sequence of SEQ ID NOS: 668-673, respectively. In some embodiments, the KIR2DS2, KIR2DS1, KIR2DS3, KIR2DS4, KIR2DS5, KIR3DS1 comprises a functional variant of the amino acid sequence of SEQ ID NOS: 668-673, respectively, having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 668-673. See Table 3.

TABLE 3
KIR2DS Polypeptides

| SEQ | | |
|---|---|---|
| ID | | |
| NO: | Name: | Sequence: |
| 668 | KIR2DS2 | MSLMVVSMVCVGFFLLQGAWPHEGVHRKPSLLAHPGPLVKSEETVILQCW |
| | | SDVRFEHFLLHREGKYKDTLHLIGEHHDGVSKANFSIGPMMQDLAGTYRC |
| | | YGSVTHSPYQLSAPSDPLDIVITGLYEKPSLSAQPGPTVLAGESVTLSCSSRSS |
| | | YDMYHLSREGEAHERRFSAGPKVNGTFQADFPLGPATHGGTYRCFGSFRDS |
| | | PYEWSNSSDPLLVSVTGNPSNSWPSPTEPSSKTGNPRHLHVLIGTSVVKIPFTI |
| | | LLFFLLHRWCSNKKNAAVMDQEPAGNRTVNSEDSDEQDHQEVSYA |
| 669 | KIR2DS1 | MSLLVVSMACVGFFLLQGAWPHEGVHRKPSLLAHPGRLVKSEETVILQCWS |
| | | DVMFEHFLLHREGMFNDTLRLIGEHHDGVSKANFSISRMKQDLAGTYRCY |
| | | GSVTHSPYQLSAPSDPLDIVIIGLYEKPSLSAQPGPTVLAGENVTLSCSSRSSY |
| | | DMYHLSREGEAHERRLPAGTKVNGTFQANFPLGPATHGGTYRCFGSFRDSP |
| | | YEWSKSSDPLLVSVTGNPSNSWPSPTEPSSETGNPRHLHVLIGTSVVKIPFTIL |
| | | LFFLLHRWCSDKKNAAVMDQEPAGNRTVNSEDSDEQDHQEVSYA |
| 670 | KIR2DS3 | MSLMVISMACVGFFWLQGAWPHEGFRRKPSLLAHPGRLVKSEETVILQCW |
| | | SDVMFEHFLLHREGTFNDTLRLIGEHIDGVSKANFSIGRMRQDLAGTYRCY |
| | | GSVPHSPYQFSAPSDPLDIVITGLYEKPSLSAQPGPTVLAGESVTLSCSSWSS |
| | | YDMYHLSTEGEAHERRFSAGPKVNGTFQADFPLGPATQGGTYRCFGSFHDS |
| | | PYEWSKSSDPLLVSVTGNPSNSWPSPTEPSSKTGNPRHLHVLIGTSVVKLPFT |
| | | ILLFFLLHRWCSDKKNASVMDQGPAGNRTVNREDSDE |
| 671 | KIR2DS4 | MSLMVIIMACVGFFLLQGAWPQEGVHRKPSFLALPGHLVKSEETVILQCWS |
| | | DVMFEHFLLHREGKFNNTLHLIGEHHDGVSKANFSIGPMMPVLAGTYRCY |
| | | GSVPHSPYQLSAPSDPLDMVIIGLYEKPSLSAQPGPTVQAGENVTLSCSSRSS |
| | | YDMYHLSREGEAHERRLPAVRSINGTFQADFPLGPATHGGTNRCFGSFRDAP |
| | | YEWSNSSDPLLVSVTGNPSNSWPSPTEPSSKTGNPRHLHVLIGTSVVKIPFTIL |
| | | LFFLLHRWCSDKKNAAVMDQEPAGNRTVNSEDSDEQDHQEVSYA |
| 672 | KIR2DS5 | MSLMVISMACVAFFLLQGAWPHEGFRRKPSLLAHPGPLVKSEETVILQCWS |
| | | DVMFEHFLLHREGTFNHTLRLIGEHIDGVSKGNFSIGRMTQDLAGTYRCYG |
| | | SVTHSPYQLSAPSDPLDIVITGLYEKPSLSAQPGPTVLAGESVTLSCSSRSSYD |
| | | MYHLSREGEAHERRLPAGTKVNGTFQADFPLDPATHGGTYRCFGSFRDSPY |
| | | EWSKSSDPLLVSVTGNTSNSWPSPTEPSSKTGNPRHLHVLIGTSVVKLPFTIL |
| | | LFFLLHRWCSNKKNASVMDQGPAGNRTVNREDSDEQDHQEVSYA |
| 673 | KIR3DS1 | MLLMVVSMACVGLFLVQRAGPHMGGQDNPFLSAWPSAVVPRGGHVTLRC |
| | | HYRHRFNNFMLYKEDRIHVPIFHGRIFQEGFNMSPVTTAHAGNYTCRGSHP |
| | | HSPTGWSAPSNPMVIMVTGNHRKPSLLAHPGPLVKSGERVILQCWSDIMFE |
| | | HFFLHKEWISKDPSRLVGQIHDGVSKANFSIGSMMRALAGTYRCYGSVTHT |
| | | PYQLSAPSDPLDIVVTGLYEKPSLSAQPGPKVQAGESVTLSCSSRSSYDMYH |
| | | LSREGGAHERRLPAVRKVNRTFQADFPLGPATHGGTYRCFGSFRHSPYEWS |
| | | DPSDPLLVSVTGNPSSSWPSPTEPSSKSGNLRHLHILIGTSVVKIPFTILLFFLL |
| | | HRWCSNKKKCCCNGPRACREQK |
| 266 | TM/KIR2DS2 | SPTEPSSKTGNPRHLHVLIGTSVVKIPFTILLFFLLHRWCSNKKNAAVMDQEP |
| | domains | AGNRTVNSEDSDEQDHQEVSYA |
| 702 | KIR2DS2 Short | SPTEPSSKTGNPRHLHVLIGTSVVKIPFTILLFFLLHRWCSNKKNAAVMDQEP |
| | | AGNRTVNSEDSDEQDHQEVSYA |
| 703 | KIR2DS2 Short | TCACCCACTGAACCAAGCTCCAAAACCGGTAACCCCAGACACCTGCATG |
| | | TTCTGATTGGGACCTCAGTGGTCAAAATCCCTTTCACCATCCTCCTCTTCT |
| | | TTCTCCTTCATCGCTGGTGCTCCAACAAAAAAAATGCTGCTGTAATGGAC |
| | | CAAGAGCCTGCAGGGAACAGAACAGTGAACAGCGAGGATTCTGATGAA |
| | | CAAGACCATCAGGAGGTGTCATACGCA |
| 704 | Optimized | TCACCAACAGAGCCGTCAAGTAAAACCGGGAACCCGAGACATCTCCACG |
| | KIR2DS2 Short | TTCTGATCGGAACATCCGTCGTAAAAATCCCTTTCACCATATTGTTGTTTT |
| | | TCTTGTTGCACCGATGGTGCTCTAACAAGAAGAACGCAGCCGTAATGGA |
| | | CCAAGAGCCAGCCGGAAACAGGACTGTCAATTCCGAAGATTCCGATGAG |
| | | CAGGATCACCAAGAAGTTAGCTACGCA |
| 705 | TM/KIR2DS2 | PYEWSNSSDPLLVSVTGNPSNSWPSPTEPSSKTGNPRHLHVLIGTSVVKIPFTI |
| | Long | LLFFLLHRWCSNKKNAAVMDQEPAGNRTVNSEDSDEQDHQEVSYA |
| 706 | KIR2DS2 Long | CCCTATGAGTGGTCAAACTCGAGTGACCCACTGCTTGTTTCTGTCACAGG |
| | | AAACCCTTCAAATAGTTGGCCTTCACCCACTGAACCAAGCTCCAAAACC |
| | | GGTAACCCCAGACACCTGCATGTTCTGATTGGGACCTCAGTGGTCAAAAT |
| | | CCCTTTCACCATCCTCCTCTTCTTTCTCCTTCATCGCTGGTGCTCCAACAA |
| | | AAAAAATGCTGCTGTAATGGACCAAGAGCCTGCAGGGAACAGAACAGT |
| | | GAACAGCGAGGATTCTGATGAACAAGACCATCAGGAGGTGTCATACGCA |
| 707 | Optimized | CCCTATGAGTGGTCAAACTCGAGTGACCCACTGCTTGTTTCTGTCACAGG |
| | KIR2DS2 Long | AAACCCTTCAAATAGTTGGCCTTCACCAACAGAGCCGTCAAGTAAAACC |
| | | GGGAACCCGAGACATCTCCACGTTCTGATCGGAACATCCGTCGTAAAAAT |
| | | CCCTTTCACCATATTGTTGTTTTTCTTGTTGCACCGATGGTGCTCTAACAA |
| | | GAAGAACGCAGCCGTAATGGACCAAGAGCCAGCCGGAAACAGGACTGT |
| | | CAATTCCGAAGATTCCGATGAGCAGGATCACCAAGAAGTTAGCTACGCA |
| 753 | KIRSDS2 | VVKIP |
| | Association | |
| | Sequence | |

In some embodiments, the one or both of the first and second nucleic acid sequences each comprise a nucleic acid sequence encoding a TREM2 polypeptide in place of a KIR2DS2 domain. In some embodiments, the TREM2 polypeptide comprises one or more of a TREM2 signal sequences, extracellular domain, endogenous binder region, stalk region, transmembrane domain, and cytoplasmic domain of TREM2. In some embodiments, the TREM2 signal sequences, extracellular domain, endogenous binder region, stalk region, transmembrane domain, and cytoplasmic domain of TREM2 comprises the sequence of SEQ ID NO: 761, 762, 763, 764, 765, and 766, respectively. In some embodiments, the TREM2 signal sequences, extracellular domain, endogenous binder region, stalk region, transmembrane domain, and cytoplasmic domain of TREM2 comprises a functional variant of SEQ ID NO: 761, 762, 763, 764, 765, and 766, respectively, having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 761, 762, 763, 764, 765, and 766, respectively. In some embodiments, extracellular amino acid sequences encoded by one or both of the first and second nucleic acid sequences comprises at least a portion of an extracellular portion of a TREM2 polypeptide or a functional fragment thereof. In some embodiments, the transmembrane domain of one or both of the first and second nucleic acid sequences comprises at least a portion of a transmembrane domain of a TREM2 polypeptide or a functional variant thereof. In some embodiments, the portion of a transmembrane domain of a TREM2 polypeptide comprises the amino acid sequence of SEQ ID NO: 767, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 767. In some embodiments, one or both of the first nucleic acid sequence or the second nucleic acid sequence encode an intracellular domain. In some embodiments, the intracellular domain comprises an intracellular domain of a TREM2 polypeptide or a functional variant thereof. See Table 4. In some embodiments, the TREM2 polypeptide comprises the amino acid sequence of SEQ ID NO: 760. In some embodiments, the TREM2 polypeptide comprises a functional variant of the amino acid sequence of SEQ ID NO: 760 having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 760 See Table 4.2. In some embodiments, transmembrane domain of one or both of the first and second nucleic acid sequences comprises the TREM2 transmembrane domain of SEQ ID NO: 765 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 765.

In some embodiments, the TREM2 transmembrane domain encoded by the first nucleic acid sequence is configured to associate with DAP12 if the antigen-binding domain is associated with CD19. In some embodiments, the transmembrane domain encoded by the second nucleic acid sequence is configured to associate with DAP12 if the antigen-binding domain is associated with CD20.

TABLE 4
TREM2 Polypeptides

| SEQ | | |
|---|---|---|
| ID | | |
| NO: | Name: | Sequence: |
| 760 | TREM2 | MEPLRLLILLFVTELSGAHNTTVFQGVAGQSLQVSCPYDSMKHWGRRKAW |
| | | CRQLGEKGPCQRVVSTHNLWLLSFLRRWNGSTAITDDTLGGTLTITLRNLQ |
| | | PHDAGLYQCQSLHGSEADTLRKVLVEVLADPLDHRDAGDLWFPGESESFE |
| | | DAHVEHSISRSLLEGEIPFPPTSILLLLACIFLIKILAASALWAAAWHGQKPGT |
| | | HPPSELDCGHDPGYQLQTLPGLRDT |
| 761 | TREM2 Signal | MEPLRLLILLFVTELSGA |
| | Sequence | |
| 762 | TREM2 | HNTTVFQGVAGQSLQVSCPYDSMKHWGRRKAWCRQLGEKGPCQRVVSTH |
| | Extracellular | NLWLLSFLRRWNGSTAITDDTLGGTLTITLRNLQPHDAGLYQCQSLHGSEA |
| | Domain | DTLRKVLVEVLADPLDHRDAGDLWFPGESESFEDAHVEHSISRSLLEGEIPF |
| | | PPTS |
| 763 | TREM2 | GQSLQVSCPYDSMKHWGRRKAWCRQLGEKGPCQRVVSTHNLWLLSFLRR |
| | Endogenous | WNGSTAITDDTLGGTLTITLRNLQPHDAGLYQCQS |
| | Binder Region | |
| 764 | TREM2 Stalk | ADPLDHRDAGDLWFPGESESFEDAHVEHSISRSLLEGEIPFPPTS |
| | Region | |
| 765 | TREM2 | ILLLLACIFLIKILAASALWA |
| | Transmembrane | |
| | Domain | |
| 766 | TREM2 | AAWHGQKPGTHPPSELDCGHDPGYQLQTLPGLRDT |
| | Cytoplasmic | |
| | Domain | |
| 767 | TREM2 | LIKIL |
| | Association | |
| | Sequence | |

In some embodiments, each of the transmembrane and cytoplasmic regions of the first and second fusion proteins comprise a TREM2 polypeptide. In some embodiments, one or both of the first and second fusion proteins comprise a TREM2 polypeptide comprising a binder region, a stalk region, a transmembrane domain, and a cytoplasmic domain. In some embodiments, the binder region, the stalk region, the transmembrane domain, and the cytoplasmic domain comprise the amino acid sequence of SEQ ID NO: 763, 764, 765, and 766, respectively, or a function variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 763, 764, 765, and 766, respectively.

In some embodiments, the one or both of the first and second nucleic acid sequences each comprise a nucleic acid sequence encoding a TREM1 polypeptide in place of a KIR2DS2 domain. In some embodiments, the TREM1 polypeptide comprises one or more of a TREM1 stalk, a TREM1 transmembrane domain, and a TREM1 cytoplasmic domain. In some embodiments, the TREM1 stalk, the TREM1 transmembrane domain, and the TREM1 cytoplasmic domain comprise the amino acid sequences of SEQ ID NO: 768, 770, and 771, respectively, or a functional variant thereof comprising an amino acid sequence having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 768, 770, and 771, respectively. In some embodiments, the TREM1 stalk, the TREM1 transmembrane domain, and the TREM1 cytoplasmic domain comprise the amino acid sequences of SEQ ID NO: 768, 769, and 771, respectively, or a functional variant thereof comprising an amino acid sequence having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 768, 769, and 771, respectively. See Table 5.

TABLE 5
TREM1 Polypeptides

| SEQ | | |
|---|---|---|
| ID | | |
| NO: | Name: | Sequence: |
| 768 | TREM1 Full | KGFSGTPGSNENSTQNVYKIPP |
| | Length Stalk | TTTKALCPLYTSPRTVTQAPPK |
| | | STADVSTPDSEINLTNVTDIIR |
| | | VPVFN |
| 769 | TREM1 Short | TPDSEINLTNVTDIIRVPVFN |
| | Stalk | |
| 770 | TREM1 | IVILLAGGFLSKSLVFSVLFA |
| | Transmembrane | |
| | Domain | |
| 771 | TREM1 | VTLRSFVP |
| | Cytoplasmic | |
| | Domain | |
| 772 | TREM1 | LSKSL |
| | Association | |
| | Sequence | |

In some embodiments, the transmembrane domain encoded by the first nucleic acid sequence is configured to associate with DAP12 if the antigen-binding domain is associated with CD19. In some embodiments, the transmembrane domain encoded by the second nucleic acid sequence is configured to associate with DAP12 if the antigen-binding domain is associated with CD20.

As used herein, a “DAP12 association sequence” or a sequence that is “configured to associate with DAP12” can refer to an amino acid motif within a transmembrane domain that enables a fusion protein—such as a KIR-based CAR—to physically associate with the signaling adaptor DAP12 through electrostatic interaction. In the disclosed constructs, this association is mediated by the presence of a basic amino acid residue (typically arginine or lysine) positioned within the transmembrane helix, which pairs with the acidic transmembrane residue of DAP12.

In some embodiments, a fusion protein herein comprises a DAP12 amino acid sequence comprising SEQ ID NO: 667 or a functional variant thereof preceding a cleavage/ribosome skipping site. In some embodiments, the cleavage site is P2A (ATNFSLLKQAGDVEENPGP, SEQ ID NO: 677), E2A (QCTNYALLKLAGDVESNPGP, SEQ ID NO: 678), T2A (EGRGSLLTCGDVEENPGP, SEQ ID NO: 679), F2A (VKQTLNFDLLKLAGDVESNPGP, SEQ ID NO: 680), or furin (PRRAR, SEQ ID NO: 808), or functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 677-680 and 808. In some embodiments of a fusion protein herein, about 1, 2, 3, 4, 5, 6, 7, 8, 9, or 10 amino acids separate DAP12 or a functional variant thereof and a cleavage/ribosome skipping site. In some embodiments, the amino acids separating DAP12 or a functional variant thereof and a cleavage/ribosome skipping site comprise VEGGG (SEQ ID NO: 745). In some embodiments of a fusion protein herein, a DAP12 or a functional variant thereof precedes an anti-CD19 binder or an anti-CD20 binder. In some embodiments, a nucleic acid sequence encoding a fusion protein herein comprises an IRES site after a sequence encoding DAP12 or a functional variant and before a sequence encoding an anti CD19 or CD20 binder. In some embodiments of a fusion protein herein, a DAP12 or a functional variant thereof and a cleavage site precedes an anti-CD19 binder or an anti-CD20 binder. In some embodiments of a fusion protein herein, an arginine is inserted before one or both of an anti-CD19 binder or an anti-CD20 binder.

In some embodiments, a DAP12 polypeptide is replaced by DAP10 and a KIR polypeptide is replaced by a NKG2D polypeptide. In some embodiments, the third nucleic acid sequence encodes a DAP10 polypeptide in place of a DAP12 polypeptide. In some embodiments, the DAP10 polypeptide comprises an amino acid sequence of SEQ ID NO: 786, 787, 788, or 789, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 786, 787, 788, or 789. See Table 6. In some embodiments, DAP12 is replaced by an amino acid sequence comprising SEQ ID NO: 786, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 786. In some embodiments, a DAP12 transmembrane domain is replaced by an amino acid sequence comprising SEQ ID NO: 787, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 787. In some embodiments, a DAP12 cytoplasmic domain is replaced by an amino acid sequence comprising SEQ ID NO: 788, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 788. In some embodiments, a DAP12 interaction sequences is replaced by an amino acid sequence comprising SEQ ID NO: 789, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 789. In some embodiments, one or both of the first nucleic acid sequence or the second nucleic acid sequence encode a NKG2D polypeptide in place of a KIR2DS2 polypeptide of any KIR2DS2 polypeptide containing fusion protein herein. In some embodiments, the NKG2D polypeptide comprises the amino acid sequence of SEQ ID NO: 790, 791, 792, 793, 794, or 795, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 790, 791, 792, 793, 794, or 795. In some embodiments, a KIR2DS2 polypeptide of any fusion protein herein is replaced with an amino acid sequence comprising the sequence of SEQ ID NO: 790, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 790. In some embodiments, an extracellular domain of a KIR2DS2 polypeptide of any fusion protein herein is replaced by one or both of SEQ ID NO: 791 and 792, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to one or both of SEQ ID NOS: 791 and 792. In some embodiments, the transmembrane domain of a KIR2DS2 polypeptide of any fusion protein herein is replaced by an amino acid sequence comprising the sequence of SEQ ID NO: 793, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to one or both of SEQ ID NO: 793. In some embodiments, the cytoplasmic domain of a KIR2DS2 polypeptide of any fusion protein herein is replaced by an amino acid sequence comprising the sequence of SEQ ID NO: 794, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to one or both of SEQ ID NO: 794. In some embodiments, the association sequence of a KIR2DS2 polypeptide of any fusion protein herein is replaced by an amino acid sequence comprising the sequence of SEQ ID NO: 795, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to one or both of SEQ ID NO: 796.

TABLE 6
DAP10 and NKG2D Sequences

| SEQ | | |
|---|---|---|
| ID | | |
| NO: | Name: | Sequence: |
| 786 | DAP10 | MIHLGHILFLLLLPVAAAQTTP |
| | | GERSSLPAFYPGTSGSCSGCGS |
| | | LSLPLLAGLVAADAVASLLIVG |
| | | AVFLCARPRRSPAQEDGKVYIN |
| | | MPGRG |
| 787 | DAP 10 | LLAGLVAADAVASLLIVGAVF |
| | Trans- | |
| | membrane | |
| | Domain | |
| 788 | DAP10 | LCARPRRSPAQEDGKVYINMPG |
| | Cytoplasmic | RG |
| | Domain | |
| 789 | DAP10 | AADAV |
| | Interaction | |
| | Sequence | |
| 790 | NKG2D | MGWIRGRRSRHSWEMSEFHNYN |
| | | LDLKKSDFSTRWQKQRCPVVKS |
| | | KCRENASPFFFCCFIAVAMGIR |
| | | FIIMVTIWSAVFLNSLFNQEVQ |
| | | IPLTESYCGPCPKNWICYKNNC |
| | | YQFFDESKNWYESQASCMSQNA |
| | | SLLKVYSKEDQDLLKLVKSYHW |
| | | MGLVHIPTNGSWQWEDGSILSP |
| | | NLLTIIEMQKGDCALYASSFKG |
| | | YIENCSTPNTYICMQRTV |
| 791 | NKG2D | PCPKNWICYKNNCYQFFDESKN |
| | Binder | WYESQASCMSQNASLLKVYSKE |
| | Region | DQDLLKLVKSYHWMGLVHIPTN |
| | | GSWQWEDGSILSPNLLTIIEMQ |
| | | KGDCALYASSFKGYIENCSTPN |
| | | TYICMQ |
| 792 | NKG2D Stem | IWSAVFLNSLFNQEVQIPLTES |
| | | YCG |
| 793 | NKG2D | PFFFCCFIAVAMGIRFIIMVT |
| | Trans- | |
| | membrane | |
| | Domain | |
| 794 | NKG2D | MGWIRGRRSRHSWEMSEFHNYN |
| | Cytoplasmic | LDLKKSDFSTRWQKQRCPVVKS |
| | Domain | KCRENAS |
| 795 | NKG2D | GIRFI |
| | Association | |
| | Sequence | |

### Exemplary Anti-CD19 and Anti-CD20 KIR-CARs

In some embodiments, the transmembrane domain of the anti-CD19 and anti-CD20 KIR-CAR comprises a KIR polypeptide comprising the amino acid sequence VLIGTSVVKIPFTILLFFLLH (SEQ ID NO: 742) or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742.

In some embodiments, the first fusion protein and/or second fusion protein comprises an extracellular portion comprising a KIR polypeptide (e.g., KIR stem domain) after the anti-CD19 or anti-CD20 antigen binding domain. In some embodiments, the extracellular portion (e.g., KIR stem domain) is a short stem domain. In some embodiments, the extracellular portion (e.g., KIR stem domain) is a long stem domain. In some embodiments, the extracellular portion (e.g., KIR stem domain) comprises a KIR polypeptide comprising SPTEPSSKTGNPRHLH (SEQ ID NO: 740) or PYEWSNSSDPLLVSVTGNPSNSWPSPTEPSSKTGNPRHLH (SEQ ID NO: 741) or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 740 or 741.

In some embodiments, the first fusion protein and second fusion protein each comprises an intracellular domain comprising a KIR polypeptide comprising RWCSNKKNAAVMDQEPAGNRTVNSEDSDEQDHQEVSYA (SEQ ID NO: 743) or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743.

In some embodiments, the first fusion protein comprises a KIR extracellular portion, transmembrane domain, and intracellular domain that together comprise the amino acid sequence SPTEPSSKTGNPRHLHVLIGTSVVKIPFTILLFFLLHRWCSNKKNAAVMDQEPAGNRTV NSEDSDEQDHQEVSYA ((SEQ ID NO: 266), Table 1D). In some embodiments, the first fusion protein comprises a KIR extracellular portion, transmembrane domain, and intracellular domain that together comprise the amino acid sequence of a functional variant of the amino acid sequence of SEQ ID NO: 266 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 266. In some embodiments, the second fusion protein comprises a KIR extracellular portion, transmembrane domain, and intracellular domain that together comprise the amino acid sequence comprise the amino acid sequence PYEWSNSSDPLLVSVTGNPSNSWPSPTEPSSKTGNPRHLHVLIGTSVVKIPFTILLFFLLH RWCSNKKNAAVMDQEPAGNRTVNSEDSDEQDHQEVSYA ((SEQ ID NO: 744), Table 1D). In some embodiments, the second fusion protein comprises a KIR extracellular portion, transmembrane domain, and intracellular domain that together comprise the amino acid sequence comprise the amino acid sequence of a functional variant of the amino acid sequence of SEQ ID NO: 744 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 744.

In various embodiments, the fusion proteins disclosed herein comprise one or more flexible glycine-serine (GS) linkers positioned between an antigen-binding domain and a KIR-derived extracellular/stem region to facilitate optimal orientation, mobility, and spatial separation of these domains. The GS linker can include any suitable arrangement of glycine and serine residues, including, for example, (G₄S), (G₄S)₂, (G₄S)₃, (G₄S)₅, (G₃S), (G₂S), (GS)ₙ, or combinations thereof. In some embodiments, the GS linker is operably disposed between an anti-CD19 antigen-binding domain and a KIR stem region; in other embodiments, the GS linker is positioned between an anti-CD20 antigen-binding domain and a KIR stem region. Additional embodiments include constructs in which both anti-CD19 and anti-CD20 domains are independently linked to their respective KIR stems by GS-based linkers, allowing selective tuning of each CAR arm to accommodate differences in antigen accessibility, membrane proximity, or epitope orientation. In some embodiments, the GS linker may be replaced with or supplemented by alternative flexible linkers, such as GSGGG, SGGGS, (SGGGS)₂, (GGGGS)ₙ, or hybrid linkers incorporating alanine, threonine, or proline residues to adjust rigidity or length. Without being bound by theory, these GS-based linkers and their variants enhance antigen engagement by permitting the antigen-binding domain to adopt favorable conformations while maintaining productive association with the underlying KIR2D stem and DAP12 signaling machinery. Accordingly, GS linkers and functional variants thereof provide a modular design element suitable for CAR constructs targeting CD19, CD20, or additional antigens in mono-, dual-, or multi-specific formats.

In some embodiments, the first fusion protein comprises anti-CD19 antigen-binding domain linked to a short KIR stem region by a (G₄S)₂ linker. As used in the Examples herein, the term “KIRShort” when used in connection with a binder as described herein refers to a short KIR2DS2 stem linked to the binder by a (G₄S)₂ linker.

In some embodiments, the second fusion protein comprises anti-CD20 antigen-binding domain linked to a long KIR stem region by a (G₄S)₅ linker. As used in the Examples herein, the term “KIRLong” when used in connection with a binder as described herein refers to a long KIR2DS2 stem linked to the binder by a (G₄S)₅ linker.

In some embodiments, a first short amino acid sequence interposes between the leader sequence and the antigen binding domain, such as the CD19 targeting domain and/or the CD20 targeting domain. In some embodiments, a second short amino acid sequence interposes between the anti-CD19 antigen binding domain and the tag. In some embodiments, the first short amino acid sequence is 1, 2, 3, or 4 amino acids in length. In some embodiments, the first short amino acid sequence is 2 amino acids in length. In some embodiments, the first short amino acid sequence is GS. In some embodiments, the second short amino acid sequence is 1, 2, 3, or 4 amino acids in length. In some embodiments, the second short amino acid sequence is 2 amino acids in length. In some embodiments, the second short amino acid sequence is AS.

In some embodiments, the first fusion protein comprises the amino acid sequence of one of SEQ ID NOS: 370-412. In some embodiments, the first fusion protein comprises the amino acid sequence of a functional variant of the amino acid sequence of one of SEQ ID NOS: 370-412 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of the respective one of SEQ ID NOS: 370-412.

TABLE 7
Exemplary CAR Construct Features and Exemplary Anti-CD19 KIR-CARs

| SEQ | | |
|---|---|---|
| ID | | |
| NO: | Name: | Sequence: |
| 262 | scFv KIR | ATGGCCTTACCAGTGACCGCCTTGCTCCTGCCGCTGGCCTTGCTGCTCCACGC |
| | construct | CGCCAGGCCGGGATCCXGCTAGCGAGCAGAAATTAATCTCAGAGGAGGACCT |
| | DNA | GCCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCTCA |
| | | GTGGTCAAAATCCCTTTCACCATCCTCCTCTTCTTTCTCCTTCATCGCTGGTGC |
| | | TCCAACAAAAAAAATGCTGCTGTAATGGACCAAGAGCCTGCAGGGAACAGA |
| | | ACAGTGAACAGCGAGGATTCTGATGAACAAGACCATCAGGAGGTGTCATACG |
| | | CATAA |
| | | (X indicates scFv insertion site) |
| 263 | scFv KIR | MALPVTALLLPLALLLHAARPGSXASEQKLISEEDLPSGGGGSGGGGSSPTEPSS |
| | construct | KTGNPRHLHVLIGTSVVKIPFTILLFFLLHRWCSNKKNAAVMDQEPAGNRTVNS |
| | AA | EDSDEQDHQEVSYA |
| | | (X indicates scFv insertion site) |
| 264 | Myc tag | GAGCAGAAATTAATCTCAGAGGAGGACCTG |
| 746 | Myc tag | EQKLISEEDL |
| 755 | 6X His tag | HHHHHH |
| 756 | Flag tag | DYKDDDDK |
| 757 | Strep Tag II | WSHPQFEK |
| 758 | SBP | MDEKTTGWRGGHVVEGLAGELEQLRARLEHHPQGQREP |
| | (Streptavidin | |
| | Binding | |
| | Protein | |
| 759 | HA | YPYDVPDYA |
| | (hemaggltinin) | |
| 260 | Linker | SGGGGSGGGGS |
| 811 | Linker | SGGGGSGGGGSGGGGSGGGGSGGGGS |
| 821 | (G4S)n | GGTGGCGGAGGTTCT |
| | Linker | |
| | DNA 1 | |
| 822 | (G4S)n | GGAGGTGGGGGTTCC |
| | Linker | |
| | DNA 2 | |
| 823 | (G4S)n | GGAGGCGGCGGCAGC |
| | Linker | |
| | DNA 3 | |
| 824 | (G4S)n | GGCGGGGGAGGGTCC |
| | Linker | |
| | DNA 4 | |
| 825 | (G4S)n | GGAGGGGGTGGTTCT |
| | Linker | |
| | DNA 5 | |
| 826 | (G4S)n | GGTGGAGGAGGATCG |
| | Linker | |
| | DNA 6 | |
| 827 | (G4S)n | GGAGGCGGTGGCAGC |
| | Linker | |
| | DNA 7 | |
| 828 | (GSGGS)n | GGCAGCGGCGGCAGC |
| | Linker | |
| 829 | (GGGS)n | GGCGGTGGTTCC |
| | Linker | |
| 830 | Linker A | GGGSSRSSSSGGGGSGGGG |
| 831 | Linker A | GGCGGTGGTTCCTCTAGATCTTCCTCCTCTGGTGGCGGTGGCTCGGGCGGTG |
| | DNA | GTGGG |
| 832 | Whitlow | GSTSGSGKPGSGEGSTKG |
| | Linker | |
| 833 | Whitlow | GGCAGCACATCCGGTTCCGGGAAGCCCGGCTCCGGAGAGGGCAGCACCAAG |
| | Linker | GGG |
| | DNA | |
| 265 | TM/KIR2D | CCCACTGAACCAAGCTCCAAAACCGGTAACCCCAGACACCTGCATGTTCTGA |
| | S2 domains | TTGGGACCTCAGTGGTCAAAATCCCTTTCACCATCCTCCTCTTCTTTCTCCTTC |
| | | ATCGCTGGTGCTCCAACAAAAAAAATGCTGCTGTAATGGACCAAGAGCCTGC |
| | | AGGGAACAGAACAGTGAACAGCGAGGATTCTGATGAACAAGACCATCAGGA |
| | | GGTGTCATACGCA |
| 266 | TM/KIR2D | SPTEPSSKTGNPRHLHVLIGTSVVKIPFTILLFFLLHRWCSNKKNAAVMDQEPAG |
| | S2 short | NRTVNSEDSDEQDHQEVSYA |
| | stem | |
| | domains | |
| 740 | KIR Short | SPTEPSSKTGNPRHLH |
| | Extracellular | |
| | portion | |
| | (stem) | |
| 741 | KIR Long | PYEWSNSSDPLLVSVTGNPSNSWPSPTEPSSKTGNPRHLH |
| | Extracellular | |
| | portion | |
| | (stem) | |
| 742 | KIR | VLIGTSVVKIPFTILLFFLLH |
| | Transmembrane | |
| | domain | |
| 743 | KIR | RWCSNKKNAAVMDQEPAGNRTVNSEDSDEQDHQEVSYA |
| | Intracellular | |
| | Domain | |
| 744 | KIR Long | PYEWSNSSDPLLVSVTGNPSNSWPSPTEPSSKTGNPRHLHVLIGTSVVKIPFTILL |
| | (TM plus | FFLLHRWCSNKKNAAVMDQEPAGNRTVNSEDSDEQDHQEVSYA |
| | long stem) | |
| 357 | CD8 | MALPVTALLLPLALLLHAARP |
| | Leader | |
| | Sequence | |
| 698 | CD8 | ATGGCCTTACCAGTGACCGCCTTGCTCCTGCCGCTGGCCTTGCTGCTCCACGC |
| | Leader | CGCCAGGCCG |
| | Sequence | |
| 699 | Optimized | ATGGCACTCCCCGTCACCGCGCTCCTCTTGCCCCTCGCGCTGCTCCTTCATGC |
| | CD8 | TGCGAGGCCG |
| | Leader | |
| | Sequence | |
| 837 | GMCSFR | MLLLVTSLLLCELPHPAFLLIP |
| | Leader | |
| 700 | GMCSFR | ATGCTTCTCCTGGTGACAAGCCTTCTGCTCTGTGAGTTACCACACCCAGCATT |
| | Leader | CCTCCTGATCCCA |
| 701 | Optimized | ATGCTTCTGTTGGTGACTTCACTCCTGCTGTGCGAACTTCCTCATCCCGCCTT |
| | GMCSFR | CCTTTTGATTCCA |
| | Leader | |
| 674 | GM-CSF | MWLQNLLLLGAVVCSIS |
| | Leader | |
| | Sequence | |
| 675 | IgK Leader | METPAQLLFLLLLWLPDTTG |
| | Sequence | |
| 676 | IL-22 | MAALQKSVSSFLMGTLATSCLLLLALLVQGGAA |
| | Leader | |
| | Sequence | |
| 754 | IgK Leader | METPAQLLFLLLLWLPDTTG |
| 370 | scFv_01 | MALPVTALLLPLALLLHAARPGSQAVLNQPASVSAALGQRVTISCNTNIGSPYDV |
| | CAR | QWYQQLPGKSPKTIIYGNSNRPSGVPVRFSGSKSGSTATLTIAGIQAEDEADYYC |
| | | QSYDDSLDGRVVFGGGTQLTVLGGGSSRSSSSGGGGSGGGGEVQLVESGGDLV |
| | | KPGGSLRLSCVASGFTFSNHEMYWVRQAPGKGLEWVARIYDSGSRTRYADAVK |
| | | GRFTISRDNAKNTLYLQMNSLTAEDTAVYFCAGGNSRGWTEFGMDYWGPGTLV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 371 | scFv_02 | MALPVTALLLPLALLLHAARPGSSYVLTQSPSLSGSLGQKVTISCTGSRSNLGGN |
| | CAR | YVGWYQQVPGMAPRNVIYANDFRPSRIPARFSASKSGTSATLTISGLQAEDEAD |
| | | YFCSSWDDDLGSHVFGSGTQLTILGGGSSRSSSSGGGGSGGGGEEQLVEFGGDL |
| | | VKPGGSLRLSCVASGFSFSSYDMSWVRQAPGKGLQWVASTNFDGSRTYYTDAV |
| | | EGRFTISRDNARNTVYLQMNSLRAEDTAVYYCARGMGSWGASTFGYWGQGTL |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 372 | scFv_03 | MALPVTALLLPLALLLHAARPGSQSVLNQPPSVSGALGQTVTISCSGSHTNIGSG |
| | CAR | FDVQWYQQLPGKSPQTLVYANINRPSGVPARFSGSKSGSTATLTITGVQAEDEAD |
| | | YYCQSYDDNFDGHVFGSGTQLTVLGGGSSRSSSSGGGGSGGGGEVQLVQSGGD |
| | | LVKPGGSLRLSCVASGFTFSNYTMSWVRQAPGQGLQWVAYINYDGSITYYAEAV |
| | | KGRFTVSRDNAKNTLYLQMNSLRAEDTAVYFCVKETFMIPTEWGQGTLITVSSA |
| | | SEQKLISEEDLPSGGGGSGGGGSSPTEPSSKTGNPRHLHVLIGTSVVKIPFTILLFF |
| | | LLHRWCSNKKNAAVMDQEPAGNRTVNSEDSDEQDHQEVSYA |
| 373 | scFv_04 | MALPVTALLLPLALLLHAARPGSQSVLNQPPSVSGALGQTVTISCSGSHTNIGSG |
| | CAR | FDVQWYQQLPGKSPQTIIYANINRPSGVPARFSGSKSGSTATLTITGVQAEDEAD |
| | | YYCQSYDDNFDYVFGSGTQLTVLGGGSSRSSSSGGGGSGGGGEEQLVEFGGDL |
| | | VKPGGSLRLSCVVSGFDFSRYGMSWVRQSPGKGLQWVADISYRGTTFYADDVK |
| | | GRFTVSRDSAKNTLYLEMTSLTVEDTAVYYCANSANWYVSPFDHWGQGTLVTV |
| | | SSASEQKLISEEDLPSGGGGSGGGGSSPTEPSSKTGNPRHLHVLIGTSVVKIPFTIL |
| | | LFFLLHRWCSNKKNAAVMDQEPAGNRTVNSEDSDEQDHQEVSYA |
| 374 | scFv_05 | MALPVTALLLPLALLLHAARPGSQPVLTQPPSVSGSLGQRVTISCSGSTNTIGVV |
| | CAR | GANWYQQLPGKSPKLLVYSDGVRPSGVPDRFSGSRSANSDTLTITGLQAEDEAD |
| | | YYCQSFDSTHNAIVFGGGTHLTVLGGGSSRSSSSGGGGSGGGGEVQLVETGGDL |
| | | VKPAGSLRLSCAASGFPFSGYSMTWVRQAPEKGLQLVAGINSDGSHTYYTDDV |
| | | KGRFTISRDNTKYILYLQMNSLRAEDTAMYYCGSHSWGNFHYWGQGTLVTVSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 375 | scFv_06 | MALPVTALLLPLALLLHAARPGSQPVLTQPPSVSGSLGQRVTISCTGTNTNIGSG |
| | CAR | YSVQWYQQLPGESPKPIIYGSSNRPSGVPARFSGSKSGSTGTLTITGIQAEDEADY |
| | | HCQSYDDSLDGHAVFGGGTQLTILGGGSSRSSSSGGGGSGGGGEVQLVQSGGDL |
| | | VKPGGSLRLSCVASGFTFSDNYMSWIRQVPGKGLQWVSRISTDGRGTSYADAV |
| | | KGRFTISRDNAKNTLYLQMNSLRDEDTAVYYCARRSGIWGFDYWGQGALVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 376 | scFv_07 | MALPVTALLLPLALLLHAARPGSSYELTQPPSMSAALGQRVTISCTGSNTNIGSG |
| | CAR | YDVQWYQQVPGKSPTPLIYGNTNRPSGVPVRFSGSKSGNTATLTITGIQAEDEAD |
| | | YYCQSYDDNFDGHHVFGSGTQLTVLGGGSSRSSSSGGGGSGGGGEVQLVESGG |
| | | DLVKPAGSLRLSCVASGFTFSNYGMNWVRQAPGKGLQWVAGINSGGSTTTYAD |
| | | VVKGRFTISRDNAKNTLYLQMNSLGAEDTAVYYCARVDYSSSWLVDYWGQGT |
| | | LVTVSSASEQKLISEEDLPSGGGGSGGGGSSPTEPSSKTGNPRHLHVLIGTSVVKI |
| | | PFTILLFFLLHRWCSNKKNAAVMDQEPAGNRTVNSEDSDEQDHQEVSYA |
| 377 | scFv_08 | MALPVTALLLPLALLLHAARPGSQSVLTQPASVSGSLGQKVTISCTGSRSNIGAN |
| | CAR | YVGWYQQVPGIGPRTVIFGYNYRPSGVPDRFSSSKSGNSATLTISGLQAEDEAEY |
| | | YCSSWDDSLRGTVFGSGTQLTVLGGGSSRSSSSGDGGSGGGGEVQLVESGGDLV |
| | | KPGESLRLSCVASGFTFSSYGMSWVRQSPGKGLQWVADISYGGGTYYADAVKG |
| | | RFTISRDNARNTVYLQMNSLRAEDTAVYYCASQWGDWGHFEYWGQGTLVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 378 | scFv_09 | MALPVTALLLPLALLLHAARPGSQPVLTQPPSVSAALGQRVTLSCTGSKTNIGSG |
| | CAR | YDVQWYQQFPGKSPKTIIYGNSNRFSGVPARFSGSKSGSIATLTITGIQAEDEADY |
| | | YCQSFDDNVDGYVFGSGTQLTILGGGSSRSSSSGGGGSGGGGEVQLVESGGDLV |
| | | KPGGSLRLSCVASGFTFSSDSIHWVRQAPGKGLQWVAYINSGGSTTYYADAVKG |
| | | RFTISRDNARNTVYLKMNSLRAEDTAVYYCAKDSGFGSWYLESWGQGTLVTVS |
| | | PASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 379 | scFv_10 | MALPVTALLLPLALLLHAARPGSQAVLNQPASVSGALGQTVTISCSGSHTNIGSG |
| | CAR | FDVQWYQQLPGKSPQTIIYANINRPSGVPARFSGSKSGSTATLTITGVQAEDEAD |
| | | YHCQSFDDDLDGPVFGGGTHLTVLGGGSSRSSSSGGGGSGGGGEVQLVQSGGD |
| | | LVKPGGSLKISCVASGLTFNRNYMTWIRQAPGKGLQWVSEINPDGSTTSYTDAV |
| | | RGRFTISRDNAKNTLYLQMNSMRDEDTAVYYCARSSWGSFDYWGQGTLVTVSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 380 | scFv_11 | MALPVTALLLPLALLLHAARPGSQPVLTQPPSVSGALGQTVTISCSGSHTNIGSG |
| | CAR | FDVQWYQQLPGKSPQTLVYANINRPSGVPARFSGSKSGSTATLTITGVQAEDEAD |
| | | YYCQSYDDNFDYVFGSGTQLTVLGGGSSRSSSSGGGGSGGGGEVQLVESGGDL |
| | | VKPGGSLRLSCVASGFTFSDYSMSWVRQSPGKGLQWVAAIMTSGSTYYADAVK |
| | | GRFTISRDNAKNTLYLQMDSLRAEDTAVYYCAHDYWFDFWGQGTLVTVSSASE |
| | | QKLISEEDLPSGGGGSGGGGSSPTEPSSKTGNPRHLHVLIGTSVVKIPFTILLFFLL |
| | | HRWCSNKKNAAVMDQEPAGNRTVNSEDSDEQDHQEVSYA |
| 381 | scFv_12 | MALPVTALLLPLALLLHAARPGSSYELTQPPSATVTLRQTARLTCGGENIGRKSV |
| | CAR | QWYQQKPGQSPLLIIYADSSRPSGIPERFSGANSGNTASLTISGALAEDEADYYCQ |
| | | IWDRNINVFGSGTQLTVLGGGSSRSSSSGGGGSGGGGEEQLVEFGGDLVKPAGS |
| | | LRLSCVASGFTFSSYDMSWVRQAPGKGLQWVATISYDGSSTYYTDAVKGRFTVS |
| | | RDNAKNTLYLQMNSLRAEDTALYYCAKDSGSWSHFYGLDTWGHGTSVFVSSA |
| | | SEQKLISEEDLPSGGGGSGGGGSSPTEPSSKTGNPRHLHVLIGTSVVKIPFTILLFF |
| | | LLHRWCSNKKNAAVMDQEPAGNRTVNSEDSDEQDHQEVSYA |
| 382 | scFv_13 | MALPVTALLLPLALLLHAARPGSQPVLTQPPSVSGALGQTVTISCSGSHTNIGSG |
| | CAR | FDVQWYQQLPGKSPQTLVYANINRPSGVPARFSGSKSGSTATLTITGVQAEDEAD |
| | | YYCQSYDDNFDYVFGSGTQLTVLGGGSSRSSSSGDGGSGGGGEVQLVESGGGL |
| | | MKPGGSLKLSCLASGFTFSRYYMYWVRQAPGKGLQWVAQISSDGRRTYYADA |
| | | VQGRFTISRDNVKNTVYLQMNSLSDEDTAVYYCVREDYFDFDYWGQGTLVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 383 | scFv_14 | MALPVTALLLPLALLLHAARPGSQSVLNQPPSVSAALGQRITISCTGSHTNIGSG |
| | CAR | YDVQWYQQLPGKSPRTIIYGNSDRPSGVPVRFSGSKSGNTATLTITGIQAEDEAD |
| | | YYCQSYDDNLNDPYVFGSGTQLTVLGGGSSRSSSSGGGGSGGGGEVQLVQSGG |
| | | DLVKPGGSLRLSCVASGFAFSGKTMQWVRQAPGKGLQWVAYIQHDGSRTSYAD |
| | | AVKGRFTISRDNAKNTLYLQMDSLRAEDTAVYYCADHPWYGPEYWGQGTPVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 384 | scFv_15 | MALPVTALLLPLALLLHAARPGSSYELTQPPSVSAALGQGVTISCIGSDTNIGNH |
| | CAR | YDVQWYQQLPGKSPRTIIYGNSDRPSGVPGRFSGSKSGNTATLTITGIQAEDEAD |
| | | YHCQSYDDDLDGHIVFGGGTHLTVLGGGSSRSSSSGGGGSGGGGEVQLVQSGG |
| | | DLVKPGGSLRLSCVASGFTFNKYHMSWVRQAPGKGLQWVGKITSDGSATGYAD |
| | | AVKGRFTISRDNAKNTFYLQMDSLRAEDTAMYYCARDQSSWGTGNFDYWGQG |
| | | TLVTVSSASEQKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDHQEVSYA |
| 385 | scFv_16 | MALPVTALLLPLALLLHAARPGSQSVLTQPTSVSGSLGQRVTISCTGSSSNVGYG |
| | CAR | NYVGWYQQLPGTGPRTLIYGSHYRPSGVPDRFSGSSSGSSATLTISGLQAEDEAD |
| | | YYCSSYDSTLSGYVFGSGTQLTILGGGSSRSSSSGGGGSGGGGEVQLVESGGDLV |
| | | KPGGSLRLSCVGSGFTLSSYWMSWVRQAPGKGLQWIAEISGSGNTIYYGDAVK |
| | | GRFTISRDNAKNTLYLQMNSLRDEDTAVYYCATEYYWNIDNWGQGTLVTVSSA |
| | | SEQKLISEEDLPSGGGGSGGGGSSPTEPSSKTGNPRHLHVLIGTSVVKIPFTILLFF |
| | | LLHRWCSNKKNAAVMDQEPAGNRTVNSEDSDEQDHQEVSYA |
| 386 | scFv_17 | MALPVTALLLPLALLLHAARPGSQPVLTQPPSVSAALGQRVTISCTGSKTNIGSG |
| | CAR | YDVQWYQQLPGKSPKTIIYGNSNRPSGVPARFSGSKSGSIATLTITGIQAEDEADY |
| | | YCQSFDDNVDGYVFGSGTQLTILGGGSSRSSSSGGGGSGGGGEVPLVESGGDLV |
| | | KPGGSLRLSCVASGFTFSSYGMSWVRQAPGKGLQWVAYINGGGSSTSYADAVR |
| | | GRFTISRDNAENTLYLQMNSLRADDTAVYYCSNYYGIYWDSDYWGQGTLVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 387 | scFv_18 | MALPVTALLLPLALLLHAARPGSQSALTQTASVSGSLGQKVTISCTGSRSNIGAN |
| | CAR | YVGWYQQVPGIGPRTVIFGYNYRPSGVPDRFSSSKSGNSATLTISGLQAEDEAEY |
| | | YCSTWDSSLNAYVFGSGTQLTVLGGGSSRSSSSGGGGSGGGGEVPLVESGGDLV |
| | | KPGGSLRLSCVASGFTLSNYGMSWVRQSPGKGLQWVAAIRSGGNTYYADAVKG |
| | | RFTISRDNAKNTLYLQMNSLRAEDTAVYYCASQWGDWGHFEYWGQGTLVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 388 | scFv_19 | MALPVTALLLPLALLLHAARPGSQAVLTQPASVSGPLGQRVTISCSGSTNNIGIVG |
| | CAR | AAWYQQFPGKAPKLLVYSDGSRPSGVPDRFSGSNSGNSATLTISGLQAEDEADY |
| | | YCQSVDATHGTILFGGGTQLTVLGGGSSRSSSSGGGGSGGGGEVQLVETGGDLV |
| | | KPAGSLRLSCAASGFPFSGYSMTWVRQAPEKGLQLVAGINSDGSHTYYTDDVK |
| | | GRFTVSRDNTKYILYLQMNSLRVEDTAIYYCGSHSWGNFHYWGQGTLVTVSSA |
| | | SEQKLISEEDLPSGGGGSGGGGSSPTEPSSKTGNPRHLHVLIGTSVVKIPFTILLFF |
| | | LLHRWCSNKKNAAVMDQEPAGNRTVNSEDSDEQDHQEVSYA |
| 389 | scFv_20 | MALPVTALLLPLALLLHAARPGSQSVLNQPPSVSVSLGQTATISCSGESLSKYYA |
| | CAR | QWFQQKAGQVPVLVIYKDTERPSGIPDRFSGSSSGNTHTLTISGARAEDEADYY |
| | | CESEVSPDTIVFGGGTHLTVLGGGSSRSSSSGGGGSGGGGEVQLVESGGDLVKP |
| | | GGSLRLSCVASGFTFSSYAMSWVRQAPGEGLQWVAYINSAGSTTYYADAVKGR |
| | | FTISRDNARNTVYLQMNSLRAEDTAVYYCGRYYYGLDYWGPGTSVFVSSASEQ |
| | | KLISEEDLPSGGGGSGGGGSSPTEPSSKTGNPRHLHVLIGTSVVKIPFTILLFFLLH |
| | | RWCSNKKNAAVMDQEPAGNRTVNSEDSDEQDHQEVSYA |
| 390 | scFv_21 | MALPVTALLLPLALLLHAARPGSQPVLTQPPSVSVSLGQTATISCSGESLSKYYA |
| | CAR | QWFQQKAGQAPVLVIYKDTERPSGIPDRFSGSSSGNTHTLTISGARAEDEADYY |
| | | CESAVSSDTAVFGSGTQLTVLGGGSSRSSSSGGGGSGGGGEVQLVESGGDMVKP |
| | | GGSLRLSCVASGFTFSSFWMYWVRQAPGKGLQWVGGINSGGSITSYADAVKGR |
| | | FTISRDNARNTLYLQMDSLRDEDTAVYYCAKPDIYGSFDHWGQGTLVTVSSASE |
| | | QKLISEEDLPSGGGGSGGGGSSPTEPSSKTGNPRHLHVLIGTSVVKIPFTILLFFLL |
| | | HRWCSNKKNAAVMDQEPAGNRTVNSEDSDEQDHQEVSYA |
| 391 | scFv_22 | MALPVTALLLPLALLLHAARPGSQPVLTQPPSVSGSLGQRVTISCTGSSSNVGYG |
| | CAR | DYVGWYQQLPGTSPRTLIYSSSSRPSGVPDRFSGSRSGSTATLTISGLQAEDEADY |
| | | HCSSYDTSLNGFVFGSGTQLTILGGGSSRSSSSGGGGSGGGGELTLQESGPGLVK |
| | | PAQTLSLTCTVSGDSVTSSHFWHWIRQRPGRGLEWMGYWSDITIYSPTFQGRISI |
| | | TVDTARNQFSLHLNSVTTEDSAVYFCARYNGYVDYLDVWGQGTLITVSSASEQ |
| | | KLISEEDLPSGGGGSGGGGSSPTEPSSKTGNPRHLHVLIGTSVVKIPFTILLFFLLH |
| | | RWCSNKKNAAVMDQEPAGNRTVNSEDSDEQDHQEVSYA |
| 392 | scFv_23 | MALPVTALLLPLALLLHAARPGSDIVMTQTPLSLSVSPGEPASISCKASQSLLHSN |
| | CAR | GNTYLYWFRQKPGQSPQRLIYLVSNRDAGVPDRFSGSGSGTDFTLRISRVEADDS |
| | | GIYYCGQGKQDPITFGEGTHLEIKGGSSRSSSSGGGGSGGGGELTLQESGPGLVK |
| | | PSQTLSLTCVVSGGSVTTSDYWNWLRQRPGRGLEYMGYWKGRTEYNPAFQGRI |
| | | SITADTAKNQFSLQLNSVTADDTAIYYCAHWGSGNLDYWGPGTLVTVSSASEQK |
| | | LISEEDLPSGGGGSGGGGSSPTEPSSKTGNPRHLHVLIGTSVVKIPFTILLFFLLHR |
| | | WCSNKKNAAVMDQEPAGNRTVNSEDSDEQDHQEVSYA |
| 393 | scFv_24 | MALPVTALLLPLALLLHAARPGSQSVLNQPPSVSGALGQTVTISCSGSHTNIGSG |
| | CAR | FDVQWYQQLPGKSPQTLVYANINRPSGVPVRFSGSKSGTTATLTITGIQAEDEAD |
| | | YYCQSYDDNVDGYVFGSGTQLTVLGGGSSRSSSSGGGGSGGGGEVQLVQSGGX |
| | | LVKPGGSLRLSCVASGFTLSSNYMDWVRQAPGKGPQWVAQISTDGSRTYYADA |
| | | VKGRFTISRDTAKNTLYLQMNSLRAEDTAMYYCAKNEYLAPTYWGQGTLVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 394 | scFv_25 | MALPVTALLLPLALLLHAARPGSDIVMTQTPLSLSVSPGETASISCKASQSLLHSN |
| | CAR | GNTYLNWFRQKPGQSPQVLIYQVSNRDTGVPDRFSGSGSGTDFTLRISRVEADD |
| | | AGVYYCGQGIQYPPTFGAGTKVELKGGSSRSSSSGGGGSGGGGEEQLVEFGGD |
| | | LVKPGGSLRLSCVASGFTFSSLSMNWIRQAPGKGLQWVSQISPDGTITGYTDAVK |
| | | GRFTISRDNAKNTLYLQMNSLRDEDTAIYYCAKSRYTDTYPFDYWGPGTLVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 395 | scFv_26 | MALPVTALLLPLALLLHAARPGSSSVLTQPPSVSVSLGQTATISCSGESLSKYYTQ |
| | CAR | WFQQKAGQAPILVIYKDTERPSGIPDRFSGSSSGNTHTLTISGARAEDEADYYCE |
| | | SLVDSDAYMFGSGTQLTVLGGGSSRSSSSGGGGSGGGGEVQLVETGGDLVKPG |
| | | GSLRLSCVGSGFTFSSYWMSWVRQAPGKGLQWIADISDSGSSTDYADAVKGRF |
| | | TISRDNAKNTLYLQMNSLRAEDTAMYYCARGLGMDYWGHGTSLFVSSASEQK |
| | | LISEEDLPSGGGGSGGGGSSPTEPSSKTGNPRHLHVLIGTSVVKIPFTILLFFLLHR |
| | | WCSNKKNAAVMDQEPAGNRTVNSEDSDEQDHQEVSYA |
| 396 | scFv_27 | MALPVTALLLPLALLLHAARPGSQPVLTQPPSVSVSLGQTATISCSGESLSKYYA |
| | CAR | QWFQQKAGQAPVLVIYKDTERPSGIPDRFSGSSSGNTHTLTITGIQAEDEGDYHC |
| | | HSYDENLDGYVFGSGTQLTVLGGGSSRSSSSGGGGSGGGGEVQLVESGGDLVK |
| | | PGGSLRLSCVGSGFTFSSYWMSWVRQAPGKGLQWIADISDSGSSTDYADAVKG |
| | | RFTISRDNAKNTLYLQMNSLRAEDTAMYYCARGLGMDYWGHGTSLFVSSASE |
| | | QKLISEEDLPSGGGGSGGGGSSPTEPSSKTGNPRHLHVLIGTSVVKIPFTILLFFLL |
| | | HRWCSNKKNAAVMDQEPAGNRTVNSEDSDEQDHQEVSYA |
| 397 | scFv_28 | MALPVTALLLPLALLLHAARPGSQSALTQPSSVSVSLGQTATISCSGESLNKYYV |
| | CAR | QWFQQKAGQVPILVIYRDTERPSGIPDRFSGSSSGNTHTLTITGTRAEDEADYYC |
| | | ESLVNDNTYVFGSGTQLTVLGGGSSRSSSSGGGGSGGGGEVQLVETGGDLVKPG |
| | | GSLRLSCVGSGFTFSSYWMSWVRQAPGKGLQWIADISDSGSSTDYADAVKGRF |
| | | TISRDNAKNTLYLQMNSLRAEDTAMYYCARGLGMDYWGHGTSLFVSSASEQK |
| | | LISEEDLPSGGGGSGGGGSSPTEPSSKTGNPRHLHVLIGTSVVKIPFTILLFFLLHR |
| | | WCSNKKNAAVMDQEPAGNRTVNSEDSDEQDHQEVSYA |
| 398 | scFv_29 | MALPVTALLLPLALLLHAARPGSQSVLNQPPSVSVSLGQTATISCSGESLNKYYT |
| | CAR | QWFQQKAGQAPVLVIYKDTERPSGIPDRFSGSSSGNTHTLTISGARAEDEADYY |
| | | CESVDSTDTYVFGSGTQLTILGGGSSRSSSSGGGGSGGGGEVHLVESGGDLVKP |
| | | GGSLRLSCVGSGFTFSSYWMSWVRQAPGKGLQWIADISDSGSSTDYADAVKGR |
| | | FTISRDNAKNTLYLQMNSLRAEDTAMYYCARGLGMDYWGHGTSLFVSSASEQ |
| | | KLISEEDLPSGGGGSGGGGSSPTEPSSKTGNPRHLHVLIGTSVVKIPFTILLFFLLH |
| | | RWCSNKKNAAVMDQEPAGNRTVNSEDSDEQDHQEVSYA |
| 399 | scFv_30 | MALPVTALLLPLALLLHAARPGSQSVLTQPASVSVSLGQTATISCSGESLSKYYT |
| | CAR | QWFQQKAGQAPVLVIYKDTERPSGIPDRFSGSSSGNTHTLTISGAQAEDEADYY |
| | | CQSYDDNVDGYVFGSGTQLTVLGGGSSRSSSSGGGGSGGGGEVQLVESGGDLV |
| | | KPGGSLRLSCVGSGFTFSSYWMSWVRQAPGKGLQWIADISDSGSSTDYADAVK |
| | | GRFTISRDNAKNTLYLQMNSLRAEDTAMYYCARGLGMDYWGHGTSLFVSSAS |
| | | EQKLISEEDLPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 400 | scFv_31 | MALPVTALLLPLALLLHAARPGSQSALTQPSSVSVSLGQTATISCSGESLTKYYV |
| | CAR | QWFQQKAGQAPVLVIYRDTERPSGIPDRFSGSSSGNTHTLTISGARAEDEADYYC |
| | | ESMDGREAYVFGSGTQLTVLGGGSSRSSSSGGGGSGGGGEVQLVESGGDLVKP |
| | | GGSLRLSCVASGFTFSSYWMSWVRQAPGKGLQWIADISDSGSSTDYADAVKGR |
| | | FTISRDNAKNTLYLQMNSLRAEDTAMYYCARGLGMDYWGHGTSLFVSSASEQ |
| | | KLISEEDLPSGGGGSGGGGSSPTEPSSKTGNPRHLHVLIGTSVVKIPFTILLFFLLH |
| | | RWCSNKKNAAVMDQEPAGNRTVNSEDSDEQDHQEVSYA |
| 401 | scFv_32 | MALPVTALLLPLALLLHAARPGSQSVLTQPASVSVSLGQTATISCSGESLSKYYA |
| | CAR | QWFQQKAGQAPVLVIYKDTERPSGIPDRFSGSSSGNTHTLTITGIQAADEADYYC |
| | | QSYDDNLDGYVFGSGTQLTVLGGGSSRSSSSGGGGSGGGGEVQLVESGGDLVK |
| | | PGGSLRLSCVGSGFTFSSYWMSWVRQAPGKGLQWIADISDSGSSTDYADAVKG |
| | | RFTISRDNAKNTLYLQMNSLRAEDTAMYYCARGLGMDYWGHGTSLFVSSASE |
| | | QKLISEEDLPSGGGGSGGGGSSPTEPSSKTGNPRHLHVLIGTSVVKIPFTILLFFLL |
| | | HRWCSNKKNAAVMDQEPAGNRTVNSEDSDEQDHQEVSYA |
| 402 | scFv_33 | MALPVTALLLPLALLLHAARPGSDIVMTQTPLSLSVSPGEPASISCKASQSLLHSD |
| | CAR | GNTYLHWYLQKPGQSPQLLIYSVSNRFTGVSDRFSGSGSGTDFTLRISRVEADDT |
| | | GVYYCGQGTHLPFTFGQGTKLEIKGGSSRSSSSGGGGSGGGGEVQLVESGGDLV |
| | | KPGGSLRLSCAASGFTFSHYDMNWVRQAPGKGLQWVASIAYDGSNTYYTDAV |
| | | KGRFTISRDNAKNILFLLVNSLRAEDTAIYYCARGLVYWGQGTLVTVSSASEQK |
| | | LISEEDLPSGGGGSGGGGSSPTEPSSKTGNPRHLHVLIGTSVVKIPFTILLFFLLHR |
| | | WCSNKKNAAVMDQEPAGNRTVNSEDSDEQDHQEVSYA |
| 403 | scFv_34 | MALPVTALLLPLALLLHAARPGSSYELTQPPSVSAALGQRVTISCTGTNTNIGSG |
| | CAR | YSVQWYQQLPGESPKPIIYGSSNRPSGVPARFSGSKSGSTGTLTITGIQAEDEADY |
| | | HCQSYDDSLDGHAVFGGGTQLTILGGGSSRSSSSGGGGSGGGGEVQLVESGGDL |
| | | VKPGGSLRLSCVASGLTFSDNYMSWIRQVPGKGLQWVSRISTDGRGTSYADAVK |
| | | GRFTISRDNAKNTLYLQMNSLRDEDTAVYYCARRSGIWGFDYWGQGTLVTVSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 404 | scFv_35 | MALPVTALLLPLALLLHAARPGSSSVLTQPPSVSVSLGQTATISCSGESLSEYYAQ |
| | CAR | WFQQKAGQAPVLVIYKDTERASGIPDRFSGSSSGNTHTLTISGARAEDEADYYC |
| | | ESAVNSDGIVFGGGTHLTVLGGGSSRSSSSGGGGSGGGGEVQLVESGGDLVKPA |
| | | GSLRLSCVTSGFTFSSYAMSWVRQAPGKGLQWVGSINSGGSTTSYADAVKGRFT |
| | | ISRDNAKNTVYLQMNSLRAEDTAVYYCARWWDFEYWGQGTLVTVSSASEQKL |
| | | ISEEDLPSGGGGSGGGGSSPTEPSSKTGNPRHLHVLIGTSVVKIPFTILLFFLLHRW |
| | | CSNKKNAAVMDQEPAGNRTVNSEDSDEQDHQEVSYA |
| 405 | scFv_36 | MALPVTALLLPLALLLHAARPGSQSVLTQPTSVSGSLGQRVTISCSGSTNNIGIVG |
| | CAR | ASWYQQLPGKAPKLLVYSDGDRPSGVPDRFSGSNSGNSDTLTITGLQAEDEADY |
| | | YCQSFDTTLDAYVFGSGTQLTVLGGGSSRSSSSGGGGSGGGGEEQLVEFGGGLV |
| | | RPGGSLRLSCVASGFTFSSYGMTWVRQSPGKGLQWVATISDTGSTYYADTVKGR |
| | | FSISRDNAKNTLYLQMNSLRADDTALYYCTKDLGVWGMDNWGPGTSLFVSSAS |
| | | EQKLISEEDLPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 406 | scFv_37 | MALPVTALLLPLALLLHAARPGSSSVLTQPPSVSVSLGQTATISCSGESLNDYYA |
| | CAR | QWFQQKAGQVPVLVIYRDTERPSGIPDRFSGSSSGNTHTLTISGARAEDEADYYC |
| | | ESEVSAGGAVFGEGTHLTVLGGGSSRSSSSGGGGSGGGGEVQLVETGGDLVKPG |
| | | GSLRLSCVASGFTLRDYSMTWVRQTPEKGLQLVAGISTGGSTTYYIDAVKGRFTI |
| | | SRDNAKNTVYLQMNSLRAEDTAMYHCTTTALGYWGQGTLVTVSSASEQKLISE |
| | | EDLPSGGGGSGGGGSSPTEPSSKTGNPRHLHVLIGTSVVKIPFTILLFFLLHRWCS |
| | | NKKNAAVMDQEPAGNRTVNSEDSDEQDHQEVSYA |
| 407 | scFv_38 | MALPVTALLLPLALLLHAARPGSSSVLTQPPSVSVSLGQTATISCSGESLSEYYAQ |
| | CAR | WFQQKAGQAPVLVIYKDTERPSGIPDRFSGSSSGNTHTLTISGARAEDEADYYC |
| | | ASADSSAIPVFGGGTHLTVLGGGSSRSSSSGGGGSGGGGEVQLVESGGDLVKPG |
| | | GSLRLSCVASGFNFGNYAVSWVRQAPGKGLQWVAGISNDGTITGYTDVVEGRFI |
| | | ISRDNAKNTLYLQMNSLRAEDMAIYYCVAGGFLDYWGQGTLVTVSSASEQKLIS |
| | | EEDLPSGGGGSGGGGSSPTEPSSKTGNPRHLHVLIGTSVVKIPFTILLFFLLHRWC |
| | | SNKKNAAVMDQEPAGNRTVNSEDSDEQDHQEVSYA |
| 408 | scFv_39 | MALPVTALLLPLALLLHAARPGSQSVLTQPPSVSGSPGQRVTISCSGTTKNIGIVG |
| | CAR | ASWYQQLPGKAPKLLVHSDGFRPSGVPDRFSASNSGDSDILTITGLQAEDEADY |
| | | YCQSFDTTLDAHVFGSGTQLTVLGGGSSRSSSSGGGGSGGGGEVPLVESGGDLV |
| | | KPGGSLRLSCVASGFTFSSYGMSWVRQSPGKGLQWVAGITYDGSNTYYTDTVR |
| | | GRFTISRDNARNTVYLQMNSLRAEDTAVYYCVASPLYGTYGFDYWGHGTSVFV |
| | | SSASEQKLISEEDLPSGGGGSGGGGSSPTEPSSKTGNPRHLHVLIGTSVVKIPFTIL |
| | | LFFLLHRWCSNKKNAAVMDQEPAGNRTVNSEDSDEQDHQEVSYA |
| 409 | scFv_40 | MALPVTALLLPLALLLHAARPGSQSVLTQPPSVSGSLGQKVTISCTGSSSSVGTH |
| | CAR | YVGWYQIPPGIGPRTVIYNNDYRPSGVPDRFSGSKSGNSATLTISGLQAEDEADY |
| | | YCSSWDDSLGAYVLGSGTQLTILGGGSSRSSSSGGGGSGGGGELTLQESGPGLV |
| | | KPSQTLSLTCAVSGGSVIRGYYWTWIRQRPGRGLEWMGYWAGSTDYNPAFQGR |
| | | IFITAGTAKNQFSLEMTSMTTDDTAIYYCVGSLRGGWLFDNWGQGTLVTVSSAS |
| | | EQKLISEEDLPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 410 | scFv_41 | MALPVTALLLPLALLLHAARPGSQPVLTQPPSVSVSLGQTATISCSGESLSKYYA |
| | CAR | QWFQQKAGQAPVLVIYKDTERPSGIPDRFSGSSSGNTHTLTISGARAEDEADYY |
| | | CESAVSTETVVFGGGTHLTVLGGGSSRSSSSGGGGSGGGGEVQLVETGGDLVMP |
| | | AGSLRLSCVASGFTFSSSVMSWVRQAPGKGLEWVAGINGGGSFAAYGDAVKGR |
| | | FTISRDNAKNTLYLQMNSLRAEDTAMYYCVGGGYMDFWGPGTSLFVSSASEQK |
| | | LISEEDLPSGGGGSGGGGSSPTEPSSKTGNPRHLHVLIGTSVVKIPFTILLFFLLHR |
| | | WCSNKKNAAVMDQEPAGNRTVNSEDSDEQDHQEVSYA |
| 411 | scFv_42 | MALPVTALLLPLALLLHAARPGSQSVLTQPTSVSGSLGQRVTISCSGSTNDIGIIG |
| | CAR | AAWYQQLPGKAPKLLLYSNKNRPSGVPDRFSGSNSGISATLTITGLQAEDEADY |
| | | YCQSVDPALHTYVFGSGTQLTILGGGSSRSSSSGGGGSGGGGEVQLVEIGGDLV |
| | | KPEGSLRLSCVVSGFTFSGYDMNWVRQAPGKGLQWVAYISSDGRRTVYTDAVK |
| | | GRFTISRDNAKNTLYLQMNSLKSEDTAVYYCVKGGWLDFWGQGTLVTVSSASE |
| | | QKLISEEDLPSGGGGSGGGGSSPTEPSSKTGNPRHLHVLIGTSVVKIPFTILLFFLL |
| | | HRWCSNKKNAAVMDQEPAGNRTVNSEDSDEQDHQEVSYA |
| 412 | scFv_43 | MALPVTALLLPLALLLHAARPGSDIVMTQNPLSLAVTPGELATISCRASQSLLHS |
| | CAR | DGKSYLNWYLQKPGQTPRPLIYEASRRFSGVSDRFNGSGSGTDFTLKISRVEAE |
| | | DVGVYYCQQGLHFPPTFGAGTKVELKGGSSRSSSSGGGGSGGGGEVQLVESGG |
| | | DLVKPGGSLRLSCAASGFTFTDYDMSWVRQAPGKGLQWVAAISYDGSSTYYTD |
| | | AVKGRFTISRDNARNTLYLQMSSLRAEDTAMYYCVVSNMNLWGHGTSVFVSSA |
| | | SEQKLISEEDLPSGGGGSGGGGSSPTEPSSKTGNPRHLHVLIGTSVVKIPFTILLFF |
| | | LLHRWCSNKKNAAVMDQEPAGNRTVNSEDSDEQDHQEVSYA |

The exemplary CAR of SEQ ID NOS: 370-412 comprise the amino acid sequence of SPTEPSSKTGNPRHLHVLIGTSVVKIPFTILLFFLLHRWCSNKKNAAVMDQEPAGNRTV NSEDSDEQDHQEVSYA (SEQ ID NO: 266). In some embodiments, the first fusion comprises the sequence of one of SEQ ID NOS: 370-412 with the sequence of SEQ ID NO: 266 replaced by PYEWSNSSDPLLVSVTGNPSNSWPSPTEPSSKTGNPRHLHVLIGTSVVKIPFTILLFFLLH RWCSNKKNAAVMDQEPAGNRTVNSEDSDEQDHQEVSYA (SEQ ID NO: 744). In some embodiments, the first fusion comprises a functional variant of one of the sequences of SEQ ID NOS: 370-412 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of the respective one of SEQ ID NOS: 370-412, where the sequence of SEQ ID NO: 266 is replaced by a functional variant of SEQ ID NO: 744 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 744.

In some embodiments, the CAR comprises an amino acid sequence having about 70%, about 75%, about 80%, about 85%, about 90%, about 91%, about 92%, about 93%, about 94%, about 95%, about 96%, about 97%, about 98%, about 99% identity to the amino acid sequence of SEQ ID NO: 387. In some embodiments, the CAR comprises the amino acid sequence of SEQ ID NO: 387.

In some embodiments, the second fusion protein comprises the amino acid sequence of one of SEQ ID NOS: 620-664. In some embodiments, the second fusion protein comprises the amino acid sequence of a functional variant of the amino acid sequence of one of SEQ ID NOS: 620-664 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of the respective one of SEQ ID NOS: 620-664.

TABLE 8
Exemplary CAR Construct Features and Exemplary Anti-CD20 CARs

| SEQ | | |
|---|---|---|
| ID | | |
| NO: | Name: | Sequence: |
| 262 | scFv KIR | ATGGCCTTACCAGTGACCGCCTTGCTCCTGCCGCTGGCCTTGCTGCTCCACGC |
| | construct | CGCCAGGCCGGGATCCXGCTAGCGAGCAGAAATTAATCTCAGAGGAGGACCT |
| | DNA | GCCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCTCA |
| | | GTGGTCAAAATCCCTTTCACCATCCTCCTCTTCTTTCTCCTTCATCGCTGGTGC |
| | | TCCAACAAAAAAAATGCTGCTGTAATGGACCAAGAGCCTGCAGGGAACAGA |
| | | ACAGTGAACAGCGAGGATTCTGATGAACAAGACCATCAGGAGGTGTCATACG |
| | | CATAA |
| | | (X indicates scFv insertion site) |
| 263 | scFv KIR | MALPVTALLLPLALLLHAARPGSXASEQKLISEEDLPSGGGGSGGGGSSPTEPSS |
| | construct | KTGNPRHLHVLIGTSVVKIPFTILLFFLLHRWCSNKKNAAVMDQEPAGNRTVNS |
| | AA | EDSDEQDHQEVSYA |
| | | (X indicates scFv insertion site) |
| 264 | Myc tag | GAGCAGAAATTAATCTCAGAGGAGGACCTG |
| 746 | Myc tag | EQKLISEEDL |
| 755 | 6X His tag | HHHHHH |
| 756 | Flag tag | DYKDDDDK |
| 757 | Strep Tag II | WSHPQFEK |
| 758 | SBP | MDEKTTGWRGGHVVEGLAGELEQLRARLEHHPQGQREP |
| | (Streptavidin | |
| | Binding | |
| | Protein | |
| 759 | HA | YPYDVPDYA |
| | (hemaggltinin) | |
| 260 | Linker | SGGGGSGGGGS |
| 811 | Linker | SGGGGSGGGGSGGGGSGGGGSGGGGS |
| 821 | (G4S)n | GGTGGCGGAGGTTCT |
| | Linker | |
| | DNA 1 | |
| 822 | (G4S)n | GGAGGTGGGGGTTCC |
| | Linker | |
| | DNA 2 | |
| 823 | (G4S)n | GGAGGCGGCGGCAGC |
| | Linker | |
| | DNA 3 | |
| 824 | (G4S)n | GGCGGGGGAGGGTCC |
| | Linker | |
| | DNA 4 | |
| 825 | (G4S)n | GGAGGGGGTGGTTCT |
| | Linker | |
| | DNA 5 | |
| 826 | (G4S)n | GGTGGAGGAGGATCG |
| | Linker | |
| | DNA 6 | |
| 827 | (G4S)n | GGAGGCGGTGGCAGC |
| | Linker | |
| | DNA 7 | |
| 828 | (GSGGS)n | GGCAGCGGCGGCAGC |
| | Linker | |
| 829 | (GGGS)n | GGCGGTGGTTCC |
| | Linker | |
| 830 | Linker A | GGGSSRSSSSGGGGSGGGG |
| 831 | Linker A | GGCGGTGGTTCCTCTAGATCTTCCTCCTCTGGTGGCGGTGGCTCGGGCGGTG |
| | DNA | GTGGG |
| 832 | Whitlow | GSTSGSGKPGSGEGSTKG |
| | Linker | |
| 833 | Whitlow | GGCAGCACATCCGGTTCCGGGAAGCCCGGCTCCGGAGAGGGCAGCACCAAG |
| | Linker | GGG |
| | DNA | |
| 265 | TM/KIR2D | CCCACTGAACCAAGCTCCAAAACCGGTAACCCCAGACACCTGCATGTTCTGA |
| | S2 domains | TTGGGACCTCAGTGGTCAAAATCCCTTTCACCATCCTCCTCTTCTTTCTCCTTC |
| | | ATCGCTGGTGCTCCAACAAAAAAAATGCTGCTGTAATGGACCAAGAGCCTGC |
| | | AGGGAACAGAACAGTGAACAGCGAGGATTCTGATGAACAAGACCATCAGGA |
| | | GGTGTCATACGCA |
| 266 | TM/KIR2D | SPTEPSSKTGNPRHLHVLIGTSVVKIPFTILLFFLLHRWCSNKKNAAVMDQEPAG |
| | S2 (short | NRTVNSEDSDEQDHQEVSYA |
| | stem) | |
| | domains | |
| 740 | KIR Short | SPTEPSSKTGNPRHLH |
| | Extracellular | |
| | portion | |
| | (short stem | |
| | domain) | |
| 741 | KIR Long | PYEWSNSSDPLLVSVTGNPSNSWPSPTEPSSKTGNPRHLH |
| | Extracellular | |
| | portion | |
| | (long stem | |
| | domain) | |
| 742 | KIR | VLIGTSVVKIPFTILLFFLLH |
| | Transmembrane | |
| | domain | |
| 743 | KIR | RWCSNKKNAAVMDQEPAGNRTVNSEDSDEQDHQEVSYA |
| | Intracellular | |
| | Domain | |
| 744 | TM/KIR2D | PYEWSNSSDPLLVSVTGNPSNSWPSPTEPSSKTGNPRHLHVLIGTSVVKIPFTILL |
| | S2 Long | FFLLHRWCSNKKNAAVMDQEPAGNRTVNSEDSDEQDHQEVSYA |
| | stem | |
| | domain | |
| 357 | CD8 | MALPVTALLLPLALLLHAARP |
| | Leader | |
| | Sequence | |
| 674 | GM-CSF | MWLQNLLLLGAVVCSIS |
| | Leader | |
| | Sequence | |
| 675 | IgK Leader | METPAQLLFLLLLWLPDTTG |
| | Sequence | |
| 676 | IL-22 | MAALQKSVSSFLMGTLATSCLLLLALLVQGGAA |
| | Leader | |
| | Sequence | |
| 754 | IgK Leader | METPAQLLFLLLLWLPDTTG |
| 620 | scFv_44 | MALPVTALLLPLALLLHAARPGSMDFQVQIISFLLISASVIMSRGQIVLSQSPAILS |
| | CAR | ASPGEKVTMTCRASSSVSYIHWFQQKPGSSPKPWIYATSNLASGVPVRFSGSGSG |
| | | TSYSLTISRVEAEDAATYYCQQWTSNPPTFGGGTKLEIKGGGSSRSSSSGGGGSG |
| | | GGGMGWSLILLFLVAVATRVLSQVQLQQPGAELVKPGASVKMSCKASGYTFTSY |
| | | NMHWVKQTPGRGLEWIGAIYPGNGDTSYNQKFKGKATLTADKSSSTAYMQLSS |
| | | LTSEDSAVYYCARSTYYGGDWYFNVWGAGTTVTVSAASEQKLISEEDLPSGGG |
| | | GSGGGGSSPTEPSSKTGNPRHLHVLIGTSVVKIPFTILLFFLLHRWCSNKKNAAV |
| | | MDQEPAGNRTVNSEDSDEQDHQEVSYA |
| 621 | scFv_45 | MALPVTALLLPLALLLHAARPGSEIVLTQSPATLSLSPGERATLSCRASQSVSSYL |
| | CAR | AWYQQKPGQAPRLLIYDASNRATGIPARFSGSGSGTDFTLTISSLEPEDFAVYYCQ |
| | | QRSNWPITFGQGTRLEIKGGGSSRSSSSGGGGSGGGGEVQLVESGGGLVQPGRS |
| | | LRLSCAASGFTFNDYAMHWVRQAPGKGLEWVSTISWNSGSIGYADSVKGRFTIS |
| | | RDNAKKSLYLQMNSLRAEDTALYYCAKDIQYGNYYYGMDVWGQGTTVTVSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 622 | scFv_46 | MALPVTALLLPLALLLHAARPGSEIVLTQSPATLSLSPGERATLSCRASQSVSSYL |
| | CAR | AWYQQKPGQAPRLLIYDASNRATGIPARFSGSGSGTDFTLTISSLEPEDFAVYYCQ |
| | | QRSNWPITFGQGTRLEIKGGGSSRSSSSGGGGSGGGGEVQLVESGGGLVQPDRS |
| | | LRLSCAASGFTFHDYAMHWVRQAPGKGLEWVSTISWNSGTIGYADSVKGRFTIS |
| | | RDNAKNSLYLQMNSLRAEDTALYYCAKDIQYGNYYYGMDVWGQGTTVTVSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 623 | scFv_47 | MALPVTALLLPLALLLHAARPGSEIVLTQSPATLSLSPGERATLSCRASQSVSSYL |
| | CAR | AWYQQKPGQAPRLLIYDASNRATGIPARFSGSGSGTDFTLTISSLEPEDFAVYYCQ |
| | | QRSDWPLTFGGGTKVEIKGGGSSRSSSSGGGGSGGGGEVQLVQSGGGLVHPGGS |
| | | LRLSCTGSGFTFSYHAMHWVRQAPGKGLEWVSIIGTGGVTYYADSVKGRFTISR |
| | | DNVKNSLYLQMNSLRAEDMAVYYCARDYYGAGSFYDGLYGMDVWGQGTTVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 624 | scFv_48 | MALPVTALLLPLALLLHAARPGSDIVMTQTPLSLPVTPGEPASISCRSSKSLLHSN |
| | CAR | GITYLYWYLQKPGQSPQLLIYQMSNLVSGVPDRFSGSGSGTDFTLKISRVEAEDV |
| | | GVYYCAQNLELPYTFGGGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKV |
| | | YACEVTHQGLSSPVTKSFNRGECGGGSSRSSSSGGGGSGGGGQVQLVQSGAEV |
| | | KKPGSSVKVSCKASGYAFSYSWINWVRQAPGQGLEWMGRIFPGDGDTDYNGK |
| | | FKGRVTITADKSTSTAYMELSSLRSEDTAVYYCARNVFDGYWLVYWGQGTLVT |
| | | VSSASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKS |
| | | LSLSPGASEQKLISEEDLPSGGGGSGGGGSSPTEPSSKTGNPRHLHVLIGTSVVKI |
| | | PFTILLFFLLHRWCSNKKNAAVMDQEPAGNRTVNSEDSDEQDHQEVSYA |
| 625 | scFv_49 | MALPVTALLLPLALLLHAARPGSQIVLSQSPAILSASPGEKVTMTCRASSSVSYIH |
| | CAR | WFQQKPGSSPKPWIYATSNLASGVPVRFSGSGSGTSYSLTISRVEAEDAATYYCQ |
| | | QWTSNPPTFGGGTKLEIKGGGSSRSSSSGGGGSGGGGQVQLQQPGAELVKAGA |
| | | SVKMSCKASGYTFTSYNMHWVKQTPGRGLEWIGAIYPGNGDTSYNQKFKGKA |
| | | TLTADKSSSTAYMQLSSLTSEDSAVYYCARSTYYGGDTYFNVWGAGTTVTVSA |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 626 | scFv_50.1 | MALPVTALLLPLALLLHAARPGSQIVLSQSPAIITASPGEKVTMTCRASTSASYIH |
| | CAR | WFQQKPTSSPKPWIYATSNLASGVPSRFSGSGSGTTYSMTISSLEAEDAATYYCQ |
| | | QWTSNPPTFGGGTKLEIKGGGSSRSSSSGGGGSGGGGQVQLQQPGAELKKPGA |
| | | SVKVSCKASGYTFTSYNMHWVKQTPGRGLEWTGAIYPGNGDTSYNQKFKGKT |
| | | TLTADKSSSTAYMELSSLRSEDTAVYYCARSTYYGGDWYFNVWGAGTTVTVSA |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 627 | scFv_50.2 | MALPVTALLLPLALLLHAARPGSQIVLSQSPAIITASPGEKVTMTCRASTSVSYIH |
| | CAR | WFQQKPTSSPKPWIYATSNLASGVPSRFSGSGSGTTYSMTISSLEAEDAATYYCQ |
| | | QWTSNPPTFGGGTKLEIKGGGSSRSSSSGGGGSGGGGQVQLQQPGAELKKPGA |
| | | SVKVSCKASGYTFTSYNMHWVKQTPGRGLEWIGAIYPGNGDTSYNQKFKGKT |
| | | TLTADKSSSTAYMELSSLRSEDTAVYYCARSTYYGGDWYFNVWGAGTTVTVSA |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 628 | scFv_51 | MALPVTALLLPLALLLHAARPGSDIVLTQSPAILSASPGEKVTMTCRASSSVNYM |
| | CAR | DWYQKKPGSSPKPWIYATSNLASGVPARFSGSGSGTSYSLTISRVEAEDAATYYC |
| | | QQWSFNPPTFGGGTKLEIKGGGSSRSSSSGGGGSGGGGEVQLQQSGAELVKPGA |
| | | SVKMSCKASGYTFTSYNMHWVKQTPGQGLEWIGAIYPGNGDTSYNQKFKGKA |
| | | TLTADKSSSTAYMQLSSLTSEDSADYYCARSNYYGSSYWFFDVWGAGTTVTVSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 629 | scFv_51.1 | MALPVTALLLPLALLLHAARPGSDIVLTQSPAIITASPGEKVTMTCRASSSVNYM |
| | CAR | DWYQKKPGSSPKPWIYATSNLASGVPSRFSGSGSGTTYSMTISSLEAEDAATYYC |
| | | QQWSFNPPTFGGGTKLEIKGGGSSRSSSSGGGGSGGGGEVQLQQSGAELKKPG |
| | | ASVKVSCKASGYTFTSYNMHWVKQTPGQGLEWIGAIYPGNGDTSYNQKFKGK |
| | | TTLTADKSSSTAYMELSSLRSEDTAVYYCARSNYYGSSYWFFDVWGTGTTVTVS |
| | | SASEQKLISEEDLPSGGGGSGGGGSSPTEPSSKTGNPRHLHVLIGTSVVKIPFTILL |
| | | FFLLHRWCSNKKNAAVMDQEPAGNRTVNSEDSDEQDHQEVSYA |
| 630 | scFv_52 | MALPVTALLLPLALLLHAARPGSQIVLSQSPTILSASPGEKVTMTCRASSSVSYM |
| | CAR | DWYQQKPGSSPKPWIYATSNLASGVPARFSGSGSGTSYSLTISRVEAEDAATYYC |
| | | QQWISNPPTFGAGTKLELGGGSSRSSSSGGGGSGGGGQVQLQQPGAELVKPGAS |
| | | VKMSCKTSGYTFTSYNVHWVKQTPGQGLEWIGAIYPGNGDTSFNQKFKGKATL |
| | | TADKSSSTVYMQLSSLTSEDSAVYYCARSNYYGSSYVWFFDVWGAGTTGTGSS |
| | | ASEQKLISEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 631 | scFv_53 | MALPVTALLLPLALLLHAARPGSDIVMTQTPLSSPVTLGQPASISCRSSQSLVYSD |
| | CAR | GNTYLSWLQQRPGQPPRLLIYKISNRFSGVPDRFSGSGAGTDFTLKISTRQVFEP |
| | | ALETDFVGGGVGYTYKCVVEQIAKGGGSSRSSSSGGGGSGGGGEVQLVQSGAE |
| | | VKKPGESLKISCKGSGYSFTSYWIGWVRQMPGKGLEWMGIIYPGDSDTRYRPSF |
| | | QGQVTISADKSITTAYLQWSSLKASDTAMYYCARHPSYGSGSPNFDYWGQGTL |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 632 | scFv_54 | MALPVTALLLPLALLLHAARPGSEIVMTQSPATLSVSPGERATLSCRASQSVSSN |
| | CAR | LAWYQQKPGQAPRLLISGASTRATGIPARFSGSGSGTEFTLTISSLQSEDFAVYYC |
| | | HQYNDWSLTFGGGTKVEIKGGGSSRSSSSGGGGSGGGGSCKGSGYSFTSYWIG |
| | | WVRQMPGKGLEWMGIIYPGDSDTRYSPSFQGQVTISADKSISTAYLQWSSLKAS |
| | | DTAMYYCARMGDFWSGYYTRGMDVWGQGTTVTVSSASEQKLISEEDLPSGGG |
| | | GSGGGGSSPTEPSSKTGNPRHLHVLIGTSVVKIPFTILLFFLLHRWCSNKKNAAV |
| | | MDQEPAGNRTVNSEDSDEQDHQEVSYA |
| 633 | scFv_55 | MALPVTALLLPLALLLHAARPGSDIVMTQTPLSSPVTLGQPASISCRSSQRLVHS |
| | CAR | DGHTYLSWLQQRPGQPPRLLISKVSNRFSGVPDRFSGSGAGTDFTLKISRVEAED |
| | | VGIYYCMQATQFPLTFGGGTKVEIKGGGSSRSSSSGGGGSGGGGEVQLVQSGAE |
| | | VKKPGESLKISCKGSGYSFTNYWIVWVRQMPGKGLEWMGIIYPGDSDTRYSPSF |
| | | QGQVTISADKSISTAYLQWSSLKASDTAMYYCARHGDYYYYYGMDVWGQGTT |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 634 | scFv_56 | MALPVTALLLPLALLLHAARPGSDIVMTQTPLSSPVTLGQPASISCRSSQSLVYSD |
| | CAR | GNTYLSWLQQRPGQPPRLLFYKISNRFSGVPDRFSGSGAGTDFTLKISRVEAEDV |
| | | GVYYCVQATQFPLTFGGGTKVEIKGGGSSRSSSSGGGGSGGGGEVQLVQSGAEV |
| | | KKPGESLKISCKGSGYSFTSYWIGWVRQMSGKGLEWMGIIYPGDSDTRYSPSFQ |
| | | GQVTISADKSISTAYLQWSSLKASDTAIYYCARHPSYGSGSPNFDYWGQGTLVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 635 | scFv_57 | MALPVTALLLPLALLLHAARPGSDVVMTQSPLSLSVTLGQPASISCRSSQSLVYN |
| | CAR | DGDTYLNWFQQRPGQSPRLLIYKVSNWDSGVPDRFSGSGSGTDFTLKVSRVEA |
| | | EDVGVYYCMQGTHWPLTFGGGTKVEIKGGGSSRSSSSGGGGSGGGGEVQLVQS |
| | | GAEVKKPGESLKISCKGSGYSFTNYWIGWVRQMPGKGLEWMGIIYPGDSDTRY |
| | | SPSFQGQVTISADKSISTAYLQWSSLKASDTAMYYCARQGDYYHYSGMDVWG |
| | | QGTTVTVSSASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDEQDHQEVSYA |
| 636 | scFv_58 | MALPVTALLLPLALLLHAARPGSDIVMTQTPLSSPVTLGQPASISCRSSQSLVHSD |
| | CAR | GKTYLSWLQQRPGQPPRLLIYKSSNRFSGVPDRFSGSGAGTDFTLKISRVEAEDV |
| | | GVYYCVQATQFPITFGQGTRLEIKGGGSSRSSSSGGGGSGGGGEVQLVQSGAEV |
| | | KKPGESLKISCKGSGYSFTSYWIGWVRQMPGKGLEWMGIIYPGDSDTRYSPSFQ |
| | | GQVTISADKSISTAYLQWSSLKASDTAMYYCARLGDYYNYYGMDVWGQGTTV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 637 | scFv_59 | MALPVTALLLPLALLLHAARPGSDIVMTQTPLSSPVTLGQPASISCRSSQSLVHSD |
| | CAR | GNTYLSWLQQRPGQPPRLLIYKISNRFSGVPDRFSGSGAGTDFTLKISRVEAEDV |
| | | GVYYCMQAIQFPITFGQGTRLEIKGGGSSRSSSSGGGGSGGGGEVQLVQSGAEV |
| | | KKPGESLKISCKGSGYSFTSYWIGWVRQMPGKGLEWMGIIYPGDSDTRYSPSFQ |
| | | GQVTISADKSITTAYLQWSSLKASDTAMYYCARHPSYGSGSPNFDYWGQGTLV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 638 | scFv_60 | MALPVTALLLPLALLLHAARPGSDIVMTQTPLSSPVTLGQPASISCRSSQSLVYSD |
| | CAR | GNTYLSWLQQRPGQPPRLLIYKISNRFSGVPDRFSGSGAGTDFTLKISTRQVFEP |
| | | ALETDFVGGGVGYTYKCVVEQIAKGGGSSRSSSSGGGGSGGGGEVQLVQSGAE |
| | | VKKPGESLKISCKGSGYSFTSYWIGWVRQMPGKGLEWMGIIYPGDSDTRYSPSF |
| | | QGQVTISADKSITTAYLQWSSLKASDTAMYYCARHPSYGSGSPNFDYWGQGTL |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 639 | scFv_61 | MALPVTALLLPLALLLHAARPGSDVVMTQSPLSLPVTLGQPASISCRSSQSLVYS |
| | CAR | DGNAYLNWFQQRPGQSPRRLIYKVSNWDSGVPDRFSGSGSGTDFTLKIRRVEAE |
| | | DVGAYYCMQGIHCTFGPGTKVHIKGGGSSRSSSSGGGGSGGGGEVQLVQSGAE |
| | | VKKPGESLKISCKGSGYSFTSYWIGWVRQMPGKGLEWMGIIYPGDSDTRYSPSF |
| | | QGQVTISADKSISTAYLQWSSLKASDTAMYYCARHGDYSNIDAFDIWGQGTMV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 640 | scFv_62 | MALPVTALLLPLALLLHAARPGSDIVMTQTPLSSPVTLGQPASISCRSSQSLVHSD |
| | CAR | GNTFLSWLQQRPGQPPRLLIYKISNRFSGVPDRFSGSGAGTDFTLKISRVEAEDV |
| | | GVYYCMQLTQFPLTFGGGTKVEIRGGGSSRSSSSGGGGSGGGGEVQLVQSGAEV |
| | | KKPGESLKISCKGSGYSFTSYWIGWVRQMPGKGLEWMGIIYPGDSDTRYSPSFQ |
| | | GQVTISADKSISTAYLQWSSLKASDTAMYYCARTGTTDYYSGMDVWGQGTTVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 641 | scFv_63 | MALPVTALLLPLALLLHAARPGSDVVMTQSPLSLPVTLGQPASISCRSSQSLVYS |
| | CAR | DGNTYLNWFQQRPGQSPRRLIYKVSNWDSGVPDRFSGSGSGTDFTLKISRVEAE |
| | | DVGVYYCMQGTHWPSITFGQGTRLEIKGGGSSRSSSSGGGGSGGGGEVQLVQS |
| | | GAEVKKPGESLKISCKGSGYSFTSYWIGWVRQMPGKGLEWMGIIYPGDSDTRY |
| | | SPSFQGQVTISADKSISTAYLQWSSLKASDTAMYYCARHGDFWSGYYSFDYWG |
| | | QGTLVTVSSASEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQDHQEVSYA |
| 642 | scFv_64 | MALPVTALLLPLALLLHAARPGSDIVMTQTPHSSPVTLGQPASISCRSSQSLVSRD |
| | CAR | GNTYLSWLQQRPGQPPRLLIYKISNRFSGVPNRFSGSGAGTDFTLKISRVKAEDV |
| | | GVYYCMQATQFPLTFGQGTRLEIKGGGSSRSSSSGGGGSGGGGEVQLVQSGAEV |
| | | KKPGESLKISCKGSGYSFTSYWIGWVRQMPGKGLEWMGIIYPGDSDTRYSPSFQ |
| | | GQVTISADKSISTAYLQWSSLKASDTAMYYCARQGDFWSGYGGMDVWGQGTT |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 643 | scFv_65 | MALPVTALLLPLALLLHAARPGSDIVMTQTPLSSPVTLGQPASISCRSSQSLVSRD |
| | CAR | GNTYLSWLQQRPGQPPRLLIYKISNRFSGVPDRFSGSGAGTDFTLKISRVEVEDV |
| | | GVYYCMQATQFPITFGQGTRLEIKGGGSSRSSSSGGGGSGGGGEVQLVQSGAEV |
| | | KKPGESLKISCKGSGYSFTSYWIGWVRQMPGKGLEWMGIIYPGDSDTKYSPSFQ |
| | | GQVTISADKSISTAYLQWSSLKASDTAMYYCARQGDFWSGNGGMDVWGQGTT |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 644 | scFv_66 | MALPVTALLLPLALLLHAARPGSDIVMTQTPLSSPVTLGQPASISCRSSQSLVHSD |
| | CAR | GNTFLSWLQQRPGQPPRLLIYKISNRFSGVPDRFSGSGAGTDFTLKISRVEAEDV |
| | | GVYYCMQGTQFPLTFGGGTKVEIKGGGSSRSSSSGGGGSGGGGEVQLVQSGAE |
| | | VKKPGESLKISCKGSGYSFTNYWIGWVRQMPGKGLEWMGIIYPGDSDTRYSPSF |
| | | QGQVTISADKSISTAYLQWSSLKASDTAMYYCARHGDYYASESSAFDIWGQGT |
| | | MVTVSSASEQKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDHQEVSYA |
| 645 | scFv_67 | MALPVTALLLPLALLLHAARPGSDIVMTQSPLSSPVTLGQPASISCRYSQSLVHR |
| | CAR | DGNTYLSWLQQRPGQPPRLVIHKISNRFSGVPDRFSGSGAGTDFTLKISRVEAED |
| | | VGFYYCMQTTQFPLTFGGGTKVEIKGGGSSRSSSSGGGGSGGGGEVQLVQSGAE |
| | | VKKPGESLKISCKGSGYSFTSYWIDWVRQMPGKGLEWMGSIYPGDSDTRYSPSF |
| | | QGQVTISADKSISTAYLQWSSLKASDTAMYYCARQGASGYYYGMDVWGQGTT |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 646 | scFv_68 | MALPVTALLLPLALLLHAARPGSDIVMTQTPLSSPVTLGQPASISCRSSQSLVHSD |
| | CAR | GNTYLSWLQQRPGQPPRLLIYKISNRFSGVPDRFSGSGAGTDFTLKISRVEAEDV |
| | | GVYYCMQATQFPLTFGGGTKVEIKGGGSSRSSSSGGGGSGGGGQVQLVQSGAE |
| | | VKKPGASVKVSCTASGYSFSSYGISWVRQAPGQGLEWMGWISAYNGHTRYAQ |
| | | KLQGRVTMTSDTSTSTAYMELRSLRSDDTAVYYCARASSWYFDCWGQGTLVTV |
| | | SSASEQKLISEEDLPSGGGGSGGGGSSPTEPSSKTGNPRHLHVLIGTSVVKIPFTIL |
| | | LFFLLHRWCSNKKNAAVMDQEPAGNRTVNSEDSDEQDHQEVSYA |
| 647 | scFv_69 | MALPVTALLLPLALLLHAARPGSEIVMTQTPLSSPVTLGQPASISCKSSQSLVHSD |
| | CAR | GNTYLSWLQQRPGQPPRLLIYKISNRFSGVPDRFSGSGAGTDFTLKISRVEAEDV |
| | | GVYYCMQTTYFPLTFGGGTKVEIKGGGSSRSSSSGGGGSGGGGQVQLVQSGAE |
| | | VKKPGASVKVSCKASGYTFTSYGISWVRQAPGQGLEWMGWINAYNGNTNYAQ |
| | | KLRGRVTMTTDTFTSTADMELRSLRSDDTAVYYCARASTAMGDYWGQGTLVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 648 | scFv_70 | MALPVTALLLPLALLLHAARPGSDIVMTQTPLSSPVTLGQPASISCRSSQSLVYSD |
| | CAR | GNTYLSWLHQRPGQPPRLLIYKISNRFSGVPDRFSGSGAGTDFTLKISRVEAEDV |
| | | GLYYCMQTTQFPLTFGGGTKVKIKGGGSSRSSSSGGGGSGGGGEVQLVQSGAE |
| | | VKKPGESLKISCKGSGYSFTNYWIAWVRQMPGKGLEWMGIIFPGDSDTRYSPAF |
| | | QGQVTISADKSISTAYLHWSSLKASDTAMYYCARHRDYTSGGPDAFDVWGQGT |
| | | MVTVSSASEQKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDHQEVSYA |
| 649 | scFv_71 | MALPVTALLLPLALLLHAARPGSDIVMTQTPLSSPVTLGQPASISCRSSQSLVHSD |
| | CAR | GNTYLSWLQQRPGQPPRLLIYKLSNRFSGIPDSRRFVSGEASEGDAVGGTVDYF |
| | | YTCLKMQIATQFPLTFGGGTKVEIKGGGSSRSSSSGGGGSGGGGEVQLVQSGAE |
| | | VKKPGESLKISCKGSGYSFTSYWIAWVRQMPGKGLEWMGIIYPGDSDTRYSPSF |
| | | QGQVTISADKSISTAYLQWSNLKASDTAMYYCARTGSYYNYCGMDVWGQGTT |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 650 | scFv_72 | MALPVTALLLPLALLLHAARPGSDIVMTQTPLSSPVTLGQPASISCRSSQSLVYR |
| | CAR | DGNTYLSWLQQRPGQPPRLLIYKISNRFSGVPDRFSGSGAGTDFTLKISRVEAED |
| | | VGVYYCMQATQFPLTFGGGTKVEIKGGGSSRSSSSGGGGSGGGGEVQLVQSGA |
| | | EVKKPGESLKISCKGSGYSFTNYWIAWVRQMPGKGLEWMGIIYPGDSDTRYSPS |
| | | FQGQVTISADKSISTAYLQWSSLKASDTAMYYCTRQGDYYDSSGPDYWGQGTL |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 651 | scFv_73 | MALPVTALLLPLALLLHAARPGSKIVMTQTPLSSPVTLGQPASISCRSSQSLVHSD |
| | CAR | GNTYLSWFQQRPGQPPRLLINKISNRFSGVPDRFSGSGAGTDFTLKISRVEAEDV |
| | | GVYYCMQATQFPLTFGGGTKVEIGGGSSRSSSSGGGGSGGGGQVQLVESAGGV |
| | | VQPGRSLRLSCAASGFTFSSYGMHWVRQAPGKGLEWVAVIWHDGSKKYYEDS |
| | | VKGRFTISRDNSKNTLYLQMNSLRAEDTAVYYCARNWFFDYWGQGTLVTVSSA |
| | | SEQKLISEEDLPSGGGGSGGGGSSPTEPSSKTGNPRHLHVLIGTSVVKIPFTILLFF |
| | | LLHRWCSNKKNAAVMDQEPAGNRTVNSEDSDEQDHQEVSYA |
| 652 | scFv_74 | MALPVTALLLPLALLLHAARPGSSSELTQDPAVSVALGQTVRITCQGDSLRSYYA |
| | CAR | SWYQQKPGQAPVLVIYGKNNRPSGIPARFSGSDSGNTASLTITGAQAEDEADYY |
| | | CNSRDSSGNHVVFGGGTKLTVLGGGSSRSSSSGGGGSGGGGQVQLVESGGGLV |
| | | KPGGSLRLSCAASGFTFSDYYMSWIRQAPGKGLEWVSYISSSGTTIYYADSVKG |
| | | RFTISRDNAKNSLYLQMNSLRAEDTAVYYCARDLYYGGNSYYFDYWGQGTLVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 653 | scFv_75 | MALPVTALLLPLALLLHAARPGSDIVMTQTPLSSPVTLGQPASISCRSSQSLVHSD |
| | CAR | GNTYLSWLQQRPGQPPRLLLYKNSNRFSGVPDRFSGSGAGTDFTLKISRVEAED |
| | | VGVYYCMQATQFPLTFGGGTKVEIKGGGSSRSSSSGGGGSGGGGEVQLVQSGA |
| | | EVKKPGESLKISCKGSGYSFTSYWGIIGIWYVPRGQDMSPDGTKRGYLSEPWSM |
| | | FQGQVTISADKSIRTAYLQWSSLKASDTAMYYCARIGDHYHYNGMDVWGQGT |
| | | TVTVSSASEQKLISEEDLPSGGGGSGGGGSSPTEPSSKTGNPRHLHVLIGTSVVKI |
| | | PFTILLFFLLHRWCSNKKNAAVMDQEPAGNRTVNSEDSDEQDHQEVSYA |
| 654 | scFv_76 | MALPVTALLLPLALLLHAARPGSDIVMTQTPPSSPVTRGQPASISCRSSQSLVHSD |
| | CAR | GKTYLSWLQQRPGQPPRLLIYKISNRFSGVPGRFSGSGAGTDFTLKISRVEAEDV |
| | | GVYYCMQATQFPLTFGGGTKVEIKGGGSSRSSSSGGGGSGGGGEVQLVQSGAE |
| | | VKKPGESLKISCKGSGYSFSSYWINWVRQMPGKGLEWMGIIYPGDSDTRYSPSF |
| | | QGQVTISADKSISTAYLQWRSLKASDTAIYYCARVGDYYSYYGMDVWGQGTTV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 655 | scFv_77 | MALPVTALLLPLALLLHAARPGSDIVMTQTPLSSPVTLGQPASISCRSRQSLLHSD |
| | CAR | GNTYLSWLQQRPGQPPRLLIYKLSNRVSGVPDRFSGSGAGTDFTLKISRVEAEDV |
| | | GVYYCMQSTQFPLTFGGGTKVKIKGGGSSRSSSSGGGGSGGGGEVQLVQSGAE |
| | | VKKPGESLKISCKGSGYSFTNYWINWVRQMPGKGLEWMGIIYPGDSDTRYSPSF |
| | | QGQVTISADKSINTAYLQWSSLKASDTAMYYCARQGGHYYYSGMDVWGQGTT |
| | | VTVSSASEQKLISEEDLPSGGGGSGGGGSSPTEPSSKTGNPRHLHVLIGTSVVKIP |
| | | FTILLFFLLHRWCSNKKNAAVMDQEPAGNRTVNSEDSDEQDHQEVSYA |
| 656 | scFv_78 | MALPVTALLLPLALLLHAARPGSDIVMTQTPLSSPVTLGQPASISCRSSQSLVHSD |
| | CAR | RNTYLSWLQQRPGQPPRLLIYKVSNRFSGVPERFSGSGTGTDFTLKISRVEAEDV |
| | | GVYYCVQETLFPITIGQGTRLEIKGGGSSRSSSSGGGGSGGGGEVQLVQSGAEV |
| | | KKPGESLKISCKGSRNSFTNYWIGWVRQMPGKGLEWMGIIYPGDSDTRYSPSFQ |
| | | GQVTISADKSISTAYLQWSSLKASDTAMYYCARTGSYSYYYGMDVWGQGTTV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 657 | scFv_79 | MALPVTALLLPLALLLHAARPGSDIVMTQTPLSSPVTLGQPASISCRSSQSLVHSD |
| | CAR | GNTFLSWLQQRPGQPPRLLIYKISNRFSGVPDRFSGSGAGTDFTLKISRVEAEDV |
| | | GIYYCMQATQFPLTFGGGTKVEIKGGGSSRSSSSGGGGSGGGGGVQLVQSGAEV |
| | | KKPGESLKISCKGSGYSFTSYWIGWVRQMPGKGLEWMGIIYPGDSDTRYSPSFQ |
| | | GQVIISADKSISTAYLQWSSLKASDTAMYYCARTGDYYSYHGMDVWGQGTTVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 658 | scFv_80 | MALPVTALLLPLALLLHAARPGSDIVLTQTPLSSPVTLGQPASISCRSSQSLVHSD |
| | CAR | GHTYLSWLQQRPGQPPRLLIYKISNRFSGVPDRFSGSGAGTDFTLKISRVGAEDV |
| | | GVYYCMQATQFPLTFGGGTKVEIKGGGSSRSSSSGGGGSGGGGEVQLVQSGAE |
| | | VKKSGESLKISCKGSGYSFTNFWIGWVRQMPGKGLEWMGIIYPGDSDTRYSPSF |
| | | QGQVTISADKSISTTYLQWSSLKASDTAMYYCARHPPYSGSYYADAFDIWGQG |
| | | TMVTVSSASEQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQDHQEVSYA |
| 659 | scFv_81 | MALPVTALLLPLALLLHAARPGSDIVMTQTPLSSPVTLGQPASISCRSSQSLVSSD |
| | CAR | GNTYLSWLQQRPGQPPRLLIYKISNLFSGVPDRFSGSGAGTDFTLKISRVEAEDV |
| | | GLYYCMQATQFPLTFGGGTKVEIKGGGSSRSSSSGGGGSGGGGEVQLVQSGAEV |
| | | KKPGESLKISCKGSGYSFTSFWIGWVRQMPGKGLEWMGIIYPGDSDTRYSPSFR |
| | | GLVTISADKSISTAYLHWSSLKASDTAIFYCARHPPYSGSYYADAFDIWGQGTMV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 660 | scFv_82 | MALPVTALLLPLALLLHAARPGSDQIPVAMSTIQSSCPRLSSSSQPSVLTVLHGSD |
| | CAR | GNTFLSWLQQRPGQPPRLLIYKISNRFSGVPDRFSGSGAGTDFTLKISRVEAEDV |
| | | GVYYCIQATQFPLTFGGGTKVEIKGGGSSRSSSSGGGGSGGGGEVQLVQSGAEV |
| | | KKPGESLKISCKGSGYSFTSYWIGWVRQMPGKGLEWMGIIYPGDSDTRYSPSFQ |
| | | GQVTISADKSISTAYLQWSSLKASDTAMYYCARTGDYHNYYGMDVWGQGTTV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 661 | scFv_83 | MALPVTALLLPLALLLHAARPGSDVVMTQSPLSLPVTLGQPASISCRSRQSLVYS |
| | CAR | DGSTYLNWFQQRPGQSPRRLIYKVSNWDSGVPDRFSASGSGTDFTLKISRVEAE |
| | | DDGVYHCMQGTHWPLLAFGGGTKVEIGGGSSRSSSSGGGGSGGGGEVQLVQS |
| | | GAEVKKPGESLKISCKGSGYSFTSYWIGWVRQMPGKGLEWMGIIYPADSDTRY |
| | | SPSFQGQVTISADKSLNTAYLQWRSLKASDTAMYYCARIGDFYYYSGMDVWG |
| | | QGTTVTVSSASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDEQDHQEVSYA |
| 662 | scFv_84 | MALPVTALLLPLALLLHAARPGSDIVMTQTPLSSPVTLGQPASISCRSSQSLVHSD |
| | CAR | GNTFLSWLQQRPGQPPRLLIYKISNRFSGVPDRFSGSGAGTDFTLKISRVEAEDV |
| | | GVYYCMQATQFPLTFGGGTKVEIKGGGSSRSSSSGGGGSGGGGEVQLVQSGAE |
| | | VKKPGESLKISCKGSGYIFTSYWIAWVRQMPGKGLEWMGIIYPGDSDTRYSPSF |
| | | QGQITISADKSISTAYLQWSSLKASDTAMYYCARVGTTNYYYGMDVWGQGTSV |
| | | TVSSASEQKLISEEDLPSGGGGSGGGGSSPTEPSSKTGNPRHLHVLIGTSVVKIPF |
| | | TILLFFLLHRWCSNKKNAAVMDQEPAGNRTVNSEDSDEQDHQEVSYA |
| 663 | scFv_85 | MALPVTALLLPLALLLHAARPGSDIVMTQTPLSSPVTLGQPASISCRSSQSLVHSD |
| | CAR | GHTYLSWLQQRPGQPPRLLFYKISNRFSGVPDRFSGSGAGTDFTLKISRVEAEDV |
| | | GIYYCMQATQFPLTFGGGTKVDIKGGGSSRSSSSGGGGSGGGGEVQLVQSGAEV |
| | | KKPGESLKISCKGSGYSFTTYWIGWVRQMPGKGLEWMGIIYPGDSDTRYSPSFQ |
| | | GQVTISADKSISTAYLQWSSLKASDTAMYYCARIGDYYSYSGLDVWGQGTTVT |
| | | VSSASEQKLISEEDLPSGGGGSGGGGSSPTEPSSKTGNPRHLHVLIGTSVVKIPFTI |
| | | LLFFLLHRWCSNKKNAAVMDQEPAGNRTVNSEDSDEQDHQEVSYA |
| 664 | scFv_86 | MALPVTALLLPLALLLHAARPGSQPVLTQPTSLSASPGASARLTCTLRSGINLGS |
| | CAR | YRIFWYQQKPESPPRYLLSYYSDSRKHQGSGVPSRFSGSKDASSNAGILVISGLQ |
| | | SEDEADYYCIFWHSSAWVFGGGTKLTVGGGSSRSSSSGGGGSGGGGQVQLQQS |
| | | GPGLMKPSQTLSLTCAISGDSVSSNSVSWNWIRQSPSRGLEWLGRTYYRFKWFN |
| | | DYAVSVKSRITINPDTSKNQFSLRLNSVTPEDTALYYCARIDIWNDVFDYWGQGT |
| | | LVTVSSASEQKLISEEDLPSGGGGSGGGGSSPTEPSSKTGNPRHLHVLIGTSVVKI |
| | | PFTILLFFLLHRWCSNKKNAAVMDQEPAGNRTVNSEDSDEQDHQEVSYA |
| 809 | | MALPVTALLLPLALLLHAARPEVQLQQSGAELVKPGASVKMSCKASGYTFTSY |
| | | NMHWVKQTPGQGLEWIGAIYPGNGDTSYNQKFKGKATLTADKSSSTAYMQLSS |
| | | LTSEDSADYYCARSNYYGSSYWFFDVWGAGTTVTVSSGSTSGGGSGGGSGGG |
| | | GSSDIVLTQSPAILSASPGEKVTMTCRASSSVNYMDWYQKKPGSSPKPWIYATSN |
| | | LASGVPARFSGSGSGTSYSLTISRVEAEDAATYYCQQWSFNPPTFGGGTKLEIKPS |
| | | GGGGSGGGGSGGGGSGGGGSGGGGSPYEWSNSSDPLLVSVTGNPSNSWPSPTE |
| | | PSSKTGNPRHLHVLIGTSVVKIPFTILLFFLLHRWCSNKKNAAVMDQEPAGNRTV |
| | | NSEDSDEQDHQEVSYA |

The exemplary CAR of SEQ ID NOS: 620-644 comprise the amino acid sequence of SPTEPSSKTGNPRHLHVLIGTSVVKIPFTILLFFLLHRWCSNKKNAAVMDQEPAGNRTV NSEDSDEQDHQEVSYA (SEQ ID NO: 266). In some embodiments, the second fusion protein comprises the sequence of one of SEQ ID NOS: 620-644 with the sequence of SEQ ID NO: 266 replaced by PYEWSNSSDPLLVSVTGNPSNSWPSPTEPSSKTGNPRHLHVLIGTSVVKIPFTILLFFLLH RWCSNKKNAAVMDQEPAGNRTVNSEDSDEQDHQEVSYA (SEQ ID NO: 744). In some embodiments, the second fusion protein comprises a functional variant of one of the sequences of SEQ ID NOS: 620-644 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of the respective one of SEQ ID NOS: 620-644, where the sequence of SEQ ID NO: 266 is replaced by a functional variant of SEQ ID NO: 741 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of SEQ ID NO: 744.

In some embodiments, the CAR comprises an amino acid sequence having about 70%, about 75%, about 80%, about 85%, about 90%, about 91%, about 92%, about 93%, about 94%, about 95%, about 96%, about 97%, about 98%, about 99% identity to the amino acid sequence of SEQ ID NO: 809. In some embodiments, the CAR comprises the amino acid sequence of SEQ ID NO: 809.

In some embodiments, the first and/or second fusion protein comprises a DAP12 association sequence.

In some embodiments, the DAP12 association sequence comprises a basic amino acid residue. In some embodiments, the basic amino acid residue is an arginine or a lysine. In some embodiments, the amino acid residue is an arginine. In some embodiments the arginine is the arginine of SEQ ID NO: 753. In some embodiments, the association sequence comprises the amino acid sequence of SEQ ID NO: 753. In some embodiments, the association sequence comprises the amino acid sequence of a functional variant of the amino acid sequence of SEQ ID NOS: 753 having at least about 70%, about 75%, about 80%, about 85%, about 90%, about 91%, about 92%, about 93%, about 94%, about 95%, about 96%, about 97%, about 98%, about 99% or more sequence identity to the amino acid sequence of the respective one of SEQ ID NOS: 753.

### Third Nucleic Acid Sequence

As described above, in some embodiments, the composition of the disclosure further includes a third nucleic acid sequence encoding a DAP12 polypeptide.

In some embodiments, the third nucleic acid encoding a DAP12 polypeptide comprises the sequence of SEQ ID NO: 666, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 666. See Table 3. In some embodiments, the third nucleic acid encodes an amino acid sequence comprising the amino acid sequence of SEQ ID NO: 667, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 667. See Table 9.

In some embodiments, the DAP12 polypeptide comprises an extracellular domain, a transmembrane domain, and a cytoplasmic domain. In some embodiments, the DAP12 extracellular domain comprises the amino acid sequence of SEQ ID NO: 747, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 747. In some embodiments, the DAP12 transmembrane domain comprises the amino acid sequence of SEQ ID NO: 748, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 748. In some embodiments, the DAP12 cytoplasmic domain comprises the amino acid sequence of SEQ ID NO: 749, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 749.

In some embodiments, the transmembrane domain of a DAP12 polypeptide comprises an interaction sequence capable of associating with the transmembrane domain of the first fusion protein or the second fusion protein. In some embodiments, the DAP12 comprises an ITIM. In some embodiments, the DAP12 comprises an ITAM. In some embodiments, the interaction sequence comprises the amino acid sequence of one of SEQ ID NOS: 750. In some embodiments, the interaction sequence comprises a functional variant of the amino acid sequence of one of SEQ ID NOS: 750 having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 750. In some embodiments, the interaction sequence comprises one or more negatively charged amino acids residues capable of associating with one or more basic amino acid residues of the association sequence of a transmembrane domain. In some embodiments, the one or more negatively charged amino acid residues comprise an aspartate. In some embodiments, the aspartate is at position 10 of the transmembrane domain. In some embodiments, the aspartate is at position 10 of SEQ ID NO: 748. In some embodiments, the DAP12 comprises an ITAM having the sequence of SEQ ID NO: 751, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 751. In some embodiments, the DAP12 comprises an ITIM having the sequence of SEQ ID NO: 752, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 752.

In some embodiments, one or more KIR-CAR herein comprise an inhibitory KIR which delivers its signal through one or more immunotyrosine-based inhibitory motifs (ITIMs) that interact directly or indirectly with cytoplasmic signaling proteins such as SHP-1, SHP-2 and Vav family of proteins. KIRs bearing cytoplasmic domains that contain (ITIMs) abrogate the activating signal leading to inhibition of NK cytolytic and cytokine producing activity. In some embodiments, a modified T cell expressing a KIR-CAR herein can elicit a KIR-CAR-mediated T-cell response. In some embodiments, the dependence of the binding to more than one type of antigen allows the modified T cell to exhibit a heightened specificity to elicit a response upon binding of a tumor cell rather than a normal bystander cell.

TABLE 9
DAP12

| SEQ | | |
|---|---|---|
| ID NO: | Name: | Sequence: |
| 666 | DAP12 DNA | ATGGGCGGCCTGGAACCGTGCAGCCGC |
| | | TTTCTGCTGCTGCCGCTGCTGCTGGCG |
| | | GTGAGCGGCCTGCGCCCGGTGCAGGTG |
| | | CAGGCGCAGAGCGATTGCAGCTGCAGC |
| | | ACCGTGAGCCCGGGCGTGCTGGCGGGC |
| | | ATTGTGATGGGCGATCTGGTGCTGACV |
| | | GTGCTGATTGCGCTGGCGGTGTATTTT |
| | | CTGGGCCGCCTGGTGCCGCGCGGCCGC |
| | | GGCGCGGCGGAAGCGGCGACCCGCAAA |
| | | CAGCGCATTACCGAAACCGAAAGCCCG |
| | | TATCAGGAACTGCAGGGCCAGCGCAGC |
| | | GATGTGTATAGCGATCTGAACACCCAG |
| | | CGCCCGTATTATAAA |
| 667 | DAP12 | MGGLEPCSRFLLLPLLLAVSGLRPVQV |
| | | QAQSDCSCSTVSPGVLAGIVMGDLVLT |
| | | VLIALAVYFLGRLVPRGRGAAEAATRK |
| | | QRITETESPYQELQGQRSDVYSDLNTQ |
| | | RPYYK |
| 747 | DAP12 | MGGLEPCSRFLLLPLLLAVSGLRPVQV |
| | Extracellular | QAQSDCSCSTVSP |
| | Domain | |
| 748 | DAP12 | GVLAGIVMGDLVLTVLIALAV |
| | Transmembrane | |
| | Domain | |
| 749 | DAP12 | YFLGRLVPRGRGAAEAATRKQRITETES |
| | Cytoplasmic | PYQELQGQRSDVYSDLNTQRPYYK |
| | Domain | |
| 750 | DAP12 | MGDLVL |
| | Interaction | |
| | Sequence | |
| 751 | DAP12 ITAM | ESPYQELQGQRSDVYSDL |
| 752 | DAP12 ITIM | SPYQEL |

### Dual KIR-CARs

The present disclosure also encompasses dual KIR-CARs. A dual-KIR chimeric antigen receptor (CAR) is an engineered fusion protein that incorporates two independent activating KIR-based CAR units within a single genetic construct, enabling simultaneous or independent recognition of CD19 and CD20 on malignant B cells.

In some embodiments, a composition herein comprises a first nucleic acid molecule. In some embodiments, the first nucleic acid molecule comprises both the first nucleic acid sequence and the second nucleic acid. In some embodiments, the first nucleic acid sequence and the second nucleic acid sequence within the first nucleic acid molecule are non-contiguous. In some embodiments, the first nucleic acid sequence and the second nucleic acid sequence within the first nucleic acid molecule are non-contiguous and separated by a nucleic acid sequence encoding a cleavable linker sequence or a ribosome skipping peptide.

In exemplary embodiments, the dual-KIR CAR is expressed from a bicistronic or tricistronic nucleic acid, in which the anti-CD19 KIR-CAR and anti-CD20 KIR-CAR are encoded within a single open reading frame and separated by ribosomal skip sequences such as P2A, E2A, or T2A peptides.

In some embodiments, the cleavable linker sequence or ribosome skipping peptide comprises an amino acid sequence selected from P2A (ATNFSLLKQAGDVEENPGP, SEQ ID NO: 677), E2A (QCTNYALLKLAGDVESNPGP, SEQ ID NO: 678), T2A (EGRGSLLTCGDVEENPGP, SEQ ID NO: 679), F2A (VKQTLNFDLLKLAGDVESNPGP, SEQ ID NO: 680), and furin (PRRAR, SEQ ID NO: 808), or functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 677-680 or 808. In some embodiments, nucleic acid sequence encoding a ribosome skipping peptide encodes P2A (e.g., GCCACGAACTTCTCTCTGTTAAAGCAAGCAGGAGACGTGGAAGAAAACCCCGGTC CT, SEQ ID NO: 834), E2A (e.g., CAGTGCACCAACTACGCCCTGCTGAAGCTGGCCGGCGACGTGGAGAGCAACCCCG GCCCC, SEQ ID NO: 835), T2A (e.g., gagggcagaggaagtcttctaacatgcggtgacgtggaggagaatcccggccct, SEQ ID NO: 836), or F2A (e.g., GTGAAGCAGACCCTGAACTTCGACCTGCTGAAGCTGGCCGGCGACGTGGAGAGCA ACCCCGGCCCC, SEQ ID NO: 697). In some embodiments, the nucleic acid sequence encoding a cleavable linker sequence or ribosome skipping peptide is a functional variant of SEQ ID NO: 834, 835, 836, or 697 having at least about 70%, about 75%, about 80%, about 85%, about 90%, about 91%, about 92%, about 93%, about 94%, about 95%, about 96%, about 97%, about 98%, about 99% or more sequence identity SEQ ID NO: 834, 835, 836, or 697. In some embodiments, the non-contiguous first and second nucleic acid sequences are linked by an IRES site. In some embodiments, the non-contiguous first and second nucleic acid sequences are linked by a furin site. In some embodiments, the first nucleic acid molecule comprises, in the 5′ to 3′ orientation, a regulatory sequence the first nucleic acid sequence, a furin linker, and the second nucleic acid sequence. In some embodiments, the first nucleic acid molecule further comprises the third nucleic acid sequence. In some embodiments, the nucleic acid molecule is a DNA. In some embodiments, the nucleic acid sequence is an RNA. In some embodiments, the RNA is an mRNA.

In some embodiments, the composition comprises a vector. In some embodiments, the vector comprises the one or more of first nucleic acid sequence, the second nucleic acid sequence, and the third nucleic acid sequence. In some embodiments, the vector comprises the first nucleic acid molecule. In some embodiments, the vector is the first nucleic molecule. In some embodiments, the vector is an expression vector. In some embodiments, the expression vector comprises one or more regulatory sequences. In some embodiments, the one or more regulatory sequences are capable of causing expression of one or both of the first nucleic acid sequence and the second nucleic acid sequence. In some embodiments, the one or more regulatory sequences are capable of causing expression of the third nucleic acid sequence.

In some embodiments, the composition comprises a second nucleic acid molecule comprising the third nucleic acid sequence. In some embodiments, the second nucleic acid molecule is a second vector. In some embodiments, the second vector is an expression vector. In some embodiments, the second expression vector comprises one or more regulatory sequences. In some embodiments, the one or more regulatory sequences are capable of causing expression of the third nucleic acid sequence.

In some embodiments, one or both of the first fusion protein and the second fusion protein comprise a co-stimulatory domain. In some embodiments, the co-stimulatory domain is 4-1BB, OX40, CD2, CD27, or CD28. In some embodiments, the co-stimulatory domain comprises an amino acid sequence of one of SEQ ID NOS: 665, 773, 776, 779, or 783. See Table 10. In some embodiments, the co-stimulatory domain comprises a functional variant of an amino acid sequence of one of SEQ ID NOS: 665, 773, 776, 779, or 783 having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 665, 773, 776, 779, or 783. In some embodiments, the co-stimulatory domain comprises one or more amino acid sequence of SEQ ID NOS: 774 or 775, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 774 or 775. In some embodiments, the co-stimulatory domain comprises one or more amino acid sequence of SEQ ID NOS: 777 or 778, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 777 or 778. In some embodiments, the co-stimulatory domain comprises one or more amino acid sequence of SEQ ID NOS: 780 or 781, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 780 or 781. In some embodiments, the co-stimulatory domain comprises one or more amino acid sequence of SEQ ID NOS: 783, 784, or 785, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 783, 784, or 785.

TABLE 10
Co-Stimulatory Domains

| SEQ | | |
|---|---|---|
| ID NO: | Name: | Sequence: |
| 665 | 4-1BB | KRGRKKLLYIFKQPFMRPVQTTQEEDGCSCRFPEEEEGGCEL |
| 773 | OX40 | VAAILGLGLVLGLLGPLAILLALYLLRRDQRLPPDAHKPPGGGSFRTPIQEEQA |
| | | DAHSTLAKI |
| 774 | OX40 Trans- | VAAILGLGLVLGLLGPLAILL |
| | membrane | |
| | Domain | |
| 775 | OX40 | ALYLLRRDQRLPPDAHKPPGGGSFRTPIQEEQADAHSTLAKI |
| | Cytoplasmic | |
| | Domain | |
| 776 | CD2 | IYLIIGICGGGSLLMVFVALLVFYITKRKKQRSRRNDEELETRAHRVATEERG |
| | | RKPHQIPASTPQNPATSQHPPPPPGHRSQAPSHRPPPPGHRVQHQPQKRPPAP |
| | | SGTQVHQQKGPPLPRPRVQPKPPHGAAENSLSPSSN |
| 777 | CD2 Trans- | IYLIIGICGGGSLLMVFVALLVFYIT |
| | membrane | |
| | Domain | |
| 778 | CD2 | KRKKQRSRRNDEELETRAHRVATEERGRKPHQIPASTPQNPATSQHPPPPPG |
| | Cytoplasmic | HRSQAPSHRPPPPGHRVQHQPQKRPPAPSGTQVHQQKGPPLPRPRVQPKPPH |
| | Domain | GAAENSLSPSSN |
| 779 | CD27 | ILVIFSGMFLVFTLAGALFLHQRRKYRSNKGESPVEPAEPCHYSCPREEEGSTI |
| | | PIQEDYRKPEPACSP |
| 780 | CD27 Trans- | ILVIFSGMFLVFTLAGALFLH |
| | membrane | |
| | Domain | |
| 781 | CD27 | QRRKYRSNKGESPVEPAEPCHYSCPREEEGSTIPIQEDYRKPEPACSP |
| | Cytoplasmic | |
| | Domain | |
| 782 | CD28 | IEVMYPPPYLDNEKSNGTIIHVKGKHLCPSPLFPGPSKPFWVLVVVGGVLAC |
| | | YSLLVTVAFIIFWVRSKRSRLLHSDYMNMTPRRPGPTRKHYQPYAPPRDFAA |
| | | YRS |
| 783 | CD28 Hinge | IEVMYPPPYLDNEKSNGTIIHVKGKHLCPSPLFPGPSKP |
| | (extracellular) | |
| 784 | CD28 Trans- | FWVLVVVGGVLACYSLLVTVAFIIFWV |
| | membrane | |
| | Domain | |
| 785 | CD28 | RSKRSRLLHSDYMNMTPRRPGPTRKHYQPYAPPRDFAAYRS |
| | Cytoplasmic | |
| | Domain | |

In some embodiments, the nucleic acid construct encoding the dual KIR described herein further comprises a sequence encoding DAP12. In some embodiments, the DAP12 sequence comprises SEQ ID NO: 667 or a functional variant thereof preceding a cleavage/ribosome skipping site. In some embodiments, the cleavage site is P2A (ATNFSLLKQAGDVEENPGP, SEQ ID NO: 677), E2A (QCTNYALLKLAGDVESNPGP, SEQ ID NO: 678), T2A (EGRGSLLTCGDVEENPGP, SEQ ID NO: 679), F2A (VKQTLNFDLLKLAGDVESNPGP, SEQ ID NO: 680), or furin (PRRAR, SEQ ID NO: 808), or functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 677-680 or 808. In some embodiments of a fusion protein herein, about 1, 2, 3, 4, 5, 6, 7, 8, 9, or 10 amino acids separate DAP12 or a functional variant thereof and a cleavage/ribosome skipping site. In some embodiments, the amino acids separating DAP12 or a functional variant thereof and a cleavage/ribosome skipping site comprise VEGGG (SEQ ID NO: 745). In some embodiments of a fusion protein herein, a DAP12 or a functional variant thereof precedes an anti-CD19 binder or an anti-CD20 binder. In some embodiments, a nucleic acid sequence encoding a fusion protein herein comprises an IRES site after a sequence encoding DAP12 or a functional variant and before a sequence encoding an anti CD19 or CD20 binder. In some embodiments of a fusion protein herein, a DAP12 or a functional variant thereof and a cleavage site precedes an anti-CD19 binder or an anti-CD20 binder. In some embodiments of a fusion protein herein, an arginine is inserted before one or both of an anti-CD19 binder or an anti-CD20 binder.

### Exemplary Dual KIR Constructs

Exemplary dual KIR fusion proteins of embodiments herein are set forth in Table 11. Some embodiments comprise a nucleic acid sequence encoding one of SEQ ID NOS: 681-688, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 681-688. Some embodiments comprise a nucleic acid sequence comprising one of SEQ ID NOS: 689-696 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 689-696.

TABLE 11
Exemplary Fusion Proteins

| SEQ ID. NO. | Name | Sequence |
|---|---|---|
| 681 | 1-T19_P20 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGEGRGSLLTCGDVEENPGPRMALPV |
| | | TALLLPLALLLHAARPGSQSALTQTASVSGSLGQKVTISCTGSRSNIGA |
| | | NYVGWYQQVPGIGPRTVIFGYNYRPSGVPDRFSSSKSGNSATLTISGL |
| | | QAEDEAEYYCSTWDSSLNAYVFGSGTQLTVLGGGSSRSSSSGGGGSG |
| | | GGGEVPLVESGGDLVKPGGSLRLSCVASGFTLSNYGMSWVRQSPGKG |
| | | LQWVAAIRSGGNTYYADAVKGRFTISRDNAKNTLYLQMNSLRAEDTA |
| | | VYYCASQWGDWGHFEYWGQGTLVTVSSASEQKLISEEDLPSGGGGS |
| | | GGGGSSPTEPSSKTGNPRHLHVLIGTSVVKIPFTILLFFLLHRWCSNKK |
| | | NAAVMDQEPAGNRTVNSEDSDEQDHQEVSYAVEGGGATNFSLLKQA |
| | | GDVEENPGPRMALPVTALLLPLALLLHAARPEVQLQQSGAELVKPGA |
| | | SVKMSCKASGYTFTSYNMHWVKQTPGQGLEWIGAIYPGNGDTSYNQ |
| | | KFKGKATLTADKSSSTAYMQLSSLTSEDSADYYCARSNYYGSSYWFF |
| | | DVWGAGTTVTVSSGSTSGGGSGGGSGGGGSSDIVLTQSPAILSASPGE |
| | | KVTMTCRASSSVNYMDWYQKKPGSSPKPWIYATSNLASGVPARFSGS |
| | | GSGTSYSLTISRVEAEDAATYYCQQWSFNPPTFGGGTKLEIKGASHHH |
| | | HHHPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 682 | 2-T20_P19 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGEGRGSLLTCGDVEENPGPRMALPV |
| | | TALLLPLALLLHAARPEVQLQQSGAELVKPGASVKMSCKASGYTFTS |
| | | YNMHWVKQTPGQGLEWIGAIYPGNGDTSYNQKFKGKATLTADKSSS |
| | | TAYMQLSSLTSEDSADYYCARSNYYGSSYWFFDVWGAGTTVTVSSG |
| | | STSGGGSGGGSGGGGSSDIVLTQSPAILSASPGEKVTMTCRASSSVNY |
| | | MDWYQKKPGSSPKPWIYATSNLASGVPARFSGSGSGTSYSLTISRVEA |
| | | EDAATYYCQQWSFNPPTFGGGTKLEIKGASHHHHHHPSGGGGSGGG |
| | | GSSPTEPSSKTGNPRHLHVLIGTSVVKIPFTILLFFLLHRWCSNKKNAA |
| | | VMDQEPAGNRTVNSEDSDEQDHQEVSYAVEGGGATNFSLLKQAGDV |
| | | EENPGPRMALPVTALLLPLALLLHAARPGSQSALTQTASVSGSLGQKV |
| | | TISCTGSRSNIGANYVGWYQQVPGIGPRTVIFGYNYRPSGVPDRFSSSK |
| | | SGNSATLTISGLQAEDEAEYYCSTWDSSLNAYVFGSGTQLTVLGGGSS |
| | | RSSSSGGGGSGGGGEVPLVESGGDLVKPGGSLRLSCVASGFTLSNYG |
| | | MSWVRQSPGKGLQWVAAIRSGGNTYYADAVKGRFTISRDNAKNTLY |
| | | LQMNSLRAEDTAVYYCASQWGDWGHFEYWGQGTLVTVSSASEQKLI |
| | | SEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 683 | 3-P20_P19 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGATNFSLLKQAGDVEENPGPRMALP |
| | | VTALLLPLALLLHAARPEVQLQQSGAELVKPGASVKMSCKASGYTFT |
| | | SYNMHWVKQTPGQGLEWIGAIYPGNGDTSYNQKFKGKATLTADKSS |
| | | STAYMQLSSLTSEDSADYYCARSNYYGSSYWFFDVWGAGTTVTVSS |
| | | GSTSGGGSGGGSGGGGSSDIVLTQSPAILSASPGEKVTMTCRASSSVN |
| | | YMDWYQKKPGSSPKPWIYATSNLASGVPARFSGSGSGTSYSLTISRVE |
| | | AEDAATYYCQQWSFNPPTFGGGTKLEIKGASHHHHHHPSGGGGSGG |
| | | GGSSPTEPSSKTGNPRHLHVLIGTSVVKIPFTILLFFLLHRWCSNKKNA |
| | | AVMDQEPAGNRTVNSEDSDEQDHQEVSYAVEGGGATNFSLLKQAGD |
| | | VEENPGPRMALPVTALLLPLALLLHAARPGSQSALTQTASVSGSLGQK |
| | | VTISCTGSRSNIGANYVGWYQQVPGIGPRTVIFGYNYRPSGVPDRFSSS |
| | | KSGNSATLTISGLQAEDEAEYYCSTWDSSLNAYVFGSGTQLTVLGGGS |
| | | SRSSSSGGGGSGGGGEVPLVESGGDLVKPGGSLRLSCVASGFTLSNYG |
| | | MSWVRQSPGKGLQWVAAIRSGGNTYYADAVKGRFTISRDNAKNTLY |
| | | LQMNSLRAEDTAVYYCASQWGDWGHFEYWGQGTLVTVSSASEQKLI |
| | | SEEDLPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 684 | 4-P19_P20 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGATNFSLLKQAGDVEENPGPRMALP |
| | | VTALLLPLALLLHAARPGSQSALTQTASVSGSLGQKVTISCTGSRSNIG |
| | | ANYVGWYQQVPGIGPRTVIFGYNYRPSGVPDRFSSSKSGNSATLTISG |
| | | LQAEDEAEYYCSTWDSSLNAYVFGSGTQLTVLGGGSSRSSSSGGGGS |
| | | GGGGEVPLVESGGDLVKPGGSLRLSCVASGFTLSNYGMSWVRQSPGK |
| | | GLQWVAAIRSGGNTYYADAVKGRFTISRDNAKNTLYLQMNSLRAEDT |
| | | AVYYCASQWGDWGHFEYWGQGTLVTVSSASEQKLISEEDLPSGGGG |
| | | SGGGGSSPTEPSSKTGNPRHLHVLIGTSVVKIPFTILLFFLLHRWCSNK |
| | | KNAAVMDQEPAGNRTVNSEDSDEQDHQEVSYAVEGGGATNFSLLKQ |
| | | AGDVEENPGPRMALPVTALLLPLALLLHAARPEVQLQQSGAELVKPG |
| | | ASVKMSCKASGYTFTSYNMHWVKQTPGQGLEWIGAIYPGNGDTSYN |
| | | QKFKGKATLTADKSSSTAYMQLSSLTSEDSADYYCARSNYYGSSYWF |
| | | FDVWGAGTTVTVSSGSTSGGGSGGGSGGGGSSDIVLTQSPAILSASPG |
| | | EKVTMTCRASSSVNYMDWYQKKPGSSPKPWIYATSNLASGVPARFSG |
| | | SGSGTSYSLTISRVEAEDAATYYCQQWSFNPPTFGGGTKLEIKGASHH |
| | | HHHHPSGGGGSGGGGSSPTEPSSKTGNPRHLHVLIGTSVVKIPFTILLF |
| | | FLLHRWCSNKKNAAVMDQEPAGNRTVNSEDSDEQDHQEVSYA |
| 685 | 5-T20_T19 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGEGRGSLLTCGDVEENPGPRMALPV |
| | | TALLLPLALLLHAARPEVQLQQSGAELVKPGASVKMSCKASGYTFTS |
| | | YNMHWVKQTPGQGLEWIGAIYPGNGDTSYNQKFKGKATLTADKSSS |
| | | TAYMQLSSLTSEDSADYYCARSNYYGSSYWFFDVWGAGTTVTVSSG |
| | | STSGGGSGGGSGGGGSSDIVLTQSPAILSASPGEKVTMTCRASSSVNY |
| | | MDWYQKKPGSSPKPWIYATSNLASGVPARFSGSGSGTSYSLTISRVEA |
| | | EDAATYYCQQWSFNPPTFGGGTKLEIKGASHHHHHHPSGGGGSGGG |
| | | GSSPTEPSSKTGNPRHLHVLIGTSVVKIPFTILLFFLLHRWCSNKKNAA |
| | | VMDQEPAGNRTVNSEDSDEQDHQEVSYAVEGGGEGRGSLLTCGDVEE |
| | | NPGPRMALPVTALLLPLALLLHAARPGSQSALTQTASVSGSLGQKVTI |
| | | SCTGSRSNIGANYVGWYQQVPGIGPRTVIFGYNYRPSGVPDRFSSSKS |
| | | GNSATLTISGLQAEDEAEYYCSTWDSSLNAYVFGSGTQLTVLGGGSSR |
| | | SSSSGGGGSGGGGEVPLVESGGDLVKPGGSLRLSCVASGFTLSNYGMS |
| | | WVRQSPGKGLQWVAAIRSGGNTYYADAVKGRFTISRDNAKNTLYLQ |
| | | MNSLRAEDTAVYYCASQWGDWGHFEYWGQGTLVTVSSASEQKLISE |
| | | EDLPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 686 | 6-T19_T20 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGEGRGSLLTCGDVEENPGPRMALPV |
| | | TALLLPLALLLHAARPGSQSALTQTASVSGSLGQKVTISCTGSRSNIGA |
| | | NYVGWYQQVPGIGPRTVIFGYNYRPSGVPDRESSSKSGNSATLTISGL |
| | | QAEDEAEYYCSTWDSSLNAYVFGSGTQLTVLGGGSSRSSSSGGGGSG |
| | | GGGEVPLVESGGDLVKPGGSLRLSCVASGFTLSNYGMSWVRQSPGKG |
| | | LQWVAAIRSGGNTYYADAVKGRFTISRDNAKNTLYLQMNSLRAEDTA |
| | | VYYCASQWGDWGHFEYWGQGTLVTVSSASEQKLISEEDLPSGGGGS |
| | | GGGGSSPTEPSSKTGNPRHLHVLIGTSVVKIPFTILLFFLLHRWCSNKK |
| | | NAAVMDQEPAGNRTVNSEDSDEQDHQEVSYAVEGGGEGRGSLLTCG |
| | | DVEENPGPRMALPVTALLLPLALLLHAARPEVQLQQSGAELVKPGAS |
| | | VKMSCKASGYTFTSYNMHWVKQTPGQGLEWIGAIYPGNGDTSYNQ |
| | | KFKGKATLTADKSSSTAYMQLSSLTSEDSADYYCARSNYYGSSYWFF |
| | | DVWGAGTTVTVSSGSTSGGGSGGGSGGGGSSDIVLTQSPAILSASPGE |
| | | KVTMTCRASSSVNYMDWYQKKPGSSPKPWIYATSNLASGVPARFSGS |
| | | GSGTSYSLTISRVEAEDAATYYCQQWSFNPPTFGGGTKLEIKGASHHH |
| | | HHHPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 687 | 7-P20_T19 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGATNFSLLKQAGDVEENPGPRMALP |
| | | VTALLLPLALLLHAARPEVQLQQSGAELVKPGASVKMSCKASGYTFT |
| | | SYNMHWVKQTPGQGLEWIGAIYPGNGDTSYNQKFKGKATLTADKSS |
| | | STAYMQLSSLTSEDSADYYCARSNYYGSSYWFFDVWGAGTTVTVSS |
| | | GSTSGGGSGGGSGGGGSSDIVLTQSPAILSASPGEKVTMTCRASSSVN |
| | | YMDWYQKKPGSSPKPWIYATSNLASGVPARFSGSGSGTSYSLTISRVE |
| | | AEDAATYYCQQWSFNPPTFGGGTKLEIKGASHHHHHHPSGGGGSGG |
| | | GGSSPTEPSSKTGNPRHLHVLIGTSVVKIPFTILLFFLLHRWCSNKKNA |
| | | AVMDQEPAGNRTVNSEDSDEQDHQEVSYAVEGGGEGRGSLLTCGDVE |
| | | ENPGPRMALPVTALLLPLALLLHAARPGSQSALTQTASVSGSLGQKVT |
| | | ISCTGSRSNIGANYVGWYQQVPGIGPRTVIFGYNYRPSGVPDRFSSSKS |
| | | GNSATLTISGLQAEDEAEYYCSTWDSSLNAYVFGSGTQLTVLGGGSSR |
| | | SSSSGGGGSGGGGEVPLVESGGDLVKPGGSLRLSCVASGFTLSNYGMS |
| | | WVRQSPGKGLQWVAAIRSGGNTYYADAVKGRFTISRDNAKNTLYLQ |
| | | MNSLRAEDTAVYYCASQWGDWGHFEYWGQGTLVTVSSASEQKLISE |
| | | EDLPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 688 | 8-P19_T20 | MGGLEPCSRFLLLPLLLAVSGLRPVQVQAQSDCSCSTVSPGVLAGIVM |
| | AA | GDLVLTVLIALAVYFLGRLVPRGRGAAEAATRKQRITETESPYQELQG |
| | | QRSDVYSDLNTQRPYYKVEGGGATNFSLLKQAGDVEENPGPRMALP |
| | | VTALLLPLALLLHAARPGSQSALTQTASVSGSLGQKVTISCTGSRSNIG |
| | | ANYVGWYQQVPGIGPRTVIFGYNYRPSGVPDRESSSKSGNSATLTISG |
| | | LQAEDEAEYYCSTWDSSLNAYVFGSGTQLTVLGGGSSRSSSSGGGGS |
| | | GGGGEVPLVESGGDLVKPGGSLRLSCVASGFTLSNYGMSWVRQSPGK |
| | | GLQWVAAIRSGGNTYYADAVKGRFTISRDNAKNTLYLQMNSLRAEDT |
| | | AVYYCASQWGDWGHFEYWGQGTLVTVSSASEQKLISEEDLPSGGGG |
| | | SGGGGSSPTEPSSKTGNPRHLHVLIGTSVVKIPFTILLFFLLHRWCSNK |
| | | KNAAVMDQEPAGNRTVNSEDSDEQDHQEVSYAVEGGGEGRGSLLTC |
| | | GDVEENPGPRMALPVTALLLPLALLLHAARPEVQLQQSGAELVKPGA |
| | | SVKMSCKASGYTFTSYNMHWVKQTPGQGLEWIGAIYPGNGDTSYNQ |
| | | KFKGKATLTADKSSSTAYMQLSSLTSEDSADYYCARSNYYGSSYWFF |
| | | DVWGAGTTVTVSSGSTSGGGSGGGSGGGGSSDIVLTQSPAILSASPGE |
| | | KVTMTCRASSSVNYMDWYQKKPGSSPKPWIYATSNLASGVPARFSGS |
| | | GSGTSYSLTISRVEAEDAATYYCQQWSFNPPTFGGGTKLEIKGASHHH |
| | | HHHPSGGGGSGGGGSSPTEPSSKTGNPRHLHVLIGTSVVKIPFTILLFFL |
| | | LHRWCSNKKNAAVMDQEPAGNRTVNSEDSDEQDHQEVSYA |
| 689 | 1-T19_P20 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGAGGGCAGAG |
| | | GAAGTCTTCTAACATGCGGTGACGTGGAGGAGAATCCCGGCCCTAG |
| | | GATGGCCTTACCAGTGACCGCCTTGCTCCTGCCGCTGGCCTTGCTG |
| | | CTCCACGCCGCCAGGCCGGGATCCCAGTCTGCGCTGACTCAAACG |
| | | GCCTCAGTGTCTGGGTCCCTGGGCCAGAAGGTCACCATCTCCTGCA |
| | | CTGGAAGCAGGTCCAACATCGGTGCAAATTATGTGGGCTGGTATCA |
| | | ACAGGTCCCAGGAATAGGCCCTAGAACCGTCATTTTTGGTTATAATT |
| | | ATCGACCTTCAGGGGTCCCCGATCGATTCTCTAGCTCCAAGTCAGG |
| | | CAATTCAGCCACCCTGACCATCTCTGGGCTCCAGGCTGAGGACGAG |
| | | GCTGAGTATTATTGTTCAACATGGGACAGCAGTCTCAACGCTTACGT |
| | | GTTCGGCTCAGGGACCCAGCTGACCGTCCTCGGCGGTGGTTCCTCT |
| | | AGATCTTCCTCCTCTGGTGGCGGTGGCTCGGGCGGTGGTGGGGAG |
| | | GTGCCACTGGTGGAATCTGGGGGAGACCTGGTGAAGCCTGGGGGG |
| | | TCCTTGAGACTGTCCTGTGTGGCCTCTGGATTCACCCTCAGTAACTA |
| | | TGGCATGAGCTGGGTCCGTCAGTCTCCAGGGAAGGGGCTGCAGTG |
| | | GGTCGCAGCTATTAGGAGTGGTGGAAACACATACTACGCAGACGCT |
| | | GTGAAGGGCCGATTCACCATCTCCAGAGACAACGCCAAGAACACG |
| | | CTGTATCTGCAGATGAACAGCCTGAGAGCCGAGGACACGGCCGTG |
| | | TATTACTGTGCGTCCCAATGGGGTGATTGGGGCCACTTTGAGTACTG |
| | | GGGCCAGGGCACCCTGGTCACCGTCTCCTCAGCTAGCGAGCAGAA |
| | | ATTAATCTCAGAGGAGGACCTGCCTAGCGGTGGCGGAGGTTCTGGA |
| | | GGTGGGGGTTCCTCACCCACTGAACCAAGCTCCAAAACCGGTAAC |
| | | CCCAGACACCTGCATGTTCTGATTGGGACCTCAGTGGTCAAAATCC |
| | | CTTTCACCATCCTCCTCTTCTTTCTCCTTCATCGCTGGTGCTCCAAC |
| | | AAAAAAAATGCTGCTGTAATGGACCAAGAGCCTGCAGGGAACAGA |
| | | ACAGTGAACAGCGAGGATTCTGATGAACAAGACCATCAGGAGGTG |
| | | TCATACGCAGTCGAGGGCGGCGGAGCCACGAACTTCTCTCTGTTAA |
| | | AGCAAGCAGGAGACGTGGAAGAAAACCCCGGTCCTAGGATGGCCT |
| | | TACCAGTGACCGCCTTGCTCCTGCCGCTGGCCTTGCTGCTCCACGC |
| | | CGCCAGGCCGGAGGTGCAGCTGCAGCAGTCTGGGGCTGAGCTGGT |
| | | GAAGCCTGGGGCCTCAGTGAAGATGTCCTGCAAGGCTTCTGGCTAC |
| | | ACATTTACCAGTTACAATATGCACTGGGTAAAGCAGACACCTGGAC |
| | | AGGGCCTGGAATGGATTGGAGCTATTTATCCAGGAAATGGTGATACT |
| | | TCCTACAATCAGAAGTTCAAAGGCAAGGCCACATTGACTGCAGAC |
| | | AAATCCTCCAGCACAGCCTACATGCAGCTCAGCAGCCTGACATCTG |
| | | AGGACTCTGCGGACTATTACTGTGCAAGATCTAATTATTACGGTAGT |
| | | AGCTACTGGTTCTTCGATGTCTGGGGCGCAGGGACCACGGTCACCG |
| | | TCTCCTCAGGCAGTACTAGCGGTGGTGGCTCCGGGGGCGGTTCCGG |
| | | TGGGGGCGGCAGCAGCGACATTGTGCTGACCCAATCTCCAGCTATC |
| | | CTGTCTGCATCTCCAGGGGAGAAGGTCACAATGACTTGCAGGGCC |
| | | AGCTCAAGTGTAAATTACATGGACTGGTACCAGAAGAAGCCAGGTT |
| | | CCTCCCCCAAACCCTGGATTTATGCCACATCCAACCTGGCTTCTGGA |
| | | GTCCCTGCTCGCTTCAGTGGCAGTGGGTCTGGGACCTCTTACTCTC |
| | | TCACAATCAGCAGAGTGGAGGCTGAAGATGCTGCCACTTATTACTG |
| | | CCAGCAGTGGAGTTTTAATCCACCCACGTTCGGAGGGGGGACCAA |
| | | GCTGGAAATAAAAGGAGCTAGCCACCATCACCATCACCATCCTAGC |
| | | GGTGGCGGAGGTTCTGGAGGTGGGGGTTCCTCACCCACTGAACCA |
| | | AGCTCCAAAACCGGTAACCCCAGACACCTGCATGTTCTGATTGGGA |
| | | CCTCAGTGGTCAAAATCCCTTTCACCATCCTCCTCTTCTTTCTCCTT |
| | | CATCGCTGGTGCTCCAACAAAAAAAATGCTGCTGTAATGGACCAAG |
| | | AGCCTGCAGGGAACAGAACAGTGAACAGCGAGGATTCTGATGAAC |
| | | AAGACCATCAGGAGGTGTCATACGCATAATGAGC |
| 690 | 2-T20_P19 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGAGGGCAGAG |
| | | GAAGTCTTCTAACATGCGGTGACGTGGAGGAGAATCCCGGCCCTAG |
| | | GATGGCCTTACCAGTGACCGCCTTGCTCCTGCCGCTGGCCTTGCTG |
| | | CTCCACGCCGCCAGGCCGGAGGTGCAGCTGCAGCAGTCTGGGGCT |
| | | GAGCTGGTGAAGCCTGGGGCCTCAGTGAAGATGTCCTGCAAGGCT |
| | | TCTGGCTACACATTTACCAGTTACAATATGCACTGGGTAAAGCAGAC |
| | | ACCTGGACAGGGCCTGGAATGGATTGGAGCTATTTATCCAGGAAAT |
| | | GGTGATACTTCCTACAATCAGAAGTTCAAAGGCAAGGCCACATTGA |
| | | CTGCAGACAAATCCTCCAGCACAGCCTACATGCAGCTCAGCAGCCT |
| | | GACATCTGAGGACTCTGCGGACTATTACTGTGCAAGATCTAATTATT |
| | | ACGGTAGTAGCTACTGGTTCTTCGATGTCTGGGGCGCAGGGACCAC |
| | | GGTCACCGTCTCCTCAGGCAGTACTAGCGGTGGTGGCTCCGGGGGC |
| | | GGTTCCGGTGGGGGCGGCAGCAGCGACATTGTGCTGACCCAATCTC |
| | | CAGCTATCCTGTCTGCATCTCCAGGGGAGAAGGTCACAATGACTTG |
| | | CAGGGCCAGCTCAAGTGTAAATTACATGGACTGGTACCAGAAGAA |
| | | GCCAGGTTCCTCCCCCAAACCCTGGATTTATGCCACATCCAACCTG |
| | | GCTTCTGGAGTCCCTGCTCGCTTCAGTGGCAGTGGGTCTGGGACCT |
| | | CTTACTCTCTCACAATCAGCAGAGTGGAGGCTGAAGATGCTGCCAC |
| | | TTATTACTGCCAGCAGTGGAGTTTTAATCCACCCACGTTCGGAGGG |
| | | GGGACCAAGCTGGAAATAAAAGGAGCTAGCCACCATCACCATCAC |
| | | CATCCTAGCGGTGGCGGAGGTTCTGGAGGTGGGGGTTCCTCACCCA |
| | | CTGAACCAAGCTCCAAAACCGGTAACCCCAGACACCTGCATGTTCT |
| | | GATTGGGACCTCAGTGGTCAAAATCCCTTTCACCATCCTCCTCTTCT |
| | | TTCTCCTTCATCGCTGGTGCTCCAACAAAAAAAATGCTGCTGTAAT |
| | | GGACCAAGAGCCTGCAGGGAACAGAACAGTGAACAGCGAGGATT |
| | | CTGATGAACAAGACCATCAGGAGGTGTCATACGCAGTCGAGGGCG |
| | | GCGGAGCCACGAACTTCTCTCTGTTAAAGCAAGCAGGAGACGTGG |
| | | AAGAAAACCCCGGTCCTAGGATGGCCTTACCAGTGACCGCCTTGCT |
| | | CCTGCCGCTGGCCTTGCTGCTCCACGCCGCCAGGCCGGGATCCCAG |
| | | TCTGCGCTGACTCAAACGGCCTCAGTGTCTGGGTCCCTGGGCCAGA |
| | | AGGTCACCATCTCCTGCACTGGAAGCAGGTCCAACATCGGTGCAA |
| | | ATTATGTGGGCTGGTATCAACAGGTCCCAGGAATAGGCCCTAGAAC |
| | | CGTCATTTTTGGTTATAATTATCGACCTTCAGGGGTCCCCGATCGATT |
| | | CTCTAGCTCCAAGTCAGGCAATTCAGCCACCCTGACCATCTCTGGG |
| | | CTCCAGGCTGAGGACGAGGCTGAGTATTATTGTTCAACATGGGACA |
| | | GCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCT |
| | | CGGGCGGTGGTGGGGAGGTGCCACTGGTGGAATCTGGGGGAGACC |
| | | TGGTGAAGCCTGGGGGGTCCTTGAGACTGTCCTGTGTGGCCTCTGG |
| | | ATTCACCCTCAGTAACTATGGCATGAGCTGGGTCCGTCAGTCTCCA |
| | | GGGAAGGGGCTGCAGTGGGTCGCAGCTATTAGGAGTGGTGGAAAC |
| | | ACATACTACGCAGACGCTGTGAAGGGCCGATTCACCATCTCCAGAG |
| | | ACAACGCCAAGAACACGCTGTATCTGCAGATGAACAGCCTGAGAG |
| | | CCGAGGACACGGCCGTGTATTACTGTGCGTCCCAATGGGGTGATTG |
| | | GGGCCACTTTGAGTACTGGGGCCAGGGCACCCTGGTCACCGTCTCC |
| | | TCAGCTAGCGAGCAGAAATTAATCTCAGAGGAGGACCTGCCTAGCG |
| | | GTGGCGGAGGTTCTGGAGGTGGGGGTTCCTCACCCACTGAACCAA |
| | | GCTCCAAAACCGGTAACCCCAGACACCTGCATGTTCTGATTGGGAC |
| | | CTCAGTGGTCAAAATCCCTTTCACCATCCTCCTCTTCTTTCTCCTTC |
| | | ATCGCTGGTGCTCCAACAAAAAAAATGCTGCTGTAATGGACCAAGA |
| | | GCCTGCAGGGAACAGAACAGTGAACAGCGAGGATTCTGATGAACA |
| | | AGACCATCAGGAGGTGTCATACGCATAATGAGC |
| 691 | 3-P20_P19 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGCCACGAACTT |
| | | CTCTCTGTTAAAGCAAGCAGGAGACGTGGAAGAAAACCCCGGTCC |
| | | TAGGATGGCCTTACCAGTGACCGCCTTGCTCCTGCCGCTGGCCTTG |
| | | CTGCTCCACGCCGCCAGGCCGGAGGTGCAGCTGCAGCAGTCTGGG |
| | | GCTGAGCTGGTGAAGCCTGGGGCCTCAGTGAAGATGTCCTGCAAG |
| | | GCTTCTGGCTACACATTTACCAGTTACAATATGCACTGGGTAAAGCA |
| | | GACACCTGGACAGGGCCTGGAATGGATTGGAGCTATTTATCCAGGA |
| | | AATGGTGATACTTCCTACAATCAGAAGTTCAAAGGCAAGGCCACAT |
| | | TGACTGCAGACAAATCCTCCAGCACAGCCTACATGCAGCTCAGCAG |
| | | CCTGACATCTGAGGACTCTGCGGACTATTACTGTGCAAGATCTAATT |
| | | ATTACGGTAGTAGCTACTGGTTCTTCGATGTCTGGGGCGCAGGGAC |
| | | CACGGTCACCGTCTCCTCAGGCAGTACTAGCGGTGGTGGCTCCGGG |
| | | GGCGGTTCCGGTGGGGGCGGCAGCAGCGACATTGTGCTGACCCAA |
| | | TCTCCAGCTATCCTGTCTGCATCTCCAGGGGAGAAGGTCACAATGA |
| | | CTTGCAGGGCCAGCTCAAGTGTAAATTACATGGACTGGTACCAGAA |
| | | GAAGCCAGGTTCCTCCCCCAAACCCTGGATTTATGCCACATCCAAC |
| | | CTGGCTTCTGGAGTCCCTGCTCGCTTCAGTGGCAGTGGGTCTGGGA |
| | | CCTCTTACTCTCTCACAATCAGCAGAGTGGAGGCTGAAGATGCTGC |
| | | CACTTATTACTGCCAGCAGTGGAGTTTTAATCCACCCACGTTCGGA |
| | | GGGGGGACCAAGCTGGAAATAAAAGGAGCTAGCCACCATCACCAT |
| | | CACCATCCTAGCGGTGGCGGAGGTTCTGGAGGTGGGGGTTCCTCAC |
| | | CCACTGAACCAAGCTCCAAAACCGGTAACCCCAGACACCTGCATG |
| | | TTCTGATTGGGACCTCAGTGGTCAAAATCCCTTTCACCATCCTCCTC |
| | | TTCTTTCTCCTTCATCGCTGGTGCTCCAACAAAAAAAATGCTGCTGT |
| | | AATGGACCAAGAGCCTGCAGGGAACAGAACAGTGAACAGCGAGG |
| | | ATTCTGATGAACAAGACCATCAGGAGGTGTCATACGCAGTCGAGGG |
| | | CGGCGGAGCCACGAACTTCTCTCTGTTAAAGCAAGCAGGAGACGT |
| | | GGAAGAAAACCCCGGTCCTAGGATGGCCTTACCAGTGACCGCCTTG |
| | | CTCCTGCCGCTGGCCTTGCTGCTCCACGCCGCCAGGCCGGGATCCC |
| | | AGTCTGCGCTGACTCAAACGGCCTCAGTGTCTGGGTCCCTGGGCCA |
| | | GAAGGTCACCATCTCCTGCACTGGAAGCAGGTCCAACATCGGTGC |
| | | AAATTATGTGGGCTGGTATCAACAGGTCCCAGGAATAGGCCCTAGA |
| | | ACCGTCATTTTTGGTTATAATTATCGACCTTCAGGGGTCCCCGATCG |
| | | ATTCTCTAGCTCCAAGTCAGGCAATTCAGCCACCCTGACCATCTCTG |
| | | GGCTCCAGGCTGAGGACGAGGCTGAGTATTATTGTTCAACATGGGA |
| | | CAGCAGTCTCAACGCTTACGTGTTCGGCTCAGGGACCCAGCTGACC |
| | | GTCCTCGGCGGTGGTTCCTCTAGATCTTCCTCCTCTGGTGGCGGTG |
| | | GCTCGGGCGGTGGTGGGGAGGTGCCACTGGTGGAATCTGGGGGAG |
| | | ACCTGGTGAAGCCTGGGGGGTCCTTGAGACTGTCCTGTGTGGCCTC |
| | | TGGATTCACCCTCAGTAACTATGGCATGAGCTGGGTCCGTCAGTCTC |
| | | CAGGGAAGGGGCTGCAGTGGGTCGCAGCTATTAGGAGTGGTGGAA |
| | | ACACATACTACGCAGACGCTGTGAAGGGCCGATTCACCATCTCCAG |
| | | AGACAACGCCAAGAACACGCTGTATCTGCAGATGAACAGCCTGAG |
| | | AGCCGAGGACACGGCCGTGTATTACTGTGCGTCCCAATGGGGTGAT |
| | | TGGGGCCACTTTGAGTACTGGGGCCAGGGCACCCTGGTCACCGTCT |
| | | CCTCAGCTAGCGAGCAGAAATTAATCTCAGAGGAGGACCTGCCTAG |
| | | CGGTGGCGGAGGTTCTGGAGGTGGGGGTTCCTCACCCACTGAACC |
| | | AAGCTCCAAAACCGGTAACCCCAGACACCTGCATGTTCTGATTGGG |
| | | ACCTCAGTGGTCAAAATCCCTTTCACCATCCTCCTCTTCTTTCTCCT |
| | | TCATCGCTGGTGCTCCAACAAAAAAAATGCTGCTGTAATGGACCAA |
| | | GAGCCTGCAGGGAACAGAACAGTGAACAGCGAGGATTCTGATGAA |
| | | CAAGACCATCAGGAGGTGTCATACGCATAATGAGC |
| 692 | 4-P19_P20 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGCCACGAACTT |
| | | CTCTCTGTTAAAGCAAGCAGGAGACGTGGAAGAAAACCCCGGTCC |
| | | TAGGATGGCCTTACCAGTGACCGCCTTGCTCCTGCCGCTGGCCTTG |
| | | CTGCTCCACGCCGCCAGGCCGGGATCCCAGTCTGCGCTGACTCAAA |
| | | CGGCCTCAGTGTCTGGGTCCCTGGGCCAGAAGGTCACCATCTCCTG |
| | | CACTGGAAGCAGGTCCAACATCGGTGCAAATTATGTGGGCTGGTAT |
| | | CAACAGGTCCCAGGAATAGGCCCTAGAACCGTCATTTTTGGTTATA |
| | | ATTATCGACCTTCAGGGGTCCCCGATCGATTCTCTAGCTCCAAGTCA |
| | | GGCAATTCAGCCACCCTGACCATCTCTGGGCTCCAGGCTGAGGACG |
| | | AGGCTGAGTATTATTGTTCAACATGGGACAGCAGTCTCAACGCTTA |
| | | CGTGTTCGGCTCAGGGACCCAGCTGACCGTCCTCGGCGGTGGTTCC |
| | | TCTAGATCTTCCTCCTCTGGTGGCGGTGGCTCGGGCGGTGGTGGGG |
| | | AGGTGCCACTGGTGGAATCTGGGGGAGACCTGGTGAAGCCTGGGG |
| | | GGTCCTTGAGACTGTCCTGTGTGGCCTCTGGATTCACCCTCAGTAA |
| | | CTATGGCATGAGCTGGGTCCGTCAGTCTCCAGGGAAGGGGCTGCA |
| | | GTGGGTCGCAGCTATTAGGAGTGGTGGAAACACATACTACGCAGAC |
| | | GCTGTGAAGGGCCGATTCACCATCTCCAGAGACAACGCCAAGAAC |
| | | ACGCTGTATCTGCAGATGAACAGCCTGAGAGCCGAGGACACGGCC |
| | | GTGTATTACTGTGCGTCCCAATGGGGTGATTGGGGCCACTTTGAGTA |
| | | CTGGGGCCAGGGCACCCTGGTCACCGTCTCCTCAGCTAGCGAGCA |
| | | GAAATTAATCTCAGAGGAGGACCTGCCTAGCGGTGGCGGAGGTTCT |
| | | GGAGGTGGGGGTTCCTCACCCACTGAACCAAGCTCCAAAACCGGT |
| | | AACCCCAGACACCTGCATGTTCTGATTGGGACCTCAGTGGTCAAAA |
| | | TCCCTTTCACCATCCTCCTCTTCTTTCTCCTTCATCGCTGGTGCTCCA |
| | | ACAAAAAAAATGCTGCTGTAATGGACCAAGAGCCTGCAGGGAACA |
| | | GAACAGTGAACAGCGAGGATTCTGATGAACAAGACCATCAGGAGG |
| | | TGTCATACGCAGTCGAGGGCGGCGGAGCCACGAACTTCTCTCTGTT |
| | | AAAGCAAGCAGGAGACGTGGAAGAAAACCCCGGTCCTAGGATGG |
| | | CCTTACCAGTGACCGCCTTGCTCCTGCCGCTGGCCTTGCTGCTCCA |
| | | CGCCGCCAGGCCGGAGGTGCAGCTGCAGCAGTCTGGGGCTGAGCT |
| | | GGTGAAGCCTGGGGCCTCAGTGAAGATGTCCTGCAAGGCTTCTGG |
| | | CTACACATTTACCAGTTACAATATGCACTGGGTAAAGCAGACACCT |
| | | GGACAGGGCCTGGAATGGATTGGAGCTATTTATCCAGGAAATGGTG |
| | | ATACTTCCTACAATCAGAAGTTCAAAGGCAAGGCCACATTGACTGC |
| | | AGACAAATCCTCCAGCACAGCCTACATGCAGCTCAGCAGCCTGAC |
| | | ATCTGAGGACTCTGCGGACTATTACTGTGCAAGATCTAATTATTACG |
| | | GTAGTAGCTACTGGTTCTTCGATGTCTGGGGCGCAGGGACCACGGT |
| | | CACCGTCTCCTCAGGCAGTACTAGCGGTGGTGGCTCCGGGGGCGGT |
| | | TCCGGTGGGGGCGGCAGCAGCGACATTGTGCTGACCCAATCTCCA |
| | | GCTATCCTGTCTGCATCTCCAGGGGAGAAGGTCACAATGACTTGCA |
| | | GGGCCAGCTCAAGTGTAAATTACATGGACTGGTACCAGAAGAAGC |
| | | CAGGTTCCTCCCCCAAACCCTGGATTTATGCCACATCCAACCTGGCT |
| | | TCTGGAGTCCCTGCTCGCTTCAGTGGCAGTGGGTCTGGGACCTCTT |
| | | ACTCTCTCACAATCAGCAGAGTGGAGGCTGAAGATGCTGCCACTTA |
| | | TTACTGCCAGCAGTGGAGTTTTAATCCACCCACGTTCGGAGGGGGG |
| | | ACCAAGCTGGAAATAAAAGGAGCTAGCCACCATCACCATCACCATC |
| | | CTAGCGGTGGCGGAGGTTCTGGAGGTGGGGGTTCCTCACCCACTG |
| | | AACCAAGCTCCAAAACCGGTAACCCCAGACACCTGCATGTTCTGAT |
| | | TGGGACCTCAGTGGTCAAAATCCCTTTCACCATCCTCCTCTTCTTTC |
| | | TCCTTCATCGCTGGTGCTCCAACAAAAAAAATGCTGCTGTAATGGA |
| | | CCAAGAGCCTGCAGGGAACAGAACAGTGAACAGCGAGGATTCTGA |
| | | TGAACAAGACCATCAGGAGGTGTCATACGCATAATGAGC |
| 693 | 5-T20_T19 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGAGGGCAGAG |
| | | GAAGTCTTCTAACATGCGGTGACGTGGAGGAGAATCCCGGCCCTAG |
| | | GATGGCCTTACCAGTGACCGCCTTGCTCCTGCCGCTGGCCTTGCTG |
| | | CTCCACGCCGCCAGGCCGGAGGTGCAGCTGCAGCAGTCTGGGGCT |
| | | GAGCTGGTGAAGCCTGGGGCCTCAGTGAAGATGTCCTGCAAGGCT |
| | | TCTGGCTACACATTTACCAGTTACAATATGCACTGGGTAAAGCAGAC |
| | | ACCTGGACAGGGCCTGGAATGGATTGGAGCTATTTATCCAGGAAAT |
| | | GGTGATACTTCCTACAATCAGAAGTTCAAAGGCAAGGCCACATTGA |
| | | CTGCAGACAAATCCTCCAGCACAGCCTACATGCAGCTCAGCAGCCT |
| | | GACATCTGAGGACTCTGCGGACTATTACTGTGCAAGATCTAATTATT |
| | | ACGGTAGTAGCTACTGGTTCTTCGATGTCTGGGGCGCAGGGACCAC |
| | | GGTCACCGTCTCCTCAGGCAGTACTAGCGGTGGTGGCTCCGGGGGC |
| | | GGTTCCGGTGGGGGCGGCAGCAGCGACATTGTGCTGACCCAATCTC |
| | | CAGCTATCCTGTCTGCATCTCCAGGGGAGAAGGTCACAATGACTTG |
| | | CAGGGCCAGCTCAAGTGTAAATTACATGGACTGGTACCAGAAGAA |
| | | GCCAGGTTCCTCCCCCAAACCCTGGATTTATGCCACATCCAACCTG |
| | | GCTTCTGGAGTCCCTGCTCGCTTCAGTGGCAGTGGGTCTGGGACCT |
| | | CTTACTCTCTCACAATCAGCAGAGTGGAGGCTGAAGATGCTGCCAC |
| | | TTATTACTGCCAGCAGTGGAGTTTTAATCCACCCACGTTCGGAGGG |
| | | GGGACCAAGCTGGAAATAAAAGGAGCTAGCCACCATCACCATCAC |
| | | CATCCTAGCGGTGGCGGAGGTTCTGGAGGTGGGGGTTCCTCACCCA |
| | | CTGAACCAAGCTCCAAAACCGGTAACCCCAGACACCTGCATGTTCT |
| | | GATTGGGACCTCAGTGGTCAAAATCCCTTTCACCATCCTCCTCTTCT |
| | | TTCTCCTTCATCGCTGGTGCTCCAACAAAAAAAATGCTGCTGTAAT |
| | | GGACCAAGAGCCTGCAGGGAACAGAACAGTGAACAGCGAGGATT |
| | | CTGATGAACAAGACCATCAGGAGGTGTCATACGCAGTCGAGGGCG |
| | | GCGGAGAGGGCAGAGGAAGTCTTCTAACATGCGGTGACGTGGAGG |
| | | AGAATCCCGGCCCTAGGATGGCCTTACCAGTGACCGCCTTGCTCCT |
| | | GCCGCTGGCCTTGCTGCTCCACGCCGCCAGGCCGGGATCCCAGTCT |
| | | GCGCTGACTCAAACGGCCTCAGTGTCTGGGTCCCTGGGCCAGAAG |
| | | GTCACCATCTCCTGCACTGGAAGCAGGTCCAACATCGGTGCAAATT |
| | | ATGTGGGCTGGTATCAACAGGTCCCAGGAATAGGCCCTAGAACCGT |
| | | CATTTTTGGTTATAATTATCGACCTTCAGGGGTCCCCGATCGATTCTC |
| | | TAGCTCCAAGTCAGGCAATTCAGCCACCCTGACCATCTCTGGGCTC |
| | | CAGGCTGAGGACGAGGCTGAGTATTATTGTTCAACATGGGACAGCA |
| | | GTCTCAACGCTTACGTGTTCGGCTCAGGGACCCAGCTGACCGTCCT |
| | | CGGCGGTGGTTCCTCTAGATCTTCCTCCTCTGGTGGCGGTGGCTCG |
| | | GGCGGTGGTGGGGAGGTGCCACTGGTGGAATCTGGGGGAGACCTG |
| | | GTGAAGCCTGGGGGGTCCTTGAGACTGTCCTGTGTGGCCTCTGGAT |
| | | TCACCCTCAGTAACTATGGCATGAGCTGGGTCCGTCAGTCTCCAGG |
| | | GAAGGGGCTGCAGTGGGTCGCAGCTATTAGGAGTGGTGGAAACAC |
| | | ATACTACGCAGACGCTGTGAAGGGCCGATTCACCATCTCCAGAGAC |
| | | AACGCCAAGAACACGCTGTATCTGCAGATGAACAGCCTGAGAGCC |
| | | GAGGACACGGCCGTGTATTACTGTGCGTCCCAATGGGGTGATTGGG |
| | | GCCACTTTGAGTACTGGGGCCAGGGCACCCTGGTCACCGTCTCCTC |
| | | AGCTAGCGAGCAGAAATTAATCTCAGAGGAGGACCTGCCTAGCGG |
| | | TGGCGGAGGTTCTGGAGGTGGGGGTTCCTCACCCACTGAACCAAG |
| | | CTCCAAAACCGGTAACCCCAGACACCTGCATGTTCTGATTGGGACC |
| | | TCAGTGGTCAAAATCCCTTTCACCATCCTCCTCTTCTTTCTCCTTCAT |
| | | CGCTGGTGCTCCAACAAAAAAAATGCTGCTGTAATGGACCAAGAG |
| | | CCTGCAGGGAACAGAACAGTGAACAGCGAGGATTCTGATGAACAA |
| | | GACCATCAGGAGGTGTCATACGCATAATGAGC |
| 694 | 6-T19_T20 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGAGGGCAGAG |
| | | GAAGTCTTCTAACATGCGGTGACGTGGAGGAGAATCCCGGCCCTAG |
| | | GATGGCCTTACCAGTGACCGCCTTGCTCCTGCCGCTGGCCTTGCTG |
| | | CTCCACGCCGCCAGGCCGGGATCCCAGTCTGCGCTGACTCAAACG |
| | | GCCTCAGTGTCTGGGTCCCTGGGCCAGAAGGTCACCATCTCCTGCA |
| | | CTGGAAGCAGGTCCAACATCGGTGCAAATTATGTGGGCTGGTATCA |
| | | ACAGGTCCCAGGAATAGGCCCTAGAACCGTCATTTTTGGTTATAATT |
| | | ATCGACCTTCAGGGGTCCCCGATCGATTCTCTAGCTCCAAGTCAGG |
| | | CAATTCAGCCACCCTGACCATCTCTGGGCTCCAGGCTGAGGACGAG |
| | | GCTGAGTATTATTGTTCAACATGGGACAGCAGTCTCAACGCTTACGT |
| | | GTTCGGCTCAGGGACCCAGCTGACCGTCCTCGGCGGTGGTTCCTCT |
| | | AGATCTTCCTCCTCTGGTGGCGGTGGCTCGGGCGGTGGTGGGGAG |
| | | GTGCCACTGGTGGAATCTGGGGGAGACCTGGTGAAGCCTGGGGGG |
| | | TCCTTGAGACTGTCCTGTGTGGCCTCTGGATTCACCCTCAGTAACTA |
| | | TGGCATGAGCTGGGTCCGTCAGTCTCCAGGGAAGGGGCTGCAGTG |
| | | GGTCGCAGCTATTAGGAGTGGTGGAAACACATACTACGCAGACGCT |
| | | GTGAAGGGCCGATTCACCATCTCCAGAGACAACGCCAAGAACACG |
| | | CTGTATCTGCAGATGAACAGCCTGAGAGCCGAGGACACGGCCGTG |
| | | TATTACTGTGCGTCCCAATGGGGTGATTGGGGCCACTTTGAGTACTG |
| | | GGGCCAGGGCACCCTGGTCACCGTCTCCTCAGCTAGCGAGCAGAA |
| | | ATTAATCTCAGAGGAGGACCTGCCTAGCGGTGGCGGAGGTTCTGGA |
| | | GGTGGGGGTTCCTCACCCACTGAACCAAGCTCCAAAACCGGTAAC |
| | | CCCAGACACCTGCATGTTCTGATTGGGACCTCAGTGGTCAAAATCC |
| | | CTTTCACCATCCTCCTCTTCTTTCTCCTTCATCGCTGGTGCTCCAAC |
| | | AAAAAAAATGCTGCTGTAATGGACCAAGAGCCTGCAGGGAACAGA |
| | | ACAGTGAACAGCGAGGATTCTGATGAACAAGACCATCAGGAGGTG |
| | | TCATACGCAGTCGAGGGCGGCGGAGAGGGCAGAGGAAGTCTTCTA |
| | | ACATGCGGTGACGTGGAGGAGAATCCCGGCCCTAGGATGGCCTTAC |
| | | CAGTGACCGCCTTGCTCCTGCCGCTGGCCTTGCTGCTCCACGCCGC |
| | | CAGGCCGGAGGTGCAGCTGCAGCAGTCTGGGGCTGAGCTGGTGAA |
| | | GCCTGGGGCCTCAGTGAAGATGTCCTGCAAGGCTTCTGGCTACACA |
| | | TTTACCAGTTACAATATGCACTGGGTAAAGCAGACACCTGGACAGG |
| | | GCCTGGAATGGATTGGAGCTATTTATCCAGGAAATGGTGATACTTCC |
| | | TACAATCAGAAGTTCAAAGGCAAGGCCACATTGACTGCAGACAAA |
| | | TCCTCCAGCACAGCCTACATGCAGCTCAGCAGCCTGACATCTGAGG |
| | | ACTCTGCGGACTATTACTGTGCAAGATCTAATTATTACGGTAGTAGC |
| | | TACTGGTTCTTCGATGTCTGGGGCGCAGGGACCACGGTCACCGTCT |
| | | CCTCAGGCAGTACTAGCGGTGGTGGCTCCGGGGGCGGTTCCGGTG |
| | | GGGGCGGCAGCAGCGACATTGTGCTGACCCAATCTCCAGCTATCCT |
| | | GTCTGCATCTCCAGGGGAGAAGGTCACAATGACTTGCAGGGCCAG |
| | | CTCAAGTGTAAATTACATGGACTGGTACCAGAAGAAGCCAGGTTCC |
| | | TCCCCCAAACCCTGGATTTATGCCACATCCAACCTGGCTTCTGGAGT |
| | | CCCTGCTCGCTTCAGTGGCAGTGGGTCTGGGACCTCTTACTCTCTC |
| | | ACAATCAGCAGAGTGGAGGCTGAAGATGCTGCCACTTATTACTGCC |
| | | AGCAGTGGAGTTTTAATCCACCCACGTTCGGAGGGGGGACCAAGC |
| | | TGGAAATAAAAGGAGCTAGCCACCATCACCATCACCATCCTAGCGG |
| | | TGGCGGAGGTTCTGGAGGTGGGGGTTCCTCACCCACTGAACCAAG |
| | | CTCCAAAACCGGTAACCCCAGACACCTGCATGTTCTGATTGGGACC |
| | | TCAGTGGTCAAAATCCCTTTCACCATCCTCCTCTTCTTTCTCCTTCAT |
| | | CGCTGGTGCTCCAACAAAAAAAATGCTGCTGTAATGGACCAAGAG |
| | | CCTGCAGGGAACAGAACAGTGAACAGCGAGGATTCTGATGAACAA |
| | | GACCATCAGGAGGTGTCATACGCATAATGAGC |
| 695 | 7-P20_T19 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGCCACGAACTT |
| | | CTCTCTGTTAAAGCAAGCAGGAGACGTGGAAGAAAACCCCGGTCC |
| | | TAGGATGGCCTTACCAGTGACCGCCTTGCTCCTGCCGCTGGCCTTG |
| | | CTGCTCCACGCCGCCAGGCCGGAGGTGCAGCTGCAGCAGTCTGGG |
| | | GCTGAGCTGGTGAAGCCTGGGGCCTCAGTGAAGATGTCCTGCAAG |
| | | GCTTCTGGCTACACATTTACCAGTTACAATATGCACTGGGTAAAGCA |
| | | GACACCTGGACAGGGCCTGGAATGGATTGGAGCTATTTATCCAGGA |
| | | AATGGTGATACTTCCTACAATCAGAAGTTCAAAGGCAAGGCCACAT |
| | | TGACTGCAGACAAATCCTCCAGCACAGCCTACATGCAGCTCAGCAG |
| | | CCTGACATCTGAGGACTCTGCGGACTATTACTGTGCAAGATCTAATT |
| | | ATTACGGTAGTAGCTACTGGTTCTTCGATGTCTGGGGCGCAGGGAC |
| | | CACGGTCACCGTCTCCTCAGGCAGTACTAGCGGTGGTGGCTCCGGG |
| | | GGCGGTTCCGGTGGGGGCGGCAGCAGCGACATTGTGCTGACCCAA |
| | | TCTCCAGCTATCCTGTCTGCATCTCCAGGGGAGAAGGTCACAATGA |
| | | CTTGCAGGGCCAGCTCAAGTGTAAATTACATGGACTGGTACCAGAA |
| | | GAAGCCAGGTTCCTCCCCCAAACCCTGGATTTATGCCACATCCAAC |
| | | CTGGCTTCTGGAGTCCCTGCTCGCTTCAGTGGCAGTGGGTCTGGGA |
| | | CCTCTTACTCTCTCACAATCAGCAGAGTGGAGGCTGAAGATGCTGC |
| | | CACTTATTACTGCCAGCAGTGGAGTTTTAATCCACCCACGTTCGGA |
| | | GGGGGGACCAAGCTGGAAATAAAAGGAGCTAGCCACCATCACCAT |
| | | CACCATCCTAGCGGTGGCGGAGGTTCTGGAGGTGGGGGTTCCTCAC |
| | | CCACTGAACCAAGCTCCAAAACCGGTAACCCCAGACACCTGCATG |
| | | TTCTGATTGGGACCTCAGTGGTCAAAATCCCTTTCACCATCCTCCTC |
| | | TTCTTTCTCCTTCATCGCTGGTGCTCCAACAAAAAAAATGCTGCTGT |
| | | AATGGACCAAGAGCCTGCAGGGAACAGAACAGTGAACAGCGAGG |
| | | ATTCTGATGAACAAGACCATCAGGAGGTGTCATACGCAGTCGAGGG |
| | | CGGCGGAGAGGGCAGAGGAAGTCTTCTAACATGCGGTGACGTGGA |
| | | GGAGAATCCCGGCCCTAGGATGGCCTTACCAGTGACCGCCTTGCTC |
| | | CTGCCGCTGGCCTTGCTGCTCCACGCCGCCAGGCCGGGATCCCAGT |
| | | CTGCGCTGACTCAAACGGCCTCAGTGTCTGGGTCCCTGGGCCAGA |
| | | AGGTCACCATCTCCTGCACTGGAAGCAGGTCCAACATCGGTGCAA |
| | | ATTATGTGGGCTGGTATCAACAGGTCCCAGGAATAGGCCCTAGAAC |
| | | CGTCATTTTTGGTTATAATTATCGACCTTCAGGGGTCCCCGATCGATT |
| | | CTCTAGCTCCAAGTCAGGCAATTCAGCCACCCTGACCATCTCTGGG |
| | | CTCCAGGCTGAGGACGAGGCTGAGTATTATTGTTCAACATGGGACA |
| | | GCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCT |
| | | CGGGCGGTGGTGGGGAGGTGCCACTGGTGGAATCTGGGGGAGACC |
| | | TGGTGAAGCCTGGGGGGTCCTTGAGACTGTCCTGTGTGGCCTCTGG |
| | | ATTCACCCTCAGTAACTATGGCATGAGCTGGGTCCGTCAGTCTCCA |
| | | GGGAAGGGGCTGCAGTGGGTCGCAGCTATTAGGAGTGGTGGAAAC |
| | | ACATACTACGCAGACGCTGTGAAGGGCCGATTCACCATCTCCAGAG |
| | | ACAACGCCAAGAACACGCTGTATCTGCAGATGAACAGCCTGAGAG |
| | | CCGAGGACACGGCCGTGTATTACTGTGCGTCCCAATGGGGTGATTG |
| | | GGGCCACTTTGAGTACTGGGGCCAGGGCACCCTGGTCACCGTCTCC |
| | | TCAGCTAGCGAGCAGAAATTAATCTCAGAGGAGGACCTGCCTAGCG |
| | | GTGGCGGAGGTTCTGGAGGTGGGGGTTCCTCACCCACTGAACCAA |
| | | GCTCCAAAACCGGTAACCCCAGACACCTGCATGTTCTGATTGGGAC |
| | | CTCAGTGGTCAAAATCCCTTTCACCATCCTCCTCTTCTTTCTCCTTC |
| | | ATCGCTGGTGCTCCAACAAAAAAAATGCTGCTGTAATGGACCAAGA |
| | | GCCTGCAGGGAACAGAACAGTGAACAGCGAGGATTCTGATGAACA |
| | | AGACCATCAGGAGGTGTCATACGCATAATGAGC |
| 696 | 8-P19_T20 | ATGGGGGGACTTGAACCCTGCAGCAGGTTCCTGCTCCTGCCTCTCC |
| | DNA | TGCTGGCTGTAAGTGGTCTCCGTCCTGTCCAGGTCCAGGCCCAGAG |
| | | CGATTGCAGTTGCTCTACGGTGAGCCCGGGCGTGCTGGCAGGGATC |
| | | GTGATGGGAGACCTGGTGCTGACAGTGCTCATTGCCCTGGCCGTGT |
| | | ACTTCCTGGGCCGGCTGGTCCCTCGGGGGCGAGGGGCTGCGGAGG |
| | | CAGCGACCCGGAAACAGCGTATCACTGAGACCGAGTCGCCTTATCA |
| | | GGAGCTCCAGGGTCAGAGGTCGGATGTCTACAGCGACCTCAACAC |
| | | ACAGAGGCCGTATTACAAAGTCGAGGGCGGCGGAGCCACGAACTT |
| | | CTCTCTGTTAAAGCAAGCAGGAGACGTGGAAGAAAACCCCGGTCC |
| | | TAGGATGGCCTTACCAGTGACCGCCTTGCTCCTGCCGCTGGCCTTG |
| | | CTGCTCCACGCCGCCAGGCCGGGATCCCAGTCTGCGCTGACTCAAA |
| | | CGGCCTCAGTGTCTGGGTCCCTGGGCCAGAAGGTCACCATCTCCTG |
| | | CACTGGAAGCAGGTCCAACATCGGTGCAAATTATGTGGGCTGGTAT |
| | | CAACAGGTCCCAGGAATAGGCCCTAGAACCGTCATTTTTGGTTATA |
| | | ATTATCGACCTTCAGGGGTCCCCGATCGATTCTCTAGCTCCAAGTCA |
| | | GGCAATTCAGCCACCCTGACCATCTCTGGGCTCCAGGCTGAGGACG |
| | | AGGCTGAGTATTATTGTTCAACATGGGACAGCAGTCTCAACGCTTA |
| | | CGTGTTCGGCTCAGGGACCCAGCTGACCGTCCTCGGCGGTGGTTCC |
| | | TCTAGATCTTCCTCCTCTGGTGGCGGTGGCTCGGGCGGTGGTGGGG |
| | | AGGTGCCACTGGTGGAATCTGGGGGAGACCTGGTGAAGCCTGGGG |
| | | GGTCCTTGAGACTGTCCTGTGTGGCCTCTGGATTCACCCTCAGTAA |
| | | CTATGGCATGAGCTGGGTCCGTCAGTCTCCAGGGAAGGGGCTGCA |
| | | GTGGGTCGCAGCTATTAGGAGTGGTGGAAACACATACTACGCAGAC |
| | | GCTGTGAAGGGCCGATTCACCATCTCCAGAGACAACGCCAAGAAC |
| | | ACGCTGTATCTGCAGATGAACAGCCTGAGAGCCGAGGACACGGCC |
| | | GTGTATTACTGTGCGTCCCAATGGGGTGATTGGGGCCACTTTGAGTA |
| | | CTGGGGCCAGGGCACCCTGGTCACCGTCTCCTCAGCTAGCGAGCA |
| | | GAAATTAATCTCAGAGGAGGACCTGCCTAGCGGTGGCGGAGGTTCT |
| | | GGAGGTGGGGGTTCCTCACCCACTGAACCAAGCTCCAAAACCGGT |
| | | AACCCCAGACACCTGCATGTTCTGATTGGGACCTCAGTGGTCAAAA |
| | | TCCCTTTCACCATCCTCCTCTTCTTTCTCCTTCATCGCTGGTGCTCCA |
| | | ACAAAAAAAATGCTGCTGTAATGGACCAAGAGCCTGCAGGGAACA |
| | | GAACAGTGAACAGCGAGGATTCTGATGAACAAGACCATCAGGAGG |
| | | TGTCATACGCAGTCGAGGGCGGCGGAGAGGGCAGAGGAAGTCTTC |
| | | TAACATGCGGTGACGTGGAGGAGAATCCCGGCCCTAGGATGGCCTT |
| | | ACCAGTGACCGCCTTGCTCCTGCCGCTGGCCTTGCTGCTCCACGCC |
| | | GCCAGGCCGGAGGTGCAGCTGCAGCAGTCTGGGGCTGAGCTGGTG |
| | | AAGCCTGGGGCCTCAGTGAAGATGTCCTGCAAGGCTTCTGGCTACA |
| | | CATTTACCAGTTACAATATGCACTGGGTAAAGCAGACACCTGGACA |
| | | GGGCCTGGAATGGATTGGAGCTATTTATCCAGGAAATGGTGATACTT |
| | | CCTACAATCAGAAGTTCAAAGGCAAGGCCACATTGACTGCAGACA |
| | | AATCCTCCAGCACAGCCTACATGCAGCTCAGCAGCCTGACATCTGA |
| | | GGACTCTGCGGACTATTACTGTGCAAGATCTAATTATTACGGTAGTA |
| | | GCTACTGGTTCTTCGATGTCTGGGGCGCAGGGACCACGGTCACCGT |
| | | CTCCTCAGGCAGTACTAGCGGTGGTGGCTCCGGGGGCGGTTCCGGT |
| | | GGGGGCGGCAGCAGCGACATTGTGCTGACCCAATCTCCAGCTATCC |
| | | TGTCTGCATCTCCAGGGGAGAAGGTCACAATGACTTGCAGGGCCA |
| | | GCTCAAGTGTAAATTACATGGACTGGTACCAGAAGAAGCCAGGTTC |
| | | CTCCCCCAAACCCTGGATTTATGCCACATCCAACCTGGCTTCTGGA |
| | | GTCCCTGCTCGCTTCAGTGGCAGTGGGTCTGGGACCTCTTACTCTC |
| | | TCACAATCAGCAGAGTGGAGGCTGAAGATGCTGCCACTTATTACTG |
| | | CCAGCAGTGGAGTTTTAATCCACCCACGTTCGGAGGGGGGACCAA |
| | | GCTGGAAATAAAAGGAGCTAGCCACCATCACCATCACCATCCTAGC |
| | | GGTGGCGGAGGTTCTGGAGGTGGGGGTTCCTCACCCACTGAACCA |
| | | AGCTCCAAAACCGGTAACCCCAGACACCTGCATGTTCTGATTGGGA |
| | | CCTCAGTGGTCAAAATCCCTTTCACCATCCTCCTCTTCTTTCTCCTT |
| | | CATCGCTGGTGCTCCAACAAAAAAAATGCTGCTGTAATGGACCAAG |
| | | AGCCTGCAGGGAACAGAACAGTGAACAGCGAGGATTCTGATGAAC |
| | | AAGACCATCAGGAGGTGTCATACGCATAATGAGC |

In Table 11, elements of each construct include the following. 1-T19_P20—DAP12/T2A cleavage site/DS191 binder/KIR2DS2 fragment/P2A cleavage site/Leu16 binder/KIR2DS2 fragment; 2-T20_P19—DAP12/T2A cleavage site/Leu16 binder/KIR2DS2 fragment/P2A cleavage site/DS191 binder/KIR2DS2 fragment; 3-P20_P19—DAP12/P2A cleavage site/Leu16 binder/KIR2DS2 fragment/P2A cleavage site/DS191 binder/KIR2DS2 fragment; 4-P19_P20—DAP12/P2A cleavage site/DS191 binder/KIR2DS2 fragment/P2A cleavage site/Leu16 binder/KIR2DS2 fragment; 5-T20_T19—DAP12/T2A cleavage site/Leu16 binder/KIR2DS2 fragment/T2A cleavage site/DS191 binder/KIR2DS2 fragment; 6-T19_T20—DAP12/T2A cleavage site/DS191 binder/KIR2DS2 fragment/T2A cleavage site/Leu16 binder/KIR2DS2 fragment; 7-P20_T19—DAP12/P2A cleavage site/Leu16 binder/KIR2DS2 fragment/T2A cleavage site/DS191 binder/KIR2DS2 fragment; and 8-P19_T20—DAP12/P2A cleavage site/DS191 binder/KIR2DS2 fragment/T2A cleavage site/Leu16 binder/KIR2DS2 fragment. ADS191 binder is an anti-CD19 binder and Leu16 is an anti-CD20 binder.

Further exemplary fusion proteins of embodiments herein are set forth in Table 8. Some embodiments comprise a nucleic acid sequence encoding one of SEQ ID NOS: 708-723, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 708-723. In some embodiments, the nucleic acid sequence of the present disclosure comprises the amino acid sequence of SEQ ID NO: 720 or a variant thereof having at least about 70%, about 75%, about 80%, about 85%, about 90%, about 91%, about 92%, about 93%, about 94%, about 95%, about 96%, about 97%, about 98%, about 99% or more sequence identity thereto.

Some embodiments comprise a nucleic acid sequence comprising one of SEQ ID NOS: 724-739 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 724-739. In some embodiments, the nucleic acid sequence of the present disclosure comprises the sequence of SEQ ID NO: 810 or a variant thereof having at least about 70%, about 75%, about 80%, about 85%, about 90%, about 91%, about 92%, about 93%, about 94%, about 95%, about 96%, about 97%, about 98%, about 99% or more sequence identity thereto.

TABLE 12
Further Exemplary Fusion Proteins

| SEQ | | |
|---|---|---|
| ID. | | |
| NO. | Name | Sequence |
| 708 | 9 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PGSQSALTQTASVSGSLGQKVTISCTGSRSNIGANYVGWYQQVPGIGPRTVIF |
| | | GYNYRPSGVPDRFSSSKSGNSATLTISGLQAEDEAEYYCSTWDSSLNAYVFGS |
| | | GTQLTVLGGGSSRSSSSGGGGSGGGGEVPLVESGGDLVKPGGSLRLSCVASGF |
| | | TLSNYGMSWVRQSPGKGLQWVAAIRSGGNTYYADAVKGRFTISRDNAKNTL |
| | | YLQMNSLRAEDTAVYYCASQWGDWGHFEYWGQGTLVTVSSASEQKLISEED |
| | | LPSGGGGSGGGGSSPTEPSSKTGNPRHLHVLIGTSVVKIPFTILLFFLLHRWCS |
| | | NKKNAAVMDQEPAGNRTVNSEDSDEQDHQEVSYA |
| 709 | 10 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PEVQLQQSGAELVKPGASVKMSCKASGYTFTSYNMHWVKQTPGQGLEWIG |
| | | AIYPGNGDTSYNQKFKGKATLTADKSSSTAYMQLSSLTSEDSADYYCARSNY |
| | | YGSSYWFFDVWGAGTTVTVSSGSTSGGGSGGGSGGGGSSDIVLTQSPAILSAS |
| | | PGEKVTMTCRASSSVNYMDWYQKKPGSSPKPWIYATSNLASGVPARFSGSGS |
| | | GTSYSLTISRVEAEDAATYYCQQWSFNPPTFGGGTKLEIKGASHHHHHHPSGG |
| | | GGSGGGGSSPTEPSSKTGNPRHLHVLIGTSVVKIPFTILLFFLLHRWCSNKKNA |
| | | AVMDQEPAGNRTVNSEDSDEQDHQEVSYA |
| 710 | 11 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PEVQLQQSGAELVKPGASVKMSCKASGYTFTSYNMHWVKQTPGQGLEWIG |
| | | AIYPGNGDTSYNQKFKGKATLTADKSSSTAYMQLSSLTSEDSADYYCARSNY |
| | | YGSSYWFFDVWGAGTTVTVSSGSTSGGGSGGGSGGGGSSDIVLTQSPAILSAS |
| | | PGEKVTMTCRASSSVNYMDWYQKKPGSSPKPWIYATSNLASGVPARFSGSGS |
| | | GTSYSLTISRVEAEDAATYYCQQWSFNPPTFGGGTKLEIKPSGGGGSGGGGSG |
| | | GGGSGGGGSGGGGSSPTEPSSKTGNPRHLHVLIGTSVVKIPFTILLFFLLHRWC |
| | | SNKKNAAVMDQEPAGNRTVNSEDSDEQDHQEVSYA |
| 711 | 12 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PEVQLQQSGAELVKPGASVKMSCKASGYTFTSYNMHWVKQTPGQGLEWIG |
| | | AIYPGNGDTSYNQKFKGKATLTADKSSSTAYMQLSSLTSEDSADYYCARSNY |
| | | YGSSYWFFDVWGAGTTVTVSSGSTSGGGSGGGSGGGGSSDIVLTQSPAILSAS |
| | | PGEKVTMTCRASSSVNYMDWYQKKPGSSPKPWIYATSNLASGVPARFSGSGS |
| | | GTSYSLTISRVEAEDAATYYCQQWSFNPPTFGGGTKLEIKPSGGGGSGGGGSG |
| | | GGGSGGGGSGGGGSPYEWSNSSDPLLVSVTGNPSNSWPSPTEPSSKTGNPRH |
| | | LHVLIGTSVVKIPFTILLFFLLHRWCSNKKNAAVMDQEPAGNRTVNSEDSDEQ |
| | | DHQEVSYA |
| 712 | 13 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PGSQSALTQTASVSGSLGQKVTISCTGSRSNIGANYVGWYQQVPGIGPRTVIF |
| | | GYNYRPSGVPDRFSSSKSGNSATLTISGLQAEDEAEYYCSTWDSSLNAYVFGS |
| | | GTQLTVLGGGSSRSSSSGGGGSGGGGEVPLVESGGDLVKPGGSLRLSCVASGF |
| | | TLSNYGMSWVRQSPGKGLQWVAAIRSGGNTYYADAVKGRFTISRDNAKNTL |
| | | YLQMNSLRAEDTAVYYCASQWGDWGHFEYWGQGTLVTVSSASEQKLISEED |
| | | LPSGGGGSGGGGSSPTEPSSKTGNPRHLHVLIGTSVVKIPFTILLFFLLHRWCS |
| | | NKKNAAVMDQEPAGNRTVNSEDSDEQDHQEVSYAVEGGGATNFSLLKQAGD |
| | | VEENPGPRMALPVTALLLPLALLLHAARPEVQLQQSGAELVKPGASVKMSCK |
| | | ASGYTFTSYNMHWVKQTPGQGLEWIGAIYPGNGDTSYNQKFKGKATLTADK |
| | | SSSTAYMQLSSLTSEDSADYYCARSNYYGSSYWFFDVWGAGTTVTVSSGSTS |
| | | GGGSGGGSGGGGSSDIVLTQSPAILSASPGEKVTMTCRASSSVNYMDWYQKK |
| | | PGSSPKPWIYATSNLASGVPARFSGSGSGTSYSLTISRVEAEDAATYYCQQWSF |
| | | NPPTFGGGTKLEIKGASHHHHHHPSGGGGSGGGGSSPTEPSSKTGNPRHLHV |
| | | LIGTSVVKIPFTILLFFLLHRWCSNKKNAAVMDQEPAGNRTVNSEDSDEQDHQ |
| | | EVSYA |
| 713 | 14 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PEVQLQQSGAELVKPGASVKMSCKASGYTFTSYNMHWVKQTPGQGLEWIG |
| | | AIYPGNGDTSYNQKFKGKATLTADKSSSTAYMQLSSLTSEDSADYYCARSNY |
| | | YGSSYWFFDVWGAGTTVTVSSGSTSGGGSGGGSGGGGSSDIVLTQSPAILSAS |
| | | PGEKVTMTCRASSSVNYMDWYQKKPGSSPKPWIYATSNLASGVPARFSGSGS |
| | | GTSYSLTISRVEAEDAATYYCQQWSFNPPTFGGGTKLEIKGASHHHHHHPSGG |
| | | GGSGGGGSSPTEPSSKTGNPRHLHVLIGTSVVKIPFTILLFFLLHRWCSNKKNA |
| | | AVMDQEPAGNRTVNSEDSDEQDHQEVSYAVEGGGATNFSLLKQAGDVEENP |
| | | GPRMALPVTALLLPLALLLHAARPGSQSALTQTASVSGSLGQKVTISCTGSRS |
| | | NIGANYVGWYQQVPGIGPRTVIFGYNYRPSGVPDRFSSSKSGNSATLTISGLQ |
| | | AEDEAEYYCSTWDSSLNAYVFGSGTQLTVLGGGSSRSSSSGGGGSGGGGEVP |
| | | LVESGGDLVKPGGSLRLSCVASGFTLSNYGMSWVRQSPGKGLQWVAAIRSGG |
| | | NTYYADAVKGRFTISRDNAKNTLYLQMNSLRAEDTAVYYCASQWGDWGHF |
| | | EYWGQGTLVTVSSASEQKLISEEDLPSGGGGSGGGGSSPTEPSSKTGNPRHLH |
| | | VLIGTSVVKIPFTILLFFLLHRWCSNKKNAAVMDQEPAGNRTVNSEDSDEQDH |
| | | QEVSYA |
| 714 | 15 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGATNFSLLKQAGDVEENPGPRMALPVTALLLPLALLLHA |
| | | ARPEVQLQQSGAELVKPGASVKMSCKASGYTFTSYNMHWVKQTPGQGLEW |
| | | IGAIYPGNGDTSYNQKFKGKATLTADKSSSTAYMQLSSLTSEDSADYYCARSN |
| | | YYGSSYWFFDVWGAGTTVTVSSGSTSGGGSGGGSGGGGSSDIVLTQSPAILS |
| | | ASPGEKVTMTCRASSSVNYMDWYQKKPGSSPKPWIYATSNLASGVPARFSGS |
| | | GSGTSYSLTISRVEAEDAATYYCQQWSFNPPTFGGGTKLEIKGASHHHHHHPS |
| | | GGGGSGGGGSSPTEPSSKTGNPRHLHVLIGTSVVKIPFTILLFFLLHRWCSNKK |
| | | NAAVMDQEPAGNRTVNSEDSDEQDHQEVSYAVEGGGATNFSLLKQAGDVEE |
| | | NPGPRMALPVTALLLPLALLLHAARPGSQSALTQTASVSGSLGQKVTISCTGS |
| | | RSNIGANYVGWYQQVPGIGPRTVIFGYNYRPSGVPDRESSSKSGNSATLTISGL |
| | | QAEDEAEYYCSTWDSSLNAYVFGSGTQLTVLGGGSSRSSSSGGGGSGGGGEV |
| | | PLVESGGDLVKPGGSLRLSCVASGFTLSNYGMSWVRQSPGKGLQWVAAIRSG |
| | | GNTYYADAVKGRFTISRDNAKNTLYLQMNSLRAEDTAVYYCASQWGDWGH |
| | | FEYWGQGTLVTVSSASEQKLISEEDLPSGGGGSGGGGSSPTEPSSKTGNPRHL |
| | | HVLIGTSVVKIPFTILLFFLLHRWCSNKKNAAVMDQEPAGNRTVNSEDSDEQD |
| | | HQEVSYA |
| 715 | 16 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGATNFSLLKQAGDVEENPGPRMALPVTALLLPLALLLHA |
| | | ARPGSQSALTQTASVSGSLGQKVTISCTGSRSNIGANYVGWYQQVPGIGPRTV |
| | | IFGYNYRPSGVPDRFSSSKSGNSATLTISGLQAEDEAEYYCSTWDSSLNAYVF |
| | | GSGTQLTVLGGGSSRSSSSGGGGSGGGGEVPLVESGGDLVKPGGSLRLSCVAS |
| | | GFTLSNYGMSWVRQSPGKGLQWVAAIRSGGNTYYADAVKGRFTISRDNAKN |
| | | TLYLQMNSLRAEDTAVYYCASQWGDWGHFEYWGQGTLVTVSSASEQKLISE |
| | | EDLPSGGGGSGGGGSSPTEPSSKTGNPRHLHVLIGTSVVKIPFTILLFFLLHRW |
| | | CSNKKNAAVMDQEPAGNRTVNSEDSDEQDHQEVSYAVEGGGATNFSLLKQA |
| | | GDVEENPGPRMALPVTALLLPLALLLHAARPEVQLQQSGAELVKPGASVKMS |
| | | CKASGYTFTSYNMHWVKQTPGQGLEWIGAIYPGNGDTSYNQKFKGKATLTA |
| | | DKSSSTAYMQLSSLTSEDSADYYCARSNYYGSSYWFFDVWGAGTTVTVSSGS |
| | | TSGGGSGGGSGGGGSSDIVLTQSPAILSASPGEKVTMTCRASSSVNYMDWYQ |
| | | KKPGSSPKPWIYATSNLASGVPARFSGSGSGTSYSLTISRVEAEDAATYYCQQ |
| | | WSFNPPTFGGGTKLEIKGASHHHHHHPSGGGGSGGGGSSPTEPSSKTGNPRH |
| | | LHVLIGTSVVKIPFTILLFFLLHRWCSNKKNAAVMDQEPAGNRTVNSEDSDEQ |
| | | DHQEVSYA |
| 716 | 17 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PEVQLQQSGAELVKPGASVKMSCKASGYTFTSYNMHWVKQTPGQGLEWIG |
| | | AIYPGNGDTSYNQKFKGKATLTADKSSSTAYMQLSSLTSEDSADYYCARSNY |
| | | YGSSYWFFDVWGAGTTVTVSSGSTSGGGSGGGSGGGGSSDIVLTQSPAILSAS |
| | | PGEKVTMTCRASSSVNYMDWYQKKPGSSPKPWIYATSNLASGVPARFSGSGS |
| | | GTSYSLTISRVEAEDAATYYCQQWSFNPPTFGGGTKLEIKGASHHHHHHPSGG |
| | | GGSGGGGSSPTEPSSKTGNPRHLHVLIGTSVVKIPFTILLFFLLHRWCSNKKNA |
| | | AVMDQEPAGNRTVNSEDSDEQDHQEVSYAVEGGGEGRGSLLTCGDVEENPGP |
| | | RMALPVTALLLPLALLLHAARPGSQSALTQTASVSGSLGQKVTISCTGSRSNIG |
| | | ANYVGWYQQVPGIGPRTVIFGYNYRPSGVPDRFSSSKSGNSATLTISGLQAED |
| | | EAEYYCSTWDSSLNAYVFGSGTQLTVLGGGSSRSSSSGGGGSGGGGEVPLVE |
| | | SGGDLVKPGGSLRLSCVASGFTLSNYGMSWVRQSPGKGLQWVAAIRSGGNT |
| | | YYADAVKGRFTISRDNAKNTLYLQMNSLRAEDTAVYYCASQWGDWGHFEY |
| | | WGQGTLVTVSSASEQKLISEEDLPSGGGGSGGGGSSPTEPSSKTGNPRHLHVL |
| | | IGTSVVKIPFTILLFFLLHRWCSNKKNAAVMDQEPAGNRTVNSEDSDEQDHQE |
| | | VSYA |
| 717 | 18 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PGSQSALTQTASVSGSLGQKVTISCTGSRSNIGANYVGWYQQVPGIGPRTVIF |
| | | GYNYRPSGVPDRFSSSKSGNSATLTISGLQAEDEAEYYCSTWDSSLNAYVFGS |
| | | GTQLTVLGGGSSRSSSSGGGGSGGGGEVPLVESGGDLVKPGGSLRLSCVASGF |
| | | TLSNYGMSWVRQSPGKGLQWVAAIRSGGNTYYADAVKGRFTISRDNAKNTL |
| | | YLQMNSLRAEDTAVYYCASQWGDWGHFEYWGQGTLVTVSSASEQKLISEED |
| | | LPSGGGGSGGGGSSPTEPSSKTGNPRHLHVLIGTSVVKIPFTILLFFLLHRWCS |
| | | NKKNAAVMDQEPAGNRTVNSEDSDEQDHQEVSYAVEGGGEGRGSLLTCGDV |
| | | EENPGPRMALPVTALLLPLALLLHAARPEVQLQQSGAELVKPGASVKMSCKA |
| | | SGYTFTSYNMHWVKQTPGQGLEWIGAIYPGNGDTSYNQKFKGKATLTADKS |
| | | SSTAYMQLSSLTSEDSADYYCARSNYYGSSYWFFDVWGAGTTVTVSSGSTSG |
| | | GGSGGGSGGGGSSDIVLTQSPAILSASPGEKVTMTCRASSSVNYMDWYQKKP |
| | | GSSPKPWIYATSNLASGVPARFSGSGSGTSYSLTISRVEAEDAATYYCQQWSFN |
| | | PPTFGGGTKLEIKGASHHHHHHPSGGGGSGGGGSSPTEPSSKTGNPRHLHVLI |
| | | GTSVVKIPFTILLFFLLHRWCSNKKNAAVMDQEPAGNRTVNSEDSDEQDHQE |
| | | VSYA |
| 718 | 19 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGATNFSLLKQAGDVEENPGPRMALPVTALLLPLALLLHA |
| | | ARPEVQLQQSGAELVKPGASVKMSCKASGYTFTSYNMHWVKQTPGQGLEW |
| | | IGAIYPGNGDTSYNQKFKGKATLTADKSSSTAYMQLSSLTSEDSADYYCARSN |
| | | YYGSSYWFFDVWGAGTTVTVSSGSTSGGGSGGGSGGGGSSDIVLTQSPAILS |
| | | ASPGEKVTMTCRASSSVNYMDWYQKKPGSSPKPWIYATSNLASGVPARFSGS |
| | | GSGTSYSLTISRVEAEDAATYYCQQWSFNPPTFGGGTKLEIKGASHHHHHHPS |
| | | GGGGSGGGGSSPTEPSSKTGNPRHLHVLIGTSVVKIPFTILLFFLLHRWCSNKK |
| | | NAAVMDQEPAGNRTVNSEDSDEQDHQEVSYAVEGGGEGRGSLLTCGDVEEN |
| | | PGPRMALPVTALLLPLALLLHAARPGSQSALTQTASVSGSLGQKVTISCTGSR |
| | | SNIGANYVGWYQQVPGIGPRTVIFGYNYRPSGVPDRESSSKSGNSATLTISGLQ |
| | | AEDEAEYYCSTWDSSLNAYVFGSGTQLTVLGGGSSRSSSSGGGGSGGGGEVP |
| | | LVESGGDLVKPGGSLRLSCVASGFTLSNYGMSWVRQSPGKGLQWVAAIRSGG |
| | | NTYYADAVKGRFTISRDNAKNTLYLQMNSLRAEDTAVYYCASQWGDWGHF |
| | | EYWGQGTLVTVSSASEQKLISEEDLPSGGGGSGGGGSSPTEPSSKTGNPRHLH |
| | | VLIGTSVVKIPFTILLFFLLHRWCSNKKNAAVMDQEPAGNRTVNSEDSDEQDH |
| | | QEVSYA |
| 719 | 20 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGATNFSLLKQAGDVEENPGPRMALPVTALLLPLALLLHA |
| | | ARPGSQSALTQTASVSGSLGQKVTISCTGSRSNIGANYVGWYQQVPGIGPRTV |
| | | IFGYNYRPSGVPDRFSSSKSGNSATLTISGLQAEDEAEYYCSTWDSSLNAYVF |
| | | GSGTQLTVLGGGSSRSSSSGGGGSGGGGEVPLVESGGDLVKPGGSLRLSCVAS |
| | | GFTLSNYGMSWVRQSPGKGLQWVAAIRSGGNTYYADAVKGRFTISRDNAKN |
| | | TLYLQMNSLRAEDTAVYYCASQWGDWGHFEYWGQGTLVTVSSASEQKLISE |
| | | EDLPSGGGGSGGGGSSPTEPSSKTGNPRHLHVLIGTSVVKIPFTILLFFLLHRW |
| | | CSNKKNAAVMDQEPAGNRTVNSEDSDEQDHQEVSYAVEGGGEGRGSLLTCG |
| | | DVEENPGPRMALPVTALLLPLALLLHAARPEVQLQQSGAELVKPGASVKMSC |
| | | KASGYTFTSYNMHWVKQTPGQGLEWIGAIYPGNGDTSYNQKFKGKATLTAD |
| | | KSSSTAYMQLSSLTSEDSADYYCARSNYYGSSYWFFDVWGAGTTVTVSSGST |
| | | SGGGSGGGSGGGGSSDIVLTQSPAILSASPGEKVTMTCRASSSVNYMDWYQK |
| | | KPGSSPKPWIYATSNLASGVPARFSGSGSGTSYSLTISRVEAEDAATYYCQQWS |
| | | FNPPTFGGGTKLEIKGASHHHHHHPSGGGGSGGGGSSPTEPSSKTGNPRHLH |
| | | VLIGTSVVKIPFTILLFFLLHRWCSNKKNAAVMDQEPAGNRTVNSEDSDEQDH |
| | | QEVSYA |
| 720 | 21 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PGSQSALTQTASVSGSLGQKVTISCTGSRSNIGANYVGWYQQVPGIGPRTVIF |
| | | GYNYRPSGVPDRFSSSKSGNSATLTISGLQAEDEAEYYCSTWDSSLNAYVFGS |
| | | GTQLTVLGGGSSRSSSSGGGGSGGGGEVPLVESGGDLVKPGGSLRLSCVASGF |
| | | TLSNYGMSWVRQSPGKGLQWVAAIRSGGNTYYADAVKGRFTISRDNAKNTL |
| | | YLQMNSLRAEDTAVYYCASQWGDWGHFEYWGQGTLVTVSSASEQKLISEED |
| | | LPSGGGGSGGGGSSPTEPSSKTGNPRHLHVLIGTSVVKIPFTILLFFLLHRWCS |
| | | NKKNAAVMDQEPAGNRTVNSEDSDEQDHQEVSYAVEGGGATNFSLLKQAGD |
| | | VEENPGPRMALPVTALLLPLALLLHAARPEVQLQQSGAELVKPGASVKMSCK |
| | | ASGYTFTSYNMHWVKQTPGQGLEWIGAIYPGNGDTSYNQKFKGKATLTADK |
| | | SSSTAYMQLSSLTSEDSADYYCARSNYYGSSYWFFDVWGAGTTVTVSSGSTS |
| | | GGGSGGGSGGGGSSDIVLTQSPAILSASPGEKVTMTCRASSSVNYMDWYQKK |
| | | PGSSPKPWIYATSNLASGVPARFSGSGSGTSYSLTISRVEAEDAATYYCQQWSF |
| | | NPPTFGGGTKLEIKPSGGGGSGGGGSGGGGSGGGGSGGGGSPYEWSNSSDPL |
| | | LVSVTGNPSNSWPSPTEPSSKTGNPRHLHVLIGTSVVKIPFTILLFFLLHRWCSN |
| | | KKNAAVMDQEPAGNRTVNSEDSDEQDHQEVSYA |
| 721 | 22 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGATNFSLLKQAGDVEENPGPRMALPVTALLLPLALLLHA |
| | | ARPGSQSALTQTASVSGSLGQKVTISCTGSRSNIGANYVGWYQQVPGIGPRTV |
| | | IFGYNYRPSGVPDRFSSSKSGNSATLTISGLQAEDEAEYYCSTWDSSLNAYVF |
| | | GSGTQLTVLGGGSSRSSSSGGGGSGGGGEVPLVESGGDLVKPGGSLRLSCVAS |
| | | GFTLSNYGMSWVRQSPGKGLQWVAAIRSGGNTYYADAVKGRFTISRDNAKN |
| | | TLYLQMNSLRAEDTAVYYCASQWGDWGHFEYWGQGTLVTVSSASEQKLISE |
| | | EDLPSGGGGSGGGGSSPTEPSSKTGNPRHLHVLIGTSVVKIPFTILLFFLLHRW |
| | | CSNKKNAAVMDQEPAGNRTVNSEDSDEQDHQEVSYAVEGGGEGRGSLLTCG |
| | | DVEENPGPRMALPVTALLLPLALLLHAARPEVQLQQSGAELVKPGASVKMSC |
| | | KASGYTFTSYNMHWVKQTPGQGLEWIGAIYPGNGDTSYNQKFKGKATLTAD |
| | | KSSSTAYMQLSSLTSEDSADYYCARSNYYGSSYWFFDVWGAGTTVTVSSGST |
| | | SGGGSGGGSGGGGSSDIVLTQSPAILSASPGEKVTMTCRASSSVNYMDWYQK |
| | | KPGSSPKPWIYATSNLASGVPARFSGSGSGTSYSLTISRVEAEDAATYYCQQWS |
| | | FNPPTFGGGTKLEIKPSGGGGSGGGGSGGGGSGGGGSGGGGSPYEWSNSSDP |
| | | LLVSVTGNPSNSWPSPTEPSSKTGNPRHLHVLIGTSVVKIPFTILLFFLLHRWCS |
| | | NKKNAAVMDQEPAGNRTVNSEDSDEQDHQEVSYA |
| 722 | 23 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGEGRGSLLTCGDVEENPGPRMALPVTALLLPLALLLHAAR |
| | | PEVQLQQSGAELVKPGASVKMSCKASGYTFTSYNMHWVKQTPGQGLEWIG |
| | | AIYPGNGDTSYNQKFKGKATLTADKSSSTAYMQLSSLTSEDSADYYCARSNY |
| | | YGSSYWFFDVWGAGTTVTVSSGSTSGGGSGGGSGGGGSSDIVLTQSPAILSAS |
| | | PGEKVTMTCRASSSVNYMDWYQKKPGSSPKPWIYATSNLASGVPARFSGSGS |
| | | GTSYSLTISRVEAEDAATYYCQQWSFNPPTFGGGTKLEIKPSGGGGSGGGGSG |
| | | GGGSGGGGSGGGGSPYEWSNSSDPLLVSVTGNPSNSWPSPTEPSSKTGNPRH |
| | | LHVLIGTSVVKIPFTILLFFLLHRWCSNKKNAAVMDQEPAGNRTVNSEDSDEQ |
| | | DHQEVSYAVEGGGATNFSLLKQAGDVEENPGPRMALPVTALLLPLALLLHAA |
| | | RPGSQSALTQTASVSGSLGQKVTISCTGSRSNIGANYVGWYQQVPGIGPRTVIF |
| | | GYNYRPSGVPDRFSSSKSGNSATLTISGLQAEDEAEYYCSTWDSSLNAYVFGS |
| | | GTQLTVLGGGSSRSSSSGGGGSGGGGEVPLVESGGDLVKPGGSLRLSCVASGF |
| | | TLSNYGMSWVRQSPGKGLQWVAAIRSGGNTYYADAVKGRFTISRDNAKNTL |
| | | YLQMNSLRAEDTAVYYCASQWGDWGHFEYWGQGTLVTVSSASEQKLISEED |
| | | LPSGGGGSGGGGSSPTEPSSKTGNPRHLHVLIGTSVVKIPFTILLFFLLHRWCS |
| | | NKKNAAVMDQEPAGNRTVNSEDSDEQDHQEVSYA |
| 723 | 24 | MGGLEPCSRFLLLPLLLAVSGLRPVQVQAQSDCSCSTVSPGVLAGIVMGDLV |
| | AA | LTVLIALAVYFLGRLVPRGRGAAEAATRKQRITETESPYQELQGQRSDVYSDL |
| | | NTQRPYYKVEGGGATNFSLLKQAGDVEENPGPRMALPVTALLLPLALLLHA |
| | | ARPEVQLQQSGAELVKPGASVKMSCKASGYTFTSYNMHWVKQTPGQGLEW |
| | | IGAIYPGNGDTSYNQKFKGKATLTADKSSSTAYMQLSSLTSEDSADYYCARSN |
| | | YYGSSYWFFDVWGAGTTVTVSSGSTSGGGSGGGSGGGGSSDIVLTQSPAILS |
| | | ASPGEKVTMTCRASSSVNYMDWYQKKPGSSPKPWIYATSNLASGVPARFSGS |
| | | GSGTSYSLTISRVEAEDAATYYCQQWSFNPPTFGGGTKLEIKPSGGGGSGGGG |
| | | SGGGGSGGGGSGGGGSPYEWSNSSDPLLVSVTGNPSNSWPSPTEPSSKTGNP |
| | | RHLHVLIGTSVVKIPFTILLFFLLHRWCSNKKNAAVMDQEPAGNRTVNSEDSD |
| | | EQDHQEVSYAVEGGGEGRGSLLTCGDVEENPGPRMALPVTALLLPLALLLHA |
| | | ARPGSQSALTQTASVSGSLGQKVTISCTGSRSNIGANYVGWYQQVPGIGPRTV |
| | | IFGYNYRPSGVPDRFSSSKSGNSATLTISGLQAEDEAEYYCSTWDSSLNAYVF |
| | | GSGTQLTVLGGGSSRSSSSGGGGSGGGGEVPLVESGGDLVKPGGSLRLSCVAS |
| | | GFTLSNYGMSWVRQSPGKGLQWVAAIRSGGNTYYADAVKGRFTISRDNAKN |
| | | TLYLQMNSLRAEDTAVYYCASQWGDWGHFEYWGQGTLVTVSSASEQKLISE |
| | | EDLPSGGGGSGGGGSSPTEPSSKTGNPRHLHVLIGTSVVKIPFTILLFFLLHRW |
| | | CSNKKNAAVMDQEPAGNRTVNSEDSDEQDHQEVSYA |
| 724 | 9 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGGATCCCAGTCTGCGCTGACTCAAACG |
| | | GCCTCAGTGTCTGGGTCCCTGGGCCAGAAGGTCACCATCTCCTGCACTGG |
| | | AAGCAGGTCCAACATCGGTGCAAATTATGTGGGCTGGTATCAACAGGTCCC |
| | | AGGAATAGGCCCTAGAACCGTCATTTTTGGTTATAATTATCGACCTTCAGGG |
| | | GTCCCCGATCGATTCTCTAGCTCCAAGTCAGGCAATTCAGCCACCCTGACC |
| | | ATCTCTGGGCTCCAGGCTGAGGACGAGGCTGAGTATTATTGTTCAACATGG |
| | | GACAGCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCTCGGG |
| | | CGGTGGTGGGGAGGTGCCACTGGTGGAATCTGGGGGAGACCTGGTGAAG |
| | | CCTGGGGGGTCCTTGAGACTGTCCTGTGTGGCCTCTGGATTCACCCTCAGT |
| | | AACTATGGCATGAGCTGGGTCCGTCAGTCTCCAGGGAAGGGGCTGCAGTG |
| | | GGTCGCAGCTATTAGGAGTGGTGGAAACACATACTACGCAGACGCTGTGA |
| | | AGGGCCGATTCACCATCTCCAGAGACAACGCCAAGAACACGCTGTATCTG |
| | | CAGATGAACAGCCTGAGAGCCGAGGACACGGCCGTGTATTACTGTGCGTC |
| | | CCAATGGGGTGATTGGGGCCACTTTGAGTACTGGGGCCAGGGCACCCTGG |
| | | TCACCGTCTCCTCAGCTAGCGAGCAGAAATTAATCTCAGAGGAGGACCTG |
| | | CCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCT |
| | | CAGTGGTCAAAATCCCTTTCACCATCCTCCTCTTCTTTCTCCTTCATCGCTG |
| | | GTGCTCCAACAAAAAAAATGCTGCTGTAATGGACCAAGAGCCTGCAGGGA |
| | | ACAGAACAGTGAACAGCGAGGATTCTGATGAACAAGACCATCAGGAGGT |
| | | GTCATACGCATAA |
| 725 | 10 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGAGGTGCAGCTGCAGCAGTCTGGGGCT |
| | | GAGCTGGTGAAGCCTGGGGCCTCAGTGAAGATGTCCTGCAAGGCTTCTGG |
| | | CTACACATTTACCAGTTACAATATGCACTGGGTAAAGCAGACACCTGGACA |
| | | GGGCCTGGAATGGATTGGAGCTATTTATCCAGGAAATGGTGATACTTCCTAC |
| | | AATCAGAAGTTCAAAGGCAAGGCCACATTGACTGCAGACAAATCCTCCAG |
| | | CACAGCCTACATGCAGCTCAGCAGCCTGACATCTGAGGACTCTGCGGACT |
| | | ATTACTGTGCAAGATCTAATTATTACGGTAGTAGCTACTGGTTCTTCGATGTC |
| | | TGGGGCGCAGGGACCACGGTCACCGTCTCCTCAGGCAGTACTAGCGGTGG |
| | | TGGCTCCGGGGGCGGTTCCGGTGGGGGCGGCAGCAGCGACATTGTGCTGA |
| | | CCCAATCTCCAGCTATCCTGTCTGCATCTCCAGGGGAGAAGGTCACAATGA |
| | | CTTGCAGGGCCAGCTCAAGTGTAAATTACATGGACTGGTACCAGAAGAAG |
| | | CCAGGTTCCTCCCCCAAACCCTGGATTTATGCCACATCCAACCTGGCTTCT |
| | | GGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCA |
| | | GTGGAGTTTTAATCCACCCACGTTCGGAGGGGGGACCAAGCTGGAAATAA |
| | | AAGGAGCTAGCCACCATCACCATCACCATCCTAGCGGTGGCGGAGGTTCT |
| | | GGAGGTGGGGGTTCCTCACCCACTGAACCAAGCTCCAAAACCGGTAACCC |
| | | CAGACACCTGCATGTTCTGATTGGGACCTCAGTGGTCAAAATCCCTTTCAC |
| | | CATCCTCCTCTTCTTTCTCCTTCATCGCTGGTGCTCCAACAAAAAAAATGCT |
| | | GCTGTAATGGACCAAGAGCCTGCAGGGAACAGAACAGTGAACAGCGAGG |
| | | ATTCTGATGAACAAGACCATCAGGAGGTGTCATACGCATAA |
| 726 | 11 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCACTCCCCGTCACCGCGCTCCTCTTGCCCCTCGCGC |
| | | TGCTCCTTCATGCTGCGAGGCCGGAGGTGCAGCTGCAGCAGTCTGGGGCT |
| | | GAGCTGGTGAAGCCTGGGGCCTCAGTGAAGATGTCCTGCAAGGCTTCTGG |
| | | CTACACATTTACCAGTTACAATATGCACTGGGTAAAGCAGACACCTGGACA |
| | | GGGCCTGGAATGGATTGGAGCTATTTATCCAGGAAATGGTGATACTTCCTAC |
| | | AATCAGAAGTTCAAAGGCAAGGCCACATTGACTGCAGACAAATCCTCCAG |
| | | CACAGCCTACATGCAGCTCAGCAGCCTGACATCTGAGGACTCTGCGGACT |
| | | ATTACTGTGCAAGATCTAATTATTACGGTAGTAGCTACTGGTTCTTCGATGTC |
| | | TGGGGCGCAGGGACCACGGTCACCGTCTCCTCAGGCAGTACTAGCGGTGG |
| | | TGGCTCCGGGGGCGGTTCCGGTGGGGGCGGCAGCAGCGACATTGTGCTGA |
| | | CCCAATCTCCAGCTATCCTGTCTGCATCTCCAGGGGAGAAGGTCACAATGA |
| | | CTTGCAGGGCCAGCTCAAGTGTAAATTACATGGACTGGTACCAGAAGAAG |
| | | CCAGGTTCCTCCCCCAAACCCTGGATTTATGCCACATCCAACCTGGCTTCT |
| | | GGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCA |
| | | GTGGAGTTTTAATCCACCCACGTTCGGAGGGGGGACCAAGCTGGAAATAA |
| | | AACCTAGCGGAGGCGGCGGCAGCGGCGGGGGAGGGTCCGGAGGGGGTGG |
| | | TTCTGGTGGAGGAGGATCGGGAGGCGGTGGCAGCTCACCAACAGAGCCG |
| | | TCAAGTAAAACCGGGAACCCGAGACATCTCCACGTTCTGATCGGAACATC |
| | | CGTCGTAAAAATCCCTTTCACCATATTGTTGTTTTTCTTGTTGCACCGATGG |
| | | TGCTCTAACAAGAAGAACGCAGCCGTAATGGACCAAGAGCCAGCCGGAA |
| | | ACAGGACTGTCAATTCCGAAGATTCCGATGAGCAGGATCACCAAGAAGTT |
| | | AGCTACGCATAA |
| 727 | 12 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCACTCCCCGTCACCGCGCTCCTCTTGCCCCTCGCGC |
| | | TGCTCCTTCATGCTGCGAGGCCGGAGGTGCAGCTGCAGCAGTCTGGGGCT |
| | | GAGCTGGTGAAGCCTGGGGCCTCAGTGAAGATGTCCTGCAAGGCTTCTGG |
| | | CTACACATTTACCAGTTACAATATGCACTGGGTAAAGCAGACACCTGGACA |
| | | GGGCCTGGAATGGATTGGAGCTATTTATCCAGGAAATGGTGATACTTCCTAC |
| | | AATCAGAAGTTCAAAGGCAAGGCCACATTGACTGCAGACAAATCCTCCAG |
| | | CACAGCCTACATGCAGCTCAGCAGCCTGACATCTGAGGACTCTGCGGACT |
| | | ATTACTGTGCAAGATCTAATTATTACGGTAGTAGCTACTGGTTCTTCGATGTC |
| | | TGGGGCGCAGGGACCACGGTCACCGTCTCCTCAGGCAGTACTAGCGGTGG |
| | | TGGCTCCGGGGGCGGTTCCGGTGGGGGCGGCAGCAGCGACATTGTGCTGA |
| | | CCCAATCTCCAGCTATCCTGTCTGCATCTCCAGGGGAGAAGGTCACAATGA |
| | | CTTGCAGGGCCAGCTCAAGTGTAAATTACATGGACTGGTACCAGAAGAAG |
| | | CCAGGTTCCTCCCCCAAACCCTGGATTTATGCCACATCCAACCTGGCTTCT |
| | | GGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCA |
| | | GTGGAGTTTTAATCCACCCACGTTCGGAGGGGGGACCAAGCTGGAAATAA |
| | | AACCTAGCGGAGGCGGCGGCAGCGGCGGGGGAGGGTCCGGAGGGGGTGG |
| | | TTCTGGTGGAGGAGGATCGGGAGGCGGTGGCAGCCCCTATGAGTGGTCAA |
| | | ACTCGAGTGACCCACTGCTTGTTTCTGTCACAGGAAACCCTTCAAATAGTT |
| | | GGCCTTCACCAACAGAGCCGTCAAGTAAAACCGGGAACCCGAGACATCTC |
| | | CACGTTCTGATCGGAACATCCGTCGTAAAAATCCCTTTCACCATATTGTTGT |
| | | TTTTCTTGTTGCACCGATGGTGCTCTAACAAGAAGAACGCAGCCGTAATGG |
| | | ACCAAGAGCCAGCCGGAAACAGGACTGTCAATTCCGAAGATTCCGATGAG |
| | | CAGGATCACCAAGAAGTTAGCTACGCATAA |
| 728 | 13 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGGATCCCAGTCTGCGCTGACTCAAACG |
| | | GCCTCAGTGTCTGGGTCCCTGGGCCAGAAGGTCACCATCTCCTGCACTGG |
| | | AAGCAGGTCCAACATCGGTGCAAATTATGTGGGCTGGTATCAACAGGTCCC |
| | | AGGAATAGGCCCTAGAACCGTCATTTTTGGTTATAATTATCGACCTTCAGGG |
| | | GTCCCCGATCGATTCTCTAGCTCCAAGTCAGGCAATTCAGCCACCCTGACC |
| | | ATCTCTGGGCTCCAGGCTGAGGACGAGGCTGAGTATTATTGTTCAACATGG |
| | | GACAGCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCTCGGG |
| | | CGGTGGTGGGGAGGTGCCACTGGTGGAATCTGGGGGAGACCTGGTGAAG |
| | | CCTGGGGGGTCCTTGAGACTGTCCTGTGTGGCCTCTGGATTCACCCTCAGT |
| | | AACTATGGCATGAGCTGGGTCCGTCAGTCTCCAGGGAAGGGGCTGCAGTG |
| | | GGTCGCAGCTATTAGGAGTGGTGGAAACACATACTACGCAGACGCTGTGA |
| | | AGGGCCGATTCACCATCTCCAGAGACAACGCCAAGAACACGCTGTATCTG |
| | | CAGATGAACAGCCTGAGAGCCGAGGACACGGCCGTGTATTACTGTGCGTC |
| | | CCAATGGGGTGATTGGGGCCACTTTGAGTACTGGGGCCAGGGCACCCTGG |
| | | TCACCGTCTCCTCAGCTAGCGAGCAGAAATTAATCTCAGAGGAGGACCTG |
| | | CCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCT |
| | | CAGTGGTCAAAATCCCTTTCACCATCCTCCTCTTCTTTCTCCTTCATCGCTG |
| | | GTGCTCCAACAAAAAAAATGCTGCTGTAATGGACCAAGAGCCTGCAGGGA |
| | | ACAGAACAGTGAACAGCGAGGATTCTGATGAACAAGACCATCAGGAGGT |
| | | GTCATACGCAGTCGAGGGCGGCGGAGCCACGAACTTCTCTCTGTTAAAGC |
| | | AAGCAGGAGACGTGGAAGAAAACCCCGGTCCTAGGATGGCCTTACCAGTG |
| | | ACCGCCTTGCTCCTGCCGCTGGCCTTGCTGCTCCACGCCGCCAGGCCGGA |
| | | GGTGCAGCTGCAGCAGTCTGGGGCTGAGCTGGTGAAGCCTGGGGCCTCAG |
| | | TGAAGATGTCCTGCAAGGCTTCTGGCTACACATTTACCAGTTACAATATGC |
| | | ACTGGGTAAAGCAGACACCTGGACAGGGCCTGGAATGGATTGGAGCTATT |
| | | TATCCAGGAAATGGTGATACTTCCTACAATCAGAAGTTCAAAGGCAAGGCC |
| | | ACATTGACTGCAGACAAATCCTCCAGCACAGCCTACATGCAGCTCAGCAG |
| | | CCTGACATCTGAGGACTCTGCGGACTATTACTGTGCAAGATCTAATTATTAC |
| | | GGTAGTAGCTACTGGTTCTTCGATGTCTGGGGCGCAGGGACCACGGTCACC |
| | | GTCTCCTCAGGCAGTACTAGCGGTGGTGGCTCCGGGGGCGGTTCCGGTGG |
| | | GGGCGGCAGCAGCGACATTGTGCTGACCCAATCTCCAGCTATCCTGTCTGC |
| | | ATCTCCAGGGGAGAAGGTCACAATGACTTGCAGGGCCAGCTCAAGTGTAA |
| | | ATTACATGGACTGGTACCAGAAGAAGCCAGGTTCCTCCCCCAAACCCTGG |
| | | ATTTATGCCACATCCAACCTGGCTTCTGGAGTCCCTGCTCGCTTCAGTGGC |
| | | AGTGGGTCTGGGACCTCTTACTCTCTCACAATCAGCAGAGTGGAGGCTGA |
| | | AGATGCTGCCACTTATTACTGCCAGCAGTGGAGTTTTAATCCACCCACGTT |
| | | CGGAGGGGGGACCAAGCTGGAAATAAAAGGAGCTAGCCACCATCACCATC |
| | | ACCATCCTAGCGGTGGCGGAGGTTCTGGAGGTGGGGGTTCCTCACCCACT |
| | | GAACCAAGCTCCAAAACCGGTAACCCCAGACACCTGCATGTTCTGATTGG |
| | | GACCTCAGTGGTCAAAATCCCTTTCACCATCCTCCTCTTCTTTCTCCTTCAT |
| | | CGCTGGTGCTCCAACAAAAAAAATGCTGCTGTAATGGACCAAGAGCCTGC |
| | | AGGGAACAGAACAGTGAACAGCGAGGATTCTGATGAACAAGACCATCAG |
| | | GAGGTGTCATACGCATAATGAGC |
| 729 | 14 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGAGGTGCAGCTGCAGCAGTCTGGGGCT |
| | | GAGCTGGTGAAGCCTGGGGCCTCAGTGAAGATGTCCTGCAAGGCTTCTGG |
| | | CTACACATTTACCAGTTACAATATGCACTGGGTAAAGCAGACACCTGGACA |
| | | GGGCCTGGAATGGATTGGAGCTATTTATCCAGGAAATGGTGATACTTCCTAC |
| | | AATCAGAAGTTCAAAGGCAAGGCCACATTGACTGCAGACAAATCCTCCAG |
| | | CACAGCCTACATGCAGCTCAGCAGCCTGACATCTGAGGACTCTGCGGACT |
| | | ATTACTGTGCAAGATCTAATTATTACGGTAGTAGCTACTGGTTCTTCGATGTC |
| | | TGGGGCGCAGGGACCACGGTCACCGTCTCCTCAGGCAGTACTAGCGGTGG |
| | | TGGCTCCGGGGGCGGTTCCGGTGGGGGCGGCAGCAGCGACATTGTGCTGA |
| | | CCCAATCTCCAGCTATCCTGTCTGCATCTCCAGGGGAGAAGGTCACAATGA |
| | | CTTGCAGGGCCAGCTCAAGTGTAAATTACATGGACTGGTACCAGAAGAAG |
| | | CCAGGTTCCTCCCCCAAACCCTGGATTTATGCCACATCCAACCTGGCTTCT |
| | | GGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCA |
| | | GTGGAGTTTTAATCCACCCACGTTCGGAGGGGGGACCAAGCTGGAAATAA |
| | | AAGGAGCTAGCCACCATCACCATCACCATCCTAGCGGTGGCGGAGGTTCT |
| | | GGAGGTGGGGGTTCCTCACCCACTGAACCAAGCTCCAAAACCGGTAACCC |
| | | CAGACACCTGCATGTTCTGATTGGGACCTCAGTGGTCAAAATCCCTTTCAC |
| | | CATCCTCCTCTTCTTTCTCCTTCATCGCTGGTGCTCCAACAAAAAAAATGCT |
| | | GCTGTAATGGACCAAGAGCCTGCAGGGAACAGAACAGTGAACAGCGAGG |
| | | ATTCTGATGAACAAGACCATCAGGAGGTGTCATACGCAGTCGAGGGCGGC |
| | | GGAGCCACGAACTTCTCTCTGTTAAAGCAAGCAGGAGACGTGGAAGAAA |
| | | ACCCCGGTCCTAGGATGGCCTTACCAGTGACCGCCTTGCTCCTGCCGCTGG |
| | | CCTTGCTGCTCCACGCCGCCAGGCCGGGATCCCAGTCTGCGCTGACTCAA |
| | | ACGGCCTCAGTGTCTGGGTCCCTGGGCCAGAAGGTCACCATCTCCTGCAC |
| | | TGGAAGCAGGTCCAACATCGGTGCAAATTATGTGGGCTGGTATCAACAGGT |
| | | CCCAGGAATAGGCCCTAGAACCGTCATTTTTGGTTATAATTATCGACCTTCA |
| | | GGGGTCCCCGATCGATTCTCTAGCTCCAAGTCAGGCAATTCAGCCACCCTG |
| | | ACCATCTCTGGGCTCCAGGCTGAGGACGAGGCTGAGTATTATTGTTCAACA |
| | | TGGGACAGCAGTCTCAACGCTTACGTGTTCGGCTCAGGGACCCAGCTGAC |
| | | CGTCCTCGGCGGTGGTTCCTCTAGATCTTCCTCCTCTGGTGGCGGTGGCTC |
| | | GGGCGGTGGTGGGGAGGTGCCACTGGTGGAATCTGGGGGAGACCTGGTG |
| | | AAGCCTGGGGGGTCCTTGAGACTGTCCTGTGTGGCCTCTGGATTCACCCTC |
| | | AGTAACTATGGCATGAGCTGGGTCCGTCAGTCTCCAGGGAAGGGGCTGCA |
| | | GTGGGTCGCAGCTATTAGGAGTGGTGGAAACACATACTACGCAGACGCTG |
| | | TGAAGGGCCGATTCACCATCTCCAGAGACAACGCCAAGAACACGCTGTAT |
| | | CTGCAGATGAACAGCCTGAGAGCCGAGGACACGGCCGTGTATTACTGTGC |
| | | GTCCCAATGGGGTGATTGGGGCCACTTTGAGTACTGGGGCCAGGGCACCC |
| | | TGGTCACCGTCTCCTCAGCTAGCGAGCAGAAATTAATCTCAGAGGAGGAC |
| | | CTGCCTAGCGGTGGCGGAGGTTCTGGAGGTGGGGGTTCCTCACCCACTGA |
| | | ACCAAGCTCCAAAACCGGTAACCCCAGACACCTGCATGTTCTGATTGGGA |
| | | CCTCAGTGGTCAAAATCCCTTTCACCATCCTCCTCTTCTTTCTCCTTCATCG |
| | | CTGGTGCTCCAACAAAAAAAATGCTGCTGTAATGGACCAAGAGCCTGCAG |
| | | GGAACAGAACAGTGAACAGCGAGGATTCTGATGAACAAGACCATCAGGA |
| | | GGTGTCATACGCATAATGAGC |
| 730 | 15 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGCCACGAACTTCTCTCTGTTAAAGCAAGCAGGAGACGTGGAAGAAA |
| | | ACCCCGGTCCTAGGATGGCCTTACCAGTGACCGCCTTGCTCCTGCCGCTGG |
| | | CCTTGCTGCTCCACGCCGCCAGGCCGGAGGTGCAGCTGCAGCAGTCTGGG |
| | | GCTGAGCTGGTGAAGCCTGGGGCCTCAGTGAAGATGTCCTGCAAGGCTTC |
| | | TGGCTACACATTTACCAGTTACAATATGCACTGGGTAAAGCAGACACCTGG |
| | | ACAGGGCCTGGAATGGATTGGAGCTATTTATCCAGGAAATGGTGATACTTC |
| | | CTACAATCAGAAGTTCAAAGGCAAGGCCACATTGACTGCAGACAAATCCT |
| | | CCAGCACAGCCTACATGCAGCTCAGCAGCCTGACATCTGAGGACTCTGCG |
| | | GACTATTACTGTGCAAGATCTAATTATTACGGTAGTAGCTACTGGTTCTTCG |
| | | ATGTCTGGGGCGCAGGGACCACGGTCACCGTCTCCTCAGGCAGTACTAGC |
| | | GGTGGTGGCTCCGGGGGCGGTTCCGGTGGGGGCGGCAGCAGCGACATTGT |
| | | GCTGACCCAATCTCCAGCTATCCTGTCTGCATCTCCAGGGGAGAAGGTCAC |
| | | AATGACTTGCAGGGCCAGCTCAAGTGTAAATTACATGGACTGGTACCAGA |
| | | AGAAGCCAGGTTCCTCCCCCAAACCCTGGATTTATGCCACATCCAACCTGG |
| | | CTTCTGGAGTCCCTGCTCGCTTCAGTGGCAGTGGGTCTGGGACCTCTTACT |
| | | CTCTCACAATCAGCAGAGTGGAGGCTGAAGATGCTGCCACTTATTACTGCC |
| | | AGCAGTGGAGTTTTAATCCACCCACGTTCGGAGGGGGGACCAAGCTGGAA |
| | | ATAAAAGGAGCTAGCCACCATCACCATCACCATCCTAGCGGTGGCGGAGGT |
| | | TCTGGAGGTGGGGGTTCCTCACCCACTGAACCAAGCTCCAAAACCGGTAA |
| | | CCCCAGACACCTGCATGTTCTGATTGGGACCTCAGTGGTCAAAATCCCTTT |
| | | CACCATCCTCCTCTTCTTTCTCCTTCATCGCTGGTGCTCCAACAAAAAAAAT |
| | | GCTGCTGTAATGGACCAAGAGCCTGCAGGGAACAGAACAGTGAACAGCG |
| | | AGGATTCTGATGAACAAGACCATCAGGAGGTGTCATACGCAGTCGAGGGC |
| | | GGCGGAGCCACGAACTTCTCTCTGTTAAAGCAAGCAGGAGACGTGGAAG |
| | | AAAACCCCGGTCCTAGGATGGCCTTACCAGTGACCGCCTTGCTCCTGCCGC |
| | | TGGCCTTGCTGCTCCACGCCGCCAGGCCGGGATCCCAGTCTGCGCTGACT |
| | | CAAACGGCCTCAGTGTCTGGGTCCCTGGGCCAGAAGGTCACCATCTCCTG |
| | | CACTGGAAGCAGGTCCAACATCGGTGCAAATTATGTGGGCTGGTATCAACA |
| | | GGTCCCAGGAATAGGCCCTAGAACCGTCATTTTTGGTTATAATTATCGACCT |
| | | TCAGGGGTCCCCGATCGATTCTCTAGCTCCAAGTCAGGCAATTCAGCCACC |
| | | CTGACCATCTCTGGGCTCCAGGCTGAGGACGAGGCTGAGTATTATTGTTCA |
| | | ACATGGGACAGCAGTCTCAACGCTTACGTGTTCGGCTCAGGGACCCAGCT |
| | | GACCGTCCTCGGCGGTGGTTCCTCTAGATCTTCCTCCTCTGGTGGCGGTGG |
| | | CTCGGGCGGTGGTGGGGAGGTGCCACTGGTGGAATCTGGGGGAGACCTG |
| | | GTGAAGCCTGGGGGGTCCTTGAGACTGTCCTGTGTGGCCTCTGGATTCACC |
| | | CTCAGTAACTATGGCATGAGCTGGGTCCGTCAGTCTCCAGGGAAGGGGCT |
| | | GCAGTGGGTCGCAGCTATTAGGAGTGGTGGAAACACATACTACGCAGACG |
| | | CTGTGAAGGGCCGATTCACCATCTCCAGAGACAACGCCAAGAACACGCTG |
| | | TATCTGCAGATGAACAGCCTGAGAGCCGAGGACACGGCCGTGTATTACTGT |
| | | GCGTCCCAATGGGGTGATTGGGGCCACTTTGAGTACTGGGGCCAGGGCAC |
| | | CCTGGTCACCGTCTCCTCAGCTAGCGAGCAGAAATTAATCTCAGAGGAGG |
| | | ACCTGCCTAGCGGTGGCGGAGGTTCTGGAGGTGGGGGTTCCTCACCCACT |
| | | GAACCAAGCTCCAAAACCGGTAACCCCAGACACCTGCATGTTCTGATTGG |
| | | GACCTCAGTGGTCAAAATCCCTTTCACCATCCTCCTCTTCTTTCTCCTTCAT |
| | | CGCTGGTGCTCCAACAAAAAAAATGCTGCTGTAATGGACCAAGAGCCTGC |
| | | AGGGAACAGAACAGTGAACAGCGAGGATTCTGATGAACAAGACCATCAG |
| | | GAGGTGTCATACGCATAATGAGC |
| 730 | 16 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGCCACGAACTTCTCTCTGTTAAAGCAAGCAGGAGACGTGGAAGAAA |
| | | ACCCCGGTCCTAGGATGGCCTTACCAGTGACCGCCTTGCTCCTGCCGCTGG |
| | | CCTTGCTGCTCCACGCCGCCAGGCCGGGATCCCAGTCTGCGCTGACTCAA |
| | | ACGGCCTCAGTGTCTGGGTCCCTGGGCCAGAAGGTCACCATCTCCTGCAC |
| | | TGGAAGCAGGTCCAACATCGGTGCAAATTATGTGGGCTGGTATCAACAGGT |
| | | CCCAGGAATAGGCCCTAGAACCGTCATTTTTGGTTATAATTATCGACCTTCA |
| | | GGGGTCCCCGATCGATTCTCTAGCTCCAAGTCAGGCAATTCAGCCACCCTG |
| | | ACCATCTCTGGGCTCCAGGCTGAGGACGAGGCTGAGTATTATTGTTCAACA |
| | | TGGGACAGCAGTCTCAACGCTTACGTGTTCGGCTCAGGGACCCAGCTGAC |
| | | CGTCCTCGGCGGTGGTTCCTCTAGATCTTCCTCCTCTGGTGGCGGTGGCTC |
| | | GGGCGGTGGTGGGGAGGTGCCACTGGTGGAATCTGGGGGAGACCTGGTG |
| | | AAGCCTGGGGGGTCCTTGAGACTGTCCTGTGTGGCCTCTGGATTCACCCTC |
| | | AGTAACTATGGCATGAGCTGGGTCCGTCAGTCTCCAGGGAAGGGGCTGCA |
| | | GTGGGTCGCAGCTATTAGGAGTGGTGGAAACACATACTACGCAGACGCTG |
| | | TGAAGGGCCGATTCACCATCTCCAGAGACAACGCCAAGAACACGCTGTAT |
| | | CTGCAGATGAACAGCCTGAGAGCCGAGGACACGGCCGTGTATTACTGTGC |
| | | GTCCCAATGGGGTGATTGGGGCCACTTTGAGTACTGGGGCCAGGGCACCC |
| | | TGGTCACCGTCTCCTCAGCTAGCGAGCAGAAATTAATCTCAGAGGAGGAC |
| | | CTGCCTAGCGGTGGCGGAGGTTCTGGAGGTGGGGGTTCCTCACCCACTGA |
| | | ACCAAGCTCCAAAACCGGTAACCCCAGACACCTGCATGTTCTGATTGGGA |
| | | CCTCAGTGGTCAAAATCCCTTTCACCATCCTCCTCTTCTTTCTCCTTCATCG |
| | | CTGGTGCTCCAACAAAAAAAATGCTGCTGTAATGGACCAAGAGCCTGCAG |
| | | GGAACAGAACAGTGAACAGCGAGGATTCTGATGAACAAGACCATCAGGA |
| | | GGTGTCATACGCAGTCGAGGGCGGCGGAGCCACGAACTTCTCTCTGTTAA |
| | | AGCAAGCAGGAGACGTGGAAGAAAACCCCGGTCCTAGGATGGCCTTACC |
| | | AGTGACCGCCTTGCTCCTGCCGCTGGCCTTGCTGCTCCACGCCGCCAGGC |
| | | CGGAGGTGCAGCTGCAGCAGTCTGGGGCTGAGCTGGTGAAGCCTGGGGC |
| | | CTCAGTGAAGATGTCCTGCAAGGCTTCTGGCTACACATTTACCAGTTACAA |
| | | TATGCACTGGGTAAAGCAGACACCTGGACAGGGCCTGGAATGGATTGGAG |
| | | CTATTTATCCAGGAAATGGTGATACTTCCTACAATCAGAAGTTCAAAGGCA |
| | | AGGCCACATTGACTGCAGACAAATCCTCCAGCACAGCCTACATGCAGCTC |
| | | AGCAGCCTGACATCTGAGGACTCTGCGGACTATTACTGTGCAAGATCTAAT |
| | | TATTACGGTAGTAGCTACTGGTTCTTCGATGTCTGGGGCGCAGGGACCACG |
| | | GTCACCGTCTCCTCAGGCAGTACTAGCGGTGGTGGCTCCGGGGGCGGTTC |
| | | CGGTGGGGGCGGCAGCAGCGACATTGTGCTGACCCAATCTCCAGCTATCCT |
| | | GTCTGCATCTCCAGGGGAGAAGGTCACAATGACTTGCAGGGCCAGCTCAA |
| | | GTGTAAATTACATGGACTGGTACCAGAAGAAGCCAGGTTCCTCCCCCAAA |
| | | CCCTGGATTTATGCCACATCCAACCTGGCTTCTGGAGTCCCTGCTCGCTTCA |
| | | GTGGCAGTGGGTCTGGGACCTCTTACTCTCTCACAATCAGCAGAGTGGAG |
| | | GCTGAAGATGCTGCCACTTATTACTGCCAGCAGTGGAGTTTTAATCCACCC |
| | | ACGTTCGGAGGGGGGACCAAGCTGGAAATAAAAGGAGCTAGCCACCATC |
| | | ACCATCACCATCCTAGCGGTGGCGGAGGTTCTGGAGGTGGGGGTTCCTCA |
| | | CCCACTGAACCAAGCTCCAAAACCGGTAACCCCAGACACCTGCATGTTCT |
| | | GATTGGGACCTCAGTGGTCAAAATCCCTTTCACCATCCTCCTCTTCTTTCTC |
| | | CTTCATCGCTGGTGCTCCAACAAAAAAAATGCTGCTGTAATGGACCAAGA |
| | | GCCTGCAGGGAACAGAACAGTGAACAGCGAGGATTCTGATGAACAAGAC |
| | | CATCAGGAGGTGTCATACGCATAATGAGC |
| 732 | 17 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGAGGTGCAGCTGCAGCAGTCTGGGGCT |
| | | GAGCTGGTGAAGCCTGGGGCCTCAGTGAAGATGTCCTGCAAGGCTTCTGG |
| | | CTACACATTTACCAGTTACAATATGCACTGGGTAAAGCAGACACCTGGACA |
| | | GGGCCTGGAATGGATTGGAGCTATTTATCCAGGAAATGGTGATACTTCCTAC |
| | | AATCAGAAGTTCAAAGGCAAGGCCACATTGACTGCAGACAAATCCTCCAG |
| | | CACAGCCTACATGCAGCTCAGCAGCCTGACATCTGAGGACTCTGCGGACT |
| | | ATTACTGTGCAAGATCTAATTATTACGGTAGTAGCTACTGGTTCTTCGATGTC |
| | | TGGGGCGCAGGGACCACGGTCACCGTCTCCTCAGGCAGTACTAGCGGTGG |
| | | TGGCTCCGGGGGCGGTTCCGGTGGGGGCGGCAGCAGCGACATTGTGCTGA |
| | | CCCAATCTCCAGCTATCCTGTCTGCATCTCCAGGGGAGAAGGTCACAATGA |
| | | CTTGCAGGGCCAGCTCAAGTGTAAATTACATGGACTGGTACCAGAAGAAG |
| | | CCAGGTTCCTCCCCCAAACCCTGGATTTATGCCACATCCAACCTGGCTTCT |
| | | GGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCA |
| | | GTGGAGTTTTAATCCACCCACGTTCGGAGGGGGGACCAAGCTGGAAATAA |
| | | AAGGAGCTAGCCACCATCACCATCACCATCCTAGCGGTGGCGGAGGTTCT |
| | | GGAGGTGGGGGTTCCTCACCCACTGAACCAAGCTCCAAAACCGGTAACCC |
| | | CAGACACCTGCATGTTCTGATTGGGACCTCAGTGGTCAAAATCCCTTTCAC |
| | | CATCCTCCTCTTCTTTCTCCTTCATCGCTGGTGCTCCAACAAAAAAAATGCT |
| | | GCTGTAATGGACCAAGAGCCTGCAGGGAACAGAACAGTGAACAGCGAGG |
| | | ATTCTGATGAACAAGACCATCAGGAGGTGTCATACGCAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGGATCCCAGTCTGCGCTGACTCAAACG |
| | | GCCTCAGTGTCTGGGTCCCTGGGCCAGAAGGTCACCATCTCCTGCACTGG |
| | | AAGCAGGTCCAACATCGGTGCAAATTATGTGGGCTGGTATCAACAGGTCCC |
| | | AGGAATAGGCCCTAGAACCGTCATTTTTGGTTATAATTATCGACCTTCAGGG |
| | | GTCCCCGATCGATTCTCTAGCTCCAAGTCAGGCAATTCAGCCACCCTGACC |
| | | ATCTCTGGGCTCCAGGCTGAGGACGAGGCTGAGTATTATTGTTCAACATGG |
| | | GACAGCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCTCGGG |
| | | CGGTGGTGGGGAGGTGCCACTGGTGGAATCTGGGGGAGACCTGGTGAAG |
| | | CCTGGGGGGTCCTTGAGACTGTCCTGTGTGGCCTCTGGATTCACCCTCAGT |
| | | AACTATGGCATGAGCTGGGTCCGTCAGTCTCCAGGGAAGGGGCTGCAGTG |
| | | GGTCGCAGCTATTAGGAGTGGTGGAAACACATACTACGCAGACGCTGTGA |
| | | AGGGCCGATTCACCATCTCCAGAGACAACGCCAAGAACACGCTGTATCTG |
| | | CAGATGAACAGCCTGAGAGCCGAGGACACGGCCGTGTATTACTGTGCGTC |
| | | CCAATGGGGTGATTGGGGCCACTTTGAGTACTGGGGCCAGGGCACCCTGG |
| | | TCACCGTCTCCTCAGCTAGCGAGCAGAAATTAATCTCAGAGGAGGACCTG |
| | | CCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCT |
| | | CAGTGGTCAAAATCCCTTTCACCATCCTCCTCTTCTTTCTCCTTCATCGCTG |
| | | GTGCTCCAACAAAAAAAATGCTGCTGTAATGGACCAAGAGCCTGCAGGGA |
| | | ACAGAACAGTGAACAGCGAGGATTCTGATGAACAAGACCATCAGGAGGT |
| | | GTCATACGCATAATGAGC |
| 733 | 18 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGGATCCCAGTCTGCGCTGACTCAAACG |
| | | GCCTCAGTGTCTGGGTCCCTGGGCCAGAAGGTCACCATCTCCTGCACTGG |
| | | AAGCAGGTCCAACATCGGTGCAAATTATGTGGGCTGGTATCAACAGGTCCC |
| | | AGGAATAGGCCCTAGAACCGTCATTTTTGGTTATAATTATCGACCTTCAGGG |
| | | GTCCCCGATCGATTCTCTAGCTCCAAGTCAGGCAATTCAGCCACCCTGACC |
| | | ATCTCTGGGCTCCAGGCTGAGGACGAGGCTGAGTATTATTGTTCAACATGG |
| | | GACAGCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCTCGGG |
| | | CGGTGGTGGGGAGGTGCCACTGGTGGAATCTGGGGGAGACCTGGTGAAG |
| | | CCTGGGGGGTCCTTGAGACTGTCCTGTGTGGCCTCTGGATTCACCCTCAGT |
| | | AACTATGGCATGAGCTGGGTCCGTCAGTCTCCAGGGAAGGGGCTGCAGTG |
| | | GGTCGCAGCTATTAGGAGTGGTGGAAACACATACTACGCAGACGCTGTGA |
| | | AGGGCCGATTCACCATCTCCAGAGACAACGCCAAGAACACGCTGTATCTG |
| | | CAGATGAACAGCCTGAGAGCCGAGGACACGGCCGTGTATTACTGTGCGTC |
| | | CCAATGGGGTGATTGGGGCCACTTTGAGTACTGGGGCCAGGGCACCCTGG |
| | | TCACCGTCTCCTCAGCTAGCGAGCAGAAATTAATCTCAGAGGAGGACCTG |
| | | CCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCT |
| | | CAGTGGTCAAAATCCCTTTCACCATCCTCCTCTTCTTTCTCCTTCATCGCTG |
| | | GTGCTCCAACAAAAAAAATGCTGCTGTAATGGACCAAGAGCCTGCAGGGA |
| | | ACAGAACAGTGAACAGCGAGGATTCTGATGAACAAGACCATCAGGAGGT |
| | | GTCATACGCAGTCGAGGGCGGCGGAGAGGGCAGAGGAAGTCTTCTAACAT |
| | | GCGGTGACGTGGAGGAGAATCCCGGCCCTAGGATGGCCTTACCAGTGACC |
| | | GCCTTGCTCCTGCCGCTGGCCTTGCTGCTCCACGCCGCCAGGCCGGAGGT |
| | | GCAGCTGCAGCAGTCTGGGGCTGAGCTGGTGAAGCCTGGGGCCTCAGTGA |
| | | AGATGTCCTGCAAGGCTTCTGGCTACACATTTACCAGTTACAATATGCACTG |
| | | GGTAAAGCAGACACCTGGACAGGGCCTGGAATGGATTGGAGCTATTTATCC |
| | | AGGAAATGGTGATACTTCCTACAATCAGAAGTTCAAAGGCAAGGCCACAT |
| | | TGACTGCAGACAAATCCTCCAGCACAGCCTACATGCAGCTCAGCAGCCTG |
| | | ACATCTGAGGACTCTGCGGACTATTACTGTGCAAGATCTAATTATTACGGTA |
| | | GTAGCTACTGGTTCTTCGATGTCTGGGGCGCAGGGACCACGGTCACCGTCT |
| | | CCTCAGGCAGTACTAGCGGTGGTGGCTCCGGGGGCGGTTCCGGTGGGGGC |
| | | GGCAGCAGCGACATTGTGCTGACCCAATCTCCAGCTATCCTGTCTGCATCT |
| | | CCAGGGGAGAAGGTCACAATGACTTGCAGGGCCAGCTCAAGTGTAAATTA |
| | | CATGGACTGGTACCAGAAGAAGCCAGGTTCCTCCCCCAAACCCTGGATTT |
| | | ATGCCACATCCAACCTGGCTTCTGGAGTCCCTGCTCGCTTCAGTGGCAGTG |
| | | GGTCTGGGACCTCTTACTCTCTCACAATCAGCAGAGTGGAGGCTGAAGAT |
| | | GCTGCCACTTATTACTGCCAGCAGTGGAGTTTTAATCCACCCACGTTCGGA |
| | | GGGGGGACCAAGCTGGAAATAAAAGGAGCTAGCCACCATCACCATCACCA |
| | | TCCTAGCGGTGGCGGAGGTTCTGGAGGTGGGGGTTCCTCACCCACTGAAC |
| | | CAAGCTCCAAAACCGGTAACCCCAGACACCTGCATGTTCTGATTGGGACC |
| | | TCAGTGGTCAAAATCCCTTTCACCATCCTCCTCTTCTTTCTCCTTCATCGCT |
| | | GGTGCTCCAACAAAAAAAATGCTGCTGTAATGGACCAAGAGCCTGCAGGG |
| | | AACAGAACAGTGAACAGCGAGGATTCTGATGAACAAGACCATCAGGAGG |
| | | TGTCATACGCATAATGAGC |
| 734 | 19 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGCCACGAACTTCTCTCTGTTAAAGCAAGCAGGAGACGTGGAAGAAA |
| | | ACCCCGGTCCTAGGATGGCCTTACCAGTGACCGCCTTGCTCCTGCCGCTGG |
| | | CCTTGCTGCTCCACGCCGCCAGGCCGGAGGTGCAGCTGCAGCAGTCTGGG |
| | | GCTGAGCTGGTGAAGCCTGGGGCCTCAGTGAAGATGTCCTGCAAGGCTTC |
| | | TGGCTACACATTTACCAGTTACAATATGCACTGGGTAAAGCAGACACCTGG |
| | | ACAGGGCCTGGAATGGATTGGAGCTATTTATCCAGGAAATGGTGATACTTC |
| | | CTACAATCAGAAGTTCAAAGGCAAGGCCACATTGACTGCAGACAAATCCT |
| | | CCAGCACAGCCTACATGCAGCTCAGCAGCCTGACATCTGAGGACTCTGCG |
| | | GACTATTACTGTGCAAGATCTAATTATTACGGTAGTAGCTACTGGTTCTTCG |
| | | ATGTCTGGGGCGCAGGGACCACGGTCACCGTCTCCTCAGGCAGTACTAGC |
| | | GGTGGTGGCTCCGGGGGCGGTTCCGGTGGGGGCGGCAGCAGCGACATTGT |
| | | GCTGACCCAATCTCCAGCTATCCTGTCTGCATCTCCAGGGGAGAAGGTCAC |
| | | AATGACTTGCAGGGCCAGCTCAAGTGTAAATTACATGGACTGGTACCAGA |
| | | AGAAGCCAGGTTCCTCCCCCAAACCCTGGATTTATGCCACATCCAACCTGG |
| | | CTTCTGGAGTCCCTGCTCGCTTCAGTGGCAGTGGGTCTGGGACCTCTTACT |
| | | CTCTCACAATCAGCAGAGTGGAGGCTGAAGATGCTGCCACTTATTACTGCC |
| | | AGCAGTGGAGTTTTAATCCACCCACGTTCGGAGGGGGGACCAAGCTGGAA |
| | | ATAAAAGGAGCTAGCCACCATCACCATCACCATCCTAGCGGTGGCGGAGGT |
| | | TCTGGAGGTGGGGGTTCCTCACCCACTGAACCAAGCTCCAAAACCGGTAA |
| | | CCCCAGACACCTGCATGTTCTGATTGGGACCTCAGTGGTCAAAATCCCTTT |
| | | CACCATCCTCCTCTTCTTTCTCCTTCATCGCTGGTGCTCCAACAAAAAAAAT |
| | | GCTGCTGTAATGGACCAAGAGCCTGCAGGGAACAGAACAGTGAACAGCG |
| | | AGGATTCTGATGAACAAGACCATCAGGAGGTGTCATACGCAGTCGAGGGC |
| | | GGCGGAGAGGGCAGAGGAAGTCTTCTAACATGCGGTGACGTGGAGGAGA |
| | | ATCCCGGCCCTAGGATGGCCTTACCAGTGACCGCCTTGCTCCTGCCGCTGG |
| | | CCTTGCTGCTCCACGCCGCCAGGCCGGGATCCCAGTCTGCGCTGACTCAA |
| | | ACGGCCTCAGTGTCTGGGTCCCTGGGCCAGAAGGTCACCATCTCCTGCAC |
| | | TGGAAGCAGGTCCAACATCGGTGCAAATTATGTGGGCTGGTATCAACAGGT |
| | | CCCAGGAATAGGCCCTAGAACCGTCATTTTTGGTTATAATTATCGACCTTCA |
| | | GGGGTCCCCGATCGATTCTCTAGCTCCAAGTCAGGCAATTCAGCCACCCTG |
| | | ACCATCTCTGGGCTCCAGGCTGAGGACGAGGCTGAGTATTATTGTTCAACA |
| | | TGGGACAGCAGTCTCAACGCTTACGTGTTCGGCTCAGGGACCCAGCTGAC |
| | | CGTCCTCGGCGGTGGTTCCTCTAGATCTTCCTCCTCTGGTGGCGGTGGCTC |
| | | GGGCGGTGGTGGGGAGGTGCCACTGGTGGAATCTGGGGGAGACCTGGTG |
| | | AAGCCTGGGGGGTCCTTGAGACTGTCCTGTGTGGCCTCTGGATTCACCCTC |
| | | AGTAACTATGGCATGAGCTGGGTCCGTCAGTCTCCAGGGAAGGGGCTGCA |
| | | GTGGGTCGCAGCTATTAGGAGTGGTGGAAACACATACTACGCAGACGCTG |
| | | TGAAGGGCCGATTCACCATCTCCAGAGACAACGCCAAGAACACGCTGTAT |
| | | CTGCAGATGAACAGCCTGAGAGCCGAGGACACGGCCGTGTATTACTGTGC |
| | | GTCCCAATGGGGTGATTGGGGCCACTTTGAGTACTGGGGCCAGGGCACCC |
| | | TGGTCACCGTCTCCTCAGCTAGCGAGCAGAAATTAATCTCAGAGGAGGAC |
| | | CTGCCTAGCGGTGGCGGAGGTTCTGGAGGTGGGGGTTCCTCACCCACTGA |
| | | ACCAAGCTCCAAAACCGGTAACCCCAGACACCTGCATGTTCTGATTGGGA |
| | | CCTCAGTGGTCAAAATCCCTTTCACCATCCTCCTCTTCTTTCTCCTTCATCG |
| | | CTGGTGCTCCAACAAAAAAAATGCTGCTGTAATGGACCAAGAGCCTGCAG |
| | | GGAACAGAACAGTGAACAGCGAGGATTCTGATGAACAAGACCATCAGGA |
| | | GGTGTCATACGCATAATGAGC |
| 735 | 20 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGCCACGAACTTCTCTCTGTTAAAGCAAGCAGGAGACGTGGAAGAAA |
| | | ACCCCGGTCCTAGGATGGCCTTACCAGTGACCGCCTTGCTCCTGCCGCTGG |
| | | CCTTGCTGCTCCACGCCGCCAGGCCGGGATCCCAGTCTGCGCTGACTCAA |
| | | ACGGCCTCAGTGTCTGGGTCCCTGGGCCAGAAGGTCACCATCTCCTGCAC |
| | | TGGAAGCAGGTCCAACATCGGTGCAAATTATGTGGGCTGGTATCAACAGGT |
| | | CCCAGGAATAGGCCCTAGAACCGTCATTTTTGGTTATAATTATCGACCTTCA |
| | | GGGGTCCCCGATCGATTCTCTAGCTCCAAGTCAGGCAATTCAGCCACCCTG |
| | | ACCATCTCTGGGCTCCAGGCTGAGGACGAGGCTGAGTATTATTGTTCAACA |
| | | TGGGACAGCAGTCTCAACGCTTACGTGTTCGGCTCAGGGACCCAGCTGAC |
| | | CGTCCTCGGCGGTGGTTCCTCTAGATCTTCCTCCTCTGGTGGCGGTGGCTC |
| | | GGGCGGTGGTGGGGAGGTGCCACTGGTGGAATCTGGGGGAGACCTGGTG |
| | | AAGCCTGGGGGGTCCTTGAGACTGTCCTGTGTGGCCTCTGGATTCACCCTC |
| | | AGTAACTATGGCATGAGCTGGGTCCGTCAGTCTCCAGGGAAGGGGCTGCA |
| | | GTGGGTCGCAGCTATTAGGAGTGGTGGAAACACATACTACGCAGACGCTG |
| | | TGAAGGGCCGATTCACCATCTCCAGAGACAACGCCAAGAACACGCTGTAT |
| | | CTGCAGATGAACAGCCTGAGAGCCGAGGACACGGCCGTGTATTACTGTGC |
| | | GTCCCAATGGGGTGATTGGGGCCACTTTGAGTACTGGGGCCAGGGCACCC |
| | | TGGTCACCGTCTCCTCAGCTAGCGAGCAGAAATTAATCTCAGAGGAGGAC |
| | | CTGCCTAGCGGTGGCGGAGGTTCTGGAGGTGGGGGTTCCTCACCCACTGA |
| | | ACCAAGCTCCAAAACCGGTAACCCCAGACACCTGCATGTTCTGATTGGGA |
| | | CCTCAGTGGTCAAAATCCCTTTCACCATCCTCCTCTTCTTTCTCCTTCATCG |
| | | CTGGTGCTCCAACAAAAAAAATGCTGCTGTAATGGACCAAGAGCCTGCAG |
| | | GGAACAGAACAGTGAACAGCGAGGATTCTGATGAACAAGACCATCAGGA |
| | | GGTGTCATACGCAGTCGAGGGCGGCGGAGAGGGCAGAGGAAGTCTTCTA |
| | | ACATGCGGTGACGTGGAGGAGAATCCCGGCCCTAGGATGGCCTTACCAGT |
| | | GACCGCCTTGCTCCTGCCGCTGGCCTTGCTGCTCCACGCCGCCAGGCCGG |
| | | AGGTGCAGCTGCAGCAGTCTGGGGCTGAGCTGGTGAAGCCTGGGGCCTCA |
| | | GTGAAGATGTCCTGCAAGGCTTCTGGCTACACATTTACCAGTTACAATATG |
| | | CACTGGGTAAAGCAGACACCTGGACAGGGCCTGGAATGGATTGGAGCTAT |
| | | TTATCCAGGAAATGGTGATACTTCCTACAATCAGAAGTTCAAAGGCAAGGC |
| | | CACATTGACTGCAGACAAATCCTCCAGCACAGCCTACATGCAGCTCAGCA |
| | | GCCTGACATCTGAGGACTCTGCGGACTATTACTGTGCAAGATCTAATTATTA |
| | | CGGTAGTAGCTACTGGTTCTTCGATGTCTGGGGCGCAGGGACCACGGTCAC |
| | | CGTCTCCTCAGGCAGTACTAGCGGTGGTGGCTCCGGGGGCGGTTCCGGTG |
| | | GGGGCGGCAGCAGCGACATTGTGCTGACCCAATCTCCAGCTATCCTGTCTG |
| | | CATCTCCAGGGGAGAAGGTCACAATGACTTGCAGGGCCAGCTCAAGTGTA |
| | | AATTACATGGACTGGTACCAGAAGAAGCCAGGTTCCTCCCCCAAACCCTG |
| | | GATTTATGCCACATCCAACCTGGCTTCTGGAGTCCCTGCTCGCTTCAGTGG |
| | | CAGTGGGTCTGGGACCTCTTACTCTCTCACAATCAGCAGAGTGGAGGCTG |
| | | AAGATGCTGCCACTTATTACTGCCAGCAGTGGAGTTTTAATCCACCCACGT |
| | | TCGGAGGGGGGACCAAGCTGGAAATAAAAGGAGCTAGCCACCATCACCAT |
| | | CACCATCCTAGCGGTGGCGGAGGTTCTGGAGGTGGGGGTTCCTCACCCAC |
| | | TGAACCAAGCTCCAAAACCGGTAACCCCAGACACCTGCATGTTCTGATTG |
| | | GGACCTCAGTGGTCAAAATCCCTTTCACCATCCTCCTCTTCTTTCTCCTTCA |
| | | TCGCTGGTGCTCCAACAAAAAAAATGCTGCTGTAATGGACCAAGAGCCTG |
| | | CAGGGAACAGAACAGTGAACAGCGAGGATTCTGATGAACAAGACCATCA |
| | | GGAGGTGTCATACGCATAATGAGC |
| 736 | 21 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCCTTACCAGTGACCGCCTTGCTCCTGCCGCTGGCCT |
| | | TGCTGCTCCACGCCGCCAGGCCGGGATCCCAGTCTGCGCTGACTCAAACG |
| | | GCCTCAGTGTCTGGGTCCCTGGGCCAGAAGGTCACCATCTCCTGCACTGG |
| | | AAGCAGGTCCAACATCGGTGCAAATTATGTGGGCTGGTATCAACAGGTCCC |
| | | AGGAATAGGCCCTAGAACCGTCATTTTTGGTTATAATTATCGACCTTCAGGG |
| | | GTCCCCGATCGATTCTCTAGCTCCAAGTCAGGCAATTCAGCCACCCTGACC |
| | | ATCTCTGGGCTCCAGGCTGAGGACGAGGCTGAGTATTATTGTTCAACATGG |
| | | GACAGCAGTCTCAACGCTTACGTGTTCGGCTCAGGGACCCAGCTGACCGT |
| | | CCTCGGCGGTGGTTCCTCTAGATCTTCCTCCTCTGGTGGCGGTGGCTCGGG |
| | | CGGTGGTGGGGAGGTGCCACTGGTGGAATCTGGGGGAGACCTGGTGAAG |
| | | CCTGGGGGGTCCTTGAGACTGTCCTGTGTGGCCTCTGGATTCACCCTCAGT |
| | | AACTATGGCATGAGCTGGGTCCGTCAGTCTCCAGGGAAGGGGCTGCAGTG |
| | | GGTCGCAGCTATTAGGAGTGGTGGAAACACATACTACGCAGACGCTGTGA |
| | | AGGGCCGATTCACCATCTCCAGAGACAACGCCAAGAACACGCTGTATCTG |
| | | CAGATGAACAGCCTGAGAGCCGAGGACACGGCCGTGTATTACTGTGCGTC |
| | | CCAATGGGGTGATTGGGGCCACTTTGAGTACTGGGGCCAGGGCACCCTGG |
| | | TCACCGTCTCCTCAGCTAGCGAGCAGAAATTAATCTCAGAGGAGGACCTG |
| | | CCTAGCGGTGGCGGAGGTTCTGGAGGTGGGGGTTCCTCACCCACTGAACC |
| | | AAGCTCCAAAACCGGTAACCCCAGACACCTGCATGTTCTGATTGGGACCT |
| | | CAGTGGTCAAAATCCCTTTCACCATCCTCCTCTTCTTTCTCCTTCATCGCTG |
| | | GTGCTCCAACAAAAAAAATGCTGCTGTAATGGACCAAGAGCCTGCAGGGA |
| | | ACAGAACAGTGAACAGCGAGGATTCTGATGAACAAGACCATCAGGAGGT |
| | | GTCATACGCAGTCGAGGGCGGCGGAGCCACGAACTTCTCTCTGTTAAAGC |
| | | AAGCAGGAGACGTGGAAGAAAACCCCGGTCCTAGGATGGCACTCCCCGTC |
| | | ACCGCGCTCCTCTTGCCCCTCGCGCTGCTCCTTCATGCTGCGAGGCCGGAG |
| | | GTGCAGCTGCAGCAGTCTGGGGCTGAGCTGGTGAAGCCTGGGGCCTCAGT |
| | | GAAGATGTCCTGCAAGGCTTCTGGCTACACATTTACCAGTTACAATATGCA |
| | | CTGGGTAAAGCAGACACCTGGACAGGGCCTGGAATGGATTGGAGCTATTT |
| | | ATCCAGGAAATGGTGATACTTCCTACAATCAGAAGTTCAAAGGCAAGGCC |
| | | ACATTGACTGCAGACAAATCCTCCAGCACAGCCTACATGCAGCTCAGCAG |
| | | CCTGACATCTGAGGACTCTGCGGACTATTACTGTGCAAGATCTAATTATTAC |
| | | GGTAGTAGCTACTGGTTCTTCGATGTCTGGGGCGCAGGGACCACGGTCACC |
| | | GTCTCCTCAGGCAGTACTAGCGGTGGTGGCTCCGGGGGCGGTTCCGGTGG |
| | | GGGCGGCAGCAGCGACATTGTGCTGACCCAATCTCCAGCTATCCTGTCTGC |
| | | ATCTCCAGGGGAGAAGGTCACAATGACTTGCAGGGCCAGCTCAAGTGTAA |
| | | ATTACATGGACTGGTACCAGAAGAAGCCAGGTTCCTCCCCCAAACCCTGG |
| | | ATTTATGCCACATCCAACCTGGCTTCTGGAGTCCCTGCTCGCTTCAGTGGC |
| | | AGTGGGTCTGGGACCTCTTACTCTCTCACAATCAGCAGAGTGGAGGCTGA |
| | | AGATGCTGCCACTTATTACTGCCAGCAGTGGAGTTTTAATCCACCCACGTT |
| | | CGGAGGGGGGACCAAGCTGGAAATAAAACCTAGCGGAGGCGGCGGCAGC |
| | | GGCGGGGGAGGGTCCGGAGGGGGTGGTTCTGGTGGAGGAGGATCGGGAG |
| | | GCGGTGGCAGCCCCTATGAGTGGTCAAACTCGAGTGACCCACTGCTTGTTT |
| | | CTGTCACAGGAAACCCTTCAAATAGTTGGCCTTCACCAACAGAGCCGTCA |
| | | AGTAAAACCGGGAACCCGAGACATCTCCACGTTCTGATCGGAACATCCGT |
| | | CGTAAAAATCCCTTTCACCATATTGTTGTTTTTCTTGTTGCACCGATGGTGC |
| | | TCTAACAAGAAGAACGCAGCCGTAATGGACCAAGAGCCAGCCGGAAACA |
| | | GGACTGTCAATTCCGAAGATTCCGATGAGCAGGATCACCAAGAAGTTAGC |
| | | TACGCATAA |
| 737 | 22 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGCCACGAACTTCTCTCTGTTAAAGCAAGCAGGAGACGTGGAAGAAA |
| | | ACCCCGGTCCTAGGATGGCCTTACCAGTGACCGCCTTGCTCCTGCCGCTGG |
| | | CCTTGCTGCTCCACGCCGCCAGGCCGGGATCCCAGTCTGCGCTGACTCAA |
| | | ACGGCCTCAGTGTCTGGGTCCCTGGGCCAGAAGGTCACCATCTCCTGCAC |
| | | TGGAAGCAGGTCCAACATCGGTGCAAATTATGTGGGCTGGTATCAACAGGT |
| | | CCCAGGAATAGGCCCTAGAACCGTCATTTTTGGTTATAATTATCGACCTTCA |
| | | GGGGTCCCCGATCGATTCTCTAGCTCCAAGTCAGGCAATTCAGCCACCCTG |
| | | ACCATCTCTGGGCTCCAGGCTGAGGACGAGGCTGAGTATTATTGTTCAACA |
| | | TGGGACAGCAGTCTCAACGCTTACGTGTTCGGCTCAGGGACCCAGCTGAC |
| | | CGTCCTCGGCGGTGGTTCCTCTAGATCTTCCTCCTCTGGTGGCGGTGGCTC |
| | | GGGCGGTGGTGGGGAGGTGCCACTGGTGGAATCTGGGGGAGACCTGGTG |
| | | AAGCCTGGGGGGTCCTTGAGACTGTCCTGTGTGGCCTCTGGATTCACCCTC |
| | | AGTAACTATGGCATGAGCTGGGTCCGTCAGTCTCCAGGGAAGGGGCTGCA |
| | | GTGGGTCGCAGCTATTAGGAGTGGTGGAAACACATACTACGCAGACGCTG |
| | | TGAAGGGCCGATTCACCATCTCCAGAGACAACGCCAAGAACACGCTGTAT |
| | | CTGCAGATGAACAGCCTGAGAGCCGAGGACACGGCCGTGTATTACTGTGC |
| | | GTCCCAATGGGGTGATTGGGGCCACTTTGAGTACTGGGGCCAGGGCACCC |
| | | TGGTCACCGTCTCCTCAGCTAGCGAGCAGAAATTAATCTCAGAGGAGGAC |
| | | CTGCCTAGCGGTGGCGGAGGTTCTGGAGGTGGGGGTTCCTCACCCACTGA |
| | | ACCAAGCTCCAAAACCGGTAACCCCAGACACCTGCATGTTCTGATTGGGA |
| | | CCTCAGTGGTCAAAATCCCTTTCACCATCCTCCTCTTCTTTCTCCTTCATCG |
| | | CTGGTGCTCCAACAAAAAAAATGCTGCTGTAATGGACCAAGAGCCTGCAG |
| | | GGAACAGAACAGTGAACAGCGAGGATTCTGATGAACAAGACCATCAGGA |
| | | GGTGTCATACGCAGTCGAGGGCGGCGGAGAGGGCAGAGGAAGTCTTCTA |
| | | ACATGCGGTGACGTGGAGGAGAATCCCGGCCCTAGGATGGCACTCCCCGT |
| | | CACCGCGCTCCTCTTGCCCCTCGCGCTGCTCCTTCATGCTGCGAGGCCGGA |
| | | GGTGCAGCTGCAGCAGTCTGGGGCTGAGCTGGTGAAGCCTGGGGCCTCAG |
| | | TGAAGATGTCCTGCAAGGCTTCTGGCTACACATTTACCAGTTACAATATGC |
| | | ACTGGGTAAAGCAGACACCTGGACAGGGCCTGGAATGGATTGGAGCTATT |
| | | TATCCAGGAAATGGTGATACTTCCTACAATCAGAAGTTCAAAGGCAAGGCC |
| | | ACATTGACTGCAGACAAATCCTCCAGCACAGCCTACATGCAGCTCAGCAG |
| | | CCTGACATCTGAGGACTCTGCGGACTATTACTGTGCAAGATCTAATTATTAC |
| | | GGTAGTAGCTACTGGTTCTTCGATGTCTGGGGCGCAGGGACCACGGTCACC |
| | | GTCTCCTCAGGCAGTACTAGCGGTGGTGGCTCCGGGGGCGGTTCCGGTGG |
| | | GGGCGGCAGCAGCGACATTGTGCTGACCCAATCTCCAGCTATCCTGTCTGC |
| | | ATCTCCAGGGGAGAAGGTCACAATGACTTGCAGGGCCAGCTCAAGTGTAA |
| | | ATTACATGGACTGGTACCAGAAGAAGCCAGGTTCCTCCCCCAAACCCTGG |
| | | ATTTATGCCACATCCAACCTGGCTTCTGGAGTCCCTGCTCGCTTCAGTGGC |
| | | AGTGGGTCTGGGACCTCTTACTCTCTCACAATCAGCAGAGTGGAGGCTGA |
| | | AGATGCTGCCACTTATTACTGCCAGCAGTGGAGTTTTAATCCACCCACGTT |
| | | CGGAGGGGGGACCAAGCTGGAAATAAAACCTAGCGGAGGCGGCGGCAGC |
| | | GGCGGGGGAGGGTCCGGAGGGGGTGGTTCTGGTGGAGGAGGATCGGGAG |
| | | GCGGTGGCAGCCCCTATGAGTGGTCAAACTCGAGTGACCCACTGCTTGTTT |
| | | CTGTCACAGGAAACCCTTCAAATAGTTGGCCTTCACCAACAGAGCCGTCA |
| | | AGTAAAACCGGGAACCCGAGACATCTCCACGTTCTGATCGGAACATCCGT |
| | | CGTAAAAATCCCTTTCACCATATTGTTGTTTTTCTTGTTGCACCGATGGTGC |
| | | TCTAACAAGAAGAACGCAGCCGTAATGGACCAAGAGCCAGCCGGAAACA |
| | | GGACTGTCAATTCCGAAGATTCCGATGAGCAGGATCACCAAGAAGTTAGC |
| | | TACGCATAA |
| 738 | 23 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGAGGGCAGAGGAAGTCTTCTAACATGCGGTGACGTGGAGGAGAATC |
| | | CCGGCCCTAGGATGGCACTCCCCGTCACCGCGCTCCTCTTGCCCCTCGCGC |
| | | TGCTCCTTCATGCTGCGAGGCCGGAGGTGCAGCTGCAGCAGTCTGGGGCT |
| | | GAGCTGGTGAAGCCTGGGGCCTCAGTGAAGATGTCCTGCAAGGCTTCTGG |
| | | CTACACATTTACCAGTTACAATATGCACTGGGTAAAGCAGACACCTGGACA |
| | | GGGCCTGGAATGGATTGGAGCTATTTATCCAGGAAATGGTGATACTTCCTAC |
| | | AATCAGAAGTTCAAAGGCAAGGCCACATTGACTGCAGACAAATCCTCCAG |
| | | CACAGCCTACATGCAGCTCAGCAGCCTGACATCTGAGGACTCTGCGGACT |
| | | ATTACTGTGCAAGATCTAATTATTACGGTAGTAGCTACTGGTTCTTCGATGTC |
| | | TGGGGCGCAGGGACCACGGTCACCGTCTCCTCAGGCAGTACTAGCGGTGG |
| | | TGGCTCCGGGGGCGGTTCCGGTGGGGGCGGCAGCAGCGACATTGTGCTGA |
| | | CCCAATCTCCAGCTATCCTGTCTGCATCTCCAGGGGAGAAGGTCACAATGA |
| | | CTTGCAGGGCCAGCTCAAGTGTAAATTACATGGACTGGTACCAGAAGAAG |
| | | CCAGGTTCCTCCCCCAAACCCTGGATTTATGCCACATCCAACCTGGCTTCT |
| | | GGAGTCCCTGCTCGCTTCAGTGGCAGTGGGTCTGGGACCTCTTACTCTCTC |
| | | ACAATCAGCAGAGTGGAGGCTGAAGATGCTGCCACTTATTACTGCCAGCA |
| | | GTGGAGTTTTAATCCACCCACGTTCGGAGGGGGGACCAAGCTGGAAATAA |
| | | AACCTAGCGGAGGCGGCGGCAGCGGCGGGGGAGGGTCCGGAGGGGGTGG |
| | | TTCTGGTGGAGGAGGATCGGGAGGCGGTGGCAGCCCCTATGAGTGGTCAA |
| | | ACTCGAGTGACCCACTGCTTGTTTCTGTCACAGGAAACCCTTCAAATAGTT |
| | | GGCCTTCACCAACAGAGCCGTCAAGTAAAACCGGGAACCCGAGACATCTC |
| | | CACGTTCTGATCGGAACATCCGTCGTAAAAATCCCTTTCACCATATTGTTGT |
| | | TTTTCTTGTTGCACCGATGGTGCTCTAACAAGAAGAACGCAGCCGTAATGG |
| | | ACCAAGAGCCAGCCGGAAACAGGACTGTCAATTCCGAAGATTCCGATGAG |
| | | CAGGATCACCAAGAAGTTAGCTACGCAGTCGAGGGCGGCGGAGCCACGA |
| | | ACTTCTCTCTGTTAAAGCAAGCAGGAGACGTGGAAGAAAACCCCGGTCCT |
| | | AGGATGGCCTTACCAGTGACCGCCTTGCTCCTGCCGCTGGCCTTGCTGCTC |
| | | CACGCCGCCAGGCCGGGATCCCAGTCTGCGCTGACTCAAACGGCCTCAGT |
| | | GTCTGGGTCCCTGGGCCAGAAGGTCACCATCTCCTGCACTGGAAGCAGGT |
| | | CCAACATCGGTGCAAATTATGTGGGCTGGTATCAACAGGTCCCAGGAATAG |
| | | GCCCTAGAACCGTCATTTTTGGTTATAATTATCGACCTTCAGGGGTCCCCGA |
| | | TCGATTCTCTAGCTCCAAGTCAGGCAATTCAGCCACCCTGACCATCTCTGG |
| | | GCTCCAGGCTGAGGACGAGGCTGAGTATTATTGTTCAACATGGGACAGCA |
| | | GTCTCAACGCTTACGTGTTCGGCTCAGGGACCCAGCTGACCGTCCTCGGC |
| | | GGTGGTTCCTCTAGATCTTCCTCCTCTGGTGGCGGTGGCTCGGGCGGTGGT |
| | | GGGGAGGTGCCACTGGTGGAATCTGGGGGAGACCTGGTGAAGCCTGGGG |
| | | GGTCCTTGAGACTGTCCTGTGTGGCCTCTGGATTCACCCTCAGTAACTATG |
| | | GCATGAGCTGGGTCCGTCAGTCTCCAGGGAAGGGGCTGCAGTGGGTCGCA |
| | | GCTATTAGGAGTGGTGGAAACACATACTACGCAGACGCTGTGAAGGGCCG |
| | | ATTCACCATCTCCAGAGACAACGCCAAGAACACGCTGTATCTGCAGATGA |
| | | ACAGCCTGAGAGCCGAGGACACGGCCGTGTATTACTGTGCGTCCCAATGG |
| | | GGTGATTGGGGCCACTTTGAGTACTGGGGCCAGGGCACCCTGGTCACCGT |
| | | CTCCTCAGCTAGCGAGCAGAAATTAATCTCAGAGGAGGACCTGCCTAGCG |
| | | GTGGCGGAGGTTCTGGAGGTGGGGGTTCCTCACCCACTGAACCAAGCTCC |
| | | AAAACCGGTAACCCCAGACACCTGCATGTTCTGATTGGGACCTCAGTGGT |
| | | CAAAATCCCTTTCACCATCCTCCTCTTCTTTCTCCTTCATCGCTGGTGCTCC |
| | | AACAAAAAAAATGCTGCTGTAATGGACCAAGAGCCTGCAGGGAACAGAA |
| | | CAGTGAACAGCGAGGATTCTGATGAACAAGACCATCAGGAGGTGTCATAC |
| | | GCATAA |
| 739 | 24 | ATGGGGGGACTTGAACCCTGCAGCAGGTTCCTGCTCCTGCCTCTCCTGCTG |
| | DNA | GCTGTAAGTGGTCTCCGTCCTGTCCAGGTCCAGGCCCAGAGCGATTGCAG |
| | | TTGCTCTACGGTGAGCCCGGGCGTGCTGGCAGGGATCGTGATGGGAGACC |
| | | TGGTGCTGACAGTGCTCATTGCCCTGGCCGTGTACTTCCTGGGCCGGCTGG |
| | | TCCCTCGGGGGCGAGGGGCTGCGGAGGCAGCGACCCGGAAACAGCGTAT |
| | | CACTGAGACCGAGTCGCCTTATCAGGAGCTCCAGGGTCAGAGGTCGGATG |
| | | TCTACAGCGACCTCAACACACAGAGGCCGTATTACAAAGTCGAGGGCGGC |
| | | GGAGCCACGAACTTCTCTCTGTTAAAGCAAGCAGGAGACGTGGAAGAAA |
| | | ACCCCGGTCCTAGGATGGCACTCCCCGTCACCGCGCTCCTCTTGCCCCTCG |
| | | CGCTGCTCCTTCATGCTGCGAGGCCGGAGGTGCAGCTGCAGCAGTCTGGG |
| | | GCTGAGCTGGTGAAGCCTGGGGCCTCAGTGAAGATGTCCTGCAAGGCTTC |
| | | TGGCTACACATTTACCAGTTACAATATGCACTGGGTAAAGCAGACACCTGG |
| | | ACAGGGCCTGGAATGGATTGGAGCTATTTATCCAGGAAATGGTGATACTTC |
| | | CTACAATCAGAAGTTCAAAGGCAAGGCCACATTGACTGCAGACAAATCCT |
| | | CCAGCACAGCCTACATGCAGCTCAGCAGCCTGACATCTGAGGACTCTGCG |
| | | GACTATTACTGTGCAAGATCTAATTATTACGGTAGTAGCTACTGGTTCTTCG |
| | | ATGTCTGGGGCGCAGGGACCACGGTCACCGTCTCCTCAGGCAGTACTAGC |
| | | GGTGGTGGCTCCGGGGGCGGTTCCGGTGGGGGCGGCAGCAGCGACATTGT |
| | | GCTGACCCAATCTCCAGCTATCCTGTCTGCATCTCCAGGGGAGAAGGTCAC |
| | | AATGACTTGCAGGGCCAGCTCAAGTGTAAATTACATGGACTGGTACCAGA |
| | | AGAAGCCAGGTTCCTCCCCCAAACCCTGGATTTATGCCACATCCAACCTGG |
| | | CTTCTGGAGTCCCTGCTCGCTTCAGTGGCAGTGGGTCTGGGACCTCTTACT |
| | | CTCTCACAATCAGCAGAGTGGAGGCTGAAGATGCTGCCACTTATTACTGCC |
| | | AGCAGTGGAGTTTTAATCCACCCACGTTCGGAGGGGGGACCAAGCTGGAA |
| | | ATAAAACCTAGCGGAGGCGGCGGCAGCGGCGGGGGAGGGTCCGGAGGGG |
| | | GTGGTTCTGGTGGAGGAGGATCGGGAGGCGGTGGCAGCCCCTATGAGTGG |
| | | TCAAACTCGAGTGACCCACTGCTTGTTTCTGTCACAGGAAACCCTTCAAAT |
| | | AGTTGGCCTTCACCAACAGAGCCGTCAAGTAAAACCGGGAACCCGAGAC |
| | | ATCTCCACGTTCTGATCGGAACATCCGTCGTAAAAATCCCTTTCACCATATT |
| | | GTTGTTTTTCTTGTTGCACCGATGGTGCTCTAACAAGAAGAACGCAGCCGT |
| | | AATGGACCAAGAGCCAGCCGGAAACAGGACTGTCAATTCCGAAGATTCCG |
| | | ATGAGCAGGATCACCAAGAAGTTAGCTACGCAGTCGAGGGCGGCGGAGA |
| | | GGGCAGAGGAAGTCTTCTAACATGCGGTGACGTGGAGGAGAATCCCGGCC |
| | | CTAGGATGGCCTTACCAGTGACCGCCTTGCTCCTGCCGCTGGCCTTGCTGC |
| | | TCCACGCCGCCAGGCCGGGATCCCAGTCTGCGCTGACTCAAACGGCCTCA |
| | | GTGTCTGGGTCCCTGGGCCAGAAGGTCACCATCTCCTGCACTGGAAGCAG |
| | | GTCCAACATCGGTGCAAATTATGTGGGCTGGTATCAACAGGTCCCAGGAAT |
| | | AGGCCCTAGAACCGTCATTTTTGGTTATAATTATCGACCTTCAGGGGTCCCC |
| | | GATCGATTCTCTAGCTCCAAGTCAGGCAATTCAGCCACCCTGACCATCTCT |
| | | GGGCTCCAGGCTGAGGACGAGGCTGAGTATTATTGTTCAACATGGGACAG |
| | | CAGTCTCAACGCTTACGTGTTCGGCTCAGGGACCCAGCTGACCGTCCTCG |
| | | GCGGTGGTTCCTCTAGATCTTCCTCCTCTGGTGGCGGTGGCTCGGGCGGTG |
| | | GTGGGGAGGTGCCACTGGTGGAATCTGGGGGAGACCTGGTGAAGCCTGG |
| | | GGGGTCCTTGAGACTGTCCTGTGTGGCCTCTGGATTCACCCTCAGTAACTA |
| | | TGGCATGAGCTGGGTCCGTCAGTCTCCAGGGAAGGGGCTGCAGTGGGTCG |
| | | CAGCTATTAGGAGTGGTGGAAACACATACTACGCAGACGCTGTGAAGGGC |
| | | CGATTCACCATCTCCAGAGACAACGCCAAGAACACGCTGTATCTGCAGATG |
| | | AACAGCCTGAGAGCCGAGGACACGGCCGTGTATTACTGTGCGTCCCAATG |
| | | GGGTGATTGGGGCCACTTTGAGTACTGGGGCCAGGGCACCCTGGTCACCG |
| | | TCTCCTCAGCTAGCGAGCAGAAATTAATCTCAGAGGAGGACCTGCCTAGC |
| | | GGTGGCGGAGGTTCTGGAGGTGGGGGTTCCTCACCCACTGAACCAAGCTC |
| | | CAAAACCGGTAACCCCAGACACCTGCATGTTCTGATTGGGACCTCAGTGG |
| | | TCAAAATCCCTTTCACCATCCTCCTCTTCTTTCTCCTTCATCGCTGGTGCTC |
| | | CAACAAAAAAAATGCTGCTGTAATGGACCAAGAGCCTGCAGGGAACAGA |
| | | ACAGTGAACAGCGAGGATTCTGATGAACAAGACCATCAGGAGGTGTCATA |
| | | CGCATAA |
| 810 | DAP12- | ATGGGGGGACTTGAACCCTGCAGCAGGTTCCTGCTCCTGCCTCTCCTGCT |
| | T2A- | GGCTGTAAGTGGTCTCCGTCCTGTCCAGGTCCAGGCCCAGAGCGATTGCA |
| | DS191- | GTTGCTCTACGGTGAGCCCGGGCGTGCTGGCAGGGATCGTGATGGGAGAC |
| | KIRShort- | CTGGTGCTGACAGTGCTCATTGCCCTGGCCGTGTACTTCCTGGGCCGGCT |
| | P2A-Leu16- | GGTCCCTCGGGGGCGAGGGGCTGCGGAGGCAGCGACCCGGAAACAGCGT |
| | KIRLong | ATCACTGAGACCGAGTCGCCTTATCAGGAGCTCCAGGGTCAGAGGTCGGA |
| | Codon opt | TGTCTACAGCGACCTCAACACACAGAGGCCGTATTACAAAGTCGAGGGCG |
| | | GCGGAGAGGGCAGAGGAAGTCTTCTAACATGCGGTGACGTGGAGGAGAA |
| | | TCCCGGCCCTAGGATGGCCTTACCAGTGACCGCCTTGCTCCTGCCGCTGGC |
| | | CTTGCTGCTCCACGCCGCCAGGCCGGGATCCCAGTCTGCGCTGACTCAAA |
| | | CGGCCTCAGTGTCTGGGTCCCTGGGCCAGAAGGTCACCATCTCCTGCACT |
| | | GGAAGCAGGTCCAACATCGGTGCAAATTATGTGGGCTGGTATCAACAGGT |
| | | CCCAGGAATAGGCCCTAGAACCGTCATTTTTGGTTATAATTATCGACCTTC |
| | | AGGGGTCCCCGATCGATTCTCTAGCTCCAAGTCAGGCAATTCAGCCACCC |
| | | TGACCATCTCTGGGCTCCAGGCTGAGGACGAGGCTGAGTATTATTGTTCA |
| | | ACATGGGACAGCAGTCTCAACGCTTACGTGTTCGGCTCAGGGACCCAGCT |
| | | GACCGTCCTCGGCGGTGGTTCCTCTAGATCTTCCTCCTCTGGTGGCGGTG |
| | | GCTCGGGCGGTGGTGGGGAGGTGCCACTGGTGGAATCTGGGGGAGACCT |
| | | GGTGAAGCCTGGGGGGTCCTTGAGACTGTCCTGTGTGGCCTCTGGATTCA |
| | | CCCTCAGTAACTATGGCATGAGCTGGGTCCGTCAGTCTCCAGGGAAGGGG |
| | | CTGCAGTGGGTCGCAGCTATTAGGAGTGGTGGAAACACATACTACGCAGA |
| | | CGCTGTGAAGGGCCGATTCACCATCTCCAGAGACAACGCCAAGAACACGC |
| | | TGTATCTGCAGATGAACAGCCTGAGAGCCGAGGACACGGCCGTGTATTACT |
| | | GTGCGTCCCAATGGGGTGATTGGGGCCACTTTGAGTACTGGGGCCAGGGC |
| | | ACCCTGGTCACCGTCTCCTCAGCTAGCGAGCAGAAATTAATCTCAGAGGA |
| | | GGACCTGCCTAGCGGTGGCGGAGGTTCTGGAGGTGGGGGTTCCTCACCCA |
| | | CTGAACCAAGCTCCAAAACCGGTAACCCCAGACACCTGCATGTTCTGATT |
| | | GGGACCTCAGTGGTCAAAATCCCTTTCACCATCCTCCTCTTCTTTCTCCT |
| | | TCATCGCTGGTGCTCCAACAAAAAAAATGCTGCTGTAATGGACCAAGAGC |
| | | CTGCAGGGAACAGAACAGTGAACAGCGAGGATTCTGATGAACAAGACCAT |
| | | CAGGAGGTGTCATACGCAGTCGAGGGCGGCGGAGCCACGAACTTCTCTCT |
| | | GTTAAAGCAAGCAGGAGACGTGGAAGAAAACCCCGGTCCTAGGATGGCA |
| | | CTCCCCGTCACCGCGCTCCTCTTGCCCCTCGCGCTGCTCCTTCATGCTGCG |
| | | AGGCCGGAGGTGCAGCTGCAGCAGTCTGGGGCTGAGCTGGTGAAGCCTG |
| | | GGGCCTCAGTGAAGATGTCCTGCAAGGCTTCTGGCTACACATTTACCAGTT |
| | | ACAATATGCACTGGGTAAAGCAGACACCTGGACAGGGCCTGGAATGGATT |
| | | GGAGCTATTTATCCAGGAAATGGTGATACTTCCTACAATCAGAAGTTCAA |
| | | AGGCAAGGCCACATTGACTGCAGACAAATCCTCCAGCACAGCCTACATGC |
| | | AGCTCAGCAGCCTGACATCTGAGGACAGCGCGGATTACTACTGCGCTAGG |
| | | TCCAATTATTATGGCTCCTCATACTGGTTCTTCGATGTCTGGGGCGCA |
| | | GGGACCACGGTCACCGTCTCCTCAGGCAGTACTAGCGGTGGTGGCTCCGG |
| | | GGGCGGTTCCGGTGGGGGCGGCAGCAGCGACATTGTGCTGACCCAATCTC |
| | | CAGCTATCCTGTCTGCATCTCCAGGGGAGAAGGTCACAATGACTTGCAGG |
| | | GCCAGCTCAAGTGTAAATTACATGGACTGGTACCAGAAGAAGCCAGGATC |
| | | CTCCCCCAAACCCTGGATTTATGCCACATCCAACCTGGCATCTGGAGTCC |
| | | CTGCTCGCTTCAGTGGCAGTGGGTCTGGGACCTCTTACTCTCTCACAATC |
| | | AGCAGAGTGGAGGCTGAAGATGCTGCCACTTATTACTGCCAGCAGTGGAG |
| | | TTTTAATCCACCCACGTTCGGAGGGGGGACCAAGCTGGAAATAAAACCTA |
| | | GCGGAGGCGGTGGCAGCGGCGGTGGAGGGTCAGGAGGGGGTGGTTCTGG |
| | | TGGAGGAGGATCGGGAGGCGGTGGAAGCCCATACGAATGGTCTAATTCCT |
| | | CTGACCCTCTCCTTGTGTCCGTGACCGGGAATCCTTCCAACTCCTGGCCTT |
| | | CACCAACAGAGCCGTCAAGTAAAACCGGGAACCCGAGACATCTCCACGTT |
| | | CTGATCGGAACATCCGTCGTAAAAATCCCTTTCACCATATTGTTGTTTTT |
| | | CTTGTTGCACCGATGGTGCTCTAACAAGAAGAACGCAGCCGTAATGGACC |
| | | AAGAGCCAGCCGGAAACAGGACTGTCAATTCCGAAGATTCCGATGAGCAG |
| | | GATCACCAAGAAGTTAGCTACGCA |

In Table 12, elements of each fusion protein include the following: 9—DS191-KIRshort; 10—Leu16-(G4S)2-KIRshort; 11—Leu16-(G4S)5-KIRshort; 12—Leu16-KIRlong; 13—DAP12-T2A-DS191-KIRshort-P2A-Leu16-KIRshort; 14—DAP12-T2A-Leu16-KIRshort-P2A-DS191-KIRshort; 15—DAP12-P2A-Leu16-KIRshort-P2A-DS191-KIRshort; 16—DAP12-P2A-DS191-KIRshort-P2A-Leu16-KIRshort; 17—DAP12-T2A-Leu16-KIRshort-T2A-DS191-KIRshort; 18—DAP12-T2A-DS191-KIRshort-T2A-Leu16-KIRshort; 19—DAP12-P2A-Leu16-KIRshort-T2A-DS191-KIRshort; 20—DAP12-P2A-DS191-KIRshort-T2A-Leu16-KIRshort; 21—DAP12-T2A-DS191-KIRshort-P2A-Leu16-KIRlong; 22—DAP12-P2A-DS191-KIRshort-T2A-Leu16-KIRlong; 23—DAP12-T2A-Leu16-KIRlong-P2A-DS191-KIRshort; and 24—DAP12-P2A-Leu16-KIRlong-T2A-DS191-KIRshort.

In some embodiments, the disclosure relates to a nucleic acid sequence encoding any amino acid sequence herein. In some embodiments, the disclosure relates to a nucleic acid sequence encoding any polypeptide herein. In some embodiments, the disclosure relates to a nucleic acid sequence encoding any fusion protein herein. In some embodiments, the disclosure relates to a composition comprising any nucleic acid sequence, nucleic acid molecule, vector, DNA, or RNA herein.

In some embodiments, a nucleic acid molecule comprises one or more nucleic acid sequence herein. In some embodiments, the nucleic acid molecule comprises a vector. In some embodiments, the vector comprises the backbone sequence of SEQ ID NO: 796, 797, or 798, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 796, 797, or 798. See Table 9. In some embodiments a nucleic acid molecule comprises any one or more nucleic acid sequence herein and a vector backbone sequence of one of SEQ ID NO: 796, 797, or 798, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 796, 797, or 798. In some embodiments, the backbone sequence comprises a vector from WO 2019/028051, WO 2016/100232, or WO 2018/067992, all of which are incorporated herein by reference in their entireties. In some embodiments, the backbone comprises a vector derived from at least a portion of a lentivirus genome, including especially a self-inactivating lentiviral vector as provided in Milone et al., Mol. Ther. 17(8): 1453-1464 (2009), which is incorporated herein by reference in its entirety. Other examples of lentivirus vectors that may be used in the clinic in some embodiments herein, include but are not limited to, e.g., the LENTIVECTOR® gene delivery technology from Oxford BioMedica, the LENTIMAX™ vector system from Lentigen and the like.

The nucleic acid molecule may be in an expression cassette or expression vector (e.g. a plasmid for introduction into a bacterial host cell, or a viral vector, or a plasmid or viral vector such as a lentivirus for transfection of a mammalian host cell). Suitable lentiviral vectors may be derived from HIV-1, HIV-2, SIV, FIV, BIV, EIAV, CAEV or visna lentivirus and are described in U.S. Pat. Nos. 6,924,123, 7,056,699, WO1998/055607, WO 1998/17815, WO 1999/32646, WO 2001/79518, WO 2003/064665, U.S. Pat. No. 6,969,598, WO2019028051, WO2016100232A1, WO2018067992A, each of which is incorporated by reference in its entirety.

In some embodiments, the nucleic acid molecule is a DNA vector. In some embodiments, the DNA vector is chosen from Table 13, and, within a multiple cloning site, the one or more of the disclosed nucleic acid sequences encoding a first CAR, a second CAR or both a first and second CAR are positioned in an open reading frame for expression of the first CAR, second CAR or both the first and second CAR.

TABLE 13
Vector Backbone Sequences

| SEQ | | |
|---|---|---|
| ID | | |
| NO: | Name: | Sequence: |
| 796 | pAN2414 | TTACTCTAAACCTGTGATTCCTCTGAATTATTTTCATTTTAAAGAAATTGTAT |
| | | TTGTTAAATATGTACTACAAACTTAGTAGTTGGAAGGGCTAATTCACTCCC |
| | | AAAGAAGACAAGATATCCTTGATCTGTGGATCTACCACACACAAGGCTAC |
| | | TTCCCTGATTAGCAGAACTACACACCAGGGCCAGGGGTCAGATATCCACT |
| | | GACCTTTGGATGGTGCTACAAGCTAGTACCAGTTGAGCCAGATAAGGTAG |
| | | AAGAGGCCAATAAAGGAGAGAACACCAGCTTGTTACACCCTGTGAGCCT |
| | | GCATGGGATGGATGACCCGGAGAGAGAAGTGTTAGAGTGGAGGTTTGAC |
| | | AGCCGCCTAGCATTTCATCACGTGGCCCGAGAGCTGCATCCGGAGTACTTC |
| | | AAGAACTGCTGATATCGAGCTTGCTACAAGGGACTTTCCGCTGGGGACTT |
| | | TCCAGGGAGGCGTGGCCTGGGCGGGACTGGGGAGTGGCGAGCCCTCAGA |
| | | TCCTGCATATAAGCAGCTGCTTTTTGCCTGTACTGGGACTCTCTGGTTAGA |
| | | CCAGATCTGAGCCTGGGAGCTCTCTGGCTAACTAGGGAACCCACTGCTTA |
| | | AGCCTCAATAAAGCTTGCCTTGAGTGCTTCAAGTAGTGTGTGCCCGTCTGT |
| | | TGTGTGACTCTGGTAACTAGAGATCCCTCAGACCCTTTTAGTCAGTGTGGA |
| | | AAATCTCTAGCAGTGGCGCCCGAACAGGGACTTGAAAGCGAAAGGGAAA |
| | | CCAGAGGAGCTCTCTCGACGCAGGACTCGGCTTGCTGAAGCGCGCACGG |
| | | CAAGAGGCGAGGGGCGGCGACTGGTGAGTACGCCAAAAATTTTGACTAG |
| | | CGGAGGCTAGAAGGAGAGAGATGGGTGCGAGAGCGTCAGTATTAAGCGG |
| | | GGGAGAATTAGATCGCGATGGGAAAAAATTCGGTTAAGGCCAGGGGGAA |
| | | AGAAAAAATATAAATTAAAACATATAGTATGGGCAAGCAGGGAGCTAGAA |
| | | CGATTCGCAGTTAATCCTGGCCTGTTAGAAACATCAGAAGGCTGTAGACA |
| | | AATACTGGGACAGCTACAACCATCCCTTCAGACAGGATCAGAAGAACTTA |
| | | GATCATTATATAATACAGTAGCAACCCTCTATTGTGTGCATCAAAGGATAGA |
| | | GATAAAAGACACCAAGGAAGCTTTAGACAAGATAGAGGAAGAGCAAAAC |
| | | AAAAGTAAGACCACCGCACAGCAAGCGGCCGGCCGCTGATCTTCAGACC |
| | | TGGAGGAGGAGATATGAGGGACAATTGGAGAAGTGAATTATATAAATATAA |
| | | AGTAGTAAAAATTGAACCATTAGGAGTAGCACCCACCAAGGCAAAGAGA |
| | | AGAGTGGTGCAGAGAGAAAAAAGAGCAGTGGGAATAGGAGCTTTGTTCC |
| | | TTGGGTTCTTGGGAGCAGCAGGAAGCACTATGGGCGCAGCGTCAATGACG |
| | | CTGACGGTACAGGCCAGACAATTATTGTCTGGTATAGTGCAGCAGCAGAA |
| | | CAATTTGCTGAGGGCTATTGAGGCGCAACAGCATCTGTTGCAACTCACAG |
| | | TCTGGGGCATCAAGCAGCTCCAGGCAAGAATCCTGGCTGTGGAAAGATAC |
| | | CTAAAGGATCAACAGCTCCTGGGGATTTGGGGTTGCTCTGGAAAACTCAT |
| | | TTGCACCACTGCTGTGCCTTGGAATGCTAGTTGGAGTAATAAATCTCTGGA |
| | | ACAGATTTGGAATCACACGACCTGGATGGAGTGGGACAGAGAAATTAACA |
| | | ATTACACAAGCTTAATACACTCCTTAATTGAAGAATCGCAAAACCAGCAAG |
| | | AAAAGAATGAACAAGAATTATTGGAATTAGATAAATGGGCAAGTTTGTGG |
| | | AATTGGTTTAACATAACAAATTGGCTGTGGTATATAAAATTATTCATAATGAT |
| | | AGTAGGAGGCTTGGTAGGTTTAAGAATAGTTTTTGCTGTACTTTCTATAGTG |
| | | AATAGAGTTAGGCAGGGATATTCACCATTATCGTTTCAGACCCACCTCCCA |
| | | ACCCCGAGGGGACCCGACAGGCCCGAAGGAATAGAAGAAGAAGGTGGA |
| | | GAGAGAGACAGAGACAGATCCATTCGATTAGTGAACGGATCTCGACGGTA |
| | | TCGCCAAATGGCAGTATTCATCCACAATTTTAAAAGAAAAGGGGGGATTG |
| | | GGGGGTACAGTGCAGGGGAAAGAATAGTAGACATAATAGCAACAGACATA |
| | | CAAACTAAAGAATTACAAAAACAAATTACAAAAATTCAAAATTTTCGGGT |
| | | TTATTACAGGGACAGCAGAGATCCAGTTTGGCCCTCTGATGAGACGAACG |
| | | TGAGACCGAAAGTGAAACGTGATTTCATGCGTCATTTTGAACATTTTGTAA |
| | | ATCTTATTTAATAATGTGTGCGGCAATTCACATTTAATTTATGAATGTTTTCT |
| | | TAACATCGCGGCAACTCAAGAAACGGCAGGTTCGGATCTTAGCTACTAGA |
| | | GAAAGAGGAGAAATACTAGATGCGTAAAGGCGAAGAGCTGTTCACTGGT |
| | | GTCGTCCCTATTCTGGTGGAACTGGATGGTGATGTCAACGGTCATAAGTTT |
| | | TCCGTGCGTGGCGAGGGTGAAGGTGACGCAACTAATGGTAAACTGACGCT |
| | | GAAGTTCATCTGTACTACTGGTAAACTGCCGGTTCCTTGGCCGACTCTGGT |
| | | AACGACGCTGACTTATGGTGTTCAGTGCTTTGCTCGTTATCCGGACCATAT |
| | | GAAGCAGCATGACTTCTTCAAGTCCGCCATGCCGGAAGGCTATGTGCAGG |
| | | AACGCACGATTTCCTTTAAGGATGACGGCACGTACAAAACGCGTGCGGAA |
| | | GTGAAATTTGAAGGCGATACCCTGGTAAACCGCATTGAGCTGAAAGGCAT |
| | | TGACTTTAAAGAGGACGGCAATATCCTGGGCCATAAGCTGGAATACAATTT |
| | | TAACAGCCACAATGTTTACATCACCGCCGATAAACAAAAAAATGGCATTAA |
| | | AGCGAATTTTAAAATTCGCCACAACGTGGAGGATGGCAGCGTGCAGCTGG |
| | | CTGATCACTACCAGCAAAACACTCCAATCGGTGATGGTCCTGTTCTGCTGC |
| | | CAGACAATCACTATCTGAGCACGCAAAGCGTTCTGTCTAAAGATCCGAAC |
| | | GAGAAACGCGATCATATGGTTCTGCTGGAGTTCGTAACCGCAGCGGGCAT |
| | | CACGCATGGTATGGATGAACTGTACAAATGACCAGGCATCAAATAAAACG |
| | | AAAGGCTCAGTCGAAAGACTGGGCCTTTCGTTTTATCTGTTGTTTGTCGGT |
| | | GAACGCTCTCTACTAGAGTCACACTGGCTCACCTTCGGGTGGGCCTTTCTG |
| | | CGTTTATAGGTCTCAGCTGCGTCTCAAGCATACACGATACCGTCGACCTCG |
| | | AGGGAATTAATTCGAGCTCGGTACCTTTAAGACCAATGACTTACAAGGCA |
| | | GCTGTAGATCTTAGCCACTTTTTAAAAGAAAAGGGGGGACTGGAAGGGCT |
| | | AATTCACTCCCAACGAAGACAAGATCTGCTTTTTGCTTGTACTGGGTTTCT |
| | | CTGGTTAGACCAGATCTGAGCCTGGGAGCTCTCTGGCTAACTAGGGAACC |
| | | CACTGCTTAAGCCTCAATAAAGCTTGCCTTGAGTGCTTCAAGTAGTGTGTG |
| | | CCCGTCTGTTGTGTGACTCTGGTAACTAGAGATCCCTCAGACCCTTTTAGT |
| | | CAGTGTGGAAAATCTCTAGCAGCATCTAGAATTAATTCCGTGTATTCTATAG |
| | | TGTCACCTAAATCGTATGTGTATGATACATAAGGTTATGTATTAATTGTAGCC |
| | | GCGTTCTAACGACAATATGTACAAGCCTAATTGTGTAGCATCTGGCTTACTG |
| | | AAGCAGACCCTATCATCTCTCTCGTAAACTGCCGTCAGAGTCGGTTTGGTT |
| | | GGACGAACCTTCTGAGTTTCTGGTAACGCCGTCCCGCACCCGGAAATGGT |
| | | CAGCGAACCAATCAGCAGGGTCATCGCTAGCCAGATCCTCTACGCCGGAC |
| | | GCATCGTGGCCGGCATCACCGGCGCCACAGGTGCGGTTGCTGGCGCCTAT |
| | | ATCGCCGACATCACCGATGGGGAAGATCGGGCTCGCCACTTCGGGCTCAT |
| | | GAGCGCTTGTTTCGGCGTGGGTATGGTGGCAGGCCCCGTGGCCGGGGGAC |
| | | TGTTGGGCGCCATCTCCTTGCATGCACCATTCCTTGCGGCGGCGGTGCTCA |
| | | ACGGCCTCAACCTACTACTGGGCTGCTTCCTAATGCAGGAGTCGCATAAGG |
| | | GAGAGCGTCGAATGGTGCACTCTCAGTACAATCTGCTCTGATGCCGCATAG |
| | | TTAAGCCAGCCCCGACACCCGCCAACACCCGCTGACGCGCCCTGACGGG |
| | | CTTGTCTGCTCCCGGCATCCGCTTACAGACAAGCTGTGACCATCTCCGGGA |
| | | GCTGCATGTGTCAGAGGTTTTCACCGTCATCACCGAAACGCGCGAGATGA |
| | | AAGGGCCTCGTGATACGCCTATTTTTATAGGTTAATGTCATGATAATAATGG |
| | | TTTCTTAGACGTCAGGTGGCACTTTTCGGGGAAATGTGCCGAGCGGCCGC |
| | | ACGGTTATCCACAGAATCAGGGGATAACGCAGGAAAGAACATGTGAGCAA |
| | | AAGGCCAGCAAAAGGCCAGGAACCGTAAAAAGGCCGCGTTGCTGGCGTT |
| | | TTTCCATAGGCTCCGCCCCCCTGACGAGCATCACAAAAATCGACGCTCAA |
| | | GTCAGAGGTGGCGAAACCCGACAGGACTATAAAGATACCAGGCGTTTCCC |
| | | CCTGGAAGCTCCCTCGTGCGCTCTCCTGTTCCGACCCTGCCGCTTACCGGA |
| | | TACCTGTCCGCCTTTCTCCCTTCGGGAAGCGTGGCGCTTTCTCATAGCTCA |
| | | CGCTGTAGGTATCTCAGTTCGGTGTAGGTCGTTCGCTCCAAGCTGGGCTGT |
| | | GTGCACGAACCCCCCGTTCAGCCCGACCGCTGCGCCTTATCCGGTAACTAT |
| | | CGTCTTGAGTCCAACCCGGTAAGACACGACTTATCGCCACTGGCAGCAGC |
| | | CACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGGTA |
| | | TCTGCGCTCTGCTGAAGCCAGTTACCTTCGGAAAAAGAGTTGGTAGCTCT |
| | | TGATCCGGCAAACAAACCACCGCTGGTAGCGGTGGTTTTTTTGTTTGCAA |
| | | GCAGCAGATTACGCGCAGAAAAAAAGGATCTCAAGAAGATCCTTTGATCT |
| | | TTTCTACGGGGTCTGACGCTCAGTGGAACGAAAACTCACGTTAAGGGATT |
| | | TTGGTCATGACAGGTGGCACTTTTCGGGGAAATGTGCGCGGAACCCCTATT |
| | | TGTTTATTTTTCTAAATACATTCAAATATGTATCCGCTCATGAGACAATAACC |
| | | CTGATAAATGCTTCAATAATATTGAAAAAGGAAGAGTATGAGTATTCAACA |
| | | TTTCCGTGTCGCCCTTATTCCCTTTTTTGCGGCATTTTGCCTTCCTGTTTTTG |
| | | CTCACCCAGAAACGCTGGTGAAAGTAAAAGATGCTGAAGATCAGTTGGGT |
| | | GCACGAGTGGGTTACATCGAACTGGATCTCAACAGCGGTAAGATCCTTGA |
| | | GAGTTTTCGCCCCGAAGAACGTTTTCCAATGATGAGCACTTTTAAAGTTCT |
| | | GCTATGTGGCGCGGTATTATCCCGTATTGACGCCGGGCAAGAGCAACTCGG |
| | | TCGCCGCATACACTATTCTCAGAATGACTTGGTTGAGTACTCACCAGTCAC |
| | | AGAAAAGCATCTTACGGATGGCATGACAGTAAGAGAATTATGCAGTGCTG |
| | | CCATAACCATGAGTGATAACACTGCGGCCAACTTACTTCTGACAACGATCG |
| | | GAGGACCGAAGGAGCTAACCGCTTTTTTGCACAACATGGGGGATCATGTA |
| | | ACTCGCCTTGATCGTTGGGAACCGGAGCTGAATGAAGCCATACCAAACGA |
| | | CGAGCGTGACACCACGATGCCTGTAGCAATGGCAACAACGTTGCGCAAAC |
| | | TATTAACTGGCGAACTACTTACTCTAGCTTCCCGGCAACAATTAATAGACT |
| | | GGATGGAGGCGGATAAAGTTGCAGGACCACTTCTGCGCTCGGCCCTTCCG |
| | | GCTGGCTGGTTTATTGCTGATAAATCTGGAGCCGGTGAGCGTGGGTCCCGC |
| | | GGTATCATTGCAGCACTGGGGCCAGATGGTAAGCCCTCCCGTATCGTAGTT |
| | | ATCTACACGACGGGGAGTCAGGCAACTATGGATGAACGAAATAGACAGAT |
| | | CGCTGAGATAGGTGCCTCACTGATTAAGCATTGGTAACTGTCAGACCAAGT |
| | | TTACTCATATATACTTTAGATTGATTTAAAACTTCATTTTTAATTTAAAAGGA |
| | | TCTAGGTGAAGATCCTTTTTGATAATCGCGGCCGCCAATTTTTTACGGTTCC |
| | | TGGCCTTTTGCTGGCCTTTTGCTCACATGTTCTTTCCTGCGTTATCCCCTGA |
| | | TTCTGTGGATAACCGTATTACCGCCTTTGAGTGAGCTGATACCGCTCGCCG |
| | | CAGCCGAACGACCGAGCGCAGCGAGTCAGTGAGCGAGGAAGCGGAAGA |
| | | GCGCCCAATACGCAAACCGCCTCTCCCCGCGCGTTGGCCGATTCATTAATG |
| | | CAGCTGTGGAATGTGTGTCAGTTAGGGTGTGGAAAGTCCCCAGGCTCCCC |
| | | AGCAGGCAGAAGTATGCAAAGCATGCATCTCAATTAGTCAGCAACCAGGT |
| | | GTGGAAAGTCCCCAGGCTCCCCAGCAGGCAGAAGTATGCAAAGCATGCAT |
| | | CTCAATTAGTCAGCAACCATAGTCCCGCCCCTAACTCCGCCCATCCCGCCC |
| | | CTAACTCCGCCCAGTTCCGCCCATTCTCCGCCCCATGGCTGACTAATTTTTT |
| | | TTATTTATGCAGAGGCCGAGGCCGCCTCGGCCTCTGAGCTATTCCAGAAGT |
| | | AGTGAGGAGGCTTTTTTGGAGGCCTAGGCTTTTGCAAAAAGCTTGGACAC |
| | | AAGACAGGCTTGCGAGATATGTTTGAGAATACCACTTTATCCCGCGTCAGG |
| | | GAGAGGCAGTGCGTAAAAAGACGCGGACTCATGTGAAATACTGGTTTTTA |
| | | GTGCGCCAGATCTCTATAATCTCGCGCAACCTATTTTCCCCTCGAACACTTT |
| | | TTAAGCCGTAGATAAACAGGCTGGGACACTTCACATGAGCGAAAAATACA |
| | | TCGTCACCTGGGACATGTTGCAGATCCATGCACGTAAACTCGCAAGCCGA |
| | | CTGATGCCTTCTGAACAATGGAAAGGCATTATTGCCGTAAGCCGTGGCGGT |
| | | CTGTACCGGGTGCGTTACTGGCGCGTGAACTGGGTATTCGTCATGTCGATA |
| | | CCGTTTGTATTTCCAGCTACGATCACGACAACCAGCGCGAGCTTAAAGTGC |
| | | TGAAACGCGCAGAAGGCGATGGCGAAGGCTTCATCGTTATTGATGACCTG |
| | | GTGGATACCGGTGGTACTGCGGTTGCGATTCGTGAAATGTATCCAAAAGCG |
| | | CACTTTGTCACCATCTTCGCAAAACCGGCTGGTCGTCCGCTGGTTGATGAC |
| | | TATGTTGTTGATATCCCGCAAGATACCTGGATTGAACAGCCGTGGGATATG |
| | | GGCGTCGTATTCGTCCCGCCAATCTCCGGTCGCTAATCTTTTCAACGCCTG |
| | | GCACTGCCGGGCGTTGTTCTTTTTAACTTCAGGCGGGTTACAATAGTTTCC |
| | | AGTAAGTATTCTGGAGGCTGCATCCATGACACAGGCAAACCTGAGCGAAA |
| | | CCCTGTTCAAACCCCGCTTTAAACATCCTGAAACCTCGACGCTAGTCCGCC |
| | | GCTTTAATCACGGCGCACAACCGCCTGTGCAGTCGGCCCTTGATGGTAAA |
| | | ACCATCCCTCACTGGTATCGCATGATTAACCGTCTGATGTGGATCTGGCGC |
| | | GGCATTGACCCACGCGAAATCCTCGACGTCCAGGCACGTATTGTGATGAG |
| | | CGATGCCGAACGTACCGACGATGATTTATACGATACGGTGATTGGCTACCG |
| | | TGGCGGCAACTGGATTTATGAGTGGGCCCCGGATCTTTGTGAAGGAACCT |
| | | TACTTCTGTGGTGTGACATAATTGGACAAACTACCTACAGAGATTTAAAGC |
| | | TCTAAGGTAAATATAAAATTTTTAAGTGTATAATGTGTTAAACTACTGATTCT |
| | | AATTGTTTGTGTATTTTAGATTCCAACCTATGGAACTGATGAATGGGAGCA |
| | | GTGGTGGAATGCCTTTAATGAGGAAAACCTGTTTTGCTCAGAAGAAATGC |
| | | CATCTAGTGATGATGAGGCTACTGCTGACTCTCAACATTCTACTCCTCCAA |
| | | AAAAGAAGAGAAAGGTAGAAGACCCCAAGGACTTTCCTTCAGAATTGCT |
| | | AAGTTTTTTGAGTCATGCTGTGTTTAGTAATAGAACTCTTGCTTGCTTTGCT |
| | | ATTTACACCACAAAGGAAAAAGCTGCACTGCTATACAAGAAAATTATGGA |
| | | AAAATATTCTGTAACCTTTATAAGTAGGCATAACAGTTATAATCATAACATAC |
| | | TGTTTTTTCTTACTCCACACAGGCATAGAGTGTCTGCTATTAATAACTATGC |
| | | TCAAAAATTGTGTACCTTTAGCTTTTTAATTTGTAAAGGGGTTAATAAGGA |
| | | ATATTTGATGTATAGTGCCTTGACTAGAGATCATAATCAGCCATACCACATTT |
| | | GTAGAGGTTTTACTTGCTTTAAAAAACCTCCCACACCTCCCCCTGAACCTG |
| | | AAACATAAAATGAATGCAATTGTTGTTGTTAACTTGTTTATTGCAGCTTATA |
| | | ATGGTTACAAATAAAGCAATAGCATCACAAATTTCACAAATAAAGCATTTT |
| | | TTTCACTGCATTCTAGTTGTGGTTTGTCCAAACTCATCAATGTATCTTATCAT |
| | | GTCTGGATCAACTGGATAACTCAAGCTAACCAAAATCATCCCAAACTTCCC |
| | | ACCCCATACCCTATTACCACTGCCAATTACCTGTGGTTTCAT |
| 797 | CaTCH_empty- | AGAGTTAGGCAGGGATATTCACCATTATCGTTTCAGACCCACCTCCCAACC |
| | backbone | CCGAGGGGACCCTATCATATGCTGGGTACCCTGTCTAGAGCGAACGGAGA |
| | | CGACTAGTGATATCCGTCTCTCGGCAGGAGAAGCTTTAGGCGTGTACGGTG |
| | | GGCGCCTATAAAAGCAGAGCTCGTTTAGTGAACCGTCAGATCGCCTGGAG |
| | | CAATTCCACAACACTTTTGTCTTATACCAACTTTCCGTACCACTTCCTACCC |
| | | TCGTAAACTGGAGGATCCGCCACCATGGTGAGCAAGGGCGAGGAGCTGTT |
| | | CACCGGGGTGGTGCCCATCCTGGTCGAGCTGGACGGCGACGTAAACGGC |
| | | CACAAGTTCAGCGTGTCCGGCGAGGGCGAGGGCGATGCCACCTACGGCA |
| | | AGCTGACCCTGAAGTTCATCTGCACCACCGGCAAGCTGCCCGTGCCCTGG |
| | | CCCACCCTCGTGACCACCCTGACCTACGGCGTGCAGTGCTTCAGCCGCTA |
| | | CCCCGACCACATGAAGCAGCACGACTTCTTCAAGTCCGCCATGCCCGAAG |
| | | GCTACGTCCAGGAGCGCACCATCTTCTTCAAGGACGACGGCAACTACAAG |
| | | ACCCGCGCCGAGGTGAAGTTCGAGGGCGACACCCTGGTGAACCGCATCG |
| | | AGCTGAAGGGCATCGACTTCAAGGAGGACGGCAACATCCTGGGGCACAA |
| | | GCTGGAGTACAACTACAACAGCCACAACGTCTATATCATGGCCGACAAGC |
| | | AGAAGAACGGCATCAAGGTGAACTTCAAGATCCGCCACAACATCGAGGA |
| | | CGGCAGCGTGCAGCTCGCCGACCACTACCAGCAGAACACCCCCATCGGC |
| | | GACGGCCCCGTGCTGCTGCCCGACAACCACTACCTGAGCACCCAGTCCGC |
| | | CCTGAGCAAAGACCCCAACGAGAAGCGCGATCACATGGTCCTGCTGGAG |
| | | TTCGTGACCGCCGCCGGGATCACTCTCGGCATGGACGAGCTGTACAAGTA |
| | | AGAATTCTAGATCTTGAGACAAATGGCAGTATTCATCCACAATTTTAAAAG |
| | | AAAAGGGGGGATTGGGGGGTACAGTGCAGGGGAAAGAATAGTAGACATA |
| | | ATAGCAACAGACATACAAACTAAAGAATTACAAAAACAAATTACAAAAAT |
| | | TCAAAATTTTCGGGTTTATTACAGGGACAGCAGAGATCCACTTTGGCGCCG |
| | | GCTCGAGTGGCTCCGGTGCCCGTCAGTGGGCAGAGCGCACATCGCCCACA |
| | | GTCCCCGAGAAGTTGGGGGGAGGGGTCGGCAATTGAACCGGTGCCTAGA |
| | | GAAGGTGGCGCGGGGTAAACTGGGAAAGTGATGTCGTGTACTGGCTCCGC |
| | | CTTTTTCCCGAGGGTGGGGGAGAACCGTATATAAGTGCAGTAGTCGCCGT |
| | | GAACGTTCTTTTTCGCAACGGGTTTGCCGCCAGAACACAGGTGTCGTGAC |
| | | GCGGATCGTACCCCGAATTTATGCTCATGGTGAGCAAGGGCGAGGAGCTG |
| | | TTCACCGGGGTGGTGCCCATCCTGGTCGAGCTGGACGGCGACGTAAACGG |
| | | CCACAAGTTCAGCGTGAGGGGCGAGGGCGAGGGCGATGCCACCAACGGC |
| | | AAGCTGACCCTGAAGTTCATCTGCACCACCGGCAAGCTGCCCGTGCCCTG |
| | | GCCCACCCTCGTGACCACCCTGAGCCACGGCGTGCAGTGCTTCGCCCGCT |
| | | ACCCCGACCACATGAAGCAGCACGACTTCTTCAAGTCCGCCATGCCCGAA |
| | | GGCTACGTCCAGGAGCGCACCATCTTCTTCAAGGACGACGGCACCTACAA |
| | | GACCCGCGCCGAGGTGAAGTTCGAGGGCGACACCCTGGTGAACCGCATC |
| | | GAGCTGAAGGGCGTCGACTTCAAGGAGGACGGCAACATCCTGGGGCACA |
| | | AGCTGGAGTACAACTTCAACAGCCACAACATCTATATCATGGCCGTCAAGC |
| | | AGAAGAACGGCATCAAGGTGAACTTCAAGATCCGCCACAACGTGGAGGA |
| | | CGGCAGCGTGCAGCTCGCCGACCACTACCAGCAGAACACCCCCATCGGC |
| | | GACGGCCCCGTGCTGCTGCCCGACAGCCACTACCTGAGCACCCAGTCCGT |
| | | GCTGAGCAAAGACCCCAACGAGAAGCGCGATCACATGGTCCTGCTGGAG |
| | | TTCCGCACCGCCGCCGGGATCACTCTCGGCATGGACGAGCTGTACAAGTA |
| | | ACGCGTTAAGTCGACAATCAACCTCTGGATTACAAAATTTGTGAAAGATTG |
| | | ACTGGTATTCTTAACTATGTTGCTCCTTTTACGCTATGTGGATACGCTGCTTT |
| | | AATGCCTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGT |
| | | TGGGGCATTGCCACCACCTGTCAGCTCCTTTCCGGGACTTTCGCTTTCCCC |
| | | CTCCCTATTGCCACGGCGGAACTCATCGCCGCCTGCCTTGCCCGCTGCTGG |
| | | ACAGGGGCTCGGCTGTTGGGCACTGACAATTCCGTGGTGTTGTCGGGGAA |
| | | ATCATCGTCCTTTCCTTGGCTGCTCGCCTGTGTTGCCACCTGGATTCTGCGC |
| | | GGGACGTCCTTCTGCTACGTCCCTTCGGCCCTCAATCCAGCGGACCTTCCT |
| | | TCCCGCGGCCTGCTGCCGGCTCTGCGGCCTCTTCCGCGTCTTCGCCTTCGC |
| | | CCTCAGACGAGTCGGATCTCCCTTTGGGCCGCCTCCCCGCGTCGACTTTAA |
| | | GACCAATGACTTACAAGGCAGCTGTAGATCTTAGCCACTTTTTAAAAGAA |
| | | AAGGGGGGACTGGAAGGGCTAATTCACTCCCAACGAAGACAAGATCTGC |
| | | TTTTTGCTTGTACTGGGTCTCTCTGGTTAGACCAGATCTGAGCCTGGGAGC |
| | | TCTCTGGCTAACTAGGGAACCCACTGCTTAAGCCTCAATAAAGCTTGCCTT |
| | | GAGTGCTTCAAGTAGTGTGTGCCCGTCTGTTGTGTGACTCTGGTAACTAGA |
| | | GATCCCTCAGACCCTTTTAGTCAGTGTGGAAAATCTCTAGCAGTACGTATA |
| | | GTAGTTCATGTCATCTTATTATTCAGTATTTATAACTTGCAAAGAAATGAATA |
| | | TCAGAGAGTGAGAGGAACTTGTTTATTGCAGCTTATAATGGTTACAAATAA |
| | | AGCAATAGCATCACAAATTTCACAAATAAAGCATTTTTTTCACTGCATTCTA |
| | | GTTGTGGTTTGTCCAAACTCATCAATGTATCTTATCATGTCTGGCTCTAGCT |
| | | ATCCCGCCCCTAACTCCGCCCATCCCGCCCCTAACTCCGCCCAGTTCCGCC |
| | | CATTCTCCGCCCCATGGCTGACTAATTTTTTTTATTTATGCAGAGGCCGAGG |
| | | CCGCCTCGGCCTCTGAGCTATTCCAGAAGTAGTGAGGAGGCTTTTTTGGA |
| | | GGCCTAGGGACGTACCCAATTCGCCCTATAGTGAGTCGTATTACGCGCGCT |
| | | CACTGGCCGTCGTTTTACAACGTCGTGACTGGGAAAACCCTGGCGTTACC |
| | | CAACTTAATCGCCTTGCAGCACATCCCCCTTTCGCCAGCTGGCGTAATAGC |
| | | GAAGAGGCCCGCACCGATCGCCCTTCCCAACAGTTGCGCAGCCTGAATGG |
| | | CGAATGGGACGCGCCCTGTAGCGGCGCATTAAGCGCGGCGGGTGTGGTGG |
| | | TTACGCGCAGCGTGACCGCTACACTTGCCAGCGCCCTAGCGCCCGCTCCT |
| | | TTCGCTTTCTTCCCTTCCTTTCTCGCCACGTTCGCCGGCTTTCCCCGTCAAG |
| | | CTCTAAATCGGGGGCTCCCTTTAGGGTTCCGATTTAGTGCTTTACGGCACC |
| | | TCGACCCCAAAAAACTTGATTAGGGTGATGGTTCACGTAGTGGGCCATCG |
| | | CCCTGATAGACGGTTTTTCGCCCTTTGACGTTGGAGTCCACGTTCTTTAAT |
| | | AGTGGACTCTTGTTCCAAACTGGAACAACACTCAACCCTATCTCGGTCTAT |
| | | TCTTTTGATTTATAAGGGATTTTGCCGATTTCGGCCTATTGGTTAAAAAATG |
| | | AGCTGATTTAACAAAAATTTAACGCGAATTTTAACAAAATATTAACGCTTA |
| | | CAATTTAGGTGGCACTTTTCGGGGAAATGTGCGCGGAACCCCTATTTGTTT |
| | | ATTTTTCTAAATACATTCAAATATGTATCCGCTCATGAGACAATAACCCTGA |
| | | TAAATGCTTCAATAATATTGAAAAAGGAAGAGTATGAGTATTCAACATTTC |
| | | CGTGTCGCCCTTATTCCCTTTTTTGCGGCATTTTGCCTTCCTGTTTTTGCTC |
| | | ACCCAGAAACGCTGGTGAAAGTAAAAGATGCTGAAGATCAGTTGGGTGC |
| | | ACGAGTGGGTTACATCGAACTGGATCTCAACAGCGGTAAGATCCTTGAGA |
| | | GTTTTCGCCCCGAAGAACGTTTTCCAATGATGAGCACTTTTAAAGTTCTGC |
| | | TATGTGGCGCGGTATTATCCCGTATTGACGCCGGGCAAGAGCAACTCGGTC |
| | | GCCGCATACACTATTCTCAGAATGACTTGGTTGAGTACTCACCAGTCACAG |
| | | AAAAGCATCTTACGGATGGCATGACAGTAAGAGAATTATGCAGTGCTGCC |
| | | ATAACCATGAGTGATAACACTGCGGCCAACTTACTTCTGACAACGATCGGA |
| | | GGACCGAAGGAGCTAACCGCTTTTTTGCACAACATGGGGGATCATGTAAC |
| | | TCGCCTTGATCGTTGGGAACCGGAGCTGAATGAAGCCATACCAAACGACG |
| | | AGCGTGACACCACGATGCCTGTAGCAATGGCAACAACGTTGCGCAAACTA |
| | | TTAACTGGCGAACTACTTACTCTAGCTTCCCGGCAACAATTAATAGACTGG |
| | | ATGGAGGCGGATAAAGTTGCAGGACCACTTCTGCGCTCGGCCCTTCCGGC |
| | | TGGCTGGTTTATTGCTGATAAATCTGGAGCCGGTGAGCGTGGGTCTCGCGG |
| | | TATCATTGCAGCACTGGGGCCAGATGGTAAGCCCTCCCGTATCGTAGTTAT |
| | | CTACACGACGGGGAGTCAGGCAACTATGGATGAACGAAATAGACAGATCG |
| | | CTGAGATAGGTGCCTCACTGATTAAGCATTGGTAACTGTCAGACCAAGTTT |
| | | ACTCATATATACTTTAGATTGATTTAAAACTTCATTTTTAATTTAAAAGGATC |
| | | TAGGTGAAGATCCTTTTTGATAATCTCATGACCAAAATCCCTTAACGTGAG |
| | | TTTTCGTTCCACTGAGCGTCAGACCCCGTAGAAAAGATCAAAGGATCTTCT |
| | | TGAGATCCTTTTTTTCTGCGCGTAATCTGCTGCTTGCAAACAAAAAAACCA |
| | | CCGCTACCAGCGGTGGTTTGTTTGCCGGATCAAGAGCTACCAACTCTTTTT |
| | | CCGAAGGTAACTGGCTTCAGCAGAGCGCAGATACCAAATACTGTTCTTCTA |
| | | GTGTAGCCGTAGTTAGGCCACCACTTCAAGAACTCTGTAGCACCGCCTAC |
| | | ATACCTCGCTCTGCTAATCCTGTTACCAGTGGCTGCTGCCAGTGGCGATAA |
| | | GTCGTGTCTTACCGGGTTGGACTCAAGACGATAGTTACCGGATAAGGCGC |
| | | AGCGGTCGGGCTGAACGGGGGGTTCGTGCACACAGCCCAGCTTGGAGCG |
| | | AACGACCTACACCGAACTGAGATACCTACAGCGTGAGCTATGAGAAAGCG |
| | | CCACGCTTCCCGAAGGGAGAAAGGCGGACAGGTATCCGGTAAGCGGCAG |
| | | GGTCGGAACAGGAGAGCGCACGAGGGAGCTTCCAGGGGGAAACGCCTG |
| | | GTATCTTTATAGTCCTGTCGGGTTTCGCCACCTCTGACTTGAGCGTCGATTT |
| | | TTGTGATGCTCGTCAGGGGGGCGGAGCCTATGGAAAAACGCCAGCAACGC |
| | | GGCCTTTTTACGGTTCCTGGCCTTTTGCTGGCCTTTTGCTCACATGTTCTTT |
| | | CCTGCGTTATCCCCTGATTCTGTGGATAACCGTATTACCGCCTTTGAGTGAG |
| | | CTGATACCGCTCGCCGCAGCCGAACGACCGAGCGCAGCGAGTCAGTGAG |
| | | CGAGGAAGCGGAAGAGCGCCCAATACGCAAACCGCCTCTCCCCGCGCGT |
| | | TGGCCGATTCATTAATGCAGCTGGCACGACAGGTTTCCCGACTGGAAAGC |
| | | GGGCAGTGAGCGCAACGCAATTAATGTGAGTTAGCTCACTCATTAGGCAC |
| | | CCCAGGCTTTACACTTTATGCTTCCGGCTCGTATGTTGTGTGGAATTGTGA |
| | | GCGGATAACAATTTCACACAGGAAACAGCTATGACCATGATTACGCCAAG |
| | | CGCGCAATTAACCCTCACTAAAGGGAACAAAAGCTGGAGCTGCAAGCTTA |
| | | ATGTAGTCTTATGCAATACTCTTGTAGTCTTGCAACATGGTAACGATGAGTT |
| | | AGCAACATGCCTTACAAGGAGAGAAAAAGCACCGTGCATGCCGATTGGTG |
| | | GAAGTAAGGTGGTACGATCGTGCCTTATTAGGAAGGCAACAGACGGGTCT |
| | | GACATGGATTGGACGAACCACTGAATTGCCGCATTGCAGAGATATTGTATT |
| | | TAAGTGCCTAGCTCGATACATAAACGGGTCTCTCTGGTTAGACCAGATCTG |
| | | AGCCTGGGAGCTCTCTGGCTAACTAGGGAACCCACTGCTTAAGCCTCAAT |
| | | AAAGCTTGCCTTGAGTGCTTCAAGTAGTGTGTGCCCGTCTGTTGTGTGACT |
| | | CTGGTAACTAGAGATCCCTCAGACCCTTTTAGTCAGTGTGGAAAATCTCTA |
| | | GCAGTGGCGCCCGAACAGGGACTTGAAAGCGAAAGGGAAACCAGAGGA |
| | | GCTCTCTCGACGCAGGACTCGGCTTGCTGAAGCGCGCACGGCAAGAGGC |
| | | GAGGGGCGGCGACTGGTGAGTACGCCAAAAATTTTGACTAGCGGAGGCT |
| | | AGAAGGAGAGAGATGGGTGCGAGAGCGTCAGTATTAAGCGGGGGAGAAT |
| | | TAGATCGCGATGGGAAAAAATTCGGTTAAGGCCAGGGGGAAAGAAAAAA |
| | | TATAAATTAAAACATATAGTATGGGCAAGCAGGGAGCTAGAACGATTCGCA |
| | | GTTAATCCTGGCCTGTTAGAAACATCAGAAGGCTGTAGACAAATACTGGG |
| | | ACAGCTACAACCATCCCTTCAGACAGGATCAGAAGAACTTAGATCATTATA |
| | | TAATACAGTAGCAACCCTCTATTGTGTGCATCAAAGGATAGAGATAAAAGA |
| | | CACCAAGGAAGCTTTAGACAAGATAGAGGAAGAGCAAAACAAAAGTAAG |
| | | ACCACCGCACAGCAAGCGGCCGCTGATCTTCAGACCTGGAGGAGGAGAT |
| | | ATGAGGGACAATTGGAGAAGTGAATTATATAAATATAAAGTAGTAAAAATT |
| | | GAACCATTAGGAGTAGCACCCACCAAGGCAAAGAGAAGAGTGGTGCAGA |
| | | GAGAAAAAAGAGCAGTGGGAATAGGAGCTTTGTTCCTTGGGTTCTTGGGA |
| | | GCAGCAGGAAGCACTATGGGCGCAGCGTCAATGACGCTGACGGTACAGG |
| | | CCAGACAATTATTGTCTGGTATAGTGCAGCAGCAGAACAATTTGCTGAGGG |
| | | CTATTGAGGCGCAACAGCATCTGTTGCAACTCACAGTCTGGGGCATCAAG |
| | | CAGCTCCAGGCAAGAATCCTGGCTGTGGAAAGATACCTAAAGGATCAACA |
| | | GCTCCTGGGGATTTGGGGTTGCTCTGGAAAACTCATTTGCACCACTGCTGT |
| | | GCCTTGGAATGCTAGTTGGAGTAATAAATCTCTGGAACAGATTTGGAATCA |
| | | CACGACCTGGATGGAGTGGGACAGAGAAATTAACAATTACACAAGCTTAA |
| | | TACACTCCTTAATTGAAGAATCGCAAAACCAGCAAGAAAAGWRYKCTTCC |
| | | GATCTATTGGAATTAGATAAATGGGCAAGTTTGTGGAATTGGTTTAACATAA |
| | | CAAATTGGCTGTGGTATATAAAATTATTCATAATGATAGTAGGAGGCTTGGT |
| | | AGGTTTAAGAATAGTTTTTGCTGTACTTTCTATAGTGAAT |
| 798 | T cell | CAAAAATTCAAAATTTTCGGGTTTATTACAGGGACAGCAGAGATCCACTTT |
| | activation | GGCGCCGGCTCGAGGGGGCCCGGGTGCAAAGATGGATAAAGTTTTAAAC |
| | (library 1) | AGAGAGGAATCTTTGCAGCTAATGGACCTTCTAGGTCTTGAAAGGAGTGG |
| | backbone | GAATTGGCTCCGGTGCCCGTCAGTGGGCAGAGCGCACATGGCCCACAGTC |
| | | CCCGAGAAGTTGGGGGGAGGGGTCGGCAATTGATCCGGTGCCTAGAGAA |
| | | GGTGGCGCGGGGTAAACTGGGAAAGTGATGTCGTGTACTGGCTCCGCCTT |
| | | TTTCCCGAGGGTGGGGGAGAACCGTATATAAGTGCAGTAGTCGCCGTGAA |
| | | CGTTCTTTTTCGCAACGGGTTTGCCGCCAGAACACAGGTAAGTGCCGTGT |
| | | GTGGTTCCCGCGGGCCTGGCCTCTTTACGGGTTATGGCCCTTGCGTGCCTT |
| | | GAATTACTTCCACCTGGCTGCAGTACGTGATTCTTGATCCCGAGCTTCGGG |
| | | TTGGAAGTGGGTGGGAGAGTTCGAGGCCTTGCGCTTAAGGAGCCCCTTCG |
| | | CCTCGTGCTTGAGTTGAGGCCTGGCCTGGGCGCTGGGGCCGCCGCGTGCG |
| | | AATCTGGTGGCACCTTCGCGCCTGTCTCGCTGCTTTCGATAAGTCTCTAGC |
| | | CATTTAAAATTTTTGATGACCTGCTGCGACGCTTTTTTTCTGGCAAGATAGT |
| | | CTTGTAAATGCGGGCCAAGATCTGCACACTGGTATTTCGGTTTTTGGGGCC |
| | | GCGGGCGGCGACGGGGCCCGTGCGTCCCAGCGCACATGTTCGGCGAGGC |
| | | GGGGCCTGCGAGCGCGGCCACCGAGAATCGGACGGGGGTAGTCTCAAGC |
| | | TGGCCGGCCTGCTCTGGTGCCTGGCCTCGCGCCGCCGTGTATCGCCCCGC |
| | | CCTGGGCGGCAAGGCTGGCCCGGTCGGCACCAGTTGCGTGAGCGGAAAG |
| | | ATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTT |
| | | TCCGTCCTCAGCCGTCGCTTCATGTGACTCCACGGAGTACCGGGCGCCGT |
| | | CCAGGCACCTCGATTAGTTCTCGAGCTTTTGGAGTACGTCGTCTTTAGGTT |
| | | GGGGGGAGGGGTTTTATGCGTTGGAGTTTCCCCACACTGAGTGGGTGGAG |
| | | ACTGAAGTTAGGCCAGCTTGGCACTTGATGTAATTCTCCTTGGAATTTGCC |
| | | CTTTTTGAGTTTGGATCTTGGTTCATTCTCAAGCCTCAGACAGTGGTTCAA |
| | | AGTTTTTTTCTTCCATTTCAGGTGTCGTGACGTACGGCCACCATGACCGAG |
| | | TACAAGCCCACGGTGCGCCTCGCCACCCGCGACGACGTCCCCAGGGCCGT |
| | | ACGCACCCTCGCCGCCGCGTTCGCCGACTACCCCGCCACGCGCCACACCG |
| | | TCGATCCGGACCGCCACATCGAGCGGGTCACCGAGCTGCAAGAACTCTTC |
| | | CTCACGCGCGTCGGGCTCGACATCGGCAAGGTGTGGGTCGCGGACGACG |
| | | GCGCCGCCGTGGCGGTCTGGACCACGCCGGAGAGCGTCGAAGCGGGGGC |
| | | GGTGTTCGCCGAGATCGGCCCGCGCATGGCCGAGTTGAGCGGTTCCCGGC |
| | | TGGCCGCGCAGCAACAGATGGAAGGCCTCCTGGCGCCGCACCGGCCCAA |
| | | GGAGCCCGCGTGGTTCCTGGCCACCGTCGGAGTCTCGCCCGACCACCAGG |
| | | GCAAGGGTCTGGGCAGCGCCGTCGTGCTCCCCGGAGTGGAGGCGGCCGA |
| | | GCGCGCCGGGGTGCCCGCCTTCCTGGAAACCTCCGCGCCCCGCAACCTCC |
| | | CCTTCTACGAGCGGCTCGGCTTCACCGTCACCGCCGACGTCGAGGTGCCC |
| | | GAAGGACCGCGCACCTGGTGCATGACCCGCAAGCCCGGTGCCGAGGGCA |
| | | GGGGAAGTCTGCTAACATGCGGGGACGTGGAGGAAAATCCCGGCCCCGT |
| | | GAGCGAGCTGATTAAGGAGAACATGCACATGAAGCTGTACATGGAGGGCA |
| | | CCGTGAACAACCACCACTTCAAGTGCACATCCGAGGGCGAAGGCAAGCC |
| | | CTACGAGGGCACCCAGACCATGAGAATCAAGGCGGTCGAGGGCGGCCCT |
| | | CTCCCCTTCGCCTTCGACATCCTGGCTACCAGCTTCATGTACGGCAGCAAA |
| | | ACCTTCATCAACCACACCCAGGGCATCCCCGACTTCTTTAAGCAGTCCTTC |
| | | CCCGAGGGCTTCACATGGGAGAGAGTCACCACATACGAGGACGGGGGCG |
| | | TGCTGACCGCTACCCAGGACACCAGCCTCCAGGACGGCTGCCTCATCTAC |
| | | AACGTCAAGATCAGAGGGGTGAACTTCCCATCCAACGGCCCTGTGATGCA |
| | | GAAGAAAACACTCGGCTGGGAGGCCTCCACCGAAACCCTGTACCCCGCT |
| | | GACGGCGGCCTGGAAGGCAGAGCCGACATGGCCCTGAAGCTCGTGGGCG |
| | | GGGGCCACCTGATCTGCAACTTGAAAACCACATACAGATCCAAGAAACCC |
| | | GCTAAGAACCTCAAGATGCCCGGCGTCTACTATGTGGACAGAAGGCTGGA |
| | | AAGAATCAAGGAGGCCGACAAAGAAACCTACGTCGAGCAGCACGAGGTG |
| | | GCTGTGGCCAGATACTGCGACCTCCCTAGCAAACTGGGGCACAGATAAGG |
| | | ATCCACGCGTTAAGTCGACAATCAACCTCTGGATTACAAAATTTGTGAAAG |
| | | ATTGACTGGTATTCTTAACTATGTTGCTCCTTTTACGCTATGTGGATACGCTG |
| | | CTTTAATGCCTTTGTATCATGCTATTGCTTCCCGTATGGCTTTCATTTTCTCC |
| | | TCCTTGTATAAATCCTGGTTGCTGTCTCTTTATGAGGAGTTGTGGCCCGTTG |
| | | TCAGGCAACGTGGCGTGGTGTGCACTGTGTTTGCTGACGCAACCCCCACT |
| | | GGTTGGGGCATTGCCACCACCTGTCAGCTCCTTTCCGGGACTTTCGCTTTC |
| | | CCCCTCCCTATTGCCACGGCGGAACTCAACGCCGCCTGCCTTGCCCGCTG |
| | | CTGGACAGGGGCTCGGCTGTTGGGCACTGACAATTCCGTGGTGTTGTCGG |
| | | GGAAATCATCGTCCTTTCCTTGGCTGCTCGCCTGTGTTGCCACCTGGATTC |
| | | TGCGCGGGACGTCCTTCTGCTACGTCCCTTCGGCCCTCAATCCAGCGGAC |
| | | CTTCCTTCCCGCGGCCTGCTGCCGGCTCTGCGGCCTCTTCCGCGTGTTCGC |
| | | CTTCGCCCTCAGACGAGTCGGATCTCCCTTTGGGCCGCCTCCCCGCGTCG |
| | | ACTTTAAGACCAATGACTTACAAGGCAGCTGTAGATCTTAGCCACTTTTTA |
| | | AAAGAAAAGGGGGGACTGGAAGGGCTAATTCACTCCCAACGATGACAAG |
| | | ATCTGCTTTTTGCTTGTACTGGCTCTCTCTGGTTAGACCAGATCTGAGCCTG |
| | | GGAGCTCTCTGGCTAACTAGGGAACCCACTGCTTAAGCCTCAATAAAGCT |
| | | TGCCTTGAGTGCTTCAAGTAGTGTGTGCCCGTCTGTTGTGTGACTCTGGTA |
| | | ACTAGAGATCCCTCAGACCCTTTTAGTCAGTGTGGAAAATCTCTAGCAGTA |
| | | CGTATAGTAGTTCATGTCATCTTATTATTCAGTATTTATAACTTGCAAAGAAA |
| | | TGAATATCAGAGAGTGAGAGGAACTTGTTTATTGCAGCTTATAATGGTTAC |
| | | AAATAAAGCAATAGCATCACAAATTTCACAAATAAAGCATTTTTTTCACTG |
| | | CATTCTAGTTGTGGTTTGTCCAAACTCATCAATGTATCTTATCATGTCTGGC |
| | | TCTAGCTATCCCGCCCCTAACTCCGCCCATCCCGCCCCTAACTCCGCCCAG |
| | | TTCCGCCCATTCTCCGCCCCATGGCTGACTAATTTTTTTTATTTATGCAGAG |
| | | GCCGAGGCCGCCTCGGCCTCTGAGCTATTCCAGAAGTAGTGAGGAGGCTT |
| | | TTTTGGAGGCCTAGGGACGTACCCAATTCGCCCTATAGTGAGTCGTATTAC |
| | | GCGCGCTCACTGGCCGTCGTTTTACAACGTCGTGACTGGGAAAACCCTGG |
| | | CGTTACCCAACTTAATCGCCTTGCAGCACATCCCCCTTTCGCCAGCTGGCG |
| | | TAATAGCGAAGAGGCCCGCACCGATCGCCCTTCCCAACAGTTGCGCAGCC |
| | | TGAATGGCGAATGGGACGCGCCCTGTAGCGGCGCATTAAGCGCGGCGGGT |
| | | GTGGTGGTTACGCGCAGCGTGACCGCTACACTTGCCAGCGCCCTAGCGCC |
| | | CGCTCCTTTCGCTTTCTTCCCTTCCTTTCTCGCCACGTTCGCCGGCTTTCCC |
| | | CGTCAAGCTCTAAATCGGGGGCTCCCTTTAGGGTTCCGATTTAGTGCTTTA |
| | | CGGCACCTCGACCCCAAAAAACTTGATTAGGGTGATGGTTCACGTAGTGG |
| | | GCGATCGCCCTGATAGACGGTTTTTCGCCCTTTGACGTTGGAGTCCACGTT |
| | | CTTTAATAGTGGACTCTTGTTCCAAACTGGAACAACACTCAACCCTATCTC |
| | | GGTCTATTCTTTTGATTTATAAGGGATTTTGCCGATTTCGGCCTATTGGTTAA |
| | | AAAATGAGCTGATTTAACAAAAATTTAACGCGAATTTTAACAAAATATTAA |
| | | CGCTTACAATTTAGGTGGCACTTTTCGGGGAAATGTGCGCGGAACCCCTAT |
| | | TTGTTTATTTTTCTAAATACATTCAAATATGTATCCGCTCATGAGACAATAAC |
| | | CCTGATAAATGCTTCAATAATATTGAAAAAGGAAGAGTATGAGTATTCAAC |
| | | ATTTCCGTGTCGCCCTTATTCCCTTTTTTGCGGCATTTTGCCTTCCTGTTTTT |
| | | GCTCACCCAGAAACGCTGGTGAAAGTAAAAGATGCTGAAGATCAGTTGG |
| | | GTGCACGAGTGGGTTACATCGAACTGGATCTCAACAGCGGTAAGATCCTT |
| | | GAGAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAACTC |
| | | GGTCGCCGCATACACTATTCTCAGAATGACTTGGTTGAGTACTCACCAGTC |
| | | ACAGAAAAGCATCTTACGGATGGCATGACAGTAAGAGAATTATGCAGTGC |
| | | TGCCATAACCATGAGTGATAACACTGCGGCCAACTTACTTCTGACAACGAT |
| | | CGGAGGACCGAAGGAGCTAACCGCTTTTTTGCACAACATGGGGGATCATG |
| | | TAACTCGCCTTGATCGTTGGGAACCGGAGCTGAATGAAGCCATACCAAAC |
| | | GACGAGCGTGACACCACGATGCCTGTAGCAATGGCAACAACGTTGCGCAA |
| | | ACTATTAACTGGCGAACTACTTACTCTAGCTTCCCGGCAACAATTAATAGA |
| | | CTGGATGGAGGCGGATAAAGTTGCAGGACCACTTCTGCGCTCGGCCCTTC |
| | | CGGCTGGCTGGTTTATTGCTGATAAATCTGGAGCCGGTGAGCGTGGGTCCC |
| | | GCGGTATCATTGCAGCACTGGGGCCAGATGGTAAGCCCTCCCGTATCGTAG |
| | | TTATCTACACGACGGGGAGTCAGGCAACTATGGATGAACGAAATAGACAG |
| | | ATCGCTGAGATAGGTGCCTCACTGATTAAGCATTGGTAACTGTCAGACCAA |
| | | GTTTACTCATATATACTTTAGATTGATTTAAAACTTCATTTTTAATTTAAAAG |
| | | GATCTAGGTGAAGATCCTTTTTGATAATCTCATGACCAAAATCCCTTAACGT |
| | | GAGTTTTCGTTCCACTGAGCGTCAGACCCCGTAGAAAAGATCAAAGGATC |
| | | TTCTTGAGATCCTTTTTTTCTGCGCGTAATCTGCTGCTTGCAAACAAAAAA |
| | | ACCACCGCTACCAGCGGTGGTTTGTTTGCCGGATCAAGAGCTACCAACTC |
| | | TTTTTCCGAAGGTAACTGGCTTCAGCAGAGCGCAGATACCAAATACTGTTC |
| | | TTCTAGTGTAGCCGTAGTTAGGCCACCACTTCAAGAACTCTGTAGCACCGC |
| | | CTACATACCTCGCTCTGCTAATCCTGTTACCAGTGGCTGCTGCCAGTGGCG |
| | | ATAAGTCGTGTCTTACCGGGTTGGACTCAAGACGATAGTTACCGGATAAGG |
| | | CGCAGCGGTCGGGCTGAACGGGGGGTTCGTGCACACAGCCCAGCTTGGA |
| | | GCGAACGACCTACACCGAACTGAGATACCTACAGCGTGAGCTATGAGAAA |
| | | GCGCCACGCTTCCCGAAGGGAGAAAGGCGGACAGGTATCCGGTAAGCGG |
| | | CAGGGTCGGAACAGGAGAGCGCACGAGGGAGCTTCCAGGGGGAAACGC |
| | | CTGGTATCTTTATAGTCCTGTCGGGTTTCGCCACCTCTGACTTGAGCGTCG |
| | | ATTTTTGTGATGCTCGTCAGGGGGGCGGAGCCTATGGAAAAACGCCAGCA |
| | | ACGCGGCCTTTTTACGGTTCCTGGCCTTTTGCTGGCCTTTTGCTCACATGTT |
| | | CTTTCCTGCGTTATCCCCTGATTCTGTGGATAACCGTATTACCGCCTTTGAG |
| | | TGAGCTGATACCGCTCGCCGCAGCCGAACGACCGAGCGCAGCGAGTCAG |
| | | TGAGCGAGGAAGCGGATGAGCGCCCAATACGCAAACCGCCTCTCCCCGC |
| | | GCGTTGGCCGATTCATTAATGCAGCTGGCACGACAGGTTTCCCGACTGGA |
| | | AAGCGGGCAGTGAGCGCAACGCAATTAATGTGAGTTAGCTCACTCATTAG |
| | | GCACCCCAGGCTTTACACTTTATGCTTCCGGCTCGTATGTTGTGTGGAATT |
| | | GTGAGCGGATAACAATTTCACACAGGAAACAGCTATGACCATGATTACGC |
| | | CAAGCGCGCAATTAACCCTCACTAAAGGGAACAAAAGCTGGAGCTGCAA |
| | | GCTTAATGTAGTCTTATGCAATACTCTTGTAGTCTTGCAACATGGTAACGAT |
| | | GAGTTAGCAACATGCCTTACAAGGAGAGAAAAAGCACCGTGCATGCCGAT |
| | | TGGTGGAAGTAAGGTGGTACGATCGTGCCTTATTAGGAAGGCAACAGACG |
| | | GGTCTGACATGGATTGGACGAACCACTGAATTGCCGCATTGCAGAGATATT |
| | | GTATTTAAGTGCCTAGCTCGATACATAAACGGCTCTCTCTGGTTAGACCAG |
| | | ATCTGAGCCTGGGAGCTCTCTGGCTAACTAGGGAACCCACTGCTTAAGCC |
| | | TCAATAAAGCTTGCCTTGAGTGCTTCAAGTAGTGTGTGCCCGTCTGTTGTG |
| | | TGACTCTGGTAACTAGAGATCCCTCAGACCCTTTTAGTCAGTGTGGAAAAT |
| | | CTCTAGCAGTGGCGCCCGAACAGGGACTTGAAAGCGAAAGGGAAACCAG |
| | | AGGAGCTCTCTCGACGCAGGACTCGGCTTGCTGAAGCGCGCACGGCAAG |
| | | AGGCGAGGGGCGGCGACTGGTGAGTACGCCAAAAATTTTGACTAGCGGA |
| | | GGCTAGAAGGAGAGAGATGGGTGCGAGAGCGTCAGTATTAAGCGGGGGA |
| | | GAATTAGATCGCGTTGGGAAAAAATTCGGTTAAGGCCAGGGGGAAAGAA |
| | | AAAATATAAATTAAAACATATAGTATGGGCAAGCAGGGAGCTAGAACGATT |
| | | CGCAGTTAATCCTGGCCTGTTAGAAACATCAGAAGGCTGTAGACAAATACT |
| | | GGGACAGCTACAACCATCCCTTCAGACAGGATCAGAAGAACTTAGATCAT |
| | | TATATAATACAGTAGCAACCCTCTATTGTGTGCATCAAAGGATAGAGATAAA |
| | | AGACACCAAGGAAGCTTTAGACAAGATAGAGCAAGAGCAAAACAAAAGT |
| | | AAGACCACCGCACAGCAAGCGGCCGCTGATCTTCAGACCTGGAGGAGGA |
| | | GATATGAGGGACAATTGGAGAAGTGAATTATATAAATATAAAGTAGTAAAA |
| | | ATTGAACCATTAGGAGTAGCACCCACCAAGGCAAAGAGAAGAGTGGTGC |
| | | AGAGAGAAAAAAGAGCAGTGGGAATAGGAGCTTTGTTCCTTGGGTTCTTG |
| | | GGAGCAGCAGGAAGCACTATGGGCGCAGCGTCAATGACGCTGACGGTAC |
| | | AGGCCAGACAATTATTGTCTGGTATAGTGCAGCAGCAGAACAATTTGCTGA |
| | | GGGCTATTGAGGCGCAACAGCATCTGTTGCAACTCACAGTCTGGGGCATC |
| | | AAGCAGCTCCAGGCAAGAATCCTGGCTGTGGAAAGATACCTAAAGGATCA |
| | | ACAGCTCCTGGGGATTTGGGGTTGCTCTGGAAAACTCATTTGCACCACTG |
| | | CTGTGCCTTGGAATGCTAGTTGGAGTAATAAATCTCTGGAACAGATTTGGA |
| | | ATCACACGACCTGGATGGAGTGGGACAGAGAAATTAACAATTACACAAGC |
| | | TTAATACACTCCTTAATTGAAGAATCGCAAAACCAGCAAGAAAAGAATGA |
| | | ACAAGAATTATTGGAATTAGATAAATGGGCAAGTTTGTGGAATTGGTTTAA |
| | | CATAACAAATTGGCTGTGGTATATAAAATTATTCATAATGATAGTAGGAGGC |
| | | TTGGTAGGTTTAAGAATAGTTTTTGCTGTACTTTCTATAGTGAATAGAGTTA |
| | | GGCAGGGATATTCACCATTATCGTTTCAGACCCACCTCCCAACCCCGAGGG |
| | | GACCCAGAGAGGGCCTATTTCCCATGATTCCTTCATATTTGCATATACGATA |
| | | CAAGGCTGTTAGAGAGATAATTAGAATTAATTTGACTGTAAACACAAAGAT |
| | | ATTAGTACAAAATACGTGACGTAGAAAGTAATAATTTCTTGGGTAGTTTGC |
| | | AGTTTTAAAATTATGTTTTAAAATGGACTATCATATGCTTACCGTAACTTGA |
| | | AAGTATTTCGATTTCTTGGCTTTATATATCTTGTGGAAAGGACGAAACACCG |
| | | GAGACGGTTGTAAAGATATCTGAGCACACAAAATACAGAATTCCATGCTAA |
| | | AATATTGTTCCTGTTCCATTAACTGCGCTAAAAATAAACGTCTCTGTTTGAG |
| | | AGCTATGCTGGAAACAGCATAGCAAGTTCAAATAAGGCTAGTCCGTTATCA |
| | | ACTTGAAAAAGTGGCACCGAGTCGGTGCTCGGAAAAAAAAGAAAAAAA |
| | | GAAAAAAAGAAAAAAATCCATGACCAAATTGGTCCATGGATTCACCCACC |
| | | CCTTTTTATTTTTTTCACCGTGGGCAGGTGAATCGATACACCTGCATGGGC |
| | | GATTCTAGAAAGCTTGGCGTAACTAGATCTTGAGACAAATGGCAGTATTCA |
| | | TCCACAATTTTAAAAGAAAAGGGGGGATTGGGGGGTACAGTGCAGGGGA |
| | | AAGAATAGTAGACATAATAGCAACAGACATACAAACTAAAGAATTACAAA |
| | | AACAAATTA |

In some embodiments, the disclosure relates to a nucleic acid sequence encoding one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247 and one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526.

In some embodiments, the disclosure relates to a nucleic acid molecule comprising (i) a nucleic acid sequence encoding one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247 and one or more anti-CD20 amino acid sequence comprising the amino acid sequence of one to six of SEQ ID NOS: 504-526, or a functional variant thereof at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526 and (ii) a backbone sequence comprising the sequence of SEQ ID NOS: 796, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 796.

In some embodiments, the disclosure relates to a nucleic acid molecule comprising (i) a nucleic acid sequence encoding one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247 and one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526 and (ii) a backbone sequence comprising the sequence of SEQ ID NOS: 797, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 797.

In some embodiments, the disclosure relates to a nucleic acid molecule comprising (i) a nucleic acid sequence encoding one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247 and one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526 and (ii) a backbone sequence comprising the sequence of SEQ ID NOS: 798, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 798.

In some embodiments, the disclosure relates to a nucleic acid sequence encoding: (i) one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247 and a KIR2DS2 transmembrane domain comprising SEQ ID NO: 753, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753 and (ii) one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526 and a KIR2DS2 transmembrane domain comprising SEQ ID NO: 753, or a functional variant thereof comprising at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753.

In some embodiments, the disclosure relates to a nucleic acid sequence encoding: (i) one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743 and (ii) one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743.

In some embodiments, the disclosure relates to a nucleic acid sequence encoding: (i) one or more an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743 and (ii) one or more anti-CD20 amino acid sequence comprising the amino acid sequence of one to six of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743.

In some embodiments, the disclosure relates to a nucleic acid sequence encoding: (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743; (ii) an anti-CD20 amino acid sequence comprising the amino acid sequence of one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743; and (iii) a DAP12 amino acid sequence comprising the amino acid sequence of SEQ ID NO: 750, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 750.

In some embodiments, the disclosure relates to a nucleic acid sequence encoding: (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, an NKG2D transmembrane domain comprising SEQ ID NO: 795, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 795, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794; (ii) an anti-CD20 amino acid sequence comprising the amino acid sequence of one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, an NKG2D transmembrane domain comprising SEQ ID NO: 795, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 795, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743; and (iii) a DAP10 amino acid sequence comprising the amino acid sequence of SEQ ID NO: 789, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 789.

In some embodiments, the disclosure relates to a nucleic acid sequence encoding: (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743; (ii) an anti-CD20 amino acid sequence comprising the amino acid sequence one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and an KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743; and (iii) a DAP12 amino acid sequence comprising the amino acid sequence of SEQ ID NO: 750, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 750.

In some embodiments, the disclosure relates to a nucleic acid sequence encoding: (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794; (ii) an anti-CD20 amino acid sequence comprising the amino acid sequence one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794; and (iii) a DAP10 amino acid sequence comprising the amino acid sequence of SEQ ID NO: 789, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 789.

In some embodiments, the disclosure relates to a nucleic acid sequence encoding: (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743; (ii) an anti-CD20 amino acid sequence comprising the amino acid sequence one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and (iii) a DAP12 amino acid sequence comprising the amino acid sequence of SEQ ID NO: 749, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 749.

In some embodiments, the disclosure relates to a nucleic acid sequence encoding (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, (ii) an anti-CD20 amino acid sequence comprising the amino acid sequence one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, and (iii) a DAP10 amino acid sequence comprising the amino acid sequence of SEQ ID NO: 788, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 788.

In some embodiments, the disclosure relates to a nucleic acid sequence encoding (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, (ii) an anti-CD20 amino acid sequence comprising the amino acid sequence one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743.

In some embodiments, the disclosure relates to a nucleic acid sequence encoding (i) an anti-CD19 amino acid sequence comprising the amino acid sequence of one of SEQ ID NOS: 44-86, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to one of SEQ ID NOS: 44-86, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and (ii) an anti-CD20 amino acid sequence comprising the amino acid sequence one of SEQ ID NOS: 459-503, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 459-503, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and further comprising the sequence of one of SEQ ID NOS: 796, 797, or 798, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 796, 797, or 798.

### Polypeptides

The disclosure relates to a polypeptide comprising any amino acid sequence described herein or a functional variant thereof. In some embodiments, the polypeptide is a fusion protein. In some embodiments the fusion protein is the first fusion protein. In some embodiments, compositions comprise a first and a second fusion protein. In some embodiments, the disclosure relates to a composition comprising a polypeptide herein. In some embodiments, the disclosure relates to a composition comprising a fusion protein herein.

In some embodiments, the disclosure relates to an amino acid sequence comprising a first CAR and second CAR, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743.

In some embodiments, the disclosure relates to an amino acid sequence comprising a first CAR, a second CAR and a DAP12 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and the DAP12 amino acid sequence comprising the amino acid sequence of SEQ ID NO: 749, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 749.

In some embodiments, the disclosure relates to an amino acid sequence comprising a first CAR, a second CAR and a DAP10 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, an NKG2D transmembrane domain comprising SEQ ID NO: 795, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 795, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794; the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, an NKG2D transmembrane domain comprising SEQ ID NO: 795, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 795, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, and the DAP10 amino acid sequence comprising the amino acid sequence of SEQ ID NO: 788, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 788.

In some embodiments, the disclosure relates to an amino acid sequence comprising a first CAR, a second CAR and a DAP12 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and the DAP12 amino acid sequence comprising the amino acid sequence of SEQ ID NO: 749, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 749.

In some embodiments, the disclosure relates to an amino acid sequence comprising a first CAR, a second CAR and a DAP10 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794; the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, and the DAP10 amino acid sequence comprising the amino acid sequence of SEQ ID NO: 788, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 788.

In some embodiments, the disclosure relates to a fusion protein comprising an antigen-binding domain comprising an anti-CD19 amino acid sequence; a transmembrane domain comprising a KIR polypeptide; and a cytoplasmic domain comprising a KIR polypeptide capable of associating with DAP12. In some embodiments, the anti-CD19 amino acid sequence comprises one or more CDR amino acid sequence of one of SEQ ID NOS: 87-247 or a functional variant thereof that have at least about 70%, about 75%, about 80%, about 85%, about 90%, about 91%, about 92%, about 93%, about 94%, about 95%, about 96%, about 97%, about 98%, about 99% or more sequence identity to the respective amino acid sequence(s) of SEQ ID NOS: 87-247. In some embodiments, the anti-CD19 amino acid sequence comprises one or more CDR amino acid sequence of GNS, AND, ANI, SDG, GSS, GNT, GYN, ADS, GSH, KDT, SSS, LVS, QVS, RDT, SVS, NND, SNK, and EAS. In some embodiments, the transmembrane domain and cytoplasmic domain comprise a KIR2DS2 domain. In some embodiments, the transmembrane domain comprises a DAP12 association sequence capable of associating with DAP12 and comprising SEQ ID NO: 753 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753. In some embodiments, the transmembrane domain of the first nucleic acid sequence is configured to associate with DAP12 if the antigen-binding domain is associated with CD19. In some embodiments, at least a portion of the cytoplasmic domain is in an active state when CD19 is bound to the antigen-binding domain.

In some embodiments, the fusion protein further comprises a second antigen binding domain comprising an anti-CD20 amino acid sequence comprising one or more CDR amino acid sequence of one of SEQ ID NOS: 504-526 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective amino acid sequence(s) of SEQ ID NOS: 504-526. In some embodiments, the anti-CD20 amino acid sequence comprises one or more CDR amino acid sequences of SEQ ID NOS: 504-526.

In some embodiments, the second antigen binding domain comprises the anti-CD20, or CD20 targeting, amino acid sequence and the first and second antigen binding domains are non-contiguous and linked by a cleavable linker/ribosome skipping sequence. In some embodiments, the cleavable linker/ribosome skipping sequence is a P2A linker or a furin sequence. In some embodiments, the cleavable linker/ribosome skipping sequence is selected from P2A, E2A, T2A, F2A, IRES. In some embodiments, the non-contiguous first and second nucleic acid sequences are linked by a furin site.

In some embodiments, the fusion protein comprises, in an amino to carboxy orientation: an IgK leader sequence, the first antigen binding domain, the transmembrane domain, the cytoplasmic domain, a furin linker, and the second antigen binding domain. In some embodiments, the fusion protein comprises, in an amino to carboxy orientation: an IgK leader sequence, the first antigen binding domain, the transmembrane domain, the cytoplasmic domain, a furin linker, and the second antigen binding domain, a second transmembrane domain, and a second cytoplasmic domain.

In some embodiments, the anti-CD19 amino acid sequence comprises an scFv domain comprising the amino acid sequence of one of SEQ ID NOS: 271-356 or a functional variant thereof that comprises at least about 75% sequence identity to the respective one of SEQ ID NOS: 271-356.

In some embodiments, the fusion protein comprises, in an amino to carboxy orientation: an IgK leader sequence, the first antigen binding domain, the transmembrane domain, the cytoplasmic domain, a furin or a P2A or T2A linker, a second nucleic acid sequence comprising an anti-CD20 amino acid sequence, a second transmembrane domain that comprises a KIR polypeptide configured to associate with DAP12, and a second cytoplasmic domain that comprises a second KIR polypeptide.

In some embodiments, the fusion protein further comprising a first extracellular linker domain between the first antigen binding domain and the transmembrane domain. In some embodiments, the first extracellular linker comprises the amino acid sequence of SEQ ID NO: 260. In some embodiments, the fusion protein further comprises a second extracellular linker domain between the second antigen binding domain and the second transmembrane domain. In some embodiments, the second extracellular linker comprises the amino acid sequence of SEQ ID NO: 260.

In some embodiments, the fusion protein has a molecular weight of no more than about 22,000 kD.

### Cells

In some embodiments, the disclosure relates to a cell comprising a first exogenous protein and a second exogenous protein. In some embodiments, the first exogenous protein comprises the first fusion protein. In some embodiments, the first exogenous protein is the first fusion protein. In some embodiments, the first exogenous protein comprises a fusion protein herein. In some embodiments, the second exogenous protein comprises the second fusion protein. In some embodiments, the second exogenous protein is the second fusion protein. In some embodiments, the second exogenous protein comprises a fusion protein herein. In some embodiments, the disclosure relates to a plurality of cells comprising two or more of any one type of cell herein. In some embodiments, the disclosure relates to a composition comprising a plurality of cells herein.

In some embodiments, the cell comprises a first and a second exogenous protein. In some embodiments, the first exogenous protein comprises a first antigen-binding domain comprising an anti-CD19 amino acid sequence; a first transmembrane domain comprising a KIR polypeptide configured to associate with DAP12; and a cytoplasmic domain comprising a KIR polypeptide. In some embodiments, the second exogenous protein comprises a second antigen-binding domain comprising an anti-CD20 amino acid sequence; a second transmembrane domain comprising a KIR polypeptide configured to associate with DAP12; and a second cytoplasmic domain comprising a KIR polypeptide.

In some embodiments, the cell further comprises a DAP12 polypeptide.

In some embodiments, the anti-CD19 amino acid sequence comprises one or more CDR amino acid sequence of SEQ ID NOS: 87-247 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective amino acid sequence(s) of SEQ ID NOS: 87-247. In some embodiments, the anti-CD19 amino acid sequence comprises one or more CDR amino acid sequence of one of GNS, AND, ANI, SDG, GSS, GNT, GYN, ADS, GSH, KDT, SSS, LVS, QVS, RDT, SVS, NND, SNK, and EAS. In some embodiments, the anti-CD20 amino acid sequence comprises one or more CDR amino acid sequence of one of SEQ ID NOS: 504-526 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective amino acid sequence(s) of SEQ ID NOS: 504-526. In some embodiments, the anti-CD20 amino acid sequence comprises one or more CDR amino acid sequence of one of SEQ ID NOS: 504-526.

In some embodiments, the anti-CD19 amino acid sequence comprises an scFv domain comprising the amino acid sequence of one of SEQ ID NOS: 271-356 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 271-356. In some embodiments, the anti-CD20 amino acid sequence is a scFv domain comprising the amino acid sequence of one of SEQ ID NOS: 459-503 or a functional variant thereof having at least about 70%, about 75%, about 80%, about 85%, about 90%, about 91%, about 92%, about 93%, about 94%, about 95%, about 96%, about 97%, about 98%, about 99% or more sequence identity to the respective one of SEQ ID NOS: 459-503.

In some embodiments, the first and second transmembrane domains are identical. In some embodiments, the first and second cytoplasmic domains are identical. In some embodiments, the first and second transmembrane domains associate in the presence of CD19 and/or CD20.

In some embodiments, the cell is an immune cell. In some embodiments, the immune cell is a T cell, a B cell, a natural killer (NK) cell, a macrophage, a dendritic cell, a monocyte, or a granulocyte, neutrophil, eosinophil, plasma cell, etc. In some embodiments, the cell is a T cell, a B cell, or an NK cell. In some embodiments, the T cell is a CD8-positive T cell, a CD4-positive T cell, a regulatory T cell (Treg), a cytotoxic T cell (CTL), a central memory T cell (TCM), an effector memory T cell (TEM), a tissue-resident memory T cell (TRM), a stem cell-like memory T cell (TSCM), naïve T cell, natural killer T cell, double negative T cell, or a tumor infiltrating lymphocyte (TIL) In some embodiments, the cell is a tumor infiltrating cell. In some embodiments, the cell is an autologous T cell, B cell, NK cell, monocyte, dendritic cell, or macrophage modified in vitro. In some embodiments, the surface expression of the first and second antigen binding domains are from about 1,000 to about 10,000 copies after about 20 days in vivo. In some embodiments, the surface expression of the first and second antigen binding domains are from about 1,000 to about 100,000 copies after about 20 days in vivo. In some embodiments, the surface expression of the first and second antigen binding domains are from about 100 to about 1,000,000 copies after about 20 days in vivo.

In some embodiments, the disclosure relates to a cell comprising an amino acid sequence comprising a first CAR and second CAR, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743.

In some embodiments, the disclosure relates to a cell comprising an amino acid sequence comprising a first CAR, a second CAR and a DAP12 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 753, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 753, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and the DAP12 amino acid sequence comprising the amino acid sequence of SEQ ID NO: 749, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 749.

In some embodiments, the disclosure relates to a cell comprising an amino acid sequence comprising a first CAR, a second CAR and a DAP10 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, an NKG2D transmembrane domain comprising SEQ ID NO: 795, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 795, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, an NKG2D transmembrane domain comprising SEQ ID NO: 795, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 795, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, and the DAP10 amino acid sequence comprising the amino acid sequence of SEQ ID NO: 788, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 788.

In some embodiments, the disclosure relates to a cell comprising an amino acid sequence comprising a first CAR, a second CAR and a DAP12 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, a KIR2DS2 transmembrane domain comprising SEQ ID NO: 742, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 742, and a KIR2DS2 intracellular domain comprising the amino acid sequence of SEQ ID NO: 743, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 743, and the DAP12 amino acid sequence comprising the amino acid sequence of SEQ ID NO: 749, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 749.

In some embodiments, the disclosure relates to a cell comprising an amino acid sequence comprising a first CAR, a second CAR and a DAP10 amino acid sequence, the first CAR comprising one or more an anti-CD19 amino acid sequence comprising the amino acid sequence of one to six of SEQ ID NOS: 87-247 and GNS, AND, ANI, SDG, GSS, GNT, GYN, ADS, GSH, KDT, SSS, LVS, QVS, RDT, SVS, NND, SNK, and EAS, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 87-247, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, the second CAR comprising one or more anti-CD20 amino acid sequence comprising the amino acid sequence of one to six of SEQ ID NOS: 504-526, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NOS: 504-526, an NKG2D transmembrane domain comprising SEQ ID NO: 793, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 793, and an NKG2D cytoplasmic domain comprising the amino acid sequence of SEQ ID NO: 794, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 794, and the DAP10 amino acid sequence comprising the amino acid sequence of SEQ ID NO: 788, or a functional variant thereof having at least about 70%, about 75%, about 80%, about 85%, about 90%, about 91%, about 92%, about 93%, about 94%, about 95%, about 96%, about 97%, about 98%, about 99% or more sequence identity to SEQ ID NO: 788.

Methods for expanding cells of the disclosure and for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (201 1) Nat Rev Clin Oncol. 8(10):577-85). See, e.g., Themeli et al. (2013) Nat Biotechnol. 31 (10): 928-933; Tsukahara et al. (2013) Biochem Biophys Res Commun 438(1): 84-9; Davila et al. (2013) PLoS ONE 8(4): e61338. Cell therapy may be carried out by autologous transfer, in which the cells are isolated and/or otherwise prepared from the subject who is to receive the cell therapy, or from a sample derived from such a subject. Thus, in one aspect, the cells are derived from a subject, e.g., patient, in need of a treatment and the cells, following isolation and processing are administered to the same subject.

Alternatively, the cell therapy may be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. In alternative embodiments the allogeneic T cells may lack a functional T cell receptor (TCR) and/or human leukocyte antigen (HLA) such as HLA class I and/or HLA class II.

The pharmaceutical composition in some embodiments comprises the cells in amounts effective to treat or prevent the disease or condition, such as a therapeutically effective or prophylactic ally effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The present disclosure also provides a method for providing a cell population which targets CD-19 and/or CD20 comprising the steps of:
i) isolating a cell from a sample isolated from a subject; and
ii) expanding the cell to provide a cell population which targets CD-19 and/or CD20.

In some aspects, the cell is a T cell. In some aspects, the cell is a NK or NKT cell. The sample may be a tumor, blood or tissue sample from the subject. In one embodiment, the sample from which the cell is isolated may be a blood sample, a tumor sample, a tumor-associated lymph node sample or a sample from a metastatic site. When the cell is a T cell, the population of T cells may comprise CD8+ T cells, CD4+ T cells or CD8+ and CD4+ T cells.

The resulting cell population may have an increased number of cells which target CD-19 and/or CD20 (for example, compared with the sample isolated from the subject). The T cell may have been rendered resistant to immune suppression, for example by gene editing of immune regulatory receptors (including but not restricted to Lag 3, PD1, TIGIT, PD-L1, BTLA, CTLA4, TGFbeta Receptor, IL10 Receptor).

In one aspect, a cell may be used which is an engineered cell, such as an engineered immune cell including: a genetically engineered antigen receptor that specifically binds to a target antigen. Cells suitable for modification may be obtained from a subject, such as a mammalian subject including but not limited to a human subject. Cells may be obtained from a number of sources, known to the skilled person, including peripheral blood mononuclear cells, bone marrow, lymph node tissue, cord blood, thymus tissue, tissue from a site of infection, ascites, pleural effusion, spleen tissue and tumors. Suitable cells include cells of the immune system, such as cells of the innate or adaptive immune systems (e.g. myeloid or lymphoid cells, including lymphocytes, such as T cells, NK cells or an NKT cells. Other suitable cells include stem cells, such as multipotent and pluripotent stem cells, including induced pluripotent stem cells (iPSCs). In one aspect the cells are primary cells, such as those isolated directly from a subject and/or isolated from a subject and frozen.

In certain aspects suitable cells may be obtained from a unit of blood collected from a mammalian subject using a number of techniques known to the skilled person such as Ficoll separation. In one aspect cells from the circulating blood of an individual are obtained by apheresis. The apheresis product typically contains lymphocytes, including T cells, monocytes, granulocytes, B cells, other nucleated white blood cells, red blood cell, and platelets. The cells collected by apheresis may be washed to remove the plasma fraction and, optionally, may be placed in an appropriate buffer or medium for subsequent processing steps. Cells may be washed, for example in phosphate buffered saline. As the absence of calcium can lead to increased activation of T cells, in certain embodiments the wash solution lacks calcium and/or magnesium or may lack many or all divalent cations. The cells may be depleted of T regulatory cells (CD25+ cells) using methods known in the art such as the Miltenyi CD25 microbeads. The depletion of CD25+ cells may be performed prior to apheresis or during production of the CAR-expressing cells (for example T cells or NK cells). Other cell types that negatively affect expansion and/or function of cells expressing a CAR may also be removed. Such immune suppressor cells include cells expressing CD14, CD1 1 b, CD33, CD15, or other markers of immune suppressor cells and may be removed from a population of cells by using methods known to the skilled person, such as magnetic immunoadherence or flow cytometry using monoclonal antibodies directed to cell surface markers. The cells may also be depleted of cells which express one or more check-point inhibitors, for example PD1, CD223 (LAG3), PD-L1 and CTLA4, PD-L2, TIM3, CEACAM-1, CEACAM-3, CEACAM-5, VISTA, BTLA, TIGIT, LAIR1, CD160, 2B4, CD80, CD86, CD276 (B7-H3), B7-H4 (VTCN1), CD270 (HVEM or TNFRSF14), KIR, A2aR, MHC class IMHC class II, GALS, adenosine, TGFβ receptor. A cell population may be depleted of cells expressing one or more check-point inhibitors at the same time as depletion of T regulatory cells. Depletion of T regulatory cells, immune suppressor cells and cells expressing check-point inhibitors may be performed at the same time for example using beads expressing multiple antibodies that recognized such T regulatory cells, immune suppressor cells or check-point inhibitors or a mixture of beads expressing such antibodies. For example, an anti-CD25 antibody or fragment thereof, or other binding moiety, and an anti-check-point inhibitor antibody or fragment thereof, or other binding moiety can be attached to the same bead or to a mixture of separate beads for simultaneous depletion. Alternatively, depletion of certain cell types may be performed sequentially. In some aspects, the cells may be positively selected, for example by incubation with anti-CD3/anti-CD28-conjugated beads (e.g. Dynabeads® Human T-Activator CD3/CD28, or other suitable reagents. In some aspects the affinity-based selection is via magnetic-activated cell sorting (MACS) (Miltenyi Biotec, Auburn, CA). MACS systems are capable of high-purity selection of cells having magnetic particles attached to them. MACS can operate in a mode where the target and non-target cells are sequentially eluted after the application of the external magnetic field. Cells attached to the magnetic particles are retained while the unattached cells are eluted then the cells that were held in the magnetic field may also be eluted. In certain aspects a closed system is used to carry out one or more of the isolation, cell preparation, separation, processing, incubation, culture, and/or formulation, for example to reduce handling. Closed systems are described in WO 2009/070003 or WO 2009/072003. Immune cells may be activated and expanded ex vivo or in vitro, by methods known in the art, for example as described in WO 01/62895, WO 2016/196388, EP1586655, U.S. Pat. Nos. 5,199,942, 6,352,694, 7,144,575, 7,067,318, 7,172,869, 7,572,631.

Following collection of mammalian CD34+ hematopoietic stem and progenitor cells from a peripheral blood harvest or from bone marrow explants, such cells may be expanded ex vivo in the presence of certain growth factors. In addition to the growth factors described in WO 92/21402, other factors such as flt3-L, IL-1, IL-3 and c-kit ligand can be used for culturing and expanding cells.

A population of immune cells may be expanded by contact with an agent and/or ligand attached to a surface, for example a CD-19 and/or CD20 antigen, such as presented by an antigen presenting cell. Alternatively, such agent may stimulate the CD3/TCR complex associated signal and the ligand may stimulate a co-stimulatory molecule present on the surface of the cells. In one aspect T cell populations may be stimulated by contact with an anti-CD3 antibody or antigen-binding fragment thereof, or an anti-CD2 antibody immobilized on a surface, or by contact with a protein kinase C activator (e.g. bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody under conditions appropriate for stimulating proliferation of the T cells. To stimulate proliferation of either CD4+ T cells or CD8+ T cells, an anti-CD3 antibody and an anti-CD28 antibody and may be used.

In certain aspects immune cells (such as PBMCs or T cells) are expanded and stimulated by contacting the cells to one of both of an anti-CD3 antibody and IL-2. Cells may be expanded without anti-CD3 or anti-CD28 beads. In some aspects, the two agents are immobilized on beads, either on the same bead or on separate beads. By way of example, the agent providing the primary activation signal is an anti-CD3 antibody or antigen-binding fragment thereof, and the agent providing the co-stimulatory signal is an anti-CD28 antibody or antigen-binding fragment thereof, both agents are co-immobilized to the same bead. In one aspect, equivalent molecular amounts of each antibody are bound to the beads to stimulate CD4+ T cell expansion and T cell growth. In certain aspects, a ratio of CD3:CD28 antibodies is bound to the beads such that an increase in T cell expansion is observed as compared to the expansion observed using such antibodies at a ratio of 1:1. In one aspect the ratio of CD3:CD28 antibody bound to the beads ranges from between 100:1 to 1:100. In some aspects, more anti-CD28 antibody than anti-CD3 antibody is used. In some aspects, the isolated T cells are not stimulated by CD28 antibody or antibody fragments in order to expand prior to administration.

### Pharmaceutical Compositions

In some embodiments, the disclosure relates to a pharmaceutical compositions comprising one or more of any composition, nucleic acid, polypeptide, or cell herein and a pharmaceutically acceptable carrier. In some embodiments, the pharmaceutically acceptable carrier comprises one or more of an excipient, a carrier, a diluent, and a stabilizer.

In some embodiments, a pharmaceutical composition herein comprises a therapeutically effective amount of a cell herein and a pharmaceutically acceptable carrier. In some embodiments, the therapeutically effective amount is from about 1×10⁶ to about 100×10⁶ cells. In some embodiments, the therapeutically effective amount is from about 10×10⁶ to about 50×10⁶ cells. In some embodiments, the pharmaceutically acceptable carrier comprises phosphate-buffered saline at a pH of about from about 6.5 to about 8.0. In some embodiments, the cells are fresh formulations of cells engineered to comprise one or more nucleic acid sequence, one or more nucleic acid molecule, and/or one or more fusion protein herein. In some embodiments, the cells are cryopreserved for later use and comprise one or more nucleic acid sequence, one or more nucleic acid molecule, and/or one or more fusion protein herein.

In some embodiments, the pharmaceutical compositions comprising one or more of the composition, nucleic acid, polypeptide, or cell herein are in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in some embodiments formulated for intravenous administration. In some embodiments, compositions of the present disclosure can be formulated for intracranial administration and/or spinal injection.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the pharmaceutical composition is substantially free of, e.g., there are no detectable levels of a contaminant, e.g., selected from the group consisting of endotoxin, Mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “or “therapeutic amount” is indicated, the precise amount of the compositions of the present embodiments to be administered can be determined by a physician with consideration of individual differences in age, weight, tumor size, extent of infection or metastasis, and condition of the patient (subject).

### Methods

In some embodiments, the disclosure relates to a method of treatment of a B cell-mediated disorder in a subject in need thereof. In some embodiments, the method comprises administering to the subject a pharmaceutical composition herein. In some embodiments, the method is free of CD28 stimulation. In some embodiments, the method further comprises a step administering a chemotherapeutic agent simultaneously with, before, or after administration of the pharmaceutical composition. In some embodiments, the B cell-mediated disorder is a hyperproliferative disorder. In some embodiments, the B cell-mediated disorder is a B cell lymphoma. In some embodiments, the method further comprises a step of allowing a sufficient period of time for the therapeutically effective amount of cells to bind and to kill a target cancer cell. In some embodiments, the method is free of off-target binding from about 3% to about 10% of the total association between the therapeutically effective amount of cells and a subject's cells.

In some embodiments, the pharmaceutical composition is administered by intrapleural administration, intravenous administration, intraarterial administration, locoregional administration at the site of a surgical resection or combinations thereof. In some embodiments, the subject is a mammal.

In some embodiments, the subject is a human.

In some embodiments, the therapeutically effective amount of cells is from about 1×10⁶ to about 100×10⁶ cells. In some embodiments, the therapeutically effective amount of cells is from about 1×10⁶ to about 3×10⁸ cells. In some embodiments, the therapeutically effective amount is from about 1×10⁶ to about 3×10¹¹ cells. In some embodiments, the therapeutically effective amount is from about 10×10⁶ to about 50×10⁶ cells.

In some embodiments, the disclosure relates to a method of treating a hyperproliferative disorder in a subject need thereof comprising administering to the subject a pharmaceutical composition disclosed herein. In some embodiments, the disclosure relates to a method of treating B cell lymphoma in a subject need thereof. In some embodiments, the method comprises administering to the subject a pharmaceutical composition herein. In some embodiments, the method is free of CD28 stimulation. In some embodiments, the method further comprises a step administering a chemotherapeutic agent simultaneously with, before or after administration of the pharmaceutical composition.

In some embodiments, the disclosure relates to a method of inducing antigen-specific T cell activation against a cell expressing CD19 and CD20. In some embodiments, the method comprises exposing a cell herein to a cell expressing CD19 and CD20 or an epitope of CD19 and CD20. In some embodiments, the step of exposing is performed in vivo. In some embodiments, the method further comprises transducing a cell with a composition comprising herein comprising a nucleic acid before the step of exposing. In some embodiments, the method further comprises extracting a sample of lymphocytes from a subject and isolating T cells from the sample prior to the step of exposing.

In some embodiments, the disclosure relates to a method of reducing relapse rate of cancer in a subject in need thereof. In some embodiments, the method comprises administering to the subject a pharmaceutical composition herein. In some embodiments, the method is free of CD28 stimulation in the subject. In some embodiments, the method is free of CD28 stimulation in the subject by administration of a CAR comprising an anti-CD28 domain. In some embodiments, the method further comprises a step administering a chemotherapeutic agent simultaneously with, before or after administration of the pharmaceutical composition. In some embodiments, the pharmaceutical composition is administered by intrapleural administration, intravenous administration, intraarterial administration, locoregional administration at the site of a surgical resection or combinations thereof. In some embodiments, the subject is a mammal. In some embodiments, the subject is a human. In some embodiments, the therapeutically effective amount of cells is from about 1×10⁶ to about 100×10⁶ cells. In some embodiments, the therapeutically effective amount is from about 10×10⁶ to about 50×10⁶ cells. In some embodiments, the cancer is B cell lymphoma.

In some embodiments, the disclosure relates to methods for producing an in vitro transcribed RNA encoding a fusion protein(s) herein. The present disclosure also includes a fusion protein(s) encoding RNA construct that can be directly transfected into a cell, where the fusion protein(s) is a fusion protein(s) herein. In some embodiments, the method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In some embodiments, the template includes sequences for the fusion protein(s).

In some embodiments, the fusion protein(s) is or are encoded by a messenger RNA (mRNA). In some embodiments, the mRNA encoding the fusion protein(s) is introduced into an immune effector cell, e.g., a T cell or a NK cell, for production of a KIR CAR-expressing cell, e.g., a KIR CART cell or a KIR CAR NK cell.

In some embodiments, the in vitro transcribed RNA of a fusion protein(s) can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

In some embodiments, the temple for in vitro transcription is fusion protein(s) encoding nucleic acid of the present disclosure. In some embodiments, the nucleic acid is a DNA.

In some embodiments, the DNA to be used for PCR contains an open reading frame. The DNA can be from a naturally occurring DNA sequence from the genome of an organism. In some embodiments, the nucleic acid can include some or all of the 5′ and/or 3′ untranslated regions (UTRs). The nucleic acid can include exons and introns. In some embodiments, the DNA to be used for PCR is a human nucleic acid sequence. In some embodiments, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein(s). The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementarity to regions of the DNA to be used as a template for the PCR. “Substantially complementarity,” as used herein, refers to sequences of nucleotides where a majority or all of the bases in the primer sequence are complementarity, or one or more bases are non-complementarity, or mismatched. Substantially complementarity sequences are able to anneal or hybridize with the intended DNA target under annealing conditions used for PCR. The primers can be designed to be substantially complementarity to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In some embodiments, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementarity to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementarity to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In some embodiments, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA For example, it is known that AU-rich elements in 3′ UTR sequences can decrease the stability of mRNA Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts but does not appear to be required for all RNAs to enable efficient translation. The requirement for Kozak sequences for many mRNAs is known in the art. In some embodiments, the 5′ UTR can be 5′UTR of an RNA virus whose RNA genome is stable in cells. In some embodiments, various nucleotide analogues can be used in the 3′ or 5′ UTR to impede exonuclease degradation of the mRNA.

To enable synthesis of RNA from a DNA template without the need for gene cloning, a promoter of transcription could be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters.

Consensus nucleic acid sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed. RNA In some embodiments, the poly(A) tail is between 100 and 5000 adenosines.

Poly(A) tails of RNAs can be further extended following in vitro transcription with the use of a poly(A) polymerase, such as E. coli polyA polymerase (E-PAP). In some embodiments, increasing the length of a poly(A) tail from 100 nucleotides to between 300 and 400 nucleotides results in about a two-fold increase in the translation efficiency of the RNA Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA 5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-11 (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

In some embodiments, a cell herein is made via viral delivery of a nucleic acid herein.

In some embodiments, non-viral methods can be used to deliver a nucleic acid herein into a cell or tissue or a subject. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1 (201 1): R14-20; Singh et al. Cancer Res. 15 (2008): 2961-2971; Huang et al. Mol. Ther. 16 (2008): 580-589; Grabundzija et al. Mol. Ther. 18 (2010): 1200-1209; Kebriaei et al. Blood. 122.21 (2013): 166; Williams. Molecular Therapy 16.9 (2008): 1515-16; Bell et al. Nat. Protoc. 2.12 (2007): 3153-65; and Ding et al. Cell. 122.3 (2005): 473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al.

Exemplary transposons include a pT2-based transposon. See, e.g., Grabundzija et al. Nucleic Acids Res. 41.3 (2013): 1829-47; and Singh et al. Cancer Res. 68.8 (2008): 2961-2971, all of which are incorporated herein by reference. Exemplary transposases include a Tc1/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a fusion protein(s) or KIR CAR described herein. Provided herein are methods of generating a cell, e.g., T cell or NK cell, that stably expresses a fusion protein(s) and/or KIR CAR described herein, e.g., using a transposon system such as SBTS. In accordance with methods described herein, in some embodiments, one or more nucleic acids, e.g., plasmids, containing the SBTS components are delivered to a cell (e.g., Tor NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a fusion protein(s) or KIR CAR described herein) as well as a nucleic acid sequence encoding a transposase enzyme. In some embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., Tor NK cells, are generated that express a fusion protein(s) or KIR CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases). In some embodiments, use of a non-viral method of delivery permits reprogramming of cells, e.g., Tor NK cells, and direct infusion of the cells into a subject. Advantages of non-viral vectors include but are not limited to the ease and relatively low cost of producing sufficient amounts required to meet a patient population, stability during storage, and lack of immunogenicity.

In some embodiments, a vector herein is derived from a retrovirus; for example a lentivirus. Such a vector may be a suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In some embodiments, the vector comprising a nucleic acid herein is an adenoviral vector (A5/35). In some embodiments, the expression of nucleic acids encoding fusion protein(s) or KIR CARs can be accomplished using of transposons such as sleeping beauty, crisper, CAS9, and zinc finger nucleases. See below June et al. 2009 Nature Reviews Immunology 9. 1 0: 704-716, which is incorporated herein by reference.

In brief summary, the expression of natural or synthetic nucleic acids encoding fusion protein(s) or KIR CARs can be achieved by operably linking a nucleic acid encoding the CAR polypeptide or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of some embodiments herein may also be used for nucleic acid therapy using standard gene delivery protocols. Methods for gene delivery are known in the art. See, e.g., U.S. Pat. Nos. 5,399,346, 5,580,859, 5,589,466, incorporated by reference herein in their entireties. In some embodiments, the disclosure provides a gene therapy vector.

A nucleic acid herein can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1a, ubiquitin C, or phosphoglycerokinase (PGK) promoters.

An example of a promoter that is capable of expressing a fusion protein(s) or KIR CAR transgene in a mammalian T cell is the EF-1 alpha (EF1a) promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009). In some embodiments, the EF1a promoter comprises the sequence as known in the art.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-lex promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a fusion protein(s) or KIR CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10).

However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present disclosure further provides a vector comprising a fusion protein(s) or KIR CAR encoding nucleic acid molecule. In some embodiments, a fusion protein(s) or KIR CAR vector can be directly transduced into a cell, e.g., a T cell or NK cell. In some embodiments, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In some embodiments, the vector is capable of expressing the CAR construct in mammalian T cells or NK cells. In some embodiments, the mammalian T cell is a human T cell.

### Combination Therapies

A cell herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A cell herein and the at least one additional therapeutic agent can be administered simultaneously, in the same or in separate compositions, or sequentially.

For sequential administration, the cell herein can be administered first, and the additional agent can be administered second, or the order of administration can be reversed.

The cell therapy and/or other therapeutic agents, procedures or modalities can be administered during periods of active disorder, or during a period of remission or less active disease. The cell therapy can be administered before the other treatment, concurrently with the treatment, post-treatment, or during remission of the disorder.

When administered in combination, the cell therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the cell therapy, the additional agent (e.g., second or third agent), or all, is lower (e.g., at least 20%, at least 30%, at least 40%, or at least 50%) than the amount or dosage of each agent used individually, e.g., as a monotherapy. In some embodiments, the amount or dosage of the cell therapy, the additional agent (e.g., second or third agent), or all, that results in a desired effect (e.g., treatment of viral disorder) lower (e.g., at least 20%, at least 30%, at least 40%, or at least 50% lower) than the amount or dosage of each agent used individually, e.g., as a monotherapy, required to achieve the same therapeutic effect.

In some embodiments, a cell herein may be used in a treatment regimen in combination with surgery, cytokines, radiation, or chemotherapy such as cytoxan, fludarabine, histone deacetylase inhibitors, demethylating agents, or peptide vaccine, such as that described in Izumoto et al. 2008 J Neurosurg 108:963-971. In some embodiments, a cell herein may be used in combination with a CD20xCD3 bispecific antibody.

In some embodiments, cells herein are combined with other therapeutic agents, such as other anti-viral agents, anti-allergic agents, anti-nausea agents (or anti-emetics), pain relievers, cytoprotective agents, and combinations thereof.

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-viral agents for combination therapies herein include: Non-nucleoside reverse transcriptase inhibitors (NNRTIs) (e.g., efavirenz, rilpivirine (Edurant) and doravirine (Pifeltro)), nucleoside or nucleotide reverse transcriptase inhibitors (NRTIs) (e.g., abacavir (Ziagen), tenofovir disoproxil fumarate (Viread), emtricitabine (Emtriva), lamivudine (Epivir) and zidovudine (Retrovir)), mixes of medicines (e.g., emtricitabine-tenofovir disoproxil fumarate (Truvada) and emtricitabine-tenofovir alafenamide fumarate (Descovy)), protease inhibitors (PIs) (e.g., atazanavir (Reyataz), darunavir (Prezista) and lopinavir-ritonavir (Kaletra)), integrase inhibitors (e.g., bictegravir sodium-emtricitabine-tenofovir alafenamide fumarate (Biktarvy), raltegravir (Isentress), dolutegravir (Tivicay) and cabotegravir (Vocabria)), entry or fusion inhibitors enfuvirtide (Fuzeon) and maraviroc (Selzentry), and newer medicines (e.g., ibalizumab-uiyk (Trogarzo) and fostemsavir (Rukobia).

In some embodiments, a cell herein is administered to a subject in combination with a molecule targeting GITR and/or modulating GITR functions, such as a GITR agonist and/or a GITR antibody that depletes regulatory T cells (Tregs). In some embodiments, the GITR binding molecules and/or molecules modulating GITR functions (e.g., GITR agonist and/or Treg depleting GITR antibodies) are administered prior to the CAR-expressing cell. For example, in some embodiments, the GITR agonist can be administered prior to apheresis of the cells. In some embodiments, the subject has CLL. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In some embodiments, a cell herein is administered to a subject in combination with an mTOR inhibitor, e.g., an mTOR inhibitor described herein, e.g., a rapalog such as everolimus. In some embodiments, the mTOR inhibitor is administered prior to the cell. For example, in some embodiments, the mTOR inhibitor can be administered prior to apheresis of the cells.

In some embodiments, a cell herein is administered to a subject in combination with a GITR agonist, e.g., a GITR agonist described herein. In some embodiments, the GITR agonist is administered prior to the CAR-expressing cell. For example, in some embodiments, the GITR agonist can be administered prior to apheresis of the cells.

In some embodiments, a cell herein is administered to a subject in combination with a protein tyrosine phosphatase inhibitor, e.g., a protein tyrosine phosphatase inhibitor described herein. In some embodiments, the protein tyrosine phosphatase inhibitor is an SUP-1 inhibitor, e.g., an SHP-1 inhibitor described herein, such as, e.g., sodium stibogluconate. In some embodiments, the protein tyrosine phosphatase inhibitor is an SHP-2 inhibitor.

In some embodiments, a cell herein can be used in combination with a kinase inhibitor. In some embodiments this approach can be used to optimize the performance of cells herein in the subject. While not wishing to be bound by theory, it is believed that, in an embodiment, the performance of endogenous, non-modified immune effector cells, e.g., T cells or NK cells, is improved. While not wishing to be bound by theory, it is believed that, in an embodiment, the performance of a fusion protein(s) or KIR CAR expressing cell is improved. In some embodiments, cells, e.g., T cells or NK cells, which have, or will be engineered to express a fusion protein(s) or KIR CAR, can be treated ex vivo by contact with an amount of an mTOR inhibitor that increases the number of PD1 negative immune effector cells, e.g., T cells/NK cells or increases the ratio of PD1 negative immune effector cells, e.g., T cells/NK cells/PD1 positive immune effector cells, e.g., T cells or NK cells.

In some embodiments, administration of a low, immune enhancing, dose of an mTOR inhibitor, e.g., an allosteric inhibitor, e.g., RAD00I, or a catalytic inhibitor, is initiated prior to administration of a cell herein, e.g., T cells or NK cells. In some embodiments, the cells are administered after a sufficient time, or sufficient dosing, of an mTOR inhibitor, such that the level of PD1 negative immune effector cells, e.g., T cells/NK cells, or the ratio of PD1 negative immune effector cells, e.g., T cells/NK cells/PD1 positive immune effector cells, e.g., T cells or NK cells, has been, at least transiently, increased.

In some embodiments, the cell, e.g., T cell or NK cell, to be engineered to express a fusion protein(s) or KIR CAR, is harvested after a sufficient time, or after sufficient dosing of the low, immune enhancing, dose of an mTOR inhibitor, such that the level of PD1 negative immune effector cells, e.g., T cells or NK cells, or the ratio of PD1 negative immune effector cells, e.g., T cells/NK cells/PD 1 positive immune effector cells, e.g., T cells or NK cells, in the subject or harvested from the subject has been, at least transiently, increased.

In some embodiments, the mTOR inhibitor is administered for an amount of time sufficient to decrease the proportion of PD-1 positive T cells, increase the proportion of PD-1 negative T cells, or increase the ratio of PD-1 negative T cells/PD-1 positive T cells, in the peripheral blood of the subject, or in a preparation of T cells isolated from the subject.

In some embodiments, the dose of an mTOR inhibitor is associated with mTOR inhibitor of at least 5 but no more than 90%, e.g., as measured by p70 S6K inhibition. In some embodiments, the dose of an mTOR inhibitor is associated with mTOR inhibition of at least 10% but no more than 40%, e.g., as measured by p70 S6K inhibition.

In some embodiments, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In some embodiments, the kinase inhibitor is a BTK inhibitor, e.g., a BTK inhibitor described herein, such as, e.g., ibrutinib or acalabrutinib. In some embodiments, the kinase inhibitor is an mTOR inhibitor, e.g., an mTOR inhibitor described herein, such as, e.g., rapamycin, a rapamycin analog, OSI-027. The mTOR inhibitor can be, e.g., a mTORC1 inhibitor and/or an mTORC2 inhibitor, e.g., a mTORC1 inhibitor and/or mTORC2 inhibitor described herein. In some embodiments, the kinase inhibitor is a MNK inhibitor, e.g., a MNK inhibitor described herein, such as, e.g., 4-amino-5-(4-fluoroanilino)-pyrazolo [3,4-d]pyrimidine. The MNK inhibitor can be, e.g., a MNK1a, MNK1b, MNK2a and/or MNK2b inhibitor. In some embodiments, the kinase inhibitor is a DGK inhibitor, e.g., a DGK inhibitor described herein, such as, e.g., DGKinh1 (D5919) or DGKinh2 (D5794). In some embodiments, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT75 1 9); 4-[2-methyl-1-(1-methylethyl)-1H-imidazol-5-yl]-N-[4-(methylsulfonyl)phenyl]-2-pyrimidinamine (AZD5438); and XL281 (BMS908662).

In some embodiments, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In some embodiments, 1, 2, 3, 4, 5, 6, 7, 8, 9, 10, 11, 12 or more cycles of palbociclib are administered.

In some embodiments, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13.

In some embodiments, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered.

In some embodiments, the kinase inhibitor is an mTOR inhibitor selected from temsirolimus; ridaforolimus (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.049 hexatriaconta-16,24,26,28-tetraen-12-yl]propyl]-2-methoxycyclohexyl dimethylphosphinate, also known as AP23573 and MK.8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-mmino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-, inner salt (SF1 126); and XL765.

In some embodiments, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle, or daily for 28 day cycle. In some embodiments, 1, 2, 3, 4, 5, 6, 7, 8, 9, 10, 11, 12 or more cycles of rapamycin are administered. In some embodiments, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In some embodiments, 1, 2, 3, 4, 5, 6, 7, 8, 9, 10, 11, 12 or more cycles of everolimus are administered.

In some embodiments, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further embodiment, the cell compositions of the present disclosure may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In some embodiments, the cell compositions of the present disclosure are administered following B-cell ablative therapy such as agents that react with CD20, e.g., Rituxan. For example, In some embodiments, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

Some patients may experience allergic reactions to the compounds of the present disclosure and/or other anti-viral agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present disclosure and/or other anti-viral agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof. In some embodiments, methods of the disclosure are free of a step of treating the subject with steroids before or after the step of administration of the pharmaceutical composition.

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct 20 therapy. In some embodiments, cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present disclosure, can be prepared and administered as described in the art, such as in the documents cited above.

In some embodiments, the disclosure relates to a kit. In some embodiments, the kit comprises one or more nucleic acid, fusion protein, nucleic acid molecule, polypeptide, or cell herein and a combination partner as disclosed herein are provided. Representative kits include (a) one or more nucleic acid, fusion protein, nucleic acid molecule, polypeptide, or cell herein or a pharmaceutically acceptable salt thereof, (b) at least one combination partner, e.g., as indicated above, whereby such kit may comprise a package insert or other labeling including directions for administration.

In some embodiments, the subject can be administered an agent which reduces or ameliorates a side effect associated with the administration of a fusion protein(s) or KIR CAR-expressing cell. Side effects may include, but are not limited to CRS, and hemophagocytic lymphohistiocytosis (HLH), also termed Macrophage Activation Syndrome (MAS). Symptoms of CRS include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardiac output (early) and potentially diminished cardiac output (late). CRS may include clinical coagulation signs and symptoms such as elevated cl-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures. Accordingly, the methods described herein can comprise administering a fusion protein(s) or KIR CAR-expressing cell herein, and/or nucleic acid sequences disclosed herein to a subject, and further administering one or more agents to manage elevated levels of a soluble factor resulting from the treatment with the cell. In some embodiments, the soluble factor elevated in the subject is one or more of IFN-γ, TNFα, IL-2 and IL-6. In some embodiments, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In some embodiments, the agent that neutralizes one or more of these soluble forms is an antibody or antigen binding fragment thereof. Examples of such agents include, but are not limited to a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g., pentoxifylline) and bupropion. An example of an IL-6 inhibitor is an anti-IL-6 antibody molecule such as tocilizumab (toe), sarilumab, elsilimomab, CNTO 328, ALD518/BMS-945429, CNTO 136, CPSI-2364, CDP6038, VX:30, ARGX-109, FE301, and FMI 01. In some embodiments, the anti-IL-6 antibody molecule is tocilizumab. An example of an IL-IR based inhibitor is anakinra.

In some embodiment, the subject is administered a corticosteroid, such as, e.g., methylprednisolone, hydrocortisone, among others.

In some embodiments, the subject is administered a vasopressor, such as, e.g., norepinephrine, dopamine, phenylephrine, epinephrine, vasopressin, or a combination thereof.

In some embodiments, the subject can be administered an antipyretic agent. In some embodiments, the subject can be administered an analgesic agent.

In some embodiments, the subject can be administered an agent which enhances the activity of the cell. For example, in some embodiments, the agent can be an agent which inhibits an inhibitory molecule. Inhibitory molecules, e.g., Programmed Death 1 (PD1), can, in some embodiments, decrease the ability of the cell to mount an immune effector response. Examples of inhibitory molecules include PD1, PD-L1, CTLA4, TIM3, CEACAM, (e.g., CEACAM-1, CEACAM-3 and/or CEACAM-5), LAG3, VISTA, BTLA, TIGIT, LAIRI, CD160, 2B4 and TGF beta. Inhibition of an inhibitory molecule, e.g., by inhibition at the DNA, RNA or protein level, can optimize a CAR-expressing cell performance. In some embodiments, an inhibitory nucleic acid, e.g., an inhibitory nucleic acid, e.g., a dsRNA, e.g., an siRNA or shRNA, or a clustered regularly interspaced short palindromic repeats (CRISPR), a transcription-activator-like effector nuclease (TALEN), or a zinc finger endonuclease (ZFN), can be used to inhibit expression of an inhibitory molecule in the CAR-expressing cell. In some embodiments the inhibitor is an shRNA. In some embodiments, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR. In some embodiments, the inhibitor of an inhibitory signal can be, e.g., an antibody or antibody fragment that binds to an inhibitory molecule. For example, the agent can be an antibody or antibody fragment that binds to PDI, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In some embodiments, the agent is an antibody or antibody fragment that binds to TIM3. In some embodiments, the agent is an antibody or antibody fragment that binds to LAG3. In some embodiments, the agent is an antibody or antibody fragment that binds to CEACAM (e.g., CEACAM-1, CEACAM-3 and/or CEACAM-5).

PD1 is an inhibitory member of the CD28 family of receptors that also includes CD28, CTLA-4, ICOS, and BTLA. PD1 is expressed on activated B cells, T cells and myeloid cells (Agata et al. 1996 Int. Immunol 8:765-75). Two ligands for PDI, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol. 32:634-43).

PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-LI. Antibodies, antibody fragments, and other inhibitors of PDI, PD-L1 and PD-L2 are available in the art and may be used combination with a CAR described herein. For example, nivolumab (also referred to as BMS-936558 or MDXI 1 06; Bristol-Myers Squibb) is a fully human IgG4 monoclonal antibody which specifically blocks PDI. Nivolumab (clone 5C4) and other human monoclonal antibodies that specifically bind to PD1 are disclosed in U.S. Pat. No. 8,008,449 and WO2006/121168. Pidilizumab (CT-011; Cure Tech) is a humanized IgGlk monoclonal antibody that binds to PDIPidilizumab and other humanized anti-PD 1 monoclonal antibodies are disclosed in WO2009/10161 1. Pembrolizumab (formerly known as lambrolizumab and also referred to as Keytruda, MK.03475; Merck) is a humanized IgG4 monoclonal antibody that binds to PDI. Pembrolizumab and other humanized anti-PD 1 antibodies are disclosed in U.S. Pat. No. 8,354,509 and WO2009/1 14335. MEDI4736 (Medimmune) is a human monoclonal antibody that binds to PDLI, and inhibits interaction of the ligand with PDI. MDPL3280A (Genentech I Roche) is a human Fe optimized IgG1 monoclonal antibody that binds to PD-LI. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs: 20 and 21 in WO2010/077634) and MDX-1 105 (also referred to as BMS-936559, and, e.g., anti-PD-L1 binding agents disclosed in WO2007/005874). AMP-224 (B7-DCig; Amplimmune; e.g., disclosed in WO2010/027827 and WO201 1/066342), is a PD-L2 Fe fusion soluble receptor that blocks the interaction between PD1 and B7-H1. Other anti-PD 1 antibodies include AMP 514 (Amplimmune), among others, e.g., anti-PD 1 antibodies disclosed in U.S. Pat. No. 8,609,089, US 2010028330, and/or US 20120114649. TIM3 (T cell immunoglobulin-3) also negatively regulates T cell function, particularly in IFN-7-secreting CD4+ T helper 1 and CD8+ T cytotoxic 1 cells, and plays a critical role in T cell exhaustion. Inhibition of the interaction between TIM3 and its ligands, e.g., galectin-9 (Gal9), phosphotidylserine (PS), and HMGBI, can increase immune response. Antibodies, antibody fragments, and other inhibitors of TIM3 and its ligands are available in the art and may be used combination with a CAR, e.g., a described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-I are disclosed in US20130156774.

In some embodiments, the agent which enhances the activity of the cell is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. 2010 Sep. 2; 5(9). pii: e125295 (DOI:I0:1371/journal.pone.0021146), or cross reacts with CEACAM-1 and CEACAM-5 as described in, e.g., WO 2013/054331 and US 2014/0271618.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with fusion protein(s) or KIR CAR herein. For example, BMS-986016 (Bristol-Myers Squib) is a monoclonal antibody that targets LAG3. IMP701 (Immutep) is an antagonist LAG3 antibody and IMP73 1 (Immutep and GlaxoSmithKline) is a depleting LAG3 antibody. Other LAG3 inhibitors include IMP321 (Immutep), which is a recombinant fusion protein of a soluble portion of LAG3 and 1 g that binds to MHC class II molecules and activates antigen presenting cells (APC). Other antibodies are disclosed, e.g., in WO2010/019570.

In some embodiments, the agent which enhances the activity of the cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a 10 positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In some embodiments, the fusion protein is expressed by the same cell that expressed the CAR. In some embodiments, the agent which enhances activity of the cell is miR-17-92.

### Dosing Regimen

Described above are methods of treating. In some embodiments, a dosage of cells is based on cell/kg body weight and the total cells in the pharmaceutical composition. In some embodiments, the total cells measured herein do not include cells received by the patient prior to the cell treatment.

In some embodiments, a pharmaceutical composition comprising the cell(s), e.g., T cells or NK cells, comprising a nucleic acid molecule encoding a fusion protein(s) can be administered, e.g., intravenously, at a dosage of about 3×10⁴ cells/kg body weight for patients >50 kg body weight or about 1.5×10⁶ cells for patients >50 kg body weight based on the total cells in the pharmaceutical composition. In some embodiments, a patient with >50 kg body weight is between 1 and 30 years of age.

In some embodiments, the pharmaceutical composition is administered in a single infusion, e.g., via intravenous administration, at the dosages as described throughout. In some embodiments, the pharmaceutical composition is administered multiple times, e.g., via intravenous administration, at the dosages described throughout. The cells can be administered by using infusion techniques that are commonly known in immunotherapy (see, e.g., Rosenberg et al., New Eng. J. of Med. 319:1676, 1988).

In certain embodiments, it may be desired to administer activated cells, e.g., T cells or NK cells, to a subject and then subsequently redraw blood (or have an apheresis performed), activate the cells therefrom according to the present disclosure, and reinfuse the patient with these activated and expanded cells. This process can be carried out multiple times every few weeks. In certain embodiments, cells, e.g., T cells or NK cells, can be activated from blood draws of from 10 cc to 400 cc. In certain embodiments, cells, e.g., T cells or NK cells, are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The pharmaceutical compositions described herein may be administered to a patient trans arterially, subcutaneously, intradermally, intratumorally, intranodally, intramedullary, intramuscularly, intracranially, by spinal injection, by intravenous (i.v.) injection, e.g., i.v. push and/or i.v. infusion, or intraperitoneally. In some embodiments, the cell comprising pharmaceutical compositions, e.g., T cell or NK cell compositions, of the present disclosure are administered to a patient by intradermal or subcutaneous injection. In some embodiments, the cell comprising pharmaceutical compositions, e.g., T cell or NK cell compositions, of the present disclosure are administered by i.v. injection. The compositions of cells, e.g., T cell or NK cell compositions, may be injected directly into a tumor, lymph node, or site of infection.

In a particular exemplary embodiment, subjects may undergo leukapheresis, wherein leukocytes are collected, enriched, or depleted ex vivo to select and/or isolate the cells of interest, e.g., TorNK cells. These cell isolates, e.g., T cell or NK cell isolates, may be expanded by methods known in the art and treated such that one or more constructs herein may be introduced, thereby creating a fusion protein(s) or KIR CAR-expressing cell. Subjects in need thereof may subsequently undergo standard treatment with high dose chemotherapy followed by peripheral blood stem cell transplantation. In certain embodiments, following or concurrent with the transplant, subjects receive an infusion of the expanded CAR-expressing cells of the present disclosure. In an additional embodiment, expanded cells are administered before or following surgery.

In some embodiments, lymphodepletion is performed on a subject, e.g., prior to administering one or more cells that express a CAR described herein. In some embodiments, the lymphodepletion comprises administering one or more of melphalan, cytoxan, cyclophosphamide, and fludarabine. In some embodiments, the lymphodepletion comprises administering cytarabine and/or etoposide.

The dosage of the above treatments to be administered to a patient will vary with the precise nature of the condition being treated and the recipient of the treatment. The scaling of dosages for human administration can be performed according to art-accepted practices. The dose for a therapeutic, e.g., an antibody, e.g., CAMPATH, for example, may generally be, e.g., in the range 1 to about 100 mg for an adult patient, e.g., administered daily for a period between 1 and 30 days. A suitable daily dose is 1 to 10 mg per day although in some instances, larger doses of up to 40 mg per day may be used (described in U.S. Pat. No. 6,120,766).

In some embodiments, the fusion protein(s) or KIR CAR is introduced into cells, e.g., T cells or NK cells, e.g., using in vitro transcription, and the subject (e.g., human) receives an initial administration of fusion protein(s) or KIR CAR-expressing cells, and one or more subsequent administrations of the cells, wherein the one or more subsequent administrations are administered less than 15 days, e.g., 14, 13, 12, 11, 10, 9, 8, 7, 6, 5, 4, 3, or 2 days after the previous administration. In some embodiments, more than one administration of the cells are administered to the subject (e.g., human) per week, e.g., 2, 3, or 4 administrations of the cells are administered per week In some embodiments, the subject (e.g., human subject) receives more than one administration of the cells per week or 2, 3, or 4 administrations per week (also referred to herein as a cycle), followed by a week of no cells, and then one or more additional administration of the the cells is administered to the subject. In some embodiments, the subject (e.g., human subject) receives more than one cycle of cells, and the time between each cycle is less than 10, 9, 8, 7, 6, 5, 4, or 3 days. In some embodiments, the cells are administered every other day for 3 administrations per week. In some embodiments, the cells are administered for at least two, three, four, five, six, seven, eight or more weeks.

In some embodiments, subjects may be adult subjects (i.e., 18 years of age and older). In certain embodiments, subjects may be between 1 and 30 years of age. In some embodiments, the subjects are 16 years of age or older. In certain embodiments, the subjects are between 16 and 30 years of age. In some embodiments, the subjects are child subjects (i.e., between 1 and 18 years. 10 of age).

In some embodiments, the cells are generated using lentiviral viral vectors, such as lentivirus. The cells generated that way will have stable fusion protein(s) or KIR CAR expression. In some embodiments, fusion protein(s) or KIR CAR-expressing cells transiently express a vector herein for 4, 5, 6, 7, 8, 9, 10, 11, 12, 13, 14, or 15 days after transduction. Transient expression can be effected by RNA vector delivery. In some embodiments, the RNA encoding a fusion protein(s) or KIR CAR is transduced into the cell, e.g., NK cell or T cell, by electroporation.

A potential issue that can arise in patients being treated is anaphylaxis after multiple treatments. Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient therapy (such as those generated by RNA transductions), infusion breaks should not last more than ten to fourteen days.

In some embodiments, one or more cell herein can be administered or delivered to the subject via a biopolymer scaffold, e.g., a biopolymer implant. Biopolymer scaffolds can support or enhance the delivery, expansion, and/or dispersion of the cells herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-viral infected cell activity, of the cells to be delivered. The biopolymer scaffold can be an injectable, e.g., a gel or a semi-solid, or a solid composition.

In some embodiments, cells herein are seeded onto the biopolymer scaffold prior to delivery to the subject. In some embodiments, the biopolymer scaffold further comprises one or more additional therapeutic agents described herein (e.g., another CAR-expressing cell, an antibody, or a small molecule) or agents that enhance the activity of a fusion protein(s) or KIR CAR-expressing cell, e.g., incorporated or conjugated to the biopolymers of the scaffold. In some embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/1 10591.

### EXAMPLES

The below examples and the associated figures refer to dual binders DB1, DB2, DB3, DB4, DB5, DB6, DB7, and DB8. The fusion proteins of Table 11 are DB1 through DB8. For comparison, Table 14 shows the names of the dual binders using the names and SEQ ID NOS of Table 11.

For all of the foregoing Examples, reference to “KIRShort” refers to KIR2DS2 short extracellular domain. When used in combination with an anti-CD19 or anti-CD20 binder, it also includes a (G4S)2 linker. Further, reference to “KIRLong” refers to KIR2DS2 long extracellular domain. When used in combination with an anti-CD19 or anti-CD20 binder, it also includes a (G4S)5 linker.

TABLE 14

| SEQ ID. NO. | Name | Dual Binder Name |
|---|---|---|
| 681 | 1-T19_P20 | DB1 |
| 682 | 2-T20_P19 | DB2 |
| 683 | 3-P20_P19 | DB3 |
| 684 | 4-P19_P20 | DB4 |
| 685 | 5-T20_T19 | DB5 |
| 686 | 6-T19_T20 | DB6 |
| 687 | 7-P20_T19 | DB7 |
| 688 | 8-P19_T20 | DB8 |

### Example 1—Functionality of Human T Cells Electroporated with mRNA Expressing Dual KIR-CARs

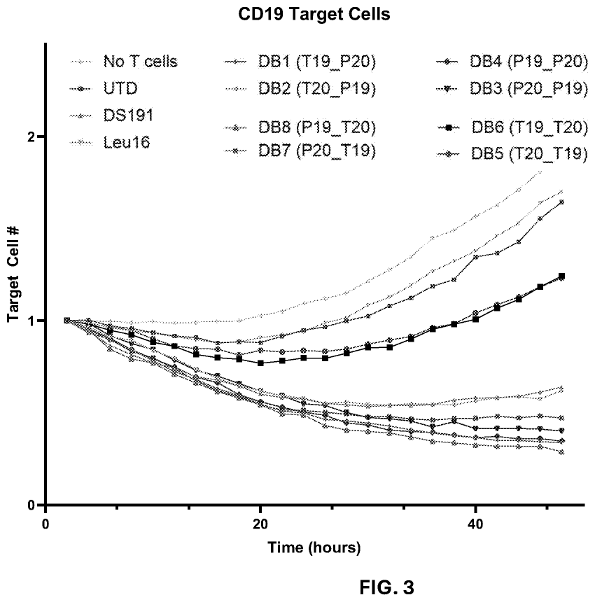

FIG. 3 shows that all dual binder candidates are effective at killing CD19+ target cells. 120,000 T cells cocultured with 40,000 target cells (K562-CD19 GFP) at a 3:1 E:T ratio.

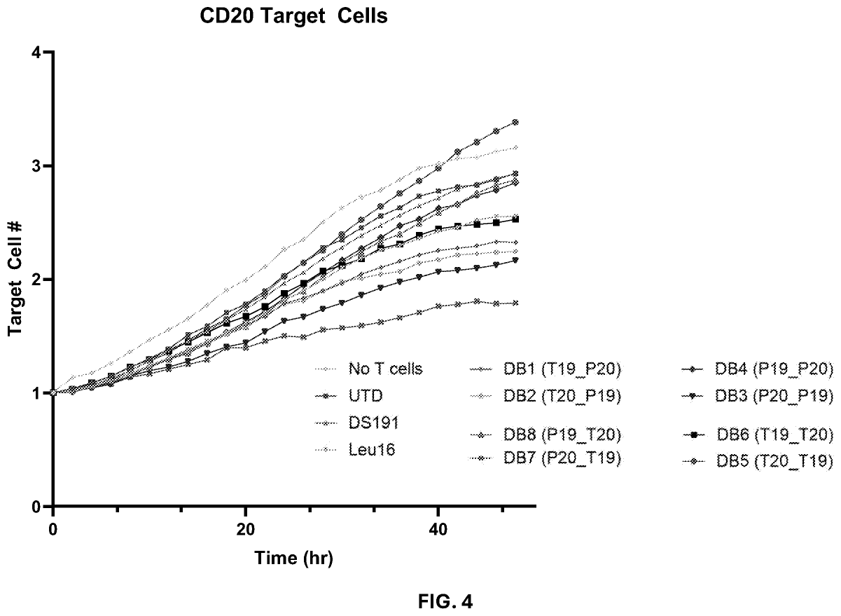

FIG. 4 shows limited efficacy of dual binder candidates at killing CD20+ target cells when mRNA electroporation is used to induce expression. 40,000 target cells (K562-CD20 GFP) at a 3:1 E:T ratio.

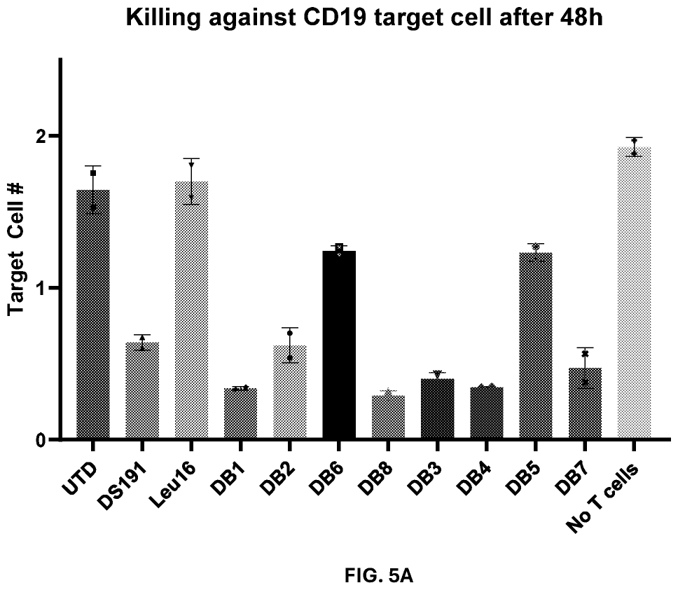

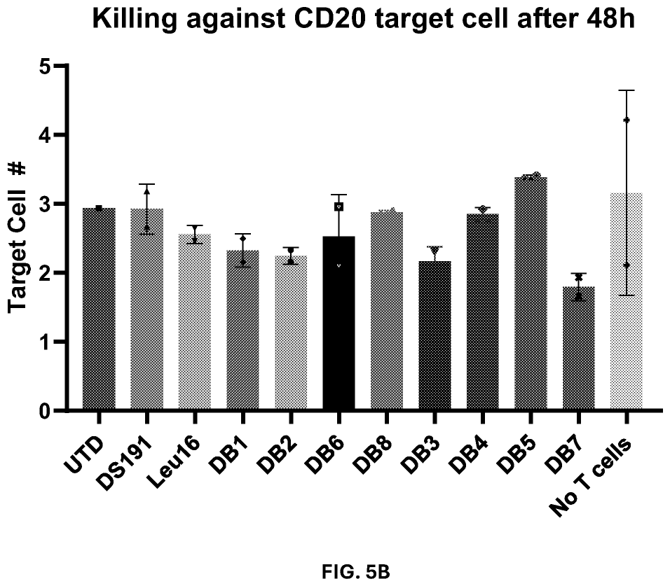

FIG. 5 shows overall killing of CFD19 and CD20 target cells with mRNA electroporation of dual KIR-CAR into T cells.

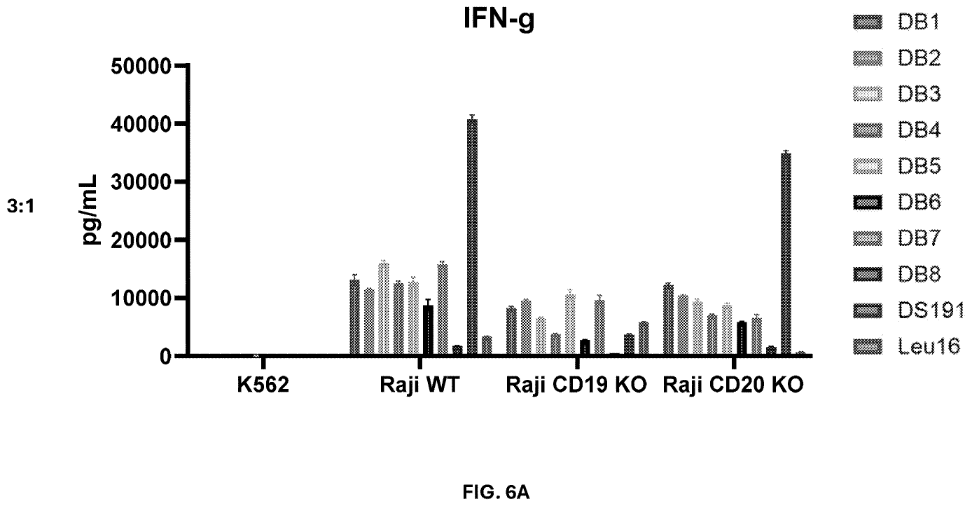

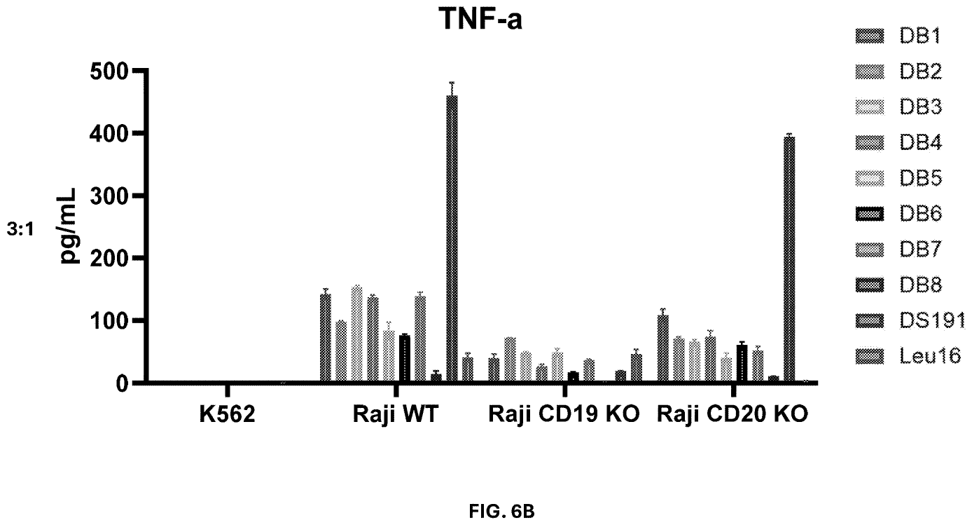

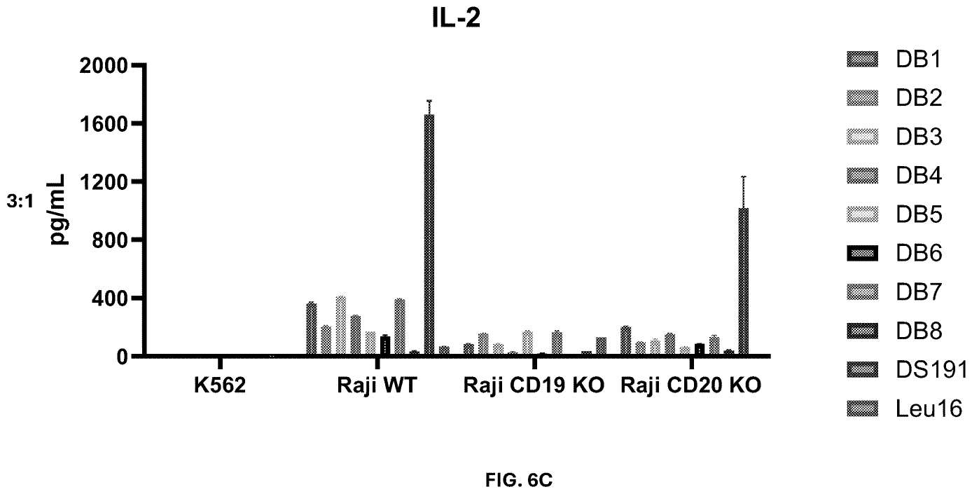

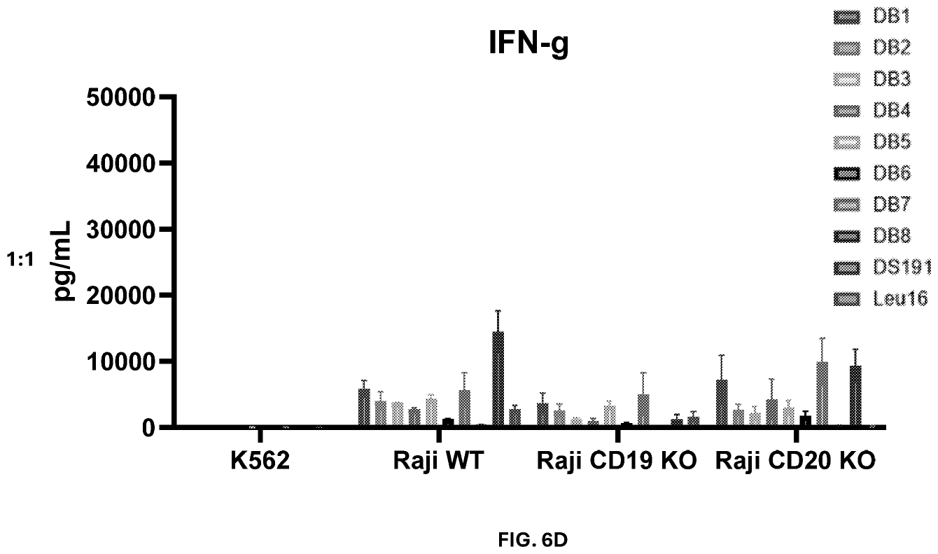

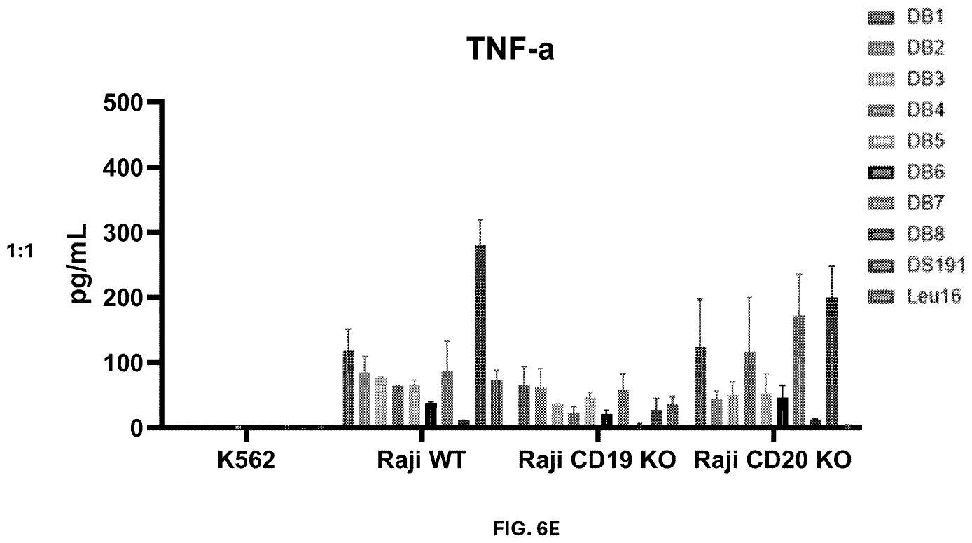

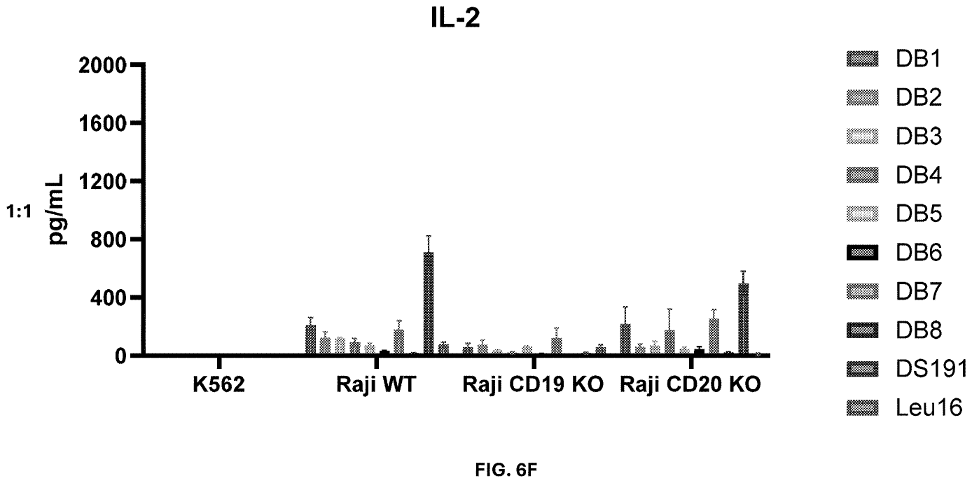

FIG. 6 shows cytokine secretion after 24 hour co-culture with CD19/20 dual-positive, CD19+/CD20− or CD19−/CD20+ Raji target cells.

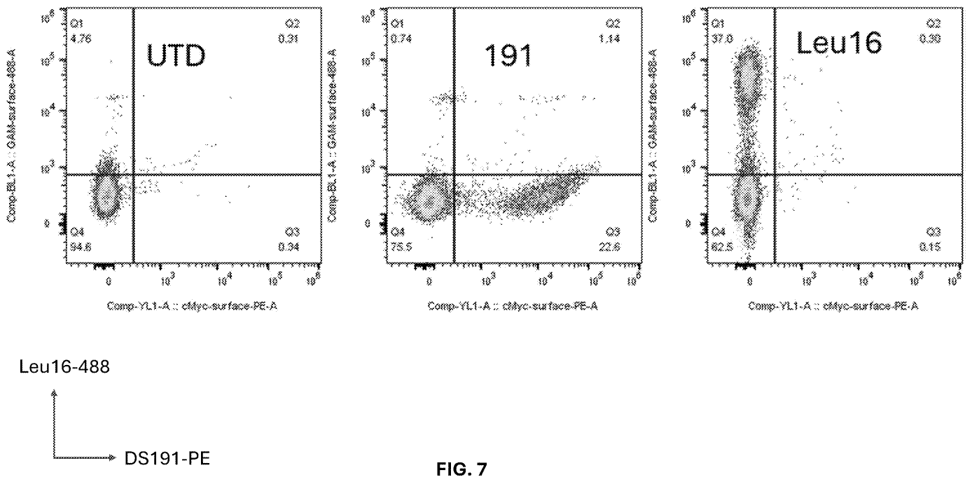

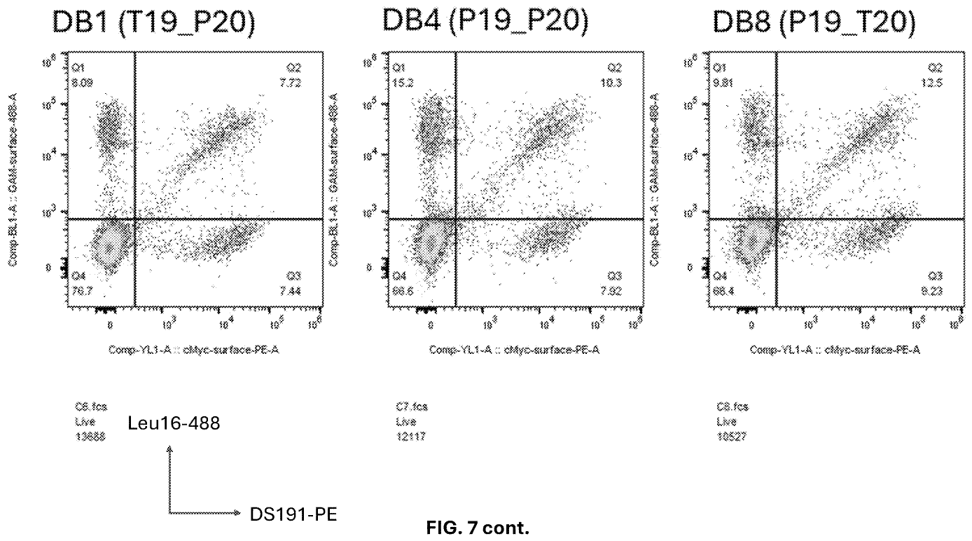

FIG. 7 shows surface expression of dual KIR-CAR in T cells after lentivirus transduction (Donor 1).

### Example 2—Testing Functionality Using Lentivirus of Select Dual Binder Candidates (DB1, DB4, and DB8)

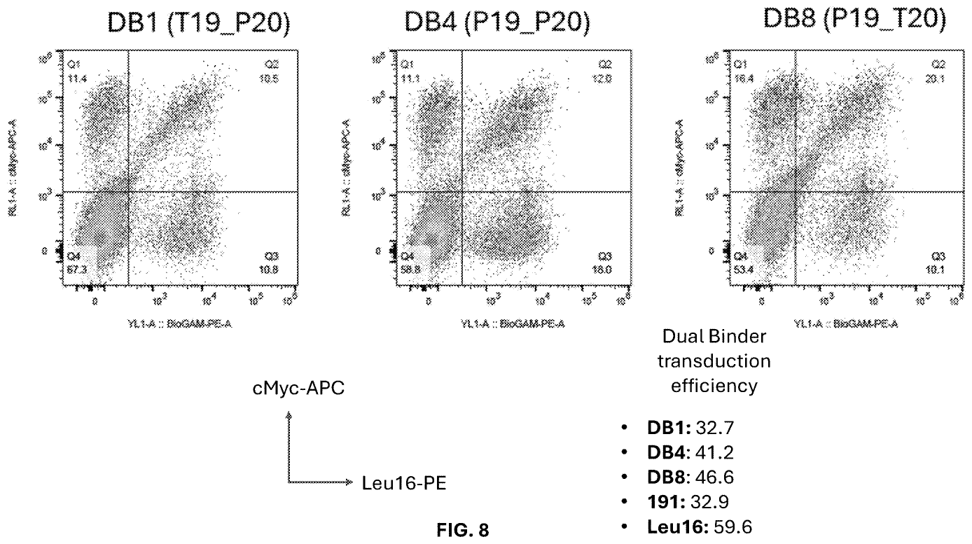

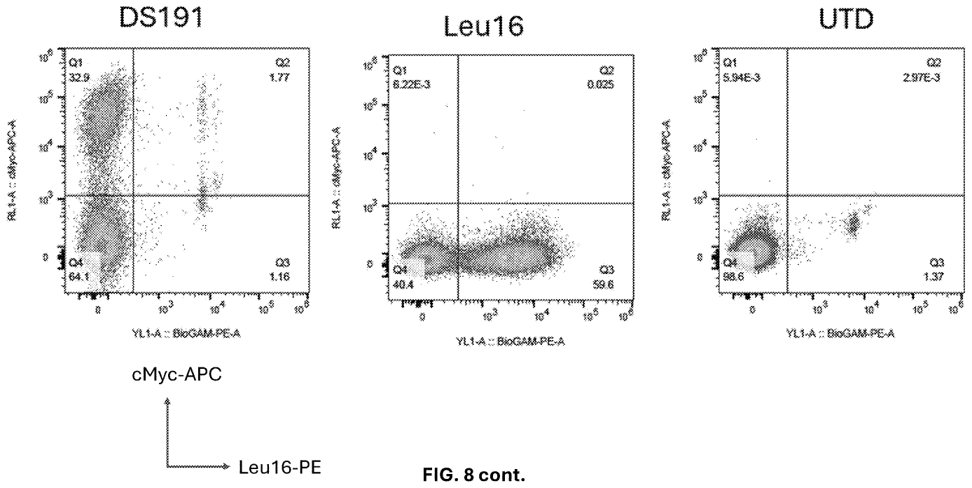

FIG. 8 shows surface expression of binders in T cells after lentivirus transduction (Donor 2).

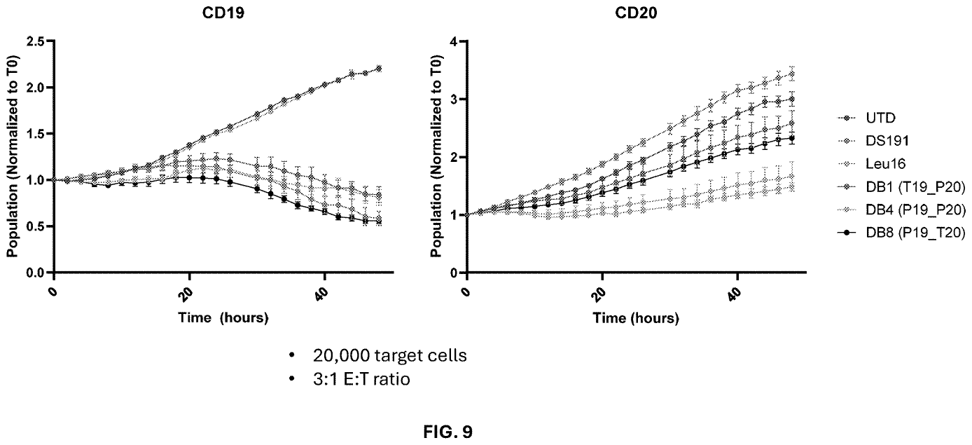

FIG. 9 illustrates donor 1 T cells mediated antigen-specific killing. Dual binder candidates effectively kill both CD19+ and CD20+ target cells. 20,000 target cells at a 3:1 E:T ratio.

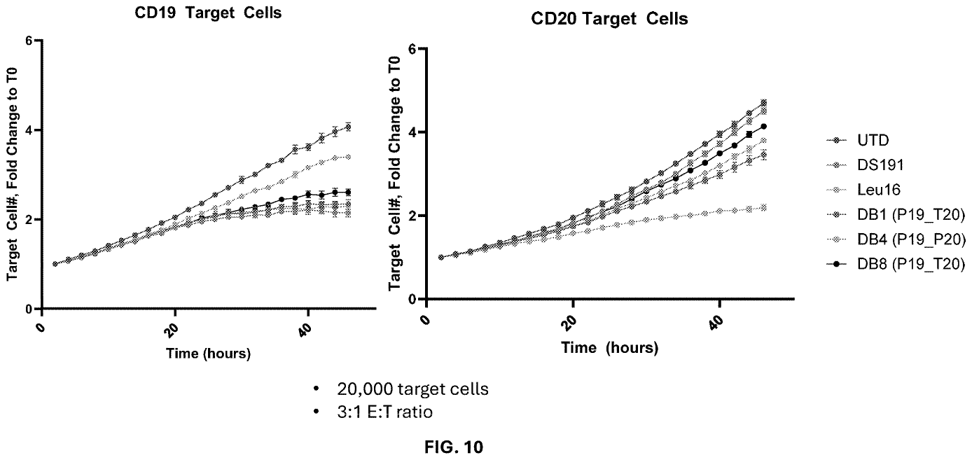

FIG. 10 FIG. 9 illustrates donor 1 killing. Dual binder candidates effectively kill both CD19+ and CD20+ target cells. 20,000 target cells at a 3:1 E:T ratio.

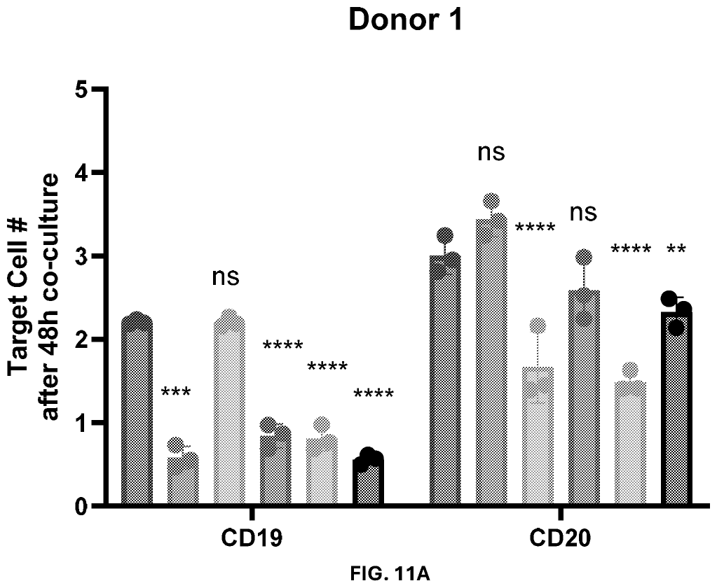

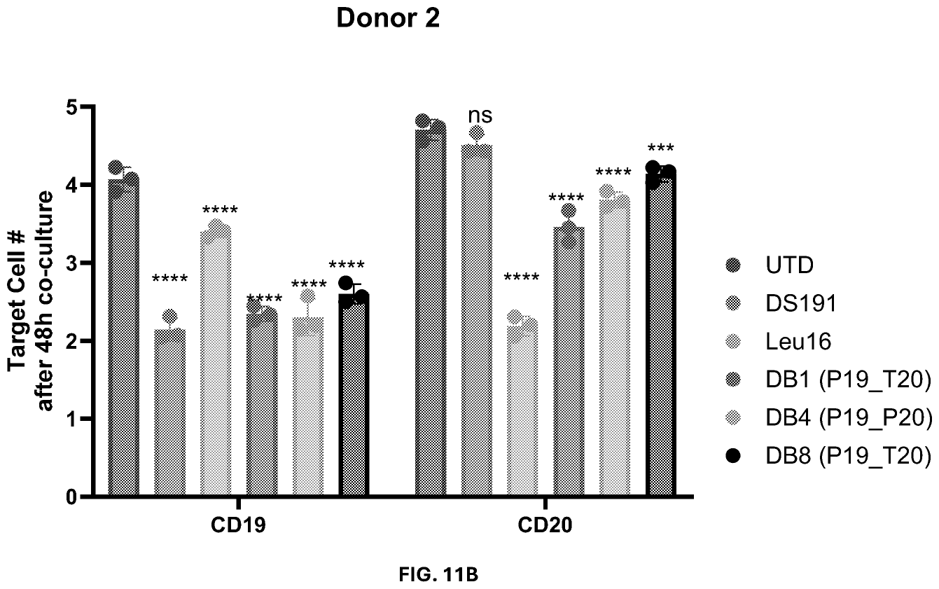

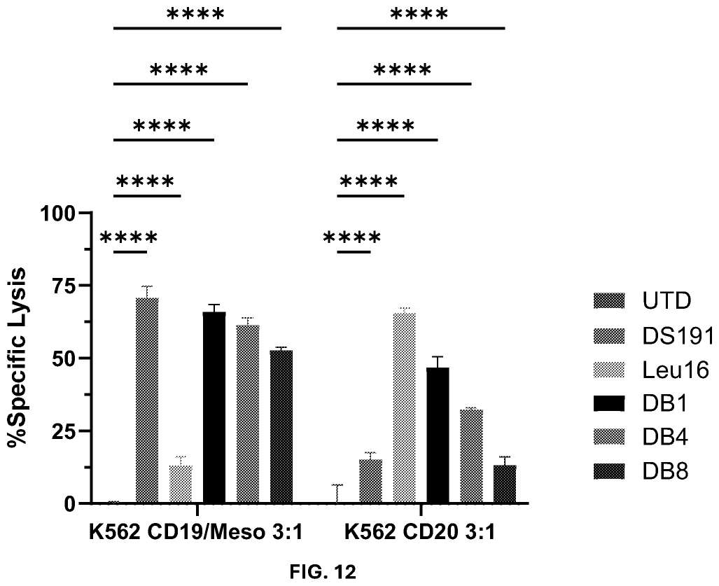

FIG. 11 shows that dual-KIR effectively kills both CD19+ and CD20+ target cells (calculated by normalized target cell total number after 48 hours of co-culture). FIG. 12 shows target cell kill calculated by specific lysis with CD19+ or CD20+ targets.

### Example 3—Prophetic Demonstration of on Target Efficacy of Tricistronic DS191 (Targeting CD19) and Leu16 (Targeting CD20) Directed Dual KIR-CAR T Cells Both In Vitro and In Vivo

### In Vitro Experiments

Transduction efficiency will be evaluated via the transduction efficiency of DS191- and Leu-directed KIR-CAR in healthy donor T cells.

DAP12 staining: Expression of intracellular DAP12 will be evaluated through anti-DAP12 staining to confirm expression overlap with DS191- and Leu16-positive populations in healthy donor T cells.

Killing assay: Assess The cell killing abilities of DS191- and Leu16-directed KIR-CAR T cells will be assessed by co-cultured with CD19-positive and CD20-positive target cells compared to off-target controls using an Incucyte or luciferase-based assay.

Cytokine secretion: The concentration of cytokines secreted by DS191- and Leu16-directed KIR-CAR T cells will be assessed after co-culture with CD19- and CD20-positive target cells compared to off-target controls.

### In Vivo Experiments

On target-killing: Evaluate The ability of DS191- and Leu16-directed KIR-CAR T cells to target and kill CD19- and CD20-positive target cells in vivo will be evaluated. NSG mice will be injected with target tumor cells prior to injection with DS191- and Leu16-directed KIR-CAR T cells and tumor cell killing will be monitored over time using Bioluminescence imaging (BLI). Mice injected with untransduced T cells will be used as a negative control.

Compare efficacy of top candidates: The efficacy of the top DS191- and Leu16-directed KIR-CAR T cell candidates will be compared in vivo. Two top candidates will be selected after completion of the above in vitro experiments. Selected DS191- and Leu6-directed KIR-CAR T cell will be injected into NSGmice that were previously injected with CD19- and CD2-positive target tumor cells and tumor cell killing will be monitored over time using Bioluminescence imaging (BLI). Mice injected with untransduced T cells will be used as a negative control.

### Example 4—Prophetic Examples Over Fusion Proteins 9-24 of Table 8

The fusion proteins 9-24 are represented in Table 15, below, to highlight the elements of each.

TABLE 15

| Fusion | |
|---|---|
| Protein | Elements |
| 9 | DS191-KIRshort |
| 10 | Leu16-(G4S)₂-KIRshort |
| 11 | Leu16-(G4S)₅-KIRshort |
| 12 | Leu16-KIRlong (including (G4S)₅ linker) |
| 13 | DAP12-T2A-DS191-KIRshort-P2A-Leu16-KIRshort |
| 14 | DAP12-T2A-Leu16-KIRshort-P2A-DS191-KIRshort |
| 15 | DAP12-P2A-Leu16-KIRshort-P2A-DS191-KIRshort |
| 16 | DAP12-P2A-DS191-KIRshort-P2A-Leu16-KIRshort |
| 17 | DAP12-T2A-Leu16-KIRshort-T2A-DS191-KIRshort |
| 18 | DAP12-T2A-DS191-KIRshort-T2A-Leu16-KIRshort |
| 19 | DAP12-P2A-Leu16-KIRshort-T2A-DS191-KIRshort |
| 20 | DAP12-P2A-DS191-KIRshort-T2A-Leu16-KIRshort |
| 21 | DAP12-T2A-DS191-KIRshort-P2A-Leu16-KIRlong |
| | (including G4S)₅ linker)) |
| 22 | DAP12-P2A-DS191-KIRshort-T2A-Leu16-KIRlong |
| 23 | DAP12-T2A-Leu16-KIRlong-P2A-DS191-KIRshort |
| 24 | DAP12-P2A-Leu16-KIRlong-T2A-DS191-KIRshort |

Example 4.1: Evaluate the transduction efficiency of healthy donor T cells electroporated with mRNAs for different KIR-CARs including CD19-specific DS191-KIR, CD20-specific Leu16-KIR, and various versions of dual KIR-CARs (fusion proteins #13-24).

Expected result: Dual KIR-CAR T cells after mRNA electroporation will show good transfection efficiency, characterized by DS191 and Leu16 positivity. Comparable transfection efficiency to single DS191 or Leu16 KIR-CARs.

Method: Primary human T cells from healthy volunteer donors will be activated using anti-CD3/anti-CD28 coated Dynabeads (ThermoFisher) at a bead:cell ratio of 3:1 and cultured in OpTmizer (ThermoFisher) with 10% fetal bovine serum (FBS, Gemini Bio), 2 mM GlutaMax (ThermoFisher) supplemented with cytokines 5 ng/mL of IL-7 and IL-15 (Miltenyi Biotec). T cells will be expanded and measured every other day by Countess 3 (Thermo Fisher) for 8 days. After beads removal, T cells are formulated with cryomedia containing 10% DMSO. Cryopreserved T cells are thawed and washed with fresh media. In vitro transcription (IVT) RNA for different KIR-CAR will be prepared using the T7 mScript messenger RNA production system (Cellscript). Cryopreserved T cells were thawed and rested overnight. T cells will be electroporated with 6.4 ug IVT RNA for different KIR-CAR per 2×10⁵ cells using the Neon electroporation system (ThermoFisher). After 24 hours, CAR expression will be detected on electroporated T cells by flow cytometry prior to coculture with target cells. T cells will be stained with FACS buffer containing antibodies: Alx647-conjugated anti-mycTAG (Cell Signaling), and R-phycoerythrin (PE) conjugated goat-anti-mouse Fab (Jackson ImmunoResearch). mycTAG staining is to detect DS191-KIR receptor expression. Goat-anti-mouse Fab staining is to detect Leu16-KIR receptor expression. A fixable viability dye (ThermoFisher) is used to gate on viable cells. 20 min later, T cells are washed with FACS buffer twice and resuspended with 300 uL FACS buffer for acquisition on the flow cytometer Thermo Fisher Attune.

Data will be presented as a set of flow cytometry plots showing CAR receptor expression in human T cells electroporated with different in vitro transcription mRNA for different dual KIR-CARs.

Example 4.2: assess the tumor killing abilities of single DS191-KIR, Leu16-KIR, and different versions of dual KIR-CAR T cells. T cells will be first electroporated with mRNAs for different KIR-CAR constructs (constructs #13-20) for 24 hours and then will be co-cultured with luciferase-expressing target cells including wildtype K562 and K562 cells genetically engineered to express CD19 (CD19+ target cells) or CD20 (CD20+ target cells), using the Incucyte (Sartorius) live-cell imaging system.

Expected result: Differential dual KIR-CAR T cells by mRNA electroporation will show specific tumor killing against CD19+ or CD20+ targets.

Method: Once transfection efficiency is determined in T cells after mRNA electroporation, KIR-CAR T cells will be cocultured with target cells expressing CD19 or CD20 cells and cytotoxicity will be determined using the Incucyte. K562 target cells will be attached to poly-L-ornithine coated (Sigma-Aldrich), 96-well flat-bottom dishes at a concentration of 20,000 cells/well. mRNA electroporated T cells will be added at a 3:1 Effector:Target cell ratio in cytokine-free media with 3 replicates per target (K562-CD19 or K562-CD20) and KIR-CAR T cell combination. Three images will be acquired per well at 20× magnification every 2 hr for 48 h, and cell number quantified using the Sartorius Incucyte Cell Proliferation Analysis Software. Killing will be calculated at each time point by normalization to cell number at time 0, and subtracting this fold change from the fold change in target cell number in the UTD condition according to the formula below:

UTDn=(Target cell number for UTD time point N)/(Target cell number for UTD time point 0)

KIRn=(Target cell number for KIR CAR time point N)/(Target cell number for KIR CAR time point 0)

Killing at timepoint N=(UTDn−KIRn)/(UTDn)

Data will be presented as a set of plots showing dual KIR-CAR T cells mediate tumor Killing in an antigen-specific manner.

Examples 4.3: evaluate the transduction efficiency of healthy donor T cells transduced with lentiviral vectors expressing different CAR constructs including CD19-specific DS191-KIR, CD20-specific Leu16-KIR, and various versions of dual KIR-CARs. One of the panels to show no recombination events occur in the optimized dual KIR-CAR constructs compared to non-optimized dual KIR-CAR constructs.

Expected result: Dual KIR-CAR T cells show a good transduction efficiency, characterized by 20-50% DS191 and Leu16 positivity. High correlation between DS191 and Leu16 KIR-CAR expression in the dual KIR-CAR LV transduced T cells. Comparable transduction efficiency to single DS191 or Leu16 KIR-CARs.

Method: Primary human T cells from healthy volunteer donors will be activated using anti-CD3/anti-CD28 coated Dynabeads (ThermoFisher) at a bead:cell ratio of 3:1 and cultured in OpTmizer (Thermo Fisher) with 10% fetal bovine serum (FBS, Gemini Bio), 2 mM GlutaMax (ThermoFisher) supplemented with cytokines 5 ng/mL of IL-7 and IL-15 (Miltenyi Biotec). 24 hrs after activation, the T cells will be transduced with lentiviruses targeting 50% transduction efficiency. T cells will be then expanded and measured every other day by Countess 3 (Thermo Fisher) for 3-10 days. After beads removal, T cells are formulated with cryomedia containing 10% DMSO and frozen for functional testing (in vitro tumor killing, cytokine production, and in vivo animal studies). Fresh T cells are harvested and stained with FACS buffer containing antibodies: Alx647-conjugated anti-mycTAG (Cell Signaling), and R-phycoerythrin (PE) conjugated goat-anti-mouse Fab (Jackson ImmunoResearch). mycTAG staining is to detect DS191-KIR receptor expression. Goat-anti-mouse Fab staining is to detect Leu16-KIR receptor expression. A fixable viability dye is used to gate on viable cells. 20 min later, T cells are washed with FACS buffer twice and resuspended with 300 uL FACS buffer for acquisition on the flow cytometer Thermo Fisher Attune or Agilent Penteon.

Data will be presented as a set of flow cytometry plots showing CAR receptor expression of different dual KIR-CAR constructs in human T cells.

Example 4.4: to evaluate expression of intracellular DAP12 through anti-DAP12 staining in healthy donor T cells transduced with lentiviral vectors expressing different KIR-CAR constructs including CD19-specific DS191-KIR, CD29-specific Leu16-KIR, and various versions of dual KIR-CARs.

Expected result: DAP12 is highly expressed in dual KIR-CAR T cells. Comparable DAP12 expression in single DS191 or Leu16 KIR-CARs.

Method: Fresh T cells are harvested and stained with FACS buffer containing antibodies Alx647-conjugated anti-DAP12 (BD). A fixable viability dye is used to gate on viable cells. 20 min later, T cells are washed with FACS buffer twice and resuspended with 300 uL FACS buffer for acquisition on the flow cytometer Thermo Fisher Attune or Agilent Penteon.

Data will be presented as flow cytometry plots showing intracellular DAP12 expression of CD19-specific DS191-KIR, CD20-specific Leu16-KIR, and various versions of KIR-CAR constructs.

Example 4.5: To assess the tumor killing abilities of single DS191-KIR, Leu16-KIR, and different versions of dual KIR-CAR T cells co-cultured with luciferase-expressing target cells including CD19+CD20+ Raji (wildtype), CD19+CD20− (CD19+CD20knockout/CD19+CD20KO) Raji, CD19-CD20+ (CD19knockoutCD20+/CD19KOCD20+) Raji, and K562 genetically engineered to express CD19 (K562-OE CD19) or CD20 (K562-OE CD20), compared to CD19-negative CD20-negative controls (K562 wildtype) using an Incucyte (Sartorius) or luciferase-based assay (Promega).

Expected result: At least comparable tumor killing to single DS191 or Leu16 KIR-CARs.

### Methods

Raji and K562 target cell lines will be generated by first infecting wild-type cell lines with a lentivirus encoding GFP and click-beetle luciferase (CBG). Cells will be allowed to expand following infection, and sorted 2 times for the upper 25% of GFP expression using the Tyto Cell Sorter (Miltenyi Biotec) to achieve >99% purity. Cells will be monitored by flow cytometry for to ensure stable GFP expression for >4 weeks.

To generate CD20 overexpression K562 cell lines (K562-OE CD20), a lentivirus encoding humans CD20 will be used to infect K562-CBG-GFP expressing lines at a MOI of 10. Following infection and expansion, cells will be stained for CD20 and sorted using the Tyto Cell Sorter. To generate Raji knockout cell lines, guide sgRNAs (Thermo Fisher) targeting CD19 or CD20 will be electroporated along with Cas9 protein (Invitrogen) using the Neon N×T Electroporation System (Thermo Fisher Scientific). Knockout cells will be confirmed by staining with anti-CD19 or CD20 antibodies and negative cell lines will be sorted using Tyto cell sorter. The knockout cell lines are expanded and periodically tested for the absence of CD19 or CD20 by flow cytometry.

For antigen-specific killing assays, target cells and untransduced (UTD) or KIR-CAR+ T cells will be co-cultured at effector to target cell ratios of 3:1 and 1:1 for 48 h in 96-well dishes. For comparison between KIR-CAR constructs, transduced T cells will be normalized to the same transduction efficiency by addition of UTD cells to achieve equivalent transduction percentage prior to co-culture.

For quantitation of cell killing by Incucyte live cell imaging, K562 target cells will be attached to poly-L-ornithine coated (Sigma-Aldrich), 96-well flat-bottom dishes at a concentration of 20,000 cells/well. KIR-CAR T cells will be added at a 3:1 Effector:Target cell ratio in cytokine-free media with 3 replicates per target and KIR-CAR T cell combination. Three images will be acquired per well at 20× magnification every 2 hr for 48 h, and cell number quantified using the Sartorius Incucyte Cell Proliferation Analysis Software. Killing will be calculated at each time point by normalization to cell number at time 0, and subtracting this fold change from the fold change in target cell number in the UTD condition according to the formula below:

UTDn=(Target cell number for UTD time point N)/(Target cell number for UTD time point 0)

KIRn=(Target cell number for KIR CAR time point N)/(Target cell number for KIR CAR time point 0)

Killing at timepoint N=(UTDn−KIRn)/(UTDn)

For quantitation of cell killing by luciferase assay, 20,000 Raji or K562 target cells will be plated in 96-well round bottom dishes in 100 μL of cytokine-free media. KIR-CAR+ T cells will be added at 3:1 and 1:1 Effector:Target cell ratio in an additional 100 μL of cytokine-free media for a total volume of 200 uL. As a controls for maximum cell death (“Maximum Release”) target cells will be cultured with detergent (1% Triton X), and target cells will be cultured alone as a control for spontaneous cell death (“Spontaneous Release”). Cells will be co-cultured for 48 h in a 37 C, 5% CO2 incubator before being spun at 400 RPM to remove supernatants. After 1× wash in PBS, cell pellets will be lysed in 20 uL Luciferase Cell Lysis Buffer (Promega). Luciferase signal will be assayed using the Luciferase Assay System (Promega), and measured using the Varioskan Lux (Thermo Scientific). Specific lysis by KIR-CAR T cells will be calculated using the formula below:

% Specific Lysis=[(Experimental Release−Spontaneous Release)/(Maximum Release−Spontaneous Release)]*100.

Data will be presented as a set of plots showing dual KIR-CAR T cells mediate tumor Killing in an antigen-specific manner.

Example 4.6: Assess the cytokine production abilities of single DS191-KIR, Leu16-KIR, and dual KIR-CAR T cells co-cultured with target cells. Target cells assessed are CD19+CD20+ Raji (wildtype), CD19+CD20− (CD19+CD20knockout/CD19+CD20KO) Raji, CD19-CD20+ (CD19knockoutCD20+/CD19KOCD20+) Raji, and K562 genetically engineered to express CD19 (K562-OE CD19) or CD20 (K562-OE CD20), compared to off-target controls (K562 wildtype) using IFNg ELISA or MSD.

Expected result: Dual KIR-CAR T cells mediate cytokine production in an antigen-specific manner. At least comparable tumor killing to single DS191 or Leu16 KIR-CARs.

Methods: Target cells and untransduced (UTD) or KIR-CAR+ T cells as described above will be co-cultured at effector to target cell ratios of 3:1 and 1:1 for 48 h in 96-well round bottom dishes in a 5% CO₂, 37 C tissue culture incubator. For comparison between KIR-CAR constructs, transduced T cells will be normalized to the same transduction efficiency by addition of UTD prior to co-culture. Co-culture will be performed in cytokine-free media. After 24 h, co-cultures will be spun down to remove cells, and supernatants removed for cytokine quantitation. Concentrations of secreted cytokines (IL2, TNFα, IFNγ) will be quantified by immunoassay (Meso Scale Discovery).

### In Vivo Experimental Layout (June-November 2025)

Data will be presented as a set of plots showing dual KIR-CAR T cells mediate cytokine production in an antigen-specific manner.

Example 4.7: Assess the in vivo tumor killing abilities of KIR-CAR T cells including single binder DS191-KIR-CAR T cells, single binder Leu16-KIR-CAR T cells, and at least two different dual KIR-CAR T cells in immunocompromised NOD-scid IL2Rγc−/− (NSG) mice engrafted with CD19+CD20+ Raji (wildtype) tumors.

Expected result: Different versions of dual KIR-CAR T cells will demonstrate on-target killing in vivo that is at least comparable to killing by single binder DS191-KIR-CAR T cells or single binder Leu16-KIR-CAR T cells.

Experimental details: Tumor cells are engineered to express click beetle green (CBG) luciferase allowing for in vivo bioluminescence imaging (BLI). All NSG mice will first each be i.v. injected with 0.5 million target wildtype Raji tumor cells expressing CBG on Day 0 (prior to injection with KIR-CAR T cells). 5 days later, KIR-CAR T cells will be i.v. injected into tumor-bearing NSG mice at a dose of 0.5 million-2 million CAR+ T cells per mouse. The treatment groups will be as follows: untransduced (UTD) T cells as a negative control (n=8), DS191-KIR-CAR T cells (n=8), Leu16-KIR-CAR T cells (n=8), dual KIR-CAR T cells candidate #1 (n=8), dual KIR-CAR T cells candidate #2 (n=8). The tumor cell killing will be monitored over time by injecting mice with 150 mg/kg luciferin to activate the CBG followed shortly by BLI. Data will be plotted as the mean+/−SEM of the total flux (photons/second) of luminescent tumor cells per group at each time point. Data will be analyzed using one-way ANOVA with multiple comparisons.

FIG. 9: A set of plots showing dual KIR-CAR T cells can target and kill Raji tumor cells containing mixed populations (wildtype Raji and CD19KOCD20+ Raji).

Purpose: Assess the in vivo tumor killing abilities of KIR-CAR T cells including single binder DS191-KIR-CAR T cells, single binder Leu16-KIR-CAR T cells, and different dual KIR-CAR T cells in immunocompromised NSG mice engrafted with mixed Raji (wildtype plus CD19KOCD20+).

Expected result: Top candidate will demonstrate the highest anti-tumor activity against CD19KO CD20+ Raji in vivo.

Experimental Details: Tumor cells are engineered to express click beetle green (CBG) luciferase allowing for in vivo bioluminescence imaging (BLI). All NSG mice will first each be i.v. injected with 0.5 million target mixed Raji (wildtype plus CD19KOCD20+) tumor cells expressing CBG on Day 0 (prior to injection with KIR-CAR T cells). 5 days later, KIR-CAR T cells will be i.v. injected into tumor-bearing NSG mice at a dose of 0.5 million-2 million CAR+ T cells per mouse. The treatment groups will be as follows: untransduced (UTD) T cells as a negative control (n=8), DS191-KIR-CAR T cells (n=8), Leu16-KIR-CAR T cells (n=8), dual KIR-CAR T cells candidate #1 (n=8), dual KIR-CAR T cells candidate #2 (n=8). The tumor cell killing will be monitored overtime by injecting mice with 150 mg/kg luciferin to activate the CBG followed shortly by BLI. Data will be plotted as the mean+/−SEM of the total flux (photons/second) of luminescent tumor cells per group at each time point. Data will be analyzed using one-way ANOVA with multiple comparisons.

Data will be presented as a set of plots showing dual KIR-CAR T cells can target and kill CD19+CD20+ Raji wildtype tumor cells.

### Example 5—Materials and Methods for Examples 6-10

Construction of single and dual-KIR constructs. All plasmids were constructed in Verismo's preclinical laboratory and propagated using Endotoxin-free plasmid maxi kit (Qiagen, Cat #12362) prior to lentivirus production. The bicistronic CAR-T construct huCD19-28z-huCD20-41BBz transgene was generated based on sequences reported in Lam, et al (2021; Patent #US20210363245A1) and cloned into a lentiviral construct.

Generation of lentiviral vectors. Briefly, 293T cells were grown in RPMI 1640 containing 10% FBS and co-transfected with third-generation lentiviral vector transfer plasmids including all single and dual KIR constructs, as well as huCD19-28z-huCD20-41BBz along with pMDL.g/p, pRSV.rev and pVSVg packaging plasmids using lipofectamine 2000 transfection reagent. Vector-containing supernatants were harvested at 24 and 48 hours after transfection, and concentration was performed by Sorvall Lynx 6000 at 12,000 rpm at 4° C. for 18 hours. Concentrated vectors were stored at −80° C. until use. Vector titers were determined by a limiting dilution method on SUPT1 cells. Titrations were performed in 96-well flat-bottom microtiter plates containing 100 μL of activated T cells at 10⁶ cells per mL. Vectors were diluted by 3-fold serial dilution, and 50 mL of diluted vector supernatant was added to wells containing SUPT1 cells. The percentage of T cells expressing mycTAG was then determined by antibody staining (anti-myc Ab) and flow cytometry. The titer in transduction units per mL (TU/mL) for each vector was calculated from the vector dilution in which the percentage of positive cells was less than 20% but greater than 5% using the formula: Titer=% positive×dilution×10³×20.

Mice. The animal experiment protocols were approved by Charles River Accelerator and Development Lab (CRADL, Philadelphia) and the experiments were conducted in accordance with the Institutional Animal Care and Use Committee (IACUC). NOD-SCID-IL2Rγ_{c}^{-/-} (NSG) mice were purchased from Jackson Laboratory and maintained at CRADL. Animals were maintained according to IACUC-approved protocols. Both female and male mice that were 5-8 weeks of age were randomized into each experimental group.

T cell expansion and expression. Fresh isolated human T cells from healthy donors were obtained from the commercial vendors. T cells were activated using anti-CD3/anti-CD28 coated Dynabeads (CTS Dynabeads™ CD3/CD28; Gibco, Thermo Fisher Scientific) at a bead:cell ratio of 3:1 and cultured with clinical-grade media containing 5 ng/mL of IL-7 and 5 ng/mL of IL-15 cytokines (Miltenyi Biotec). 24 hours after activation, the T cells were transduced with lentiviruses. T cells were then expanded in complete T cell media containing IL-7 and IL-15 and measured every other day for 9-10 days, prior to cryopreservation. Untransduced T cells were used as negative control (UTD). T cells were stained with anti-mycAlx647 antibody and CAR expression was evaluated by flow cytometry (Attune NxT, Thermo Fischer). Flow cytometric analysis was carried out using FlowJo X 10.5.3 (Tree Star).

Target cell line generation. Wild-type K562 (Cat #CCL-243) and Raji (Cat #CCL-86) were originally obtained from the American Type Culture Collection (ATCC). Cells were expanded in R10 media containing RPMI 1640 (Gibco, Cat #21870-84) and 10% fetal bovine serum (FBS, Gemini Bio, Cat #100-106-500), and 1% GlutaMax (Gibco, Cat #35050061) at a low passage and tested for Mycoplasma using the mycostrip (InvivoGen, Cat #rep-mys-20) as per the manufacturer's instructions. Authentication of cell lines was performed by the Labcorp based on criteria established by the International Cell Line Authentication Committee. To generate target cell lines expressing click-beetle green luciferase and green fluorescence protein (K562-CBG-GFP and Raji-CBG-GFP), K562 and Raji cells were transduced with a lentiviral vector encoding CBG-T2A-GFP. The K562 and Raji cells expressing high levels of GFP were then sorted using Tyto (Miltenyi Biotec). Cell lines were confirmed to be >99% positive for GFP prior to downstream manipulations. After sorting for GFP, CD20-expressing K562 cell lines were generated by transduction with a CD20 overexpression lentivirus and again sorted for CD20 using the Tyto. To generate CD19 KO or CD20 KO CBG-GFP Raji cell lines were generated by CRISPR knockout and sorted for absence of either CD19 or CD20 on the Tyto. The identity of cell lines was ultimately confirmed by Short tandem repeat (STR) profiling, with a cutoff of >80%.

### In Vivo Evaluation of Dual-KIR T Cells in Xenograft NSG Mouse Treatment Models.

The anti-tumor activity of single and dual-KIR T cells was evaluated in well-established NOD-SCID-IL2Rγ_{c}^{-/-} (NSG) xenograft models of Burkitt's lymphoma using the Raji cell line. To model CD19 antigen loss, wild-type (CD19+CD20+) and CD19 KO (CD20+) Raji cells expressing CBG-GFP were mixed at a 3:1 ratio. Tumor progression was tracked in vivo by bioluminescent imaging (BLI) system (Ami HT, Spectral Instruments Imaging) and analyzed using Aura Image Software (Spectral Instruments Imaging). 5×10⁵ Raji-CBG-GFP (WT+CD19 KO) were intravenously (IV) injected into NSG mice housed in the CRADL facility under pathogen-free conditions in compliance with experimental protocols approved by the Institutional Animal Care and Use Committee. Five days after tumor injection, a 1×10⁶ suboptimal dose of CAR⁺ T cells (normalized to the lowest transduction efficiency) or untransduced (UTD) T cells (negative control) were IV injected into the mice after randomization. The photon flux of the ventral area of each mouse was analyzed by optical imaging twice per week following intraperitoneal injection of 150 mg/kg D-luciferin in groups of up to five mice at the same relative time point after luciferin injection. The tumor burden was evaluated using BLI. Retroorbital blood was taken and spun for plasma in order to measure cytokine levels. Whole blood collected was used to measure circulating peripheral T cells prior to spinning for plasma. Mice were then euthanized at pre-determined endpoints.

Cytokine detection via Meso Scale Discovery (MSD). Cytokine levels (human IFNT, TNFα,_and IL-2) were measured using a customized V-PLEX Proinflammatory Panel 1 Human Kit (MSD, K15049D). For in vitro studies, T cells and Raji WT, Raji CD19 KO, and Raji CD20 KO cells or K562 WT, K562 CD19 OE, and K562 CD20 OE cells were co-cultured at a 3:1 ratio of KIR or CAR+ cells to target ratio for 24 hours at 37 C. 50 μl media was collected, spun down to remove dead cells, and diluted 1:2 to detect TNFα and IL-2 and 1:1000 to detect IFNT. For in vivo studies, retro-orbital blood was collected from mice and spun down for plasma. Plasma was diluted 1:2 to detect TNFα, and IL-2, and 1:1000 to detect IFNT. For all studies, samples and standards were run in duplicate on the provided MSD plate and the protocol was carried out according to the provided kit instructions. The final plate readout was done on the MESO QuickPlex SQ 120 MM and data was analyzed using Discovery Workbench Software Version 4.0.

Analysis of T cell persistence in peripheral blood from tumor-bearing NSG mice. Total human T cells circulating in peripheral blood were quantified using CountBright™ Plus Ready Tubes (Invitrogen) pre-loaded with counting beads. Peripheral blood was collected retroorbitally from mice after tumor injection. Peripheral blood was added directly to CountBright™ Plus Ready Tubes and immediately lysed using RBC lysis buffer and blocked with TruStain FcX™ (anti-mouse CD16/32) (BioLegend). The cells were then stained with AlexaFluor700 anti-mouse CD45 (BioLegend) and PE anti-human CD3 antibody (BioLegend) and run on the Novocyte Penteon (Agilent). Flow cytometric analysis was carried out using FlowJo X 10.10.1 (Tree Star), and the absolute T cell number (cells/L) of was determined by comparing CD3 positive cellular events to bead events following manufacturer instructions.

Incucyte-based Cell Killing Assay. Tumor killing function KIR or CAR T cells was assessed by co-culture with K-562-WT, CD19-overexpressing, or CD20-overexpressing cells lines a 3:1 effector:target cell ratio (E:T ratio). The day of co-culture, 2×10⁴ target cells were plated in 100 μL per well of a flat-bottomed 96-well plate coated with poly-Ornithine (Sigma, Cat #P4957) to allow target cells to adhere. Frozen T cells were thawed the day prior to co-culture (DO) and allowed to rest overnight at 1-2×10⁶/mL density overnight in R10 media (RPMI1640+10% FBS) without cytokines. On D1, T cells were normalized to the same transduction efficiency using UTD cells and resuspended at a density of 3×10⁵/mL KIR or CAR+ cells. 100 μL or 33 μL of this mixture was added per well of target cells for a final E:T ratio of 3:1 Each condition was plated in triplicate. After a 30 minute equilibration at 37° C., plates were placed in an Incucyte SX1 Live-Cell analysis System (Sartorius). 3 images at 20× were acquired per well, and images were acquired every 3 hours for both brightfield and GFP filters for 72 hours. Target cell numbers per well over time were determined using Adherent Cell-by-Cell analysis and normalized to starting cell numbers.

Luciferase Killing Assay. Target cells were plated at 2×10⁴ cells/well, T cells at 3:1 and 1:1 CAR+ Effector:Target (E:T) ratios, and appropriate controls (full lysis and spontaneous death) in a 96-well plate and incubated for 18-24 hours in incubator at 37° C., 5% CO₂. For the full lysis control, 100 μL of target cells at 2×10⁴ cells/well were plated and 100 μL of 0.05% Tween solution added. For the spontaneous death control, plated 100 μL of target cells at 2×10⁴ cells/well+100 μL of untransduced (UTD) T cells at both the 3:1 and 1:1 E:T ratios. Bioluminescence of target cells was assayed according to manufacturer instructions of Promega's Luciferase Assay System (Promega, E1501) and read out on Varioskan Lux (Thermo Fisher Scientific) plate reader. Specific lysis was calculated according to the following equation:

$$\% Specific Lysis = 100 * [ \frac{Experimental RLU - Spontaneous Death RLU}{Full Lysis RLU - Spontaneous Death RLU} ]$$

Statistical Analysis. One-way ANOVA was used to compare groups. Multiple group comparisons were performed by a post hoc Tukey's HSD test. Significant differences were indicated as *p<0.05, **p<0.01, ***p<0.001, ****p<0.0001.

### Example 6—Elimination of Sequence Deletion Due to Lentiviral Template Switching

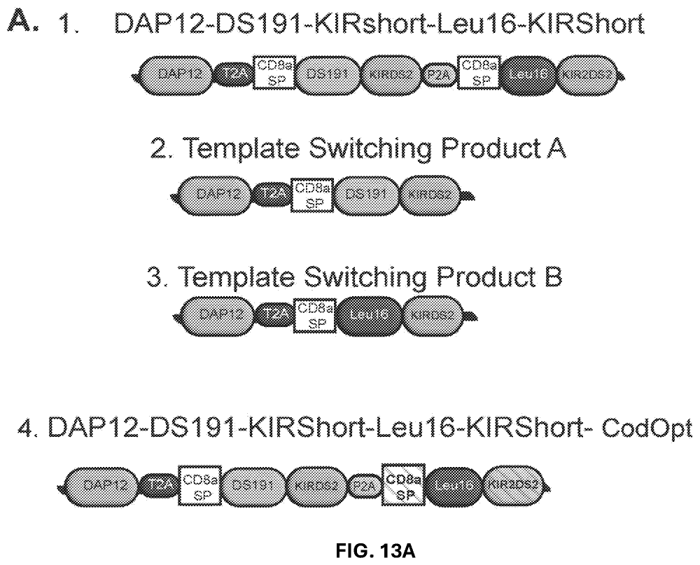

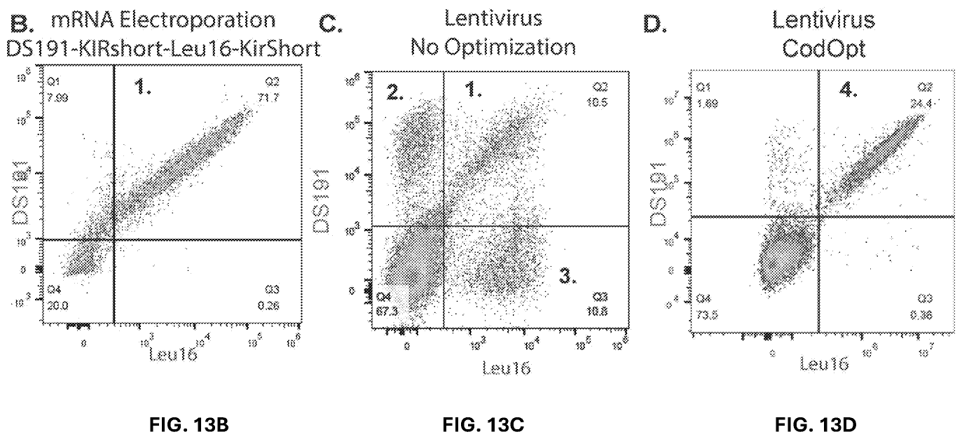

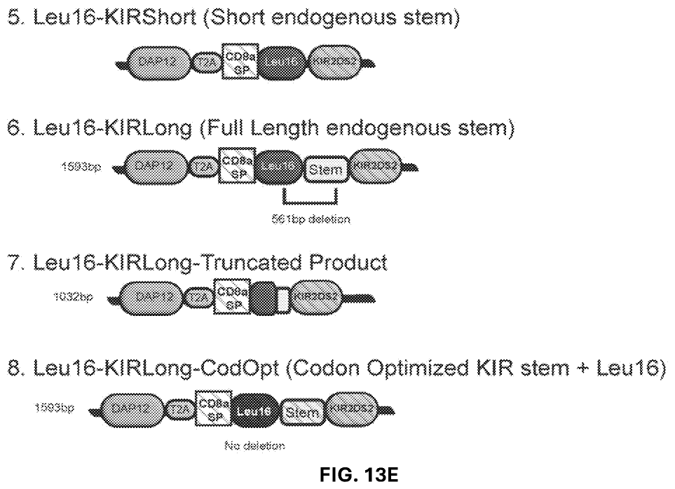

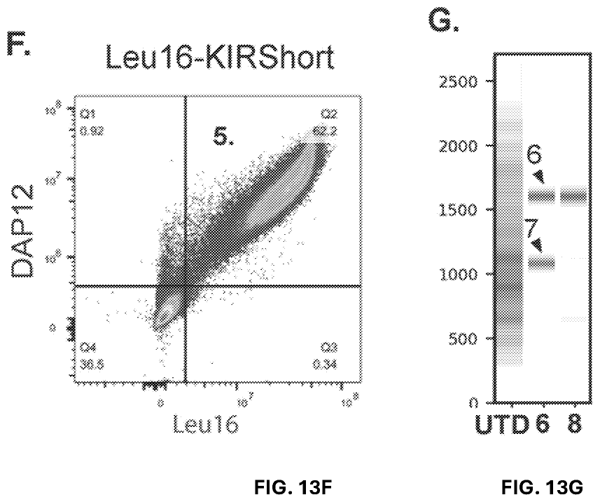

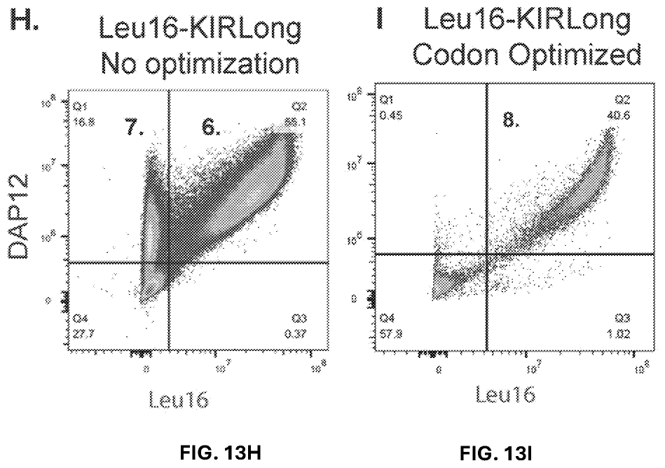

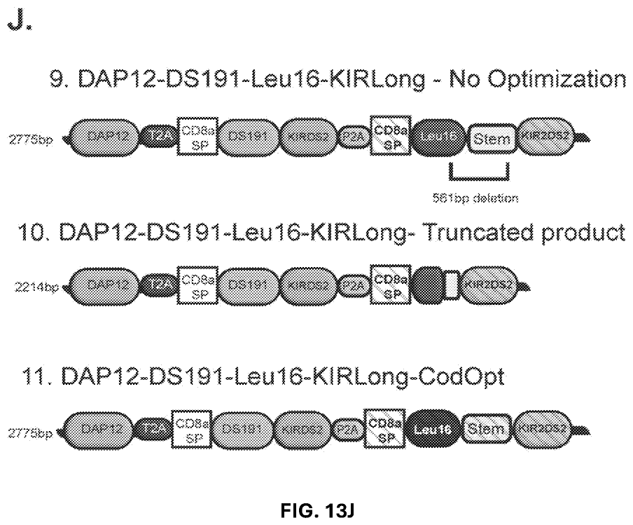

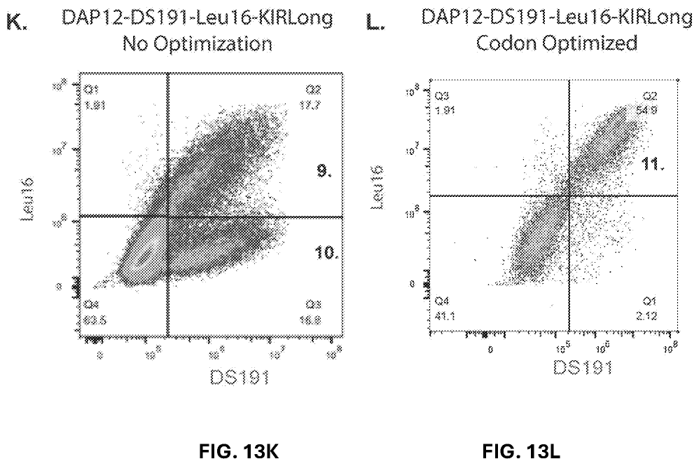

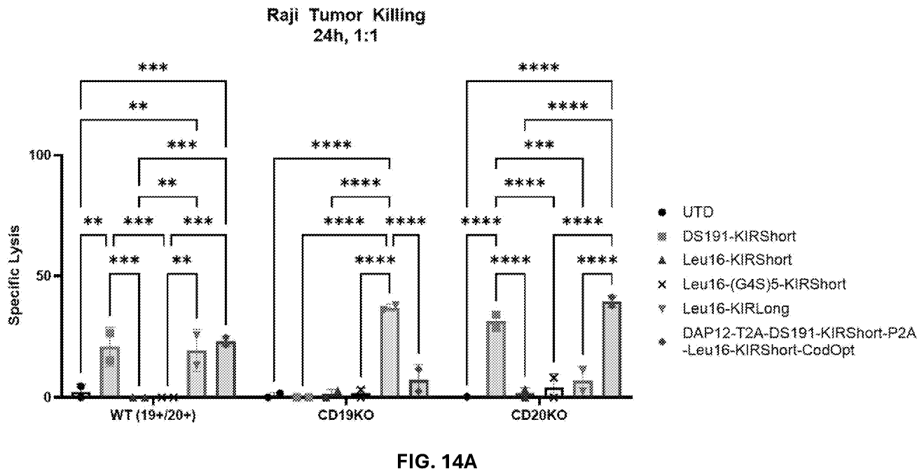

A number of optimizations were necessary to eliminate spurious recombinant proteins generated from genetic rearrangement within the transgene, as well as maximize activity of the Dual-KIR. The first optimizations were performed to eliminate lentiviral template switching. Initial Dual-KIR designs featured two copies each of the CD8α signal peptide sequence (69 bp; 23aa), as well as the KIR2DS2 stem/transmembrane sequence (225 bp; 75aa), with each duplicated region separated by ~1 kb. These designs unintentionally resulted in generation of single binder constructs (DAP12-DS191-KIRshort; DAP12-Leu16-KIRshort) during viral packaging due to template switching, in addition to the intended full length sequence (DAP12-T2A-DS191-KIRShort-Leu16-KIRShort (with (G4S)2 linker)). These spurious constructs were apparent by flow cytometry in T cells infected with lentivirus, but not those electroporated with the same sequence of mRNA (FIGS. 13A-13C). Template-switching is a well-documented phenomenon where the lentiviral reverse transcriptase “switches” from one RNA copy to the other at long regions of sequence homology during minus strand DNA synthesis (An, 2002, J Virol). Frequency of template switching increases with the length and degree of sequence homology, as well as distance between homologous regions, with a deletion frequency of ~50% for distances of 1 kb (Delviks, 1999, J Vir). This high degree of deletion frequency accounts for the roughly proportional distribution of T cells expressing each single or Dual KIR constructs (FIG. 13C). Codon optimization of one copy each of the CD8α signal peptide and KIR2DS2 sequences reduced homology <50% and eliminated generation of spurious single-KIR constructs while maintained amino acid sequence (FIG. 13D). All subsequent constructs utilized these non-homologous nucleotides sequences for each copy of the CD8α signal peptide and KIR2DS2 domains.

### Example 7—Elimination of Sequence Deletion Due to Cryptic Splicing Between Leu16 and the KIR2D2 Stem Domain in Leu16-KIRLong Constructs

Optimizations were performed next to improve the tumor lysis ability of the Leu16-KIR. Initial designs (DAP12-T2A-Leu16-KIRshort (with (G4S)2 linker), abbrv. Leu16-KIRshort) utilized the same short linker (G4S)2 between the ScFv and the KIR2DS2 stem domain used in the DS191-KIRshort single binder. However, the design showed poor tumor lysis activity against CD20 targets (FIG. 5). Therefore, the (G4S)2 linker was increased to (G4S)5 and an additional 24 amino acid sequence encoding the full-length endogenous KIR2D2 stem domain was added, generating the new longer linker sequence DAP12-T2A-Leu16-KIRLong (abbrv. Leu16-KIRLong) (FIG. 13E). However, while lentiviral delivery of DAP12-Leu16-KIRShort-CodOpt (abbrv. Leu16-KIRshort-CodOpt) construct showed all infected T cells expressing both Leu16 and DAP12, delivery of Leu16-KIRLong (with (G4S)2 linker) showed the presence of yet another spurious population that only expressed DAP12, but not Leu16 (FIG. 13H). PCR of genomic DNA from T cells identified the presence of two transgenes in Leu16-KIRLong infected cells (FIG. 13G). Long read sequencing showed the cause as a 561 bp deletion between the heavy chain of Leu16 and the full length KIR2DS2 stem domain. No sequence homology was identified between these two regions, eliminating the possibility of template switching as the cause. This time, cryptic splicing was identified as the culprit, resulting in a spurious “alternative isoform” of the Leu16-KIRLong (with (G4S)5 linker). The causative splice donor/acceptor nucleotide regions were able to be identified within the Leu16 heavy chain region and KIR2DS stem sequences (Wang and Marin, 2006, Gene) the nucleotides were optimized to eliminate these sites while maintaining amino acid sequence. The final design, DAP12-T2A-Leu16-KIRLong-Opt (with (G4S)5 linker) (abbrv. Leu16-KIRLong-Opt) showed no apparent splicing by PCR (FIG. 13G), and Leu16-KIRLong-Opt T cells exclusively showed expression of both DAP12 and Leu16 by flow cytometry. This optimization strategy showed the same patterns expression when incorporated into the dual-KIR format (FIG. 13J-L). Initial unoptimized DAP12-T2A-DS191-Leu16-KIRLong (with (G4S)5 linker) T cells also showed the same 561 deletion as the Leu16-KIRLong (with (G4S)5 linker) construct, and expression of DAP12-DS191 single binder expressing constructs (FIG. 13K). Once again, codon optimization of the nucleotides responsible for cryptic splicing restored expression of both DS191 and Leu16 on all DAP12-T2A-DS191-Leu16-KIRLong-CodOpt T cells (FIG. 13L).

### Example 8—Increasing the Linker Length Between Leu16 and the KIR2D Transmembrane Domain Improves Anti-CD20 Tumor Lysis In Vitro and In Vivo

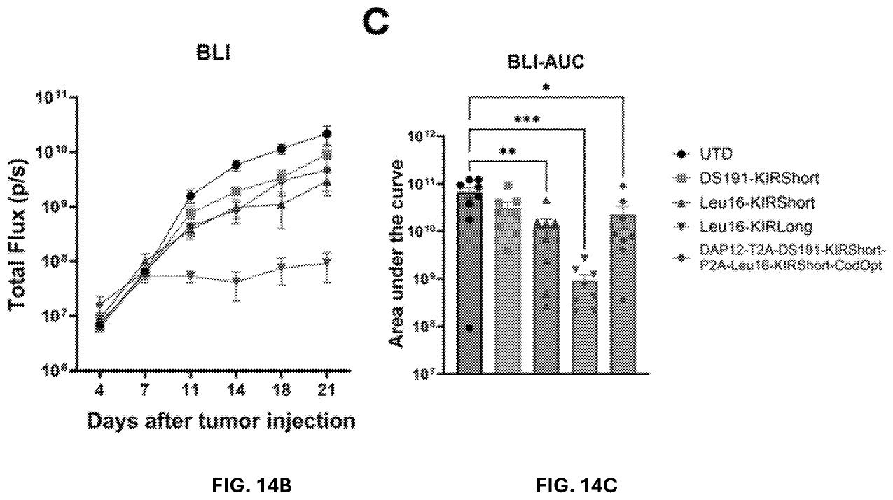

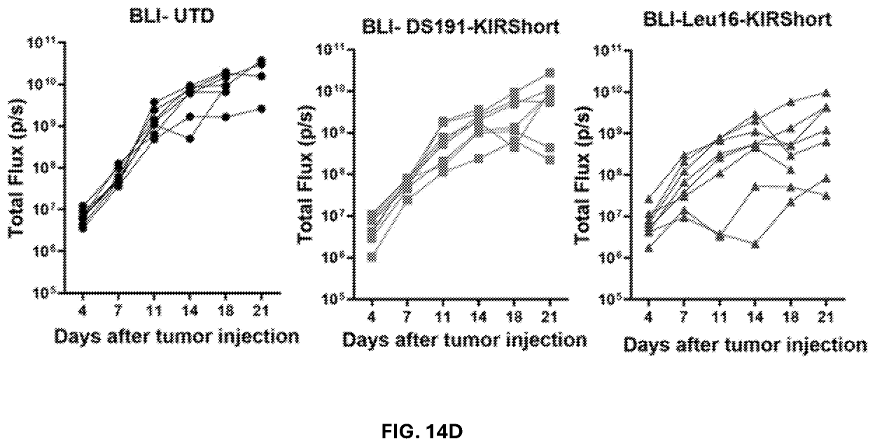

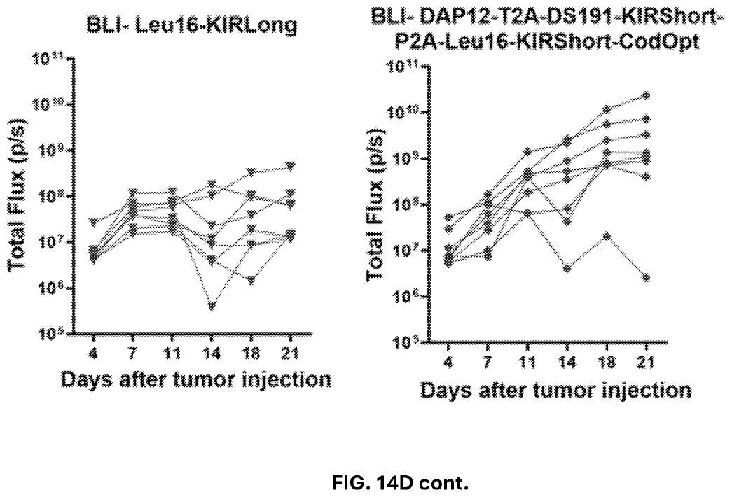

Raji WT (CD19+ and CD20+), Raji CD19 KO (CD20+) and Raji CD20 KO (CD19+) cells were plated at 2×10⁴ cells/well and CAR+ T cells were plated at a 1:1 CAR+ Effector:Target (E:T) ratio in a 96-well plate and incubated for 18-24 hours in an incubator at 37° C., 5% CO₂. Bioluminescence of target cells was assayed according to manufacturer instructions of Promega's Luciferase Assay System (Promega, E1501) and read out on Varioskan Lux (Thermo Fisher Scientific) plate reader. For this assay, three Leu16-KIR T cell groups were compared: the original Leu16-KIRShort (n=2 technical replicates), a longer Leu16-(G4S)5-KIRShort with additional G4S linkers (n=2 technical replicates), and a longer Leu16-KIRLong (with (G4S)5 linker) with added full length KIR2D stem domain (n=2 technical replicates). Negative control untransduced (UTD) T cells (n=2 technical replicates), DS191-KIRShort T cells (n=2 technical replicates), and DAP12-T2A-DS191-KIRShort-P2A-Leu16-KIRShort-CodOpt T cells (n=2 technical replicates) were also included. Leu16-KIRLong demonstrated the greatest tumor killing ability of all Leu16-KIR groups, while Leu16-KIRShort and Leu16-(G4S)5-KIRShort failed to demonstrate any tumor killing ability, against both Raji WT and Raji CD19 KO cells (FIG. 14A). Leu16-KIRLong also demonstrated improved tumor killing compared to DAP12-T2A-DS191-KIRShort-P2A-Leu16-KIRShort-CodOpt against Raji CD19 KO cells, highlighting the need to improve Leu16-KIR activity in order to improve Dual KIR functionality (FIG. 14A). Raji CD20 KO cells were included as a control, as no Leu16-KIR killing should occur against CD20 KO cells, and that is what we observed.

To evaluate the in vivo anti-tumor efficacy of Leu16-KIRLong (with (G4S)₅ linker) T cells, compared to untransduced (UTD), Leu16-KIRShort, DS191-KIRShort, and DAP12-T2A-DS191-KIRShort-P2A-Leu16-KIRShort-CodOpt, immunocompromised NSG mice were IV injected with 5×10⁵ luciferase-expressing mixed Raji-CBG GFP tumor cells (75% WT Raji, 25% CD19 KO Raji, to model antigen escape). After five days, animals were randomized, and then injected IV with a 1×10⁶ CAR⁺ dose of Leu16-KIRLong (n=8), Leu16-KIRShort (n=8), DS191-KIRShort (n=8), and DAP12-T2A-DS191-KIRShort-P2A-Leu16-KIRShort-CodOpt (n=8) T cells, plus a negative control untransduced (UTD, n=8) group, and all were normalized to the lowest transduction efficiency. Bioluminescence imaging (BLI) of mixed Raji tumor growth was performed using an Ami HT Spectral Instruments Imager twice weekly (FIGS. 14B,14C, and 14D). The negative control UTD, Leu16-KIRShort, DS191-KIRShort, and DAP12-T2A-DS191-KIRShort-P2A-Leu16-KIRShortCodOpt T cell-treated groups exhibited rapid tumor progression and tumor burden measured by BLI that continuously increased up to Day 21. In contrast, Leu16-KIRLong—was the only T cell treatment that improved tumor control and reduced tumor burden by BLI (FIGS. 14B, 14C, and 14D). Overall tumor control measured by area under the curve (AUC) showed that all Leu16-containing T cell groups were able to demonstrate some degree of tumor control compared to UTD negative control mice, however, Leu16-KIRLong (with (G4S)5 linker) T cells clearly showed the most robust tumor control (FIG. 14C). Overall, the results of this study reveal that Leu16-KIRLong (with (G4S)5 linker) has improved tumor killing over Leu16-KIRShort both in vitro and in vivo. Results in vivo revealed a 13-fold reduction in tumor burden by Leu16-KIRLong (with (G4S)5 linker) compared to Leu16-KIRShort. And, Leu16-KIR's functionality is essential to Dual KIR tumor killing, as the DAP12-T2A-DS191-KIRShort-P2A-Leu16-KIRShort-CodOpt T cell in vivo tumor killing ability was only as good as that of the Leu16-KIRShort T cell killing ability. We aim to improve Dual KIR functionality by incorporating Leu16-KIRLong into the Dual KIR construct.

### Example 9—Increasing the Linker Length Between Leu16 and the KIR2D Transmembrane Domain Improves Anti-CD20 Tumor Lysis in the Dual Binder In Vitro

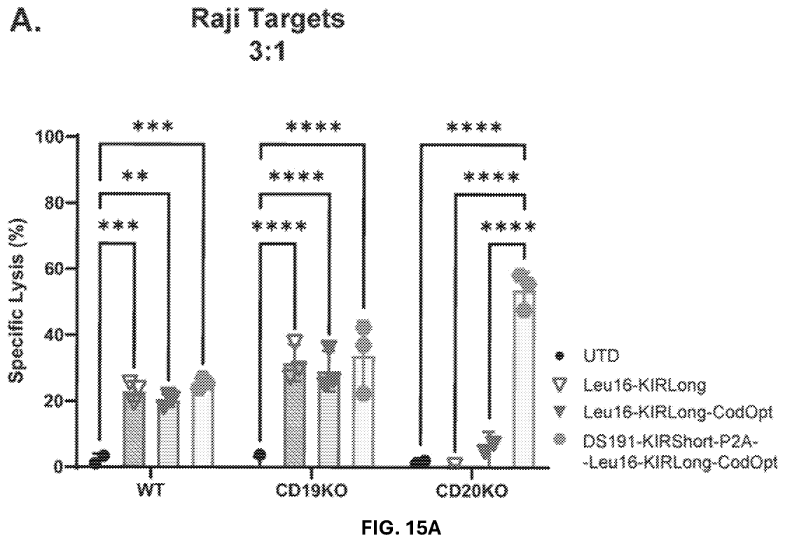

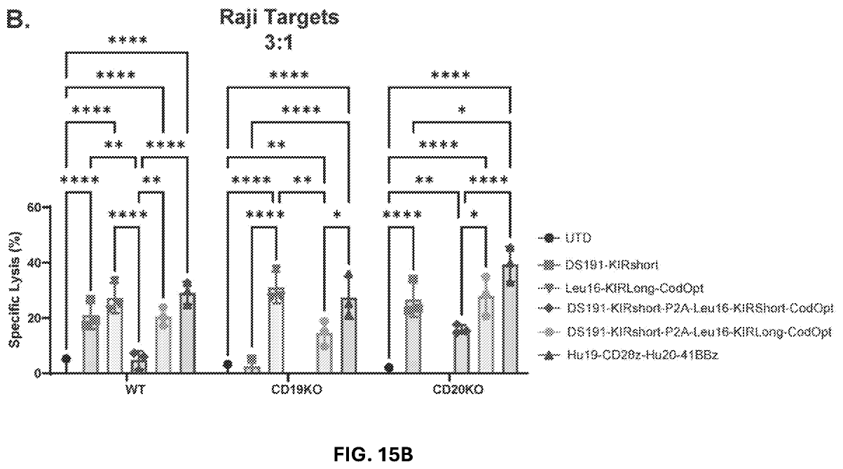

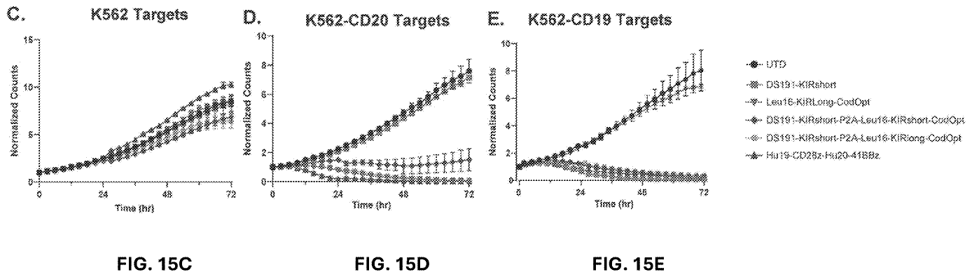

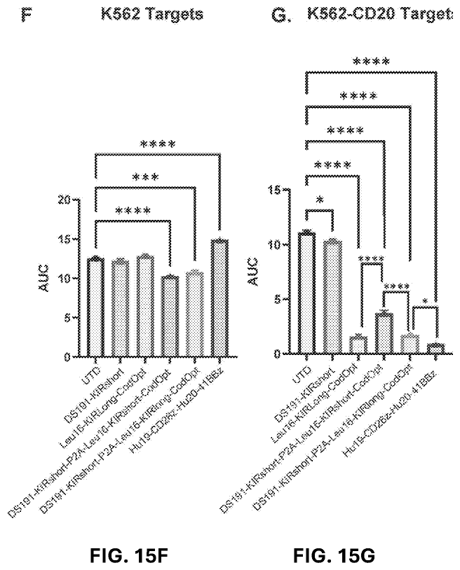

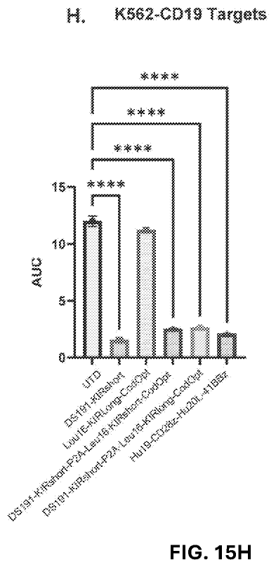

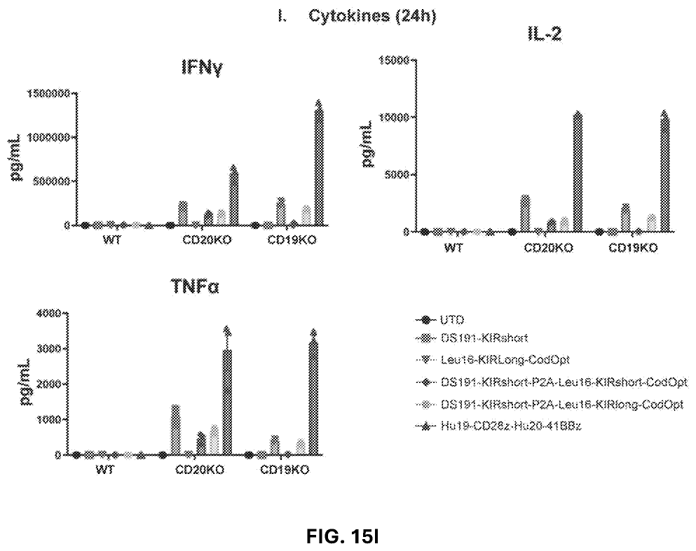

The functionality of the Leu16-KIRLong-CodOpt (with (G4S)5 linker) design was next evaluated in the Dual-KIR format in vitro. T cells were incubated for 24 hours with WT (CD19+/CD20+), CD19KO or CD20KO Raji target cells and cell lysis evaluated by BLI. As a single binder, the Leu16-KIRLong-CodOpt (with (G4S)5 linker) showed equivalent tumor lysis to the Leu16-KIRLong (with (G4S)5 linker) (no optimization), as expected (FIG. 15A) against all CD20+ target types, with no off-target lysis of CD20KO target cells. The dual-KIR, DAP12-T2A-DS191-KIRShort-P2A-Leu16-KIRLong-CodOpt also showed equivalent performance against CD20+ vs the single binder Leu16-KIRLong-CodOpt alone, in addition to tumor killing against CD20KO (CD19 only) targets. Activity of the DAP12-T2A-DS191-KIRShort-P2A-Leu16-KIRLong-CodOpt design was next compared to the prior DAP12-T2A-DS191-KIRShort-P2A-Leu16-KIRShort-CodOpt, the single binders DS191-KIR short and Leu17-KIRLong-CodOpt, as well as a bicistronic, traditional CAR T construct described by Lam et al (2023, Mol Ther Oncolytics). This bicistronic construct, shown here as huCD19-28z-huCD20-41BBz, utilizes humanized CD19 and CD20 binders on two separate single-chain CD3z-based CAR constructs that incorporate as CD28 or 41BB co-stimulatory domain (respectively). As expected, DAP12-T2A-DS191-KIRShort-P2A-Leu16-KIRLong-CodOpt significantly outperformed DAP12-T2A-DS191-KIRShort-P2A-Leu16-KIRShort-CodOpt, with the latter showing no tumor lysis against CD19KO Raji targets. DAP12-T2A-DS191-KIRShort-P2A-Leu16-KIRLong-CodOpt showed similar tumor lysis vs the huCD19-28z-huCD20-41BBz construct (FIG. 15B). Similar results were seen when evaluating tumor lysis of dual and single KIRs against K562 WT (CD19−/CD20−), K562-CD20 overexpressing (K562-CD200E) or CD19 overexpressing (K562-CD19OE) target cells (FIG. 15C-15H). Tumor killing was evaluated by Incucyte assay over 72 hr. Minimal reactivity against K562 WT target cells was observed for all dual and single-KIRs, as well as the huCD19-28z-huCD20-41BBz bicistronic CARs. As with the Raji targets, the DAP12-T2A-DS191-KIRShort-P2A-Leu16-KIRLong-CodOpt dual-KIR was superior to the DAP12-T2A-DS191-KIRShort-P2A-Leu16-KIRShort-CodOpt design against CD20 targets (FIG. 15D,15G) and demonstrated equivalent tumor to each single-KIR design (DS191-KIRshort; Leu16-KIRLong-CodOpt) against relevant targets. Once again, despite similar tumor lysis to the huCD19-28z-huCD20-41BBz bicistronic CAR, the DAP12-T2A-DS191-KIRShort-P2A-Leu16-KIRLong-CodOpt design showed 10-fold lower cytokine release (FIG. 15I), suggesting an improved safety profile of the dual-KIR format over a dual CD3zeta-based CAR.

### Example 10—DAP12-T2A-DS191-KIRShort-P2A-Leu16-KIRLong-CodOpt Shows Superior Performance Compared to DAP12-T2A-DS191-KIRShort-P2A-Leu16-KIRShort-CodOpt, and Reduced Cytokines Compared to Hu19-CD28z-huCD20-41BBz In Vivo

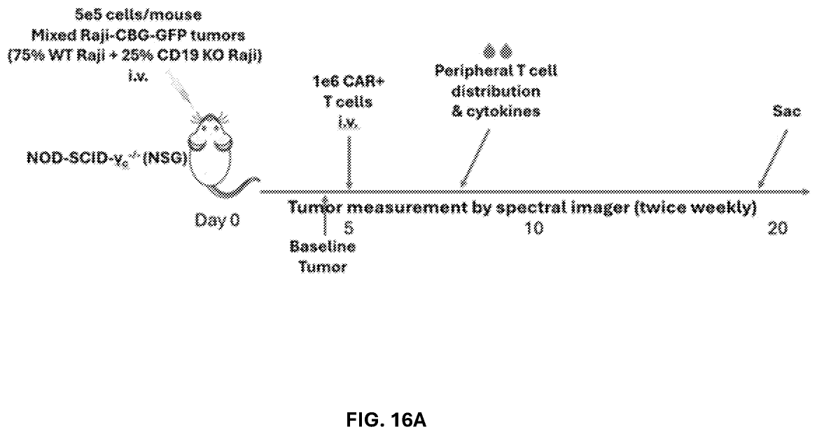

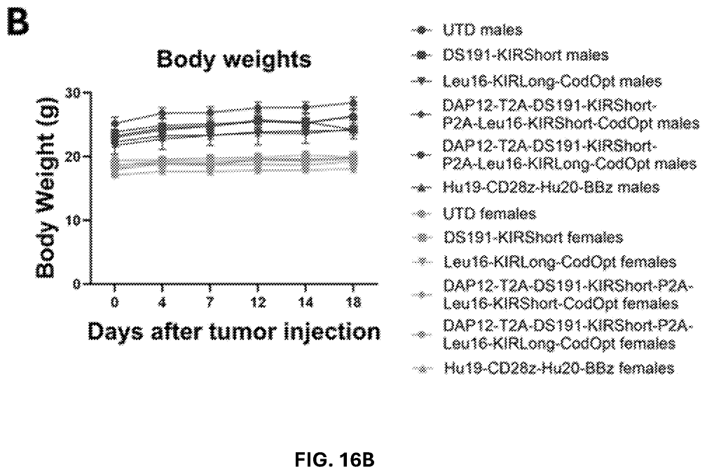

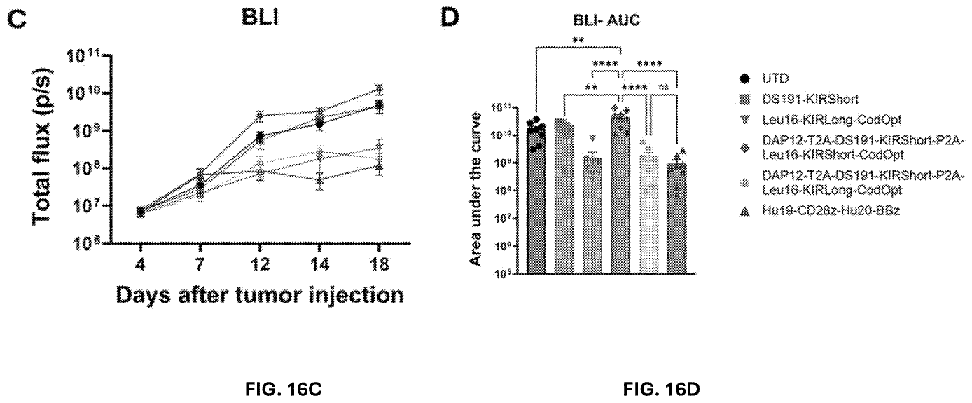

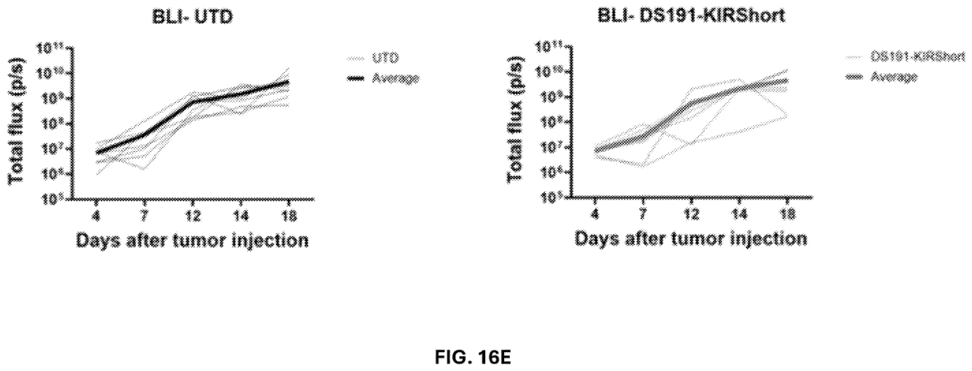

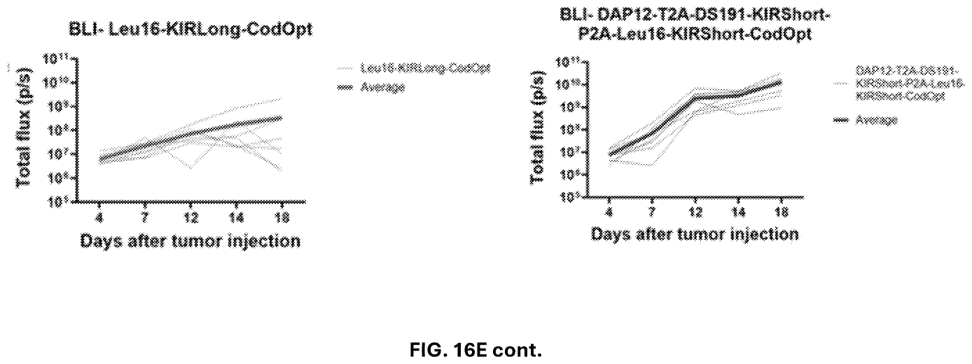

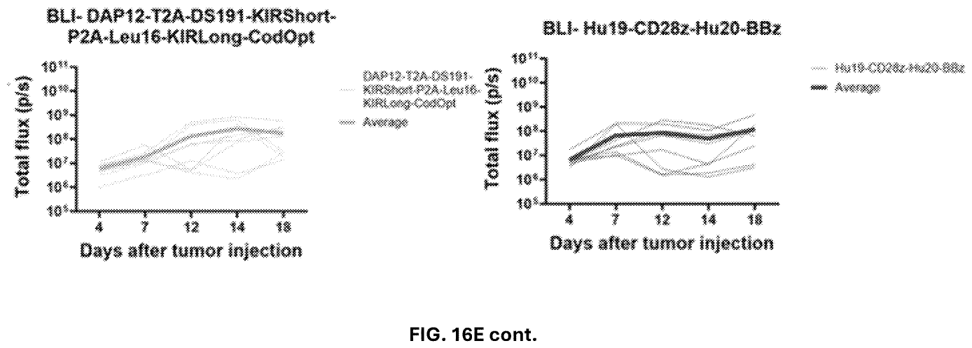

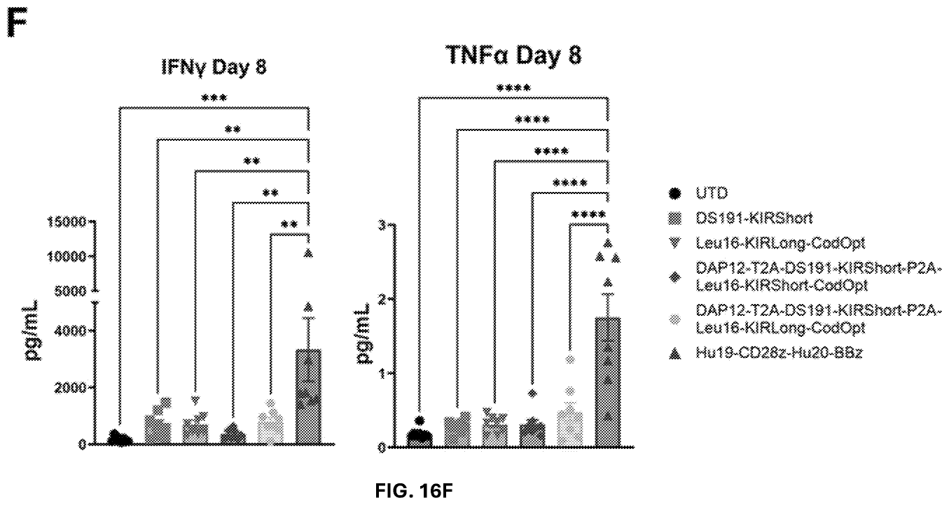

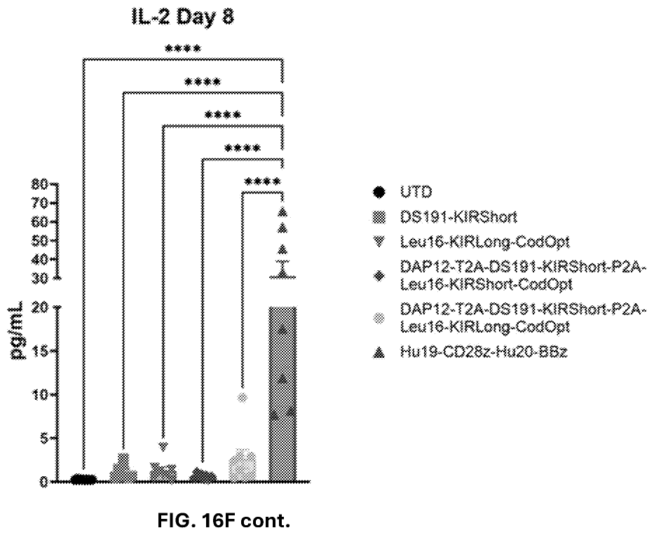

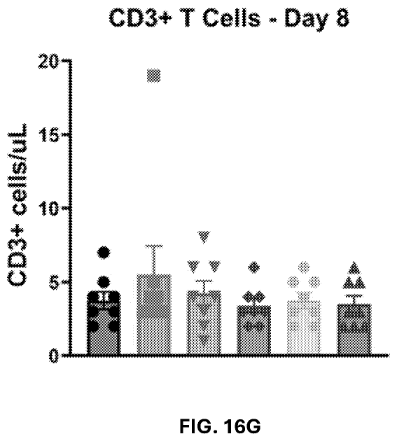

To evaluate the in vivo anti-tumor efficacy of the fully optimized DAP12-T2A-DS191-KIRShort-P2A-Leu16-KIRLong-CodOpt compared to both single binders (DS191-KIRShort and Leu16-KIRLong-CodOpt), DAP12-T2A-DS191-KIRShort-P2A-Leu16-KIRShort-CodOpt, and Hu19-CD28z-huCD20-41BBz (a CD3-based, single chain bicistronic CAR targeting both CD19 and CD20), the same in vivo study set-up was used as in FIG. 14. Immunocompromised NSG mice were IV injected with 5×10⁵ luciferase-expressing mixed Raji-CBG GFP tumor cells (75% WT Raji, 25% CD19 KO Raji, to model antigen escape). After five days, animals were randomized, and then injected IV with a 1×10⁶ CAR⁺ dose of DS191-KIRShort (n=8), Leu16-KIRLong-CodOpt (n=8), DAP12-T2A-DS191-KIRShort-P2A-Leu16-KIRLongCodOpt (n=8), DAP12-T2A-DS191-KIRShort-P2A-Leu16-KIRShortCodOpt (n=8), and Hu19-CD28z-huCD20-41BBz (n=8) T cells, plus a negative control untransduced (UTD, n=8) group, and all were normalized to the lowest transduction efficiency (FIG. 16A). Body weights were taken twice weekly throughout the experiment (FIG. 16B). Bioluminescence imaging (BLI) of mixed Raji tumor growth was performed using an Ami HT Spectral Instruments Imager twice weekly (FIGS. 16C and 16D). The negative control UTD, DS191-KIRShort, and DAP12-T2A-DS191-KIRShort-P2A-Leu16-KIRShortCodOpt T cell-treated groups exhibited rapid tumor progression and tumor burden measured by BLI that continuously increased up to Day 18. In contrast, Leu16-KIRLong-CodOpt, DAP12-T2A-DS191-KIRShort-P2A-Leu16-KIRLong-CodOpt, and Hu19-CD28z-huCD20-41BBz-treated mice showed improved tumor control and reduced tumor burden by BLI compared to UTD, DS191-KIRShort, and DAP12-T2A-DS191-KIRShort-P2A-Leu16-KIRShortCodOpt T cell-treated groups (FIGS. 16C, 16D, and 16E). Overall tumor control measured by area under the curve (AUC) was comparable between Leu16-KIRLong-CodOpt, DAP12-T2A-DS191-KIRShort-P2A-Leu16-KIRLong-CodOpt, and Hu19-CD28z-huCD20-41BBz. Most notably, DAP12-T2A-DS191-KIRShort-P2A-Leu16-KIRLong-CodOpt and Leu16-KIRLong-CodOpt demonstrated significant reduction (25-fold and 28-fold, respectively) in overall tumor burden compared to DAP12-T2A-DS191-KIRShort-P2A-Leu16-KIRShort demonstrating that the improved Leu16-KIRLong-CodOpt is essential to Dual KIR functionality.

Peripheral cytokine levels were also evaluated at Day 8 after tumor injection (FIG. 16F). Retroorbital blood was collected at Day 8 and whole blood was collected for evaluation of T cell persistence, and the remaining blood was spun down for plasma. The plasma was then run in duplicate on Meso Scale Discovery (MSD) plates to measure IFNγ, TNFα, and IL-2. Day 8 was an early timepoint, only 3 days post T cell injection, so IFNγ, TNFα, and IL-2 concentrations were comparably low in mice treated with UTD (n=8), DS191-KIRShort (n=8), Leu16-KIRLong-CodOpt (n=8), DAP12-T2A-DS191-KIRShort-P2A-Leu16-KIRLongCodOpt (n=8), and DAP12-T2A-DS191-KIRShort-P2A-Leu16-KIRShortCodOpt (n=8) T cells. However, mice treated with Hu19-CD28z-huCD20-41BBz (n=8) T cells secreted significantly more of all three cytokines IFNγ, TNFα, and IL-2 compared to all other groups. Importantly, while tumor control was comparable between Hu19-CD28z-huCD20-41BBz and DAP12-T2A-DS191-KIRShort-P2A-Leu16-KIRLongCodOpt, Hu19-CD28z-huCD20-41BBz secreted 4.2-fold more IFNγ (3325.7 pg/mL vs. 791.1 pg/mL), 3.7-fold more TNFα (1.75 g/mL vs. 0.47 pg/mL), and 11.8-fold more IL-2 (30.8 g/mL vs. 2.6 pg/mL) than DAP12-T2A-DS191-KIRShort-P2A-Leu16-KIRLong-CodOpt, suggesting that DAP12-T2A-DS191-KIRShort-P2A-Leu16-KIRLong-CodOpt is able to control tumor burden with reduced levels of proinflammatory cytokines.

To evaluate T cell persistence across groups, we measured the amount of circulating peripheral T cells in the blood of tumor-bearing mice treated with UTD (n=8), DS191-KIRShort (n=8), Leu16-KIRLong-CodOpt (n=8), DAP12-T2A-DS191-KIRShort-P2A-Leu16-KIRLongCodOpt (n=8), DAP12-T2A-DS191-KIRShort-P2A-Leu16-KIRShortCodOpt (n=8), and Hu19-CD28z-huCD20-41BBz (n=8) T cells at Day 8 by collecting whole blood retroorbitally. Peripheral blood was added to CountBright™ Plus Ready Tubes (Invitrogen) pre-loaded with counting beads then lysed using RBC lysis buffer and blocked with TruStain FcX™ (anti-mouse CD16/32) (BioLegend). The cells were then stained with AlexaFluor700 anti-mouse CD45 (BioLegend) and PE anti-human CD3 antibody (BioLegend) and run on the Novocyte Penteon (Agilent). Data were analyzed with FlowJo version X (TreeStar). All mice had an average of 3-6 T cells/μL of peripheral blood, as this was an early timepoint at 3 days post T cell injection. There was no difference in the number of circulating T cells across all groups (FIG. 16G). The data suggests comparable peripheral T cell persistence across all groups at Day 8.

The references cited throughout this application are incorporated for all purposes apparent herein and in the references themselves in their entireties. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A composition comprising one or more recombinant nucleic acid molecules:
a. a first nucleic acid sequence encoding a first fusion protein comprising:
(i) an antigen-binding domain comprising an anti-CD19 amino acid sequence;
(ii) a transmembrane domain comprising a KIR polypeptide; and
(iii) a cytoplasmic domain comprising a KIR polypeptide; and
b. a second nucleic acid sequence encoding a second fusion protein comprising:
(iii) an antigen-binding domain comprising an anti-CD20 amino acid sequence;
(iv) a transmembrane domain comprising a KIR polypeptide; and
(v) a cytoplasmic domain comprising a KIR polypeptide.

2. The composition of claim 1, further comprising a third nucleic acid sequence encoding a DAP12 polypeptide.

3. (canceled)

4. The composition of claim 1, wherein the first and second nucleic acid sequences each comprise a nucleic acid sequence encoding a KIR2DS2 domain.

5-9. (canceled)

10. The composition of claim 1, wherein the antigen-binding domain comprising an anti-CD19 amino acid sequence comprises an anti-CD19 antibody or an antigen-binding fragment thereof.

11. (canceled)

12. (canceled)

13. The composition of claim 1, wherein the antigen-binding domain comprising an anti-CD19 amino acid sequence comprises
a heavy chain variable region (VH) that comprises a first heavy chain complementarity determining region (HCDR1) having the sequence GFTLSNYG (SEQ ID NO: 163), a second heavy chain complementarity determining region (HCDR2) having the sequence IRSGGNTY (SEQ ID NO: 164), and a third heavy chain complementarity determining region (HCDR3) having the sequence SQWGDWGHFEY (SEQ ID NO: 123); and
a light chain variable region (VL) that comprises a first light chain complementarity determining region (LCDR1) having the sequence RSNIGANY (SEQ ID NO: 124), a second light chain complementarity determining region (LCDR2) having the sequence GYN, and a third light chain complementarity determining region (LCDR3) having the sequence STWDSSLNAYV (SEQ ID NO: 165).

14. (canceled)

15. The composition of claim 1, wherein the antigen-binding domain comprising an anti-CD19 amino acid sequence comprises a VH comprising a sequence having at least 90%, 95%, 96%, 97%, 98%, 99%, or 100% sequence identity to the sequence EVPLVESGGDLVKPGGSLRLSCVASGFTLSNYGMSWVRQSPGKGLQWVAAIRSGGNTYYA DAVKGRFTISRDNAKNTLYLQMNSLRAEDTAVYYCASQWGDWGHFEYWGQGTLVTVSS (SEQ ID NO: 331) and a VL comprising a sequence having at least 90%, 95%, 96%, 97%, 98%, 99%, or 100% sequence identity to the sequence QSALTQTASVSGSLGQKVTISCTGSRSNIGANYVGWYQQVPGIGPRTVIFGYNYRPSGVPDR FSSSKSGNSATLTISGLQAEDEAEYYCSTWDSSLNAYVFGSGTQLTVL (SEQ ID NO: 288).

16. (canceled)

17. (canceled)

18. The composition of claim 1, wherein the antigen-binding domain comprising an anti-CD19 amino acid sequence comprises a sequence having at least 90%, 95%, 96%, 97%, 98%, 99%, or 100% sequence identity to the sequence QSALTQTASVSGSLGQKVTISCTGSRSNIGANYVGWYQQVPGIGPRTVIFGYNYRPSGVPDR FSSSKSGNSATLTISGLQAEDEAEYYCSTWDSSLNAYVFGSGTQLTVLGGGSSRSSSSGGGG SGGGGEVPLVESGGDLVKPGGSLRLSCVASGFTLSNYGMSWVRQSPGKGLQWVAAIRSGG NTYYADAVKGRFTISRDNAKNTLYLQMNSLRAEDTAVYYCASQWGDWGHFEYWGQGTL VTVSS (SEQ ID NO: 61).

19. (canceled)

20. The composition of claim 1, wherein the antigen-binding domain comprising an anti-CD20 amino acid sequence comprises an anti-CD20 antibody or an antigen-binding fragment thereof.

21. (canceled)

22. (canceled)

23. The composition of claim 1, wherein the antigen-binding domain comprising an anti-CD20 amino acid sequence comprises
a heavy chain variable region (VH) that comprises a first heavy chain complementarity determining region (HCDR1) having the sequence SYNMH (SEQ ID NO: 803), a second heavy chain complementarity determining region (HCDR2) having the sequence AIYPGNGDTSYNQKFKG (SEQ ID NO: 804), and a third heavy chain complementarity determining region (HCDR3) having the sequence SNYYGSSYWFFDV (SEQ ID NO: 805); and
a light chain variable region (VL) that comprises a first light chain complementarity determining region (LCDR1) having the sequence RASSSVNYMD (SEQ ID NO: 800), a second light chain complementarity determining region (LCDR2) having the sequence ATSNLAS (SEQ ID NO: 801), and a third light chain complementarity determining region (LCDR3) having the sequence QQWSFNPPT (SEQ ID NO: 802).

24. (canceled)

25. The composition of claim 1, wherein the antigen-binding domain comprising an anti-CD20 amino acid sequence comprises a VH comprising a sequence having at least 90%, 95%, 96%, 97%, 98%, 99%, or 100% sequence identity to the sequence EVQLQQSGAELVKPGASVKMSCKASGYTFTSYNMHWVKQTPGQGLEWIGAIYPGNGDTS YNQKFKGKATLTADKSSSTAYMQLSSLTSEDSADYYCARSNYYGSSYWFFDVWGAGTTV TVSS (SEQ ID NO: 585) and a VL comprising a sequence having at least 90%, 95%, 96%, 97%, 98%, 99%, or 100% sequence identity to the sequence DIVLTQSPAILSASPGEKVTMTCRASSSVNYMDWYQKKPGSSPKPWIYATSNLASGVPARF SGSGSGTSYSLTISRVEAEDAATYYCQQWSFNPPTFGGGTKLEIK (SEQ ID NO: 537).

26. (canceled)

27. (canceled)

28. The composition of claim 1, wherein the antigen-binding domain comprising an anti-CD20 amino acid sequence comprises a sequence having at least 90%, 95%, 96%, 97%, 98%, 99%, or 100% sequence identity to the sequence EVQLQQSGAELVKPGASVKMSCKASGYTFTSYNMHWVKQTPGQGLEWIGAIYPGNGDTS YNQKFKGKATLTADKSSSTAYMQLSSLTSEDSADYYCARSNYYGSSYWFFDVWGAGTTV TVSSGSTSGGGSGGGSGGGSSDIVLTQSPAILSASPGEKVTMTCRASSSVNYMDWYQKKPG SSPKPWIYATSNLASGVPARFSGSGSGTSYSLTISRVEAEDAATYYCQQWSFNPPTFGGGTK LEIK (SEQ ID NO: 819).

29. (canceled)

30. (canceled)

31. The composition of claim 1, wherein each of the first fusion protein and the second fusion protein comprise a DAP12 association sequence, the DAP12 association sequence of the first fusion protein comprises the amino acid sequence of SEQ ID NOS: 753 or a functional variant thereof that comprises at least about 75% sequence identity SEQ ID NOS: 753, and the DAP12 association sequence of the second fusion protein comprises the amino acid sequence of SEQ ID NO: 753 or a functional variant thereof that comprises at least about 75% sequence identity to a respective one of SEQ ID NOS: 753.

32-37. (canceled)

38. The composition of claim 1, wherein the first nucleic acid sequence and the second nucleic sequence are encoded on the same nucleic acid molecule.

39-41. (canceled)

42. The composition of claim 1, wherein the recombinant nucleic acid molecule comprises, from 5′ to 3′, a sequence encoding DAP12; a sequence encoding a T2A ribosome skipping peptide; a sequence encoding the first fusion protein; a sequence encoding a P2A ribosome skipping peptide; and a sequence encoding the second fusion protein.

43. The composition of claim 1, wherein the recombinant nucleic acid molecule comprises a sequence encoding the amino acid sequence of SEQ ID NO: 720.

44. A composition comprising one or more polypeptides encoded by the one or more recombinant nucleic acid molecules of the composition of claim 1.

45. A cell comprising
(a) one or more recombinant nucleic acid molecules of the composition of claim 1 or
(b) the one or more polypeptides encoded by (a).

46-49. (canceled)

50. A population of cells comprising
(a) one or more recombinant nucleic acid molecules of the composition of claim 1 or
(b) one or more polypeptides encoded by (a).

51. A pharmaceutical composition comprising
(a) one or more recombinant nucleic acid molecules of the composition of claim 1 and a pharmaceutically acceptable carrier;
(b) one or more polypeptides encoded by the recombinant nucleic acid molecules of (a) and a pharmaceutically acceptable carrier;
(c) a cell comprising (a) or (b) and a pharmaceutically acceptable carrier: or
(d) a population of cells comprising (a) or (b) and a pharmaceutically acceptable carrier.

52-54. (canceled)

55. A method of treatment of a B cell-mediated disorder in a subject in need thereof comprising administering to the subject a pharmaceutical composition of claim 51.

56-59. (canceled)

60. A recombinant nucleic acid molecule comprising from 5′ to 3′:
a. a nucleic acid sequence encoding a DAP12 polypeptide, including an extracellular, transmembrane, and cytoplasmic domain;
b. a nucleic acid sequence encoding a T2A ribosomal skip peptide;
c. a nucleic acid sequence encoding an anti-CD19 antigen binding domain operably linked to a KIR2DS2-derived transmembrane, short extracellular domain, and intracellular domain
d. a nucleic acid sequence encoding a P2A ribosomal skip peptide; and
e. a nucleic acid sequence encoding an anti-CD20 antigen binding domain operably linked to a KIR2DS2-derived transmembrane, long extracellular domain, and intracellular domain.

61. (canceled)


SEQUENCE LISTING (845 sequences)

SEQ ID NO: 1 - DNA, 750 bp, Synthetic construct
  source 1..750: mol_type = other DNA
caggctgtgctgaatcagccggcctctgtgtctgcagccctggggcagagggtcaccatctcctgtaacaccaacatcggcagtccttatgatgtacaatggtaccagcagctcccaggaaagtcccctaaaactatcatttatggtaatagcaatcgaccctcgggggtcccggttcgattctctggctccaagtcaggcagcacagccaccctgaccatcgctgggatccaggctgaggatgaggctgattattactgccagtcctatgatgacagcctcgatggtcgtgttgtgttcggcggagggacccaactgaccgtcctcggtggtggttcctctagatcttcctcctctggtggcggtggctcgggcggtggtggggaggtgcagctggtggagtctgggggagacctggtgaagcctggggggtccttgagactctcctgtgtggcctctggattcaccttcagtaaccacgaaatgtactgggtccgccaggctccagggaaagggctggagtgggtcgcaaggatttatgatagtggaagtagaacaaggtatgcagacgctgtgaagggccgattcaccatctccagagacaacgccaagaacacgctgtatcttcagatgaacagcctgacagccgaggacacggccgtttatttctgtgcgggggggaattctcgtggttggaccgagtttggtatggactactggggcccgggaaccctggtcaccgtctcctca

SEQ ID NO: 2 - DNA, 750 bp, Synthetic construct
  source 1..750: mol_type = other DNA
tcctatgtgctgactcagtcaccctcactgtctgggtccctgggccagaaggtcaccatctcctgcactggaagtcgttccaatctcggtggaaattatgtgggctggtaccaacaagtcccaggaatggcccctagaaatgtcatatatgctaatgattttcgaccttcacggattcccgctcgattctccgcctccaagtcaggcacttcagccaccctgaccatctctgggctccaggctgaggacgaggctgactatttttgttcatcatgggatgatgatctcgggagtcacgtgttcggctcagggacccagctgaccatcctcggtggtggttcctctagatcttcctcctctggtggcggtggctcgggcggtggtggggaggagcaactggtggagtttgggggagacttggtgaagcctggggggtccctgagactctcctgtgtagcctctggattcagtttcagtagctacgacatgagctgggtccgccaggctcctgggaaggggctgcaatgggtcgcatctactaactttgatggaagtcgcacatactacactgacgctgtggagggccgattcaccatctccagagacaacgccaggaacacagtgtatctgcagatgaacagcctgagagccgaggacacggctgtgtattactgtgcgagggggatgggtagttggggcgcttcgacctttgggtactggggccagggaaccctggtcaccgtctcctca

SEQ ID NO: 3 - DNA, 741 bp, Synthetic construct
  source 1..741: mol_type = other DNA
cagtctgtgctgaatcagccaccttctgtgtctggagccctggggcagaccgtcaccatctcctgctctggaagtcacaccaacatcggcagtggatttgatgtacagtggtaccaacagctcccaggaaagtctcctcaaactctcgtctatgctaatatcaatcgaccctcgggggtcccggctcgattctctggctccaagtcaggcagcacagccaccctgaccatcactggggtccaggctgaagatgaggctgattattactgccagtcgtatgatgacaacttcgatgggcacgtgttcggctcaggaacccaactgaccgtcctcggcggtggttcctctagatcttcctcctctggtggcggtggctcgggcggtggtggggaggtccagctggtgcagtctgggggagacctggtgaaacctgggggatccctgagactgtcctgtgtggcctctggattcaccttcagtaactacaccatgagctgggtccgccaggctccagggcaggggctgcagtgggtcgcatacattaactatgatgggagtatcacatactacgcagaggctgtgaagggccggttcaccgtctccagagacaacgccaagaacacgctgtatctgcagatgaacagtctgagagccgaggacacggccgtgtatttctgtgtgaaggaaactttcatgataccaactgagtggggccagggaacccttatcaccgtctcctca

SEQ ID NO: 4 - DNA, 744 bp, Synthetic construct
  source 1..744: mol_type = other DNA
cagtctgtgctgaatcagccgccttctgtgtctggagccctggggcagaccgtcaccatctcctgttctggaagtcacaccaacatcggcagtggatttgatgtacagtggtaccaacagctcccaggaaagtcccctcaaactatcatctatgctaatatcaatcgaccctcgggggtcccggctcgattctctggctccaagtcaggcagcacagccaccctgaccatcactggggtccaggctgaagatgaggctgattattactgccagtcgtatgatgacaatttcgattacgtgttcggctcagggacccaactgaccgtccttggtggtggttcctctagatcttcctcctctggtggcggtggctcgggcggtggtggggaggagcaactggtggagtttgggggagacctggtgaagcctggagggtccttgagactgtcctgtgtggtctctggattcgatttcagtcgctacggcatgtcctgggtccgtcagtctccagggaagggtctccagtgggtcgcagatatcagttacaggggaactacattttacgcagacgatgtgaagggccgatttaccgtctccagagacagtgccaagaacacactctacctcgaaatgaccagcctgacagtcgaggacacggccgtatattattgtgcgaatagtgccaactggtatgtctccccttttgaccactggggccagggaaccctggtcaccgtctcctca

SEQ ID NO: 5 - DNA, 738 bp, Synthetic construct
  source 1..738: mol_type = other DNA
cagcctgtgctgacccagccaccctcagtgtcggggtcccttggccagagggtcaccatctcctgttctggaagcacgaacaccatcggtgttgttggtgcgaactggtaccaacagctcccaggaaagtcccctaaactcctcgtgtacagtgatggggttcgaccgtcaggggtccctgaccggttttccggctccaggtctgccaactcagacaccctgaccatcactgggcttcaggctgaggacgaggctgattattactgccagtcctttgattccacacataatgccattgtattcggcggaggcacccatctgaccgtcctcggtggtggttcctctagatcttcctcctctggtggcggtggctcgggcggtggtggggaggtgcagctggtggagactgggggagacctggtgaagcctgcggggtccctgagactgtcgtgtgcggcctctggattccccttcagtggctacagtatgacctgggtccgccaggcccctgaaaagggtctgcaattggtcgcaggtattaacagcgatggaagtcacacatactacacagacgatgtgaagggccgattcaccatctccagagacaacaccaaatacatactgtatctgcaaatgaacagtctgagagccgaggacacggccatgtattattgtggctctcatagttggggcaattttcactactggggccagggaaccctggtcaccgtctcctca

SEQ ID NO: 6 - DNA, 747 bp, Synthetic construct
  source 1..747: mol_type = other DNA
cagcctgtgctcactcagccgccctcagtgtctgggtccctgggccagagggtcaccatctcctgcaccggaactaataccaacatcggcagtggttatagtgtacaatggtaccagcagctcccaggagagtccccaaaacctatcatttatggtagtagtaatcgaccctcgggggtcccggctcgattctctggctccaagtcaggcagcacaggcaccctgaccatcactgggatccaggctgaggatgaggctgattaccactgccagtcctatgatgacagcctcgatggtcatgctgtattcggcggagggacccagctgaccatcctcggtggtggttcctctagatcttcctcctctggtggcggtggctcgggcggtggtggggaggtccagctggtgcagtctgggggagacttggtgaagcctggggggtccttgagactgtcctgtgtggcctctggattcaccttcagtgacaattacatgagctggatccgtcaggttccagggaagggcctgcagtgggtctcacgaattagcactgatggaagaggcacaagctacgcagacgctgtgaagggccgattcaccatctccagagacaatgccaagaacacgctgtatctgcagatgaacagcctgagagacgaggacacggccgtgtattactgtgcaaggcggtctggtatttgggggtttgactactggggccagggcgccctggtcaccgtctcctca

SEQ ID NO: 7 - DNA, 753 bp, Synthetic construct
  source 1..753: mol_type = other DNA
tcctatgagctgactcagccaccatctatgtctgcagccctggggcagagggtcaccatctcctgcactggaagtaacaccaacatcggcagtggttatgatgtacaatggtaccaacaggtcccaggaaagtcccctacacctctgatttatggtaataccaatcgaccctcgggggtcccggtgcgattctctggctccaagtcaggcaacacagccaccctgaccatcactgggatccaggctgaggatgaggctgattattactgccagtcctatgatgacaatttcgatggtcatcacgtgttcggctcagggacccagctgaccgtcctcggtggtggttcctctagatcttcctcctctggtggcggtggctcgggcggtggtggggaggtacagctggtggaatctgggggagacctggtgaagcctgcggggtccctgagactgtcctgtgtggcctctggattcaccttcagtaactatggcatgaattgggtccgccaggctcccgggaaggggctgcagtgggtcgcaggtattaacagtggtggaagtaccacaacctacgcagacgttgtgaagggtcgattcaccatctccagagacaacgccaagaacacactgtatctgcagatgaacagcctgggagccgaggacacggccgtgtattactgtgcgagggtggattatagtagcagctggttggttgactactggggccagggaaccctggtcaccgtctcctca

SEQ ID NO: 8 - DNA, 741 bp, Synthetic construct
  source 1..741: mol_type = other DNA
cagtctgtgttgactcaaccggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgaatattattgttcatcctgggatgatagtctcagaggtaccgtgttcggctcagggacccaactgaccgtccttggcggtggttcctctagatcttcctcctctggtgacggtggctcgggcggtggtggggaggtacagctggtggaatctgggggagacctggtgaagcctggggaatccctgagactgtcctgtgtggcctctggattcaccttcagtagctatggcatgagctgggtccgacagtctccagggaaggggttgcagtgggtcgcagatattagttatggaggaggtacatactacgcagacgctgtgaagggccgattcaccatctccagagacaacgccaggaacacagtatatctgcagatgaacagcctgagagccgaggacacggccgtgtattactgtgcgtcccaatggggtgattggggccactttgagtactggggccagggcaccctggtcaccgtctcctca

SEQ ID NO: 9 - DNA, 750 bp, Synthetic construct
  source 1..750: mol_type = other DNA
cagcctgtgctgacccagccaccctctgtgtctgcagccctgggacagagggtcaccctctcctgcactggaagtaagaccaacatcggcagtggttatgatgtacaatggtaccagcagttcccaggaaagtcccctaaaaccatcatctatggtaatagcaatcgattttcgggggtcccggctcgattctctggctccaagtcaggcagcatcgccaccctgaccatcactgggatccaggctgaggatgaggctgattattactgccagtcctttgatgacaatgtcgatggttacgtgttcggctcagggacccagctgaccatcctcggtggtggttcctctagatcttcctcctctggtggcggtggctcgggcggtggtggggaggtacagctggtggagtctgggggagacctggtgaagcctggggggtccctgagactctcctgtgtggcctctggattcaccttcagtagcgacagcatacattgggtccgtcaggctccaggaaaggggctgcagtgggtcgcatacattaacagtggtggaagtaccacatactacgcagacgctgtgaagggccgattcaccatctccagagacaacgccaggaacacagtgtatctgaagatgaacagcctgagagccgaggacacggctgtgtattactgtgcgaaagattcaggctttggcagctggtaccttgaatcctggggccagggaaccctggtcaccgtctcccca

SEQ ID NO: 10 - DNA, 741 bp, Synthetic construct
  source 1..741: mol_type = other DNA
caggctgtgctgaatcagccggcctctgtgtctggagccctggggcagaccgtcaccatctcctgttctggaagtcacaccaacatcggcagtggatttgatgtacagtggtaccaacagctcccaggaaagtcccctcaaactatcatctatgctaatatcaatcgaccctcgggggtcccggctcgattctctggctccaagtcaggcagcacagccaccctgaccatcactggggtccaggctgaagatgaggctgattatcactgccagtcctttgatgacgacctcgatggtcctgtgttcggcggaggcacccatctgaccgtcctcggtggtggttcctctagatcttcctcctctggtggcggtggctcgggcggtggtggggaggtccagctggtgcagtctggaggagacctggtgaagcctggggggtccctgaaaatttcctgtgtggcctctggactcaccttcaatagaaactacatgacctggatccgccaggctccagggaaggggctgcagtgggtctcagaaattaaccctgatggaagtaccacgagttacacagacgctgtgaggggccgattcaccatctccagagacaatgccaagaacacgttgtatctgcagatgaatagtatgagagatgaggacacggcagtgtattactgcgcaaggagtagttggggttcttttgactattggggccagggaaccctggtcaccgtctcctca

SEQ ID NO: 11 - DNA, 729 bp, Synthetic construct
  source 1..729: mol_type = other DNA
cagcctgtgctgacccagccaccctctgtgtctggagccctggggcagaccgtcaccatctcctgctctggaagtcacaccaacatcggcagtggatttgatgtacagtggtaccaacagctcccaggaaagtcccctcaaactctcgtctatgctaatatcaatcgaccctcgggggtcccggctcgattctccggctccaagtcaggcagcacagccaccctgaccatcactggggtccaggctgaagatgaggctgattattactgccagtcgtatgatgacaatttcgattacgtgttcggctcagggacccaactgaccgtccttggtggtggttcctctagatcttcctcctctggtggcggtggctcgggcggtggtggggaggtacagctggtggaatctgggggagacctggtgaagcctggggggtccttgagactgtcctgtgtggcctctggattcaccttcagtgactatagcatgagctgggtccgtcagtctccagggaaggggctgcagtgggtcgcagctattatgactagtggaagcacatactacgcagacgctgtgaagggccgattcaccatctccagagacaacgccaagaatacgctgtatctgcagatggacagcctgagagccgaggacacggccgtttattactgtgcgcacgactactggtttgacttctggggccagggcaccctggtcaccgtctcctca

SEQ ID NO: 12 - DNA, 741 bp, Synthetic construct
  source 1..741: mol_type = other DNA
tcctatgagctgactcagccaccatcagcgactgtgaccctgaggcagacggcccgcctcacctgtgggggagagaacattggaagaaaaagtgttcaatggtaccaacagaagccgggccagtcccccctactcattatctatgctgattccagcaggccgtcagggatccctgagcgattctctggcgccaattcggggaacacggccagcctgaccatcagcggggccctggccgaggacgaggctgactattactgccagatttgggaccgcaatattaatgtgttcggctccgggacccagctgaccgtccttggcggtggttcctctagatcttcctcctctggtggcggtggctcgggcggtggtggggaggagcaactggtggagtttgggggagacctggtgaagcctgcggggtccctgagactgtcctgtgtggcctcgggattcaccttcagtagctacgacatgagctgggtccgccaggctcctgggaaggggctgcagtgggtcgcgactattagttatgatggaagtagtacatactacactgacgctgtgaagggccgattcaccgtctccagagacaacgccaagaacacgttgtatctgcagatgaacagcctgagagccgaggacacggctctgtattactgtgcaaaggattctggcagttggtcccatttctatggtttggacacctggggccatggcacctcagtcttcgtgtcttcc

SEQ ID NO: 13 - DNA, 738 bp, Synthetic construct
  source 1..738: mol_type = other DNA
cagcctgtgctgacccagccaccctctgtgtctggagccctggggcagaccgtcaccatctcctgctctggaagtcacaccaacatcggcagtggatttgatgtacagtggtaccaacagctcccaggaaagtcccctcaaactctcgtctatgctaatatcaatcgaccctcgggggtcccggctcgattctctggctccaagtcaggcagcacagccaccctgaccatcactggggtccaggctgaagatgaggctgattattactgccagtcgtatgatgacaatttcgattacgtgttcggctcagggacccagctgaccgtcctcggcggtggttcctctagatcttcctcctctggtgacggtggctcgggcggtggtggggaggtacagctggtggagtctgggggaggcctgatgaagcctggggggtccctgaaactctcctgtttggcctctggtttcaccttcagtcgctactatatgtattgggtccgccaggctccagggaagggacttcagtgggtcgcacaaattagcagtgatggcagaagaacatactacgcagacgctgtgcagggccgattcaccatctccagagacaatgtcaagaacacggtatatctgcagatgaacagcctgagcgacgaggacacggccgtgtattattgtgtccgagaggactacttcgactttgactactggggccagggcaccctggtcaccgtctcctca

SEQ ID NO: 14 - DNA, 744 bp, Synthetic construct
  source 1..744: mol_type = other DNA
cagtctgtgctgaatcagccaccttctgtgtctgcggccctggggcagaggatcaccatctcctgcactggaagtcacaccaacatcggcagtggttatgatgtacaatggtaccagcagctccccggaaagtcccctagaactatcatttatggtaatagcgatcggccctctggggtcccggttcgattctctggctccaagtcaggcaacacagccaccctgaccatcactgggatccaggctgaggatgaggctgattattattgccagtcctatgatgacaacctcaatgatccttacgtgttcggctcagggacccaactgaccgtccttggtggtggttcctctagatcttcctcctctggtggcggtggctcgggcggtggtggggaggtccagctggtgcagtctgggggagacctggtgaagcctggggggtccctgagactctcctgtgtggcctctggattcgccttcagtggcaaaacaatgcaatgggtccgtcaggctccagggaaggggctacagtgggtcgcatacattcaacatgatgggagtagaacatcctacgcagacgctgtgaagggccgattcaccatctccagagacaacgccaagaacacgctctatctccagatggacagcctgagagccgaggacacggccgtgtattactgtgcggatcatccctggtacgggcctgagtattggggccagggaaccccggtcaccgtctcctca

SEQ ID NO: 15 - DNA, 756 bp, Synthetic construct
  source 1..756: mol_type = other DNA
tcctatgagctgactcagccaccatcggtgtctgcagccctggggcagggggtcaccatctcctgcatcggcagtgacaccaacatcggcaatcattatgatgtacaatggtaccagcagctcccaggaaagtcccctagaactattatttatggtaatagcgatcgaccctcgggggtcccgggtcgattctctggctccaagtcaggcaacacagccaccctgaccatcactgggatccaggctgaggatgaggctgattatcactgccagtcctatgatgacgacctcgatggccatattgtgttcggcggaggcacccatctgaccgtcctcggtggtggttcctctagatcttcctcctctggtggcggtggctcgggcggtggtggggaggtccagctggtgcagtctgggggagacctggtgaagcctgggggttccctgagactgtcctgtgtggcatctggattcaccttcaataagtaccacatgagttgggtccgccaggctccagggaaggggctgcagtgggtcggaaagataaccagtgatggaagtgccacagggtatgcagacgctgtaaagggccgattcaccatctccagagacaatgccaagaacacgttctatctacagatggacagcctgagagccgaggacacggctatgtattactgtgcaagggatcagagtagttggggaacgggtaattttgactactggggccagggaaccctggtcaccgtctcctca

SEQ ID NO: 16 - DNA, 741 bp, Synthetic construct
  source 1..741: mol_type = other DNA
cagtctgtgctgactcagccgacctcagtgtctgggtccctgggccagagggtcaccatctcctgcactggaagcagctccaatgttggttatggcaattatgtgggctggtaccagcagctcccaggaacaggccccagaaccctcatctatggtagtcattaccgaccctcgggggtccctgatcgattctctggctccagttcaggcagctcagccacactgaccatctctgggctccaggctgaggatgaggctgattattactgctcatcctacgacagcactctcagtggttacgtgtttggctcagggacccagctgaccatcctcggcggtggttcctctagatcttcctcctctggtggcggtggctcgggcggtggtggggaggtacagctggtggaatctgggggagacctggtgaagcctggggggtccctgagactctcctgtgtgggctctggattcaccttgagtagttattggatgagctgggtccgccaggctccagggaaggggctacagtggattgcagaaatcagcggcagtggaaataccatatattatggggacgctgtgaagggccgattcaccatctccagagacaatgccaagaacacgctgtatctgcagatgaacagcctgagagatgaggacacggcagtgtattactgtgcaacggaatactactggaatattgacaactggggccagggaaccctggtcaccgtctcctca

SEQ ID NO: 17 - DNA, 747 bp, Synthetic construct
  source 1..747: mol_type = other DNA
cagcctgtgctgacccagccaccctctgtgtctgcagccctggggcagagggtcaccatctcctgcactggaagtaagaccaacatcggcagtggttatgatgtacaatggtaccagcagctcccaggaaagtcccctaaaaccatcatttatggtaatagcaatcgaccctcgggggtcccggctcgattctctggctccaagtcaggcagcatcgccaccctgaccatcactgggatccaggctgaggatgaggctgattattactgccagtcctttgatgacaatgtcgatggttacgtgttcggctcagggacccagctgaccatcctcggtggtggttcctctagatcttcctcctctggtggcggtggctcgggcggtggtggggaggtgccactggtggaatctgggggagacctggtgaagcctgggggatccctgagactgtcctgtgtggcctctggattcaccttcagtagctacggcatgagctgggtccgccaggctccagggaagggactacagtgggtcgcatacattaatggtggtggaagtagtacatcctacgcagacgctgtgaggggccgattcaccatctccagagacaacgccgagaacacgctgtatctgcagatgaacagcctgagagccgacgatacggctgtatattactgttcgaattactacggtatctactgggattctgactactggggccagggaaccctggtcaccgtctcctca

SEQ ID NO: 18 - DNA, 741 bp, Synthetic construct
  source 1..741: mol_type = other DNA
cagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctca

SEQ ID NO: 19 - DNA, 738 bp, Synthetic construct
  source 1..738: mol_type = other DNA
caggctgtgctgactcagccggcctcagtgtcggggccccttggccagagggtcaccatttcctgctctggaagcacgaacaacatcggtattgttggtgcggcctggtaccaacagtttccaggaaaggcccctaaactcctcgtatacagtgatgggagtcgaccgtcaggggtccctgaccggttttccggctccaactctggcaactcagccaccctgaccatcagtggacttcaggctgaggacgaggctgattattactgtcagtctgttgatgccacgcatggtactattctgttcggcggagggacccagctgaccgtcctcggtggtggttcctctagatcttcctcctctggtggcggtggctcgggcggtggtggggaggtgcagctggtggagactgggggagacctggtgaagcctgcggggtccctgagactgtcatgtgcggcctctggattccccttcagtggctacagtatgacctgggtccgccaggcccctgaaaagggtctgcaattggtcgcaggtattaacagcgatggaagtcacacatactacacagacgatgtgaagggccgattcaccgtctccagagacaacaccaaatacatactgtatctgcagatgaacagtctgagagtcgaggacacggccatatattattgtggctctcatagttggggcaattttcactactggggccagggaaccctggtcaccgtctcctca

SEQ ID NO: 20 - DNA, 726 bp, Synthetic construct
  source 1..726: mol_type = other DNA
cagtctgtgctgaatcagccaccttcagtatcagtctctctgggacagacagcaaccatctcctgctctggagagagtctgagtaaatattatgcacaatggttccagcagaaggcaggccaagtccctgtgttggtcatatataaggacactgagcggccctctgggatccctgaccgattctccggctccagttcagggaacacacacaccctgaccatcagcggggctcgggccgaggacgaggctgactattactgcgagtcagaagtcagtcctgatactattgtgttcggcggaggcacccacctgaccgtcctcggtggtggttcctctagatcttcctcctctggtggcggtggctcgggcggtggtggggaggtacagctggtggaatctgggggagacctggtgaagcctgggggatccctgagactgtcctgtgtggcctctggattcaccttcagtagctatgccatgagctgggtccgccaggctccaggggaggggctgcagtgggtcgcatacattaacagtgctggaagtaccacatactacgcagacgctgtgaagggccgattcaccatctccagagacaacgccaggaacacagtgtatctgcagatgaacagcctgagagccgaggacacagccgtgtattactgtggccgatattattatggtctggactactggggtcctggcacctcagtcttcgtgtcctca

SEQ ID NO: 21 - DNA, 732 bp, Synthetic construct
  source 1..732: mol_type = other DNA
cagcctgtgctgacccagccaccctcggtgtcagtgtctctgggacagacagcaaccatctcctgctctggagagagtctgagtaaatattatgcacaatggttccagcagaaggcaggccaagcccctgtgttggtcatatataaggacactgagcggccctctgggatccctgaccgattctctggctccagttcagggaacacacacaccctgaccatcagcggggctcgggccgaggacgaggctgactattactgcgagtcagcagtcagtagtgatactgcagtgttcggctcagggacccaactgaccgtcctcggcggtggttcctctagatcttcctcctctggtggcggtggctcgggcggtggtggggaggtacagctggtggagtctgggggagatatggtgaagcctggggggtccttgagactgtcctgtgtggcctctggattcaccttcagtagtttctggatgtactgggtccgccaggctcctgggaaggggctgcagtgggtcggaggtattaatagtggtggaagtattacaagttacgcagacgctgtgaagggccgattcaccatctccagagacaacgccaggaacacactgtatctgcagatggacagtctgagagatgaagacacagccgtctactactgtgcgaagccggatatatatgggagttttgaccactggggccagggcaccctggtcaccgtctcctca

SEQ ID NO: 22 - DNA, 741 bp, Synthetic construct
  source 1..741: mol_type = other DNA
cagcctgtgctcactcagccgccctcagtgtctgggtccctgggccagagggtcaccatctcctgcactggaagcagctccaatgttggttatggcgattatgtgggctggtaccagcagctcccaggaacaagccccagaaccctcatttattctagtagtagccgaccctcgggagtccccgatcgattctctggctccaggtcaggcagcacagcaaccctgaccatctctgggctccaggctgaagatgaagccgattatcactgctcatcctatgacaccagtctcaatggtttcgtgttcggctcagggacccagctgaccatcctcggtggtggttcctctagatcttcctcctctggtggcggtggctcgggcggtggtggggaactcacactgcaggagtcagggccaggactggtgaagcccgcacagaccctctctctcacctgtactgtgtccggagactccgtcaccagcagtcacttctggcactggatccgccagcgccctgggaggggactggaatggatgggatactggtcagacattacgatttacagtccaacattccaaggacgcatctccatcactgttgacacggccaggaaccagttctccctgcatttgaactccgtgacgaccgaggactcggccgtttatttctgtgcaagatataatggatacgtcgattatttagacgtctggggccagggcaccctgatcaccgtctcctca

SEQ ID NO: 23 - DNA, 735 bp, Synthetic construct
  source 1..735: mol_type = other DNA
gatattgtcatgacacagacgccactgtccctgtccgtcagtcctggagagccggcctccatctcctgcaaggccagtcagagcctcctgcacagtaacgggaacacctatttgtattggttccgacagaagccaggccagtctccacagcgcctgatctacttggtttccaacagagacgctggggtcccagacaggttcagtggcagcgggtcagggacagatttcaccctgagaatcagcagagtagaggctgatgattctggaatttattactgcggccaaggtaaacaagatcctatcacctttggcgaagggacacatctggagattaaaggtggttcctctagatcttcctcctctggtggcggtggctcgggcggtggtggggaactcacactgcaggagtcagggccaggactggtgaagccctcacagaccctctctctcacctgtgttgtgtccggaggctccgtcaccaccagtgactactggaactggctccgccagcgccctgggaggggactggaatacatgggatactggaaaggtcggactgagtacaacccggcattccagggacgcatctccatcactgctgacacggccaagaaccagttctccctgcagttaaactccgtgaccgccgacgacacggccatttattattgtgcacattgggggtcaggcaatcttgactattggggcccgggaaccctggtcaccgtctcctca

SEQ ID NO: 24 - DNA, 741 bp, Synthetic construct
  source 1..741: mol_type = other DNA
cagtctgtgctgaatcagccaccttctgtgtctggagccctggggcagaccgtcaccatctcctgctctggaagtcacaccaacatcggcagtggatttgatgtacagtggtaccaacagctcccaggaaagtcccctcaaactctcgtctatgctaatatcaatcgaccctcgggggtcccggttcgattctctggctccaagtcaggcaccacagccaccctgaccatcactgggatccaggctgaggatgaggctgattattactgccagtcctatgatgacaacgtcgatggttacgtgttcggctcagggacccagctgaccgtccttggtggtggttcctctagatcttcctcctctggtggcggtggctcgggcggtggtggggaggtccagctggtgcagtctgggggaracctggtgaagcctggggggtccctgagactctcctgtgtggcctctggattcaccctcagtagtaactatatggactgggtccgccaggctccagggaaggggcctcagtgggtcgcacaaattagcactgatggaagtaggacatattacgcagacgctgtgaagggccgattcaccatttccagagacaccgccaagaacacactgtatctgcagatgaacagtctgagagccgaggacacggctatgtattactgtgcaaagaacgaatacttggcccctacgtactggggccagggcaccctggtcaccgtctcctca

SEQ ID NO: 25 - DNA, 750 bp, Synthetic construct
  source 1..750: mol_type = other DNA
gatatcgtcatgacacagactccgctgtccctgtccgtcagccctggagagacggcctccatctcctgcaaggccagtcagagcctcctgcacagtaacgggaacacctatttgaattggttccgacagaagccaggccagtctccacaggtcctgatctatcaggtctccaacagagacactggggtcccagacaggttcagtggcagcgggtcagggacagatttcaccctgagaatcagtagagtggaggctgacgatgctggagtttattattgcgggcaaggtatacaatatcctccgacgttcggagcaggaaccaaggtggagctcaaaggtggttcctctagatcttcctcctctggtggcggtggctcgggcggtggtggggaggagcaactggtggagtttgggggagacctggtgaagcctggggggtccttgagactctcctgtgtggcctctggattcaccttcagtagtctttccatgaactggatccgccaggctccagggaaggggctgcagtgggtctcacaaattagccctgatggaactatcacaggctacacagacgctgtgaagggccgattcaccatctccagagacaatgccaagaacacgctgtatcttcagatgaacagcctgagagatgaagacacggcaatatattactgtgcgaagagtcgatatacggatacgtacccctttgactactggggcccgggaaccctggtcaccgtctcctca

SEQ ID NO: 26 - DNA, 723 bp, Synthetic construct
  source 1..723: mol_type = other DNA
tccagtgtgctgactcagcctccctcggtatcagtgtctctgggacagacagcaaccatctcctgctctggagagagtctgagtaaatattatactcaatggttccagcagaaggcaggccaagcccctatcttggtcatttataaggacactgagcggccctctgggatccctgaccggttctctggctccagttcaggcaacacacacaccctgaccatcagcggggctcgggccgaggacgaggctgactattattgcgagtcattagtcgattctgatgcttacatgttcggctctggaacccagctgaccgtccttggcggtggttcctctagatcttcctcctctggtggcggtggctcgggcggtggtggggaggtgcagctggtggagactgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 27 - DNA, 726 bp, Synthetic construct
  source 1..726: mol_type = other DNA
cagcctgtgctgacccagccaccctcggtatcagtgtctctgggacagacagcaaccatctcctgctctggagagagtctgagtaaatattatgcacaatggttccagcagaaggcaggccaagcccctgtgttggtcatatataaggacactgagcggccctctgggatccctgaccgattctctggctccagttcagggaacacacacaccctgaccatcactgggatccaggctgaggatgagggtgattatcactgccactcctatgatgagaacctcgatggttacgtgttcggctcaggaacccaactgaccgtccttggcggtggttcctctagatcttcctcctctggtggcggtggctcgggcggtggtggggaggtgcagctggtggagtctgggggagacctggtgaagcctggggggtctctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 28 - DNA, 723 bp, Synthetic construct
  source 1..723: mol_type = other DNA
cagtctgccctgactcaaccttcctcggtatcagtgtctctgggacagacagcaaccatctcctgctctggagagagtctgaataaatattatgtacagtggttccagcagaaggcaggccaagtccctatattggtcatatatagggacactgagcggccctctgggatccctgaccgattctctggctccagttcagggaacacacacaccctgaccatcaccgggactcgggccgaggacgaggctgactattactgcgagtcactagtcaatgataatacttacgtgttcggctcagggacccaactgaccgtccttggcggtggttcctctagatcttcctcctctggtggcggtggctcgggcggtggtggggaggtgcagctggtggagactgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 29 - DNA, 723 bp, Synthetic construct
  source 1..723: mol_type = other DNA
cagtctgtgctgaatcagccgccttcagtatcagtgtctctgggacagacagcaaccatctcctgctctggagagagtctgaataaatattatacacagtggttccagcagaaggcaggccaagcccctgtgttggtcatatataaggacactgagcggccctctgggatccctgaccgattctccggatccagttcagggaacacacacaccctgaccatcagcggggctcgggccgaggacgaggctgactattactgcgagtcagtagacagtactgatacttacgtgttcggctcagggacccagctgaccatcctcggtggtggttcctctagatcttcctcctctggtggcggtggctcgggcggtggtggggaggtacatctggtggaatctgggggagacttggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 30 - DNA, 726 bp, Synthetic construct
  source 1..726: mol_type = other DNA
cagtctgtcttgactcagccagcctcggtatcagtgtctctgggacagacagcaaccatctcctgctctggagagagtctgagtaaatattatacacagtggttccagcagaaggcaggccaggcccctgtgttggtcatctataaggacactgagcggccctctgggatccctgaccgattctctggctccagctcagggaacacgcacaccctgaccatcagcggggctcaggccgaggatgaggctgattattactgccagtcctatgatgacaacgtcgatggttacgtgttcggctctgggacccaactgaccgtcctcggtggtggttcctctagatcttcctcctctggtggcggtggctcgggcggtggtggggaggtacagctggtggaatctgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 31 - DNA, 723 bp, Synthetic construct
  source 1..723: mol_type = other DNA
cagtctgccctgactcaaccttcctcggtatcagtgtctctgggacagacagcaaccatctcctgctctggagagagtctgactaaatattatgtacaatggttccaacagaaggcaggccaagcccctgtattggtcatatatagggacactgagcggccctctgggatccctgaccgattctctggctccagttcaggtaatacacacaccctgaccatcagtggggctcgggccgaggacgaggctgactattattgcgagtcaatggacggtcgtgaggcttacgtgttcggctcagggacccaactgaccgtcctcggcggtggttcctctagatcttcctcctctggtggcggtggctcgggcggtggtggggaggtacagctggtggagtctgggggagacctggtgaagccgggggggtccctgagactgtcctgtgtggcctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 32 - DNA, 726 bp, Synthetic construct
  source 1..726: mol_type = other DNA
cagtctgtgttgactcaaccagcctcggtatcagtgtctctgggacagacagcaaccatctcctgctctggagagagtctgagtaaatattatgcacaatggttccagcagaaggcaggccaagcccctgtgttggtcatatataaggacactgagcggccctctgggatccctgaccgattctctggctccagttcagggaacacacacaccctgaccatcactgggatccaggctgcggatgaggctgattattactgccagtcctatgatgacaacctcgatggttacgtgttcggctcaggaacccagctgaccgtccttggtggtggttcctctagatcttcctcctctggtggcggtggctcgggcggtggtggggaggtacagctggtggagtctgggggagacctggtgaagcctggggggtccctgagactctcctgtgtgggctctggattcaccttcagtagctactggatgagctgggtccgccaggctccagggaaggggctacagtggattgcagatattagcgatagtggaagtagcacagactatgcagacgctgtgaagggccgattcaccatctccagagacaatgccaagaacacactgtatctgcagatgaacagcctgagagccgaggacacggccatgtattactgtgcaaggggtctgggtatggactactggggccatggcacctcactcttcgtgtcctca

SEQ ID NO: 33 - DNA, 729 bp, Synthetic construct
  source 1..729: mol_type = other DNA
gatattgtcatgacacagacgccactgtccctgtccgtcagccctggagagccggcctccatctcctgcaaggccagtcagagcctcctgcacagtgacgggaacacctatttgcattggtacctgcaaaagccaggccagtctccacagcttctgatctactcggtttccaaccgcttcactggcgtgtcagacaggttcagtggcagcgggtcagggacagatttcaccctgagaatcagcagagtggaggctgacgatactggagtttattactgcgggcaaggtacacacctcccgttcacttttggccaagggaccaaactggagatcaaaggtggttcctctagatcttcctcctctggtggcggtggctcgggcggtggtggggaggtacagctggtggagtctgggggagacctggtgaagcctggggggtccctgagactctcctgtgcagcctctggattcaccttcagtcactacgacatgaactgggtccgccaggctcctgggaaggggctgcagtgggtcgcatctattgcctatgatggatctaacacatactacactgacgctgtgaagggccgattcaccatctccagagacaacgccaagaatatactgtttttgctggtgaatagcctgagggccgaggacacggccatatattattgtgcgcgggggctggtctactggggccagggaactttggtcaccgtctcctca

SEQ ID NO: 34 - DNA, 747 bp, Synthetic construct
  source 1..747: mol_type = other DNA
tcctatgagctgactcagccaccatctgtgtctgcggccctgggacagagggtcaccatctcctgcactggaactaataccaacatcggcagtggttatagtgtacaatggtaccagcagctcccaggagagtccccaaaacctatcatttatggtagtagtaatcgaccctcgggggtcccggctcgattctctggctccaagtcaggcagcacaggcaccctgaccatcactgggatccaggctgaggatgaggctgattaccactgccagtcctatgatgacagcctcgatggccatgctgtattcggcggagggacccagctgaccatcctcggcggtggttcctctagatcttcctcctctggtggcggtggctcgggcggtggtggggaggtacagctggtggagtctgggggagacctggtgaagcctggggggtccctgagactctcctgtgtggcctctggattaaccttcagtgacaattacatgtcctggatccgtcaggttccagggaagggcctgcagtgggtctcacgaattagcactgatggaagaggcacaagttacgcagacgctgtgaagggccgattcaccatctccagagacaatgccaagaacacgctgtatctgcagatgaacagcctgagagacgaggacacggccgtgtattactgtgcaaggcggtctggtatttgggggtttgactactggggccagggcaccctggtcaccgtctcctct

SEQ ID NO: 35 - DNA, 723 bp, Synthetic construct
  source 1..723: mol_type = other DNA
tccagtgtgctgactcagcctccttcggtatcagtgtctctgggacagacagcaaccatctcctgctctggagagagtctgagtgaatattatgcacaatggttccagcagaaggcaggccaagcccctgtattggtcatatataaggacactgagcgggcctctggaatccctgaccgattctctggctccagttcggggaacacacacaccctgaccatcagcggggctcgggccgaggacgaggctgactattactgcgagtcagcagtcaatagtgatggtattgtgttcggcggaggcacccatctgaccgtcctcggcggtggttcctctagatcttcctcctctggtggcggtggctcgggcggtggtggggaggtacagctggtggagtctgggggagacctggtgaagcctgcggggtccctgagactgtcctgtgtgacctctggattcaccttcagcagctatgccatgagctgggtccgccaggctcctgggaaggggctgcagtgggtcggaagtattaacagtggtggaagtaccacaagctacgcagacgctgtgaagggccgattcaccatctccagagacaacgccaagaacacagtgtatctgcagatgaacagcctgagagccgaggacacggccgtgtattactgtgcccggtggtgggactttgagtactggggccagggcaccctggtcaccgtctcctca

SEQ ID NO: 36 - DNA, 738 bp, Synthetic construct
  source 1..738: mol_type = other DNA
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtgtacagtgatggggatcgaccgtcaggggtccctgaccggttttccggctccaactctggcaactcagacaccctgaccatcactgggcttcaggctgaggacgaggctgattattactgccagtcctttgataccacgcttgatgcttacgtgttcggctcaggaacccagctgaccgtccttggtggtggttcctctagatcttcctcctctggtggcggtggctcgggcggtggtggggaggagcaactggtggagtttgggggaggcctggtgaggcctgggggatccctgagactgtcctgtgtggcctctggattcaccttcagtagctatggcatgacctgggtccgtcagtctccagggaaggggctgcagtgggtcgctactattagcgataccggaagcacatactacgcagacactgtgaagggccgattcagcatctccagagacaacgccaagaacacgctgtatctgcagatgaacagtctgagagccgacgacacggccctgtattattgtacgaaggacctcggggtgtggggtatggacaactggggtcctggcacctcactcttcgtgtcctca

SEQ ID NO: 37 - DNA, 720 bp, Synthetic construct
  source 1..720: mol_type = other DNA
tccagtgtgctgactcagcctccctcagtatcagtgtctctgggacagacagcaaccatctcctgctctggagagagtctgaatgactattatgcacaatggttccagcagaaggcaggccaagtccctgtgttggtcatatatcgggacactgagcggccctctgggatccctgaccgattctccggctccagttcagggaacacacacaccctgaccatcagcggggctcgggccgaggacgaggctgactattactgcgagtcagaagtcagtgctggtggtgctgtattcggcgaaggcacccacctgaccgtcctcggtggtggttcctctagatcttcctcctctggtggcggtggctcgggcggtggtggggaggtgcagctggtggagactgggggagacctggtgaagccgggggggtccctgagactgtcctgtgtggcctctggattcaccctcagagactacagcatgacctgggtccgccagactcctgagaaggggctgcagttggtcgcaggtattagcaccggtggaagtaccacatactacatagacgctgtgaagggccgattcaccatctccagagacaacgccaagaacacagtgtatctgcagatgaacagcctgagagccgaggacacggccatgtatcactgtacaaccacggccttaggttactggggccagggcaccctggtcactgtctcctca

SEQ ID NO: 38 - DNA, 723 bp, Synthetic construct
  source 1..723: mol_type = other DNA
tccagtgtgctgactcagcctccctcagtatcagtctctctgggacagacagcaaccatctcctgctctggagagagtctgagtgagtattatgcacaatggttccagcagaaggcaggccaagcccctgtgttggtcatatataaggacactgagcggccctctgggatccctgaccgattctccggctccagttcagggaacacacacaccctgaccatcagcggggctcgggccgaggacgaggctgactattactgcgcgtcagcagacagtagtgctattcctgtgttcggcggaggcacccatctgaccgtcctcggcggtggttcctctagatcttcctcctctggtggcggtggctcgggcggtggtggggaggtgcagctggtggagtctgggggagacctggtgaagcctggggggtccctgagactctcctgtgtggcctcaggattcaatttcggaaactatgccgtgagctgggtccgccaggctcctgggaagggactgcagtgggtcgcaggtatcagtaacgatggaactataacaggctacacagatgttgtggagggccgtttcatcatctccagagacaatgccaagaacacgctgtatctacagatgaacagcctgagagccgaggacatggctatatattattgtgtggctgggggctttcttgactactggggccagggaaccctggtcaccgtctcctca

SEQ ID NO: 39 - DNA, 747 bp, Synthetic construct
  source 1..747: mol_type = other DNA
cagtctgtgctgactcagccgccctcagtgtcggggtcccctggccagagggtcaccatctcctgctctggaaccacgaagaacatcggtatagtcggtgcgagttggtaccaacaactcccaggaaaggcccctaaactcctcgttcacagtgatgggtttcgaccgtcaggggtccctgaccggttttccgcctccaactctggcgactcagacattctgaccatcactgggcttcaggctgaggacgaggctgattattactgtcagtcctttgataccacgcttgatgctcacgtcttcggctcagggacccagctgaccgtccttggcggtggttcctctagatcttcctcctctggtggcggtggctcgggcggtggtggggaggtgccactggtggaatctgggggagacctggtgaagcctggggggtccttgagactgtcctgtgtggcctctggattcacctttagtagttatggcatgagctgggtccgtcagtctccagggaaggggctgcagtgggtcgcaggtattacctatgatggaagtaatacatactacactgacactgtgaggggccgattcaccatctccagagacaacgccaggaacacagtgtatctgcagatgaacagcctgagagccgaggacacagctgtgtattactgtgtggccagccccctctacggaacttatggtttcgactactggggccatggcacctcagtcttcgtgtcctca

SEQ ID NO: 40 - DNA, 738 bp, Synthetic construct
  source 1..738: mol_type = other DNA
cagtctgtgctaactcagccgccctcagtgtctgggtccctgggccagaaggtcaccatctcctgcactggaagcagttccagcgtcggtactcattatgtgggctggtaccaaatacccccaggaataggccctagaaccgtcatctataataatgattaccgaccttcaggggtccccgatcgattctctggctccaagtcaggcaattcagccaccctgaccatctctgggctccaggctgaggacgaggctgattattactgctcatcatgggatgacagtctcggagcttacgtgctcggctcagggacccagctgaccatcctcggtggtggttcctctagatcttcctcctctggtggcggtggctcgggcggtggtggggaactcacactgcaggagtcagggccaggactggtgaagccctcacagaccctctctctcacctgcgctgtgtccggaggctccgtcatcagaggttactactggacctggatccgccagcgccctgggaggggactggaatggatgggttactgggcagggagtacagattataacccggcattccagggacgcatcttcatcactgctggcacggccaagaatcagttctccctggagatgacttccatgaccacggacgacacggccatatattactgtgtcggatcccttcggggcggttggctctttgacaactggggccagggaaccctagtcaccgtctcctcc

SEQ ID NO: 41 - DNA, 723 bp, Synthetic construct
  source 1..723: mol_type = other DNA
cagcctgtgctcactcagccgccctcggtctcagtgtctctgggacagacagcaaccatctcctgctctggagagagtctgagtaaatattatgcacagtggttccagcagaaggcaggccaagcccctgtcttggtcatatataaggacactgagcggccctctgggatccctgaccgattctctggctccagttcagggaacacacacaccctgaccatcagcggggctcgggccgaggacgaggctgactattactgcgagtcagcagtcagtactgagactgttgtgttcggcggaggcacccatctgaccgtcctcggcggtggttcctctagatcttcctcctctggtggcggtggctcgggcggtggtggggaggtgcagctggtggagactgggggagacctggtgatgcctgcagggtccctgagactgtcttgtgtggcctctggattcaccttcagtagttctgtcatgagctgggtccgtcaggctcctgggaaggggctggagtgggtcgcaggtattaatggtggcggaagttttgcggcctacggagacgctgtgaagggccgattcaccatatccagagacaacgccaagaacacgctctatctgcagatgaacagcctgagagccgaggacacggccatgtattactgtgtggggggcggttatatggacttctggggtcctggcacctcactcttcgtgtcctca

SEQ ID NO: 42 - DNA, 732 bp, Synthetic construct
  source 1..732: mol_type = other DNA
cagtctgtgctgactcagccgacctcagtgtcggggtcccttggccagagggtcaccatttcctgctctggaagcacgaacgacatcggtattattggtgcggcctggtaccaacagctcccagggaaggcccctaaactcctcctgtacagtaataagaatcgaccgtcaggagtccctgaccgattttccggctccaactctggcatctcagccaccctgaccatcactgggcttcaggctgaggacgaggctgattattactgtcagtctgttgatcccgcgcttcatacttacgtgtttggctcagggacccagctgaccatcctcggcggtggttcctctagatcttcctcctctggtggcggtggctcgggcggtggtggggaggtgcagctggtggagattgggggagaccttgtgaaacctgaggggtccctgagactctcctgtgtggtctctggcttcaccttcagtggctacgacatgaactgggtccgccaggctccagggaaggggcttcagtgggtcgcatacattagcagtgatgggaggagaacagtttacacagacgctgtgaagggccgattcaccatctccagagacaatgccaagaacacgctgtatctgcagatgaacagcctgaaatctgaggacacagccgtatattactgtgtgaaggggggttggttggacttctggggccagggaaccctggtcaccgtctcctca

SEQ ID NO: 43 - DNA, 732 bp, Synthetic construct
  source 1..732: mol_type = other DNA
gatattgtcatgacacagaacccactgtccctggccgtcacccctggagagctggccactatctcctgcagggccagtcagagtctcctgcacagtgatggaaaatcctatttgaattggtacctgcagaagccaggccagactcctcggccgctgatttatgaggcttccaggcgtttctctggggtctcagacaggttcaatggcagcgggtcagggacagatttcacccttaaaatcagtagggtggaggctgaggatgttggagtttattactgtcagcaaggtctacattttcctccgacgttcggagcaggaaccaaggtggagctcaaaggtggttcctctagatcttcctcctctggtggcggtggctcgggcggtggtggggaggtacagctggtggaatctgggggagacctggtgaagcctggggggtccctgagactctcctgtgcagcctctggattcaccttcactgactacgacatgagttgggtccgccaggctcctgggaaggggctgcagtgggtcgcagctattagctatgatggaagtagcacatactacactgacgctgtgaagggccgattcaccatctccagagacaacgccaggaacacactgtatctgcagatgagcagcctgagagccgaggacacggccatgtattactgtgtagtctcgaatatgaacctctggggccatggcacctcagtcttcgtgtcctct

SEQ ID NO: 44 - protein, 250 aa, Synthetic construct
QAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVLGGGSSRSSSSGGGGSGGGGEVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSS

SEQ ID NO: 45 - protein, 250 aa, Synthetic construct
SYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTILGGGSSRSSSSGGGGSGGGGEEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSS

SEQ ID NO: 46 - protein, 247 aa, Synthetic construct
QSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVLGGGSSRSSSSGGGGSGGGGEVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSS

SEQ ID NO: 47 - protein, 248 aa, Synthetic construct
QSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVLGGGSSRSSSSGGGGSGGGGEEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSS

SEQ ID NO: 48 - protein, 246 aa, Synthetic construct
QPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVLGGGSSRSSSSGGGGSGGGGEVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSS

SEQ ID NO: 49 - protein, 249 aa, Synthetic construct
QPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSS

SEQ ID NO: 50 - protein, 251 aa, Synthetic construct
SYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVLGGGSSRSSSSGGGGSGGGGEVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSS

SEQ ID NO: 51 - protein, 247 aa, Synthetic construct
QSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVLGGGSSRSSSSGDGGSGGGGEVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSS

SEQ ID NO: 52 - protein, 250 aa, Synthetic construct
QPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTILGGGSSRSSSSGGGGSGGGGEVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSP

SEQ ID NO: 53 - protein, 247 aa, Synthetic construct
QAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVLGGGSSRSSSSGGGGSGGGGEVQLVQSGGDLVKPGGSLKISCVASGLTFNRNYMTWIRQAPGKGLQWVSEINPDGSTTSYTDAVRGRFTISRDNAKNTLYLQMNSMRDEDTAVYYCARSSWGSFDYWGQGTLVTVSS

SEQ ID NO: 54 - protein, 243 aa, Synthetic construct
QPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGGGGSGGGGEVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSS

SEQ ID NO: 55 - protein, 247 aa, Synthetic construct
SYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVLGGGSSRSSSSGGGGSGGGGEEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSS

SEQ ID NO: 56 - protein, 246 aa, Synthetic construct
QPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGDGGSGGGGEVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSS

SEQ ID NO: 57 - protein, 248 aa, Synthetic construct
QSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVLGGGSSRSSSSGGGGSGGGGEVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSS

SEQ ID NO: 58 - protein, 252 aa, Synthetic construct
SYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVLGGGSSRSSSSGGGGSGGGGEVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSS

SEQ ID NO: 59 - protein, 247 aa, Synthetic construct
QSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTILGGGSSRSSSSGGGGSGGGGEVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSS

SEQ ID NO: 60 - protein, 249 aa, Synthetic construct
QPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTILGGGSSRSSSSGGGGSGGGGEVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSS

SEQ ID NO: 61 - protein, 247 aa, Synthetic construct
QSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSS

SEQ ID NO: 62 - protein, 246 aa, Synthetic construct
QAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVLGGGSSRSSSSGGGGSGGGGEVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSS

SEQ ID NO: 63 - protein, 242 aa, Synthetic construct
QSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVLGGGSSRSSSSGGGGSGGGGEVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSS

SEQ ID NO: 64 - protein, 244 aa, Synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVLGGGSSRSSSSGGGGSGGGGEVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSS

SEQ ID NO: 65 - protein, 247 aa, Synthetic construct
QPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTILGGGSSRSSSSGGGGSGGGGELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSS

SEQ ID NO: 66 - protein, 245 aa, Synthetic construct
DIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIKGGSSRSSSSGGGGSGGGGELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSS

SEQ ID NO: 67 - protein, 247 aa, Synthetic construct
  SITE 140: note = misc_feature - Xaa can be any naturally occurring amino acid
QSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVLGGGSSRSSSSGGGGSGGGGEVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSS

SEQ ID NO: 68 - protein, 250 aa, Synthetic construct
DIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELKGGSSRSSSSGGGGSGGGGEEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSS

SEQ ID NO: 69 - protein, 241 aa, Synthetic construct
SSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 70 - protein, 242 aa, Synthetic construct
QPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 71 - protein, 241 aa, Synthetic construct
QSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 72 - protein, 241 aa, Synthetic construct
QSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTILGGGSSRSSSSGGGGSGGGGEVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 73 - protein, 242 aa, Synthetic construct
QSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 74 - protein, 241 aa, Synthetic construct
QSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVLGGGSSRSSSSGGGGSGGGGEVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 75 - protein, 242 aa, Synthetic construct
QSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 76 - protein, 243 aa, Synthetic construct
DIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIKGGSSRSSSSGGGGSGGGGEVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSS

SEQ ID NO: 77 - protein, 249 aa, Synthetic construct
SYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSS

SEQ ID NO: 78 - protein, 241 aa, Synthetic construct
SSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVLGGGSSRSSSSGGGGSGGGGEVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSS

SEQ ID NO: 79 - protein, 246 aa, Synthetic construct
QSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVLGGGSSRSSSSGGGGSGGGGEEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSS

SEQ ID NO: 80 - protein, 240 aa, Synthetic construct
SSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVLGGGSSRSSSSGGGGSGGGGEVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSS

SEQ ID NO: 81 - protein, 241 aa, Synthetic construct
SSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVLGGGSSRSSSSGGGGSGGGGEVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSS

SEQ ID NO: 82 - protein, 249 aa, Synthetic construct
QSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVLGGGSSRSSSSGGGGSGGGGEVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSS

SEQ ID NO: 83 - protein, 246 aa, Synthetic construct
QSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTILGGGSSRSSSSGGGGSGGGGELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSS

SEQ ID NO: 84 - protein, 241 aa, Synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVLGGGSSRSSSSGGGGSGGGGEVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSS

SEQ ID NO: 85 - protein, 244 aa, Synthetic construct
QSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTILGGGSSRSSSSGGGGSGGGGEVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSS

SEQ ID NO: 86 - protein, 244 aa, Synthetic construct
DIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELKGGSSRSSSSGGGGSGGGGEVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSS

SEQ ID NO: 87 - protein, 8 aa, Synthetic construct
GFTFSNHE

SEQ ID NO: 88 - protein, 8 aa, Synthetic construct
IYDSGSRT

SEQ ID NO: 89 - protein, 15 aa, Synthetic construct
AGGNSRGWTEFGMDY

SEQ ID NO: 90 - protein, 9 aa, Synthetic construct
NTNIGSPYD

SEQ ID NO: 91 - protein, 12 aa, Synthetic construct
QSYDDSLDGRVV

SEQ ID NO: 92 - protein, 8 aa, Synthetic construct
GFSFSSYD

SEQ ID NO: 93 - protein, 8 aa, Synthetic construct
TNFDGSRT

SEQ ID NO: 94 - protein, 14 aa, Synthetic construct
ARGMGSWGASTFGY

SEQ ID NO: 95 - protein, 8 aa, Synthetic construct
RSNLGGNY

SEQ ID NO: 96 - protein, 11 aa, Synthetic construct
SSWDDDLGSHV

SEQ ID NO: 97 - protein, 8 aa, Synthetic construct
GFTFSNYT

SEQ ID NO: 98 - protein, 8 aa, Synthetic construct
INYDGSIT

SEQ ID NO: 99 - protein, 10 aa, Synthetic construct
VKETFMIPTE

SEQ ID NO: 100 - protein, 9 aa, Synthetic construct
HTNIGSGFD

SEQ ID NO: 101 - protein, 11 aa, Synthetic construct
QSYDDNFDGHV

SEQ ID NO: 102 - protein, 8 aa, Synthetic construct
GFDFSRYG

SEQ ID NO: 103 - protein, 8 aa, Synthetic construct
ISYRGTTF

SEQ ID NO: 104 - protein, 12 aa, Synthetic construct
NSANWYVSPFDH

SEQ ID NO: 105 - protein, 10 aa, Synthetic construct
QSYDDNFDYV

SEQ ID NO: 106 - protein, 8 aa, Synthetic construct
GFPFSGYS

SEQ ID NO: 107 - protein, 8 aa, Synthetic construct
INSDGSHT

SEQ ID NO: 108 - protein, 10 aa, Synthetic construct
GSHSWGNFHY

SEQ ID NO: 109 - protein, 8 aa, Synthetic construct
TNTIGVVG

SEQ ID NO: 110 - protein, 11 aa, Synthetic construct
QSFDSTHNAIV

SEQ ID NO: 111 - protein, 8 aa, Synthetic construct
GFTFSDNY

SEQ ID NO: 112 - protein, 8 aa, Synthetic construct
ISTDGRGT

SEQ ID NO: 113 - protein, 11 aa, Synthetic construct
ARRSGIWGFDY

SEQ ID NO: 114 - protein, 9 aa, Synthetic construct
NTNIGSGYS

SEQ ID NO: 115 - protein, 12 aa, Synthetic construct
QSYDDSLDGHAV

SEQ ID NO: 116 - protein, 8 aa, Synthetic construct
GFTFSNYG

SEQ ID NO: 117 - protein, 8 aa, Synthetic construct
INSGGSTT

SEQ ID NO: 118 - protein, 13 aa, Synthetic construct
ARVDYSSSWLVDY

SEQ ID NO: 119 - protein, 9 aa, Synthetic construct
NTNIGSGYD

SEQ ID NO: 120 - protein, 12 aa, Synthetic construct
QSYDDNFDGHHV

SEQ ID NO: 121 - protein, 8 aa, Synthetic construct
GFTFSSYG

SEQ ID NO: 122 - protein, 8 aa, Synthetic construct
ISYGGGTY

SEQ ID NO: 123 - protein, 11 aa, Synthetic construct
SQWGDWGHFEY

SEQ ID NO: 124 - protein, 8 aa, Synthetic construct
RSNIGANY

SEQ ID NO: 125 - protein, 11 aa, Synthetic construct
SSWDDSLRGTV

SEQ ID NO: 126 - protein, 8 aa, Synthetic construct
GFTFSSDS

SEQ ID NO: 127 - protein, 13 aa, Synthetic construct
AKDSGFGSWYLES

SEQ ID NO: 128 - protein, 9 aa, Synthetic construct
KTNIGSGYD

SEQ ID NO: 129 - protein, 11 aa, Synthetic construct
QSFDDNVDGYV

SEQ ID NO: 130 - protein, 8 aa, Synthetic construct
GLTFNRNY

SEQ ID NO: 131 - protein, 8 aa, Synthetic construct
INPDGSTT

SEQ ID NO: 132 - protein, 10 aa, Synthetic construct
ARSSWGSFDY

SEQ ID NO: 133 - protein, 11 aa, Synthetic construct
QSFDDDLDGPV

SEQ ID NO: 134 - protein, 8 aa, Synthetic construct
GFTFSDYS

SEQ ID NO: 135 - protein, 8 aa, Synthetic construct
IMTSGSTY

SEQ ID NO: 136 - protein, 7 aa, Synthetic construct
HDYWFDF

SEQ ID NO: 137 - protein, 8 aa, Synthetic construct
GFTFSSYD

SEQ ID NO: 138 - protein, 8 aa, Synthetic construct
ISYDGSST

SEQ ID NO: 139 - protein, 15 aa, Synthetic construct
AKDSGSWSHFYGLDT

SEQ ID NO: 140 - protein, 6 aa, Synthetic construct
NIGRKS

SEQ ID NO: 141 - protein, 9 aa, Synthetic construct
QIWDRNINV

SEQ ID NO: 142 - protein, 8 aa, Synthetic construct
GFTFSRYY

SEQ ID NO: 143 - protein, 8 aa, Synthetic construct
ISSDGRRT

SEQ ID NO: 144 - protein, 10 aa, Synthetic construct
VREDYFDFDY

SEQ ID NO: 145 - protein, 8 aa, Synthetic construct
GFAFSGKT

SEQ ID NO: 146 - protein, 8 aa, Synthetic construct
IQHDGSRT

SEQ ID NO: 147 - protein, 10 aa, Synthetic construct
ADHPWYGPEY

SEQ ID NO: 148 - protein, 9 aa, Synthetic construct
HTNIGSGYD

SEQ ID NO: 149 - protein, 12 aa, Synthetic construct
QSYDDNLNDPYV

SEQ ID NO: 150 - protein, 8 aa, Synthetic construct
GFTFNKYH

SEQ ID NO: 151 - protein, 8 aa, Synthetic construct
ITSDGSAT

SEQ ID NO: 152 - protein, 14 aa, Synthetic construct
ARDQSSWGTGNFDY

SEQ ID NO: 153 - protein, 9 aa, Synthetic construct
DTNIGNHYD

SEQ ID NO: 154 - protein, 12 aa, Synthetic construct
QSYDDDLDGHIV

SEQ ID NO: 155 - protein, 8 aa, Synthetic construct
GFTLSSYW

SEQ ID NO: 156 - protein, 8 aa, Synthetic construct
ISGSGNTI

SEQ ID NO: 157 - protein, 10 aa, Synthetic construct
ATEYYWNIDN

SEQ ID NO: 158 - protein, 9 aa, Synthetic construct
SSNVGYGNY

SEQ ID NO: 159 - protein, 11 aa, Synthetic construct
SSYDSTLSGYV

SEQ ID NO: 160 - protein, 8 aa, Synthetic construct
INGGGSST

SEQ ID NO: 161 - protein, 12 aa, Synthetic construct
SNYYGIYWDSDY

SEQ ID NO: 162 - protein, 11 aa, Synthetic construct
QSFDDNVDGYV

SEQ ID NO: 163 - protein, 8 aa, Synthetic construct
GFTLSNYG

SEQ ID NO: 164 - protein, 8 aa, Synthetic construct
IRSGGNTY

SEQ ID NO: 165 - protein, 11 aa, Synthetic construct
STWDSSLNAYV

SEQ ID NO: 166 - protein, 8 aa, Synthetic construct
TNNIGIVG

SEQ ID NO: 167 - protein, 11 aa, Synthetic construct
QSVDATHGTIL

SEQ ID NO: 168 - protein, 8 aa, Synthetic construct
GFTFSSYA

SEQ ID NO: 169 - protein, 8 aa, Synthetic construct
INSAGSTT

SEQ ID NO: 170 - protein, 9 aa, Synthetic construct
GRYYYGLDY

SEQ ID NO: 171 - protein, 6 aa, Synthetic construct
SLSKYY

SEQ ID NO: 172 - protein, 10 aa, Synthetic construct
ESEVSPDTIV

SEQ ID NO: 173 - protein, 8 aa, Synthetic construct
GFTFSSFW

SEQ ID NO: 174 - protein, 8 aa, Synthetic construct
INSGGSIT

SEQ ID NO: 175 - protein, 11 aa, Synthetic construct
AKPDIYGSFDH

SEQ ID NO: 176 - protein, 10 aa, Synthetic construct
ESAVSSDTAV

SEQ ID NO: 177 - protein, 9 aa, Synthetic construct
GDSVTSSHF

SEQ ID NO: 178 - protein, 5 aa, Synthetic construct
WSDIT

SEQ ID NO: 179 - protein, 12 aa, Synthetic construct
ARYNGYVDYLDV

SEQ ID NO: 180 - protein, 9 aa, Synthetic construct
SSNVGYGDY

SEQ ID NO: 181 - protein, 11 aa, Synthetic construct
SSYDTSLNGFV

SEQ ID NO: 182 - protein, 9 aa, Synthetic construct
GGSVTTSDY

SEQ ID NO: 183 - protein, 5 aa, Synthetic construct
WKGRT

SEQ ID NO: 184 - protein, 10 aa, Synthetic construct
AHWGSGNLDY

SEQ ID NO: 185 - protein, 11 aa, Synthetic construct
QSLLHSNGNTY

SEQ ID NO: 186 - protein, 9 aa, Synthetic construct
GQGKQDPIT

SEQ ID NO: 187 - protein, 8 aa, Synthetic construct
GFTLSSNY

SEQ ID NO: 188 - protein, 8 aa, Synthetic construct
ISTDGSRT

SEQ ID NO: 189 - protein, 10 aa, Synthetic construct
AKNEYLAPTY

SEQ ID NO: 190 - protein, 11 aa, Synthetic construct
QSYDDNVDGYV

SEQ ID NO: 191 - protein, 8 aa, Synthetic construct
GFTFSSLS

SEQ ID NO: 192 - protein, 8 aa, Synthetic construct
ISPDGTIT

SEQ ID NO: 193 - protein, 13 aa, Synthetic construct
AKSRYTDTYPFDY

SEQ ID NO: 194 - protein, 9 aa, Synthetic construct
GQGIQYPPT

SEQ ID NO: 195 - protein, 8 aa, Synthetic construct
GFTFSSYW

SEQ ID NO: 196 - protein, 8 aa, Synthetic construct
ISDSGSST

SEQ ID NO: 197 - protein, 8 aa, Synthetic construct
ARGLGMDY

SEQ ID NO: 198 - protein, 10 aa, Synthetic construct
ESLVDSDAYM

SEQ ID NO: 199 - protein, 11 aa, Synthetic construct
HSYDENLDGYV

SEQ ID NO: 200 - protein, 6 aa, Synthetic construct
SLNKYY

SEQ ID NO: 201 - protein, 10 aa, Synthetic construct
ESLVNDNTYV

SEQ ID NO: 202 - protein, 10 aa, Synthetic construct
ESVDSTDTYV

SEQ ID NO: 203 - protein, 6 aa, Synthetic construct
SLTKYY

SEQ ID NO: 204 - protein, 10 aa, Synthetic construct
ESMDGREAYV

SEQ ID NO: 205 - protein, 11 aa, Synthetic construct
QSYDDNLDGYV

SEQ ID NO: 206 - protein, 8 aa, Synthetic construct
GFTFSHYD

SEQ ID NO: 207 - protein, 8 aa, Synthetic construct
IAYDGSNT

SEQ ID NO: 208 - protein, 6 aa, Synthetic construct
ARGLVY

SEQ ID NO: 209 - protein, 11 aa, Synthetic construct
QSLLHSDGNTY

SEQ ID NO: 210 - protein, 9 aa, Synthetic construct
GQGTHLPFT

SEQ ID NO: 211 - protein, 8 aa, Synthetic construct
GLTFSDNY

SEQ ID NO: 212 - protein, 8 aa, Synthetic construct
ARWWDFEY

SEQ ID NO: 213 - protein, 6 aa, Synthetic construct
SLSEYY

SEQ ID NO: 214 - protein, 10 aa, Synthetic construct
ESAVNSDGIV

SEQ ID NO: 215 - protein, 8 aa, Synthetic construct
ISDTGSTY

SEQ ID NO: 216 - protein, 10 aa, Synthetic construct
KDLGVWGMDN

SEQ ID NO: 217 - protein, 11 aa, Synthetic construct
QSFDTTLDAYV

SEQ ID NO: 218 - protein, 8 aa, Synthetic construct
GFTLRDYS

SEQ ID NO: 219 - protein, 8 aa, Synthetic construct
ISTGGSTT

SEQ ID NO: 220 - protein, 7 aa, Synthetic construct
TTTALGY

SEQ ID NO: 221 - protein, 6 aa, Synthetic construct
SLNDYY

SEQ ID NO: 222 - protein, 10 aa, Synthetic construct
ESEVSAGGAV

SEQ ID NO: 223 - protein, 8 aa, Synthetic construct
GFNFGNYA

SEQ ID NO: 224 - protein, 8 aa, Synthetic construct
ISNDGTIT

SEQ ID NO: 225 - protein, 8 aa, Synthetic construct
VAGGFLDY

SEQ ID NO: 226 - protein, 10 aa, Synthetic construct
ASADSSAIPV

SEQ ID NO: 227 - protein, 8 aa, Synthetic construct
ITYDGSNT

SEQ ID NO: 228 - protein, 13 aa, Synthetic construct
VASPLYGTYGFDY

SEQ ID NO: 229 - protein, 8 aa, Synthetic construct
TKNIGIVG

SEQ ID NO: 230 - protein, 11 aa, Synthetic construct
QSFDTTLDAHV

SEQ ID NO: 231 - protein, 9 aa, Synthetic construct
GGSVIRGYY

SEQ ID NO: 232 - protein, 5 aa, Synthetic construct
WAGST

SEQ ID NO: 233 - protein, 12 aa, Synthetic construct
VGSLRGGWLFDN

SEQ ID NO: 234 - protein, 8 aa, Synthetic construct
SSSVGTHY

SEQ ID NO: 235 - protein, 11 aa, Synthetic construct
SSWDDSLGAYV

SEQ ID NO: 236 - protein, 8 aa, Synthetic construct
GFTFSSSV

SEQ ID NO: 237 - protein, 8 aa, Synthetic construct
INGGGSFA

SEQ ID NO: 238 - protein, 8 aa, Synthetic construct
VGGGYMDF

SEQ ID NO: 239 - protein, 10 aa, Synthetic construct
ESAVSTETVV

SEQ ID NO: 240 - protein, 8 aa, Synthetic construct
GFTFSGYD

SEQ ID NO: 241 - protein, 8 aa, Synthetic construct
VKGGWLDF

SEQ ID NO: 242 - protein, 8 aa, Synthetic construct
TNDIGIIG

SEQ ID NO: 243 - protein, 11 aa, Synthetic construct
QSVDPALHTYV

SEQ ID NO: 244 - protein, 8 aa, Synthetic construct
GFTFTDYD

SEQ ID NO: 245 - protein, 7 aa, Synthetic construct
VVSNMNL

SEQ ID NO: 246 - protein, 11 aa, Synthetic construct
QSLLHSDGKSY

SEQ ID NO: 247 - protein, 9 aa, Synthetic construct
QQGLHFPPT

SEQ ID NO: 248-259 - skipped (12 entries)

SEQ ID NO: 260 - protein, 11 aa, Synthetic construct
SGGGGSGGGGS

SEQ ID NO: 261 - skipped

SEQ ID NO: 262 - DNA, 369 bp, Synthetic construct
  source 1..369: mol_type = other DNA
atggccttaccagtgaccgccttgctcctgccgctggccttgctgctccacgccgccaggccgggatccgctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 263 - protein, 122 aa, Synthetic construct
MALPVTALLLPLALLLHAARPGSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 264 - DNA, 30 bp, Synthetic construct
  source 1..30: mol_type = other DNA
gagcagaaattaatctcagaggaggacctg

SEQ ID NO: 265 - DNA, 222 bp, Synthetic construct
  source 1..222: mol_type = other DNA
cccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgca

SEQ ID NO: 266 - protein, 75 aa, Synthetic construct
SPTEPSSKTGNPRHLHVLIGTSVVKIPFTILLFFLLHRWCSNKKNAAVMDQEPAGNRTVNSEDSDEQDHQEVSYA

SEQ ID NO: 267-270 - skipped (4 entries)

SEQ ID NO: 271 - protein, 109 aa, Synthetic construct
QAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVL

SEQ ID NO: 272 - protein, 110 aa, Synthetic construct
SYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTIL

SEQ ID NO: 273 - protein, 111 aa, Synthetic construct
QSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVL

SEQ ID NO: 274 - protein, 110 aa, Synthetic construct
QSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVL

SEQ ID NO: 275 - protein, 110 aa, Synthetic construct
QPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVL

SEQ ID NO: 276 - protein, 112 aa, Synthetic construct
QPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTIL

SEQ ID NO: 277 - protein, 112 aa, Synthetic construct
SYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVL

SEQ ID NO: 278 - protein, 110 aa, Synthetic construct
QSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVL

SEQ ID NO: 279 - protein, 111 aa, Synthetic construct
QPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTIL

SEQ ID NO: 280 - protein, 111 aa, Synthetic construct
QAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVL

SEQ ID NO: 281 - protein, 110 aa, Synthetic construct
QPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVL

SEQ ID NO: 282 - protein, 106 aa, Synthetic construct
SYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVL

SEQ ID NO: 283 - protein, 110 aa, Synthetic construct; identical to SEQ ID NO: 281

SEQ ID NO: 284 - protein, 112 aa, Synthetic construct
QSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVL

SEQ ID NO: 285 - protein, 112 aa, Synthetic construct
SYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVL

SEQ ID NO: 286 - protein, 111 aa, Synthetic construct
QSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTIL

SEQ ID NO: 287 - protein, 111 aa, Synthetic construct
QPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTIL

SEQ ID NO: 288 - protein, 110 aa, Synthetic construct
QSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVL

SEQ ID NO: 289 - protein, 110 aa, Synthetic construct
QAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVL

SEQ ID NO: 290 - protein, 107 aa, Synthetic construct
QSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVL

SEQ ID NO: 291 - protein, 107 aa, Synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVL

SEQ ID NO: 292 - protein, 111 aa, Synthetic construct
QPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTIL

SEQ ID NO: 293 - protein, 112 aa, Synthetic construct
DIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIK

SEQ ID NO: 294 - protein, 111 aa, Synthetic construct
QSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVL

SEQ ID NO: 295 - protein, 112 aa, Synthetic construct
DIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELK

SEQ ID NO: 296 - protein, 107 aa, Synthetic construct
SSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVL

SEQ ID NO: 297 - protein, 108 aa, Synthetic construct
QPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVL

SEQ ID NO: 298 - protein, 107 aa, Synthetic construct
QSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVL

SEQ ID NO: 299 - protein, 107 aa, Synthetic construct
QSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTIL

SEQ ID NO: 300 - protein, 108 aa, Synthetic construct
QSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVL

SEQ ID NO: 301 - protein, 107 aa, Synthetic construct
QSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVL

SEQ ID NO: 302 - protein, 108 aa, Synthetic construct
QSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVL

SEQ ID NO: 303 - protein, 112 aa, Synthetic construct
DIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIK

SEQ ID NO: 304 - protein, 112 aa, Synthetic construct
SYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTIL

SEQ ID NO: 305 - protein, 107 aa, Synthetic construct
SSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVL

SEQ ID NO: 306 - protein, 110 aa, Synthetic construct
QSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVL

SEQ ID NO: 307 - protein, 107 aa, Synthetic construct
SSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVL

SEQ ID NO: 308 - protein, 107 aa, Synthetic construct
SSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVL

SEQ ID NO: 309 - protein, 110 aa, Synthetic construct
QSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVL

SEQ ID NO: 310 - protein, 110 aa, Synthetic construct
QSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTIL

SEQ ID NO: 311 - protein, 107 aa, Synthetic construct
QPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVL

SEQ ID NO: 312 - protein, 110 aa, Synthetic construct
QSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTIL

SEQ ID NO: 313 - protein, 112 aa, Synthetic construct
DIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELK

SEQ ID NO: 314 - protein, 122 aa, Synthetic construct
EVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSS

SEQ ID NO: 315 - protein, 121 aa, Synthetic construct
EEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSS

SEQ ID NO: 316 - protein, 117 aa, Synthetic construct
EVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSS

SEQ ID NO: 317 - protein, 119 aa, Synthetic construct
EEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSS

SEQ ID NO: 318 - protein, 117 aa, Synthetic construct
EVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSS

SEQ ID NO: 319 - protein, 118 aa, Synthetic construct
EVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSS

SEQ ID NO: 320 - protein, 120 aa, Synthetic construct
EVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSS

SEQ ID NO: 321 - protein, 118 aa, Synthetic construct
EVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSS

SEQ ID NO: 322 - protein, 120 aa, Synthetic construct
EVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSP

SEQ ID NO: 323 - protein, 114 aa, Synthetic construct
EVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSS

SEQ ID NO: 324 - protein, 122 aa, Synthetic construct
EEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSS

SEQ ID NO: 325 - protein, 117 aa, Synthetic construct
EVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSS

SEQ ID NO: 326 - protein, 117 aa, Synthetic construct
EVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSS

SEQ ID NO: 327 - protein, 121 aa, Synthetic construct
EVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSS

SEQ ID NO: 328 - protein, 117 aa, Synthetic construct
EVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSS

SEQ ID NO: 329 - protein, 119 aa, Synthetic construct
EVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSS

SEQ ID NO: 330 - protein, 118 aa, Synthetic construct
EVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSS

SEQ ID NO: 331 - protein, 117 aa, Synthetic construct
EVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSS

SEQ ID NO: 332 - protein, 116 aa, Synthetic construct
EVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSS

SEQ ID NO: 333 - protein, 118 aa, Synthetic construct
EVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSS

SEQ ID NO: 334 - protein, 117 aa, Synthetic construct
ELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSS

SEQ ID NO: 335 - protein, 115 aa, Synthetic construct
ELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSS

SEQ ID NO: 336 - protein, 117 aa, Synthetic construct
  SITE 10: note = misc_feature - Xaa can be any naturally occurring amino acid
EVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSS

SEQ ID NO: 337 - protein, 120 aa, Synthetic construct
EEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSS

SEQ ID NO: 338 - protein, 115 aa, Synthetic construct
EVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 339 - protein, 115 aa, Synthetic construct
EVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 340 - protein, 115 aa, Synthetic construct; identical to SEQ ID NO: 338

SEQ ID NO: 341 - protein, 115 aa, Synthetic construct
EVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 342 - protein, 115 aa, Synthetic construct; identical to SEQ ID NO: 339

SEQ ID NO: 343 - protein, 115 aa, Synthetic construct
EVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSS

SEQ ID NO: 344 - protein, 115 aa, Synthetic construct; identical to SEQ ID NO: 339

SEQ ID NO: 345 - protein, 113 aa, Synthetic construct
EVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSS

SEQ ID NO: 346 - protein, 118 aa, Synthetic construct
EVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSS

SEQ ID NO: 347 - protein, 115 aa, Synthetic construct
EVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSS

SEQ ID NO: 348 - protein, 117 aa, Synthetic construct
EEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSS

SEQ ID NO: 349 - protein, 114 aa, Synthetic construct
EVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSS

SEQ ID NO: 350 - protein, 115 aa, Synthetic construct
EVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSS

SEQ ID NO: 351 - protein, 120 aa, Synthetic construct
EVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSS

SEQ ID NO: 352 - protein, 117 aa, Synthetic construct
ELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSS

SEQ ID NO: 353 - protein, 115 aa, Synthetic construct
EVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSS

SEQ ID NO: 354 - protein, 115 aa, Synthetic construct
EVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSS

SEQ ID NO: 355 - protein, 114 aa, Synthetic construct
EVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSS

SEQ ID NO: 356 - protein, 21 aa, Synthetic construct
MALPVTALLLPLALLLHAARP

SEQ ID NO: 357-368 - skipped (12 entries)

SEQ ID NO: 369 - protein, 372 aa, Synthetic construct
MALPVTALLLPLALLLHAARPGSQAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVLGGGSSRSSSSGGGGSGGGGEVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 370 - protein, 372 aa, Synthetic construct
MALPVTALLLPLALLLHAARPGSSYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTILGGGSSRSSSSGGGGSGGGGEEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 371 - protein, 369 aa, Synthetic construct
MALPVTALLLPLALLLHAARPGSQSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVLGGGSSRSSSSGGGGSGGGGEVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 372 - protein, 370 aa, Synthetic construct
MALPVTALLLPLALLLHAARPGSQSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVLGGGSSRSSSSGGGGSGGGGEEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 373 - protein, 368 aa, Synthetic construct
MALPVTALLLPLALLLHAARPGSQPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVLGGGSSRSSSSGGGGSGGGGEVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 374 - protein, 371 aa, Synthetic construct
MALPVTALLLPLALLLHAARPGSQPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 375 - protein, 373 aa, Synthetic construct
MALPVTALLLPLALLLHAARPGSSYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVLGGGSSRSSSSGGGGSGGGGEVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 376 - protein, 369 aa, Synthetic construct
MALPVTALLLPLALLLHAARPGSQSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVLGGGSSRSSSSGDGGSGGGGEVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 377 - protein, 372 aa, Synthetic construct
MALPVTALLLPLALLLHAARPGSQPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTILGGGSSRSSSSGGGGSGGGGEVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSPASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 378 - protein, 369 aa, Synthetic construct
MALPVTALLLPLALLLHAARPGSQAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVLGGGSSRSSSSGGGGSGGGGEVQLVQSGGDLVKPGGSLKISCVASGLTFNRNYMTWIRQAPGKGLQWVSEINPDGSTTSYTDAVRGRFTISRDNAKNTLYLQMNSMRDEDTAVYYCARSSWGSFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 379 - protein, 365 aa, Synthetic construct
MALPVTALLLPLALLLHAARPGSQPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGGGGSGGGGEVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 380 - protein, 369 aa, Synthetic construct
MALPVTALLLPLALLLHAARPGSSYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVLGGGSSRSSSSGGGGSGGGGEEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 381 - protein, 368 aa, Synthetic construct
MALPVTALLLPLALLLHAARPGSQPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGDGGSGGGGEVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 382 - protein, 370 aa, Synthetic construct
MALPVTALLLPLALLLHAARPGSQSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVLGGGSSRSSSSGGGGSGGGGEVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 383 - protein, 374 aa, Synthetic construct
MALPVTALLLPLALLLHAARPGSSYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVLGGGSSRSSSSGGGGSGGGGEVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 384 - protein, 369 aa, Synthetic construct
MALPVTALLLPLALLLHAARPGSQSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTILGGGSSRSSSSGGGGSGGGGEVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 385 - protein, 371 aa, Synthetic construct
MALPVTALLLPLALLLHAARPGSQPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTILGGGSSRSSSSGGGGSGGGGEVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 386 - protein, 369 aa, Synthetic construct
MALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 387 - protein, 368 aa, Synthetic construct
MALPVTALLLPLALLLHAARPGSQAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVLGGGSSRSSSSGGGGSGGGGEVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 388 - protein, 364 aa, Synthetic construct
MALPVTALLLPLALLLHAARPGSQSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVLGGGSSRSSSSGGGGSGGGGEVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 389 - protein, 366 aa, Synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVLGGGSSRSSSSGGGGSGGGGEVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 390 - protein, 369 aa, Synthetic construct
MALPVTALLLPLALLLHAARPGSQPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTILGGGSSRSSSSGGGGSGGGGELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 391 - protein, 367 aa, Synthetic construct
MALPVTALLLPLALLLHAARPGSDIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIKGGSSRSSSSGGGGSGGGGELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 392 - protein, 369 aa, Synthetic construct
  SITE 163: note = misc_feature - Xaa can be any naturally occurring amino acid
MALPVTALLLPLALLLHAARPGSQSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVLGGGSSRSSSSGGGGSGGGGEVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 393 - protein, 372 aa, Synthetic construct
MALPVTALLLPLALLLHAARPGSDIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELKGGSSRSSSSGGGGSGGGGEEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 394 - protein, 363 aa, Synthetic construct
MALPVTALLLPLALLLHAARPGSSSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 395 - protein, 364 aa, Synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 396 - protein, 363 aa, Synthetic construct
MALPVTALLLPLALLLHAARPGSQSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 397 - protein, 363 aa, Synthetic construct
MALPVTALLLPLALLLHAARPGSQSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTILGGGSSRSSSSGGGGSGGGGEVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 398 - protein, 364 aa, Synthetic construct
MALPVTALLLPLALLLHAARPGSQSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 399 - protein, 363 aa, Synthetic construct
MALPVTALLLPLALLLHAARPGSQSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVLGGGSSRSSSSGGGGSGGGGEVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 400 - protein, 364 aa, Synthetic construct
MALPVTALLLPLALLLHAARPGSQSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 401 - protein, 365 aa, Synthetic construct
MALPVTALLLPLALLLHAARPGSDIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIKGGSSRSSSSGGGGSGGGGEVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 402 - protein, 371 aa, Synthetic construct
MALPVTALLLPLALLLHAARPGSSYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 403 - protein, 363 aa, Synthetic construct
MALPVTALLLPLALLLHAARPGSSSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVLGGGSSRSSSSGGGGSGGGGEVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 404 - protein, 368 aa, Synthetic construct
MALPVTALLLPLALLLHAARPGSQSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVLGGGSSRSSSSGGGGSGGGGEEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 405 - protein, 362 aa, Synthetic construct
MALPVTALLLPLALLLHAARPGSSSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVLGGGSSRSSSSGGGGSGGGGEVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 406 - protein, 363 aa, Synthetic construct
MALPVTALLLPLALLLHAARPGSSSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVLGGGSSRSSSSGGGGSGGGGEVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 407 - protein, 371 aa, Synthetic construct
MALPVTALLLPLALLLHAARPGSQSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVLGGGSSRSSSSGGGGSGGGGEVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 408 - protein, 368 aa, Synthetic construct
MALPVTALLLPLALLLHAARPGSQSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTILGGGSSRSSSSGGGGSGGGGELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 409 - protein, 363 aa, Synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVLGGGSSRSSSSGGGGSGGGGEVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 410 - protein, 366 aa, Synthetic construct
MALPVTALLLPLALLLHAARPGSQSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTILGGGSSRSSSSGGGGSGGGGEVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 411 - protein, 366 aa, Synthetic construct
MALPVTALLLPLALLLHAARPGSDIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELKGGSSRSSSSGGGGSGGGGEVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 412 - protein, 19 aa, Synthetic construct
GGGSSRSSSSGGGGSGGGG

SEQ ID NO: 413 - DNA, 861 bp, Synthetic construct
  source 1..861: mol_type = other DNA
atggacttccaggtgcagatcatcagcttcctgctgatcagcgccagcgtgatcatgagccggggccagatcgtgctgagccagagccccgccatcctgagcgccagccccggcgagaaggtgaccatgacctgccgggccagcagcagcgtgagctacatccactggttccagcagaagcccggcagcagccccaagccctggatctacgccaccagcaacctggccagcggcgtgcccgtgcggttcagcggcagcggcagcggcaccagctacagcctgaccatcagccgggtggaggccgaggacgccgccacctactactgccagcagtggaccagcaacccccccaccttcggcggcggcaccaagctggagatcaagggcggcggcagcagccggagcagcagcagcggcggcggcggcagcggcggcggcggcatgggctggagcctgatcctgctgttcctggtggccgtggccacccgggtgctgagccaggtgcagctgcagcagcccggcgccgagctggtgaagcccggcgccagcgtgaagatgagctgcaaggccagcggctacaccttcaccagctacaacatgcactgggtgaagcagacccccggccggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgcccggagcacctactacggcggcgactggtacttcaacgtgtggggcgccggcaccaccgtgaccgtgagcgcc

SEQ ID NO: 414 - DNA, 685 bp, Synthetic construct
  source 1..685: mol_type = other DNA
gagatcgtgctgacccagagccccgccaccctgagcctgagccccggcgagcgggccaccctgagctgccgggccagccagagcgtgagcagctacctggcctggtaccagcagaagcccggccaggccccccggctgctgatctacgacgccagcaaccgggccaccggcatccccgcccggttcagcggcagcggcagcggcaccgacttcaccctgaccatcagcagcctggagcccgaggacttcgccgtgtactactgccagcagcggagcaactggcccatcaccttcggccagggcacccggctggagatcaagggcggcggcagcagccggagcagcagcagcggcggcggcggcagcggcggcggcggcgaggtgcagctggtggagagcggcggcggcctggtgcagcccggccggagcctgcggctgagctgcgccgccagcggcttcaccttcaacgactacgccatgcactgggtgcggcaggcccccggcaagggcctggagtgggtgagcaccatcagctggaacagcggcagcatcggctacgccgacagcgtgaagggccggttcaccatcagccgggacaacgccaagaagagcctgtacctgcagatgaacagcctgcgggccgaggacaccgccctgtactactgcgccaaggacatccagtacg

SEQ ID NO: 415 - DNA, 744 bp, Synthetic construct
  source 1..744: mol_type = other DNA
gagatcgtgctgacccagagccccgccaccctgagcctgagccccggcgagcgggccaccctgagctgccgggccagccagagcgtgagcagctacctggcctggtaccagcagaagcccggccaggccccccggctgctgatctacgacgccagcaaccgggccaccggcatccccgcccggttcagcggcagcggcagcggcaccgacttcaccctgaccatcagcagcctggagcccgaggacttcgccgtgtactactgccagcagcggagcaactggcccatcaccttcggccagggcacccggctggagatcaagggcggcggcagcagccggagcagcagcagcggcggcggcggcagcggcggcggcggcgaggtgcagctggtggagagcggcggcggcctggtgcagcccgaccggagcctgcggctgagctgcgccgccagcggcttcaccttccacgactacgccatgcactgggtgcggcaggcccccggcaagggcctggagtgggtgagcaccatcagctggaacagcggcaccatcggctacgccgacagcgtgaagggccggttcaccatcagccgggacaacgccaagaacagcctgtacctgcagatgaacagcctgcgggccgaggacaccgccctgtactactgcgccaaggacatccagtacggcaactactactacggcatggacgtgtggggccagggcaccaccgtgaccgtgagcagc

SEQ ID NO: 416 - DNA, 753 bp, Synthetic construct
  source 1..753: mol_type = other DNA
gagatcgtgctgacccagagccccgccaccctgagcctgagccccggcgagcgggccaccctgagctgccgggccagccagagcgtgagcagctacctggcctggtaccagcagaagcccggccaggccccccggctgctgatctacgacgccagcaaccgggccaccggcatccccgcccggttcagcggcagcggcagcggcaccgacttcaccctgaccatcagcagcctggagcccgaggacttcgccgtgtactactgccagcagcggagcgactggcccctgaccttcggcggcggcaccaaggtggagatcaagggcggcggcagcagccggagcagcagcagcggcggcggcggcagcggcggcggcggcgaggtgcagctggtgcagagcggcggcggcctggtgcaccccggcggcagcctgcggctgagctgcaccggcagcggcttcaccttcagctaccacgccatgcactgggtgcggcaggcccccggcaagggcctggagtgggtgagcatcatcggcaccggcggcgtgacctactacgccgacagcgtgaagggccggttcaccatcagccgggacaacgtgaagaacagcctgtacctgcagatgaacagcctgcgggccgaggacatggccgtgtactactgcgcccgggactactacggcgccggcagcttctacgacggcctgtacggcatggacgtgtggggccagggcaccaccgtgaccgtgagcagc

SEQ ID NO: 417 - DNA, 2058 bp, Synthetic construct
  source 1..2058: mol_type = other DNA
gacatcgtgatgacccagacccccctgagcctgcccgtgacccccggcgagcccgccagcatcagctgccggagcagcaagagcctgctgcacagcaacggcatcacctacctgtactggtacctgcagaagcccggccagagcccccagctgctgatctaccagatgagcaacctggtgagcggcgtgcccgaccggttcagcggcagcggcagcggcaccgacttcaccctgaagatcagccgggtggaggccgaggacgtgggcgtgtactactgcgcccagaacctggagctgccctacaccttcggcggcggcaccaaggtggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgcggcggcggcagcagccggagcagcagcagcggcggcggcggcagcggcggcggcggccaggtgcagctggtgcagagcggcgccgaggtgaagaagcccggcagcagcgtgaaggtgagctgcaaggccagcggctacgccttcagctacagctggatcaactgggtgcggcaggcccccggccagggcctggagtggatgggccggatcttccccggcgacggcgacaccgactacaacggcaagttcaagggccgggtgaccatcaccgccgacaagagcaccagcaccgcctacatggagctgagcagcctgcggagcgaggacaccgccgtgtactactgcgcccggaacgtgttcgacggctactggctggtgtactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggc

SEQ ID NO: 418 - DNA, 738 bp, Synthetic construct
  source 1..738: mol_type = other DNA
cagatcgtgctgagccagagccccgccatcctgagcgccagccccggcgagaaggtgaccatgacctgccgggccagcagcagcgtgagctacatccactggttccagcagaagcccggcagcagccccaagccctggatctacgccaccagcaacctggccagcggcgtgcccgtgcggttcagcggcagcggcagcggcaccagctacagcctgaccatcagccgggtggaggccgaggacgccgccacctactactgccagcagtggaccagcaacccccccaccttcggcggcggcaccaagctggagatcaagggcggcggcagcagccggagcagcagcagcggcggcggcggcagcggcggcggcggccaggtgcagctgcagcagcccggcgccgagctggtgaaggccggcgccagcgtgaagatgagctgcaaggccagcggctacaccttcaccagctacaacatgcactgggtgaagcagacccccggccggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgcccggagcacctactacggcggcgacacctacttcaacgtgtggggcgccggcaccaccgtgaccgtgagcgcc

SEQ ID NO: 419 - DNA, 738 bp, Synthetic construct
  source 1..738: mol_type = other DNA
cagatcgtgctgagccagagccccgccatcatcaccgccagccccggcgagaaggtgaccatgacctgccgggccagcaccagcgccagctacatccactggttccagcagaagcccaccagcagccccaagccctggatctacgccaccagcaacctggccagcggcgtgcccagccggttcagcggcagcggcagcggcaccacctacagcatgaccatcagcagcctggaggccgaggacgccgccacctactactgccagcagtggaccagcaacccccccaccttcggcggcggcaccaagctggagatcaagggcggcggcagcagccggagcagcagcagcggcggcggcggcagcggcggcggcggccaggtgcagctgcagcagcccggcgccgagctgaagaagcccggcgccagcgtgaaggtgagctgcaaggccagcggctacaccttcaccagctacaacatgcactgggtgaagcagacccccggccggggcctggagtggaccggcgccatctaccccggcaacggcgacaccagctacaaccagaagttcaagggcaagaccaccctgaccgccgacaagagcagcagcaccgcctacatggagctgagcagcctgcggagcgaggacaccgccgtgtactactgcgcccggagcacctactacggcggcgactggtacttcaacgtgtggggcgccggcaccaccgtgaccgtgagcgcc

SEQ ID NO: 420 - DNA, 738 bp, Synthetic construct
  source 1..738: mol_type = other DNA
cagatcgtgctgagccagagccccgccatcatcaccgccagccccggcgagaaggtgaccatgacctgccgggccagcaccagcgtgagctacatccactggttccagcagaagcccaccagcagccccaagccctggatctacgccaccagcaacctggccagcggcgtgcccagccggttcagcggcagcggcagcggcaccacctacagcatgaccatcagcagcctggaggccgaggacgccgccacctactactgccagcagtggaccagcaacccccccaccttcggcggcggcaccaagctggagatcaagggcggcggcagcagccggagcagcagcagcggcggcggcggcagcggcggcggcggccaggtgcagctgcagcagcccggcgccgagctgaagaagcccggcgccagcgtgaaggtgagctgcaaggccagcggctacaccttcaccagctacaacatgcactgggtgaagcagacccccggccggggcctggagtggatcggcgccatctaccccggcaacggcgacaccagctacaaccagaagttcaagggcaagaccaccctgaccgccgacaagagcagcagcaccgcctacatggagctgagcagcctgcggagcgaggacaccgccgtgtactactgcgcccggagcacctactacggcggcgactggtacttcaacgtgtggggcgccggcaccaccgtgaccgtgagcgcc

SEQ ID NO: 421 - DNA, 741 bp, Synthetic construct
  source 1..741: mol_type = other DNA
gacatcgtgctgacccagagccccgccatcctgagcgccagccccggcgagaaggtgaccatgacctgccgggccagcagcagcgtgaactacatggactggtaccagaagaagcccggcagcagccccaagccctggatctacgccaccagcaacctggccagcggcgtgcccgcccggttcagcggcagcggcagcggcaccagctacagcctgaccatcagccgggtggaggccgaggacgccgccacctactactgccagcagtggagcttcaacccccccaccttcggcggcggcaccaagctggagatcaagggcggcggcagcagccggagcagcagcagcggcggcggcggcagcggcggcggcggcgaggtgcagctgcagcagagcggcgccgagctggtgaagcccggcgccagcgtgaagatgagctgcaaggccagcggctacaccttcaccagctacaacatgcactgggtgaagcagacccccggccagggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgactactactgcgcccggagcaactactacggcagcagctactggttcttcgacgtgtggggcgccggcaccaccgtgaccgtgagcagc

SEQ ID NO: 422 - DNA, 741 bp, Synthetic construct
  source 1..741: mol_type = other DNA
gacatcgtgctgacccagagccccgccatcatcaccgccagccccggcgagaaggtgaccatgacctgccgggccagcagcagcgtgaactacatggactggtaccagaagaagcccggcagcagccccaagccctggatctacgccaccagcaacctggccagcggcgtgcccagccggttcagcggcagcggcagcggcaccacctacagcatgaccatcagcagcctggaggccgaggacgccgccacctactactgccagcagtggagcttcaacccccccaccttcggcggcggcaccaagctggagatcaagggcggcggcagcagccggagcagcagcagcggcggcggcggcagcggcggcggcggcgaggtgcagctgcagcagagcggcgccgagctgaagaagcccggcgccagcgtgaaggtgagctgcaaggccagcggctacaccttcaccagctacaacatgcactgggtgaagcagacccccggccagggcctggagtggatcggcgccatctaccccggcaacggcgacaccagctacaaccagaagttcaagggcaagaccaccctgaccgccgacaagagcagcagcaccgcctacatggagctgagcagcctgcggagcgaggacaccgccgtgtactactgcgcccggagcaactactacggcagcagctactggttcttcgacgtgtggggcaccggcaccaccgtgaccgtgagcagc

SEQ ID NO: 423 - DNA, 741 bp, Synthetic construct
  source 1..741: mol_type = other DNA
cagatcgtgctgagccagagccccaccatcctgagcgccagccccggcgagaaggtgaccatgacctgccgggccagcagcagcgtgagctacatggactggtaccagcagaagcccggcagcagccccaagccctggatctacgccaccagcaacctggccagcggcgtgcccgcccggttcagcggcagcggcagcggcaccagctacagcctgaccatcagccgggtggaggccgaggacgccgccacctactactgccagcagtggatcagcaacccccccaccttcggcgccggcaccaagctggagctgggcggcggcagcagccggagcagcagcagcggcggcggcggcagcggcggcggcggccaggtgcagctgcagcagcccggcgccgagctggtgaagcccggcgccagcgtgaagatgagctgcaagaccagcggctacaccttcaccagctacaacgtgcactgggtgaagcagacccccggccagggcctggagtggatcggcgccatctaccccggcaacggcgacaccagcttcaaccagaagttcaagggcaaggccaccctgaccgccgacaagagcagcagcaccgtgtacatgcagctgagcagcctgaccagcgaggacagcgccgtgtactactgcgcccggagcaactactacggcagcagctacgtgtggttcttcgacgtgtggggcgccggcaccaccggcaccggcagcagc

SEQ ID NO: 424 - DNA, 759 bp, Synthetic construct
  source 1..759: mol_type = other DNA
gacatcgtgatgacccagacccccctgagcagccccgtgaccctgggccagcccgccagcatcagctgccggagcagccagagcctggtgtacagcgacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagcacccggcaggtgttcgagcccgccctggagaccgacttcgtgggcggcggcgtgggctacacctacaagtgcgtggtggagcagatcgccaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtaccggcccagcttccagggccaggtgaccatcagcgccgacaagagcatcaccaccgcctacctgcagtggagcagcctgaaggccagcgacaccgccatgtactactgcgcccggcaccccagctacggcagcggcagccccaacttcgactactggggccagggcaccctggtgaccgtgagcagc

SEQ ID NO: 425 - DNA, 690 bp, Synthetic construct
  source 1..690: mol_type = other DNA
gagatcgtgatgacccagagccccgccaccctgagcgtgagccccggcgagcgggccaccctgagctgccgggccagccagagcgtgagcagcaacctggcctggtaccagcagaagcccggccaggccccccggctgctgatcagcggcgccagcacccgggccaccggcatccccgcccggttcagcggcagcggcagcggcaccgagttcaccctgaccatcagcagcctgcagagcgaggacttcgccgtgtactactgccaccagtacaacgactggagcctgaccttcggcggcggcaccaaggtggagatcaagggcggcggcagcagccggagcagcagcagcggcggcggcggcagcggcggcggcggcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggatgggcgacttctggagcggctactacacccggggcatggacgtgtggggccagggcaccaccgtgaccgtgagcagc

SEQ ID NO: 426 - DNA, 756 bp, Synthetic construct
  source 1..756: mol_type = other DNA
gacatcgtgatgacccagacccccctgagcagccccgtgaccctgggccagcccgccagcatcagctgccggagcagccagcggctggtgcacagcgacggccacacctacctgagctggctgcagcagcggcccggccagcccccccggctgctgatcagcaaggtgagcaaccggttcagcggcgtgcccgaccggttcagcggcagcggcgccggcaccgacttcaccctgaagatcagccgggtggaggccgaggacgtgggcatctactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccaactactggatcgtgtgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcacggcgactactactactactacggcatggacgtgtggggccagggcaccaccgtgaccgtgagcagc

SEQ ID NO: 427 - DNA, 759 bp, Synthetic construct
  source 1..759: mol_type = other DNA
gacatcgtgatgacccagacccccctgagcagccccgtgaccctgggccagcccgccagcatcagctgccggagcagccagagcctggtgtacagcgacggcaacacctacctgagctggctgcagcagcggcccggccagcccccccggctgctgttctacaagatcagcaaccggttcagcggcgtgcccgaccggttcagcggcagcggcgccggcaccgacttcaccctgaagatcagccgggtggaggccgaggacgtgggcgtgtactactgcgtgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgagcggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatctactactgcgcccggcaccccagctacggcagcggcagccccaacttcgactactggggccagggcaccctggtgaccgtgagcagc

SEQ ID NO: 428 - DNA, 756 bp, Synthetic construct
  source 1..756: mol_type = other DNA
gacgtggtgatgacccagagccccctgagcctgagcgtgaccctgggccagcccgccagcatcagctgccggagcagccagagcctggtgtacaacgacggcgacacctacctgaactggttccagcagcggcccggccagagcccccggctgctgatctacaaggtgagcaactgggacagcggcgtgcccgaccggttcagcggcagcggcagcggcaccgacttcaccctgaaggtgagccgggtggaggccgaggacgtgggcgtgtactactgcatgcagggcacccactggcccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccaactactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgactactaccactacagcggcatggacgtgtggggccagggcaccaccgtgaccgtgagcagc

SEQ ID NO: 429 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggcaagacctacctgagctggctgcagcagcggcccggccagcccccccggctgctgatctacaagagcagcaaccggttcagcggcgtgcccgaccggttcagcggcagcggcgccggcaccgacttcaccctgaagatcagccgggtggaggccgaggacgtgggcgtgtactactgcgtgcaggccacccagttccccatcaccttcggccagggcacccggctggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggctgggcgactactacaactactacggcatggacgtgtggggccagggcaccaccgtgaccgtgagcagc

SEQ ID NO: 430 - DNA, 759 bp, Synthetic construct
  source 1..759: mol_type = other DNA
gacatcgtgatgacccagacccccctgagcagccccgtgaccctgggccagcccgccagcatcagctgccggagcagccagagcctggtgcacagcgacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcaggccatccagttccccatcaccttcggccagggcacccggctggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcaccaccgcctacctgcagtggagcagcctgaaggccagcgacaccgccatgtactactgcgcccggcaccccagctacggcagcggcagccccaacttcgactactggggccagggcaccctggtgaccgtgagcagc

SEQ ID NO: 431 - DNA, 759 bp, Synthetic construct
  source 1..759: mol_type = other DNA
gacatcgtgatgacccagacccccctgagcagccccgtgaccctgggccagcccgccagcatcagctgccggagcagccagagcctggtgtacagcgacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagcacccggcaggtgttcgagcccgccctggagaccgacttcgtgggcggcggcgtgggctacacctacaagtgcgtggtggagcagatcgccaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcaccaccgcctacctgcagtggagcagcctgaaggccagcgacaccgccatgtactactgcgcccggcaccccagctacggcagcggcagccccaacttcgactactggggccagggcaccctggtgaccgtgagcagc

SEQ ID NO: 432 - DNA, 750 bp, Synthetic construct
  source 1..750: mol_type = other DNA
gacgtggtgatgacccagagccccctgagcctgcccgtgaccctgggccagcccgccagcatcagctgccggagcagccagagcctggtgtacagcgacggcaacgcctacctgaactggttccagcagcggcccggccagagcccccggcggctgatctacaaggtgagcaactgggacagcggcgtgcccgaccggttcagcggcagcggcagcggcaccgacttcaccctgaagatccggcgggtggaggccgaggacgtgggcgcctactactgcatgcagggcatccactgcaccttcggccccggcaccaaggtgcacatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcacggcgactacagcaacatcgacgccttcgacatctggggccagggcaccatggtgaccgtgagcagc

SEQ ID NO: 433 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggcaacaccttcctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcagctgacccagttccccctgaccttcggcggcggcaccaaggtggagatccggggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggaccggcaccaccgactactacagcggcatggacgtgtggggccagggcaccaccgtgaccgtgagcagc

SEQ ID NO: 434 - DNA, 762 bp, Synthetic construct
  source 1..762: mol_type = other DNA
gacgtggtgatgacccagagccccctgagcctgcccgtgaccctgggccagcccgccagcatcagctgccggagcagccagagcctggtgtacagcgacggcaacacctacctgaactggttccagcagcggcccggccagagcccccggcggctgatctacaaggtgagcaactgggacagcggcgtgcccgaccggttcagcggcagcggcagcggcaccgacttcaccctgaagatcagccgggtggaggccgaggacgtgggcgtgtactactgcatgcagggcacccactggcccagcatcaccttcggccagggcacccggctggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcacggcgacttctggagcggctactacagcttcgactactggggccagggcaccctggtgaccgtgagcagc

SEQ ID NO: 435 - DNA, 759 bp, Synthetic construct
  source 1..759: mol_type = other DNA
gacatcgtgatgacccagaccccccacagcagccccgtgaccctgggccagcccgccagcatcagctgccggagcagccagagcctggtgagccgggacggcaacacctacctgagctggctgcagcagcggcccggccagcccccccggctgctgatctacaagatcagcaaccggttcagcggcgtgcccaaccggttcagcggcagcggcgccggcaccgacttcaccctgaagatcagccgggtgaaggccgaggacgtgggcgtgtactactgcatgcaggccacccagttccccctgaccttcggccagggcacccggctggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtgtggggccagggcaccaccgtgaccgtgagcagc

SEQ ID NO: 436 - DNA, 759 bp, Synthetic construct
  source 1..759: mol_type = other DNA
gacatcgtgatgacccagacccccctgagcagccccgtgaccctgggccagcccgccagcatcagctgccggagcagccagagcctggtgagccgggacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggtggaggacgtgggcgtgtactactgcatgcaggccacccagttccccatcaccttcggccagggcacccggctggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacaccaagtacagccccagcttccagggccaggtgaccatcagcgccgacaagagcatcagcaccgcctacctgcagtggagcagcctgaaggccagcgacaccgccatgtactactgcgcccggcagggcgacttctggagcggcaacggcggcatggacgtgtggggccagggcaccaccgtgaccgtgagcagc

SEQ ID NO: 437 - DNA, 762 bp, Synthetic construct
  source 1..762: mol_type = other DNA
gacatcgtgatgacccagacccccctgagcagccccgtgaccctgggccagcccgccagcatcagctgccggagcagccagagcctggtgcacagcgacggcaacaccttcctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcagggcacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccaactactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcacggcgactactacgccagcgagagcagcgccttcgacatctggggccagggcaccatggtgaccgtgagcagc

SEQ ID NO: 438 - DNA, 756 bp, Synthetic construct
  source 1..756: mol_type = other DNA
gacatcgtgatgacccagagccccctgagcagccccgtgaccctgggccagcccgccagcatcagctgccggtacagccagagcctggtgcaccgggacggcaacacctacctgagctggctgcagcagcggcccggccagcccccccggctggtgatccacaagatcagcaaccggttcagcggcgtgcccgaccggttcagcggcagcggcgccggcaccgacttcaccctgaagatcagccgggtggaggccgaggacgtgggcttctactactgcatgcagaccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcgactgggtgcggcagatgcccggcaagggcctggagtggatgggcagcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgccagcggctactactacggcatggacgtgtggggccagggcaccaccgtgaccgtgagcagc

SEQ ID NO: 439 - DNA, 744 bp, Synthetic construct
  source 1..744: mol_type = other DNA
gacatcgtgatgacccagacccccctgagcagccccgtgaccctgggccagcccgccagcatcagctgccggagcagccagagcctggtgcacagcgacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcaggccacccagttccccctgaccttcggcggcggcaccaaggtggagatcaagggcggcggcagcagccggagcagcagcagcggcggcggcggcagcggcggcggcggccaggtgcagctggtgcagagcggcgccgaggtgaagaagcccggcgccagcgtgaaggtgagctgcaccgccagcggctacagcttcagcagctacggcatcagctgggtgcggcaggcccccggccagggcctggagtggatgggctggatcagcgcctacaacggccacacccggtacgcccagaagctgcagggccgggtgaccatgaccagcgacaccagcaccagcaccgcctacatggagctgcggagcctgcggagcgacgacaccgccgtgtactactgcgcccgggccagcagctggtacttcgactgctggggccagggcaccctggtgaccgtgagcagc

SEQ ID NO: 440 - DNA, 744 bp, Synthetic construct
  source 1..744: mol_type = other DNA
gagatcgtgatgacccagacccccctgagcagccccgtgaccctgggccagcccgccagcatcagctgcaagagcagccagagcctggtgcacagcgacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcagaccacctacttccccctgaccttcggcggcggcaccaaggtggagatcaagggcggcggcagcagccggagcagcagcagcggcggcggcggcagcggcggcggcggccaggtgcagctggtgcagagcggcgccgaggtgaagaagcccggcgccagcgtgaaggtgagctgcaaggccagcggctacaccttcaccagctacggcatcagctgggtgcggcaggcccccggccagggcctggagtggatgggctggatcaacgcctacaacggcaacaccaactacgcccagaagctgcggggccgggtgaccatgaccaccgacaccttcaccagcaccgccgacatggagctgcggagcctgcggagcgacgacaccgccgtgtactactgcgcccgggccagcaccgccatgggcgactactggggccagggcaccctggtgaccgtgagcagc

SEQ ID NO: 441 - DNA, 762 bp, Synthetic construct
  source 1..762: mol_type = other DNA
gacatcgtgatgacccagacccccctgagcagccccgtgaccctgggccagcccgccagcatcagctgccggagcagccagagcctggtgtacagcgacggcaacacctacctgagctggctgcaccagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcctgtactactgcatgcagaccacccagttccccctgaccttcggcggcggcaccaaggtgaagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccaactactggatcgcctgggtgcggcagatgcccggcaagggcctggagtggatgggcatcatcttccccggcgacagcgacacccggtacagccccgccttccagggccaggtgaccatcagcgccgacaagagcatcagcaccgcctacctgcactggagcagcctgaaggccagcgacaccgccatgtactactgcgcccggcaccgggactacaccagcggcggccccgacgccttcgacgtgtggggccagggcaccatggtgaccgtgagcagc

SEQ ID NO: 442 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggcaacacctacctgagctggctgcagcagcggcccggccagcccccccggctgctgatctacaagctgagcaaccggttcagcggcatccccgacagccggcggttcgtgagcggcgaggccagcgagggcgacgccgtgggcggcaccgtggactacttctacacctgcctgaagatgcagatcgccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcgcctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcaacctgaaggccagcgacaccgccatgtactactgcgcccggaccggcagctactacaactactgcggcatggacgtgtggggccagggcaccaccgtgaccgtgagcagc

SEQ ID NO: 443 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgtaccgggacggcaacacctacctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccaactactggatcgcctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcacccggcagggcgactactacgacagcagcggccccgactactggggccagggcaccctggtgaccgtgagcagc

SEQ ID NO: 444 - DNA, 735 bp, Synthetic construct
  source 1..735: mol_type = other DNA
aagatcgtgatgacccagacccccctgagcagccccgtgaccctgggccagcccgccagcatcagctgccggagcagccagagcctggtgcacagcgacggcaacacctacctgagctggttccagcagcggcccggccagcccccccggctgctgatcaacaagatcagcaaccggttcagcggcgtgcccgaccggttcagcggcagcggcgccggcaccgacttcaccctgaagatcagccgggtggaggccgaggacgtgggcgtgtactactgcatgcaggccacccagttccccctgaccttcggcggcggcaccaaggtggagatcggcggcggcagcagccggagcagcagcagcggcggcggcggcagcggcggcggcggccaggtgcagctggtggagagcgccggcggcgtggtgcagcccggccggagcctgcggctgagctgcgccgccagcggcttcaccttcagcagctacggcatgcactgggtgcggcaggcccccggcaagggcctggagtgggtggccgtgatctggcacgacggcagcaagaagtactacgaggacagcgtgaagggccggttcaccatcagccgggacaacagcaagaacaccctgtacctgcagatgaacagcctgcgggccgaggacaccgccgtgtactactgcgcccggaactggttcttcgactactggggccagggcaccctggtgaccgtgagcagc

SEQ ID NO: 445 - DNA, 747 bp, Synthetic construct
  source 1..747: mol_type = other DNA
agcagcgagctgacccaggaccccgccgtgagcgtggccctgggccagaccgtgcggatcacctgccagggcgacagcctgcggagctactacgccagctggtaccagcagaagcccggccaggcccccgtgctggtgatctacggcaagaacaaccggcccagcggcatccccgcccggttcagcggcagcgacagcggcaacaccgccagcctgaccatcaccggcgcccaggccgaggacgaggccgactactactgcaacagccgggacagcagcggcaaccacgtggtgttcggcggcggcaccaagctgaccgtgctgggcggcggcagcagccggagcagcagcagcggcggcggcggcagcggcggcggcggccaggtgcagctggtggagagcggcggcggcctggtgaagcccggcggcagcctgcggctgagctgcgccgccagcggcttcaccttcagcgactactacatgagctggatccggcaggcccccggcaagggcctggagtgggtgagctacatcagcagcagcggcaccaccatctactacgccgacagcgtgaagggccggttcaccatcagccgggacaacgccaagaacagcctgtacctgcagatgaacagcctgcgggccgaggacaccgccgtgtactactgcgcccgggacctgtactacggcggcaacagctactacttcgactactggggccagggcaccctggtgaccgtgagcagc

SEQ ID NO: 446 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggcaacacctacctgagctggctgcagcagcggcccggccagcccccccggctgctgctgtacaagaacagcaaccggttcagcggcgtgcccgaccggttcagcggcagcggcgccggcaccgacttcaccctgaagatcagccgggtggaggccgaggacgtgggcgtgtactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggggcatcatcggcatctggtacgtgccccggggccaggacatgagccccgacggcaccaagcggggctacctgagcgagccctggagcatgttccagggccaggtgaccatcagcgccgacaagagcatccggaccgcctacctgcagtggagcagcctgaaggccagcgacaccgccatgtactactgcgcccggatcggcgaccactaccactacaacggcatggacgtgtggggccagggcaccaccgtgaccgtgagcagc

SEQ ID NO: 447 - DNA, 756 bp, Synthetic construct
  source 1..756: mol_type = other DNA
gacatcgtgatgacccagaccccccccagcagccccgtgacccggggccagcccgccagcatcagctgccggagcagccagagcctggtgcacagcgacggcaagacctacctgagctggctgcagcagcggcccggccagcccccccggctgctgatctacaagatcagcaaccggttcagcggcgtgcccggccggttcagcggcagcggcgccggcaccgacttcaccctgaagatcagccgggtggaggccgaggacgtgggcgtgtactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcagcagctactggatcaactgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggcggagcctgaaggccagcgacaccgccatctactactgcgcccgggtgggcgactactacagctactacggcatggacgtgtggggccagggcaccaccgtgaccgtgagcagc

SEQ ID NO: 448 - DNA, 756 bp, Synthetic construct
  source 1..756: mol_type = other DNA
gacatcgtgatgacccagacccccctgagcagccccgtgaccctgggccagcccgccagcatcagctgccggagccggcagagcctgctgcacagcgacggcaacacctacctgagctggctgcagcagcggcccggccagcccccccggctgctgatctacaagctgagcaaccgggtgagcggcgtgcccgaccggttcagcggcagcggcgccggcaccgacttcaccctgaagatcagccgggtggaggccgaggacgtgggcgtgtactactgcatgcagagcacccagttccccctgaccttcggcggcggcaccaaggtgaagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccaactactggatcaactgggtgcggcagatgcccggcaagggcctggagtggatgggcatcatctaccccggcgacagcgacacccggtacagccccagcttccagggccaggtgaccatcagcgccgacaagagcatcaacaccgcctacctgcagtggagcagcctgaaggccagcgacaccgccatgtactactgcgcccggcagggcggccactactactacagcggcatggacgtgtggggccagggcaccaccgtgaccgtgagcagc

SEQ ID NO: 449 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgaccggaacacctacctgagctggctgcagcagcggcccggccagcccccccggctgctgatctacaaggtgagcaaccggttcagcggcgtgcccgagcggttcagcggcagcggcaccggcaccgacttcaccctgaagatcagccgggtggaggccgaggacgtgggcgtgtactactgcgtgcaggagaccctgttccccatcaccatcggccagggcacccggctggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagccggaacagcttcaccaactactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggaccggcagctacagctactactacggcatggacgtgtggggccagggcaccaccgtgaccgtgagcagc

SEQ ID NO: 450 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggcaacaccttcctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcatctactactgcatgcaggccacccagttccccctgaccttcggcggcggcaccaaggtggagatcaagggcggcggcagcagccggagcagcagcagcggcggcggcggcagcggcggcggcggcggcgtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgatcatcagcgccgacaagagcatcagcaccgcctacctgcagtggagcagcctgaaggccagcgacaccgccatgtactactgcgcccggaccggcgactactacagctaccacggcatggacgtgtggggccagggcaccaccgtgaccgtgagcagc

SEQ ID NO: 451 - DNA, 765 bp, Synthetic construct
  source 1..765: mol_type = other DNA
gacatcgtgctgacccagacccccctgagcagccccgtgaccctgggccagcccgccagcatcagctgccggagcagccagagcctggtgcacagcgacggccacacctacctgagctggctgcagcagcggcccggccagcccccccggctgctgatctacaagatcagcaaccggttcagcggcgtgcccgaccggttcagcggcagcggcgccggcaccgacttcaccctgaagatcagccgggtgggcgccgaggacgtgggcgtgtactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagagcggcgagagcctgaagatcagctgcaagggcagcggctacagcttcaccaacttctggatcggctgggtgcggcagatgcccggcaagggcctggagtggatgggcatcatctaccccggcgacagcgacacccggtacagccccagcttccagggccaggtgaccatcagcgccgacaagagcatcagcaccacctacctgcagtggagcagcctgaaggccagcgacaccgccatgtactactgcgcccggcaccccccctacagcggcagctactacgccgacgccttcgacatctggggccagggcaccatggtgaccgtgagcagc

SEQ ID NO: 452 - DNA, 765 bp, Synthetic construct
  source 1..765: mol_type = other DNA
gacatcgtgatgacccagacccccctgagcagccccgtgaccctgggccagcccgccagcatcagctgccggagcagccagagcctggtgagcagcgacggcaacacctacctgagctggctgcagcagcggcccggccagcccccccggctgctgatctacaagatcagcaacctgttcagcggcgtgcccgaccggttcagcggcagcggcgccggcaccgacttcaccctgaagatcagccgggtggaggccgaggacgtgggcctgtactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagcttctggatcggctgggtgcggcagatgcccggcaagggcctggagtggatgggcatcatctaccccggcgacagcgacacccggtacagccccagcttccggggcctggtgaccatcagcgccgacaagagcatcagcaccgcctacctgcactggagcagcctgaaggccagcgacaccgccatcttctactgcgcccggcaccccccctacagcggcagctactacgccgacgccttcgacatctggggccagggcaccatggtgaccgtgagcagc

SEQ ID NO: 453 - DNA, 756 bp, Synthetic construct
  source 1..756: mol_type = other DNA
gaccagatccccgtggccatgagcaccatccagagcagctgcccccggctgagcagcagcagccagcccagcgtgctgaccgtgctgcacggcagcgacggcaacaccttcctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatccaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggaccggcgactaccacaactactacggcatggacgtgtggggccagggcaccaccgtgaccgtgagcagc

SEQ ID NO: 454 - DNA, 756 bp, Synthetic construct
  source 1..756: mol_type = other DNA
gacgtggtgatgacccagagccccctgagcctgcccgtgaccctgggccagcccgccagcatcagctgccggagccggcagagcctggtgtacagcgacggcagcacctacctgaactggttccagcagcggcccggccagagcccccggcggctgatctacaaggtgagcaactgggacagcggcgtgcccgaccggttcagcgccagcggcagcggcaccgacttcaccctgaagatcagccgggtggaggccgaggacgacggcgtgtaccactgcatgcagggcacccactggcccctgctggccttcggcggcggcaccaaggtggagatcggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccagctactggatcggctgggtgcggcagatgcccggcaagggcctggagtggatgggcatcatctaccccgccgacagcgacacccggtacagccccagcttccagggccaggtgaccatcagcgccgacaagagcctgaacaccgcctacctgcagtggcggagcctgaaggccagcgacaccgccatgtactactgcgcccggatcggcgacttctactactacagcggcatggacgtgtggggccagggcaccaccgtgaccgtgagcagc

SEQ ID NO: 455 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggcaacaccttcctgagctggctgcagcagcggcccggccagcccccccggctgctgatctacaagatcagcaaccggttcagcggcgtgcccgaccggttcagcggcagcggcgccggcaccgacttcaccctgaagatcagccgggtggaggccgaggacgtgggcgtgtactactgcatgcaggccacccagttccccctgaccttcggcggcggcaccaaggtggagatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacatcttcaccagctactggatcgcctgggtgcggcagatgcccggcaagggcctggagtggatgggcatcatctaccccggcgacagcgacacccggtacagccccagcttccagggccagatcaccatcagcgccgacaagagcatcagcaccgcctacctgcagtggagcagcctgaaggccagcgacaccgccatgtactactgcgcccgggtgggcaccaccaactactactacggcatggacgtgtggggccagggcaccagcgtgaccgtgagcagc

SEQ ID NO: 456 - DNA, 756 bp, Synthetic construct
  source 1..756: mol_type = other DNA
gacatcgtgatgacccagacccccctgagcagccccgtgaccctgggccagcccgccagcatcagctgccggagcagccagagcctggtgcacagcgacggccacacctacctgagctggctgcagcagcggcccggccagcccccccggctgctgttctacaagatcagcaaccggttcagcggcgtgcccgaccggttcagcggcagcggcgccggcaccgacttcaccctgaagatcagccgggtggaggccgaggacgtgggcatctactactgcatgcaggccacccagttccccctgaccttcggcggcggcaccaaggtggacatcaagggcggcggcagcagccggagcagcagcagcggcggcggcggcagcggcggcggcggcgaggtgcagctggtgcagagcggcgccgaggtgaagaagcccggcgagagcctgaagatcagctgcaagggcagcggctacagcttcaccacctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggatcggcgactactacagctacagcggcctggacgtgtggggccagggcaccaccgtgaccgtgagcagc

SEQ ID NO: 457 - DNA, 765 bp, Synthetic construct
  source 1..765: mol_type = other DNA
cagcccgtgctgacccagcccaccagcctgagcgccagccccggcgccagcgcccggctgacctgcaccctgcggagcggcatcaacctgggcagctaccggatcttctggtaccagcagaagcccgagagccccccccggtacctgctgagctactacagcgacagccggaagcaccagggcagcggcgtgcccagccggttcagcggcagcaaggacgccagcagcaacgccggcatcctggtgatcagcggcctgcagagcgaggacgaggccgactactactgcatcttctggcacagcagcgcctgggtgttcggcggcggcaccaagctgaccgtgggcggcggcagcagccggagcagcagcagcggcggcggcggcagcggcggcggcggccaggtgcagctgcagcagagcggccccggcctgatgaagcccagccagaccctgagcctgacctgcgccatcagcggcgacagcgtgagcagcaacagcgtgagctggaactggatccggcagagccccagccggggcctggagtggctgggccggacctactaccggttcaagtggttcaacgactacgccgtgagcgtgaagagccggatcaccatcaaccccgacaccagcaagaaccagttcagcctgcggctgaacagcgtgacccccgaggacaccgccctgtactactgcgcccggatcgacatctggaacgacgtgttcgactactggggccagggcaccctggtgaccgtgagcagc

SEQ ID NO: 458 - protein, 287 aa, Synthetic construct
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGSSRSSSSGGGGSGGGGMGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 459 - protein, 248 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGSSRSSSSGGGGSGGGGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 460 - protein, 248 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGSSRSSSSGGGGSGGGGEVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 461 - protein, 251 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIKGGGSSRSSSSGGGGSGGGGEVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 462 - protein, 686 aa, Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSSRSSSSGGGGSGGGGQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 463 - protein, 246 aa, Synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGSSRSSSSGGGGSGGGGQVQLQQPGAELVKAGASVKMSCKASGYTFTSYNMHWVKQTPGRGLEWIGAIYPGNGDTSYNQKFKGKATLTADKSSSTAYMQLSSLTSEDSAVYYCARSTYYGGDTYFNVWGAGTTVTVSA

SEQ ID NO: 464 - protein, 246 aa, Synthetic construct
QIVLSQSPAIITASPGEKVTMTCRASTSASYIHWFQQKPTSSPKPWIYATSNLASGVPSRFSGSGSGTTYSMTISSLEAEDAATYYCQQWTSNPPTFGGGTKLEIKGGGSSRSSSSGGGGSGGGGQVQLQQPGAELKKPGASVKVSCKASGYTFTSYNMHWVKQTPGRGLEWTGAIYPGNGDTSYNQKFKGKTTLTADKSSSTAYMELSSLRSEDTAVYYCARSTYYGGDWYFNVWGAGTTVTVSA

SEQ ID NO: 465 - protein, 246 aa, Synthetic construct
QIVLSQSPAIITASPGEKVTMTCRASTSVSYIHWFQQKPTSSPKPWIYATSNLASGVPSRFSGSGSGTTYSMTISSLEAEDAATYYCQQWTSNPPTFGGGTKLEIKGGGSSRSSSSGGGGSGGGGQVQLQQPGAELKKPGASVKVSCKASGYTFTSYNMHWVKQTPGRGLEWIGAIYPGNGDTSYNQKFKGKTTLTADKSSSTAYMELSSLRSEDTAVYYCARSTYYGGDWYFNVWGAGTTVTVSA

SEQ ID NO: 466 - protein, 247 aa, Synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGSSRSSSSGGGGSGGGGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 467 - protein, 247 aa, Synthetic construct
DIVLTQSPAIITASPGEKVTMTCRASSSVNYMDWYQKKPGSSPKPWIYATSNLASGVPSRFSGSGSGTTYSMTISSLEAEDAATYYCQQWSFNPPTFGGGTKLEIKGGGSSRSSSSGGGGSGGGGEVQLQQSGAELKKPGASVKVSCKASGYTFTSYNMHWVKQTPGQGLEWIGAIYPGNGDTSYNQKFKGKTTLTADKSSSTAYMELSSLRSEDTAVYYCARSNYYGSSYWFFDVWGTGTTVTVSS

SEQ ID NO: 468 - protein, 247 aa, Synthetic construct
QIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELGGGSSRSSSSGGGGSGGGGQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTGTGSS

SEQ ID NO: 469 - protein, 253 aa, Synthetic construct
DIVMTQTPLSSPVTLGQPASISCRSSQSLVYSDGNTYLSWLQQRPGQPPRLLIYKISNRFSGVPDRFSGSGAGTDFTLKISTRQVFEPALETDFVGGGVGYTYKCVVEQIAKGGGSSRSSSSGGGGSGGGGEVQLVQSGAEVKKPGESLKISCKGSGYSFTSYWIGWVRQMPGKGLEWMGIIYPGDSDTRYRPSFQGQVTISADKSITTAYLQWSSLKASDTAMYYCARHPSYGSGSPNFDYWGQGTLVTVSS

SEQ ID NO: 470 - protein, 230 aa, Synthetic construct
EIVMTQSPATLSVSPGERATLSCRASQSVSSNLAWYQQKPGQAPRLLISGASTRATGIPARFSGSGSGTEFTLTISSLQSEDFAVYYCHQYNDWSLTFGGGTKVEIKGGGSSRSSSSGGGGSGGGGSCKGSGYSFTSYWIGWVRQMPGKGLEWMGIIYPGDSDTRYSPSFQGQVTISADKSISTAYLQWSSLKASDTAMYYCARMGDFWSGYYTRGMDVWGQGTTVTVSS

SEQ ID NO: 471 - protein, 252 aa, Synthetic construct
DIVMTQTPLSSPVTLGQPASISCRSSQRLVHSDGHTYLSWLQQRPGQPPRLLISKVSNRFSGVPDRFSGSGAGTDFTLKISRVEAEDVGIYYCMQATQFPLTFGGGTKVEIKGGGSSRSSSSGGGGSGGGGEVQLVQSGAEVKKPGESLKISCKGSGYSFTNYWIVWVRQMPGKGLEWMGIIYPGDSDTRYSPSFQGQVTISADKSISTAYLQWSSLKASDTAMYYCARHGDYYYYYGMDVWGQGTTVTVSS

SEQ ID NO: 472 - protein, 253 aa, Synthetic construct
DIVMTQTPLSSPVTLGQPASISCRSSQSLVYSDGNTYLSWLQQRPGQPPRLLFYKISNRFSGVPDRFSGSGAGTDFTLKISRVEAEDVGVYYCVQATQFPLTFGGGTKVEIKGGGSSRSSSSGGGGSGGGGEVQLVQSGAEVKKPGESLKISCKGSGYSFTSYWIGWVRQMSGKGLEWMGIIYPGDSDTRYSPSFQGQVTISADKSISTAYLQWSSLKASDTAIYYCARHPSYGSGSPNFDYWGQGTLVTVSS

SEQ ID NO: 473 - protein, 252 aa, Synthetic construct
DVVMTQSPLSLSVTLGQPASISCRSSQSLVYNDGDTYLNWFQQRPGQSPRLLIYKVSNWDSGVPDRFSGSGSGTDFTLKVSRVEAEDVGVYYCMQGTHWPLTFGGGTKVEIKGGGSSRSSSSGGGGSGGGGEVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARQGDYYHYSGMDVWGQGTTVTVSS

SEQ ID NO: 474 - protein, 252 aa, Synthetic construct
DIVMTQTPLSSPVTLGQPASISCRSSQSLVHSDGKTYLSWLQQRPGQPPRLLIYKSSNRFSGVPDRFSGSGAGTDFTLKISRVEAEDVGVYYCVQATQFPITFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGDYYNYYGMDVWGQGTTVTVSS

SEQ ID NO: 475 - protein, 253 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQAIQFPITFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 476 - protein, 253 aa, Synthetic construct
DIVMTQTPLSSPVTLGQPASISCRSSQSLVYSDGNTYLSWLQQRPGQPPRLLIYKISNRFSGVPDRFSGSGAGTDFTLKISTRQVFEPALETDFVGGGVGYTYKCVVEQIAKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 477 - protein, 250 aa, Synthetic construct
DVVMTQSPLSLPVTLGQPASISCRSSQSLVYSDGNAYLNWFQQRPGQSPRRLIYKVSNWDSGVPDRFSGSGSGTDFTLKIRRVEAEDVGAYYCMQGIHCTFGPGTKVHIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDYSNIDAFDIWGQGTMVTVSS

SEQ ID NO: 478 - protein, 252 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQLTQFPLTFGGGTKVEIRGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGTTDYYSGMDVWGQGTTVTVSS

SEQ ID NO: 479 - protein, 254 aa, Synthetic construct
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPSITFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDFWSGYYSFDYWGQGTLVTVSS

SEQ ID NO: 480 - protein, 253 aa, Synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 481 - protein, 253 aa, Synthetic construct
DIVMTQTPLSSPVTLGQPASISCRSSQSLVSRDGNTYLSWLQQRPGQPPRLLIYKISNRFSGVPDRFSGSGAGTDFTLKISRVEVEDVGVYYCMQATQFPITFGQGTRLEIKGGGSSRSSSSGGGGSGGGGEVQLVQSGAEVKKPGESLKISCKGSGYSFTSYWIGWVRQMPGKGLEWMGIIYPGDSDTKYSPSFQGQVTISADKSISTAYLQWSSLKASDTAMYYCARQGDFWSGNGGMDVWGQGTTVTVSS

SEQ ID NO: 482 - protein, 254 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQGTQFPLTFGGGTKVEIKGGGSSRSSSSGGGGSGGGGEVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGDYYASESSAFDIWGQGTMVTVSS

SEQ ID NO: 483 - protein, 252 aa, Synthetic construct
DIVMTQSPLSSPVTLGQPASISCRYSQSLVHRDGNTYLSWLQQRPGQPPRLVIHKISNRFSGVPDRFSGSGAGTDFTLKISRVEAEDVGFYYCMQTTQFPLTFGGGTKVEIKGGGSSRSSSSGGGGSGGGGEVQLVQSGAEVKKPGESLKISCKGSGYSFTSYWIDWVRQMPGKGLEWMGSIYPGDSDTRYSPSFQGQVTISADKSISTAYLQWSSLKASDTAMYYCARQGASGYYYGMDVWGQGTTVTVSS

SEQ ID NO: 484 - protein, 248 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIKGGGSSRSSSSGGGGSGGGGQVQLVQSGAEVKKPGASVKVSCTASGYSFSSYGISWVRQAPGQGLEWMGWISAYNGHTRYAQKLQGRVTMTSDTSTSTAYMELRSLRSDDTAVYYCARASSWYFDCWGQGTLVTVSS

SEQ ID NO: 485 - protein, 248 aa, Synthetic construct
EIVMTQTPLSSPVTLGQPASISCKSSQSLVHSDGNTYLSWLQQRPGQPPRLLIYKISNRFSGVPDRFSGSGAGTDFTLKISRVEAEDVGVYYCMQTTYFPLTFGGGTKVEIKGGGSSRSSSSGGGGSGGGGQVQLVQSGAEVKKPGASVKVSCKASGYTFTSYGISWVRQAPGQGLEWMGWINAYNGNTNYAQKLRGRVTMTTDTFTSTADMELRSLRSDDTAVYYCARASTAMGDYWGQGTLVTVSS

SEQ ID NO: 486 - protein, 254 aa, Synthetic construct
DIVMTQTPLSSPVTLGQPASISCRSSQSLVYSDGNTYLSWLHQRPGQPPRLLIYKISNRFSGVPDRFSGSGAGTDFTLKISRVEAEDVGLYYCMQTTQFPLTFGGGTKVKIKGGGSSRSSSSGGGGSGGGGEVQLVQSGAEVKKPGESLKISCKGSGYSFTNYWIAWVRQMPGKGLEWMGIIFPGDSDTRYSPAFQGQVTISADKSISTAYLHWSSLKASDTAMYYCARHRDYTSGGPDAFDVWGQGTMVTVSS

SEQ ID NO: 487 - protein, 252 aa, Synthetic construct
DIVMTQTPLSSPVTLGQPASISCRSSQSLVHSDGNTYLSWLQQRPGQPPRLLIYKLSNRFSGIPDSRRFVSGEASEGDAVGGTVDYFYTCLKMQIATQFPLTFGGGTKVEIKGGGSSRSSSSGGGGSGGGGEVQLVQSGAEVKKPGESLKISCKGSGYSFTSYWIAWVRQMPGKGLEWMGIIYPGDSDTRYSPSFQGQVTISADKSISTAYLQWSNLKASDTAMYYCARTGSYYNYCGMDVWGQGTTVTVSS

SEQ ID NO: 488 - protein, 252 aa, Synthetic construct
DIVMTQTPLSSPVTLGQPASISCRSSQSLVYRDGNTYLSWLQQRPGQPPRLLIYKISNRFSGVPDRFSGSGAGTDFTLKISRVEAEDVGVYYCMQATQFPLTFGGGTKVEIKGGGSSRSSSSGGGGSGGGGEVQLVQSGAEVKKPGESLKISCKGSGYSFTNYWIAWVRQMPGKGLEWMGIIYPGDSDTRYSPSFQGQVTISADKSISTAYLQWSSLKASDTAMYYCTRQGDYYDSSGPDYWGQGTLVTVSS

SEQ ID NO: 489 - protein, 245 aa, Synthetic construct
KIVMTQTPLSSPVTLGQPASISCRSSQSLVHSDGNTYLSWFQQRPGQPPRLLINKISNRFSGVPDRFSGSGAGTDFTLKISRVEAEDVGVYYCMQATQFPLTFGGGTKVEIGGGSSRSSSSGGGGSGGGGQVQLVESAGGVVQPGRSLRLSCAASGFTFSSYGMHWVRQAPGKGLEWVAVIWHDGSKKYYEDSVKGRFTISRDNSKNTLYLQMNSLRAEDTAVYYCARNWFFDYWGQGTLVTVSS

SEQ ID NO: 490 - protein, 249 aa, Synthetic construct
SSELTQDPAVSVALGQTVRITCQGDSLRSYYASWYQQKPGQAPVLVIYGKNNRPSGIPARFSGSDSGNTASLTITGAQAEDEADYYCNSRDSSGNHVVFGGGTKLTVLGGGSSRSSSSGGGGSGGGGQVQLVESGGGLVKPGGSLRLSCAASGFTFSDYYMSWIRQAPGKGLEWVSYISSSGTTIYYADSVKGRFTISRDNAKNSLYLQMNSLRAEDTAVYYCARDLYYGGNSYYFDYWGQGTLVTVSS

SEQ ID NO: 491 - protein, 252 aa, Synthetic construct
DIVMTQTPLSSPVTLGQPASISCRSSQSLVHSDGNTYLSWLQQRPGQPPRLLLYKNSNRFSGVPDRFSGSGAGTDFTLKISRVEAEDVGVYYCMQATQFPLTFGGGTKVEIKGGGSSRSSSSGGGGSGGGGEVQLVQSGAEVKKPGESLKISCKGSGYSFTSYWGIIGIWYVPRGQDMSPDGTKRGYLSEPWSMFQGQVTISADKSIRTAYLQWSSLKASDTAMYYCARIGDHYHYNGMDVWGQGTTVTVSS

SEQ ID NO: 492 - protein, 252 aa, Synthetic construct
DIVMTQTPPSSPVTRGQPASISCRSSQSLVHSDGKTYLSWLQQRPGQPPRLLIYKISNRFSGVPGRFSGSGAGTDFTLKISRVEAEDVGVYYCMQATQFPLTFGGGTKVEIKGGGSSRSSSSGGGGSGGGGEVQLVQSGAEVKKPGESLKISCKGSGYSFSSYWINWVRQMPGKGLEWMGIIYPGDSDTRYSPSFQGQVTISADKSISTAYLQWRSLKASDTAIYYCARVGDYYSYYGMDVWGQGTTVTVSS

SEQ ID NO: 493 - protein, 252 aa, Synthetic construct
DIVMTQTPLSSPVTLGQPASISCRSRQSLLHSDGNTYLSWLQQRPGQPPRLLIYKLSNRVSGVPDRFSGSGAGTDFTLKISRVEAEDVGVYYCMQSTQFPLTFGGGTKVKIKGGGSSRSSSSGGGGSGGGGEVQLVQSGAEVKKPGESLKISCKGSGYSFTNYWINWVRQMPGKGLEWMGIIYPGDSDTRYSPSFQGQVTISADKSINTAYLQWSSLKASDTAMYYCARQGGHYYYSGMDVWGQGTTVTVSS

SEQ ID NO: 494 - protein, 252 aa, Synthetic construct
DIVMTQTPLSSPVTLGQPASISCRSSQSLVHSDRNTYLSWLQQRPGQPPRLLIYKVSNRFSGVPERFSGSGTGTDFTLKISRVEAEDVGVYYCVQETLFPITIGQGTRLEIKGGGSSRSSSSGGGGSGGGGEVQLVQSGAEVKKPGESLKISCKGSRNSFTNYWIGWVRQMPGKGLEWMGIIYPGDSDTRYSPSFQGQVTISADKSISTAYLQWSSLKASDTAMYYCARTGSYSYYYGMDVWGQGTTVTVSS

SEQ ID NO: 495 - protein, 252 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGIYYCMQATQFPLTFGGGTKVEIKGGGSSRSSSSGGGGSGGGGGVQLVQSGAEVKKPGESLKISCKGSGYSFTSYWIGWVRQMPGKGLEWMGIIYPGDSDTRYSPSFQGQVIISADKSISTAYLQWSSLKASDTAMYYCARTGDYYSYHGMDVWGQGTTVTVSS

SEQ ID NO: 496 - protein, 255 aa, Synthetic construct
DIVLTQTPLSSPVTLGQPASISCRSSQSLVHSDGHTYLSWLQQRPGQPPRLLIYKISNRFSGVPDRFSGSGAGTDFTLKISRVGAEDVGVYYCMQATQFPLTFGGGTKVEIKGGGSSRSSSSGGGGSGGGGEVQLVQSGAEVKKSGESLKISCKGSGYSFTNFWIGWVRQMPGKGLEWMGIIYPGDSDTRYSPSFQGQVTISADKSISTTYLQWSSLKASDTAMYYCARHPPYSGSYYADAFDIWGQGTMVTVSS

SEQ ID NO: 497 - protein, 255 aa, Synthetic construct
DIVMTQTPLSSPVTLGQPASISCRSSQSLVSSDGNTYLSWLQQRPGQPPRLLIYKISNLFSGVPDRFSGSGAGTDFTLKISRVEAEDVGLYYCMQATQFPLTFGGGTKVEIKGGGSSRSSSSGGGGSGGGGEVQLVQSGAEVKKPGESLKISCKGSGYSFTSFWIGWVRQMPGKGLEWMGIIYPGDSDTRYSPSFRGLVTISADKSISTAYLHWSSLKASDTAIFYCARHPPYSGSYYADAFDIWGQGTMVTVSS

SEQ ID NO: 498 - protein, 252 aa, Synthetic construct
DQIPVAMSTIQSSCPRLSSSSQPSVLTVLHGSDGNTFLSWLQQRPGQPPRLLIYKISNRFSGVPDRFSGSGAGTDFTLKISRVEAEDVGVYYCIQATQFPLTFGGGTKVEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGDYHNYYGMDVWGQGTTVTVSS

SEQ ID NO: 499 - protein, 252 aa, Synthetic construct
DVVMTQSPLSLPVTLGQPASISCRSRQSLVYSDGSTYLNWFQQRPGQSPRRLIYKVSNWDSGVPDRFSASGSGTDFTLKISRVEAEDDGVYHCMQGTHWPLLAFGGGTKVEIGGGSSRSSSSGGGGSGGGGEVQLVQSGAEVKKPGESLKISCKGSGYSFTSYWIGWVRQMPGKGLEWMGIIYPADSDTRYSPSFQGQVTISADKSLNTAYLQWRSLKASDTAMYYCARIGDFYYYSGMDVWGQGTTVTVSS

SEQ ID NO: 500 - protein, 252 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQATQFPLTFGGGTKVEIKGGGSSRSSSSGGGGSGGGGEVQLVQSGAEVKKPGESLKISCKGSGYIFTSYWIAWVRQMPGKGLEWMGIIYPGDSDTRYSPSFQGQITISADKSISTAYLQWSSLKASDTAMYYCARVGTTNYYYGMDVWGQGTSVTVSS

SEQ ID NO: 501 - protein, 252 aa, Synthetic construct
DIVMTQTPLSSPVTLGQPASISCRSSQSLVHSDGHTYLSWLQQRPGQPPRLLFYKISNRFSGVPDRFSGSGAGTDFTLKISRVEAEDVGIYYCMQATQFPLTFGGGTKVDIKGGGSSRSSSSGGGGSGGGGEVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARIGDYYSYSGLDVWGQGTTVTVSS

SEQ ID NO: 502 - protein, 255 aa, Synthetic construct
QPVLTQPTSLSASPGASARLTCTLRSGINLGSYRIFWYQQKPESPPRYLLSYYSDSRKHQGSGVPSRFSGSKDASSNAGILVISGLQSEDEADYYCIFWHSSAWVFGGGTKLTVGGGSSRSSSSGGGGSGGGGQVQLQQSGPGLMKPSQTLSLTCAISGDSVSSNSVSWNWIRQSPSRGLEWLGRTYYRFKWFNDYAVSVKSRITINPDTSKNQFSLRLNSVTPEDTALYYCARIDIWNDVFDYWGQGTLVTVSS

SEQ ID NO: 503 - protein, 10 aa, Synthetic construct
RASSSVSYIH

SEQ ID NO: 504 - protein, 7 aa, Synthetic construct
ATSNLAS

SEQ ID NO: 505 - protein, 9 aa, Synthetic construct
QQWTSNPPT

SEQ ID NO: 506 - protein, 5 aa, Synthetic construct
SYNMH

SEQ ID NO: 507 - protein, 17 aa, Synthetic construct
AIYPGNGDTSYNQKFKG

SEQ ID NO: 508 - protein, 12 aa, Synthetic construct
STYYGGDWYFNV

SEQ ID NO: 509 - protein, 11 aa, Synthetic construct
RASQSVSSYLA

SEQ ID NO: 510 - protein, 7 aa, Synthetic construct
DASNRAT

SEQ ID NO: 511 - protein, 9 aa, Synthetic construct
QQRSNWPIT

SEQ ID NO: 512 - protein, 6 aa, Synthetic construct
NDYAMH

SEQ ID NO: 513 - protein, 17 aa, Synthetic construct
TISWNSGSIGYADSVKG

SEQ ID NO: 514 - protein, 13 aa, Synthetic construct
DIQYGNYYYGMDV

SEQ ID NO: 515 - protein, 9 aa, Synthetic construct
QQRSDWPLT

SEQ ID NO: 516 - protein, 17 aa, Synthetic construct
TISWNSGTIGYADSVKG

SEQ ID NO: 517 - protein, 5 aa, Synthetic construct
YHAMH

SEQ ID NO: 518 - protein, 16 aa, Synthetic construct
IIGTGGVTYYADSVKG

SEQ ID NO: 519 - protein, 17 aa, Synthetic construct
DYYGAGSFYDGLYGMDV

SEQ ID NO: 520 - protein, 16 aa, Synthetic construct
RSSKSLLHSNGITYLY

SEQ ID NO: 521 - protein, 7 aa, Synthetic construct
QMSNLVS

SEQ ID NO: 522 - protein, 9 aa, Synthetic construct
AQNLELPYT

SEQ ID NO: 523 - protein, 6 aa, Synthetic construct
GYAFSY

SEQ ID NO: 524 - protein, 8 aa, Synthetic construct
FPGDGDTD

SEQ ID NO: 525 - protein, 10 aa, Synthetic construct
NVFDGYWLVY

SEQ ID NO: 526 - protein, 128 aa, Synthetic construct
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 527 - protein, 107 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 528 - protein, 107 aa, Synthetic construct; identical to SEQ ID NO: 527

SEQ ID NO: 529 - protein, 107 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIK

SEQ ID NO: 530 - protein, 219 aa, Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 531 - protein, 106 aa, Synthetic construct [sequence omitted]

SEQ ID NO: 532 - protein, 106 aa, Synthetic construct
QIVLSQSPAIITASPGEKVTMTCRASTSASYIHWFQQKPTSSPKPWIYATSNLASGVPSRFSGSGSGTTYSMTISSLEAEDAATYYCQQWTSNPPTFGGGTKLEIK

SEQ ID NO: 533 - protein, 106 aa, Synthetic construct
QIVLSQSPAIITASPGEKVTMTCRASTSVSYIHWFQQKPTSSPKPWIYATSNLASGVPSRFSGSGSGTTYSMTISSLEAEDAATYYCQQWTSNPPTFGGGTKLEIK

SEQ ID NO: 534 - protein, 106 aa, Synthetic construct
QIVLSQSPAIITASPGEKVTMTCRASTSVSYIHWFQQKPGSSPKPWIYATSNLASGVPSRFSGSGSGTTYSMTISSLEAEDAATYYCQQWTSNPPTFGGGTKLEIK

SEQ ID NO: 535 - protein, 106 aa, Synthetic construct
QIVLSQSPAIITASPGEKVTMTCRASSSVSYIHWFQQKPGSSPKPWIYATSNLASGVPSRFSGSGSGTTYSMTISSLEAEDAATYYCQQWTSNPPTFGGGTKLEIK

SEQ ID NO: 536 - protein, 106 aa, Synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 537 - protein, 106 aa, Synthetic construct
DIVLTQSPAIITASPGEKVTMTCRASSSVNYMDWYQKKPGSSPKPWIYATSNLASGVPSRFSGSGSGTTYSMTISSLEAEDAATYYCQQWSFNPPTFGGGTKLEIK

SEQ ID NO: 538 - protein, 105 aa, Synthetic construct
QIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLEL

SEQ ID NO: 539 - protein, 112 aa, Synthetic construct
DIVMTQTPLSSPVTLGQPASISCRSSQSLVYSDGNTYLSWLQQRPGQPPRLLIYKISNRFSGVPDRFSGSGAGTDFTLKISTRQVFEPALETDFVGGGVGYTYKCVVEQIAK

SEQ ID NO: 540 - protein, 107 aa, Synthetic construct
EIVMTQSPATLSVSPGERATLSCRASQSVSSNLAWYQQKPGQAPRLLISGASTRATGIPARFSGSGSGTEFTLTISSLQSEDFAVYYCHQYNDWSLTFGGGTKVEIK

SEQ ID NO: 541 - protein, 112 aa, Synthetic construct
DIVMTQTPLSSPVTLGQPASISCRSSQRLVHSDGHTYLSWLQQRPGQPPRLLISKVSNRFSGVPDRFSGSGAGTDFTLKISRVEAEDVGIYYCMQATQFPLTFGGGTKVEIK

SEQ ID NO: 542 - protein, 112 aa, Synthetic construct
DIVMTQTPLSSPVTLGQPASISCRSSQSLVYSDGNTYLSWLQQRPGQPPRLLFYKISNRFSGVPDRFSGSGAGTDFTLKISRVEAEDVGVYYCVQATQFPLTFGGGTKVEIK

SEQ ID NO: 543 - protein, 112 aa, Synthetic construct
DVVMTQSPLSLSVTLGQPASISCRSSQSLVYNDGDTYLNWFQQRPGQSPRLLIYKVSNWDSGVPDRFSGSGSGTDFTLKVSRVEAEDVGVYYCMQGTHWPLTFGGGTKVEIK

SEQ ID NO: 544 - protein, 112 aa, Synthetic construct
DIVMTQTPLSSPVTLGQPASISCRSSQSLVHSDGKTYLSWLQQRPGQPPRLLIYKSSNRFSGVPDRFSGSGAGTDFTLKISRVEAEDVGVYYCVQATQFPITFGQGTRLEIK

SEQ ID NO: 545 - protein, 112 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQAIQFPITFGQGTRLEIK

SEQ ID NO: 546 - protein, 112 aa, Synthetic construct; identical to SEQ ID NO: 539

SEQ ID NO: 547 - protein, 110 aa, Synthetic construct
DVVMTQSPLSLPVTLGQPASISCRSSQSLVYSDGNAYLNWFQQRPGQSPRRLIYKVSNWDSGVPDRFSGSGSGTDFTLKIRRVEAEDVGAYYCMQGIHCTFGPGTKVHIK

SEQ ID NO: 548 - protein, 112 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQLTQFPLTFGGGTKVEIR

SEQ ID NO: 549 - protein, 113 aa, Synthetic construct
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPSITFGQGTRLEIK

SEQ ID NO: 550 - protein, 112 aa, Synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 551 - protein, 112 aa, Synthetic construct
DIVMTQTPLSSPVTLGQPASISCRSSQSLVSRDGNTYLSWLQQRPGQPPRLLIYKISNRFSGVPDRFSGSGAGTDFTLKISRVEVEDVGVYYCMQATQFPITFGQGTRLEIK

SEQ ID NO: 552 - protein, 112 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQGTQFPLTFGGGTKVEIK

SEQ ID NO: 553 - protein, 112 aa, Synthetic construct
DIVMTQSPLSSPVTLGQPASISCRYSQSLVHRDGNTYLSWLQQRPGQPPRLVIHKISNRFSGVPDRFSGSGAGTDFTLKISRVEAEDVGFYYCMQTTQFPLTFGGGTKVEIK

SEQ ID NO: 554 - protein, 112 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIK

SEQ ID NO: 555 - protein, 112 aa, Synthetic construct
EIVMTQTPLSSPVTLGQPASISCKSSQSLVHSDGNTYLSWLQQRPGQPPRLLIYKISNRFSGVPDRFSGSGAGTDFTLKISRVEAEDVGVYYCMQTTYFPLTFGGGTKVEIK

SEQ ID NO: 556 - protein, 112 aa, Synthetic construct
DIVMTQTPLSSPVTLGQPASISCRSSQSLVYSDGNTYLSWLHQRPGQPPRLLIYKISNRFSGVPDRFSGSGAGTDFTLKISRVEAEDVGLYYCMQTTQFPLTFGGGTKVKIK

SEQ ID NO: 557 - protein, 112 aa, Synthetic construct
DIVMTQTPLSSPVTLGQPASISCRSSQSLVHSDGNTYLSWLQQRPGQPPRLLIYKLSNRFSGIPDSRRFVSGEASEGDAVGGTVDYFYTCLKMQIATQFPLTFGGGTKVEIK

SEQ ID NO: 558 - protein, 112 aa, Synthetic construct
DIVMTQTPLSSPVTLGQPASISCRSSQSLVYRDGNTYLSWLQQRPGQPPRLLIYKISNRFSGVPDRFSGSGAGTDFTLKISRVEAEDVGVYYCMQATQFPLTFGGGTKVEIK

SEQ ID NO: 559 - protein, 111 aa, Synthetic construct
KIVMTQTPLSSPVTLGQPASISCRSSQSLVHSDGNTYLSWFQQRPGQPPRLLINKISNRFSGVPDRFSGSGAGTDFTLKISRVEAEDVGVYYCMQATQFPLTFGGGTKVEI

SEQ ID NO: 560 - protein, 108 aa, Synthetic construct
SSELTQDPAVSVALGQTVRITCQGDSLRSYYASWYQQKPGQAPVLVIYGKNNRPSGIPARFSGSDSGNTASLTITGAQAEDEADYYCNSRDSSGNHVVFGGGTKLTVL

SEQ ID NO: 561 - protein, 112 aa, Synthetic construct
DIVMTQTPLSSPVTLGQPASISCRSSQSLVHSDGNTYLSWLQQRPGQPPRLLLYKNSNRFSGVPDRFSGSGAGTDFTLKISRVEAEDVGVYYCMQATQFPLTFGGGTKVEIK

SEQ ID NO: 562 - protein, 112 aa, Synthetic construct
DIVMTQTPPSSPVTRGQPASISCRSSQSLVHSDGKTYLSWLQQRPGQPPRLLIYKISNRFSGVPGRFSGSGAGTDFTLKISRVEAEDVGVYYCMQATQFPLTFGGGTKVEIK

SEQ ID NO: 563 - protein, 112 aa, Synthetic construct
DIVMTQTPLSSPVTLGQPASISCRSRQSLLHSDGNTYLSWLQQRPGQPPRLLIYKLSNRVSGVPDRFSGSGAGTDFTLKISRVEAEDVGVYYCMQSTQFPLTFGGGTKVKIK

SEQ ID NO: 564 - protein, 121 aa, Synthetic construct
EVQLVQSGAEVKKPGESLKISCKGSGYSFTNYWINWVRQMPGKGLEWMGIIYPGDSDTRYSPSFQGQVTISADKSINTAYLQWSSLKASDTAMYYCARQGGHYYYSGMDVWGQGTTVTVSS

SEQ ID NO: 565 - protein, 112 aa, Synthetic construct
DIVMTQTPLSSPVTLGQPASISCRSSQSLVHSDRNTYLSWLQQRPGQPPRLLIYKVSNRFSGVPERFSGSGTGTDFTLKISRVEAEDVGVYYCVQETLFPITIGQGTRLEIK

SEQ ID NO: 566 - protein, 121 aa, Synthetic construct
EVQLVQSGAEVKKPGESLKISCKGSRNSFTNYWIGWVRQMPGKGLEWMGIIYPGDSDTRYSPSFQGQVTISADKSISTAYLQWSSLKASDTAMYYCARTGSYSYYYGMDVWGQGTTVTVSS

SEQ ID NO: 567 - protein, 112 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGIYYCMQATQFPLTFGGGTKVEIK

SEQ ID NO: 568 - protein, 112 aa, Synthetic construct
DIVLTQTPLSSPVTLGQPASISCRSSQSLVHSDGHTYLSWLQQRPGQPPRLLIYKISNRFSGVPDRFSGSGAGTDFTLKISRVGAEDVGVYYCMQATQFPLTFGGGTKVEIK

SEQ ID NO: 569 - protein, 112 aa, Synthetic construct
DIVMTQTPLSSPVTLGQPASISCRSSQSLVSSDGNTYLSWLQQRPGQPPRLLIYKISNLFSGVPDRFSGSGAGTDFTLKISRVEAEDVGLYYCMQATQFPLTFGGGTKVEIK

SEQ ID NO: 570 - protein, 112 aa, Synthetic construct
DQIPVAMSTIQSSCPRLSSSSQPSVLTVLHGSDGNTFLSWLQQRPGQPPRLLIYKISNRFSGVPDRFSGSGAGTDFTLKISRVEAEDVGVYYCIQATQFPLTFGGGTKVEIK

SEQ ID NO: 571 - protein, 112 aa, Synthetic construct
DVVMTQSPLSLPVTLGQPASISCRSRQSLVYSDGSTYLNWFQQRPGQSPRRLIYKVSNWDSGVPDRFSASGSGTDFTLKISRVEAEDDGVYHCMQGTHWPLLAFGGGTKVEI

SEQ ID NO: 572 - protein, 112 aa, Synthetic construct
DIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQATQFPLTFGGGTKVEIK

SEQ ID NO: 573 - protein, 112 aa, Synthetic construct
DIVMTQTPLSSPVTLGQPASISCRSSQSLVHSDGHTYLSWLQQRPGQPPRLLFYKISNRFSGVPDRFSGSGAGTDFTLKISRVEAEDVGIYYCMQATQFPLTFGGGTKVDIK

SEQ ID NO: 574 - protein, 114 aa, Synthetic construct
QPVLTQPTSLSASPGASARLTCTLRSGINLGSYRIFWYQQKPESPPRYLLSYYSDSRKHQGSGVPSRFSGSKDASSNAGILVISGLQSEDEADYYCIFWHSSAWVFGGGTKLTV

SEQ ID NO: 575 - skipped

SEQ ID NO: 576 - protein, 140 aa, Synthetic construct
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 577 - protein, 122 aa, Synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 578 - protein, 122 aa, Synthetic construct
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 579 - protein, 125 aa, Synthetic construct
EVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 580 - protein, 125 aa, Synthetic construct; identical to SEQ ID NO: 579

SEQ ID NO: 581 - protein, 121 aa, Synthetic construct
QVQLQQPGAELVKAGASVKMSCKASGYTFTSYNMHWVKQTPGRGLEWIGAIYPGNGDTSYNQKFKGKATLTADKSSSTAYMQLSSLTSEDSAVYYCARSTYYGGDTYFNVWGAGTTVTVSA

SEQ ID NO: 582 - protein, 121 aa, Synthetic construct
QVQLQQPGAELKKPGASVKVSCKASGYTFTSYNMHWVKQTPGRGLEWTGAIYPGNGDTSYNQKFKGKTTLTADKSSSTAYMELSSLRSEDTAVYYCARSTYYGGDWYFNVWGAGTTVTVSA

SEQ ID NO: 583 - protein, 121 aa, Synthetic construct
QVQLQQPGAELKKPGASVKVSCKASGYTFTSYNMHWVKQTPGRGLEWIGAIYPGNGDTSYNQKFKGKTTLTADKSSSTAYMELSSLRSEDTAVYYCARSTYYGGDWYFNVWGAGTTVTVSA

SEQ ID NO: 584 - protein, 122 aa, Synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 585 - protein, 122 aa, Synthetic construct
EVQLQQSGAELKKPGASVKVSCKASGYTFTSYNMHWVKQTPGQGLEWIGAIYPGNGDTSYNQKFKGKTTLTADKSSSTAYMELSSLRSEDTAVYYCARSNYYGSSYWFFDVWGTGTTVTVSS

SEQ ID NO: 586 - protein, 123 aa, Synthetic construct
QVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTGTGSS

SEQ ID NO: 587 - protein, 122 aa, Synthetic construct
EVQLVQSGAEVKKPGESLKISCKGSGYSFTSYWIGWVRQMPGKGLEWMGIIYPGDSDTRYRPSFQGQVTISADKSITTAYLQWSSLKASDTAMYYCARHPSYGSGSPNFDYWGQGTLVTVSS

SEQ ID NO: 588 - protein, 104 aa, Synthetic construct
SCKGSGYSFTSYWIGWVRQMPGKGLEWMGIIYPGDSDTRYSPSFQGQVTISADKSISTAYLQWSSLKASDTAMYYCARMGDFWSGYYTRGMDVWGQGTTVTVSS

SEQ ID NO: 589 - protein, 121 aa, Synthetic construct
EVQLVQSGAEVKKPGESLKISCKGSGYSFTNYWIVWVRQMPGKGLEWMGIIYPGDSDTRYSPSFQGQVTISADKSISTAYLQWSSLKASDTAMYYCARHGDYYYYYGMDVWGQGTTVTVSS

SEQ ID NO: 590 - protein, 122 aa, Synthetic construct
EVQLVQSGAEVKKPGESLKISCKGSGYSFTSYWIGWVRQMSGKGLEWMGIIYPGDSDTRYSPSFQGQVTISADKSISTAYLQWSSLKASDTAIYYCARHPSYGSGSPNFDYWGQGTLVTVSS

SEQ ID NO: 591 - protein, 121 aa, Synthetic construct
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARQGDYYHYSGMDVWGQGTTVTVSS

SEQ ID NO: 592 - protein, 121 aa, Synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGDYYNYYGMDVWGQGTTVTVSS

SEQ ID NO: 593 - protein, 122 aa, Synthetic construct
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 594 - protein, 122 aa, Synthetic construct; identical to SEQ ID NO: 593

SEQ ID NO: 595 - protein, 121 aa, Synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDYSNIDAFDIWGQGTMVTVSS

SEQ ID NO: 596 - protein, 121 aa, Synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGTTDYYSGMDVWGQGTTVTVSS

SEQ ID NO: 597 - protein, 122 aa, Synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDFWSGYYSFDYWGQGTLVTVSS

SEQ ID NO: 598 - protein, 122 aa, Synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 599 - protein, 122 aa, Synthetic construct
EVQLVQSGAEVKKPGESLKISCKGSGYSFTSYWIGWVRQMPGKGLEWMGIIYPGDSDTKYSPSFQGQVTISADKSISTAYLQWSSLKASDTAMYYCARQGDFWSGNGGMDVWGQGTTVTVSS

SEQ ID NO: 600 - protein, 123 aa, Synthetic construct
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGDYYASESSAFDIWGQGTMVTVSS

SEQ ID NO: 601 - protein, 121 aa, Synthetic construct
EVQLVQSGAEVKKPGESLKISCKGSGYSFTSYWIDWVRQMPGKGLEWMGSIYPGDSDTRYSPSFQGQVTISADKSISTAYLQWSSLKASDTAMYYCARQGASGYYYGMDVWGQGTTVTVSS

SEQ ID NO: 602 - protein, 117 aa, Synthetic construct
QVQLVQSGAEVKKPGASVKVSCTASGYSFSSYGISWVRQAPGQGLEWMGWISAYNGHTRYAQKLQGRVTMTSDTSTSTAYMELRSLRSDDTAVYYCARASSWYFDCWGQGTLVTVSS

SEQ ID NO: 603 - protein, 117 aa, Synthetic construct
QVQLVQSGAEVKKPGASVKVSCKASGYTFTSYGISWVRQAPGQGLEWMGWINAYNGNTNYAQKLRGRVTMTTDTFTSTADMELRSLRSDDTAVYYCARASTAMGDYWGQGTLVTVSS

SEQ ID NO: 604 - protein, 123 aa, Synthetic construct
EVQLVQSGAEVKKPGESLKISCKGSGYSFTNYWIAWVRQMPGKGLEWMGIIFPGDSDTRYSPAFQGQVTISADKSISTAYLHWSSLKASDTAMYYCARHRDYTSGGPDAFDVWGQGTMVTVSS

SEQ ID NO: 605 - protein, 121 aa, Synthetic construct
EVQLVQSGAEVKKPGESLKISCKGSGYSFTSYWIAWVRQMPGKGLEWMGIIYPGDSDTRYSPSFQGQVTISADKSISTAYLQWSNLKASDTAMYYCARTGSYYNYCGMDVWGQGTTVTVSS

SEQ ID NO: 606 - protein, 121 aa, Synthetic construct
EVQLVQSGAEVKKPGESLKISCKGSGYSFTNYWIAWVRQMPGKGLEWMGIIYPGDSDTRYSPSFQGQVTISADKSISTAYLQWSSLKASDTAMYYCTRQGDYYDSSGPDYWGQGTLVTVSS

SEQ ID NO: 607 - protein, 115 aa, Synthetic construct
QVQLVESAGGVVQPGRSLRLSCAASGFTFSSYGMHWVRQAPGKGLEWVAVIWHDGSKKYYEDSVKGRFTISRDNSKNTLYLQMNSLRAEDTAVYYCARNWFFDYWGQGTLVTVSS

SEQ ID NO: 608 - protein, 122 aa, Synthetic construct
QVQLVESGGGLVKPGGSLRLSCAASGFTFSDYYMSWIRQAPGKGLEWVSYISSSGTTIYYADSVKGRFTISRDNAKNSLYLQMNSLRAEDTAVYYCARDLYYGGNSYYFDYWGQGTLVTVSS

SEQ ID NO: 609 - protein, 121 aa, Synthetic construct
EVQLVQSGAEVKKPGESLKISCKGSGYSFTSYWGIIGIWYVPRGQDMSPDGTKRGYLSEPWSMFQGQVTISADKSIRTAYLQWSSLKASDTAMYYCARIGDHYHYNGMDVWGQGTTVTVSS

SEQ ID NO: 610 - protein, 121 aa, Synthetic construct
EVQLVQSGAEVKKPGESLKISCKGSGYSFSSYWINWVRQMPGKGLEWMGIIYPGDSDTRYSPSFQGQVTISADKSISTAYLQWRSLKASDTAIYYCARVGDYYSYYGMDVWGQGTTVTVSS

SEQ ID NO: 611 - protein, 121 aa, Synthetic construct
GVQLVQSGAEVKKPGESLKISCKGSGYSFTSYWIGWVRQMPGKGLEWMGIIYPGDSDTRYSPSFQGQVIISADKSISTAYLQWSSLKASDTAMYYCARTGDYYSYHGMDVWGQGTTVTVSS

SEQ ID NO: 612 - protein, 124 aa, Synthetic construct
EVQLVQSGAEVKKSGESLKISCKGSGYSFTNFWIGWVRQMPGKGLEWMGIIYPGDSDTRYSPSFQGQVTISADKSISTTYLQWSSLKASDTAMYYCARHPPYSGSYYADAFDIWGQGTMVTVSS

SEQ ID NO: 613 - protein, 124 aa, Synthetic construct
EVQLVQSGAEVKKPGESLKISCKGSGYSFTSFWIGWVRQMPGKGLEWMGIIYPGDSDTRYSPSFRGLVTISADKSISTAYLHWSSLKASDTAIFYCARHPPYSGSYYADAFDIWGQGTMVTVSS

SEQ ID NO: 614 - protein, 121 aa, Synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGDYHNYYGMDVWGQGTTVTVSS

SEQ ID NO: 615 - protein, 121 aa, Synthetic construct
EVQLVQSGAEVKKPGESLKISCKGSGYSFTSYWIGWVRQMPGKGLEWMGIIYPADSDTRYSPSFQGQVTISADKSLNTAYLQWRSLKASDTAMYYCARIGDFYYYSGMDVWGQGTTVTVSS

SEQ ID NO: 616 - protein, 121 aa, Synthetic construct
EVQLVQSGAEVKKPGESLKISCKGSGYIFTSYWIAWVRQMPGKGLEWMGIIYPGDSDTRYSPSFQGQITISADKSISTAYLQWSSLKASDTAMYYCARVGTTNYYYGMDVWGQGTSVTVSS

SEQ ID NO: 617 - protein, 121 aa, Synthetic construct
EVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARIGDYYSYSGLDVWGQGTTVTVSS

SEQ ID NO: 618 - protein, 122 aa, Synthetic construct
QVQLQQSGPGLMKPSQTLSLTCAISGDSVSSNSVSWNWIRQSPSRGLEWLGRTYYRFKWFNDYAVSVKSRITINPDTSKNQFSLRLNSVTPEDTALYYCARIDIWNDVFDYWGQGTLVTVSS

SEQ ID NO: 619 - protein, 409 aa, Synthetic construct
MALPVTALLLPLALLLHAARPGSMDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGSSRSSSSGGGGSGGGGMGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 620 - protein, 370 aa, Synthetic construct
MALPVTALLLPLALLLHAARPGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGSSRSSSSGGGGSGGGGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 621 - protein, 370 aa, Synthetic construct
MALPVTALLLPLALLLHAARPGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGSSRSSSSGGGGSGGGGEVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 622 - protein, 373 aa, Synthetic construct
MALPVTALLLPLALLLHAARPGSEIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIKGGGSSRSSSSGGGGSGGGGEVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 623 - protein, 808 aa, Synthetic construct
MALPVTALLLPLALLLHAARPGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSSRSSSSGGGGSGGGGQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 624 - protein, 368 aa, Synthetic construct
MALPVTALLLPLALLLHAARPGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGSSRSSSSGGGGSGGGGQVQLQQPGAELVKAGASVKMSCKASGYTFTSYNMHWVKQTPGRGLEWIGAIYPGNGDTSYNQKFKGKATLTADKSSSTAYMQLSSLTSEDSAVYYCARSTYYGGDTYFNVWGAGTTVTVSAASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 625 - protein, 368 aa, Synthetic construct
MALPVTALLLPLALLLHAARPGSQIVLSQSPAIITASPGEKVTMTCRASTSASYIHWFQQKPTSSPKPWIYATSNLASGVPSRFSGSGSGTTYSMTISSLEAEDAATYYCQQWTSNPPTFGGGTKLEIKGGGSSRSSSSGGGGSGGGGQVQLQQPGAELKKPGASVKVSCKASGYTFTSYNMHWVKQTPGRGLEWTGAIYPGNGDTSYNQKFKGKTTLTADKSSSTAYMELSSLRSEDTAVYYCARSTYYGGDWYFNVWGAGTTVTVSAASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 626 - protein, 368 aa, Synthetic construct
MALPVTALLLPLALLLHAARPGSQIVLSQSPAIITASPGEKVTMTCRASTSVSYIHWFQQKPTSSPKPWIYATSNLASGVPSRFSGSGSGTTYSMTISSLEAEDAATYYCQQWTSNPPTFGGGTKLEIKGGGSSRSSSSGGGGSGGGGQVQLQQPGAELKKPGASVKVSCKASGYTFTSYNMHWVKQTPGRGLEWIGAIYPGNGDTSYNQKFKGKTTLTADKSSSTAYMELSSLRSEDTAVYYCARSTYYGGDWYFNVWGAGTTVTVSAASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 627 - protein, 369 aa, Synthetic construct
MALPVTALLLPLALLLHAARPGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGSSRSSSSGGGGSGGGGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 628 - protein, 369 aa, Synthetic construct
MALPVTALLLPLALLLHAARPGSDIVLTQSPAIITASPGEKVTMTCRASSSVNYMDWYQKKPGSSPKPWIYATSNLASGVPSRFSGSGSGTTYSMTISSLEAEDAATYYCQQWSFNPPTFGGGTKLEIKGGGSSRSSSSGGGGSGGGGEVQLQQSGAELKKPGASVKVSCKASGYTFTSYNMHWVKQTPGQGLEWIGAIYPGNGDTSYNQKFKGKTTLTADKSSSTAYMELSSLRSEDTAVYYCARSNYYGSSYWFFDVWGTGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 629 - protein, 369 aa, Synthetic construct
MALPVTALLLPLALLLHAARPGSQIVLSQSPTILSASPGEKVTMTCRASSSVSYMDWYQQKPGSSPKPWIYATSNLASGVPARFSGSGSGTSYSLTISRVEAEDAATYYCQQWISNPPTFGAGTKLELGGGSSRSSSSGGGGSGGGGQVQLQQPGAELVKPGASVKMSCKTSGYTFTSYNVHWVKQTPGQGLEWIGAIYPGNGDTSFNQKFKGKATLTADKSSSTVYMQLSSLTSEDSAVYYCARSNYYGSSYVWFFDVWGAGTTGTGSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 630 - protein, 375 aa, Synthetic construct
MALPVTALLLPLALLLHAARPGSDIVMTQTPLSSPVTLGQPASISCRSSQSLVYSDGNTYLSWLQQRPGQPPRLLIYKISNRFSGVPDRFSGSGAGTDFTLKISTRQVFEPALETDFVGGGVGYTYKCVVEQIAKGGGSSRSSSSGGGGSGGGGEVQLVQSGAEVKKPGESLKISCKGSGYSFTSYWIGWVRQMPGKGLEWMGIIYPGDSDTRYRPSFQGQVTISADKSITTAYLQWSSLKASDTAMYYCARHPSYGSGSPNFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 631 - protein, 352 aa, Synthetic construct
MALPVTALLLPLALLLHAARPGSEIVMTQSPATLSVSPGERATLSCRASQSVSSNLAWYQQKPGQAPRLLISGASTRATGIPARFSGSGSGTEFTLTISSLQSEDFAVYYCHQYNDWSLTFGGGTKVEIKGGGSSRSSSSGGGGSGGGGSCKGSGYSFTSYWIGWVRQMPGKGLEWMGIIYPGDSDTRYSPSFQGQVTISADKSISTAYLQWSSLKASDTAMYYCARMGDFWSGYYTRGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 632 - protein, 374 aa, Synthetic construct
MALPVTALLLPLALLLHAARPGSDIVMTQTPLSSPVTLGQPASISCRSSQRLVHSDGHTYLSWLQQRPGQPPRLLISKVSNRFSGVPDRFSGSGAGTDFTLKISRVEAEDVGIYYCMQATQFPLTFGGGTKVEIKGGGSSRSSSSGGGGSGGGGEVQLVQSGAEVKKPGESLKISCKGSGYSFTNYWIVWVRQMPGKGLEWMGIIYPGDSDTRYSPSFQGQVTISADKSISTAYLQWSSLKASDTAMYYCARHGDYYYYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 633 - protein, 375 aa, Synthetic construct
MALPVTALLLPLALLLHAARPGSDIVMTQTPLSSPVTLGQPASISCRSSQSLVYSDGNTYLSWLQQRPGQPPRLLFYKISNRFSGVPDRFSGSGAGTDFTLKISRVEAEDVGVYYCVQATQFPLTFGGGTKVEIKGGGSSRSSSSGGGGSGGGGEVQLVQSGAEVKKPGESLKISCKGSGYSFTSYWIGWVRQMSGKGLEWMGIIYPGDSDTRYSPSFQGQVTISADKSISTAYLQWSSLKASDTAIYYCARHPSYGSGSPNFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 634 - protein, 374 aa, Synthetic construct
MALPVTALLLPLALLLHAARPGSDVVMTQSPLSLSVTLGQPASISCRSSQSLVYNDGDTYLNWFQQRPGQSPRLLIYKVSNWDSGVPDRFSGSGSGTDFTLKVSRVEAEDVGVYYCMQGTHWPLTFGGGTKVEIKGGGSSRSSSSGGGGSGGGGEVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARQGDYYHYSGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 635 - protein, 374 aa, Synthetic construct
MALPVTALLLPLALLLHAARPGSDIVMTQTPLSSPVTLGQPASISCRSSQSLVHSDGKTYLSWLQQRPGQPPRLLIYKSSNRFSGVPDRFSGSGAGTDFTLKISRVEAEDVGVYYCVQATQFPITFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGDYYNYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 636 - protein, 375 aa, Synthetic construct
MALPVTALLLPLALLLHAARPGSDIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQAIQFPITFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 637 - protein, 375 aa, Synthetic construct
MALPVTALLLPLALLLHAARPGSDIVMTQTPLSSPVTLGQPASISCRSSQSLVYSDGNTYLSWLQQRPGQPPRLLIYKISNRFSGVPDRFSGSGAGTDFTLKISTRQVFEPALETDFVGGGVGYTYKCVVEQIAKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 638 - protein, 372 aa, Synthetic construct
MALPVTALLLPLALLLHAARPGSDVVMTQSPLSLPVTLGQPASISCRSSQSLVYSDGNAYLNWFQQRPGQSPRRLIYKVSNWDSGVPDRFSGSGSGTDFTLKIRRVEAEDVGAYYCMQGIHCTFGPGTKVHIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDYSNIDAFDIWGQGTMVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 639 - protein, 374 aa, Synthetic construct
MALPVTALLLPLALLLHAARPGSDIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQLTQFPLTFGGGTKVEIRGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGTTDYYSGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 640 - protein, 376 aa, Synthetic construct
MALPVTALLLPLALLLHAARPGSDVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPSITFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGDFWSGYYSFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 641 - protein, 375 aa, Synthetic construct
MALPVTALLLPLALLLHAARPGSDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 642 - protein, 375 aa, Synthetic construct
MALPVTALLLPLALLLHAARPGSDIVMTQTPLSSPVTLGQPASISCRSSQSLVSRDGNTYLSWLQQRPGQPPRLLIYKISNRFSGVPDRFSGSGAGTDFTLKISRVEVEDVGVYYCMQATQFPITFGQGTRLEIKGGGSSRSSSSGGGGSGGGGEVQLVQSGAEVKKPGESLKISCKGSGYSFTSYWIGWVRQMPGKGLEWMGIIYPGDSDTKYSPSFQGQVTISADKSISTAYLQWSSLKASDTAMYYCARQGDFWSGNGGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 643 - protein, 376 aa, Synthetic construct
MALPVTALLLPLALLLHAARPGSDIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQGTQFPLTFGGGTKVEIKGGGSSRSSSSGGGGSGGGGEVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGDYYASESSAFDIWGQGTMVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 644 - protein, 374 aa, Synthetic construct
MALPVTALLLPLALLLHAARPGSDIVMTQSPLSSPVTLGQPASISCRYSQSLVHRDGNTYLSWLQQRPGQPPRLVIHKISNRFSGVPDRFSGSGAGTDFTLKISRVEAEDVGFYYCMQTTQFPLTFGGGTKVEIKGGGSSRSSSSGGGGSGGGGEVQLVQSGAEVKKPGESLKISCKGSGYSFTSYWIDWVRQMPGKGLEWMGSIYPGDSDTRYSPSFQGQVTISADKSISTAYLQWSSLKASDTAMYYCARQGASGYYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 645 - protein, 370 aa, Synthetic construct
MALPVTALLLPLALLLHAARPGSDIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIKGGGSSRSSSSGGGGSGGGGQVQLVQSGAEVKKPGASVKVSCTASGYSFSSYGISWVRQAPGQGLEWMGWISAYNGHTRYAQKLQGRVTMTSDTSTSTAYMELRSLRSDDTAVYYCARASSWYFDCWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 646 - protein, 370 aa, Synthetic construct
MALPVTALLLPLALLLHAARPGSEIVMTQTPLSSPVTLGQPASISCKSSQSLVHSDGNTYLSWLQQRPGQPPRLLIYKISNRFSGVPDRFSGSGAGTDFTLKISRVEAEDVGVYYCMQTTYFPLTFGGGTKVEIKGGGSSRSSSSGGGGSGGGGQVQLVQSGAEVKKPGASVKVSCKASGYTFTSYGISWVRQAPGQGLEWMGWINAYNGNTNYAQKLRGRVTMTTDTFTSTADMELRSLRSDDTAVYYCARASTAMGDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 647 - protein, 376 aa, Synthetic construct
MALPVTALLLPLALLLHAARPGSDIVMTQTPLSSPVTLGQPASISCRSSQSLVYSDGNTYLSWLHQRPGQPPRLLIYKISNRFSGVPDRFSGSGAGTDFTLKISRVEAEDVGLYYCMQTTQFPLTFGGGTKVKIKGGGSSRSSSSGGGGSGGGGEVQLVQSGAEVKKPGESLKISCKGSGYSFTNYWIAWVRQMPGKGLEWMGIIFPGDSDTRYSPAFQGQVTISADKSISTAYLHWSSLKASDTAMYYCARHRDYTSGGPDAFDVWGQGTMVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 648 - protein, 374 aa, Synthetic construct
MALPVTALLLPLALLLHAARPGSDIVMTQTPLSSPVTLGQPASISCRSSQSLVHSDGNTYLSWLQQRPGQPPRLLIYKLSNRFSGIPDSRRFVSGEASEGDAVGGTVDYFYTCLKMQIATQFPLTFGGGTKVEIKGGGSSRSSSSGGGGSGGGGEVQLVQSGAEVKKPGESLKISCKGSGYSFTSYWIAWVRQMPGKGLEWMGIIYPGDSDTRYSPSFQGQVTISADKSISTAYLQWSNLKASDTAMYYCARTGSYYNYCGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 649 - protein, 374 aa, Synthetic construct
MALPVTALLLPLALLLHAARPGSDIVMTQTPLSSPVTLGQPASISCRSSQSLVYRDGNTYLSWLQQRPGQPPRLLIYKISNRFSGVPDRFSGSGAGTDFTLKISRVEAEDVGVYYCMQATQFPLTFGGGTKVEIKGGGSSRSSSSGGGGSGGGGEVQLVQSGAEVKKPGESLKISCKGSGYSFTNYWIAWVRQMPGKGLEWMGIIYPGDSDTRYSPSFQGQVTISADKSISTAYLQWSSLKASDTAMYYCTRQGDYYDSSGPDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 650 - protein, 367 aa, Synthetic construct
MALPVTALLLPLALLLHAARPGSKIVMTQTPLSSPVTLGQPASISCRSSQSLVHSDGNTYLSWFQQRPGQPPRLLINKISNRFSGVPDRFSGSGAGTDFTLKISRVEAEDVGVYYCMQATQFPLTFGGGTKVEIGGGSSRSSSSGGGGSGGGGQVQLVESAGGVVQPGRSLRLSCAASGFTFSSYGMHWVRQAPGKGLEWVAVIWHDGSKKYYEDSVKGRFTISRDNSKNTLYLQMNSLRAEDTAVYYCARNWFFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 651 - protein, 371 aa, Synthetic construct
MALPVTALLLPLALLLHAARPGSSSELTQDPAVSVALGQTVRITCQGDSLRSYYASWYQQKPGQAPVLVIYGKNNRPSGIPARFSGSDSGNTASLTITGAQAEDEADYYCNSRDSSGNHVVFGGGTKLTVLGGGSSRSSSSGGGGSGGGGQVQLVESGGGLVKPGGSLRLSCAASGFTFSDYYMSWIRQAPGKGLEWVSYISSSGTTIYYADSVKGRFTISRDNAKNSLYLQMNSLRAEDTAVYYCARDLYYGGNSYYFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 652 - protein, 374 aa, Synthetic construct
MALPVTALLLPLALLLHAARPGSDIVMTQTPLSSPVTLGQPASISCRSSQSLVHSDGNTYLSWLQQRPGQPPRLLLYKNSNRFSGVPDRFSGSGAGTDFTLKISRVEAEDVGVYYCMQATQFPLTFGGGTKVEIKGGGSSRSSSSGGGGSGGGGEVQLVQSGAEVKKPGESLKISCKGSGYSFTSYWGIIGIWYVPRGQDMSPDGTKRGYLSEPWSMFQGQVTISADKSIRTAYLQWSSLKASDTAMYYCARIGDHYHYNGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 653 - protein, 374 aa, Synthetic construct
MALPVTALLLPLALLLHAARPGSDIVMTQTPPSSPVTRGQPASISCRSSQSLVHSDGKTYLSWLQQRPGQPPRLLIYKISNRFSGVPGRFSGSGAGTDFTLKISRVEAEDVGVYYCMQATQFPLTFGGGTKVEIKGGGSSRSSSSGGGGSGGGGEVQLVQSGAEVKKPGESLKISCKGSGYSFSSYWINWVRQMPGKGLEWMGIIYPGDSDTRYSPSFQGQVTISADKSISTAYLQWRSLKASDTAIYYCARVGDYYSYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 654 - protein, 374 aa, Synthetic construct
MALPVTALLLPLALLLHAARPGSDIVMTQTPLSSPVTLGQPASISCRSRQSLLHSDGNTYLSWLQQRPGQPPRLLIYKLSNRVSGVPDRFSGSGAGTDFTLKISRVEAEDVGVYYCMQSTQFPLTFGGGTKVKIKGGGSSRSSSSGGGGSGGGGEVQLVQSGAEVKKPGESLKISCKGSGYSFTNYWINWVRQMPGKGLEWMGIIYPGDSDTRYSPSFQGQVTISADKSINTAYLQWSSLKASDTAMYYCARQGGHYYYSGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 655 - protein, 374 aa, Synthetic construct
MALPVTALLLPLALLLHAARPGSDIVMTQTPLSSPVTLGQPASISCRSSQSLVHSDRNTYLSWLQQRPGQPPRLLIYKVSNRFSGVPERFSGSGTGTDFTLKISRVEAEDVGVYYCVQETLFPITIGQGTRLEIKGGGSSRSSSSGGGGSGGGGEVQLVQSGAEVKKPGESLKISCKGSRNSFTNYWIGWVRQMPGKGLEWMGIIYPGDSDTRYSPSFQGQVTISADKSISTAYLQWSSLKASDTAMYYCARTGSYSYYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 656 - protein, 374 aa, Synthetic construct
MALPVTALLLPLALLLHAARPGSDIVMTQTPLSSPVTLGQPASISCRSSQSLVHSDGNTFLSWLQQRPGQPPRLLIYKISNRFSGVPDRFSGSGAGTDFTLKISRVEAEDVGIYYCMQATQFPLTFGGGTKVEIKGGGSSRSSSSGGGGSGGGGGVQLVQSGAEVKKPGESLKISCKGSGYSFTSYWIGWVRQMPGKGLEWMGIIYPGDSDTRYSPSFQGQVIISADKSISTAYLQWSSLKASDTAMYYCARTGDYYSYHGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 657 - protein, 377 aa, Synthetic construct
MALPVTALLLPLALLLHAARPGSDIVLTQTPLSSPVTLGQPASISCRSSQSLVHSDGHTYLSWLQQRPGQPPRLLIYKISNRFSGVPDRFSGSGAGTDFTLKISRVGAEDVGVYYCMQATQFPLTFGGGTKVEIKGGGSSRSSSSGGGGSGGGGEVQLVQSGAEVKKSGESLKISCKGSGYSFTNFWIGWVRQMPGKGLEWMGIIYPGDSDTRYSPSFQGQVTISADKSISTTYLQWSSLKASDTAMYYCARHPPYSGSYYADAFDIWGQGTMVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 658 - protein, 377 aa, Synthetic construct
MALPVTALLLPLALLLHAARPGSDIVMTQTPLSSPVTLGQPASISCRSSQSLVSSDGNTYLSWLQQRPGQPPRLLIYKISNLFSGVPDRFSGSGAGTDFTLKISRVEAEDVGLYYCMQATQFPLTFGGGTKVEIKGGGSSRSSSSGGGGSGGGGEVQLVQSGAEVKKPGESLKISCKGSGYSFTSFWIGWVRQMPGKGLEWMGIIYPGDSDTRYSPSFRGLVTISADKSISTAYLHWSSLKASDTAIFYCARHPPYSGSYYADAFDIWGQGTMVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 659 - protein, 374 aa, Synthetic construct
MALPVTALLLPLALLLHAARPGSDQIPVAMSTIQSSCPRLSSSSQPSVLTVLHGSDGNTFLSWLQQRPGQPPRLLIYKISNRFSGVPDRFSGSGAGTDFTLKISRVEAEDVGVYYCIQATQFPLTFGGGTKVEIKGGGSSRSSSSGGGGSGGGGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARTGDYHNYYGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 660 - protein, 374 aa, Synthetic construct
MALPVTALLLPLALLLHAARPGSDVVMTQSPLSLPVTLGQPASISCRSRQSLVYSDGSTYLNWFQQRPGQSPRRLIYKVSNWDSGVPDRFSASGSGTDFTLKISRVEAEDDGVYHCMQGTHWPLLAFGGGTKVEIGGGSSRSSSSGGGGSGGGGEVQLVQSGAEVKKPGESLKISCKGSGYSFTSYWIGWVRQMPGKGLEWMGIIYPADSDTRYSPSFQGQVTISADKSLNTAYLQWRSLKASDTAMYYCARIGDFYYYSGMDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 661 - protein, 374 aa, Synthetic construct
MALPVTALLLPLALLLHAARPGSDIVMTQTPLSSPVTLGQPASISCRSSQSLVHSDGNTFLSWLQQRPGQPPRLLIYKISNRFSGVPDRFSGSGAGTDFTLKISRVEAEDVGVYYCMQATQFPLTFGGGTKVEIKGGGSSRSSSSGGGGSGGGGEVQLVQSGAEVKKPGESLKISCKGSGYIFTSYWIAWVRQMPGKGLEWMGIIYPGDSDTRYSPSFQGQITISADKSISTAYLQWSSLKASDTAMYYCARVGTTNYYYGMDVWGQGTSVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 662 - protein, 374 aa, Synthetic construct
MALPVTALLLPLALLLHAARPGSDIVMTQTPLSSPVTLGQPASISCRSSQSLVHSDGHTYLSWLQQRPGQPPRLLFYKISNRFSGVPDRFSGSGAGTDFTLKISRVEAEDVGIYYCMQATQFPLTFGGGTKVDIKGGGSSRSSSSGGGGSGGGGEVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARIGDYYSYSGLDVWGQGTTVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 663 - protein, 377 aa, Synthetic construct
MALPVTALLLPLALLLHAARPGSQPVLTQPTSLSASPGASARLTCTLRSGINLGSYRIFWYQQKPESPPRYLLSYYSDSRKHQGSGVPSRFSGSKDASSNAGILVISGLQSEDEADYYCIFWHSSAWVFGGGTKLTVGGGSSRSSSSGGGGSGGGGQVQLQQSGPGLMKPSQTLSLTCAISGDSVSSNSVSWNWIRQSPSRGLEWLGRTYYRFKWFNDYAVSVKSRITINPDTSKNQFSLRLNSVTPEDTALYYCARIDIWNDVFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 664 - protein, 42 aa, Synthetic construct
KRGRKKLLYIFKQPFMRPVQTTQEEDGCSCRFPEEEEGGCEL

SEQ ID NO: 665 - DNA, 339 bp, Synthetic construct
  source 1..339: mol_type = other DNA
atgggcggcctggaaccgtgcagccgctttctgctgctgccgctgctgctggcggtgagcggcctgcgcccggtgcaggtgcaggcgcagagcgattgcagctgcagcaccgtgagcccgggcgtgctggcgggcattgtgatgggcgatctggtgctgaccgtgctgattgcgctggcggtgtattttctgggccgcctggtgccgcgcggccgcggcgcggcggaagcggcgacccgcaaacagcgcattaccgaaaccgaaagcccgtatcaggaactgcagggccagcgcagcgatgtgtatagcgatctgaacacccagcgcccgtattataaa

SEQ ID NO: 666 - protein, 113 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 667 - protein, 304 aa, Synthetic construct [sequence omitted]

SEQ ID NO: 668 - protein, 304 aa, Synthetic construct
MSLLVVSMACVGFFLLQGAWPHEGVHRKPSLLAHPGRLVKSEETVILQCWSDVMFEHFLLHREGMFNDTLRLIGEHHDGVSKANFSISRMKQDLAGTYRCYGSVTHSPYQLSAPSDPLDIVIIGLYEKPSLSAQPGPTVLAGENVTLSCSSRSSYDMYHLSREGEAHERRLPAGTKVNGTFQANFPLGPATHGGTYRCFGSFRDSPYEWSKSSDPLLVSVTGNPSNSWPSPTEPSSETGNPRHLHVLIGTSVVKIPFTILLFFLLHRWCSDKKNAAVMDQEPAGNRTVNSEDSDEQDHQEVSYA

SEQ ID NO: 669 - protein, 295 aa, Synthetic construct
MSLMVISMACVGFFWLQGAWPHEGFRRKPSLLAHPGRLVKSEETVILQCWSDVMFEHFLLHREGTFNDTLRLIGEHIDGVSKANFSIGRMRQDLAGTYRCYGSVPHSPYQFSAPSDPLDIVITGLYEKPSLSAQPGPTVLAGESVTLSCSSWSSYDMYHLSTEGEAHERRFSAGPKVNGTFQADFPLGPATQGGTYRCFGSFHDSPYEWSKSSDPLLVSVTGNPSNSWPSPTEPSSKTGNPRHLHVLIGTSVVKLPFTILLFFLLHRWCSDKKNASVMDQGPAGNRTVNREDSDE

SEQ ID NO: 670 - protein, 304 aa, Synthetic construct [sequence omitted]

SEQ ID NO: 671 - protein, 304 aa, Synthetic construct [sequence omitted]

SEQ ID NO: 672 - protein, 382 aa, Synthetic construct [sequence omitted]

SEQ ID NO: 673 - protein, 17 aa, Synthetic construct
MWLQNLLLLGAVVCSIS

SEQ ID NO: 674 - protein, 20 aa, Synthetic construct
METPAQLLFLLLLWLPDTTG

SEQ ID NO: 675 - protein, 33 aa, Synthetic construct
MAALQKSVSSFLMGTLATSCLLLLALLVQGGAA

SEQ ID NO: 676 - protein, 19 aa, Synthetic construct
ATNFSLLKQAGDVEENPGP

SEQ ID NO: 677 - protein, 20 aa, Synthetic construct
QCTNYALLKLAGDVESNPGP

SEQ ID NO: 678 - protein, 18 aa, Synthetic construct
EGRGSLLTCGDVEENPGP

SEQ ID NO: 679 - protein, 22 aa, Synthetic construct
VKQTLNFDLLKLAGDVESNPGP

SEQ ID NO: 680 - protein, 894 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGATNFSLLKQAGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 681 - protein, 894 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGATNFSLLKQAGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 682 - protein, 895 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGATNFSLLKQAGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGATNFSLLKQAGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 683 - protein, 895 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGATNFSLLKQAGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGATNFSLLKQAGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 684 - protein, 893 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 685 - protein, 893 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 686 - protein, 894 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGATNFSLLKQAGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 687 - protein, 894 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGATNFSLLKQAGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 688 - DNA, 2690 bp, Synthetic construct
  source 1..2690: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagccacgaacttctctctgttaaagcaagcaggagacgtggaagaaaaccccggtcctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 689 - DNA, 2690 bp, Synthetic construct
  source 1..2690: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagccacgaacttctctctgttaaagcaagcaggagacgtggaagaaaaccccggtcctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 690 - DNA, 2693 bp, Synthetic construct
  source 1..2693: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagccacgaacttctctctgttaaagcaagcaggagacgtggaagaaaaccccggtcctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagccacgaacttctctctgttaaagcaagcaggagacgtggaagaaaaccccggtcctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 691 - DNA, 2693 bp, Synthetic construct
  source 1..2693: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagccacgaacttctctctgttaaagcaagcaggagacgtggaagaaaaccccggtcctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagccacgaacttctctctgttaaagcaagcaggagacgtggaagaaaaccccggtcctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 692 - DNA, 2687 bp, Synthetic construct
  source 1..2687: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 693 - DNA, 2687 bp, Synthetic construct
  source 1..2687: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagagggcagaggaagtcttctaacatgcggtgacgtggaggagaatcccggccctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 694 - DNA, 2690 bp, Synthetic construct
  source 1..2690: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagccacgaacttctctctgttaaagcaagcaggagacgtggaagaaaaccccggtcctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 695 - DNA, 2690 bp, Synthetic construct
  source 1..2690: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagccacgaacttctctctgttaaagcaagcaggagacgtggaagaaaaccccggtcctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagagggcagaggaagtcttctaacatgcggtgacgtggaggagaatcccggccctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataatgagc

SEQ ID NO: 696 - DNA, 66 bp, Synthetic construct
  source 1..66: mol_type = other DNA
gtgaagcagaccctgaacttcgacctgctgaagctggccggcgacgtggagagcaaccccggcccc

SEQ ID NO: 697 - DNA, 63 bp, Synthetic construct
  source 1..63: mol_type = other DNA
atggccttaccagtgaccgccttgctcctgccgctggccttgctgctccacgccgccaggccg

SEQ ID NO: 698 - protein, 22 aa, Synthetic construct
MLLLVTSLLLCELPHPAFLLIP

SEQ ID NO: 699 - DNA, 66 bp, Synthetic construct
  source 1..66: mol_type = other DNA
atgcttctcctggtgacaagccttctgctctgtgagttaccacacccagcattcctcctgatccca

SEQ ID NO: 700 - DNA, 66 bp, Synthetic construct
  source 1..66: mol_type = other DNA
atgcttctgttggtgacttcactcctgctgtgcgaacttcctcatcccgccttccttttgattcca

SEQ ID NO: 701 - protein, 75 aa, Synthetic construct; identical to SEQ ID NO: 266

SEQ ID NO: 702 - DNA, 225 bp, Synthetic construct
  source 1..225: mol_type = other DNA
tcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgca

SEQ ID NO: 703 - DNA, 225 bp, Synthetic construct
  source 1..225: mol_type = other DNA
tcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgca

SEQ ID NO: 704 - protein, 99 aa, Synthetic construct
PYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 705 - DNA, 297 bp, Synthetic construct
  source 1..297: mol_type = other DNA
ccctatgagtggtcaaactcgagtgacccactgcttgtttctgtcacaggaaacccttcaaatagttggccttcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgca

SEQ ID NO: 706 - DNA, 297 bp, Synthetic construct
  source 1..297: mol_type = other DNA
ccctatgagtggtcaaactcgagtgacccactgcttgtttctgtcacaggaaacccttcaaatagttggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgca

SEQ ID NO: 707 - protein, 506 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 708 - protein, 500 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGASHHHHHHPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 709 - protein, 506 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 710 - protein, 530 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 711 - protein, 894 aa, Synthetic construct; identical to SEQ ID NO: 680

SEQ ID NO: 712 - protein, 894 aa, Synthetic construct; identical to SEQ ID NO: 681

SEQ ID NO: 713 - protein, 895 aa, Synthetic construct; identical to SEQ ID NO: 682

SEQ ID NO: 714 - protein, 895 aa, Synthetic construct; identical to SEQ ID NO: 683

SEQ ID NO: 715 - protein, 893 aa, Synthetic construct; identical to SEQ ID NO: 684

SEQ ID NO: 716 - protein, 893 aa, Synthetic construct; identical to SEQ ID NO: 685

SEQ ID NO: 717 - protein, 894 aa, Synthetic construct; identical to SEQ ID NO: 686

SEQ ID NO: 718 - protein, 894 aa, Synthetic construct; identical to SEQ ID NO: 687

SEQ ID NO: 719 - protein, 924 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGATNFSLLKQAGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 720 - protein, 924 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGATNFSLLKQAGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYAVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 721 - protein, 924 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYAVEGGGATNFSLLKQAGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 722 - protein, 924 aa, Synthetic construct
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGATNFSLLKQAGDVEENPGPRMALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYAVEGGGEGRGSLLTCGDVEENPGPRMALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 723 - DNA, 1521 bp, Synthetic construct
  source 1..1521: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 724 - DNA, 1503 bp, Synthetic construct
  source 1..1503: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggagctagccaccatcaccatcaccatcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 725 - DNA, 1521 bp, Synthetic construct
  source 1..1521: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggcggcagcggcgggggagggtccggagggggtggttctggtggaggaggatcgggaggcggtggcagctcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgcataa

SEQ ID NO: 726 - DNA, 1593 bp, Synthetic construct
  source 1..1593: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggcggcagcggcgggggagggtccggagggggtggttctggtggaggaggatcgggaggcggtggcagcccctatgagtggtcaaactcgagtgacccactgcttgtttctgtcacaggaaacccttcaaatagttggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgcataa

SEQ ID NO: 727 - DNA, 2690 bp, Synthetic construct; identical to SEQ ID NO: 688
  source 1..2690: mol_type = other DNA

SEQ ID NO: 728 - DNA, 2690 bp, Synthetic construct; identical to SEQ ID NO: 689
  source 1..2690: mol_type = other DNA

SEQ ID NO: 729 - DNA, 2693 bp, Synthetic construct; identical to SEQ ID NO: 691
  source 1..2693: mol_type = other DNA

SEQ ID NO: 730 - skipped

SEQ ID NO: 731 - DNA, 2687 bp, Synthetic construct; identical to SEQ ID NO: 692
  source 1..2687: mol_type = other DNA

SEQ ID NO: 732 - DNA, 2687 bp, Synthetic construct; identical to SEQ ID NO: 693
  source 1..2687: mol_type = other DNA

SEQ ID NO: 733 - DNA, 2690 bp, Synthetic construct; identical to SEQ ID NO: 694
  source 1..2690: mol_type = other DNA

SEQ ID NO: 734 - DNA, 2690 bp, Synthetic construct; identical to SEQ ID NO: 695
  source 1..2690: mol_type = other DNA

SEQ ID NO: 735 - DNA, 2775 bp, Synthetic construct
  source 1..2775: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagccacgaacttctctctgttaaagcaagcaggagacgtggaagaaaaccccggtcctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggcggcagcggcgggggagggtccggagggggtggttctggtggaggaggatcgggaggcggtggcagcccctatgagtggtcaaactcgagtgacccactgcttgtttctgtcacaggaaacccttcaaatagttggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgcataa

SEQ ID NO: 736 - DNA, 2775 bp, Synthetic construct
  source 1..2775: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagccacgaacttctctctgttaaagcaagcaggagacgtggaagaaaaccccggtcctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagagggcagaggaagtcttctaacatgcggtgacgtggaggagaatcccggccctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggcggcagcggcgggggagggtccggagggggtggttctggtggaggaggatcgggaggcggtggcagcccctatgagtggtcaaactcgagtgacccactgcttgtttctgtcacaggaaacccttcaaatagttggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgcataa

SEQ ID NO: 737 - DNA, 2775 bp, Synthetic construct
  source 1..2775: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggcggcagcggcgggggagggtccggagggggtggttctggtggaggaggatcgggaggcggtggcagcccctatgagtggtcaaactcgagtgacccactgcttgtttctgtcacaggaaacccttcaaatagttggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgcagtcgagggcggcggagccacgaacttctctctgttaaagcaagcaggagacgtggaagaaaaccccggtcctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 738 - DNA, 2775 bp, Synthetic construct
  source 1..2775: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagccacgaacttctctctgttaaagcaagcaggagacgtggaagaaaaccccggtcctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggcggcagcggcgggggagggtccggagggggtggttctggtggaggaggatcgggaggcggtggcagcccctatgagtggtcaaactcgagtgacccactgcttgtttctgtcacaggaaacccttcaaatagttggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgcagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 739 - protein, 16 aa, Synthetic construct
SPTEPSSKTGNPRHLH

SEQ ID NO: 740 - protein, 40 aa, Synthetic construct
PYEWSNSSDPLLVSVTGNPSNSWPSPTEPSSKTGNPRHLH

SEQ ID NO: 741 - protein, 21 aa, Synthetic construct
VLIGTSVVKIPFTILLFFLLH

SEQ ID NO: 742 - protein, 38 aa, Synthetic construct
RWCSNKKNAAVMDQEPAGNRTVNSEDSDEQDHQEVSYA

SEQ ID NO: 743 - protein, 99 aa, Synthetic construct; identical to SEQ ID NO: 704

SEQ ID NO: 744 - protein, 5 aa, Synthetic construct
VEGGG

SEQ ID NO: 745 - protein, 10 aa, Synthetic construct
EQKLISEEDL

SEQ ID NO: 746 - protein, 40 aa, Synthetic construct
MGGLEPCSRFLLLPLLLAVSGLRPVQVQAQSDCSCSTVSP

SEQ ID NO: 747 - protein, 21 aa, Synthetic construct
GVLAGIVMGDLVLTVLIALAV

SEQ ID NO: 748 - protein, 52 aa, Synthetic construct
YFLGRLVPRGRGAAEAATRKQRITETESPYQELQGQRSDVYSDLNTQRPYYK

SEQ ID NO: 749 - protein, 6 aa, Synthetic construct
MGDLVL

SEQ ID NO: 750 - protein, 18 aa, Synthetic construct
ESPYQELQGQRSDVYSDL

SEQ ID NO: 751 - protein, 6 aa, Synthetic construct
SPYQEL

SEQ ID NO: 752 - protein, 5 aa, Synthetic construct
VVKIP

SEQ ID NO: 753 - protein, 20 aa, Synthetic construct
METPAQLLFLLLLWLPDTTG

SEQ ID NO: 754 - protein, 6 aa, Synthetic construct
HHHHHH

SEQ ID NO: 755 - protein, 8 aa, Synthetic construct
DYKDDDDK

SEQ ID NO: 756 - protein, 8 aa, Synthetic construct
WSHPQFEK

SEQ ID NO: 757 - protein, 38 aa, Synthetic construct
MDEKTTGWRGGHVVEGLAGELEQLRARLEHHPQGQREP

SEQ ID NO: 758 - protein, 9 aa, Synthetic construct
YPYDVPDYA

SEQ ID NO: 759 - protein, 230 aa, Synthetic construct
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 760 - protein, 18 aa, Synthetic construct
MEPLRLLILLFVTELSGA

SEQ ID NO: 761 - protein, 156 aa, Synthetic construct [sequence omitted]

SEQ ID NO: 762 - protein, 84 aa, Synthetic construct
GQSLQVSCPYDSMKHWGRRKAWCRQLGEKGPCQRVVSTHNLWLLSFLRRWNGSTAITDDTLGGTLTITLRNLQPHDAGLYQCQS

SEQ ID NO: 763 - protein, 45 aa, Synthetic construct
ADPLDHRDAGDLWFPGESESFEDAHVEHSISRSLLEGEIPFPPTS

SEQ ID NO: 764 - protein, 21 aa, Synthetic construct
ILLLLACIFLIKILAASALWA

SEQ ID NO: 765 - protein, 35 aa, Synthetic construct
AAWHGQKPGTHPPSELDCGHDPGYQLQTLPGLRDT

SEQ ID NO: 766 - protein, 5 aa, Synthetic construct
LIKIL

SEQ ID NO: 767 - protein, 71 aa, Synthetic construct
KGFSGTPGSNENSTQNVYKIPPTTTKALCPLYTSPRTVTQAPPKSTADVSTPDSEINLTNVTDIIRVPVFN

SEQ ID NO: 768 - protein, 21 aa, Synthetic construct
TPDSEINLTNVTDIIRVPVFN

SEQ ID NO: 769 - protein, 21 aa, Synthetic construct
IVILLAGGFLSKSLVFSVLFA

SEQ ID NO: 770 - protein, 8 aa, Synthetic construct
VTLRSFVP

SEQ ID NO: 771 - protein, 5 aa, Synthetic construct
LSKSL

SEQ ID NO: 772 - protein, 63 aa, Synthetic construct
VAAILGLGLVLGLLGPLAILLALYLLRRDQRLPPDAHKPPGGGSFRTPIQEEQADAHSTLAKI

SEQ ID NO: 773 - protein, 21 aa, Synthetic construct
VAAILGLGLVLGLLGPLAILL

SEQ ID NO: 774 - protein, 42 aa, Synthetic construct
ALYLLRRDQRLPPDAHKPPGGGSFRTPIQEEQADAHSTLAKI

SEQ ID NO: 775 - protein, 142 aa, Synthetic construct
IYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 776 - protein, 26 aa, Synthetic construct
IYLIIGICGGGSLLMVFVALLVFYIT

SEQ ID NO: 777 - protein, 116 aa, Synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 778 - protein, 69 aa, Synthetic construct
ILVIFSGMFLVFTLAGALFLHQRRKYRSNKGESPVEPAEPCHYSCPREEEGSTIPIQEDYRKPEPACSP

SEQ ID NO: 779 - protein, 21 aa, Synthetic construct
ILVIFSGMFLVFTLAGALFLH

SEQ ID NO: 780 - protein, 48 aa, Synthetic construct
QRRKYRSNKGESPVEPAEPCHYSCPREEEGSTIPIQEDYRKPEPACSP

SEQ ID NO: 781 - protein, 107 aa, Synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 782 - protein, 39 aa, Synthetic construct
IEVMYPPPYLDNEKSNGTIIHVKGKHLCPSPLFPGPSKP

SEQ ID NO: 783 - protein, 27 aa, Synthetic construct
FWVLVVVGGVLACYSLLVTVAFIIFWV

SEQ ID NO: 784 - protein, 41 aa, Synthetic construct
RSKRSRLLHSDYMNMTPRRPGPTRKHYQPYAPPRDFAAYRS

SEQ ID NO: 785 - protein, 93 aa, Synthetic construct
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 786 - protein, 21 aa, Synthetic construct
LLAGLVAADAVASLLIVGAVF

SEQ ID NO: 787 - protein, 24 aa, Synthetic construct
LCARPRRSPAQEDGKVYINMPGRG

SEQ ID NO: 788 - protein, 5 aa, Synthetic construct
AADAV

SEQ ID NO: 789 - protein, 216 aa, Synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 790 - protein, 116 aa, Synthetic construct
PCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQ

SEQ ID NO: 791 - protein, 25 aa, Synthetic construct
IWSAVFLNSLFNQEVQIPLTESYCG

SEQ ID NO: 792 - protein, 21 aa, Synthetic construct
PFFFCCFIAVAMGIRFIIMVT

SEQ ID NO: 793 - protein, 51 aa, Synthetic construct
MGWIRGRRSRHSWEMSEFHNYNLDLKKSDFSTRWQKQRCPVVKSKCRENAS

SEQ ID NO: 794 - protein, 5 aa, Synthetic construct
GIRFI

SEQ ID NO: 795 - DNA, 8944 bp, Synthetic construct
  source 1..8944: mol_type = other DNA
ttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggactctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggccctctgatgagacgaacgtgagaccgaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttataggtctcagctgcgtctcaagcatacacgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtttctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccatctccgggagctgcatgtgtcagaggttttcaccgtcatcaccgaaacgcgcgagatgaaagggcctcgtgatacgcctatttttataggttaatgtcatgataataatggtttcttagacgtcaggtggcacttttcggggaaatgtgccgagcggccgcacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatcgcggccgccaattttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcat

SEQ ID NO: 796 - DNA, 8001 bp, Synthetic construct
  source 1..8001: mol_type = other DNA
agagttaggcagggatattcaccattatcgtttcagacccacctcccaaccccgaggggaccctatcatatgctgggtaccctgtctagagcgaacggagacgactagtgatatccgtctctcggcaggagaagctttaggcgtgtacggtgggcgcctataaaagcagagctcgtttagtgaaccgtcagatcgcctggagcaattccacaacacttttgtcttataccaactttccgtaccacttcctaccctcgtaaactggaggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatcgtaccccgaatttatgctcatggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctggtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagtaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagwrykcttccgatctattggaattagataaatgggcaagtttgtggaattggtttaacataacaaattggctgtggtatataaaattattcataatgatagtaggaggcttggtaggtttaagaatagtttttgctgtactttctatagtgaat

SEQ ID NO: 797 - DNA, 9286 bp, Synthetic construct
  source 1..9286: mol_type = other DNA
caaaaattcaaaattttcgggtttattacagggacagcagagatccactttggcgccggctcgagggggcccgggtgcaaagatggataaagttttaaacagagaggaatctttgcagctaatggaccttctaggtcttgaaaggagtgggaattggctccggtgcccgtcagtgggcagagcgcacatggcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgttggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccgagggcaggggaagtctgctaacatgcggggacgtggaggaaaatcccggccccgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaggacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgaaaccctgtaccccgctgacggcggcctggaaggcagagccgacatggccctgaagctcgtgggcgggggccacctgatctgcaacttgaaaaccacatacagatccaagaaacccgctaagaacctcaagatgcccggcgtctactatgtggacagaaggctggaaagaatcaaggaggccgacaaagaaacctacgtcgagcagcacgaggtggctgtggccagatactgcgacctccctagcaaactggggcacagataaggatccacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcaacgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtgttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgatgacaagatctgctttttgcttgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggcgatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggatgagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgttgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagagcaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttgtaaagatatctgagcacacaaaatacagaattccatgctaaaatattgttcctgttccattaactgcgctaaaaataaacgtctctgtttgagagctatgctggaaacagcatagcaagttcaaataaggctagtccgttatcaacttgaaaaagtggcaccgagtcggtgctcggaaaaaaaagaaaaaaagaaaaaaagaaaaaaatccatgaccaaattggtccatggattcacccacccctttttatttttttcaccgtgggcaggtgaatcgatacacctgcatgggcgattctagaaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaatta

SEQ ID NO: 798 - DNA, 738 bp, Synthetic construct
  source 1..738: mol_type = other DNA
gaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggacagcgcggattactactgcgctaggtccaattattatggctcctcatactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcatctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaa

SEQ ID NO: 799 - protein, 10 aa, Synthetic construct
RASSSVNYMD

SEQ ID NO: 800 - protein, 7 aa, Synthetic construct
ATSNLAS

SEQ ID NO: 801 - protein, 9 aa, Synthetic construct
QQWSFNPPT

SEQ ID NO: 802 - protein, 166 aa, Synthetic construct
QSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYSYNMH

SEQ ID NO: 803 - protein, 17 aa, Synthetic construct
AIYPGNGDTSYNQKFKG

SEQ ID NO: 804 - protein, 13 aa, Synthetic construct
SNYYGSSYWFFDV

SEQ ID NO: 805 - protein, 18 aa, Synthetic construct
GSTSGGGSGGGSGGGGSS

SEQ ID NO: 806 - skipped

SEQ ID NO: 807 - protein, 5 aa, Synthetic construct
PRRAR

SEQ ID NO: 808 - protein, 393 aa, Synthetic construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKPSGGGGSGGGGSGGGGSGGGGSGGGGSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 809 - DNA, 2772 bp, Synthetic construct
  source 1..2772: mol_type = other DNA
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatcccagtctgcgctgactcaaacggcctcagtgtctgggtccctgggccagaaggtcaccatctcctgcactggaagcaggtccaacatcggtgcaaattatgtgggctggtatcaacaggtcccaggaataggccctagaaccgtcatttttggttataattatcgaccttcaggggtccccgatcgattctctagctccaagtcaggcaattcagccaccctgaccatctctgggctccaggctgaggacgaggctgagtattattgttcaacatgggacagcagtctcaacgcttacgtgttcggctcagggacccagctgaccgtcctcggcggtggttcctctagatcttcctcctctggtggcggtggctcgggcggtggtggggaggtgccactggtggaatctgggggagacctggtgaagcctggggggtccttgagactgtcctgtgtggcctctggattcaccctcagtaactatggcatgagctgggtccgtcagtctccagggaaggggctgcagtgggtcgcagctattaggagtggtggaaacacatactacgcagacgctgtgaagggccgattcaccatctccagagacaacgccaagaacacgctgtatctgcagatgaacagcctgagagccgaggacacggccgtgtattactgtgcgtcccaatggggtgattggggccactttgagtactggggccagggcaccctggtcaccgtctcctcagctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcagtcgagggcggcggagccacgaacttctctctgttaaagcaagcaggagacgtggaagaaaaccccggtcctaggatggcactccccgtcaccgcgctcctcttgcccctcgcgctgctccttcatgctgcgaggccggaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggacagcgcggattactactgcgctaggtccaattattatggctcctcatactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcatctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaacctagcggaggcggtggcagcggcggtggagggtcaggagggggtggttctggtggaggaggatcgggaggcggtggaagcccatacgaatggtctaattcctctgaccctctccttgtgtccgtgaccgggaatccttccaactcctggccttcaccaacagagccgtcaagtaaaaccgggaacccgagacatctccacgttctgatcggaacatccgtcgtaaaaatccctttcaccatattgttgtttttcttgttgcaccgatggtgctctaacaagaagaacgcagccgtaatggaccaagagccagccggaaacaggactgtcaattccgaagattccgatgagcaggatcaccaagaagttagctacgca

SEQ ID NO: 810 - DNA, 26 bp, Synthetic construct
  source 1..26: mol_type = other DNA
sggggsggggsggggsggggsggggs

SEQ ID NO: 811 - protein, 11 aa, Synthetic construct
RASQSVSSYLA

SEQ ID NO: 812 - protein, 7 aa, Synthetic construct
DASNRAT

SEQ ID NO: 813 - protein, 9 aa, Synthetic construct
QQRSNWPIT

SEQ ID NO: 814 - protein, 6 aa, Synthetic construct
HDYAMH

SEQ ID NO: 815 - protein, 17 aa, Synthetic construct
TISWNSGTIGYADSVKG

SEQ ID NO: 816 - protein, 13 aa, Synthetic construct
DIQYGNYYYGMDV

SEQ ID NO: 817 - protein, 7 aa, Synthetic construct
GMSNLVS

SEQ ID NO: 818 - protein, 245 aa, Synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 819 - DNA, 738 bp, Synthetic construct
  source 1..738: mol_type = other DNA
gaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggttcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaa

SEQ ID NO: 820 - DNA, 15 bp, Synthetic construct
  source 1..15: mol_type = other DNA
ggtggcggaggttct

SEQ ID NO: 821 - DNA, 15 bp, Synthetic construct
  source 1..15: mol_type = other DNA
ggaggtgggggttcc

SEQ ID NO: 822 - DNA, 15 bp, Synthetic construct
  source 1..15: mol_type = other DNA
ggaggcggcggcagc

SEQ ID NO: 823 - DNA, 15 bp, Synthetic construct
  source 1..15: mol_type = other DNA
ggcgggggagggtcc

SEQ ID NO: 824 - DNA, 15 bp, Synthetic construct
  source 1..15: mol_type = other DNA
ggagggggtggttct

SEQ ID NO: 825 - DNA, 15 bp, Synthetic construct
  source 1..15: mol_type = other DNA
ggtggaggaggatcg

SEQ ID NO: 826 - DNA, 15 bp, Synthetic construct
  source 1..15: mol_type = other DNA
ggaggcggtggcagc

SEQ ID NO: 827 - DNA, 15 bp, Synthetic construct
  source 1..15: mol_type = other DNA
ggcagcggcggcagc

SEQ ID NO: 828 - DNA, 12 bp, Synthetic construct
  source 1..12: mol_type = other DNA
ggcggtggttcc

SEQ ID NO: 829 - DNA, 19 bp, Synthetic construct
  source 1..19: mol_type = other DNA
gggssrssssggggsgggg

SEQ ID NO: 830 - DNA, 57 bp, Synthetic construct
  source 1..57: mol_type = other DNA
ggcggtggttcctctagatcttcctcctctggtggcggtggctcgggcggtggtggg

SEQ ID NO: 831 - protein, 18 aa, Synthetic construct
GSTSGSGKPGSGEGSTKG

SEQ ID NO: 832 - DNA, 54 bp, Synthetic construct
  source 1..54: mol_type = other DNA
ggcagcacatccggttccgggaagcccggctccggagagggcagcaccaagggg

SEQ ID NO: 833 - DNA, 57 bp, Synthetic construct
  source 1..57: mol_type = other DNA
gccacgaacttctctctgttaaagcaagcaggagacgtggaagaaaaccccggtcct

SEQ ID NO: 834 - DNA, 60 bp, Synthetic construct
  source 1..60: mol_type = other DNA
cagtgcaccaactacgccctgctgaagctggccggcgacgtggagagcaaccccggcccc

SEQ ID NO: 835 - DNA, 54 bp, Synthetic construct
  source 1..54: mol_type = other DNA
gagggcagaggaagtcttctaacatgcggtgacgtggaggagaatcccggccct

SEQ ID NO: 836 - protein, 22 aa, Synthetic construct
MLLLVTSLLLCELPHPAFLLIP

SEQ ID NO: 837 - protein, 5 aa, Synthetic construct
GSGGG

SEQ ID NO: 838 - protein, 5 aa, Synthetic construct
SGGGS

SEQ ID NO: 839 - protein, 10 aa, Synthetic construct
SGGGSSGGGS

SEQ ID NO: 840 - protein, 75 aa, Synthetic construct
GGGGSGGGGSGGGGSGGGGSGGGGSGGGGSGGGGSGGGGSGGGGSGGGGSGGGGSGGGGSGGGGSGGGGSGGGGS

SEQ ID NO: 841 - protein, 5 aa, Synthetic construct
GGGGS

SEQ ID NO: 842 - protein, 10 aa, Synthetic construct
GGGGSGGGGS

SEQ ID NO: 843 - protein, 15 aa, Synthetic construct
GGGGSGGGGSGGGGS

SEQ ID NO: 844 - protein, 25 aa, Synthetic construct
GGGGSGGGGSGGGGSGGGGSGGGGS

SEQ ID NO: 845 - protein, 4 aa, Synthetic construct
GGGS